(19) United States
(12) Patent Application Publication
(10) Pub. No.: US 2024/0382620 A1
(43) Pub. Date: Nov. 21, 2024

(54) GENOME EDITING COMPOSITIONS AND METHODS FOR TREATMENT OF USHER SYNDROME TYPE 3

(71) Applicant: Prime Medicine, Inc., Cambridge, MA (US)

(72) Inventor: Wei Hsi Yeh, Cambridge, MA (US)

(21) Appl. No.: 18/702,703

(22) PCT Filed: Oct. 20, 2022
(86) PCT No.: PCT/US22/78473

§ 371 Date: Apr. 18, 2024

Related U.S. Application Data
(60) Provisional application No. 63/270,368, filed on Oct. 21, 2021.

(51) Int. Cl. A61K 48/00; C12N 9/12; C12N 9/22; C12N 15/11; C12N 15/90

(52) U.S. Cl. CPC A61K 48/005; C12N 9/1276; C12N 9/22; C12N 15/11; C12N 15/907; C12N 2310/20; C12N 2310/313; C12N 2310/321; C12N 2310/351; C12N 2310/531

(57) ABSTRACT

Provided herein are compositions and methods of using prime editing systems comprising prime editors and prime editing guide RNAs for treatment of genetic disorders.

### CROSS-REFERENCE

This application claims the benefit of U.S. Provisional Application No. 63/270,368, filed Oct. 21, 2021, which is incorporated herein by reference in its entirety.

### BACKGROUND

Usher syndrome is an autosomal recessive disorder involving dual impairment of the visual and audiovestibular systems and is the most common cause of deaf-blindness. Patients with Usher syndrome often have congenital sensorineural hearing loss with or without vestibular dysfunction, and visual loss in the form of retinitis pigmentosa (RP).

Diseases, such as Usher Syndrome type 3, may be caused in humans by disruption to the CLRN1 gene (OMIM #606397), and manifest as sensorineural hearing loss and visual impairment from retinitis pigmentosa. CLRN1 is mainly expressed in inner and outer hair cells of the inner ear and in photoreceptor cells and Müller cells of the retina and related tissues and encodes the clarin-1 protein. Clarin-1 is expressed in multiple isoforms due to alternative splicing. Exemplary isoforms include isoform a, NCBI ref. NP_777367, SEQ ID NO: 674; isoform d, NCBI ref. NP_001182723, SEQ ID NO: 676; and isoform e, NCBI ref NP_001243748, SEQ ID NO: 678. CLRN1 is located in the human genome at 3q25.1 (chr3:150,926,163-150,972,999 (GRCh38/hg38)). CLRN1 mRNA isoform transcript range in size from about 2 kb to 2.4 kb (isoform a: NM_174878, SEQ ID NO: 675; isoform d: NM_001195794, SEQ ID NO: 677; isoform e: NM_001256819, SEQ ID NO: 679). A frequent disease-causing mutation of CLRN1 is N48K, in which a T-to-G transversion at position 144 of the coding sequence in exon 0 (Chr3:150,972,565, GRCh38) causes a missense mutation in codon 48 from asparagine (AAT) to lysine (AAG).

### SUMMARY

Usher Syndrome type 3 can be treated by gene editing because the N48K mutation in the CLRN1 gene is amenable to prime editing, methods and compositions for which are described herein. The N48K mutation in CLRN1 may be corrected, for example, by a G->T edit at position 144 of the coding sequence, thus restoring the missense mutation to wild-type.

Provided herein, in some embodiments, are methods and compositions for prime editing of alterations in a target sequence in a target gene, for example, the CLRN1 gene. The target CLRN1 gene may comprise double stranded DNA. As exemplified in FIG. 1, in an embodiment, the target gene is edited by prime editing.

Without wishing to be bound by any particular theory, the prime editing process may search specific targets and edit endogenous sequences in a target gene, e.g., the CLRN1 gene. As exemplified in FIG. 1, the spacer sequence of a PEgRNA recognizes and anneals with a search target sequence in a target strand of the target gene. A prime editing complex may generate a nick in the target gene on the edit strand which is the complementary strand of the target strand. The prime editing complex may then use a free 3′ end formed at the nick site of the edit strand to initiate DNA synthesis, where a primer binding site (PBS) of the PEgRNA complexes with the free 3′ end, and a single stranded DNA is synthesized using an editing template of the PEgRNA as a template. The editing template may comprise one or more nucleotide edits compared to the endogenous target CLRN1 gene sequence. Accordingly, the newly-synthesized single stranded DNA also comprises the nucleotide edit(s) encoded by the editing template. Through removal of an editing target sequence on the edit strand of the target gene and DNA repair, the intended nucleotide edit(s) included in the newly synthesized single stranded DNA are incorporated into the target CLRN1 gene.

In one aspect, provided herein is a prime editing guide RNA (PEgRNA) or a nucleic acid encoding the PEgRNA, wherein the PEgRNA comprises (a) a spacer that is complementary to a search target sequence on a first strand of a CLRN1 gene wherein the spacer comprises at its 3′ end SEQ ID NO: 1; (b) a gRNA core capable of binding to a Cas9 protein; and (c) an extension arm comprising: (i) an editing template that comprises a region of complementarity to an editing target sequence on a second strand of the CLRN1 gene, and (ii) a primer binding site (PBS) that comprises at its 5′ end a sequence that is a reverse complement of nucleotides 10-14 of SEQ ID NO: 1, wherein the first strand and second strand are complementary to each other, wherein the editing target sequence on the second strand comprises or is complementary to a portion of the CLRN1 gene comprising a c. 144 T->G substitution, and wherein the editing template encodes or comprises a wild-type amino acid sequence of a Clarin 1 protein at the c. 144 T->G substitution. In some embodiments, the spacer comprises at its 3′ end any one of SEQ ID NOs: 2-6. In some embodiments, the spacer comprises at its 3′ end SEQ ID NO: 4. In some embodiments, the editing template comprises SEQ ID NO: 22 at its 3′ end and encodes an AGG to ATG PAM silencing edit. In some embodiments, the editing template comprises at its 3′ end SEQ ID NO: 27, 34, 38, 43, 47, 53, 58, 62, 66, 70, 74, 78, 82, 86, 90, 94, 98,102,106, 110, 114, 118, 122, 126, 130, 134, 138, or 142. In some embodiments, the editing template comprises SEQ ID NO: 23 at its 3′ end. In some embodiments, the editing template comprises at its 3′ end SEQ ID NO: 28, 31, 35, 39, 44, 48, 54, 59, 63, 67, 71, 75, 79, 83, 87, 91, 95, 99,103,107, 111, 115, 119, 123, 127, 131, 135, 139, or 143. In some embodiments, the editing template comprises SEQ ID NO: 24 at its 3′ end and encodes an AGG-to-ACG PAM silencing edit. In some embodiments, the editing template comprises at its 3′ end SEQ ID NO: 29, 36, 40, 45, 49, 55, 60, 64, 68, 72, 76, 80, 84, 88, 92, 96,100,104, 108, 112, 116, 120, 124, 128, 132, 136, 140, or 144. In some embodiments, the editing template comprises SEQ ID NO: 25 at its 3′ end and encodes an AGG-to-AAG PAM silencing edit. In some embodiments, the editing template comprises at its 3′ end SEQ ID NO: 30, 37, 41, 46, 50, 56, 61, 65, 69, 73, 77, 81, 85, 89, 93, 97,101,105, 109, 113, 117, 121, 125, 129, 133, 137, 141, or 145. In some embodiments, the editing template comprises SEQ ID NO: 26 at its 3′ end and encodes a AGG-to-AGC PAM silencing edit. In some embodiments, the editing template comprises at its 3′ end SEQ ID NO: 32, 33, 42, 51, 52, or 57. In some embodiments, the editing template comprises at its 3′ end any one of sequences set forth in SEQ ID NOs: 22 to 145. In some embodiments, wherein the editing template has a length of 40 nucleotides or less. In some embodiments, the editing template has a length of 26 nucleotide or less. In some embodiments, the editing template is 12 to 26 nucleotides in length. In some embodiments, the editing template has a length of 18 nucleotides or less. In some embodiments, the editing template is 12 to 18 nucleotides in length. In some embodiments, the PBS comprises at its 5′end a sequence corresponding to sequence number 7. In some embodiments, the PBS comprises sequence number 8, 9, 10, 11, 12 (SEQ ID NO: 12), 13 (SEQ ID NO: 13), 14 (SEQ ID NO: 14), 15 (SEQ ID NO: 15), 16 (SEQ ID NO: 16), 17 (SEQ ID NO: 17), 18 (SEQ ID NO: 18), 19 (SEQ ID NO: 19), 20 (SEQ ID NO: 20), or 21 (SEQ ID NO: 21). In some embodiments, the PBS has a length of 16 nucleotides or less. In some embodiments, the PBS is 8 to 16 nucleotides in length. In some embodiments, the PBS has a length of 15 nucleotides or less. In some embodiments, the PBS is 9 to 15 nucleotides in length. In some embodiments, the spacer of the PEgRNA is from 17 to 22 nucleotides in length. In some embodiments, the spacer of the PEgRNA is 20 nucleotides in length. In some embodiments, the gRNA core comprises any one of SEQ ID NOs: 665-669. In some embodiments, the PEgRNA comprises from 5′ to 3′, the spacer, the gRNA core, the editing template, and the PBS. In some embodiments, the spacer, the gRNA core, the editing template, and the PBS form a contiguous sequence in a single molecule. In some embodiments, the PEgRNA further comprises a linker sequence at the 3′ end. In some embodiments, the linker sequence comprises a sequence set forth at Sequence Number 671. In some embodiments, the PEgRNA further comprises a hairpin motif at the 3′ end. In some embodiments, the hairpin motif comprises a sequence set forth at SEQ ID NO: 672. In some embodiments, the PEgRNA further comprises 3′ mN*mN*mN*N and 5′mN*mN*mN* modifications, where m indicates that the nucleotide contains a 2′-O-Me modification and a * indicates the presence of a phosphorothioate bond. In some embodiments, the PEgRNA further comprises 3′ mT*mT*mT*T and 5′mN*mN*mN* modifications, where m indicates that the nucleotide contains a 2′-O-Me modification, a * indicates the presence of a phosphorothioate bond, and a T indicates the presence of an additional uridine nucleotide. In some embodiments, the editing template encodes a PAM silencing edit. In some embodiments, a PEgRNA sequence is selected from any one of SEQ ID NOs: 195-508.

In one aspect, provided herein is a prime editing guide RNA (PEgRNA), or a nucleic acid encoding the PEgRNA, wherein the PEgRNA comprises: a) a spacer comprising at its 3′ end SEQ ID NO: 1; b) a gRNA core capable of binding to a Cas9 protein; and c) an extension arm comprising: (i) an editing template comprising at its 3′ end any one of SEQ ID NOs: 22-26, and (ii) a primer binding site (PBS) comprising at its 5′ end a sequence that is a reverse complement of nucleotides 10-14 of SEQ ID NO: 1. In some embodiments, the spacer comprises at its 3′ end any one of SEQ ID NOs: 2-6. In some embodiments, the spacer comprises at its 3′ end SEQ ID NO: 4. In some embodiments, the editing template comprises SEQ ID NO: 22 at its 3′ end and encodes an AGG to ATG PAM silencing edit. In some embodiments, the editing template comprises at its 3′ end SEQ ID NO: 27, 34, 38, 43, 47, 53, 58, 62, 66, 70, 74, 78, 82, 86, 90, 94, 98,102,106, 110, 114, 118, 122, 126, 130, 134, 138, or 142. In some embodiments, the editing template comprises SEQ ID NO: 23 at its 3′ end. In some embodiments, the editing template comprises at its 3′ end SEQ ID NO: 28, 31, 35, 39, 44, 48, 54, 59, 63, 67, 71, 75, 79, 83, 87, 91, 95, 99,103,107, 111, 115, 119, 123, 127, 131, 135, 139, or 143. In some embodiments, the editing template comprises SEQ ID NO: 24 at its 3′ end and encodes an AGG-to-ACG PAM silencing edit. In some embodiments, the editing template comprises at its 3′ end SEQ ID NO: 29, 36, 40, 45, 49, 55, 60, 64, 68, 72, 76, 80, 84, 88, 92, 96,100,104, 108, 112, 116, 120, 124, 128, 132, 136, 140, or 144. In some embodiments, the editing template comprises SEQ ID NO: 25 at its 3′ end and encodes an AGG-to-AAG PAM silencing edit. In some embodiments, the editing template comprises at its 3′ end SEQ ID NO: 30, 37, 41, 46, 50, 56, 61, 65, 69, 73, 77, 81, 85, 89, 93, 97,101,105, 109, 113, 117, 121, 125, 129, 133, 137, 141, or 145. In some embodiments, the editing template comprises SEQ ID NO: 26 at its 3′ end and encodes a AGG-to-AGC PAM silencing edit. In some embodiments, the editing template comprises at its 3′ end SEQ ID NO: 32, 33, 42, 51, 52, or 57. In some embodiments, the editing template comprises at its 3′ end any one of sequences set forth in SEQ ID NOs: 22 to 145. In some embodiments, the editing template has a length of 40 nucleotides or less. In some embodiments, the editing template has a length of 26 nucleotide or less. In some embodiments, the editing template is 12 to 26 nucleotides in length. In some embodiments, the editing template has a length of 18 nucleotides or less. In some embodiments, the editing template is 12 to 18 nucleotides in length. In some embodiments, the PBS comprises at its 5′end a sequence corresponding to sequence number 7. In some embodiments, the PBS comprises sequence number 8, 9, 10, 11, 12 (SEQ ID NO: 12), 13 (SEQ ID NO: 13), 14 (SEQ ID NO: 14), 15 (SEQ ID NO: 15), 16 (SEQ ID NO: 16), 17 (SEQ ID NO: 17), 18 (SEQ ID NO: 18), 19 (SEQ ID NO: 19), 20 (SEQ ID NO: 20), or 21 (SEQ ID NO: 21). In some embodiments, the PBS has a length of 16 nucleotides or less. In some embodiments, the PBS is 8 to 16 nucleotides in length. In some embodiments, the PBS has a length of 15 nucleotides or less. In some embodiments, the PBS is 9 to 15 nucleotides in length. In some embodiments, the spacer of the PEgRNA is from 17 to 22 nucleotides in length. In some embodiments, the spacer of the PEgRNA is 20 nucleotides in length. In some embodiments, the gRNA core comprises any one of SEQ ID NOs: 665-669. In some embodiments, the PEgRNA comprises from 5′ to 3′, the spacer, the gRNA core, the editing template, and the PBS. In some embodiments, the spacer, the gRNA core, the editing template, and the PBS form a contiguous sequence in a single molecule. In some embodiments, the PEgRNA further comprises a linker sequence at the 3′ end. In some embodiments, the linker sequence comprises a sequence set forth at Sequence Number 671. In some embodiments, the PEgRNA further comprises a hairpin motif at the 3′ end. In some embodiments, the hairpin motif comprises a sequence set forth at SEQ ID NO: 672. In some embodiments, the PEgRNA further comprises 3′ mN*mN*mN*N and 5′mN*mN*mN* modifications, where m indicates that the nucleotide contains a 2′-O-Me modification and a * indicates the presence of a phosphorothioate bond. In some embodiments, the PEgRNA further comprises 3′ mT*mT*mT*T and 5′mN*mN*mN* modifications, where m indicates that the nucleotide contains a 2′-O-Me modification, a * indicates the presence of a phosphorothioate bond, and a T indicates the presence of an additional uridine nucleotide. In some embodiments, the editing template encodes a PAM silencing edit. In some embodiments, a PEgRNA sequence is selected from any one of SEQ ID NOs: 195-508.

In one aspect, provided herein is a prime editing guide RNA (PEgRNA) comprising: a) a spacer comprising at its 3′ end any one of a PEgRNA spacer sequence as set forth in Table 1; b) a gRNA core capable of binding to a Cas9 protein; and c) an extension arm comprising: i) an editing template comprising at its 3′ end any one of a RTT sequence as set forth in Table 1; and ii) a primer binding site (PBS) comprising at its 5′ end any one of a PBS sequence as set forth in Table 1. In some embodiments, the spacer of the PEgRNA is from 17 to 22 nucleotides in length. In some embodiments, the spacer of the PEgRNA is 20 nucleotides in length. In some embodiments, the gRNA core comprises any one of SEQ ID NOs: 665-669. In some embodiments, the PEgRNA comprises from 5′ to 3′, the spacer, the gRNA core, the editing template, and the PBS. In some embodiments, the spacer, the gRNA core, the editing template, and the PBS form a contiguous sequence in a single molecule. In some embodiments, the PEgRNA further comprises a linker sequence at the 3′ end. In some embodiments, the linker sequence comprises a sequence set forth at Sequence Number 671. In some embodiments, the PEgRNA further comprises a hairpin motif at the 3′ end. In some embodiments, the hairpin motif comprises a sequence set forth at SEQ ID NO: 672. In some embodiments, the PEgRNA further comprises 3′ mN*mN*mN*N and 5′mN*mN*mN* modifications, where m indicates that the nucleotide contains a 2′-O-Me modification and a * indicates the presence of a phosphorothioate bond. In some embodiments, the PEgRNA further comprises 3′ mT*mT*mT*T and 5′mN*mN*mN* modifications, where m indicates that the nucleotide contains a 2′-O-Me modification, a * indicates the presence of a phosphorothioate bond, and a T indicates the presence of an additional uridine nucleotide. In some embodiments, the editing template encodes a PAM silencing edit. In some embodiments, a PEgRNA sequence is selected from any one of SEQ ID NOs: 195-508.

In one aspect, provided herein is a prime editing system comprising: (a) the PEgRNA or the nucleic acid encoding the PEgRNA of the disclosure or any of the aspects herein, and (b) a ngRNA, or a nucleic acid encoding the ngRNA, wherein the ngRNA comprises: (i) a spacer comprising at its 3′ end a sequence corresponding to nucleotides 4-20 of any one of SEQ ID NO: 146-194; and (ii) an ngRNA core capable of binding a Cas9 protein. In some embodiments, the spacer of the ngRNA comprises at its 3′ end nucleotides 3-20, 2-20, or 1-20 of any one of SEQ ID NO: 146-194. In some embodiments, the spacer of the ngRNA comprises at its 3′ end any one of SEQ ID NOs: 146-194. In some embodiments, the spacer of the ngRNA comprises at its 3′ end nucleotides 3-20, 2-20, or 1-20 of the ngRNA spacer sequence. In some embodiments, the spacer of the ngRNA comprises at its 3′ end the ngRNA spacer sequence. In some embodiments, the spacer of the ngRNA is from 17 to 22 nucleotides in length. In some embodiments, the spacer of the ngRNA is 20 nucleotides in length. In some embodiments, the gRNA core of the ngRNA comprises any one of SEQ ID NOs: 665-669. In some embodiments, the ngRNA comprises any one of the SEQ ID NOs: 509-588. In some embodiments, the prime editing system further comprises: (c) a prime editor comprising: (i) a Cas9 nickase comprising a nuclease inactivating mutation in the HNH domain, or a nucleic acid encoding the Cas9 nickase, and (ii) a reverse transcriptase, or a nucleic acid encoding the reverse transcriptase. In some embodiments, the prime editor is a fusion protein. In some embodiments, the prime editing system further comprises: (c) an N-terminal extein comprising an N-terminal fragment of a prime editor fusion protein and an N-intein or a polynucleotide encoding the N-terminal extein; and (d) a C-terminal extein comprising a C-terminal fragment of the prime editor fusion protein and a C-intein, or a polynucleotide encoding the C-terminal extein; wherein the N-intein and the C-intein of the N-terminal and C-terminal exteins are capable of self-excision to join the N-terminal fragment and the C-terminal fragment to form the prime editor fusion protein, and wherein the prime editor fusion protein comprises a Cas9 nickase and a reverse transcriptase. In some embodiments, the Cas9 nickase comprises an amino acid sequence comprising at least 80%, 85%, 90%, 95%, 96%, 97%, 98%, 99%, or 100% identity to any one of SEQ ID NOs: 593, 594, 596, 597, 599, 600, 602, 603, 605, 606, 608, 609, 611, 612, 614, 615, 617, 618, or 619. In some embodiments, the reverse transcriptase comprises an amino acid sequence comprising at least 80%, 85%, 90%, 95%, 96%, 97%, 98%, 99%, or 100% identity to any one of SEQ ID NOs: 589, 590, or 591. In some embodiments, the sequence identities are determined by Needleman-Wunsch alignment of two protein sequences with Gap Costs set to Existence: 11 Extension: 1 where percent identity is calculated by dividing the number of identities by a length of the alignment.

In one aspect, provided herein is a prime editing system comprising: (a) the prime editing guide RNA (PEgRNA) of the disclosure or any of the aspects herein, or a nucleic acid encoding the PEgRNA; and optionally (b) a nick guide RNA (ngRNA), or a nucleic acid encoding the ngRNA, wherein the ngRNA comprises a spacer comprising at its 3′ end nucleotides 4-20 of any one of a ngRNA spacer sequence set forth in Table 1 and a gRNA core capable of binding to a Cas9 protein. In some embodiments, the prime editing system comprises the ngRNA. In some embodiments, the spacer of the ngRNA comprises at its 3′ end nucleotides 3-20, 2-20, or 1-20 of the ngRNA spacer sequence. In some embodiments, the spacer of the ngRNA comprises at its 3′ end the ngRNA spacer sequence. In some embodiments, the spacer of the ngRNA is from 17 to 22 nucleotides in length. In some embodiments, the spacer of the ngRNA is 20 nucleotides in length. In some embodiments, the gRNA core of the ngRNA comprises any one of SEQ ID NOs: 665-669. In some embodiments, the ngRNA comprises any one of the SEQ ID NOs: 509-588. In some embodiments, the prime editing system further comprises: (c) a prime editor comprising: (i) a Cas9 nickase comprising a nuclease inactivating mutation in the HNH domain, or a nucleic acid encoding the Cas9 nickase, and (ii) a reverse transcriptase, or a nucleic acid encoding the reverse transcriptase. In some embodiments, the prime editor is a fusion protein. In some embodiments, the prime editing system further comprises: (c) an N-terminal extein comprising an N-terminal fragment of a prime editor fusion protein and an N-intein or a polynucleotide encoding the N-terminal extein; and (d) a C-terminal extein comprising a C-terminal fragment of the prime editor fusion protein and a C-intein, or a polynucleotide encoding the C-terminal extein; wherein the N-intein and the C-intein of the N-terminal and C-terminal exteins are capable of self-excision to join the N-terminal fragment and the C-terminal fragment to form the prime editor fusion protein, and wherein the prime editor fusion protein comprises a Cas9 nickase and a reverse transcriptase. In some embodiments, the Cas9 nickase comprises an amino acid sequence comprising at least 80%, 85%, 90%, 95%, 96%, 97%, 98%, 99%, or 100% identity to any one of SEQ ID NOs: 593, 594, 596, 597, 599, 600, 602, 603, 605, 606, 608, 609, 611, 612, 614, 615, 617, 618, or 619. In some embodiments, the reverse transcriptase comprises an amino acid sequence comprising at least 80%, 85%, 90%, 95%, 96%, 97%, 98%, 99%, or 100% identity to any one of SEQ ID NOs: 589, 590, or 591. In some embodiments, the sequence identities are determined by Needleman-Wunsch alignment of two protein sequences with Gap Costs set to Existence: 11 Extension: 1 where percent identity is calculated by dividing the number of identities by a length of the alignment.

In one aspect, provided herein is a prime editing system comprising: (a) a PEgRNA of the disclosure or any of the aspects herein, or the nucleotide encoding the PEgRNA; and (b) a prime editor comprising a Cas9 nickase comprising a nuclease inactivating mutation in the HNH domain, or a nucleic acid encoding the Cas9 nickase, and a reverse transcriptase, or a nucleic acid encoding the reverse transcriptase. In some embodiments, the Cas9 nickase comprises an amino acid sequence comprising at least 80%, 85%, 90%, 95%, 96%, 97%, 98%, 99%, or 100% identity to any one of SEQ ID NOs: 593, 594, 596, 597, 599, 600, 602, 603, 605, 606, 608, 609, 611, 612, 614, 615, 617, 618, or 619. In some embodiments, the reverse transcriptase comprises an amino acid sequence comprising at least 80%, 85%, 90%, 95%, 96%, 97%, 98%, 99%, or 100% identity to any one of SEQ ID NOs: 589, 590, or 591. In some embodiments, the sequence identities are determined by Needleman-Wunsch alignment of two protein sequences with Gap Costs set to Existence: 11 Extension: 1 where percent identity is calculated by dividing the number of identities by a length of the alignment.

In one aspect, provided herein is a prime editing system comprising: (a) a PEgRNA of the disclosure or any of the aspects herein or the nucleotide encoding the PEgRNA; (b) an N-terminal extein comprising an N-terminal fragment of a prime editor fusion protein and an N-intein or a polynucleotide encoding the N-terminal extein; and (c) a C-terminal extein comprising a C-terminal fragment of the prime editor fusion protein and a C-intein, or a polynucleotide encoding the C-terminal extein; wherein the N-intein and the C-intein of the N-terminal and C-terminal exteins are capable of self-excision to join the N-terminal fragment and the C-terminal fragment to form the prime editor fusion protein, and wherein the prime editor fusion protein comprises a Cas9 nickase and a reverse transcriptase. In some embodiments, the Cas9 nickase comprises an amino acid sequence comprising at least 80%, 85%, 90%, 95%, 96%, 97%, 98%, 99%, or 100% identity to any one of SEQ ID NOs: 593, 594, 596, 597, 599, 600, 602, 603, 605, 606, 608, 609, 611, 612, 614, 615, 617, 618, or 619. In some embodiments, the reverse transcriptase comprises an amino acid sequence comprising at least 80%, 85%, 90%, 95%, 96%, 97%, 98%, 99%, or 100% identity to any one of SEQ ID NOs: 589, 590, or 591. In some embodiments, the sequence identities are determined by Needleman-Wunsch alignment of two protein sequences with Gap Costs set to Existence: 11 Extension: 1 where percent identity is calculated by dividing the number of identities by a length of the alignment.

In one aspect, provided herein is a population of viral particles collectively comprising the one or more nucleic acids encoding the prime editing system of the disclosure or any of the aspects herein. In some embodiments, the viral particles are AAV particles.

In one aspect, provided herein is an LNP comprising the prime editing system of the disclosure or any of the aspects herein. In some embodiments, the LNP comprises the PEgRNA, the nucleic acid encoding the Cas9 nickase, and the nucleic acid encoding the reverse transcriptase. In some embodiments, the nucleic acid encoding the Cas9 nickase and the nucleic acid encoding the reverse transcriptase are mRNA. In some embodiments, the nucleic acid encoding the Cas9 nickase and the nucleic acid encoding the reverse transcriptase are the same molecule. In some embodiments, the Cas9 nickase comprises an amino acid sequence comprising at least 80%, 85%, 90%, 95%, 96%, 97%, 98%, 99%, or 100% identity to any one of SEQ ID NOs: 593, 594, 596, 597, 599, 600, 602, 603, 605, 606, 608, 609, 611, 612, 614, 615, 617, 618, or 619. In some embodiments, the reverse transcriptase comprises an amino acid sequence comprising at least 80%, 85%, 90%, 95%, 96%, 97%, 98%, 99%, or 100% identity to any one of SEQ ID NOs: 589, 590, or 591. In some embodiments, the sequence identities are determined by Needleman-Wunsch alignment of two protein sequences with Gap Costs set to Existence: 11 Extension: 1 where percent identity is calculated by dividing the number of identities by a length of the alignment.

In one aspect, provided herein is a method of correcting or editing a CLRN1 gene, the method comprising contacting the CLRN1 gene with: (a) the PEgRNA of the disclosure or any one of the aspects herein and a prime editor comprising a Cas9 nickase comprising a nuclease inactivating mutation in a HNH domain, and a reverse transcriptase; or (b) the prime editing system of the disclosure or any of the aspects herein. In some embodiments, the CLRN1 gene is in a cell. In some embodiments, the cell is a mammalian cell. In some embodiments, the cell is a human cell. In some embodiments, the cell is a primary cell. In some embodiments, the cell is in a subject. In some embodiments, the subject is a human. In some embodiments, the cell is from a subject having Usher Syndrome Type 3. In some embodiments, contacting the CLRN1 gene comprises contacting the cell with (i) a population of viral particles of the disclosure or any of the aspects herein; or (ii) a LNP of the disclosure or any of the aspects herein. In some embodiments, the Cas9 nickase comprises an amino acid sequence comprising at least 80%, 85%, 90%, 95%, 96%, 97%, 98%, 99%, or 100% identity to any one of SEQ ID NOs: 593, 594, 596, 597, 599, 600, 602, 603, 605, 606, 608, 609, 611, 612, 614, 615, 617, 618, or 619. In some embodiments, the reverse transcriptase comprises an amino acid sequence comprising at least 80%, 85%, 90%, 95%, 96%, 97%, 98%, 99%, or 100% identity to any one of SEQ ID NOs: 589, 590, or 591. In some embodiments, the sequence identities are determined by Needleman-Wunsch alignment of two protein sequences with Gap Costs set to Existence: 11 Extension: 1 where percent identity is calculated by dividing the number of identities by a length of the alignment.

In one aspect, provided herein is a method for treating Usher Syndrome Type 3 in a subject in need thereof, the method comprising administering to the subject: (a) a PEgRNA of the disclosure or any of the aspects herein and a prime editor comprising a Cas9 nickase comprising a nuclease inactivating mutation in the HNH domain and a reverse transcriptase; (b) a prime editing system of the disclosure or any of the aspects herein; (c) a population of viral particles of the disclosure or any of the aspects herein; or (d) a LNP of the disclosure or any of the aspects herein. In some embodiments, the Cas9 nickase comprises an amino acid sequence comprising at least 80%, 85%, 90%, 95%, 96%, 97%, 98%, 99%, or 100% identity to any one of SEQ ID NOs: 593, 594, 596, 597, 599, 600, 602, 603, 605, 606, 608, 609, 611, 612, 614, 615, 617, 618, or 619. In some embodiments, the reverse transcriptase comprises an amino acid sequence comprising at least 80%, 85%, 90%, 95%, 96%, 97%, 98%, 99%, or 100% identity to any one of SEQ ID NOs: 589, 590, or 591. In some embodiments, the sequence identities are determined by Needleman-Wunsch alignment of two protein sequences with Gap Costs set to Existence: 11 Extension: 1 where percent identity is calculated by dividing the number of identities by a length of the alignment.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 depicts a schematic of a prime editing guide RNA (PEgRNA) binding to a double stranded target DNA sequence.

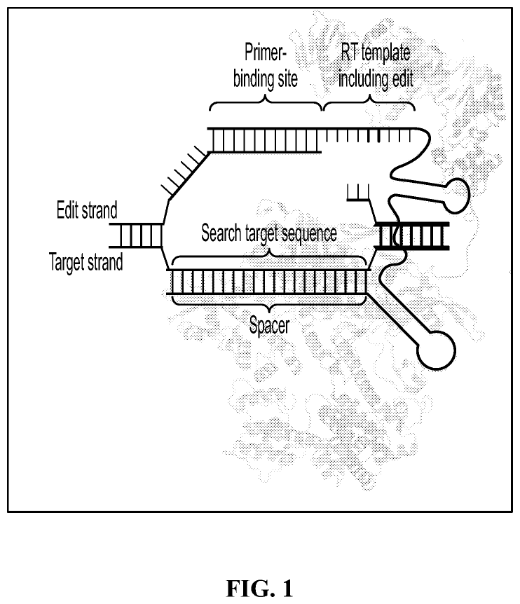

FIG. 2 depicts a PEgRNA architectural overview in an exemplary schematic of PEgRNA designed for a prime editor.

FIG. 3 is a schematic showing the spacer and gRNA core part of an exemplary guide RNA, in two separate molecules. The rest of the PEgRNA structure is not shown.

### DETAILED DESCRIPTION

Provided herein, in some embodiments, are compositions and methods to edit the target gene CLRN1 with prime editing. In certain embodiments, provided herein are compositions and methods for correction of mutations in the CLRN1 gene associated with Usher Syndrome type 3. Compositions provided herein can comprise prime editors (PEs) that may use engineered guide polynucleotides, e.g., prime editing guide RNAs (PEgRNAs), that can direct PEs to specific DNA targets and can encode DNA edits on the target gene CLRN1 that serve a variety of functions, including direct correction of disease-causing mutations.

The following description and examples illustrate embodiments of the present disclosure in detail. It is to be understood that this disclosure is not limited to the particular embodiments described herein and as such can vary. Those of skill in the art will recognize that there are numerous variations and modifications of this disclosure, which are encompassed within its scope. Although various features of the present disclosure can be described in the context of a single embodiment, the features can also be provided separately or in any suitable combination. Conversely, although the present disclosure can be described herein in the context of separate embodiments for clarity, the present disclosure can also be implemented in a single embodiment.

### Definitions

Unless defined otherwise, all technical and scientific terms used herein have the same meaning as is commonly understood by one of ordinary skill in the art.

The terminology used herein is for the purpose of describing particular embodiments only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof as used herein mean “comprising”.

Unless otherwise specified, the words “comprising”, “comprise”, “comprises”, “having”, “have”, “has”, “including”, “includes”, “include”, “containing”, “contains” and “contain” are inclusive or open-ended and do not exclude additional, unrecited elements or method steps.

Reference to “some embodiments”, “an embodiment”, “one embodiment”, or “other embodiments” means that a particular feature or characteristic described in connection with the embodiments is included in at least one or more embodiments, but not necessarily all embodiments, of the present disclosure.

The term “about” or “approximately” in relation to a numerical value means a range of values that fall within 10% greater than or 10% less than the value. For example, about x means x±(10% * x).

As used herein, a “cell” can generally refer to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant, an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), et cetera. Sometimes a cell may not originate from a natural organism (e.g., a cell can be synthetically made, sometimes termed an artificial cell).

In some embodiments, the cell is a human cell. A cell may be of or derived from different tissues, organs, and/or cell types. In some embodiments, the cell is a primary cell. In some embodiments, the term “primary cell” means a cell isolated from an organism, e.g., a mammal, which is grown in tissue culture (i.e., in vitro) for the first time before subdivision and transfer to a subculture. In some embodiments, the cell is a stem cell. In some non-limiting examples, mammalian cells, including primary cells and stem cells, can be modified through introduction of one or more polynucleotides, polypeptides, and/or prime editing compositions (e.g., through transfection, transduction, electroporation and the like) and further passaged. Such modified mammalian primary cells include retinal cells (e.g., photoreceptors, retinal pigment epithelium cells, Müller cells), epithelial cells (e.g., mammary epithelial cells, intestinal epithelial cells, hepatocytes), endothelial cells, glial cells, neural cells, hair cells, formed elements of the blood (e.g., lymphocytes, bone marrow cells), precursors of any of these somatic cell types, and stem cells. In some embodiments, the cell is a fibroblast. In some embodiments, the cell is a stem cell. In some embodiments, the cell is a pluripotent stem cell. In some embodiments, the cell is an induced pluripotent stem cell (iPSC). In some embodiments, the cell is a progenitor cell. In some embodiments, the cell is a human progenitor cell. In some embodiments, the cell is a tissue-specific stem cell or a mesenchymal stem cell. In some embodiments, the cell is a retinal progenitor cell. In some embodiments, the cell is a retinal precursor cell. In some embodiments, the cell is an embryonic stem cell (ESC). In some embodiments, the cell is a human stem cell. In some embodiments, the cell is a human pluripotent stem cell. In some embodiments, the cell is a human fibroblast. In some embodiments, the cell is an induced human pluripotent stem cell. In some embodiments, the cell is a human stem cell. In some embodiments, the cell is a human embryonic stem cell. In some embodiments, the cell is a human retinal progenitor cell. In some embodiments, the cell is a human retinal precursor cell. In some embodiments, the cell is in a subject, e.g., a human subject.

In some embodiments, a cell is not isolated from an organism but forms part of a tissue or organ of an organism, e.g., a mammal. In some non-limiting examples, mammalian cells include muscle cells (e.g., cardiac muscle cells, smooth muscle cells, myosatellite cells), epithelial cells (e.g., mammary epithelial cells, intestinal epithelial cells, hepatocytes), endothelial cells, glial cells, neural cells, formed elements of the blood (e.g., lymphocytes, bone marrow cells), precursors of any of these somatic cell types, and stem cells. In some embodiments, the cell is a pigmented epithelial cell. In some embodiments, the cell is a retinal cell. In some embodiments, the cell is a photoreceptor cell. In some embodiments, the cell is a hair cell. In some embodiments, the cell is an inner hair cell. In some embodiments, the cell is an outer hair cell. In some embodiments, the cell is a Müller cell. In some embodiments, the cell is a rod cell. In some embodiments, the cell is a cone cell. In some embodiments, the cell is a human stem cell.

In some embodiments, the cell is a differentiated cell. In some embodiments, cell is a fibroblast. In some embodiments, the cell is differentiated from an induced pluripotent stem cell. In some embodiments, the cell is a retinal cell, a pigmented epithelial cell, a rod cell, a cone cell, or a retinal ganglion differentiated from an iPSC, ESC, or a retinal progenitor cell.

In some embodiments, the cell is a differentiated human cell. In some embodiments, cell is a human fibroblast. In some embodiments, the cell is differentiated from an induced human pluripotent stem cell. In some embodiments, the cell is a retinal cell, a pigmented epithelial cell, a rod cell, a cone cell, or a retinal ganglion differentiated from a human iPSC, a human ESC, or a human retinal progenitor cell.

In some embodiments, the cell comprises a prime editor, a PEgRNA, or a prime editing composition disclosed herein. In some embodiments, the cell further comprises an ngRNA. In some embodiments, the cell is from a human subject. In some embodiments, the human subject has a disease or condition, or is at a risk of developing a disease or a condition, associated with a mutation to be corrected by prime editing, for example, Usher Syndrome type 3. In some embodiments, the cell is from a human subject, and comprises a prime editor, a PEgRNA, or a prime editing composition for correction of the mutation. In some embodiments, the cell is from the human subject and the mutation has been edited or corrected by prime editing. In some embodiments, the cell is in a human subject, and comprises a prime editor, a PEgRNA, or a prime editing composition for correction of the mutation. In some embodiments, the cell is from the human subject and the mutation has been edited or corrected by prime editing. In some embodiments, the cell is in a subject, e.g., a human subject. In some embodiments, the cell is obtained from a subject prior to editing. For example, the cell is obtained from a subject having a mutation in the CLRN1 gene.

The term “substantially” as used herein may refer to a value approaching 100% of a given value. In some embodiments, the term may refer to an amount that may be at least about 70%, 80%, 90%, 91%, 92%, 93%, 94%, 95%, 96%, 97%, 98%, 99%, 99.9%, or 99.99% of a total amount. In some embodiments, the term may refer to an amount that may be about 100% of a total amount.

The terms “protein” and “polypeptide” can be used interchangeably to refer to a polymer of two or more amino acids joined by covalent bonds (e.g., an amide bond) that can adopt a three-dimensional conformation. In some embodiments, a protein or polypeptide comprises at least 10 amino acids, 15 amino acids, 20 amino acids, 30 amino acids or 50 amino acids joined by covalent bonds (e.g., amide bonds). In some embodiments, a protein comprises at least two amide bonds. In some embodiments, a protein comprises multiple amide bonds. In some embodiments, a protein comprises an enzyme, enzyme precursor proteins, regulatory protein, structural protein, receptor, nucleic acid binding protein, a biomarker, a member of a specific binding pair (e.g., a ligand or aptamer), or an antibody. In some embodiments, a protein may be a full-length protein (e.g., a fully processed protein having certain biological function). In some embodiments, a protein may be a variant or a fragment of a full-length protein. For example, in some embodiments, a Cas9 protein domain comprises an H840A amino acid substitution compared to a naturally occurring S. pyogenes Cas9 protein. A variant of a protein or enzyme, for example a variant reverse transcriptase, comprises a polypeptide having an amino acid sequence that is about 60% identical, about 70% identical, about 80% identical, about 90% identical, about 95% identical, about 96% identical, about 97% identical, about 98% identical, about 99% identical, about 99.5% identical, or about 99.9% identical to the amino acid sequence of a reference protein.

In some embodiments, a protein comprises one or more protein domains or subdomains. As used herein, the term “polypeptide domain”, “protein domain”, or “domain” when used in the context of a protein or polypeptide, refers to a polypeptide chain that has one or more biological functions, e.g., a catalytic function, a protein-protein binding function, or a protein-DNA function. In some embodiments, a protein comprises multiple protein domains. In some embodiments, a protein comprises multiple protein domains that are naturally occurring. In some embodiments, a protein comprises multiple protein domains from different naturally occurring proteins. For example, in some embodiments, a prime editor may be a fusion protein comprising a Cas9 protein domain of S. pyogenes and a reverse transcriptase protein domain of a retrovirus (e.g., a Moloney murine leukemia virus) or a variant of the retrovirus. A protein that comprises amino acid sequences from different origins or naturally occurring proteins may be referred to as a fusion, or chimeric protein.

In some embodiments, a protein comprises a functional variant or functional fragment of a full-length wild type protein. A “functional fragment” or “functional portion”, as used herein, refers to any portion of a reference protein (e.g., a wild type protein) that encompasses less than the entire amino acid sequence of the reference protein while retaining one or more of the functions, e.g., catalytic or binding functions. For example, a functional fragment of a reverse transcriptase may encompass less than the entire amino acid sequence of a wild type reverse transcriptase, but retains the ability under at least one set of conditions to catalyze the polymerization of a polynucleotide. When the reference protein is a fusion of multiple functional domains, a functional fragment thereof may retain one or more of the functions of at least one of the functional domains. For example, a functional fragment of a Cas9 may encompass less than the entire amino acid sequence of a wild type Cas9 but retains its DNA binding ability and lacks its nuclease activity partially or completely.

A “functional variant” or “functional mutant”, as used herein, refers to any variant or mutant of a reference protein (e.g., a wild type protein) that encompasses one or more alterations to the amino acid sequence of the reference protein while retaining one or more of the functions, e.g., catalytic or binding functions. In some embodiments, the one or more alterations to the amino acid sequence comprises amino acid substitutions, insertions or deletions, or any combination thereof. In some embodiments, the one or more alterations to the amino acid sequence comprises amino acid substitutions. For example, a functional variant of a reverse transcriptase may comprise one or more amino acid substitutions compared to the amino acid sequence of a wild type reverse transcriptase, but retains the ability under at least one set of conditions to catalyze the polymerization of a polynucleotide. When the reference protein is a fusion of multiple functional domains, a functional variant thereof may retain one or more of the functions of at least one of the functional domains. For example, in some embodiments, a functional fragment of a Cas9 may comprise one or more amino acid substitutions in a nuclease domain, e.g., an H840A amino acid substitution, compared to the amino acid sequence of a wild type Cas9, but retains the DNA binding ability and lacks the nuclease activity partially or completely.

The term “function” and its grammatical equivalents as used herein may refer to a capability of operating, having, or serving an intended purpose. Functional may comprise any percent from baseline to 100% of an intended purpose. For example, functional may comprise or comprise about 5%, 10%, 15%, 20%, 25%, 30%, 35%, 40%, 45%, 50%, 55%, 60%, 65%, 70%, 75%, 80%, 85%, 90%, 95%, or up to about 100% of an intended purpose. In some embodiments, the term functional may mean over or over about 100% of normal function, for example, 125%, 150%, 175%, 200%, 250%, 300%, 400%, 500%, 600%, 700% or up to about 1000% of an intended purpose.

In some embodiments, a protein or polypeptides includes naturally occurring amino acids (e.g., one of the twenty amino acids commonly found in peptides synthesized in nature, and known by the one letter abbreviations A, R, N, C, D, Q, E, G, H, I, L, K, M, F, P, S, T, W, Y and V). In some embodiments, a protein or polypeptides includes non-naturally occurring amino acids (e.g., amino acids which is not one of the twenty amino acids commonly found in peptides synthesized in nature, including synthetic amino acids, amino acid analogs, and amino acid mimetics). In some embodiments, a protein or polypeptide is modified.

In some embodiments, a protein comprises an isolated polypeptide. The term “isolated” means free or removed to varying degrees from components which normally accompany it as found in the natural state or environment. For example, a polypeptide naturally present in a living animal is not isolated, and the same polypeptide partially or completely separated from the coexisting materials of its natural state is isolated.

In some embodiments, a protein is present within a cell, a tissue, an organ, or a virus particle. In some embodiments, a protein is present within a cell or a part of a cell (e.g., a bacteria cell, a plant cell, or an animal cell). In some embodiments, the cell is in a tissue, in a subject, or in a cell culture. In some embodiments, the cell is a microorganism (e.g., a bacterium, fungus, protozoan, or virus). In some embodiments, a protein is present in a mixture of analytes (e.g., a lysate). In some embodiments, the protein is present in a lysate from a plurality of cells or from a lysate of a single cell.

The terms “homologous,” “homology,” or “percent homology” as used herein refer to the degree of sequence identity between an amino acid and a corresponding reference amino acid sequence or a polynucleotide sequence and a corresponding reference polynucleotide sequence. “Homology” can refer to polymeric sequences, e.g., polypeptide or DNA sequences that are similar. Homology can mean, for example, nucleic acid sequences with at least about: 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity. In other embodiments, a “homologous sequence” of nucleic acid sequences may exhibit 93%, 95% or 98% sequence identity to the reference nucleic acid sequence. For example, a “region of homology to a genomic region” can be a region of DNA that has a similar sequence to a given genomic region in the genome. A region of homology can be of any length that is sufficient to promote binding of a spacer, a primer binding site, or protospacer sequence to the genomic region. For example, the region of homology can comprise at least 5, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100 or more bases in length such that the region of homology has sufficient homology to undergo binding with the corresponding genomic region.

When a percentage of sequence homology or identity is specified, in the context of two nucleic acid sequences or two polypeptide sequences, the percentage of homology or identity generally refers to the alignment of two or more sequences across a portion of their length when compared and aligned for maximum correspondence. When a position in the compared sequence can be occupied by the same base or amino acid, then the molecules can be homologous at that position. Unless stated otherwise, sequence homology or identity is assessed over the specified length of the nucleic acid, polypeptide or portion thereof. In some embodiments, the homology or identity is assessed over a functional portion or specified portion of the length.

Alignment of sequences for assessment of sequence homology can be conducted by algorithms known in the art, such as the Basic Local Alignment Search Tool (BLAST) algorithm, which is described in Altschul et al, J. Mol. Biol. 215:403-410, 1990. A publicly available, internet interface, for performing BLAST analyses is accessible through the National Center for Biotechnology Information. Additional known algorithms include those published in: Smith & Waterman, “Comparison of Biosequences”, Adv. Appl. Math. 2:482, 1981; Needleman & Wunsch, “A general method applicable to the search for similarities in the amino acid sequence of two proteins” J. Mol. Biol. 48:443, 1970; Pearson & Lipman “Improved tools for biological sequence comparison”, Proc. Natl. Acad. Sci. USA 85:2444, 1988; or by automated implementation of these or similar algorithms. Global alignment programs may also be used to align similar sequences of roughly equal size. Examples of global alignment programs include NEEDLE (available at www.ebi.ac.uk/Tools/psa/emboss_needle/) which is part of the EMBOSS package (Rice P et al., Trends Genet., 2000; 16: 276-277), and the GGSEARCH program https://fasta.bioch.virginia.edu/fasta_www2/, which is part of the FASTA package (Pearson W and Lipman D, 1988, Proc. Natl. Acad. Sci. USA, 85: 2444-2448). Both of these programs are based on the Needleman-Wunsch algorithm which is used to find the optimum alignment (including gaps) of two sequences along their entire length. A detailed discussion of sequence analysis can also be found in Unit 19.3 of Ausubel et al (“Current Protocols in Molecular Biology” John Wiley & Sons Inc, 1994-1998, Chapter 15, 1998). In some embodiments, alignment between a query sequence and a reference sequence is performed with Needleman-Wunsch alignment with Gap Costs set to Existence: 11 Extension: 1 where percent identity is calculated by dividing the number of identities by the length of the alignment, as further described in Altschul et al. (“Gapped BLAST and PSI-BLAST: a new generation of protein database search programs”, Nucleic Acids Res. 25:3389-3402, 1997) and Altschul et al, (“Protein database searches using compositionally adjusted substitution matrices”, FEBS J. 272:5101-5109, 2005).

A skilled person understands that amino acid (or nucleotide) positions may be determined in homologous sequences based on alignment, for example, “H840” in a reference Cas9 sequence may correspond to H839, or another position in a Cas9 homolog.

The term “polynucleotide” or “nucleic acid molecule” can be any polymeric form of nucleotides, including DNA, RNA, a hybridization thereof, or RNA-DNA chimeric molecules. In some embodiments, a polynucleotide comprises cDNA, genomic DNA, mRNA, tRNA, rRNA, or microRNA. In some embodiments, a polynucleotide is double stranded, e.g., a double-stranded DNA in a gene. In some embodiments, a polynucleotide is single-stranded or substantially single-stranded, e.g., single-stranded DNA or an mRNA. In some embodiments, a polynucleotide is a cell-free nucleic acid molecule. In some embodiments, a polynucleotide circulates in blood. In some embodiments, a polynucleotide is a cellular nucleic acid molecule. In some embodiments, a polynucleotide is a cellular nucleic acid molecule in a cell circulating in blood.

Polynucleotides can have any three-dimensional structure. The following are nonlimiting examples of polynucleotides: a gene or gene fragment (for example, a probe, primer, EST or SAGE tag), an exon, an intron, intergenic DNA (including, without limitation, heterochromatic DNA), messenger RNA (mRNA), transfer RNA (tRNA), ribosomal RNA (rRNA), a ribozyme, cDNA, a recombinant polynucleotide, a branched polynucleotide, a plasmid, a vector, isolated DNA, isolated RNA, sgRNA, guide RNA, a nucleic acid probe, a primer, an snRNA, a long non-coding RNA, a snoRNA, a siRNA, a miRNA, a tRNA-derived small RNA (tsRNA), an antisense RNA, an shRNA, or a small rDNA-derived RNA (srRNA).

In some embodiments, a polynucleotide comprises deoxyribonucleotides, ribonucleotides or analogs thereof. In some embodiments, a polynucleotide comprises modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component.

In some embodiments, a polynucleotide is composed of a specific sequence of four nucleotide bases: adenine (A); cytosine (C); guanine (G); thymine (T); and uracil (U) for thymine when the polynucleotide is RNA. In some embodiments, the polynucleotide may comprise one or more other nucleotide bases, such as inosine (I), which is read by the translation machinery as guanine (G).

In some embodiments, a polynucleotide may be modified. As used herein, the terms “modified” or “modification” refers to chemical modification with respect to the A, C, G, T and U nucleotides. In some embodiments, modifications may be on the nucleoside base and/or sugar portion of the nucleosides that comprise the polynucleotide. In some embodiments, the modification may be on the internucleoside linkage (e.g., phosphate backbone). In some embodiments, multiple modifications are included in the modified nucleic acid molecule. In some embodiments, a single modification is included in the modified nucleic acid molecule.

The term “complement”, “complementary”, or “complementarity” as used herein, refers to the ability of two polynucleotide molecules to base pair with each other. Complementary polynucleotides may base pair via hydrogen bonding, which may be Watson Crick, Hoogsteen or reversed Hoogsteen hydrogen bonding. For example, an adenine on one polynucleotide molecule will base pair to a thymine or an uracil on a second polynucleotide molecule and a cytosine on one polynucleotide molecule will base pair to a guanine on a second polynucleotide molecule. Two polynucleotide molecules are complementary to each other when a first polynucleotide molecule comprising a first nucleotide sequence can base pair with a second polynucleotide molecule comprising a second nucleotide sequence. For instance, the two DNA molecules 5′-ATGC-3′ and 5′-GCAT-3′ are complementary, and the complement of the DNA molecule 5′-ATGC-3′ is 5′-GCAT-3′. A percentage of complementarity indicates the percentage of nucleotides in a polynucleotide molecule which can base pair with a second polynucleotide molecule (e.g., 5, 6, 7, 8, 9, 10 out of 10 being 50%, 60%, 70%, 80%, 90%, and 100% complementary, respectively). “Perfectly complementary” means that all the contiguous nucleotides of a polynucleotide molecule will base pair with the same number of contiguous nucleotides in a second polynucleotide molecule. “Substantially complementary” as used herein refers to a degree of complementarity that can be 70%, 75%, 80%, 85%, 90%, 95%, 97%, 98%, or 99% over all or a portion of two polynucleotide molecules. In some embodiments, the portion of complementarity may be a region of 10, 15, 20, 25, 30, 35, 40, 45, 50, or more nucleotides. “Substantial complementary” can also refer to a 100% complementarity over a portion or a region of two polynucleotide molecules. In some embodiments, the portion or the region of complementarity between the two polynucleotide molecules is at least 60%, 65%, 70%, 75%, 80%, 85%, 90%, 95%, 97%, 98%, or 99% of the length of at least one of the two polynucleotide molecules or a functional or defined portion thereof.

As used herein, “expression” refers to the process by which polynucleotides are transcribed into mRNA and/or the process by which polynucleotides, e.g., the transcribed mRNA, translated into peptides, polypeptides, or proteins. If the polynucleotide is derived from genomic DNA, expression may include splicing of the mRNA in a eukaryotic cell. In some embodiments, expression of a polynucleotide, e.g., a gene or a DNA encoding a protein, is determined by the amount of the protein encoded by the gene after transcription and translation of the gene. In some embodiments, expression of a polynucleotide, e.g., a gene or a DNA encoding a protein, is determined by the amount of a functional form of the protein encoded by the gene after transcription and translation of the gene. In some embodiments, expression of a gene is determined by the amount of the mRNA, or transcript, that is encoded by the gene after transcription the gene. In some embodiments, expression of a polynucleotide, e.g., an mRNA, is determined by the amount of the protein encoded by the mRNA after translation of the mRNA. In some embodiments, expression of a polynucleotide, e.g., a mRNA or coding RNA, is determined by the amount of a functional form of the protein encoded by the polypeptide after translation of the polynucleotide.

The terms “equivalent” or “biological equivalent” are used interchangeably when referring to a particular molecule, or biological or cellular material, and means a molecule having minimal homology to another molecule while still maintaining a desired structure or functionality.

The term “encode” as it is applied to polynucleotides refers to a polynucleotide which is said to “encode” another polynucleotide, a polypeptide, or an amino acid if, in its native state or when manipulated by methods well known to those skilled in the art, it can be used as polynucleotide synthesis template, e.g., transcribed into an RNA, reverse transcribed into a DNA or cDNA, and/or translated to produce an amino acid, or a polypeptide or fragment thereof. In some embodiments, a polynucleotide comprising three contiguous nucleotides form a codon that encodes a specific amino acid. In some embodiments, a polynucleotide comprises one or more codons that encode a polypeptide. In some embodiments, a polynucleotide comprising one or more codons comprises a mutation in a codon compared to a wild-type reference polynucleotide. In some embodiments, the mutation in the codon encodes an amino acid substitution in a polypeptide encoded by the polynucleotide as compared to a wild-type reference polypeptide.

The term “mutation” as used herein refers to a change and/or alteration in an amino acid sequence of a protein or nucleic acid sequence of a polynucleotide. Such changes and/or alterations may comprise the substitution, insertion, deletion and/or truncation of one or more amino acids, in the case of an amino acid sequence, and/or nucleotides, in the case of nucleic acid sequence, compared to a reference amino acid or a reference nucleic acid sequence. In some embodiments, the reference sequence is a wild-type sequence. In some embodiments, a mutation in a nucleic acid sequence of a polynucleotide encodes a mutation in the amino acid sequence of a polypeptide. In some embodiments, the mutation in the amino acid sequence of the polypeptide or the mutation in the nucleic acid sequence of the polynucleotide is a mutation associated with a disease state.

The term “subject” and its grammatical equivalents as used herein may refer to a human or a non-human. A subject may be a mammal. A human subject may be male or female. A human subject may be of any age. A subject may be a human embryo. A human subject may be a newborn, an infant, a child, an adolescent, or an adult. A human subject may be in need of treatment for a genetic disease or disorder.

The terms “treatment” or “treating” and their grammatical equivalents may refer to the medical management of a subject with an intent to cure, ameliorate, or ameliorate a symptom of, a disease, condition, or disorder. Treatment may include active treatment, that is, treatment directed specifically toward the improvement of a disease, condition, or disorder. Treatment may include causal treatment, that is, treatment directed toward removal of the cause of the associated disease, condition, or disorder. In addition, this treatment may include palliative treatment, that is, treatment designed for the relief of symptoms rather than the curing of the disease, condition, or disorder. Treatment may include supportive treatment, that is, treatment employed to supplement another specific therapy directed toward the improvement of the disease, condition, or disorder. In some embodiments, a condition may be pathological. In some embodiments, a treatment may not completely cure or prevent a disease, condition, or disorder. In some embodiments, a treatment ameliorates, but does not completely cure or prevent a disease, condition, or disorder. In some embodiments, a subject may be treated for 12 hours, 24 hours, 2 days, 3 days, 4 days, 5 days, 6 days, 7 days, 2 weeks, 3 weeks, 4 weeks, 2 months, 3 months, 4 months, 5 months, 6 months, 1 year, 2 years, 3 years, 4 years, 5 years, 6 years, indefinitely, or life of the subject.

The term “ameliorate” and its grammatical equivalents means to decrease, suppress, attenuate, diminish, arrest, or stabilize the development or progression of a disease.

The terms “prevent” or “preventing” means delaying, forestalling, or avoiding the onset or development of a disease, condition, or disorder for a period of time. Prevent also means reducing risk of developing a disease, disorder, or condition. Prevention includes minimizing or partially or completely inhibiting the development of a disease, condition, or disorder. In some embodiments, a composition, e.g., a pharmaceutical composition, prevents a disorder by delaying the onset of the disorder for 12 hours, 24 hours, 2 days, 3 days, 4 days, 5 days, 6 days, 7 days, 2 weeks, 3 weeks, 4 weeks, 2 months, 3 months, 4 months, 5 months, 6 months, 1 year, 2 years, 3 years, 4 years, 5 years, 6 years, indefinitely, or life of a subject.

The term “effective amount” or “therapeutically effective amount” refers to a quantity of a composition, for example a prime editing composition comprising a construct, that can be sufficient to result in a desired activity upon introduction into a subject as disclosed herein. An effective amount of the prime editing compositions can be provided to the target gene or cell, whether the cell is ex vivo or in vivo.

An effective amount can be the amount to induce, for example, at least about a 2-fold change (increase or decrease) or more in the amount of target nucleic acid modulation (e.g., expression of CLRN1 gene to produce functional CLRN1 clarin-1 protein) observed relative to a negative control. An effective amount or dose can induce, for example, about 2-fold increase, about 3-fold increase, about 4-fold increase, about 5-fold increase, about 6-fold increase, about 7-fold increase, about 8-fold increase, about 9-fold increase, about 10-fold increase, about 25-fold increase, about 50-fold increase, about 100-fold increase, about 200-fold increase, about 500-fold increase, about 700-fold increase, about 1000-fold increase, about 5000-fold increase, or about 10,000-fold increase in target gene modulation (e.g., expression of a target CLRN1 gene to produce functional clarin-1).

The amount of target gene modulation may be measured by any suitable method known in the art. In some embodiments, the “effective amount” or “therapeutically effective amount” is the amount of a composition that is required to ameliorate the symptoms of a disease relative to an untreated patient. In some embodiments, an effective amount is the amount of a composition sufficient to introduce an alteration in a gene of interest in a cell (e.g., a cell in vitro or in vivo).

In some embodiments, an effective amount can be an amount to induce, when administered to a population of cells, at least about 2-fold increase, about 3-fold increase, about 4-fold increase, about 5-fold increase, about 6-fold increase, about 7-fold increase, about 8-fold increase, about 9-fold increase, about 10-fold increase, about 25-fold increase, about 50-fold increase, about 100-fold increase, about 200-fold increase, about 500-fold increase, about 700-fold increase, about 1000-fold increase, about 5000-fold increase, or about 10,000-fold increase in the number of cells that have an intended nucleotide edit, for example, a nucleotide edit that corrects a c. 144 T->G (encoding N48K amino acid substitution) mutation in the CLRN1 gene. In some embodiments, an effective amount can be an amount to induce, when administered to a population of cells, a certain percentage of the population of cells to have a correction of a mutation, for example, an N48K mutation in CLRN] gene or one or more other mutations in CLRN] gene. For example, in some embodiments, an effective amount can be the amount to induce, when administered to or introduced to a population of cells, installation of one or more intended nucleotide edits that correct a mutation, for example, c. 144 T->G (encoding N48K amino acid substitution) mutation in the CLRN1 gene, in at least about 1%, 2%, 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99% of the population of cells.

### Prime Editing

The term “prime editing” refers to programmable editing of a target DNA using a prime editor complexed with a PEgRNA to incorporate an intended nucleotide edit (also referred to herein as a nucleotide change) into the target DNA through target-primed DNA synthesis. A target gene of prime editing may comprise a double stranded DNA molecule having two complementary strands: a first strand that may be referred to as a “target strand” or a “non-edit strand”, and a second strand that may be referred to as a “non-target strand,” or an “edit strand.” In some embodiments, in a prime editing guide RNA (PEgRNA), a spacer sequence is complementary or substantially complementary to a specific sequence on the target strand, which may be referred to as a “search target sequence”. In some embodiments, the spacer sequence anneals with the target strand at the search target sequence. The target strand may also be referred to as the “non-Protospacer Adjacent Motif (non-PAM strand).” In some embodiments, the non-target strand may also be referred to as the “PAM strand”. In some embodiments, the PAM strand comprises a protospacer sequence and optionally a protospacer adjacent motif (PAM) sequence. In prime editing using a Cas-protein-based prime editor, a PAM sequence refers to a short DNA sequence immediately adjacent to the protospacer sequence on the PAM strand of the target gene. A PAM sequence may be specifically recognized by a programmable DNA binding protein, e.g., a Cas nickase or a Cas nuclease. In some embodiments, a specific PAM is characteristic of a specific programmable DNA binding protein, e.g., a Cas nickase or a Cas nuclease. A protospacer sequence refers to a specific sequence in the PAM strand of the target gene that is complementary to the search target sequence. In a PEgRNA, a spacer sequence may have a substantially identical sequence as the protospacer sequence on the edit strand of a target gene, except that the spacer sequence may comprise Uracil (U) and the protospacer sequence may comprise Thymine (T).

In some embodiments, the double stranded target DNA comprises a nick site on the PAM strand (or non-target strand). As used herein, a “nick site” refers to a specific position in between two nucleotides or two base pairs of the double stranded target DNA. In some embodiments, the position of a nick site is determined relative to the position of a specific PAM sequence. In some embodiments, the nick site is the particular position where a nick will occur when the double stranded target DNA is contacted with a nickase, for example, a Cas nickase, that recognizes a specific PAM sequence. In some embodiments, the nick site is upstream of a specific PAM sequence on the PAM strand of the double stranded target DNA. In some embodiments, the nick site is upstream of a PAM sequence recognized by a Cas9 nickase, wherein the Cas9 nickase comprises a nuclease active RuvC domain and a nuclease inactive HNH domain. In some embodiments, the nick site is downstream of a specific PAM sequence on the PAM strand of the double stranded target DNA. In some embodiments, the nick site is 3 nucleotides upstream of the PAM sequence, and the PAM sequence is recognized by a Streptococcus pyogenes Cas9 nickase, a P. lavamentivorans Cas9 nickase, a C. diphtheriae Cas9 nickase, a N. cinerea Cas9, a S. aureus Cas9, or a N. lari Cas9 nickase. In some embodiments, the nick site is 3 nucleotides upstream of the PAM sequence, and the PAM sequence is recognized by a Cas9 nickase, wherein the Cas9 nickase comprises a nuclease active RuvC domain and a nuclease inactive HNH domain. In some embodiments, the nick site is 2 nucleotides upstream of the PAM sequence, and the PAM sequence is recognized by a S. thermophilus Cas9 nickase that comprises a nuclease active RuvC domain and a nuclease inactive HNH domain.

A “primer binding site” (also referred to as PBS or primer binding site sequence) is a single-stranded portion of the PEgRNA that comprises a region of complementarity to the PAM strand (i.e. the non-target strand or the edit strand). The PBS is complementary or substantially complementary to a sequence on the PAM strand of the double stranded target DNA that is immediately upstream of the nick site. In some embodiments, in the process of prime editing, the PEgRNA complexes with and directs a prime editor to bind the search target sequence on the target strand of the double stranded target DNA, and generates a nick at the nick site on the non-target strand of the double stranded target DNA. In some embodiments, the PBS is complementary to or substantially complementary to, and can anneal to, a free 3′ end on the non-target strand of the double stranded target DNA at the nick site. In some embodiments, the PBS annealed to the free 3′ end on the non-target strand can initiate target-primed DNA synthesis.

An “editing template” of a PEgRNA is a single-stranded portion of the PEgRNA that is 5′ of the PBS and which encodes a single strand of DNA. The editing template may comprise a region of complementarity to the PAM strand (i.e. the non-target strand or the edit strand), and comprises one or more intended nucleotide edits compared to the endogenous sequence of the double stranded target DNA. In some embodiments, the editing template and the PBS are immediately adjacent to each other. Accordingly, in some embodiments, a PEgRNA in prime editing comprises a single-stranded portion that comprises the PBS and the editing template immediately adjacent to each other. In some embodiments, the single stranded portion of the PEgRNA comprising both the PBS and the editing template is complementary or substantially complementary to an endogenous sequence on the PAM strand (i.e. the non-target strand or the edit strand) of the double stranded target DNA except for one or more non-complementary nucleotides at the intended nucleotide edit positions. As used herein, regardless of relative 5′-3′ positioning in other context, the relative positions as between the PBS and the editing template, and the relative positions as among elements of a PEgRNA, are determined by the 5′ to 3′ order of the PEgRNA as a single molecule regardless of the position of sequences in the double stranded target DNA that may have complementarity or identity to elements of the PEgRNA. In some embodiments, the editing template is complementary or substantially complementary to a sequence on the PAM strand that is immediately downstream of the nick site, except for one or more non-complementary nucleotides at the intended nucleotide edit positions. The endogenous, e.g., genomic, sequence that is complementary or substantially complementary to the editing template, except for the one or more non-complementary nucleotides at the position corresponding to the intended nucleotide edit, may be referred to as an “editing target sequence”. In some embodiments, the editing template has identity or substantial identity to a sequence on the target strand that is complementary to, or having the same position in the genome as, the editing target sequence, except for one or more insertions, deletions, or substitutions at the intended nucleotide edit positions. In some embodiments, the editing template encodes a single stranded DNA, wherein the single stranded DNA has identity or substantial identity to the editing target sequence except for one or more insertions, deletions, or substitutions at the positions of the one or more intended nucleotide edits. In some embodiments, the editing template may encode the wild-type or non-disease associated gene sequence (or its complement if the edit strand is the antisense strand of a gene). In some embodiments, the editing template may encode the wild-type or non-disease associated protein, but contain one or more synonymous mutations relative to the wild-type or non-disease associated protein coding region. Such synonymous mutations may include, for example, mutations that decrease the ability of a PEgRNA to rebind to the same target sequence once the desired edit is installed in the genome (e.g., synonymous mutations that silence the endogenous PAM sequence or that edit the endogenous protospacer).

In some embodiments, a PEgRNA complexes with and directs a prime editor to bind to the search target sequence of the target gene. In some embodiments, the bound prime editor generates a nick on the edit strand (PAM strand) of the target gene at the nick site. In some embodiments, a primer binding site (PBS) of the PEgRNA anneals with a free 3′ end formed at the nick site, and the prime editor initiates DNA synthesis from the nick site, using the free 3′ end as a primer. Subsequently, a single-stranded DNA encoded by the editing template of the PEgRNA is synthesized. In some embodiments, the newly synthesized single-stranded DNA comprises one or more intended nucleotide edits compared to an endogenous target gene sequence. Accordingly, in some embodiments, the editing template of a PEgRNA is complementary to a sequence in the edit strand except for one or more mismatches at the intended nucleotide edit positions in the editing template. The endogenous, e.g., genomic, sequence that is partially complementary to the editing template may be referred to as an “editing target sequence”. Accordingly, in some embodiments, the new,ly synthesized single stranded DNA has identity or substantial identity to a sequence in the editing target sequence, except for one or more insertions, deletions, or substitutions at the intended nucleotide edit positions. In some embodiments, the editing template comprises at least 4 contiguous nucleotides of complementarity with the edit strand wherein the at least 4 nucleotides contiguous are located upstream of the 5′ most edit in the editing template.

In some embodiments, the newly synthesized single-stranded DNA equilibrates with the editing target on the edit strand of the target gene for pairing with the target strand of the target gene. In some embodiments, the editing target sequence of the target gene is excised by a flap endonuclease (FEN), for example, FEN1. In some embodiments, the FEN is an endogenous FEN, for example, in a cell comprising the target gene. In some embodiments, the FEN is provided as part of the prime editor, either linked to other components of the prime editor or provided in trans. In some embodiments, the newly synthesized single stranded DNA, which comprises the intended nucleotide edit, replaces the endogenous single stranded editing target sequence on the edit strand of the target gene. In some embodiments, the newly synthesized single stranded DNA and the endogenous DNA on the target strand form a heteroduplex DNA structure at the region corresponding to the editing target sequence of the target gene. In some embodiments, the newly synthesized single-stranded DNA comprising the nucleotide edit is paired in the heteroduplex with the target strand of the target DNA that does not comprise the nucleotide edit, thereby creating a mismatch between the two otherwise complementary strands. In some embodiments, the mismatch is recognized by DNA repair machinery, e.g., an endogenous DNA repair machinery. In some embodiments, through DNA repair, the intended nucleotide edit is incorporated into the target gene.

### Prime Editor

The term “prime editor (PE)” refers to the polypeptide or polypeptide components involved in prime editing, or any polynucleotide(s) encoding the polypeptide or polypeptide components. In various embodiments, a prime editor includes a polypeptide domain having DNA binding activity and a polypeptide domain having DNA polymerase activity. In some embodiments, the prime editor further comprises a polypeptide domain having nuclease activity. In some embodiments, the polypeptide domain having DNA binding activity comprises a nuclease domain or nuclease activity. In some embodiments, the polypeptide domain having nuclease activity comprises a nickase, or a fully active nuclease. As used herein, the term “nickase” refers to a nuclease capable of cleaving only one strand of a double-stranded DNA target. In some embodiments, the prime editor comprises a polypeptide domain that is an inactive nuclease. In some embodiments, the polypeptide domain having programmable DNA binding activity comprises a nucleic acid guided DNA binding domain, for example, a CRISPR-Cas protein, for example, a Cas9 nickase, a Cpf1 nickase, or another CRISPR-Cas nuclease. In some embodiments, the polypeptide domain having DNA polymerase activity comprises a template-dependent DNA polymerase, for example, a DNA-dependent DNA polymerase or an RNA-dependent DNA polymerase. In some embodiments, the DNA polymerase is a reverse transcriptase. In some embodiments, the prime editor comprises additional polypeptides involved in prime editing, for example, a polypeptide domain having 5′ endonuclease activity, e.g., a 5′ endogenous DNA flap endonucleases (e.g., FEN1), for helping to drive the prime editing process towards the edited product formation. In some embodiments, the prime editor further comprises an RNA-protein recruitment polypeptide, for example, a MS2 coat protein.

A prime editor may be engineered. In some embodiments, the polypeptide components of a prime editor do not naturally occur in the same organism or cellular environment. In some embodiments, the polypeptide components of a prime editor may be of different origins or from different organisms. In some embodiments, a prime editor comprises a DNA binding domain and a DNA polymerase domain that are derived from different species. In some embodiments, a prime editor comprises a Cas polypeptide (illustrative DNA binding domain) and a reverse transcriptase polypeptide (illustrative DNA polymerase domain) that are derived from different species. For example, a prime editor may comprise a S. pyogenes Cas9 polypeptide and a Moloney murine leukemia virus (M-MLV) reverse transcriptase polypeptide.

In some embodiments, polypeptide domains of a prime editor may be fused or linked by a peptide linker to form a fusion protein. In other embodiments, a prime editor comprises one or more polypeptide domains provided in trans as separate proteins, which are capable of being associated to each other through non-peptide linkages or through aptamers or recruitment sequences. For example, a prime editor may comprise a DNA binding domain and a reverse transcriptase domain associated with each other by an RNA-protein recruitment aptamer, e.g., a MS2 aptamer, which may be linked to a PEgRNA. Prime editor polypeptide components may be encoded by one or more polynucleotides in whole or in part. In some embodiments, a single polynucleotide, construct, or vector encodes the prime editor fusion protein. In some embodiments, multiple polynucleotides, constructs, or vectors each encode a polypeptide domain or portion of a domain of a prime editor, or a portion of a prime editor fusion protein. For example, a prime editor fusion protein may comprise an N-terminal portion fused to an intein-N and a C-terminal portion fused to an intein-C, each of which is individually encoded by an AAV vector.

### Prime Editor Nucleotide Polymerase Domain

In some embodiments, a prime editor comprises a nucleotide polymerase domain, e.g., a DNA polymerase domain. The DNA polymerase domain may be a wild-type DNA polymerase domain, a full-length DNA polymerase protein domain, or may be a functional mutant, a functional variant, or a functional fragment thereof. In some embodiments, the polymerase domain is a template dependent polymerase domain. For example, the DNA polymerase may rely on a template polynucleotide strand, e.g., the editing template sequence, for new strand DNA synthesis. In some embodiments, the prime editor comprises a DNA-dependent DNA polymerase. For example, a prime editor having a DNA-dependent DNA polymerase can synthesize a new single stranded DNA using a PEgRNA editing template that comprises a DNA sequence as a template. In such cases, the PEgRNA is a chimeric or hybrid PEgRNA, and comprising an extension arm comprising a DNA strand. The chimeric or hybrid PEgRNA may comprise an RNA portion (including the spacer and the gRNA core) and a DNA portion (the extension arm comprising the editing template that includes a strand of DNA).

In some embodiments, the DNA polymerases can be wild type polymerases from eukaryotic, prokaryotic, archaeal, or viral organisms, and/or the polymerases may be modified by genetic engineering, mutagenesis, or directed evolution-based processes. The polymerases can be a T7 DNA polymerase, T5 DNA polymerase, T4 DNA polymerase, Klenow fragment DNA polymerase, DNA polymerase III and the like. The polymerases can be thermostable, and can include Taq, Tne, Tma, Pfu, Tfl, Tth, Stoffel fragment, VENT® and DEEPVENT® DNA polymerases, KOD, Tgo, JDF3, and mutants, variants and derivatives thereof.

In some embodiments, the DNA polymerase is a bacteriophage polymerase, for example, a T4, T7, or phi29 DNA polymerase. In some embodiments, the DNA polymerase is an archaeal polymerase, for example, pol I type archaeal polymerase or a pol II type archaeal polymerase. In some embodiments, the DNA polymerase comprises a thermostable archaeal DNA polymerase. In some embodiments, the DNA polymerase comprises a eubacterial DNA polymerase, for example, Pol I, Pol II, or Pol III polymerase. In some embodiments, the DNA polymerase is a Pol I family DNA polymerase. In some embodiments, the DNA polymerase is a E. coli Pol I DNA polymerase. In some embodiments, the DNA polymerase is a Pol II family DNA polymerase. In some embodiments, the DNA polymerase is a Pyrococcusfuriosus (Pfu) Pol II DNA polymerase. In some embodiments, the DNA Polymerase is a Pol IV family DNA polymerase. In some embodiments, the DNA polymerase is a E. coli Pol IV DNA polymerase.

In some embodiments, the DNA polymerase comprises a eukaryotic DNA polymerase. In some embodiments, the DNA polymerase is a Pol-beta DNA polymerase, a Pol-lambda DNA polymerase, a Pol-sigma DNA polymerase, or a Pol-mu DNA polymerase. In some embodiments, the DNA polymerase is a Pol-alpha DNA polymerase. In some embodiments, the DNA polymerase is a POLA1 DNA polymerase. In some embodiments, the DNA polymerase is a POLA2 DNA polymerase. In some embodiments, the DNA polymerase is a Pol-delta DNA polymerase. In some embodiments, the DNA polymerase is a POLD1 DNA polymerase. In some embodiments, the DNA polymerase is a POLD2 DNA polymerase. In some embodiments, the DNA polymerase is a human POLD1 DNA polymerase. In some embodiments, the DNA polymerase is a human POLD2 DNA polymerase. In some embodiments, the DNA polymerase is a POLD3 DNA polymerase. In some embodiments, the DNA polymerase is a POLD4 DNA polymerase. In some embodiments, the DNA polymerase is a Pol-epsilon DNA polymerase. In some embodiments, the DNA polymerase is a POLE1 DNA polymerase. In some embodiments, the DNA polymerase is a POLE2 DNA polymerase. In some embodiments, the DNA polymerase is a POLE3 DNA polymerase. In some embodiments, the DNA polymerase is a Pol-eta (POLH) DNA polymerase. In some embodiments, the DNA polymerase is a Pol-iota (POLI) DNA polymerase. In some embodiments, the DNA polymerase is a Pol-kappa (POLK) DNA polymerase. In some embodiments, the DNA polymerase is a RevI DNA polymerase. In some embodiments, the DNA polymerase is a human RevI DNA polymerase. In some embodiments, the DNA polymerase is a viral DNA-dependent DNA polymerase. In some embodiments, the DNA polymerase is a B family DNA polymerase. In some embodiments, the DNA polymerase is a herpes simplex virus (HSV) UL30 DNA polymerase. In some embodiments, the DNA polymerase is a cytomegalovirus (CMV) UL54 DNA polymerase.

In some embodiments, the DNA polymerase is an archaeal polymerase. In some embodiments, the DNA polymerase is a Family B/pol I type DNA polymerase. For example, in some embodiments, the DNA polymerase is a homolog of Pfu from Pyrococcusfuriosus. In some embodiments, the DNA polymerase is a pol II type DNA polymerase. For example, in some embodiments, the DNA polymerase is a homolog of P. furiosus DP1/DP2.2-subunit polymerase. In some embodiments, the DNA polymerase lacks 5′ to 3′ nuclease activity. Suitable DNA polymerases (pol I or pol II) can be derived from archaea with optimal growth temperatures that are similar to the desired assay temperatures.

In some embodiments, the DNA polymerase comprises a thermostable archaeal DNA polymerase. In some embodiments, the thermostable DNA polymerase is isolated or derived from Pyrococcus species (furiosus, species GB-D, woesii, abysii, horikoshii), Thermococcus species (kodakaraensis KOD1, litoralis, species 9 degrees North-7, species JDF-3, gorgonarius), Pyrodictium occultum, and Archaeoglobus fulgidus.

Polymerases may also be from eubacterial species. In some embodiments, the DNA polymerase is a Pol I family DNA polymerase. In some embodiments, the DNA polymerase is an E. coli Pol I DNA polymerase. In some embodiments, the DNA polymerase is a Pol II family DNA polymerase. In some embodiments, the DNA polymerase is a Pyrococcusfuriosus (Pfu) Pol II DNA polymerase. In some embodiments, the DNA Polymerase is a Pol III family DNA polymerase. In some embodiments, the DNA Polymerase is a Pol IV family DNA polymerase. In some embodiments, the DNA polymerase is an E. coli Pol IV DNA polymerase. In some embodiments, the Pol I DNA polymerase is a DNA polymerase functional variant that lacks or has reduced 5′ to 3′ exonuclease activity.

Suitable thermostable pol I DNA polymerases can be isolated from a variety of thermophilic eubacteria, including Thermus species and Thermotoga maritima such as Thermus aquaticus (Taq), Thermus thermophilus (Tth) and Thermotoga maritima (Tma UlTma).

In some embodiments, a prime editor comprises an RNA-dependent DNA polymerase domain, for example, a reverse transcriptase (RT). A RT or an RT domain may be a wild type RT domain, a full-length RT domain, or may be a functional mutant, a functional variant, or a functional fragment thereof. An RT or an RT domain of a prime editor may comprise a wild-type RT, or may be engineered or evolved to contain specific amino acid substitutions, truncations, or variants. An engineered RT may comprise sequences or amino acid changes different from a naturally occurring RT. In some embodiments, the engineered RT may have improved reverse transcription activity over a naturally occurring RT or RT domain. In some embodiments, the engineered RT may have improved features over a naturally occurring RT, for example, improved thermostability, reverse transcription efficiency, or target fidelity. In some embodiments, a prime editor comprising the engineered RT has improved prime editing efficiency over a prime editor having a reference naturally occurring RT.

In some embodiments, a prime editor comprises a virus RT, for example, a retrovirus RT. Non-limiting examples of virus RT include Moloney murine leukemia virus (M-MLV, M-MLV RT, MMLVRT, or MMLV-RT); human T-cell leukemia virus type 1 (HTLV-1) RT; bovine leukemia virus (BLV) RT; Rous Sarcoma Virus (RSV) RT; human immunodeficiency virus (HIV) RT, M-MFV RT, Avian Sarcoma-Leukosis Virus (ASLV) RT, Rous Sarcoma Virus (RSV) RT, Avian Myeloblastosis Virus (AMV) RT, Avian Erythroblastosis Virus (AEV) Helper Virus MCAV RT, Avian Myelocytomatosis Virus MC29 Helper Virus MCAV RT, Avian Reticuloendotheliosis Virus (REV-T) Helper Virus REV-A RT, Avian Sarcoma Virus UR2 Helper Virus (UR2AV) RT, Avian Sarcoma Virus Y73 Helper Virus YAV RT, Rous Associated Virus (RAV) RT, and Myeloblastosis Associated Virus (MAV) RT, all of which may be suitably used in the methods and composition described herein.

In some embodiments, the prime editor comprises a wild type M-MLV RT, a functional mutant, a functional variant, or a functional fragment thereof. An exemplary sequence of a reference M-MLV RT is provided in SEQ ID NO: 590.

In some embodiments, the prime editor comprises a reference M-MLV RT, a functional mutant, a functional variant, or a functional fragment thereof. In some embodiments, the RT domain or a RT is a M-MLV RT (e.g., wild-type M-MLV RT, a functional mutant, a functional variant, or a functional fragment thereof). In some embodiments, the RT domain or a RT is a M-MLV RT (e.g., a reference M-MLV RT, a functional mutant, a functional variant, or a functional fragment thereof). In some embodiments, a M-MLV RT, e.g., reference M-MLV RT, comprises an amino acid sequence as set forth in SEQ ID NO: 590.

In some embodiments, a reference M-MLV RT is a wild-type M-MLV RT. An exemplary amino acid sequence of a reference M-MLV RT, wherein the reference M-MLV RT is a wild-type M-MLV RT is provided in SEQ ID NO: 589.

In some embodiments, the prime editor comprises a wild type M-MLV RT. An exemplary amino acid sequence of a wild type M-MLV RT is provided in SEQ ID NO: 589.

(SEQ ID NO: 589)
TLNIEDEHRLHETSKEPDVSLGSTWLSDFPQAWAETGGMGLAVRQAPLI
IPLKATSTPVSIKQYPMSQEARLGIKPHIQRLLDQGILVPCQSPWNTPL
LPVKKPGTNDYRPVQDLREVNKRVEDIHPTVPNPYNLLSGLPPSHQWYT
VLDLKDAFFCLRLHPTSQPLFAFEWRDPEMGISGQLTWTRLPQGFKNSP
TLFDEALHRDLADFRIQHPDLILLQYVDDLLLAATSELDCQQGTRALLQ
TLGNLGYRASAKKAQICQKQVKYLGYLLKEGQRWLTEARKETVMGQPTP
KTPRQLREFLGTAGFCRLWIPGFAEMAAPLYPLTKTGTLFNWGPDQQKA
YQEIKQALLTAPALGLPDLTKPFELFVDEKQGYAKGVLTQKLGPWRRPV
AYLSKKLDPVAAGWPPCLRMVAAIAVLTKDAGKLTMGQPLVILAPHAVE
ALVKQPPDRWLSNARMTHYQALLLDTDRVQFGPVVALNPATLLPLPEEG
LQHNCLDILAEAHGTRPDLTDQPLPDADHTWYTDGSSLLQEGQRKAGAA
VTTETEVIWAKALPAGTSAQRAELIALTQALKMAEGKKLNVYTDSRYAF
ATAHIHGEIYRRRGLLTSEGKEIKNKDEILALLKALFLPKRLSIIHCPG
HQKGHSAEARGNRMADQAARKAAITETPDTSTLLIENSSP.

In some embodiments, the prime editor comprises a reference M-MLV RT. An exemplary amino acid sequence of a reference M-MLV RT is provided in SEQ ID NO: 590.

(SEQ ID NO: 590)
TLNIEDEYRLHETSKEPDVSLGSTWLSDFPQAWAETGGMGLAVRQAPLI
IPLKATSTPVSIKQYPMSQEARLGIKPHIQRLLDQGILVPCQSPWNTPL
LPVKKPGTNDYRPVQDLREVNKRVEDIHPTVPNPYNLLSGLPPSHQWYT
VLDLKDAFFCLRLHPTSQPLFAFEWRDPEMGISGQLTWTRLPQGFKNSP
TLFDEALHRDLADFRIQHPDLILLQYVDDLLLAATSELDCQQGTRALLQ
TLGNLGYRASAKKAQICQKQVKYLGYLLKEGQRWLTEARKETVMGQPTP
KTPRQLREFLGTAGFCRLWIPGFAEMAAPLYPLTKTGTLFNWGPDQQKA
YQEIKQALLTAPALGLPDLTKPFELFVDEKQGYAKGVLTQKLGPWRRPV
AYLSKKLDPVAAGWPPCLRMVAAIAVLTKDAGKLTMGQPLVILAPHAVE
ALVKQPPDRWLSNARMTHYQALLLDTDRVQFGPVVALNPATLLPLPEEG
LQHNCLDILAEAHGTRPDLTDQPLPDADHTWYTDGSSLLQEGQRKAGAA
VTTETEVIWAKALPAGTSAQRAELIALTQALKMAEGKKLNVYTDSRYAF
ATAHIHGEIYRRRGLLTSEGKEIKNKDEILALLKALFLPKRLSIIHCPG
HQKGHSAEARGNRMADQAARKAAITETPDTSTLLIENSSP.

In some embodiments, the prime editor comprises a M-MLV RT comprising one or more of amino acid substitutions, for example, P51X, S67X, E69X, L139X, T197X, D200X, H204X, F209X, E302X, T306X, F309X, W313X, T330X, L345X, L435X, N454X, D524X, E562X, D583X, H594X, L603X, E607X, or D653X as compared to a reference M-MLV RT as set forth in SEQ ID NO: 590, where X is any amino acid other than the wild type amino acid. In some embodiments, the prime editor comprises a M-MLV RT comprising one or more of amino acid substitutions P51L, S67K, E69K, L139P, T197A, D200N, H204R, F209N, E302K, E302R, T306K, F309N, W313F, T330P, L345G, L435G, N454K, D524G, E562Q, D583N, H594Q, L603W, E607K, and/or D653N as compared to the reference M-MLV RT as set forth in SEQ ID NO: 590. In some embodiments, the prime editor comprises a M-MLV RT comprising one or more of amino acid substitutions, for example, D200N, T330P, L603W, T306K, or W313F as compared to a reference M-MLV RT as set forth in SEQ ID NO: 590. In some embodiments, the prime editor comprises a M-MLV RT comprising amino acid substitutions D200N, T330P, L603W, T306K, and W313F as compared to a reference M-MLV RT as set forth in SEQ ID NO: 590.

In some embodiments, a prime editor comprises a M-MLV RT comprising one or more of amino acid substitutions D200N, T330P, L603W, T306K, and W313F as compared to a wild type M-MLV RT as set forth in SEQ ID NO: 589. In some embodiments, the prime editor comprises a M-MLV RT that comprises an amino acid sequence that is at least 70%, at least 75%, at least 80%, at least 85%, at least 90%, at least 95%, at least 96%, at least 97%, at least 98%, at least 99%, at least 99.5%, or at least 99.9% identical to an amino acid sequence set forth in any one of SEQ ID NOs: 589, 590, or 591. In some embodiments, the prime editor comprises a M-MLV RT that comprises an amino acid sequence that is selected from the group consisting of SEQ ID NOs: 589, 590, and 591 or a variant or fragment thereof. In some embodiments, the prime editor comprises a M-MLV RT that comprises an amino acid sequence set forth in SEQ ID NO: 591.

(SEQ ID NO: 591)
TLNIEDEYRLHETSKEPDVSLGSTWLSDFPQAWAETGGMGLAVRQAPLI
IPLKATSTPVSIKQYPMSQEARLGIKPHIQRLLDQGILVPCQSPWNTPL
LPVKKPGTNDYRPVQDLREVNKRVEDIHPTVPNPYNLLSGLPPSHQWYT
VLDLKDAFFCLRLHPTSQPLFAFEWRDPEMGISGQLTWTRLPQGFKNSP
TLFNEALHRDLADFRIQHPDLILLQYVDDLLLAATSELDCQQGTRALLQ
TLGNLGYRASAKKAQICQKQVKYLGYLLKEGQRWLTEARKETVMGQPTP
KTPRQLREFLGKAGFCRLFIPGFAEMAAPLYPLTKPGTLFNWGPDQQKA
YQEIKQALLTAPALGLPDLTKPFELFVDEKQGYAKGVLTQKLGPWRRPV
AYLSKKLDPVAAGWPPCLRMVAAIAVLTKDAGKLTMGQPLVILAPHAVE
ALVKQPPDRWLSNARMTHYQALLLDTDRVQFGPVVALNPATLLPLPEEG
LQHNCLDILAEAHGTRPDLTDQPLPDADHTWYTDGSSLLQEGQRKAGAA
VTTETEVIWAKALPAGTSAQRAELIALTQALKMAEGKKLNVYTDSRYAF
ATAHIHGEIYRRRGWLTSEGKEIKNKDEILALLKALFLPKRLSIIHCPG
HQKGHSAEARGNRMADQAARKAAITETPDTSTLLIENSSP.

In some embodiments, an RT variant may be a functional fragment of a reference RT that has 1,2,3,4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or up to 100, or up to 200, or up to 300, or up to 400, or up to 500 or more amino acid changes compared to a wild type RT, e.g., SEQ ID NO: 589. In some embodiments, the RT variant comprises a fragment of a wild type RT, e.g., a wild type RT corresponding to SEQ ID NO: 589, such that the fragment is about 70% identical, about 80% identical, about 90% identical, about 95% identical, about 96% identical, about 97% identical, about 98% identical, about 99% identical, about 99.5% identical, or about 99.9% identical to the corresponding fragment of the wild type RT, e.g., a wild type RT corresponding to SEQ ID NO: 589. In some embodiments, the fragment is 30%, 35%, 40%, 45%, 50%, 55%, 60%, 65%, 70%, 75%, 80%, 85%, 90%, 95%, 96%, 97%, 98%, 99%, or 99.5% of the amino acid length of a corresponding wild type RT (e.g., M-MLV reverse transcriptase) (e.g., SEQ ID NO: 589).In some embodiments, an RT variant may be a functional fragment of a reference RT that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, orup to 100, or up to 200, or up to 300, or up to 400, or up to 500 or more amino acid changes compared to a reference RT, e.g., SEQ ID NO: 590. In some embodiments, the RT variant comprises a fragment of a reference RT, such that the fragment is about 70% identical, about 80% identical, about 90% identical, about 95% identical, about 96% identical, about 97% identical, about 98% identical, about 99% identical, about 99.5% identical, or about 99.9% identical to the corresponding fragment of a reference RT (e.g., SEQ ID NO: 590). In some embodiments, the fragment is 30%, 35%, 40%, 45%, 50%, 55%, 60%, 65%, 70%, 75%, 80%, 85%, 90%, 95% identical, 96%, 97%, 98%, 99%, or 99.5% of the amino acid length of a reference RT (e.g., a M-MLV RT) (e.g., SEQ ID NO: 590).

In some embodiments, the RT functional fragment is at least 100 amino acids in length. In some embodiments, the fragment is at least 100, 150, 200, 250, 300, 350, 400, 450, 500, 550, or up to 600 or more amino acids in length.

In still other embodiments, the functional RT variant is truncated at the N-terminus or the C-terminus, or both, by a certain number of amino acids which results in a truncated variant which still retains sufficient DNA polymerase function. In some embodiments, the RT.truncated variant has a truncation of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, 40, 50, 60, 70, 80, 90,100,110, 120, 130, 140, 150, 160, 170, 180, 190, 200, 210, 220, 230, 240, or 250 amino acids at the N-terminal end compared to a reference RT, e.g., a wild type RT. In other embodiments, the RT truncated variant has a truncation of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, 40, 50, 60, 70, 80, 90,100,110, 120, 130, 140, 150, 160, 170, 180, 190, 200, 210, 220, 230, 240, or 250 amino acids at the C-terminal end compared to a reference RT, e.g., a wild type RT. In some embodiments, the reference RT is a wild type M-MLV RT. In still other embodiments, the RT truncated variant has a truncation at the N-terminal and the C-terminal end compared to a reference RT, e.g., a wild type RT. In some embodiments, the N-terminal truncation and the C-terminal truncation are of the same length. In some embodiments, the N-terminal truncation and the C-terminal truncation are of different lengths. In some embodiments, the functional RT variant, e.g., a functional M-MLV RT variant, is truncated at the C-terminus to abolish or reduce RNAaseH activity and still retain DNA polymerase activity.

For example, the prime editors disclosed herein may include a functional variant of a wild type M-MLV reverse transcriptase. In some embodiments, the prime editor comprises a functional variant of a wild type M-MLV RT, wherein the functional variant of M-MLV RT is truncated after amino acid position 502 compared to a wild type M-MLV RT as set forth in SEQ ID NO: 589. In some embodiments, the prime editor comprises a functional variant of a reference M-MLV RT, wherein the functional variant of M-MLV RT is truncated after amino acid position 502 compared to a reference type M-MLV RT as set forth in SEQ ID NO: 590. In some embodiments, the functional variant of M-MLV RT further comprises a D200X, T306X, W313X, and/or T330X amino acid substitution compared to a wild type M-MLV RT as set forth in SEQ ID NO: 589, wherein X is any amino acid other than the original amino acid. In some embodiments, the functional variant of M-MLV RT further comprises a D200N, T306K, W313F, and/or T330P amino acid substitution compared to a wild type M-MLV RT as set forth in SEQ ID NO: 589, wherein X is any amino acid other than the original amino acid. In some embodiments, the nucleotide polymerase domain is a polynucleotide polymerase domain.

In some embodiments, a prime editing composition or a prime editing system disclosed herein comprises a polynucleotide (e.g., a DNA, a RNA, e.g., a mRNA) that encodes a M-MLV RT. In some embodiments, the polynucleotide encodes a M-MLV RT polypeptide that comprises an amino acid sequence that comprises at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.9% or 100% identity to an amino acid sequence set forth in any one of SEQ ID NOs: 589, 590, or 591. In some embodiments, the polynucleotide encodes a M-MLV RT that comprises an amino acid sequence that is selected from the group consisting of SEQ ID NOs: 589, 590, and 591. In some embodiments, the polynucleotide encodes a M-MLV RT that comprises an amino acid sequence that is set forth in SEQ ID NO: 591.

In some embodiments, a prime editor comprises a eukaryotic RT, for example, a yeast, drosophila, rodent, or primate RT. In some embodiments, the prime editor comprises a Group II intron RT, for example, a. Geobacillus stearothermophilus Group II Intron (GsI-IIC) RT or a Eubacterium rectale group II intron (Eu.re.I2) RT. In some embodiments, the prime editor comprises a retron RT. In some embodiments, a prime editor comprises a eukaryotic RT, for example, a yeast, drosophila, rodent, or primate RT. In some embodiments, the prime editor comprises a Group II intron RT, for example, a. Geobacillus stearothermophilus Group II Intron (GsI-IIC) RT or a Eubacterium rectale group II intron (Eu.re.I2) RT. In some embodiments, the prime editor comprises a retron RT.

### Programmable DNA Binding Domain

In some embodiments, the DNA-binding domain of a prime editor is a programmable DNA binding domain. In some embodiments, a prime editor comprises a DNA binding domain that comprises an amino acid sequence that is at least 50%, at least 60%, at least 70%, at least 75%, at least 80%, 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of the sequences set forth in SEQ ID NOs: 592-619.

In some embodiments, the DNA binding domain comprises an amino acid sequence that has no more than 1, 2, 3, 4, 5, 6, 7, 8, 9, 10, 11, 12, 13, 14, 15, 16, 17, 18, 19, or 20 differences e.g., mutations e.g., deletions, substitutions and/or insertions compared to any one of the amino acid sequences set forth in SEQ ID NOs: 592-619.

In some embodiments, the DNA binding domain of a prime editor is a programmable DNA binding domain. A programmable DNA binding domain refers to a protein domain that is designed to bind a specific nucleic acid sequence, e.g., a target DNA or a target RNA. In some embodiments, the DNA-binding domain is a polynucleotide programmable DNA-binding domain that can associate with a guide polynucleotide (e.g., a PEgRNA) that guides the DNA-binding domain to a specific DNA sequence, e.g., a search target sequence in a target gene.

In some embodiments, the DNA-binding domain comprises a Clustered Regularly Interspaced Short Palindromic Repeats (CRISPR) Associated (Cas) protein. A Cas protein may comprise any Cas protein described herein or a functional fragment or functional variant thereof. In some embodiments, a DNA-binding domain may also comprise a zinc-finger protein domain. In other cases, a DNA-binding domain comprises a transcription activator-like effector domain (TALE). In some embodiments, the DNA-binding domain comprises a DNA nuclease. For example, the DNA-binding domain of a prime editor may comprise an RNA-guided DNA endonuclease, e.g., a Cas protein. In some embodiments, the DNA-binding domain comprises a zinc finger nuclease (ZFN) or a transcription activator like effector domain nuclease (TALEN), where one or more zinc finger motifs or TALE motifs are associated with one or more nucleases, e.g., a Fok I nuclease domain.

In some embodiments, the DNA-binding domain comprises a nuclease activity. In some embodiments, the DNA-binding domain of a prime editor comprises an endonuclease domain having single strand DNA cleavage activity. For example, the endonuclease domain may comprise a FokI nuclease domain. In some embodiments, the DNA-binding domain of a prime editor comprises a nuclease having full nuclease activity. In some embodiments, the DNA-binding domain of a prime editor comprises a nuclease having modified or reduced nuclease activity as compared to a wild type endonuclease domain. For example, the endonuclease domain may comprise one or more amino acid substitutions as compared to a wild type endonuclease domain. In some embodiments, the DNA-binding domain of a prime editor comprises a nickase activity. In some embodiments, the DNA-binding domain of a prime editor comprises a Cas protein domain that is a nickase. In some embodiments, compared to a wild type Cas protein, the Cas nickase comprises one or more amino acid substitutions in a nuclease domain that reduces or abolishes its double strand nuclease activity but retains DNA binding activity. In some embodiments, the Cas nickase comprises an amino acid substitution in a HNH domain. In some embodiments, the Cas nickase comprises an amino acid substitution in a RuvC domain.

In some embodiments, the DNA-binding domain comprises a CRISPR associated protein (Cas protein) domain. A Cas protein may be a Class 1 or a Class 2 Cas protein. A Cas protein can be a type I, type II, type III, type IV, type V Cas protein, or a type VI Cas protein. Non-limiting examples of Cas proteins include Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9 (e.g., Csn1 or Csx12), Cas10, Cas1Od, Cas12a/Cpf1, Cas12b/C2c1, Cas12c/C2c3, Cas12d/CasY, Cas12e/CasX, Cas12g, Cas12h, Cas12i, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx1O, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, Type II Cas effector proteins, Type V Cas effector proteins, Type VI Cas effector proteins, CARF, DinG, Cpf1, Cas12b/C2c1, Cas12c/C2c3, Cas12b/C2c1, Cas12c/C2c3, SpCas9(K855A), eSpCas9(1.1), SpCas9-HF1, hyper accurate Cas9 variant (HypaCas9), Cas ( ), and homologues, modified or engineered variants, mutants, and/or functional fragments thereof. A Cas protein can be a chimeric Cas protein that is fused to other proteins or polypeptides. A Cas protein can be a chimera of various Cas proteins, for example, comprising domains of Cas proteins from different organisms. A Cas protein, e.g., Cas9, can be from any suitable organism. A Cas protein, e.g., Cas9, can be derived from any suitable organism. In some embodiments, the organism is Streptococcus pyogenes (S. pyogenes). In some embodiments, the organism is Staphylococcus aureus (S. aureus). In some embodiments, the organism is Streptococcus thermophilus (S. thermophilus). In some embodiments, the organism is Staphylococcus lugdunensis (S. lugdunensis). Non-limiting examples of suitable organisms include Streptococcus pyogenes, Streptococcus thermophilus, Streptococcus sp., Staphylococcus aureus, Nocardiopsis dassonvillei, Streptomyces pristinae spiralis, Streptomyces viridochromo genes, Streptomyces viridochromogenes, Streptosporangium roseum, Streptosporangium roseum, AlicyclobacHlus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Pseudomonas aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Leptotrichia shahii, and Francisella novicida.

A Cas protein, e.g., Cas9, can be from any suitable organism. In some aspects, the organism is Streptococcus pyogenes (S. pyogenes). In some aspects, the organism is Staphylococcus aureus (S. aureus). In some aspects, the organism is Streptococcus thermophilus (S. thermophilus). In some embodiments, the organism is Staphylococcus lugdunensis (S. lugdunensis).

In some embodiments, a Cas protein can be derived from one or more bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractor salsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinella succinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida.

In some embodiments, a Cas protein, e.g., Cas9, can be a wild type or a modified form of a Cas protein. In some embodiments, a Cas protein, e.g., Cas9, can be a nuclease active variant, nuclease inactive variant, a nickase, or a functional variant or a functional fragment of a wild type Cas protein. In some embodiments, a Cas protein, e.g., Cas9, can be a wild type or a modified form of a Cas protein. In some embodiments, a Cas protein, e.g., Cas9, can be a nuclease active variant, nuclease inactive variant, a nickase, or a functional variant or functional fragment of a wild type Cas protein. In some embodiments, a Cas protein, e.g., Cas9, can comprise an amino acid change such as a deletion, insertion, substitution, fusion, chimera, or any combination thereof relative to a corresponding wild-type version of the Cas protein. In some embodiments, a Cas protein can be a polypeptide with at least about 50%, 60%, 70%, 80%, 90%, 91%, 92%, 93%, 94%, 95%, 96%, 97%, 98%, 99%, or 100% sequence identity or sequence similarity to a wild type exemplary Cas protein.

A Cas protein, e.g., Cas9, may comprise one or more domains. Non-limiting examples of Cas domains include, guide nucleic acid recognition and/or binding domain, nuclease domains (e.g., DNase or RNase domains, RuvC, HNH), DNA binding domain, RNA binding domain, helicase domains, protein-protein interaction domains, and dimerization domains. In various embodiments, a Cas protein comprises a guide nucleic acid recognition and/or binding domain can interact with a guide nucleic acid, and one or more nuclease domains that comprise catalytic activity for nucleic acid cleavage.

In some embodiments, a Cas protein, e.g., Cas9, comprises one or more nuclease domains. A Cas protein can comprise an amino acid sequence having at least about 50%, 60%, 70%, 80%, 90%, 91%, 92%, 93%, 94%, 95%, 96%, 97%, 98%, 99%, or 100% sequence identity to a nuclease domain (e.g., RuvC domain, HNH domain) of a wild-type Cas protein. In some embodiments, a Cas protein comprises a single nuclease domain. For example, a Cpf1 may comprise a RuvC domain but lacks HNH domain. In some embodiments, a Cas protein comprises two nuclease domains, e.g., a Cas9 protein can comprise an HNH nuclease domain and a RuvC nuclease domain.

In some embodiments, a prime editor comprises a Cas protein, e.g., Cas9, wherein all nuclease domains of the Cas protein are active. In some embodiments, a prime editor comprises a Cas protein having one or more inactive nuclease domains. One or a plurality of the nuclease domains (e.g., RuvC, HNH) of a Cas protein can be deleted or mutated so that they are no longer functional or comprise reduced nuclease activity. In some embodiments, a Cas protein, e.g., Cas9, comprising mutations in a nuclease domain has reduced (e.g., nickase) or abolished nuclease activity while maintaining its ability to target a nucleic acid locus at a search target sequence when complexed with a guide nucleic acid, e.g., a PEgRNA.

In some embodiments, a prime editor comprises a Cas nickase that can bind to the target gene in a sequence-specific manner and generate a single-strand break at a protospacer within double-stranded DNA in the target gene, but not a double-strand break. For example, the Cas nickase can cleave the edit strand or the non-edit strand of the target gene, but may not cleave both. In some embodiments, a prime editor comprises a Cas nickase comprising two nuclease domains (e.g., Cas9), with one of the two nuclease domains modified to lack catalytic activity or deleted. In some embodiments, the Cas nickase of a prime editor comprises a nuclease inactive RuvC domain and a nuclease active HNH domain. In some embodiments, the Cas nickase of a prime editor comprises a nuclease inactive HNH domain and a nuclease active RuvC domain. In some embodiments, a prime editor comprises a Cas9 nickase having an amino acid substitution in the RuvC domain e.g., an amino acid substitution that reduces or abolishes nuclease activity of the RuvC domain. In some embodiments, the Cas9 nickase comprises a D1OX amino acid substitution compared to a wild type S. pyogenes Cas9, wherein X is any amino acid other than D. In some embodiments, a prime editor comprises a Cas9 nickase having an amino acid substitution in the HNH domain e.g., an amino acid substitution that reduces or abolishes nuclease activity of the HNH domain. In some embodiments, the Cas9 nickase comprises a H840X amino acid substitution compared to a wild type S. pyogenes Cas9, wherein X is any amino acid other than H.

In some embodiments, a prime editor comprises a Cas protein that can bind to the target gene in a sequence-specific manner but lacks or has abolished nuclease activity and may not cleave either strand of a double stranded DNA in a target gene. Abolished activity or lacking activity can refer to an enzymatic activity less than 1%, less than 2%, less than 3%, less than 4%, less than 5%, less than 6%, less than 7%, less than 8%, less than 9%, or less than 10% activity compared to a wild-type exemplary activity (e.g., wild-type Cas9 nuclease activity). In some embodiments, a Cas protein of a prime editor completely lacks nuclease activity. A nuclease, e.g., Cas9, that lacks nuclease activity may be referred to as nuclease inactive or “nuclease dead” (abbreviated by “d”). A nuclease dead Cas protein (e.g., dCas, dCas9) can bind to a target polynucleotide but may not cleave the target polynucleotide. In some aspects, a dead Cas protein is a dead Cas9 protein. In some embodiments, a prime editor comprises a nuclease dead Cas protein wherein all of the nuclease domains (e.g., both RuvC and HNH nuclease domains in a Cas9 protein; RuvC nuclease domain in a Cpf1 protein) are mutated to lack catalytic activity, or are deleted.

A Cas protein can be modified. A Cas protein, e.g., Cas9, can be modified to increase or decrease nucleic acid binding affinity, nucleic acid binding specificity, and/or enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of the Cas protein.

A Cas protein can be a fusion protein. For example, a Cas protein can be fused to a cleavage domain, an epigenetic modification domain, a transcriptional regulation domain, or a polymerase domain. A Cas protein can also be fused to a heterologous polypeptide providing increased or decreased stability. The fused domain or heterologous polypeptide can be located at the N-terminus, the C-terminus, or internally within the Cas protein.

In some embodiments, the Cas protein of a prime editor is a Class 2 Cas protein. In some embodiments, the Cas protein is a type II Cas protein. In some embodiments, the Cas protein is a Cas9 protein, a modified version of a Cas9 protein, a Cas9 protein homolog, mutant, variant, or a functional fragment thereof. As used herein, a Cas9, Cas9 protein, Cas9 polypeptide or a Cas9 nuclease refers to an RNA guided nuclease comprising one or more Cas9 nuclease domains and a Cas9 gRNA binding domain having the ability to bind a guide polynucleotide, e.g., a PEgRNA. A Cas9 protein may refer to a wild type Cas9 protein from any organism or a homolog, ortholog, or paralog from any organisms; any functional mutants or functional variants thereof, or any functional fragments or domains thereof. In some embodiments, a prime editor comprises a full-length Cas9 protein. In some embodiments, the Cas9 protein can generally comprises at least about 50%, 60%, 70%, 80%, 90%, 100% sequence identity to a wild type reference Cas9 protein (e.g., Cas9 from S. pyogenes). In some embodiments, the Cas9 comprises an amino acid change such as a deletion, insertion, substitution, fusion, chimera, or any combination thereof as compared to a wild type reference Cas9 protein.

In some embodiments, a Cas9 protein may comprise a Cas9 protein from Streptococcus pyogenes (Sp), Staphylococcus aureus (Sa), Streptococcus canis (Sc), Streptococcus thermophilus (St), Staphylococcus lugdunensis (Slu), Neisseria meningitidis (Nm), Campylobacter jejuni (Cj), Francisella novicida (Fn), or Treponema denticola (Td), or any Cas9 homolog or ortholog from an organism known in the art.

In some embodiments, a Cas9 polypeptide is a SpCas9 polypeptide, e.g., comprising an amino acid sequence as set forth in NCBI Accession No. WP_038431314 or a fragment or variant thereof. In some embodiments, a Cas9 polypeptide is a SaCas9 polypeptide, e.g., comprising an amino acid sequence as set forth in Uniprot Accession No. J7RUA5 or a fragment or variant thereof. In some embodiments, a Cas9 polypeptide is a ScCas9 polypeptide, e.g., comprising an amino acid sequence as set forth in Uniprot Accession No. AOA3P5YA78 or a fragment or variant thereof. In some embodiments, a Cas9 polypeptide is a StCas9 polypeptide, e.g., comprising an amino acid sequence as set forth in NCBI Accession No. WP_007896501.1 or a fragment or variant thereof. In some embodiments, a Cas9 polypeptide is a SluCas9 polypeptide, e.g., comprising an amino acid sequence as set forth in any of NCBI Accession No. WP_230580236.1 or WP_250638315.1 or WP_242234150.1, WP_241435384.1, WP_002460848.1, KAK58371.1, or a fragment or variant thereof. In some embodiments, a Cas9 polypeptide is a NmCas9 polypeptide, e.g., comprising an amino acid sequence as set forth in any of NCBI Accession No. WP_002238326.1 or WP_061704949.1 or a fragment or variant thereof. In some embodiments, a Cas9 polypeptide is a CjCas9 polypeptide, e.g., comprising an amino acid sequence as set forth in any of NCBI Accession No. WP_100612036.1, WP 116882154.1, WP 116560509.1, WP_116484194.1, WP_116479303.1, WP_115794652.1, WP_100624872.1, or a fragment or variant thereof. In some embodiments, a Cas9 polypeptide is a FnCas9 polypeptide, e.g., comprising the amino acid sequence as set forth in Uniprot Accession No. AOQ5Y3 or a fragment or variant thereof. In some embodiments, a Cas9 polypeptide is a TdCas9 polypeptide, e.g., comprising the amino acid sequence as set forth in NCBI Accession No. WP_147625065.1 or a fragment or variant thereof. In some embodiments, a Cas9 polypeptide is a chimera comprising domains from two or more of the organisms described herein or those known in the art. In some embodiments, a Cas9 polypeptide is a Cas9 polypeptide from Streptococcus macacae, e.g., comprising the amino acid sequence as set forth in NCBI Accession No. WP_003079701.1 or a fragment or variant thereof. In some embodiments, a Cas9 polypeptide is a Cas9 polypeptide generated by replacing a PAM interaction domain of a SpCas9 with that of a Streptococcus macacae Cas9 (Spy-mac Cas9).

In some embodiments, a Cas9 polypeptide is a SpCas9 polypeptide. In some embodiments, a Cas9 polypeptide is a SaCas9 polypeptide. In some embodiments, a Cas9 polypeptide is a ScCas9 polypeptide. In some embodiments, a Cas9 polypeptide is a StCas9 polypeptide. In some embodiments, a Cas9 polypeptide is a SluCas9 polypeptide. In some embodiments, a Cas9 polypeptide is a NmCas9 polypeptide. In some embodiments, a Cas9 polypeptide is a CjCas9 polypeptide. In some embodiments, a Cas9 polypeptide is a FnCas9 polypeptide. In some embodiments, a Cas9 polypeptide is a TdCas9 polypeptide. In some embodiments, a Cas9 polypeptide is a chimera comprising domains from two or more of the organisms described herein or known in the art. In some embodiments, a Cas9 polypeptide is a Cas9 polypeptide from Streptococcus macacae. In some embodiments, a Cas9 polypeptide is a Cas9 polypeptide generated by replacing a PAM interaction domain of a SpCas9 with that of a Streptococcus macacae Cas9 (Spy-mac Cas9).

In some embodiments, a Cas9 protein comprises an amino acid sequence that is at least 50%, at least 60%, at least 70%, at least 75%, at least 80%, 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of the sequences set forth in SEQ ID NOs: 592-619.

In some embodiments, a Cas9 protein comprises an amino acid sequence that is at least 50%, at least 60%, at least 70%, at least 75%, at least 80%, 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of the sequences set forth in Table 7.

In some embodiments, a Cas9 protein is a Cas9 nickase that comprises an amino acid sequence that is at least 50%, at least 60%, at least 70%, at least 75%, at least 80%, 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of the sequences set forth in SEQ ID NOs: 593, 594, 596, 597, 599, 600, 602, 603, 605, 606, 608, 609, 611, 612, 614, 615, 617, 618, or 619.

In some embodiments, a Cas9 protein is a Cas9 nickase that comprises an amino acid sequence that is at least 50%, at least 60%, at least 70%, at least 75%, at least 80%, 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of the nickase sequences set forth in Table 7.

In some embodiments, a Cas9 protein comprises an amino acid sequence that is selected from the group consisting of SEQ ID NOs: 592-619.

In some embodiments, a Cas9 protein comprises an amino acid sequence that is selected from the group consisting of the sequences set forth in Table 7.

In some embodiments, a prime editor comprises a Cas9 protein that comprises an amino acid sequence that lacks a N-terminus methionine relative to an amino acid sequence set forth in any one of SEQ ID NOs: 592, 593, 595, 596, 598, 599, 601, 602, 604, 605, 607, 608, 610, 611, 613, 614, 616, or 617.

In some embodiments, the prime editing compositions or prime editing systems disclosed herein comprises a polynucleotide (e.g., a DNA, or an RNA, e.g., an mRNA) that encodes a Cas9 protein that comprises an amino acid sequence that is at least 50%, at least 60%, at least 70%, at least 75%, at least 80%, 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of the sequences set forth in SEQ ID NOs: 592-619, set forth in Table 7.

In some embodiments, a Cas9 protein comprises a Cas9 protein from Streptococcus pyogenes (Sp), e.g., as according to NC_002737.2:854751-858857 or the protein encoded by UniProt Q99ZW2, e.g., as according to SEQ ID NO: 592. In some embodiments, a prime editor comprises a Cas9 protein as according to any one of the sequences set forth in SEQ ID NOs: 592-619 or a variant thereof. In some embodiments, the Cas9 protein is a SpCas9. In some embodiments, a SpCas9 can be a wild type SpCas9, a SpCas9 variant, or a nickase SpCas9. In some embodiments, the SpCas9 lacks the N-terminus methionine relative to a corresponding SpCas9 (e.g., a wild type SpCas9, a SpCas9 variant or a nickase SpCas9). In some embodiments, a prime editor comprises a Cas9 protein, having an amino acid sequence as according to SEQ ID NO: 594, not including the N-terminus methionine. In some embodiments, a prime editor comprises a Cas9 protein, having an amino acid sequence as according to SEQ ID NO: 619, not including the N-terminus methionine. In some embodiments, a wild type SpCas9 comprises an amino acid sequence set forth in SEQ ID NO: 592. In some embodiments, a prime editor comprises a Cas9 protein comprising one or more mutations (e.g., amino acid substitutions, insertions and/or deletions) relative to a corresponding wild type Cas9 protein (e.g., a wild type SpCas9). Exemplary Streptococcus pyogenes Cas9 (SpCas9) amino acid sequence useful in the prime editors disclosed herein are provided below in SEQ ID NOs: 592, 593, 594, 601-609, and 616-619.

In some embodiments, a prime editor comprises a Cas9 protein from Staphylococcus lugdunensis (SluCas9) e.g., as according to or derived from any one of the SEQ ID NOs: 595, 596, 597, or a variant thereof. In some embodiments, the Cas9 protein is a SluCas9. In some embodiments, a SluCas9 can be a wild type SluCas9, a SluCas9 variant, or a nickase SluCas9. In some embodiments, the SluCas9 lacks the N-terminus methionine relative to a corresponding SluCas9 (e.g., a wild type SluCas9, a SluCas9 variant or a nickase SluCas9). In some embodiments, a prime editor comprises a Cas9 protein, having an amino acid sequence as according to SEQ ID NO: 597, not including the N-terminus methionine. In some embodiments, a wild type SluCas9 comprises an amino acid sequence set forth in SEQ ID NO: 595. In some embodiments, a prime editor comprises a Cas9 protein comprising one or more mutations (e.g., amino acid substitutions, insertions and/or deletions) relative to a corresponding wild type Cas9 protein (e.g., a wild type SluCas9). In some embodiments, the Cas9 protein comprising one or mutations relative to a wild type Cas9 protein comprises an amino acid sequence set forth in SEQ ID NO: 596 or 597.

In some embodiments, a prime editor comprises a Cas9 protein from Staphylococcus aureus (SaCas9) e.g., as according to or derived from any of the SEQ ID NOS: 598, 599, 600, or a variant thereof. In some embodiments, the Cas9 protein is a SaCas9. In some embodiments, a SaCas9 can be a wild type SaCas9, a SaCas9 variant, or a nickase SaCas9. In some embodiments, the SaCas9 lacks the N-terminus methionine relative to a corresponding SaCas9 (e.g., a wild type SaCas9, a SaCas9 variant or a nickase SaCas9). In some embodiments, a prime editor comprises a Cas9 protein, having an amino acid sequence as according to SEQ ID NO: 600, not including the N-terminus methionine. In some embodiments, a wild type SaCas9 comprises an amino acid sequence set forth in SEQ ID NO: 598. In some embodiments, a prime editor comprises a Cas9 protein comprising one or more mutations (e.g., amino acid substitutions, insertions and/or deletions relative to a corresponding wild type Cas9 protein (e.g., a wild type SaCas9). In some embodiments, the Cas9 protein comprising one or more mutations relative to a wild type Cas9 protein comprises an amino acid sequence set forth in SEQ ID NO: 599 or 600. Exemplary Staphylococcus aureus Cas9 (SaCas9) amino acid sequence useful in the prime editors disclosed herein are provided below in SEQ ID NOs: 598, 599, and 600.

In some embodiments, a prime editor comprises a Cas protein, e.g., a Cas9 variant, comprising modifications that allow altered PAM recognition. Exemplary Cas9 protein amino acid sequence (e.g., Cas9 variant with altered PAM recognition specificities) that are useful in the Prime editors of the disclosure are provided herein.

In some embodiments, a prime editor comprises a Cas9 protein as according to any one of the sequences set forth in SEQ ID NOs: 592-619, or a variant thereof. In some embodiments, the Cas9 protein is a Cas9 variant, for example, a SpCas9 variant (e.g., SpCas9-NG, SpCas9-NGA, SpRY, or SpG).

In some embodiments, the Cas9 protein lacks the N-terminus methionine relative to a corresponding Cas9 protein. In some embodiments, a prime editor comprises a Cas9 protein comprising one or more mutations (e.g., amino acid substitutions, insertions and/or deletions) relative to a corresponding Cas9 protein (e.g., a Cas9 protein set forth in any one of SEQ ID NOs: 592-619).

In some embodiments, a Cas9 protein is a chimeric Cas9, e.g., modified Cas9, e.g., synthetic RNA-guided nucleases (sRGNs), e.g., modified by DNA family shuffling, e.g., sRGN3.1, sRGN3.3. In some embodiments, the DNA family shuffling comprises, fragmentation and reassembly of parental Cas9 genes, e.g., one or more of Cas9s from Staphylococcus hyicus (Shy), Staphylococcus lugdunensis (Slu), Staphylococcus microti (Smi), and Staphylococcus pasteuri (Spa). In some embodiments, a modified Cas9 shows increased editing efficiency and/or specificity relative to a Cas9 that is not modified. In some embodiments, a modified Cas9, e.g., a sRGN shows at least 10%, at least 20%, at least 30%, at least 40%, at least 50%, at least 60%, at least 70%, at least 80%, at least 90%, at least 100%, at least 150%, at least 200%, at least 300%, at least 400%, at least 500%, at least 600%, at least 700%, at least 800%, at least 900%, or at least 1000% increase in editing efficiency compared to a Cas9 that is not modified. In some embodiments, a Cas9, e.g., a sRGN shows at least 10%, at least 20%, at least 30%, at least 40%, at least 50%, at least 60%, at least 70%, at least 80%, at least 90%, at least 100%, at least 150%, at least 200%, at least 300%, at least 400%, at least 500%, at least 600%, at least 700%, at least 800%, at least 900%, or at least 1000% increase in specificity compared to a Cas9 that is not modified. In some embodiments, a Cas9, e.g., a sRGN shows at least 10%, at least 20%, at least 30%, at least 40%, at least 50%, at least 60%, at least 70%, at least 80%, at least 90%, at least 100%, at least 150%, at least 200%, at least 300%, at least 400%, at least 500%, at least 600%, at least 700%, at least 800%, at least 900%, or at least 1000% increase in cleavage activity compared to a Cas9 that is not modified. In some embodiments, a Cas9, e.g., a sRGN shows ability to cleave a 5′-NNGG-3′ PAM-containing target. In some embodiments, a prime editor comprises a Cas9 protein (e.g., a chimeric Cas9), e.g., as according any one of the sequences set forth in SEQ ID NOs: 610-615, or a variant thereof. Exemplary amino acid sequences of Cas9 protein (e.g., sRGN) useful in the prime editors disclosed herein are provided below in SEQ ID NOs: 610-615.

Exemplary Cas protein sequences are provided in Table 7.

TABLE 7
Exemplary Cas protein sequences

| SEQ | | |
|---|---|---|
| ID | Sequence | |
| NO: | description | Amino acid sequence |
| 592 | wild type | MDKKYSIGLDIGTNSVGWAVITDEYKVPSKKFKVLGNTDRHSIKKNLIGALLFDSGE |
| | Streptococcus | TAEATRLKRTARRRYTRRKNRICYLQEIFSNEMAKVDDSFFHRLEESFLVEEDKKHE |
| | Pyogenes Cas9 | RHPIFGNIVDEVAYHEKYPTIYHLRKKLVDSTDKADLRLIYLALAHMIKFRGHFLIEG |
| | (SpCas9) | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDHIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFDSPTV |
| | | AYSVLVVAKVEKGKSKKLKSVKELLGITIMERSSFEKNPIDFLEAKGYKEVKKDLIIK |
| | | LPKYSLFELENGRKRMLASAGELQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKRYTSTKEVLDATLIHQSITGLYETRIDLSQLGGD |
| 593 | SpCas9 H840A | MDKKYSIGLDIGTNSVGWAVITDEYKVPSKKFKVLGNTDRHSIKKNLIGALLFDSGE |
| | nickase | TAEATRLKRTARRRYTRRKNRICYLQEIFSNEMAKVDDSFFHRLEESFLVEEDKKHE |
| | | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFDSPTV |
| | | AYSVLVVAKVEKGKSKKLKSVKELLGITIMERSSFEKNPIDFLEAKGYKEVKKDLIIK |
| | | LPKYSLFELENGRKRMLASAGELQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKRYTSTKEVLDATLIHQSITGLYETRIDLSQLGGD |
| 594 | Met(−) SpCas9 | DKKYSIGLDIGTNSVGWAVITDEYKVPSKKFKVLGNTDRHSIKKNLIGALLFDSGET |
| | H840A nickase | AEATRLKRTARRRYTRRKNRICYLQEIFSNEMAKVDDSFFHRLEESFLVEEDKKHER |
| | | HPIFGNIVDEVAYHEKYPTIYHLRKKLVDSTDKADLRLIYLALAHMIKFRGHFLIEGD |
| | | LNPDNSDVDKLFIQLVQTYNQLFEENPINASGVDAKAILSARLSKSRRLENLIAQLPG |
| | | EKKNGLFGNLIALSLGLTPNFKSNFDLAEDAKLQLSKDTYDDDLDNLLAQIGDQYA |
| | | DLFLAAKNLSDAILLSDILRVNTEITKAPLSASMIKRYDEHHQDLTLLKALVRQQLPE |
| | | KYKEIFFDQSKNGYAGYIDGGASQEEFYKFIKPILEKMDGTEELLVKLNREDLLRKQ |
| | | RTFDNGSIPHQIHLGELHAILRRQEDFYPFLKDNREKIEKILTFRIPYYVGPLARGNSR |
| | | FAWMTRKSEETITPWNFEEVVDKGASAQSFIERMTNFDKNLPNEKVLPKHSLLYEY |
| | | FTVYNELTKVKYVTEGMRKPAFLSGEQKKAIVDLLFKTNRKVTVKQLKEDYFKKIE |
| | | CFDSVEISGVEDRFNASLGTYHDLLKIIKDKDFLDNEENEDILEDIVLTLTLFEDREMI |
| | | EERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFDSPTV |
| | | AYSVLVVAKVEKGKSKKLKSVKELLGITIMERSSFEKNPIDFLEAKGYKEVKKDLIIK |
| | | LPKYSLFELENGRKRMLASAGELQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKRYTSTKEVLDATLIHQSITGLYETRIDLSQLGGD |
| 595 | wild type | MNQKFILGLDIGITSVGYGLIDYETKNIIDAGVRLFPEANVENNEGRRSKRGSRRLKR |
| | Staphylococcus | RRIHRLERVKKLLEDYNLLDQSQIPQSTNPYAIRVKGLSEALSKDELVIALLHIAKRR |
| | lugdunensis | GIHKIDVIDSNDDVGNELSTKEQLNKNSKLLKDKFVCQIQLERMNEGQVRGEKNRF |
| | (Slu) Cas9 | KTADIIKEIIQLLNVQKNFHQLDENFINKYIELVEMRREYFEGPGKGSPYGWEGDPK |
| | | AWYETLMGHCTYFPDELRSVKYAYSADLFNALNDLNNLVIQRDGLSKLEYHEKYH |
| | | IIENVFKQKKKPTLKQIANEINVNPEDIKGYRITKSGKPQFTEFKLYHDLKSVLFDQSI |
| | | LENEDVLDQIAEILTIYQDKDSIKSKLTELDILLNEEDKENIAQLTGYTGTHRLSLKCI |
| | | RLVLEEQWYSSRNQMEIFTHLNIKPKKINLTAANKIPKAMIDEFILSPVVKRTFGQAI |
| | | NLINKIIEKYGVPEDIIIELARENNSKDKQKFINEMQKKNENTRKRINEIIGKYGNQNA |
| | | KRLVEKIRLHDEQEGKCLYSLESIPLEDLLNNPNHYEVDHIIPRSVSFDNSYHNKVLV |
| | | KQSENSKKSNLTPYQYFNSGKSKLSYNQFKQHILNLSKSQDRISKKKKEYLLEERDI |
| | | NKFEVQKEFINRNLVDTRYATRELTNYLKAYFSANNMNVKVKTINGSFTDYLRKV |
| | | WKFKKERNHGYKHHAEDALIIANADFLFKENKKLKAVNSVLEKPEIESKQLDIQVD |
| | | SEDNYSEMFIIPKQVQDIKDFRNFKYSHRVDKKPNRQLINDTLYSTRKKDNSTYIVQ |
| | | TIKDIYAKDNTTLKKQFDKSPEKFLMYQHDPRTFEKLEVIMKQYANEKNPLAKYHE |
| | | ETGEYLTKYSKKNNGPIVKSLKYIGNKLGSHLDVTHQFKSSTKKLVKLSIKPYRFDV |
| | | YLTDKGYKFITISYLDVLKKDNYYYIPEQKYDKLKLGKAIDKNAKFIASFYKNDLIK |
| | | LDGEIYKIIGVNSDTRNMIELDLPDIRYKEYCELNNIKGEPRIKKTIGKKVNSIEKLTT |
| | | DVLGNVFTNTQYTKPQLLFKRGN |
| 596 | SluCas9 | MNQKFILGLDIGITSVGYGLIDYETKNIIDAGVRLFPEANVENNEGRRSKRGSRRLKR |
| | N582A nickase | RRIHRLERVKKLLEDYNLLDQSQIPQSTNPYAIRVKGLSEALSKDELVIALLHIAKRR |
| | | GIHKIDVIDSNDDVGNELSTKEQLNKNSKLLKDKFVCQIQLERMNEGQVRGEKNRF |
| | | KTADIIKEIIQLLNVQKNFHQLDENFINKYIELVEMRREYFEGPGKGSPYGWEGDPK |
| | | AWYETLMGHCTYFPDELRSVKYAYSADLFNALNDLNNLVIQRDGLSKLEYHEKYH |
| | | IIENVFKQKKKPTLKQIANEINVNPEDIKGYRITKSGKPQFTEFKLYHDLKSVLFDQSI |
| | | LENEDVLDQIAEILTIYQDKDSIKSKLTELDILLNEEDKENIAQLTGYTGTHRLSLKCI |
| | | RLVLEEQWYSSRNQMEIFTHLNIKPKKINLTAANKIPKAMIDEFILSPVVKRTFGQAI |
| | | NLINKIIEKYGVPEDIIIELARENNSKDKQKFINEMQKKNENTRKRINEIIGKYGNQNA |
| | | KRLVEKIRLHDEQEGKCLYSLESIPLEDLLNNPNHYEVDHIIPRSVSFDNSYHNKVLV |
| | | KQSEASKKSNLTPYQYFNSGKSKLSYNQFKQHILNLSKSQDRISKKKKEYLLEERDI |
| | | NKFEVQKEFINRNLVDTRYATRELTNYLKAYFSANNMNVKVKTINGSFTDYLRKV |
| | | WKFKKERNHGYKHHAEDALIIANADFLFKENKKLKAVNSVLEKPEIESKQLDIQVD |
| | | SEDNYSEMFIIPKQVQDIKDFRNFKYSHRVDKKPNRQLINDTLYSTRKKDNSTYIVQ |
| | | TIKDIYAKDNTTLKKQFDKSPEKFLMYQHDPRTFEKLEVIMKQYANEKNPLAKYHE |
| | | ETGEYLTKYSKKNNGPIVKSLKYIGNKLGSHLDVTHQFKSSTKKLVKLSIKPYRFDV |
| | | YLTDKGYKFITISYLDVLKKDNYYYIPEQKYDKLKLGKAIDKNAKFIASFYKNDLIK |
| | | LDGEIYKIIGVNSDTRNMIELDLPDIRYKEYCELNNIKGEPRIKKTIGKKVNSIEKLTT |
| | | DVLGNVFTNTQYTKPQLLFKRGN |
| 597 | Met(−) | NQKFILGLDIGITSVGYGLIDYETKNIIDAGVRLFPEANVENNEGRRSKRGSRRLKRR |
| | SluCas9 | RIHRLERVKKLLEDYNLLDQSQIPQSTNPYAIRVKGLSEALSKDELVIALLHIAKRRGI |
| | nickase | HKIDVIDSNDDVGNELSTKEQLNKNSKLLKDKFVCQIQLERMNEGQVRGEKNRFKT |
| | | ADIIKEIIQLLNVQKNFHQLDENFINKYIELVEMRREYFEGPGKGSPYGWEGDPKAW |
| | | YETLMGHCTYFPDELRSVKYAYSADLFNALNDLNNLVIQRDGLSKLEYHEKYHIIE |
| | | NVFKQKKKPTLKQIANEINVNPEDIKGYRITKSGKPQFTEFKLYHDLKSVLFDQSILE |
| | | NEDVLDQIAEILTIYQDKDSIKSKLTELDILLNEEDKENIAQLTGYTGTHRLSLKCIRL |
| | | VLEEQWYSSRNQMEIFTHLNIKPKKINLTAANKIPKAMIDEFILSPVVKRTFGQAINLI |
| | | NKIIEKYGVPEDIIIELARENNSKDKQKFINEMQKKNENTRKRINEIIGKYGNQNAKR |
| | | LVEKIRLHDEQEGKCLYSLESIPLEDLLNNPNHYEVDHIIPRSVSFDNSYHNKVLVKQ |
| | | SEASKKSNLTPYQYFNSGKSKLSYNQFKQHILNLSKSQDRISKKKKEYLLEERDINKF |
| | | EVQKEFINRNLVDTRYATRELTNYLKAYFSANNMNVKVKTINGSFTDYLRKVWKF |
| | | KKERNHGYKHHAEDALIIANADFLFKENKKLKAVNSVLEKPEIESKQLDIQVDSEDN |
| | | YSEMFIIPKQVQDIKDFRNFKYSHRVDKKPNRQLINDTLYSTRKKDNSTYIVQTIKDI |
| | | YAKDNTTLKKQFDKSPEKFLMYQHDPRTFEKLEVIMKQYANEKNPLAKYHEETGE |
| | | YLTKYSKKNNGPIVKSLKYIGNKLGSHLDVTHQFKSSTKKLVKLSIKPYRFDVYLTD |
| | | KGYKFITISYLDVLKKDNYYYIPEQKYDKLKLGKAIDKNAKFIASFYKNDLIKLDGEI |
| | | YKIIGVNSDTRNMIELDLPDIRYKEYCELNNIKGEPRIKKTIGKKVNSIEKLTTDVLGN |
| | | VFTNTQYTKPQLLFKRGN |
| 598 | Staphylococcus | MKRNYILGLDIGITSVGYGIIDYETRDVIDAGVRLFKEANVENNEGRRSKRGARRLK |
| | aureus Cas9 | RRRRHRIQRVKKLLFDYNLLTDHSELSGINPYEARVKGLSQKLSEEEFSAALLHLAK |
| | (SaCas9) | RRGVHNVNEVEEDTGNELSTKEQISRNSKALEEKYVAELQLERLKKDGEVRGSINR |
| | | FKTSDYVKEAKQLLKVQKAYHQLDQSFIDTYIDLLETRRTYYEGPGEGSPFGWKDI |
| | | KEWYEMLMGHCTYFPEELRSVKYAYNADLYNALNDLNNLVITRDENEKLEYYEKF |
| | | QIIENVFKQKKKPTLKQIAKEILVNEEDIKGYRVTSTGKPEFTNLKVYHDIKDITARK |
| | | EIIENAELLDQIAKILTIYQSSEDIQEELTNLNSELTQEEIEQISNLKGYTGTHNLSLKAI |
| | | NLILDELWHTNDNQIAIFNRLKLVPKKVDLSQQKEIPTTLVDDFILSPVVKRSFIQSIK |
| | | VINAIIKKYGLPNDIIIELAREKNSKDAQKMINEMQKRNRQTNERIEEIIRTTGKENAK |
| | | YLIEKIKLHDMQEGKCLYSLEAIPLEDLLNNPFNYEVDHIIPRSVSFDNSFNNKVLVK |
| | | QEENSKKGNRTPFQYLSSSDSKISYETFKKHILNLAKGKGRISKTKKEYLLEERDINR |
| | | FSVQKDFINRNLVDTRYATRGLMNLLRSYFRVNNLDVKVKSINGGFTSFLRRKWKF |
| | | KKERNKGYKHHAEDALIIANADFIFKEWKKLDKAKKVMENQMFEEKQAESMPEIE |
| | | TEQEYKEIFITPHQIKHIKDFKDYKYSHRVDKKPNRELINDTLYSTRKDDKGNTLIVN |
| | | NLNGLYDKDNDKLKKLINKSPEKLLMYHHDPQTYQKLKLIMEQYGDEKNPLYKYY |
| | | EETGNYLTKYSKKDNGPVIKKIKYYGNKLNAHLDITDDYPNSRNKVVKLSLKPYRF |
| | | DVYLDNGVYKFVTVKNLDVIKKENYYEVNSKCYEEAKKLKKISNQAEFIASFYNND |
| | | LIKINGELYRVIGVNNDLLNRIEVNMIDITYREYLENMNDKRPPRIIKTIASKTQSIKK |
| | | YSTDILGNLYEVKSKKHPQIIKKG |
| 599 | SaCas9 N580A | MKRNYILGLDIGITSVGYGIIDYETRDVIDAGVRLFKEANVENNEGRRSKRGARRLK |
| | nickase | RRRRHRIQRVKKLLFDYNLLTDHSELSGINPYEARVKGLSQKLSEEEFSAALLHLAK |
| | | RRGVHNVNEVEEDTGNELSTKEQISRNSKALEEKYVAELQLERLKKDGEVRGSINR |
| | | FKTSDYVKEAKQLLKVQKAYHQLDQSFIDTYIDLLETRRTYYEGPGEGSPFGWKDI |
| | | KEWYEMLMGHCTYFPEELRSVKYAYNADLYNALNDLNNLVITRDENEKLEYYEKF |
| | | QIIENVFKQKKKPTLKQIAKEILVNEEDIKGYRVTSTGKPEFTNLKVYHDIKDITARK |
| | | EIIENAELLDQIAKILTIYQSSEDIQEELTNLNSELTQEEIEQISNLKGYTGTHNLSLKAI |
| | | NLILDELWHTNDNQIAIFNRLKLVPKKVDLSQQKEIPTTLVDDFILSPVVKRSFIQSIK |
| | | VINAIIKKYGLPNDIIIELAREKNSKDAQKMINEMQKRNRQTNERIEEIIRTTGKENAK |
| | | YLIEKIKLHDMQEGKCLYSLEAIPLEDLLNNPFNYEVDHIIPRSVSFDNSFNNKVLVK |
| | | QEEASKKGNRTPFQYLSSSDSKISYETFKKHILNLAKGKGRISKTKKEYLLEERDINR |
| | | FSVQKDFINRNLVDTRYATRGLMNLLRSYFRVNNLDVKVKSINGGFTSFLRRKWKF |
| | | KKERNKGYKHHAEDALIIANADFIFKEWKKLDKAKKVMENQMFEEKQAESMPEIE |
| | | TEQEYKEIFITPHQIKHIKDFKDYKYSHRVDKKPNRELINDTLYSTRKDDKGNTLIVN |
| | | NLNGLYDKDNDKLKKLINKSPEKLLMYHHDPQTYQKLKLIMEQYGDEKNPLYKYY |
| | | EETGNYLTKYSKKDNGPVIKKIKYYGNKLNAHLDITDDYPNSRNKVVKLSLKPYRF |
| | | DVYLDNGVYKFVTVKNLDVIKKENYYEVNSKCYEEAKKLKKISNQAEFIASFYNND |
| | | LIKINGELYRVIGVNNDLLNRIEVNMIDITYREYLENMNDKRPPRIIKTIASKTQSIKK |
| | | YSTDILGNLYEVKSKKHPQIIKKG |
| 600 | Met(−) SaCas9 | KRNYILGLDIGITSVGYGIIDYETRDVIDAGVRLFKEANVENNEGRRSKRGARRLKR |
| | nickase | RRRHRIQRVKKLLFDYNLLTDHSELSGINPYEARVKGLSQKLSEEEFSAALLHLAKR |
| | | RGVHNVNEVEEDTGNELSTKEQISRNSKALEEKYVAELQLERLKKDGEVRGSINRF |
| | | KTSDYVKEAKQLLKVQKAYHQLDQSFIDTYIDLLETRRTYYEGPGEGSPFGWKDIK |
| | | EWYEMLMGHCTYFPEELRSVKYAYNADLYNALNDLNNLVITRDENEKLEYYEKFQ |
| | | IIENVFKQKKKPTLKQIAKEILVNEEDIKGYRVTSTGKPEFTNLKVYHDIKDITARKEI |
| | | IENAELLDQIAKILTIYQSSEDIQEELTNLNSELTQEEIEQISNLKGYTGTHNLSLKAIN |
| | | LILDELWHTNDNQIAIFNRLKLVPKKVDLSQQKEIPTTLVDDFILSPVVKRSFIQSIKVI |
| | | NAIIKKYGLPNDIIIELAREKNSKDAQKMINEMQKRNRQTNERIEEIIRTTGKENAKY |
| | | LIEKIKLHDMQEGKCLYSLEAIPLEDLLNNPFNYEVDHIIPRSVSFDNSFNNKVLVKQ |
| | | EEASKKGNRTPFQYLSSSDSKISYETFKKHILNLAKGKGRISKTKKEYLLEERDINRFS |
| | | VQKDFINRNLVDTRYATRGLMNLLRSYFRVNNLDVKVKSINGGFTSFLRRKWKFK |
| | | KERNKGYKHHAEDALIIANADFIFKEWKKLDKAKKVMENQMFEEKQAESMPEIETE |
| | | QEYKEIFITPHQIKHIKDFKDYKYSHRVDKKPNRELINDTLYSTRKDDKGNTLIVNNL |
| | | NGLYDKDNDKLKKLINKSPEKLLMYHHDPQTYQKLKLIMEQYGDEKNPLYKYYEE |
| | | TGNYLTKYSKKDNGPVIKKIKYYGNKLNAHLDITDDYPNSRNKVVKLSLKPYRFDV |
| | | YLDNGVYKFVTVKNLDVIKKENYYEVNSKCYEEAKKLKKISNQAEFIASFYNNDLI |
| | | KINGELYRVIGVNNDLLNRIEVNMIDITYREYLENMNDKRPPRIIKTIASKTQSIKKYS |
| | | TDILGNLYEVKSKKHPQIIKKG |
| 601 | SpCas9-NG | MDKKYSIGLDIGTNSVGWAVITDEYKVPSKKFKVLGNTDRHSIKKNLIGALLFDSGE |
| | (VRVRFRR) | TAEATRLKRTARRRYTRRKNRICYLQEIFSNEMAKVDDSFFHRLEESFLVEEDKKHE |
| | | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDHIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESIRPKRNSDKLIARKKDWDPKKYGGFVSPTV |
| | | AYSVLVVAKVEKGKSKKLKSVKELLGITIMERSSFEKNPIDFLEAKGYKEVKKDLIIK |
| | | LPKYSLFELENGRKRMLASARFLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPRAFKYFDTTIDRKVYRSTKEVLDATLIHQSITGLYETRIDLSQLGGD |
| 602 | spCas9-NG | MDKKYSIGLDIGTNSVGWAVITDEYKVPSKKFKVLGNTDRHSIKKNLIGALLFDSGE |
| | (H840A_VRVRFRR) | TAEATRLKRTARRRYTRRKNRICYLQEIFSNEMAKVDDSFFHRLEESFLVEEDKKHE |
| | Nickase | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESIRPKRNSDKLIARKKDWDPKKYGGFVSPTV |
| | | AYSVLVVAKVEKGKSKKLKSVKELLGITIMERSSFEKNPIDFLEAKGYKEVKKDLIIK |
| | | LPKYSLFELENGRKRMLASARFLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPRAFKYFDTTIDRKVYRSTKEVLDATLIHQSITGLYETRIDLSQLGGD |
| 603 | Met(−) | DKKYSIGLDIGTNSVGWAVITDEYKVPSKKFKVLGNTDRHSIKKNLIGALLFDSGET |
| | SpCas9-NG | AEATRLKRTARRRYTRRKNRICYLQEIFSNEMAKVDDSFFHRLEESFLVEEDKKHER |
| | Nickase | HPIFGNIVDEVAYHEKYPTIYHLRKKLVDSTDKADLRLIYLALAHMIKFRGHFLIEGD |
| | | LNPDNSDVDKLFIQLVQTYNQLFEENPINASGVDAKAILSARLSKSRRLENLIAQLPG |
| | | EKKNGLFGNLIALSLGLTPNFKSNFDLAEDAKLQLSKDTYDDDLDNLLAQIGDQYA |
| | | DLFLAAKNLSDAILLSDILRVNTEITKAPLSASMIKRYDEHHQDLTLLKALVRQQLPE |
| | | KYKEIFFDQSKNGYAGYIDGGASQEEFYKFIKPILEKMDGTEELLVKLNREDLLRKQ |
| | | RTFDNGSIPHQIHLGELHAILRRQEDFYPFLKDNREKIEKILTFRIPYYVGPLARGNSR |
| | | FAWMTRKSEETITPWNFEEVVDKGASAQSFIERMTNFDKNLPNEKVLPKHSLLYEY |
| | | FTVYNELTKVKYVTEGMRKPAFLSGEQKKAIVDLLFKTNRKVTVKQLKEDYFKKIE |
| | | CFDSVEISGVEDRFNASLGTYHDLLKIIKDKDFLDNEENEDILEDIVLTLTLFEDREMI |
| | | EERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESIRPKRNSDKLIARKKDWDPKKYGGFVSPTV |
| | | AYSVLVVAKVEKGKSKKLKSVKELLGITIMERSSFEKNPIDFLEAKGYKEVKKDLIIK |
| | | LPKYSLFELENGRKRMLASARFLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPRAFKYFDTTIDRKVYRSTKEVLDATLIHQSITGLYETRIDLSQLGGD |
| 604 | spCas9-NGA | MDKKYSIGLDIGTNSVGWAVITDEYKVPSKKFKVLGNTDRHSIKKNLIGALLFDSGE |
| | (VRQR) | TAEATRLKRTARRRYTRRKNRICYLQEIFSNEMAKVDDSFFHRLEESFLVEEDKKHE |
| | | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDHIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFVSPTV |
| | | AYSVLVVAKVEKGKSKKLKSVKELLGITIMERSSFEKNPIDFLEAKGYKEVKKDLIIK |
| | | LPKYSLFELENGRKRMLASARELQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKQYRSTKEVLDATLIHQSITGLYETRIDLSQLGGD |
| 605 | spCas9-NGA | MDKKYSIGLDIGTNSVGWAVITDEYKVPSKKFKVLGNTDRHSIKKNLIGALLFDSGE |
| | (H840A_VRQR) | TAEATRLKRTARRRYTRRKNRICYLQEIFSNEMAKVDDSFFHRLEESFLVEEDKKHE |
| | Nickase | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFVSPTV |
| | | AYSVLVVAKVEKGKSKKLKSVKELLGITIMERSSFEKNPIDFLEAKGYKEVKKDLIIK |
| | | LPKYSLFELENGRKRMLASARELQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKQYRSTKEVLDATLIHQSITGLYETRIDLSQLGGD |
| 606 | Met(−) spCas9- | DKKYSIGLDIGTNSVGWAVITDEYKVPSKKFKVLGNTDRHSIKKNLIGALLFDSGET |
| | NGA Nickase | AEATRLKRTARRRYTRRKNRICYLQEIFSNEMAKVDDSFFHRLEESFLVEEDKKHER |
| | | HPIFGNIVDEVAYHEKYPTIYHLRKKLVDSTDKADLRLIYLALAHMIKFRGHFLIEGD |
| | | LNPDNSDVDKLFIQLVQTYNQLFEENPINASGVDAKAILSARLSKSRRLENLIAQLPG |
| | | EKKNGLFGNLIALSLGLTPNFKSNFDLAEDAKLQLSKDTYDDDLDNLLAQIGDQYA |
| | | DLFLAAKNLSDAILLSDILRVNTEITKAPLSASMIKRYDEHHQDLTLLKALVRQQLPE |
| | | KYKEIFFDQSKNGYAGYIDGGASQEEFYKFIKPILEKMDGTEELLVKLNREDLLRKQ |
| | | RTFDNGSIPHQIHLGELHAILRRQEDFYPFLKDNREKIEKILTFRIPYYVGPLARGNSR |
| | | FAWMTRKSEETITPWNFEEVVDKGASAQSFIERMTNFDKNLPNEKVLPKHSLLYEY |
| | | FTVYNELTKVKYVTEGMRKPAFLSGEQKKAIVDLLFKTNRKVTVKQLKEDYFKKIE |
| | | CFDSVEISGVEDRFNASLGTYHDLLKIIKDKDFLDNEENEDILEDIVLTLTLFEDREMI |
| | | EERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFVSPTV |
| | | AYSVLVVAKVEKGKSKKLKSVKELLGITIMERSSFEKNPIDFLEAKGYKEVKKDLIIK |
| | | LPKYSLFELENGRKRMLASARELQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKQYRSTKEVLDATLIHQSITGLYETRIDLSQLGGD |
| 607 | SpRY Cas9 | MDKKYSIGLDIGTNSVGWAVITDEYKVPSKKFKVLGNTDRHSIKKNLIGALLFDSGE |
| | | TAERTRLKRTARRRYTRRKNRICYLQEIFSNEMAKVDDSFFHRLEESFLVEEDKKHE |
| | | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDHIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESIRPKRNSDKLIARKKDWDPKKYGGFLWPTV |
| | | AYSVLVVAKVEKGKSKKLKSVKELLGITIMERSSFEKNPIDFLEAKGYKEVKKDLIIK |
| | | LPKYSLFELENGRKRMLASAKQLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTRLGAPRAFKYFDTTIDPKQYRSTKEVLDATLIHQSITGLYETRIDLSQLGGD |
| 608 | SpRY Cas9 | MDKKYSIGLDIGTNSVGWAVITDEYKVPSKKFKVLGNTDRHSIKKNLIGALLFDSGE |
| | (H840A) | TAERTRLKRTARRRYTRRKNRICYLQEIFSNEMAKVDDSFFHRLEESFLVEEDKKHE |
| | Nickase | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESIRPKRNSDKLIARKKDWDPKKYGGFLWPTV |
| | | AYSVLVVAKVEKGKSKKLKSVKELLGITIMERSSFEKNPIDFLEAKGYKEVKKDLIIK |
| | | LPKYSLFELENGRKRMLASAKQLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTRLGAPRAFKYFDTTIDPKQYRSTKEVLDATLIHQSITGLYETRIDLSQLGGD |
| 609 | Met(−) SpRY | DKKYSIGLDIGTNSVGWAVITDEYKVPSKKFKVLGNTDRHSIKKNLIGALLFDSGET |
| | Cas9 Nickase | AERTRLKRTARRRYTRRKNRICYLQEIFSNEMAKVDDSFFHRLEESFLVEEDKKHER |
| | | HPIFGNIVDEVAYHEKYPTIYHLRKKLVDSTDKADLRLIYLALAHMIKFRGHFLIEGD |
| | | LNPDNSDVDKLFIQLVQTYNQLFEENPINASGVDAKAILSARLSKSRRLENLIAQLPG |
| | | EKKNGLFGNLIALSLGLTPNFKSNFDLAEDAKLQLSKDTYDDDLDNLLAQIGDQYA |
| | | DLFLAAKNLSDAILLSDILRVNTEITKAPLSASMIKRYDEHHQDLTLLKALVRQQLPE |
| | | KYKEIFFDQSKNGYAGYIDGGASQEEFYKFIKPILEKMDGTEELLVKLNREDLLRKQ |
| | | RTFDNGSIPHQIHLGELHAILRRQEDFYPFLKDNREKIEKILTFRIPYYVGPLARGNSR |
| | | FAWMTRKSEETITPWNFEEVVDKGASAQSFIERMTNFDKNLPNEKVLPKHSLLYEY |
| | | FTVYNELTKVKYVTEGMRKPAFLSGEQKKAIVDLLFKTNRKVTVKQLKEDYFKKIE |
| | | CFDSVEISGVEDRFNASLGTYHDLLKIIKDKDFLDNEENEDILEDIVLTLTLFEDREMI |
| | | EERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESIRPKRNSDKLIARKKDWDPKKYGGFLWPTV |
| | | AYSVLVVAKVEKGKSKKLKSVKELLGITIMERSSFEKNPIDFLEAKGYKEVKKDLIIK |
| | | LPKYSLFELENGRKRMLASAKQLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTRLGAPRAFKYFDTTIDPKQYRSTKEVLDATLIHQSITGLYETRIDLSQLGGD |
| 610 | SRGN3.1 | MNQKFILGLDIGITSVGYGLIDYETKNIIDAGVRLFPEANVENNEGRRSKRGSRRLKR |
| | | RRIHRLERVKLLLTEYDLINKEQIPTSNNPYQIRVKGLSEILSKDELAIALLHLAKRRG |
| | | IHNVDVAADKEETASDSLSTKDQINKNAKFLESRYVCELQKERLENEGHVRGVENR |
| | | FLTKDIVREAKKIIDTQMQYYPEIDETFKEKYISLVETRREYFEGPGQGSPFGWNGDL |
| | | KKWYEMLMGHCTYFPQELRSVKYAYSADLFNALNDLNNLIIQRDNSEKLEYHEKY |
| | | HIIENVFKQKKKPTLKQIAKEIGVNPEDIKGYRITKSGTPEFTSFKLFHDLKKVVKDH |
| | | AILDDIDLLNQIAEILTIYQDKDSIVAELGQLEYLMSEADKQSISELTGYTGTHSLSLK |
| | | CMNMIIDELWHSSMNQMEVFTYLNMRPKKYELKGYQRIPTDMIDDAILSPVVKRTF |
| | | IQSINVINKVIEKYGIPEDIIIELARENNSDDRKKFINNLQKKNEATRKRINEIIGQTGN |
| | | QNAKRIVEKIRLHDQQEGKCLYSLESIPLEDLLNNPNHYEVDHIIPRSVSFDNSYHNK |
| | | VLVKQSENSKKSNLTPYQYFNSGKSKLSYNQFKQHILNLSKSQDRISKKKKEYLLEE |
| | | RDINKFEVQKEFINRNLVDTRYATRELTNYLKAYFSANNMNVKVKTINGSFTDYLR |
| | | KVWKFKKERNHGYKHHAEDALIIANADFLFKENKKLKAVNSVLEKPEIETKQLDIQ |
| | | VDSEDNYSEMFIIPKQVQDIKDFRNFKYSHRVDKKPNRQLINDTLYSTRKKDNSTYI |
| | | VQTIKDIYAKDNTTLKKQFDKSPEKFLMYQHDPRTFEKLEVIMKQYANEKNPLAKY |
| | | HEETGEYLTKYSKKNNGPIVKSLKYIGNKLGSHLDVTHQFKSSTKKLVKLSIKNYRF |
| | | DVYLTEKGYKFVTIAYLNVFKKDNYYYIPKDKYQELKEKKKIKDTDQFIASFYKND |
| | | LIKLNGDLYKIIGVNSDDRNIIELDYYDIKYKDYCEINNIKGEPRIKKTIGKKTESIEKF |
| | | TTDVLGNLYLHSTEKAPQLIFKRGL |
| 611 | SRGN3.1 | MNQKFILGLDIGITSVGYGLIDYETKNIIDAGVRLFPEANVENNEGRRSKRGSRRLKR |
| | (N585A) | RRIHRLERVKLLLTEYDLINKEQIPTSNNPYQIRVKGLSEILSKDELAIALLHLAKRRG |
| | Nickase | IHNVDVAADKEETASDSLSTKDQINKNAKFLESRYVCELQKERLENEGHVRGVENR |
| | | FLTKDIVREAKKIIDTQMQYYPEIDETFKEKYISLVETRREYFEGPGQGSPFGWNGDL |
| | | KKWYEMLMGHCTYFPQELRSVKYAYSADLFNALNDLNNLIIQRDNSEKLEYHEKY |
| | | HIIENVFKQKKKPTLKQIAKEIGVNPEDIKGYRITKSGTPEFTSFKLFHDLKKVVKDH |
| | | AILDDIDLLNQIAEILTIYQDKDSIVAELGQLEYLMSEADKQSISELTGYTGTHSLSLK |
| | | CMNMIIDELWHSSMNQMEVFTYLNMRPKKYELKGYQRIPTDMIDDAILSPVVKRTF |
| | | IQSINVINKVIEKYGIPEDIIIELARENNSDDRKKFINNLQKKNEATRKRINEIIGQTGN |
| | | QNAKRIVEKIRLHDQQEGKCLYSLESIPLEDLLNNPNHYEVDHIIPRSVSFDNSYHNK |
| | | VLVKQSEASKKSNLTPYQYFNSGKSKLSYNQFKQHILNLSKSQDRISKKKKEYLLEE |
| | | RDINKFEVQKEFINRNLVDTRYATRELTNYLKAYFSANNMNVKVKTINGSFTDYLR |
| | | KVWKFKKERNHGYKHHAEDALIIANADFLFKENKKLKAVNSVLEKPEIETKQLDIQ |
| | | VDSEDNYSEMFIIPKQVQDIKDFRNFKYSHRVDKKPNRQLINDTLYSTRKKDNSTYI |
| | | VQTIKDIYAKDNTTLKKQFDKSPEKFLMYQHDPRTFEKLEVIMKQYANEKNPLAKY |
| | | HEETGEYLTKYSKKNNGPIVKSLKYIGNKLGSHLDVTHQFKSSTKKLVKLSIKNYRF |
| | | DVYLTEKGYKFVTIAYLNVFKKDNYYYIPKDKYQELKEKKKIKDTDQFIASFYKND |
| | | LIKLNGDLYKIIGVNSDDRNIIELDYYDIKYKDYCEINNIKGEPRIKKTIGKKTESIEKF |
| | | TTDVLGNLYLHSTEKAPQLIFKRGL |
| 612 | Met(−) | NQKFILGLDIGITSVGYGLIDYETKNIIDAGVRLFPEANVENNEGRRSKRGSRRLKRR |
| | SRGN3.1 (N584A) | RIHRLERVKLLLTEYDLINKEQIPTSNNPYQIRVKGLSEILSKDELAIALLHLAKRRGI |
| | Nickase | HNVDVAADKEETASDSLSTKDQINKNAKFLESRYVCELQKERLENEGHVRGVENRF |
| | | LTKDIVREAKKIIDTQMQYYPEIDETFKEKYISLVETRREYFEGPGQGSPFGWNGDLK |
| | | KWYEMLMGHCTYFPQELRSVKYAYSADLFNALNDLNNLIIQRDNSEKLEYHEKYHI |
| | | IENVFKQKKKPTLKQIAKEIGVNPEDIKGYRITKSGTPEFTSFKLFHDLKKVVKDHAI |
| | | LDDIDLLNQIAEILTIYQDKDSIVAELGQLEYLMSEADKQSISELTGYTGTHSLSLKC |
| | | MNMIIDELWHSSMNQMEVFTYLNMRPKKYELKGYQRIPTDMIDDAILSPVVKRTFI |
| | | QSINVINKVIEKYGIPEDIIIELARENNSDDRKKFINNLQKKNEATRKRINEIIGQTGNQ |
| | | NAKRIVEKIRLHDQQEGKCLYSLESIPLEDLLNNPNHYEVDHIIPRSVSFDNSYHNKV |
| | | LVKQSEASKKSNLTPYQYFNSGKSKLSYNQFKQHILNLSKSQDRISKKKKEYLLEER |
| | | DINKFEVQKEFINRNLVDTRYATRELTNYLKAYFSANNMNVKVKTINGSFTDYLRK |
| | | VWKFKKERNHGYKHHAEDALIIANADFLFKENKKLKAVNSVLEKPEIETKQLDIQV |
| | | DSEDNYSEMFIIPKQVQDIKDFRNFKYSHRVDKKPNRQLINDTLYSTRKKDNSTYIV |
| | | QTIKDIYAKDNTTLKKQFDKSPEKFLMYQHDPRTFEKLEVIMKQYANEKNPLAKYH |
| | | EETGEYLTKYSKKNNGPIVKSLKYIGNKLGSHLDVTHQFKSSTKKLVKLSIKNYRFD |
| | | VYLTEKGYKFVTIAYLNVFKKDNYYYIPKDKYQELKEKKKIKDTDQFIASFYKNDLI |
| | | KLNGDLYKIIGVNSDDRNIIELDYYDIKYKDYCEINNIKGEPRIKKTIGKKTESIEKFTT |
| | | DVLGNLYLHSTEKAPQLIFKRGL |
| 613 | SRGN3.3 | MNQKFILGLDIGITSVGYGLIDYETKNIIDAGVRLFPEANVENNEGRRSKRGSRRLKR |
| | | RRIHRLERVKLLLTEYDLINKEQIPTSNNPYQIRVKGLSEILSKDELAIALLHLAKRRG |
| | | IHNVDVAADKEETASDSLSTKDQINKNAKFLESRYVCELQKERLENEGHVRGVENR |
| | | FLTKDIVREAKKIIDTQMQYYPEIDETFKEKYISLVETRREYFEGPGQGSPFGWNGDL |
| | | KKWYEMLMGHCTYFPQELRSVKYAYSADLFNALNDLNNLIIQRDNSEKLEYHEKY |
| | | HIIENVFKQKKKPTLKQIAKEIGVNPEDIKGYRITKSGTPEFTSFKLFHDLKKVVKDH |
| | | AILDDIDLLNQIAEILTIYQDKDSIVAELGQLEYLMSEADKQSISELTGYTGTHSLSLK |
| | | CMNMIIDELWHSSMNQMEVFTYLNMRPKKYELKGYQRIPTDMIDDAILSPVVKRTF |
| | | IQSINVINKVIEKYGIPEDIIIELARENNSDDRKKFINNLQKKNEATRKRINEIIGQTGN |
| | | QNAKRIVEKIRLHDQQEGKCLYSLESIPLEDLLNNPNHYEVDHIIPRSVSFDNSYHNK |
| | | VLVKQSENSKKSNLTPYQYFNSGKSKLSYNQFKQHILNLSKSQDRISKKKKEYLLEE |
| | | RDINKFEVQKEFINRNLVDTRYATRELTSYLKAYFSANNMDVKVKTINGSFTNHLR |
| | | KVWRFDKYRNHGYKHHAEDALIIANADFLFKENKKLQNTNKILEKPTIENNTKKVT |
| | | VEKEEDYNNVFETPKLVEDIKQYRDYKFSHRVDKKPNRQLINDTLYSTRMKDEHDY |
| | | IVQTITDIYGKDNTNLKKQFNKNPEKFLMYQNDPKTFEKLSIIMKQYSDEKNPLAKY |
| | | YEETGEYLTKYSKKNNGPIVKKIKLLGNKVGNHLDVTNKYENSTKKLVKLSIKNYR |
| | | FDVYLTEKGYKFVTIAYLNVFKKDNYYYIPKDKYQELKEKKKIKDTDQFIASFYKN |
| | | DLIKLNGDLYKIIGVNSDDRNIIELDYYDIKYKDYCEINNIKGEPRIKKTIGKKTESIEK |
| | | FTTDVLGNLYLHSTEKAPQLIFKRGL |
| 614 | SRGN3.3 (N585A) | MNQKFILGLDIGITSVGYGLIDYETKNIIDAGVRLFPEANVENNEGRRSKRGSRRLKR |
| | Nickase | RRIHRLERVKLLLTEYDLINKEQIPTSNNPYQIRVKGLSEILSKDELAIALLHLAKRRG |
| | | IHNVDVAADKEETASDSLSTKDQINKNAKFLESRYVCELQKERLENEGHVRGVENR |
| | | FLTKDIVREAKKIIDTQMQYYPEIDETFKEKYISLVETRREYFEGPGQGSPFGWNGDL |
| | | KKWYEMLMGHCTYFPQELRSVKYAYSADLFNALNDLNNLIIQRDNSEKLEYHEKY |
| | | HIIENVFKQKKKPTLKQIAKEIGVNPEDIKGYRITKSGTPEFTSFKLFHDLKKVVKDH |
| | | AILDDIDLLNQIAEILTIYQDKDSIVAELGQLEYLMSEADKQSISELTGYTGTHSLSLK |
| | | CMNMIIDELWHSSMNQMEVFTYLNMRPKKYELKGYQRIPTDMIDDAILSPVVKRTF |
| | | IQSINVINKVIEKYGIPEDIIIELARENNSDDRKKFINNLQKKNEATRKRINEIIGQTGN |
| | | QNAKRIVEKIRLHDQQEGKCLYSLESIPLEDLLNNPNHYEVDHIIPRSVSFDNSYHNK |
| | | VLVKQSEASKKSNLTPYQYFNSGKSKLSYNQFKQHILNLSKSQDRISKKKKEYLLEE |
| | | RDINKFEVQKEFINRNLVDTRYATRELTSYLKAYFSANNMDVKVKTINGSFTNHLR |
| | | KVWRFDKYRNHGYKHHAEDALIIANADFLFKENKKLQNTNKILEKPTIENNTKKVT |
| | | VEKEEDYNNVFETPKLVEDIKQYRDYKFSHRVDKKPNRQLINDTLYSTRMKDEHDY |
| | | IVQTITDIYGKDNTNLKKQFNKNPEKFLMYQNDPKTFEKLSIIMKQYSDEKNPLAKY |
| | | YEETGEYLTKYSKKNNGPIVKKIKLLGNKVGNHLDVTNKYENSTKKLVKLSIKNYR |
| | | FDVYLTEKGYKFVTIAYLNVFKKDNYYYIPKDKYQELKEKKKIKDTDQFIASFYKN |
| | | DLIKLNGDLYKIIGVNSDDRNIIELDYYDIKYKDYCEINNIKGEPRIKKTIGKKTESIEK |
| | | FTTDVLGNLYLHSTEKAPQLIFKRGL |
| 615 | Met(−) | NQKFILGLDIGITSVGYGLIDYETKNIIDAGVRLFPEANVENNEGRRSKRGSRRLKRR |
| | SRGN3.3 (N584A) | RIHRLERVKLLLTEYDLINKEQIPTSNNPYQIRVKGLSEILSKDELAIALLHLAKRRGI |
| | Nickase | HNVDVAADKEETASDSLSTKDQINKNAKFLESRYVCELQKERLENEGHVRGVENRF |
| | | LTKDIVREAKKIIDTQMQYYPEIDETFKEKYISLVETRREYFEGPGQGSPFGWNGDLK |
| | | KWYEMLMGHCTYFPQELRSVKYAYSADLFNALNDLNNLIIQRDNSEKLEYHEKYHI |
| | | IENVFKQKKKPTLKQIAKEIGVNPEDIKGYRITKSGTPEFTSFKLFHDLKKVVKDHAI |
| | | LDDIDLLNQIAEILTIYQDKDSIVAELGQLEYLMSEADKQSISELTGYTGTHSLSLKC |
| | | MNMIIDELWHSSMNQMEVFTYLNMRPKKYELKGYQRIPTDMIDDAILSPVVKRTFI |
| | | QSINVINKVIEKYGIPEDIIIELARENNSDDRKKFINNLQKKNEATRKRINEIIGQTGNQ |
| | | NAKRIVEKIRLHDQQEGKCLYSLESIPLEDLLNNPNHYEVDHIIPRSVSFDNSYHNKV |
| | | LVKQSEASKKSNLTPYQYFNSGKSKLSYNQFKQHILNLSKSQDRISKKKKEYLLEER |
| | | DINKFEVQKEFINRNLVDTRYATRELTSYLKAYFSANNMDVKVKTINGSFTNHLRK |
| | | VWRFDKYRNHGYKHHAEDALIIANADFLFKENKKLQNTNKILEKPTIENNTKKVTV |
| | | EKEEDYNNVFETPKLVEDIKQYRDYKFSHRVDKKPNRQLINDTLYSTRMKDEHDYI |
| | | VQTITDIYGKDNTNLKKQFNKNPEKFLMYQNDPKTFEKLSIIMKQYSDEKNPLAKY |
| | | YEETGEYLTKYSKKNNGPIVKKIKLLGNKVGNHLDVTNKYENSTKKLVKLSIKNYR |
| | | FDVYLTEKGYKFVTIAYLNVFKKDNYYYIPKDKYQELKEKKKIKDTDQFIASFYKN |
| | | DLIKLNGDLYKIIGVNSDDRNIIELDYYDIKYKDYCEINNIKGEPRIKKTIGKKTESIEK |
| | | FTTDVLGNLYLHSTEKAPQLIFKRGL |
| 616 | SpG | MDKKYSIGLDIGTNSVGWAVITDEYKVPSKKFKVLGNTDRHSIKKNLIGALLFDSGE |
| | | TAEATRLKRTARRRYTRRKNRICYLQEIFSNEMAKVDDSFFHRLEESFLVEEDKKHE |
| | | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDHIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFLWPTV |
| | | AYSVLVVAKVEKGKSKKLKSVKELLGITIMERSSFEKNPIDFLEAKGYKEVKKDLIIK |
| | | LPKYSLFELENGRKRMLASAKQLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKQYRSTKEVLDATLIHQSITGLYETRIDLSQLGGD |
| 617 | SpG (H840A) | MDKKYSIGLDIGTNSVGWAVITDEYKVPSKKFKVLGNTDRHSIKKNLIGALLFDSGE |
| | Nickase | TAEATRLKRTARRRYTRRKNRICYLQEIFSNEMAKVDDSFFHRLEESFLVEEDKKHE |
| | | RHPIFGNIVDEVAYHEKYPTIYHLRKKLVDSTDKADLRLIYLALAHMIKFRGHFLIEG |
| | | DLNPDNSDVDKLFIQLVQTYNQLFEENPINASGVDAKAILSARLSKSRRLENLIAQLP |
| | | GEKKNGLFGNLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKLNREDLLRK |
| | | QRTFDNGSIPHQIHLGELHAILRRQEDFYPFLKDNREKIEKILTFRIPYYVGPLARGNS |
| | | RFAWMTRKSEETITPWNFEEVVDKGASAQSFIERMTNFDKNLPNEKVLPKHSLLYE |
| | | YFTVYNELTKVKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVLTLTLFEDREM |
| | | IEERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFLWPTV |
| | | AYSVLVVAKVEKGKSKKLKSVKELLGITIMERSSFEKNPIDFLEAKGYKEVKKDLIIK |
| | | LPKYSLFELENGRKRMLASAKQLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKQYRSTKEVLDATLIHQSITGLYETRIDLSQLGGD |
| 618 | Met(−) | DKKYSIGLDIGTNSVGWAVITDEYKVPSKKFKVLGNTDRHSIKKNLIGALLFDSGET |
| | SpG (H839A) | AEATRLKRTARRRYTRRKNRICYLQEIFSNEMAKVDDSFFHRLEESFLVEEDKKHER |
| | Nickase | HPIFGNIVDEVAYHEKYPTIYHLRKKLVDSTDKADLRLIYLALAHMIKFRGHFLIEGD |
| | | LNPDNSDVDKLFIQLVQTYNQLFEENPINASGVDAKAILSARLSKSRRLENLIAQLPG |
| | | EKKNGLFGNLIALSLGLTPNFKSNFDLAEDAKLQLSKDTYDDDLDNLLAQIGDQYA |
| | | DLFLAAKNLSDAILLSDILRVNTEITKAPLSASMIKRYDEHHQDLTLLKALVRQQLPE |
| | | KYKEIFFDQSKNGYAGYIDGGASQEEFYKFIKPILEKMDGTEELLVKLNREDLLRKQ |
| | | RTFDNGSIPHQIHLGELHAILRRQEDFYPFLKDNREKIEKILTFRIPYYVGPLARGNSR |
| | | FAWMTRKSEETITPWNFEEVVDKGASAQSFIERMTNFDKNLPNEKVLPKHSLLYEY |
| | | FTVYNELTKVKYVTEGMRKPAFLSGEQKKAIVDLLFKTNRKVTVKQLKEDYFKKIE |
| | | CFDSVEISGVEDRFNASLGTYHDLLKIIKDKDFLDNEENEDILEDIVLTLTLFEDREMI |
| | | EERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFLWPTV |
| | | AYSVLVVAKVEKGKSKKLKSVKELLGITIMERSSFEKNPIDFLEAKGYKEVKKDLIIK |
| | | LPKYSLFELENGRKRMLASAKQLQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKQYRSTKEVLDATLIHQSITGLYETRIDLSQLGGD |
| 619 | -CAS9 (R221K | DKKYSIGLDIGTNSVGWAVITDEYKVPSKKFKVLGNTDRHSIKKNLIGALLFDSGET |
| | N394K H840A) | AEATRLKRTARRRYTRRKNRICYLQEIFSNEMAKVDDSFFHRLEESFLVEEDKKHER |
| | | HPIFGNIVDEVAYHEKYPTIYHLRKKLVDSTDKADLRLIYLALAHMIKFRGHFLIEGD |
| | | LNPDNSDVDKLFIQLVQTYNQLFEENPINASGVDAKAILSARLSKSRKLENLIAQLPG |
| | | EKKNGLFGNLIALSLGLTPNFKSNFDLAEDAKLQLSKDTYDDDLDNLLAQIGDQYA |
| | | DLFLAAKNLSDAILLSDILRVNTEITKAPLSASMIKRYDEHHQDLTLLKALVRQQLPE |
| | | KYKEIFFDQSKNGYAGYIDGGASQEEFYKFIKPILEKMDGTEELLVKLKREDLLRKQ |
| | | RTFDNGSIPHQIHLGELHAILRRQEDFYPFLKDNREKIEKILTFRIPYYVGPLARGNSR |
| | | FAWMTRKSEETITPWNFEEVVDKGASAQSFIERMTNFDKNLPNEKVLPKHSLLYEY |
| | | FTVYNELTKVKYVTEGMRKPAFLSGEQKKAIVDLLFKTNRKVTVKQLKEDYFKKIE |
| | | CFDSVEISGVEDRFNASLGTYHDLLKIIKDKDFLDNEENEDILEDIVLTLTLFEDREMI |
| | | EERLKTYAHLFDDKVMKQLKRRRYTGWGRLSRKLINGIRDKQSGKTILDFLKSDGF |
| | | ANRNFMQLIHDDSLTFKEDIQKAQVSGQGDSLHEHIANLAGSPAIKKGILQTVKVVD |
| | | ELVKVMGRHKPENIVIEMARENQTTQKGQKNSRERMKRIEEGIKELGSQILKEHPVE |
| | | NTQLQNEKLYLYYLQNGRDMYVDQELDINRLSDYDVDAIVPQSFLKDDSIDNKVLT |
| | | RSDKNRGKSDNVPSEEVVKKMKNYWRQLLNAKLITQRKFDNLTKAERGGLSELDK |
| | | AGFIKRQLVETRQITKHVAQILDSRMNTKYDENDKLIREVKVITLKSKLVSDFRKDF |
| | | QFYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDFATV |
| | | RKVLSMPQVNIVKKTEVQTGGFSKESILPKRNSDKLIARKKDWDPKKYGGFDSPTV |
| | | AYSVLVVAKVEKGKSKKLKSVKELLGITIMERSSFEKNPIDFLEAKGYKEVKKDLIIK |
| | | LPKYSLFELENGRKRMLASAGELQKGNELALPSKYVNFLYLASHYEKLKGSPEDNE |
| | | QKQLFVEQHKHYLDEIIEQISEFSKRVILADANLDKVLSAYNKHRDKPIREQAENIIH |
| | | LFTLTNLGAPAAFKYFDTTIDRKRYTSTKEVLDATLIHQSITGLYETRIDLSQLGGD |

In some embodiments, a Cas9 protein comprises a variant Cas9 protein containing one or more amino acid substitutions. In some embodiments, a wildtype Cas9 protein comprises a RuvC domain and an HNH domain. In some embodiments, a prime editor comprises a nuclease active Cas9 protein that may cleave both strands of a double stranded target DNA sequence. In some embodiments, the nuclease active Cas9 protein comprises a functional RuvC domain and a functional HNH domain. In some embodiments, a prime editor comprises a Cas9 nickase that can bind to a guide polynucleotide and recognize a target DNA, but can cleave only one strand of a double stranded target DNA. In some embodiments, the Cas9 nickase comprises only one functional RuvC domain or one functional HNH domain. In some embodiments, a prime editor comprises a Cas9 that has a non-functional HNH domain and a functional RuvC domain. In some embodiments, the prime editor can cleave the edit strand (i.e., the PAM strand), but not the non-edit strand of a double stranded target DNA sequence. In some embodiments, a prime editor comprises a Cas9 having a non-functional RuvC domain that can cleave the target strand (i.e., the non-PAM strand), but not the edit strand of a double stranded target DNA sequence. In some embodiments, a prime editor comprises a Cas9 that has neither a functional RuvC domain nor a functional HNH domain, which may not cleave any strand of a double stranded target DNA sequence.

In some embodiments, a prime editor comprises a Cas9 having a mutation in the RuvC domain that reduces or abolishes the nuclease activity of the RuvC domain. In some embodiments, the Cas9 comprises a mutation at amino acid D10 as compared to a wild type SpCas9 as set forth in SEQ ID NO: 592, or a corresponding mutation thereof. In some embodiments, the Cas9 comprises a D10A mutation as compared to a wild type SpCas9 as set forth in SEQ ID NO: 592, or a corresponding mutation thereof. In some embodiments, the Cas9 polypeptide comprises a mutation at amino acid D10, G12, and/or G17 as compared to a wild type SpCas9 as set forth in SEQ ID NO: 592, or a corresponding mutation thereof. In some embodiments, the Cas9 polypeptide comprises a D10A mutation, a G12A mutation, and/or a G17A mutation as compared to a wild type SpCas9 as set forth in SEQ ID NO: 592, or a corresponding mutation thereof.

In some embodiments, a prime editor comprises a Cas9 polypeptide having a mutation in the HNH domain that reduces or abolishes the nuclease activity of the HNH domain. In some embodiments, the Cas9 polypeptide comprises a mutation at amino acid H840 as compared to a wild type SpCas9 as set forth in SEQ ID NO: 592, or a corresponding mutation thereof. In some embodiments, the Cas9 polypeptide comprises a H840A mutation as compared to a wild type SpCas9 as set forth in SEQ ID NO: 592, or a corresponding mutation thereof. In some embodiments, the Cas9 polypeptide comprises a mutation at amino acid E762, D839, H840, N854, N856, N863, H982, H983, A984, D986, and/or a A987 as compared to a wild type SpCas9 as set forth in SEQ ID NO: 592, or a corresponding mutation thereof. In some embodiments, the Cas9 polypeptide comprises a E762A, D839A, H840A, N854A, N856A, N863A, H982A, H983A, A984A, and/or a D986A mutation as compared to a wild type SpCas9 as set forth in SEQ ID NO: 592, or a corresponding mutation thereof. In some embodiments, the Cas9 polypeptide comprises a mutation at amino acid residue R221, N394, and/or H840 as compared to a wild type SpCas9 (e.g., SEQ ID NO: 592). In some embodiments, the Cas9 polypeptide comprises a R221K, N394L, and/or H840A mutation as compared to a wild type SpCas9 as set forth in SEQ ID NO: 592, or a corresponding mutation thereof. In some embodiments, the Cas9 polypeptide comprises a mutation at amino acid residue R220, N393, and/or H839 as compared to a wild type SpCas9 (e.g., SEQ ID NO:592) lacking a N-terminal methionine, or a corresponding mutation thereof. In some embodiments, the Cas9 polypeptide comprises a R220K, N393K, and/or H839A mutation as compared to a wild type SpCas9 (as set forth in SEQ ID NO: 592) lacking a N-terminal methionine, or a corresponding mutation thereof.

In some embodiments, a prime editor comprises a Cas9 having one or more amino acid substitutions in both the HNH domain and the RuvC domain that reduce or abolish the nuclease activity of both the HNH domain and the RuvC domain. In some embodiments, the prime editor comprises a nuclease inactive Cas9, or a nuclease dead Cas9 (dCas9). In some embodiments, the dCas9 comprises a H840X substitution and a DiOX mutation compared to a wild type SpCas9 as set forth in SEQ ID NO: 592 or corresponding mutations thereof, wherein X is any amino acid other than H for the H840X substitution and any amino acid other than D for the D1OX substitution. In some embodiments, the dead Cas9 comprises a H840A and a D1OA mutation as compared to a wild type SpCas9 as set forth in SEQ ID NO: 592, or corresponding mutations thereof.

In some embodiments, the N-terminal methionine is removed from the amino acid sequence of a Cas9 nickase, or from any Cas9 variant, ortholog, or equivalent disclosed or contemplated herein. For example, methionine-minus (Met (-)) Cas9 nickases include any one of the sequences set forth in SEQ ID NOs: 594, 597, 600, 603, 606, 609, 612, 615, 618, or 619, or a variant thereof having an amino acid sequence that has at least 80%, at least 85%, at least 90%, at least 95%, or at least 99% sequence identity thereto.

Besides dead Cas9 and Cas9 nickase variants, the Cas9 proteins used herein may also include other Cas9 variants having at least about 70%, at least about 80%, at least about 90%, at least about 95%, at least about 96%, at least about 97%, at least about 98%, at least about 99%, at least about 99.5%, or at least about 99.9% sequence identity to any reference Cas9 protein, including any wild type Cas9, or mutant Cas9 (e.g., a dead Cas9 or Cas9 nickase), or fragment Cas9, or circular permutant Cas9, or other variant of Cas9 disclosed herein or known in the art. In some embodiments, a Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to a reference Cas9, e.g., a wild type Cas9. In some embodiments, the Cas9 variant comprises a fragment of a reference Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of a reference Cas9, e.g., a wild type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or at least 99.5% of the amino acid length of a corresponding wild type Cas9.

In some embodiments, a Cas9 fragment is a functional fragment that retains one or more Cas9 activities. In some embodiments, the Cas9 fragment is at least 100 amino acids in length. In some embodiments, the fragment is at least 100, 150, 200, 250, 300, 350, 400, 450, 500, 550, 600, 650, 700, 750, 800, 850, 900, 950, 1000, 1050, 1100, 1150, 1200, 1250, or at least 1300 amino acids in length.

In some embodiments, a prime editor comprises a Cas protein, e.g., Cas9, containing modifications that allow altered PAM recognition. In prime editing using a Cas-protein-based prime editor, a “protospacer adjacent motif (PAM)”, PAM sequence, or PAM-like motif, may be used to refer to a short DNA sequence immediately following the protospacer sequence on the PAM strand of the target gene. In some embodiments, the PAM is recognized by the Cas nuclease in the prime editor during prime editing. In certain embodiments, the PAM is required for target binding of the Cas protein. The specific PAM sequence required for Cas protein recognition may depend on the specific type of the Cas protein. A PAM can be 1, 2, 3, 4, 5, 6, 7, 8, 9, 10 or more nucleotides in length. In some embodiments, a PAM is between 2-6 nucleotides in length. In some embodiments, the PAM can be a 5′ PAM (i.e., located upstream of the 5′ end of the protospacer). In other embodiments, the PAM can be a 3′ PAM (i.e., located downstream of the 5′ end of the protospacer).In some embodiments, the Cas protein of a prime editor recognizes a canonical PAM, for example, a SpCas9 recognizes 5′-NGG-3′ PAM. In some embodiments, the Cas protein of a prime editor has altered or non-canonical PAM specificities. Exemplary PAM sequences and corresponding Cas variants are described in Table 2 below. It should be appreciated that for each of the variants provided, the Cas protein comprises one or more of the amino acid substitutions as indicated compared to a wild type Cas protein sequence, for example, the Cas9 as set forth in SEQ ID NO: 592. The PAM motifs as shown in Table 2 below are in the order of 5′ to 3′. In some embodiments, the Cas proteins of the disclosure can also be used to direct transcriptional control of target sequences, for example silencing transcription by sequence-specific binding to target sequences. In some embodiments, a Cas protein described herein may have one or mutations in a PAM recognition motif. In some embodiments, a Cas protein described herein may have altered PAM specificity.

As used in PAM sequences in Table 2, “N” refers to any one of nucleotides A, G, C, or T, “R” refers to nucleotides A or G, “W” refers to A or T, “V” refers to A, C, or G; “Y” refers to nucleotide C or T.

TABLE 2
Cas protein variants and corresponding PAM sequences

| Variant | PAM |
|---|---|
| spCas9 (wild type) | NGG, NGA, NAG, NGNGA |
| spCas9- VRVRFRR | NG |
| (R1335V/L1111R/D1135V/G1218R/E1219F/A1322R/T1337R) | |
| spCas9-VQR (D1135V/R1335Q/T1337R) | NGA |
| spCas9-EQR (D1135E/R1335Q/T1337R) | NGA |
| spCas9-VRER (D1135V/G1218R/R1335E/T1337R) | NGCG |
| spCas9-VRQR (D1135V/G1218R/R1335Q/T1337R) | NGA |
| Cas9-NG (L1111R, D1135V, G1218R, E1219F, A1322R, | NGN |
| T1337R, R1335V) | |
| SpG Cas9 (D1135L, S1136W, G1218K, E1219Q, R1335Q, | NGN |
| T1337R) | |
| SyRY Cas9 | NRN |
| (A61R, L1111R, N1317R, A1322R, and R1333P) | |
| xCas9 (E480K, E543D, E1219V, K294R, Q1256K, A262T, | NGN |
| S409I, M694I) | |
| SluCa9 | NNGG |
| sRGN1, sRGN2, sRGN4, sRGN3.1, sRGN3.3 | NNGG |
| saCas9 | NNGRRT, NNGRRN |
| saCas9-KKH (E782K, N968K, R1015H) | NNNRRT |
| spCas9-MQKSER (D1135M, S1136Q, G1218K, E1219S, | NGCG/NGCN |
| R1335E, T1337R) | |
| spCas9-LRKIQK (D1135L, S1136R, G1218K, E1219I, | NGTN |
| R1335Q, T1337K) | |
| spCas9-LRVSQK (D1135L, S1136R, G1218V, E1219S, | NGTN |
| R1335Q, T1337K) | |
| spCas9-LRVSQL(D1135L, S1136R, G1218V, E1219S, | NGTN |
| R1335Q, T1337L) | |
| Cpf1 | TTTV |
| Spy-Mac | NAA |
| NmCas9 | NNNNGATT |
| StCas9 | NNAGAAW |
| TdCas9 | NAAAAC |

Provided herein in some embodiments are example sequences for PEgRNAs, including PEgRNA spacers, PBS, RTT, and ngRNA spacers for a prime editing system comprising a nuclease that recognizes the PAM sequence “NG.” In some embodiments, a PAM motif on the edit strand comprises an “NG” motif, wherein N is any nucleotide.

In some embodiments, a prime editor comprises a Cas9 polypeptide comprising one or mutations selected from the group consisting of: A61R, LI IR, D 1i35V, R221K, A262T, R324L, N394K, S409I, S409I, E427G, E480K, M495V, N497A, Y515N, K526E, F539S, E543D, R654L, R661A, R661L, R691A, N692A, M694A, M694I, Q695A, H698A, R753G, M763I, K848A, K890N, Q926A, K1003A, R1060A, LIIIIR, RI1i4G, Di135E, Di135L, Di135N, S1136W, V1139A, D1180G, G1218K, G1218R, G1218S, E1219Q, E1219V, E1219V, Q1221H, P1249S, E1253K, N1317R, A1320V, P1321S, A1322R, I1322V, D1332G, R1332N, A1332R, R1333K, R1333P, R1335L, R1335Q, R1335V, T1337N, T1337R, S1338T, H1349R, and any combinations thereof as compared to a wildtype SpCas9 polypeptide as set forth in SEQ ID NO: 592.

In some embodiments, a prime editor comprises a SaCas9 polypeptide. In some embodiments, the SaCas9 polypeptide comprises one or more of mutations E782K, N968K, and RiO15H as compared to a wild type SaCas9. In some embodiments, a prime editor comprises a FnCas9 polypeptide, for example, a wildtype FnCas9 polypeptide or a FnCas9 polypeptide comprising one or more of mutations E1369R, E1449H, or R1556A as compared to the wild type FnCas9. In some embodiments, a prime editor comprises a Sc Cas9, for example, a wild type ScCas9 or a ScCas9 polypeptide comprises one or more of mutations I367K, G368D, I369K, H37iL, T375S, T376G, and T1227K as compared to the wild type ScCas9. In some embodiments, a prime editor comprises a Sti Cas9 polypeptide, a St3 Cas9 polypeptide, or a SluCas9 polypeptide.

In some embodiments, a prime editor comprises a Cas polypeptide that comprises a circular permutant Cas variant. For example, a Cas9 polypeptide of a prime editor may be engineered such that the N-terminus and the C-terminus of a Cas9 protein (e.g., a wild type Cas9 protein, or a Cas9 nickase) are topically rearranged to retain the ability to bind DNA when complexed with a guide RNA (gRNA). An exemplary circular permutant configuration may be N-terminus-[original C-terminus]-[original N-terminus]-C-terminus. Any of the Cas9 proteins described herein, including any variant, ortholog, or naturally occurring Cas9 or equivalent thereof, may be reconfigured as a circular permutant variant.

In various embodiments, the circular permutants of a Cas protein, e.g., a Cas9, may have the following structure: N-terminus-[original C-terminus]-[optional linker]-[original N-terminus]-C-terminus. In some embodiments, a circular permutant Cas9 comprises any one of the following structures (amino acid positions as set forth in SEQ ID NO: 592):
N-terminus-[1268-1368]-[optional linker]-[1-1267]-C-terminus;
N-terminus-[1168-1368]-[optional linker]-[1-1167]-C-terminus;
N-terminus-[1068-1368]-[optional linker]-[1-1067]-C-terminus;
N-terminus-[968-1368]-[optional linker]-[1-967]-C-terminus;
N-terminus-[868-1368]-[optional linker]-[1-867]-C-terminus;
N-terminus-[768-1368]-[optional linker]-[1-767]-C-terminus;
N-terminus-[668-1368]-[optional linker]-[1-667]-C-terminus;
N-terminus-[568-1368]-[optional linker]-[1-567]-C-terminus;
N-terminus-[468-1368]-[optional linker]-[1-467]-C-terminus;
N-terminus-[368-1368]-[optional linker]-[1-367]-C-terminus;
N-terminus-[268-1368]-[optional linker]-[1-267]-C-terminus;
N-terminus-[168-1368]-[optional linker]-[1-167]-C-terminus;
N-terminus-[68-1368]-[optional linker]-[1-67]-C-terminus;
N-terminus-[10-1368]-[optional linker]-[1-9]-C-terminus, orthe corresponding circular permutants of other Cas9 proteins (including other Cas9 orthologs, variants, etc).

In some embodiments, a circular permutant Cas9 comprises any one of the following structures (amino acid positions as set forth in SEQ ID NO: 592-1368 amino acids of UniProtKB—Q99ZW2:
N-terminus-[102-1368]-[optional linker]-[1-101]-C-terminus;
N-terminus-[1028-1368]-[optional linker]-[1-1027]-C-terminus;
N-terminus-[1041-1368]-[optional linker]-[1-1043]-C-terminus;
N-terminus-[1249-1368]-[optional linker]-[1-1248]-C-terminus; or
N-terminus-[1300-1368]-[optional linker]-[1-1299]-C-terminus, or the corresponding circular permutants of other Cas9 proteins (including other Cas9 orthologs, variants, etc).

In some embodiments, a circular permutant Cas9 comprises any one of the following structures (amino acid positions as set forth in SEQ ID NO: 592-1368 amino acids of UniProtKB—Q99ZW2N-terminus-[103-1368]-[optional linker]-[1-102]-C-terminus:
N-terminus-[1029-1368]-[optional linker]-[1-1028]-C-terminus;
N-terminus-[1042-1368]-[optional linker]-[1-1041]-C-terminus;
N-terminus-[1250-1368]-[optional linker]-[1-1249]-C-terminus; or
N-terminus-[1301-1368]-[optional linker]-[1-1300]-C-terminus, or the corresponding circular permutants of other Cas9 proteins (including other Cas9 orthologs, variants, etc).

In some embodiments, the circular permutant can be formed by linking a C-terminal fragment of a Cas9 to an N-terminal fragment of a Cas9, either directly or by using a linker, such as an amino acid linker. In some embodiments, the C-terminal fragment may correspond to the 95% or more of the C-terminal amino acids of a Cas9 (e.g., amino acids about 1300-1368 as set forth in SEQ ID No: 592 or corresponding amino acid positions thereof), or the 90%, 85%, 80%, 75%, 70%, 65%, 60%, 55%, 50%, 45%, 40%, 35%, 30%, 25%, 20%, 15%, 10%, or 5% or more amino acids of the C-terminal of a Cas9 (e.g., Cas9 of SEQ ID NO: 592 or a ortholog or a variant thereof). The N-terminal portion may correspond to 95% or more of the amino acids of the N-terminal of a Cas9 (e.g., amino acids about 1-1300 as set forth in SEQ ID No: 592 or a ortholog or a variant thereof), or 90%, 85%, 80%, 75%, 70%, 65%, 60%, 55%, 50%, 45%, 40%, 35%, 30%, 25%, 20%, 15%, 10%, or 5% or more of the N-terminal amino acids of a Cas9 (e.g., as set forth in SEQ ID No: 592 or corresponding amino acid positions thereof).

In some embodiments, the circular permutant can be formed by linking a C-terminal fragment of a Cas9 to an N-terminal fragment of a Cas9, either directly or by using a linker, such as an amino acid linker. In some embodiments, the C-terminal fragment that is rearranged to the N-terminus includes or corresponds to the C-terminal 30% or less of the amino acids of a Cas9 (e.g., amino acids 1012-1368 as set forth in SEQ ID No: 592 or corresponding amino acid positions thereof). In some embodiments, the C-terminal fragment that is rearranged to the N-terminus, includes or corresponds to the C-terminal 30%, 29%, 28%, 27%, 26%, 25%, 24%, 23%, 22%, 21%, 20%, 19%, 18%, 17%, 16%1, 15%1, 4%, 3%, 12%, 11%, 10%, 9%, 8%, 7%, 6%,5%, 4%, 3%, 2%, or 1% of the amino acids of a Cas9 (e.g., as set forth in SEQ ID No: 592 or corresponding amino acid positions thereof). In some embodiments, the C-terminal fragment that is rearranged to the N-terminus, includes or corresponds to the C-terminal 410 residues or less of a Cas9 (e.g., as set forth in SEQ ID No: 592 or corresponding amino acid positions thereof). In some embodiments, the C-terminal portion that is rearranged to the N-terminus, includes or corresponds to the C-terminal 410, 400, 390, 380, 370, 360, 350, 340, 330, 320, 310,300, 290, 280, 270, 260, 250, 240, 230, 220, 210, 200, 190, 180, 170, 160, 150, 140, 130, 120, 110, 100, 90, 80, 70, 60, 50, 40, 30, 20, or 10 residues of a Cas9 (e.g., as set forth in SEQ ID No: 592 or corresponding amino acid positions thereof). In some embodiments, the C-terminal portion that is rearranged to the N-terminus includes or corresponds to the C-terminal 357, 341, 328, 120, or 69 residues of a Cas9 (e.g., as set forth in SEQ ID No: 592 or corresponding amino acid positions thereof).

In other embodiments, circular permutant Cas9 variants may be a topological rearrangement of a Cas9 primary structure based on the following method, which is based on S. pyogenes Cas9 of SEQ ID NO: 592: (a) selecting a circular permutant (CP) site corresponding to an internal amino acid residue of the Cas9 primary structure, which dissects the original protein into two halves: an N-terminal region and a C-terminal region; (b) modifying the Cas9 protein sequence (e.g., by genetic engineering techniques) by moving the original C-terminal region (comprising the CP site amino acid) to precede the original N-terminal region, thereby forming a new N-terminus of the Cas9 protein that now begins with the CP site amino acid residue. The CP site can be located in any domain of the Cas9 protein, including, for example, the helical-II domain, the RuvCIII domain, or the CTD domain. For example, the CP site may be located (as set forth in SEQ ID No: 592 or corresponding amino acid positions thereof) at original amino acid residue 181, 199, 230, 270, 310, 1010, 1016, 1023, 1029, 1041, 1247, 1249, or 1282. Thus, once relocated to the N-terminus, original amino acid 181, 199, 230, 270, 310, 1010, 1016, 1023, 1029, 1041, 1247, 1249, or 1282 would become the new N-terminal amino acid. Nomenclature of these CP-Cas9 proteins may be referred to as Cas9-CP1⁸¹, Cas9-CP1⁹⁹, Cas9-CP²³⁰, Cas9-CP²⁷⁰, Cas9-CP³¹⁰, Cas9-CP⁰¹⁰, Cas9-CP1⁰¹⁶, Cas9-CP1⁰²³, Cas9-CP1⁰²⁹, Cas9-CP¹⁰⁴¹, Cas9-CP1²⁴⁷, Cas9-CP1²⁴⁹, and Cas9-CP1²⁸², respectively. This description is not meant to be limited to making CP variants from SEQ ID NO: 592, but may be implemented to make CP variants in any Cas9 sequence, either at CP sites that correspond to these positions, or at other CP sites entirely. This description is not meant to limit the specific CP sites in any way. Virtually any CP site may be used to form a CP-Cas9 variant.

In some embodiments, a prime editor comprises a Cas9 functional variant that is of smaller molecular weight than a wild type SpCas9 protein. In some embodiments, a smaller-sized Cas9 functional variant may facilitate delivery to cells, e.g., by an expression vector, nanoparticle, or other means of delivery. In certain embodiments, a smaller-sized Cas9 functional variant is a Class 2 Type II Cas protein. In certain embodiments, a smaller-sized Cas9 functional variant is a Class 2 Type V Cas protein. In certain embodiments, a smaller-sized Cas9 functional variant is a Class 2 Type VI Cas protein.

In some embodiments, a prime editor comprises a SpCas9 that is 1368 amino acids in length and has a predicted molecular weight of 158 kilodaltons. In some embodiments, a prime editor comprises a Cas9 functional variant or functional fragment that is less than 1300 amino acids, less than 1290 amino acids, than less than 1280 amino acids, less than 1270 amino acids, less than 1260 amino acid, less than 1250 amino acids, less than 1240 amino acids, less than 1230 amino acids, less than 1220 amino acids, less than 1210 amino acids, less than 1200 amino acids, less than 1190 amino acids, less than 1180 amino acids, less than 1170 amino acids, less than 1160 amino acids, less than 1150 amino acids, less than 1140 amino acids, less than 1130 amino acids, less than 1120 amino acids, less than 1110 amino acids, less than 1100 amino acids, less than 1050 amino acids, less than 1000 amino acids, less than 950 amino acids, less than 900 amino acids, less than 850 amino acids, less than 800 amino acids, less than 750 amino acids, less than 700 amino acids, less than 650 amino acids, less than 600 amino acids, less than 550 amino acids, or less than 500 amino acids, but at least larger than about 400 amino acids and retaining the one or more functions, e.g., DNA binding function, of the Cas9 protein.

In some embodiments, the Cas protein may include any CRISPR associated protein, including but not limited to, Cas12a, Cas12bl, Cas1, Cas1B, Cas2, Cas3, Cas4, Cas5, Cas6, Cas7, Cas8, Cas9 (also known as Csn1 and Csx12), Cas10, Csyl, Csy2, Csy3, Cse1, Cse2, Csc1, Csc2, Csa5, Csn2, Csm2, Csm3, Csm4, Csm5, Csm6, Cmrl, Cmr3, Cmr4, Cmr5, Cmr6, Csbl, Csb2, Csb3, Csx17, Csx14, Csx10, Csx16, CsaX, Csx3, Csx1, Csx15, Csfl, Csf2, Csf3, Csf4, homologs thereof, or modified versions thereof, and preferably comprising a nickase mutation (e.g., a mutation corresponding to the D10A mutation of the wild type Cas9 polypeptide of SEQ ID NO: 592). In various other embodiments, the napDNAbp can be any of the following proteins: a Cas9, a Cas12a (Cpf1), a Cas12e (CasX), a Cas12d (CasY), a Cas12bl (C2c1), a Cas13a (C2c2), a Cas12c (C2c3), a GeoCas9, a CjCas9, a Cas12g, a Cas12h, a Cas12i, a Cas13b, a Cas13c, a Cas13d, a Cas14, a Csn2, an xCas9, an SpCas9-NG, a circularly permuted Cas9, or an Argonaute (Ago) domain, or a functional variant or fragment thereof.

Exemplary Cas proteins and nomenclature are shown in Table 3 below:

TABLE 3
Exemplary Cas proteins and nomenclature

| | |
|---|---|
| Legacy nomenclature | Current nomenclature |

type II CRISPR-Cas enzymes

| | |
|---|---|
| Cas9 | same |

type V CRISPR-Cas enzymes

| | |
|---|---|
| Cpf1 | Cas12a |
| CasX | Cas12e |
| C2c1 | Cas12b1 |
| Cas12b2 | same |
| C2c3 | Cas12c |
| CasY | Cas12d |
| C2c4 | same |
| C2c8 | same |
| C2c5 | same |
| C2c10 | same |
| C2c9 | same |

type VI CRISPR-Cas enzymes

| | |
|---|---|
| C2c2 | Cas13a |
| Cas13d | same |
| C2c7 | Cas13c |
| C2c6 | Cas13b |

In some embodiments, prime editors described herein may also comprise Cas proteins other than Cas9. For example, in some embodiments, a prime editor as described herein may comprise a Cas12a (Cpf1) polypeptide or functional variants thereof. In some embodiments, the Cas12a polypeptide comprises a mutation that reduces or abolishes the endonuclease domain of the Cas12a polypeptide. In some embodiments, the Cas12a polypeptide is a Cas12a nickase. In some embodiments, the Cas protein comprises an amino acid sequence that comprises at least about 50%, 60%, 70%, 80%, 90%, 91%, 92%, 93%, 94%, 95%, 96%, 97%, 98%, 99%, or 100% sequence identity to a naturally occurring Cas12a polypeptide.

In some embodiments, a prime editor comprises a Cas protein that is a Cas12b (C2cl) or a Cas12c (C2c3) polypeptide. In some embodiments, the Cas protein comprises an amino acid sequence that comprises at least about 50%, 60%, 70%, 80%, 90%, 91%, 92%, 93%, 94%, 95%, 96%, 97%, 98%, 99%, or 100% sequence identity to a naturally occurring Cas12b (C2c1) or Cas12c (C2c3) protein. In some embodiments, the Cas protein is a Cas12b nickase or a Cas12c nickase. In some embodiments, the Cas protein is a Cas12e, a Cas12d, a Cas13, Cas14a, Cas14b, Cas14c, Cas14d, Cas14e, Cas14f, Cas14g, Cas14h, Cas14u, or a Cas(polypeptide. In some embodiments, the Cas protein comprises an amino acid sequence that comprises at least about 50%, 60%, 70%, 80%, 90%, 91%, 92%, 93%, 94%, 95%, 96%, 97%, 98%, 99%, or 100% sequence identity to a naturally-occurring Cas12e, Cas12d, Cas13, Cas14a, Cas14b, Cas14c, Cas14d, Cas14e, Cas14f, Cas14g, Cas14h, Cas14u, or Cas D protein. In some embodiments, the Cas protein is a Cas12e, Cas12d, Cas13, or Cas ϕ nickase.

### Nuclear Localization Sequences

In some embodiments, a prime editor further comprises one or more nuclear localization sequence (NLS). In some embodiments, the NLS helps promote translocation of a protein into the cell nucleus. In some embodiments, a prime editor comprises a fusion protein, e.g., a fusion protein comprising a DNA binding domain and a DNA polymerase, that comprises one or more NLSs. In some embodiments, one or more polypeptides of the prime editor are fused to or linked to one or more NLSs. In some embodiments, the prime editor comprises a DNA binding domain and a DNA polymerase domain that are provided in trans, wherein the DNA binding domain and/or the DNA polymerase domain is fused or linked to one or more NLSs.

In certain embodiments, a prime editor or prime editing complex comprises at least one NLS. In some embodiments, a prime editor or prime editing complex comprises at least two NLSs. In embodiments with at least two NLSs, the NLSs can be the same NLS, or they can be different NLSs.

In some instances, a prime editor may further comprise at least one nuclear localization sequence (NLS). In some cases, a prime editor may further comprise 1 NLS. In some cases, a prime editor may further comprise 2 NLSs. In other cases, a prime editor may further comprise 3 NLSs. In one case, a primer editor may further comprise more than 4, 5, 6, 7, 8, 9 or 10 NLSs.

In addition, the NLSs may be expressed as part of a prime editor complex. In some embodiments, a NLS can be positioned almost anywhere in a protein's amino acid sequence, and generally comprises a short sequence of three or more or four or more amino acids. The location of the NLS fusion can be at the N-terminus, the C-terminus, or positioned anywhere within a sequence of a prime editor or a component thereof (e.g., inserted between the DNA-binding domain and the DNA polymerase domain of a prime editor fusion protein, between the DNA binding domain and a linker sequence, between a DNA polymerase and a linker sequence, between two linker sequences of a prime editor fusion protein or a component thereof, in either N-terminus to C-terminus or C-terminus to N-terminus order). In some embodiments, a prime editor is fusion protein that comprises an NLS at the N terminus. In some embodiments, a prime editor is fusion protein that comprises an NLS at the C terminus. In some embodiments, a prime editor is fusion protein that comprises at least one NLS at both the N terminus and the C terminus. In some embodiments, the prime editor is a fusion protein that comprises two NLSs at the N terminus and/or the C terminus.

Any NLSs that are known in the art are also contemplated herein. The NLSs may be any naturally occurring NLS, or any non-naturally occurring NLS (e.g., an NLS with one or more mutations relative to a wild-type NLS). In some embodiments, the one or more NLSs of a prime editor comprise bipartite NLSs. In some embodiments, a nuclear localization signal (NLS) is predominantly basic. In some embodiments, the one or more NLSs of a prime editor are rich in lysine and arginine residues. In some embodiments, the one or more NLSs of a prime editor comprise proline residues. In some embodiments, a nuclear localization signal (NLS) comprises the sequence

(SEQ ID NO: 626)
MDSLLMNRRKFLYQFKNVRWAKGRRETYLC,
(SEQ ID NO: 637)
KRTADGSEFESPKKKRKV,
(SEQ ID NO: 636)
KRTADGSEFEPKKKRKV,
(SEQ ID NO: 638)
SKRPAAIKKAGQAKKKK,
(SEQ ID NO: 639)
NLSKRPAAIKKAGQAKKKK,
(SEQ ID NO: 640)
RQRRNELKRSF,
or
(SEQ ID NO: 641)
NQSSNFGPMKGGNFGGRSSGPYGGGGQYFAKPRNQGGY

In some embodiments, a NLS is a monopartite NLS. For example, in some embodiments, a NLS is a SV40 large T antigen NLS PKKKRKV (SEQ ID NO: 642). In some embodiments, a NLS is a bipartite NLS. In some embodiments, a bipartite NLS comprises two basic domains separated by a spacer sequence comprising a variable number of amino acids. In some embodiments, a bipartite NLS consists of two basic domains separated by a spacer sequence comprising a variable number of amino acids. In some embodiments, the spacer amino acid sequence comprises the sequence KRXXXXXXXXXXKKKL (Xenopus nucleoplasmin NLS) (SEQ ID NO: 643), wherein X is any amino acid. In some embodiments, the NLS comprises a nucleoplasmin NLS sequence KRPAATKKAGQAKKKK (SEQ ID NO: 644). In some embodiments, a NLS is a noncanonical sequences such as M9 of the hnRNP Al protein, the influenza virus nucleoprotein NLS, or the yeast Gal4 protein NLS. In some embodiments, a bipartite NLS consists of two basic domains separated by a spacer sequence comprising a variable number of amino acids. In some embodiments, a NLS comprises an amino acid sequence that is at least about 50%, 60%, 70%, 80%, 90%, 91%, 92%, 93%, 94%, 95%, 96%, 97%, 98%, 99%, or 100% identical to the amino acid sequence of any one of SEQ ID NOs: 624-644. In some embodiments, a NLS comprises an amino acid sequence selected from the group consisting of 34391-34403. In some embodiments, a prime editing composition comprises a polynucleotide that encodes a NLS that comprises an amino acid sequence that is at least about 50%, 60%, 70%, 80%, 90%, 91%, 92%, 93%, 94%, 95%, 96%, 97%, 98%, 99%, or 100% identical to the amino acid sequence of any one of SEQ ID NOs: 624-644. In some embodiments, a prime editing composition comprises a polynucleotide that encodes a NLS that comprises an amino acid sequence selected from the group consisting of SEQ ID NOs: 624-644.

Any NLSs that are known in the art are also contemplated herein. The NLSs may be any naturally occurring NLS, or any non-naturally occurring NLS (e.g., an NLS with one or more mutations relative to a wild-type NLS). In some embodiments, the one or more NLSs of a prime editor comprise bipartite NLSs. In some embodiments, the one or more NLSs of a prime editor are rich in lysine and arginine residues. In some embodiments, the one or more NLSs of a prime editor comprise proline residues.

Non-limiting examples of NLS sequences are provided in Table 4 below.

TABLE 4
Exemplary nuclear localization sequences

| Description | Sequence |
|---|---|
| NLS of SV40 | PKKKRKV (SEQ ID NO: 624) |
| Large T-AG | |
| NLS | MKRTADGSEFESPKKKRKV (SEQ ID NO: 625) |
| NLS | MDSLLMNRRKFLYQFKNVRWAKGRRETYLC (SEQ ID NO: |
| | 626) |
| NLS of | AVKRPAATKKAGQAKKKKLD (SEQ ID NO: 627) |
| Nucleoplasmin | |
| NLS of EGL-13 | MSRRRKANPTKLSENAKKLAKEVEN (SEQ ID NO: 628) |
| NLS of C-Myc | PAAKRVKLD (SEQ ID NO: 629) |
| NLS of Tus-protein | KLKIKRPVK (SEQ ID NO: 630) |
| NLS of polyoma | VSRKRPRP (SEQ ID NO: 631) |
| large T-AG | |
| NLS of Hepatitis D | EGAPPAKRAR (SEQ ID NO: 633) |
| virus antigen | |
| NLS of Rev protein | RQARRNRRRRWRERNR (SEQ ID NO: 632) |
| NLS of murine p53 | PPQPKKKPLDGE (SEQ ID NO: 634) |
| C terminal linker | SGGSKRTADGSEFEPKKKRKV (SEQ ID NO: 635) |
| and NLS of PE1 | |
| and PE2an | |
| exemplary prime | |
| editor fusion | |
| protein | |
| C Terminal NLS of | KRTADGSEFEPKKKRKV (SEQ ID NO: 636) |
| an exemplary | |
| prime editor fusion | |
| protein | |

### Additional Prime Editor Components

A prime editor described herein may comprise additional functional domains, for example, one or more domains that modify the folding, solubility, or charge of the prime editor. In some instances, the prime editor may comprise a solubility-enhancement (SET) domain.

In some embodiments, a split intein comprises two halves of an intein protein, which may be referred to as a N-terminal half of an intein, or intein-N, and a C-terminal half of an intein, or intein-C, respectively. In some embodiments, the intein-N and the intein-C may each be fused to a protein domain (the N-terminal and the C-terminal exteins). The exteins can be any protein or polypeptides, for example, any prime editor polypeptide component. In some embodiments, the intein-N and intein-C of a split intein can associate non-covalently to form an active intein and catalyze a-trans splicing reaction. In some embodiments, the trans splicing reaction excises the two intein sequences and links the two extein sequences with a peptide bond. As a result, the intein-N and the intein-C are spliced out, and a protein domain linked to the intein-N is fused to a protein domain linked to the intein-C. essentially in same way as a contiguous intein does. In some embodiments, a split-intein is derived from a eukaryotic intein, a bacterial intein, or an archaeal intein. Preferably, the split intein so-derived will possess only the amino acid sequences essential for catalyzing trans-splicing reactions. In some embodiments, an intein-N or an intein-C further comprise one or more amino acid substitutions as compared to a wild type intein-N or wild type intein-C, for example, amino acid substitutions that enhances the trans-splicing activity of the split intein. In some embodiments, the intein-C comprises 4 to 7 contiguous amino acid residues, wherein at least 4 amino acids of which are from the last a-strand of the intein from which it was derived. In some embodiments, the split intein is derived from a Ssp DnaE intein, e.g., Synechocytis sp. PCC6803, or any intein or split intein known in the art, or any functional variants or fragments thereof.

In some embodiments, a prime editor comprises one or more epitope tags. Non-limiting examples of epitope tags include histidine (His) tags, V5 tags, FLAG tags, influenza hemagglutinin (HA) tags, Myc tags, VSV-G tags, thioredoxin (Trx) tags, biotin carboxylase carrier protein (BCCP) tags, myc-tags, calmodulin-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein comprises one or more His tags.

In some embodiments, a prime editor comprises one or more polypeptide domains encoded by one or more reporter genes. Examples of reporter genes include, but are not limited to, glutathione-5-transferase (GST), horseradish peroxidase (HRP), chloramphenicol acetyltransferase (CAT), beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP).

In some embodiments, a prime editor comprises one or more polypeptide domains that binds DNA molecules or binds other cellular molecules. Examples of binding proteins or domains include, but are not limited to, maltose binding protein (MBP), S-tag, Lex A DNA binding domain (DBD) fusions, GAL4 DNA binding domain fusions, and herpes simplex virus (HSV) BP16 protein fusions.

In some embodiments, a prime editor comprises a protein domain that is capable of modifying the intracellular half-life of the prime editor.

In some embodiments, a prime editing complex comprises a fusion protein comprising a DNA binding domain (e.g., Cas9(H840A)) and a reverse transcriptase (e.g., a variant MMLV RT) having the following structure: [NLS]-[Cas9(H840A)]-[linker]-[MMLV_RT(D200N)(T330P)(L603W)(T306K)(W313F)], and a desired PEgRNA. In some embodiments, the prime editing complex comprises a prime editor fusion protein that has the amino acid sequence of SEQ ID NO: 620. In some embodiments, the prime editing complex comprises a prime editor fusion protein that has the amino acid sequence of SEQ ID NO: 621. Sequence of an exemplary prime editor fusion protein comprising a DNA binding domain (e.g., Cas9(H840A)) and a reverse transcriptase (e.g., a variant MMLV RT) having the following structure: [NLS]-[Cas9(H840A)]-[linker]-[MMLV_RT(D200N)(T330P)(L603W)(T306K)(W313F)] and its components are shown in Table 5.

In some embodiments, a prime editing complex comprises a fusion protein comprising a DNA binding domain (e.g., Cas9((R221K N394K H840A)) and a reverse transcriptase (e.g., a variant MMLV RT) having the following structure: [NLS]-[Cas9((R221K N394K H840A)]-[linker]-[MMLV_RT(D200N)(T330P)(L603W)(T306K)(W313F)], and a desired PEgRNA. In some embodiments, the prime editing complex comprises a prime editor fusion protein that has the amino acid sequence of SEQ ID NO: 622. In some embodiments, the prime editing complex comprises a prime editor fusion protein that has the amino acid sequence of SEQ ID NO: 623. Sequence of an exemplary prime editor fusion protein comprising a DNA binding domain (e.g., Cas9(H840A)) and a reverse transcriptase (e.g., a variant MMLV RT) having the following structure: [NLS]-[Cas9 (R221K N394K H840A)]-[linker]-[MMLV_RT(D200N)(T330P)(L603W)(T306K)(W313F)] and its components are shown in Table 6.

Polypeptides comprising components of a prime editor may be fused via peptide linkers, or may be provided in trans relevant to each other. For example, a reverse transcriptase may be expressed, delivered, or otherwise provided as an individual component rather than as a part of a fusion protein with the DNA binding domain. In such cases, components of the prime editor may be associated through non-peptide linkages or co-localization functions. In some embodiments, a prime editor further comprises additional components capable of interacting with, associating with, or capable of recruiting other components of the prime editor or the prime editing system. For example, a prime editor may comprise an RNA-protein recruitment polypeptide that can associate with an RNA-protein recruitment RNA aptamer. In some embodiments, an RNA-protein recruitment polypeptide can recruit, or be recruited by, a specific RNA sequence. Non limiting examples of RNA-protein recruitment polypeptide and RNA aptamer pairs include a MS2 coat protein and a MS2 RNA hairpin, a PCP polypeptide and a PP7 RNA hairpin, a Com polypeptide and a Com RNA hairpin, a Ku protein, and a telomerase Ku binding RNA motif, and a Sm7 protein and a telomerase Sm7 binding RNA motif. In some embodiments, the prime editor comprises a DNA binding domain fused or linked to an RNA-protein recruitment polypeptide. In some embodiments, the prime editor comprises a DNA polymerase domain fused or linked to an RNA-protein recruitment polypeptide. In some embodiments, the DNA binding domain and the DNA polymerase domain fused to the RNA-protein recruitment polypeptide, or the DNA binding domain fused to the RNA-protein recruitment polypeptide and the DNA polymerase domain are co-localized by the corresponding RNA-protein recruitment RNA aptamer of the RNA-protein recruitment polypeptide. In some embodiments, the corresponding RNA-protein recruitment RNA aptamer fused or linked to a portion of the PEgRNA or ngRNA. For example, an MS2 coat protein fused or linked to the DNA polymerase and a MS2 hairpin installed on the PEgRNA for co-localization of the DNA polymerase and the RNA-guided DNA binding domain (e.g., a Cas9 nickase).

In some embodiments, a prime editor comprises a polypeptide domain, an MS2 coat protein (MCP), that recognizes an MS2 hairpin. In some embodiments, the nucleotide sequence of the MS2 hairpin (or equivalently referred to as the “MS2 aptamer”) is: GCCAACATGAGGATCACCCATGTCTGCAGGGCC (SEQ ID NO: 645). In some embodiments, the amino acid sequence of the MCP is: GSASNFTQFVLVDNGGTGDVTVAPSNFANGVAEWISSNSRSQAYKVTCSVRQSSAQNRKYTI KVEVPKVATQTVGGEELPVAGWRSYLNMELTIPIFATNSDCELIVKAMQGLLKDGNPIPSAIA ANSGIY (SEQ ID NO: 646). In certain embodiments, components of a prime editor are directly fused to each other. In certain embodiments, components of a prime editor are associated to each other via a linker.

As used herein, a linker can be any chemical group or a molecule linking two molecules or moieties, e.g., a DNA binding domain and a polymerase domain of a prime editor. In some embodiments, a linker is an organic molecule, group, polymer, or chemical moiety. In some embodiments, the linker comprises a non-peptide moiety. The linker may be as simple as a covalent bond, or it may be a polymeric linker many atoms in length, for example, a polynucleotide sequence. In certain embodiments, the linker is a covalent bond (e.g., a carbon-carbon bond, disulfide bond, carbon-heteroatom bond, etc.).

In certain embodiments, two or more components of a prime editor are linked to each other by a peptide linker. In some embodiments, a peptide linker is 5-100 amino acids in length, for example, 2,3,4,5,6,7, 8,9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. In some embodiments, the peptide linker is 16 amino acids in length, 24 amino acids in length, 64 amino acids in length, or 96 amino acids in length.

In some embodiments, the linker comprises the amino acid sequence (GGGGS)n (SEQ ID NO: 647), (G)n (SEQ ID NO: 648), (EAAAK)n (SEQ ID NO: 649), (GGS)n (SEQ ID NO: 650), (SGGS)n (SEQ ID NO: 651), (XP)n (SEQ ID NO: 652), or any combination thereof, wherein n is independently an integer between 1 and 30, and wherein X is any amino acid. In some embodiments, the linker comprises the amino acid sequence (GGS)n (SEQ ID NO: 653), wherein n is 1, 3, or 7. In some embodiments, the linker comprises the amino acid sequence SGSETPGTSESATPES (SEQ ID NO: 654). In some embodiments, the linker comprises the amino acid sequence SGGSSGGSSGSETPGTSESATPESSGGSSGGS (SEQ ID NO: 655). In some embodiments, the linker comprises the amino acid sequence SGGSGGSGGS (SEQ ID NO: 656). In some embodiments, the linker comprises the amino acid sequence SGGS (SEQ ID NO: 657). In other embodiments, the linker comprises the amino acid sequence SGGSSGGSSGSETPGTSESATPESAGSYPYDVPDYAGSAAPAAKKKKLDGSGSGGSSGGS (SEQ ID NO: 658). In some embodiments, a linker comprises 1-100 amino acids. In some embodiments, the linker comprises the amino acid sequence SGSETPGTSESATPES (SEQ ID NO: 654). In some embodiments, the linker comprises the amino acid sequence SGGSSGGSSGSETPGTSESATPESSGGSSGGS (SEQ ID NO: 655). In some embodiments, the linker comprises the amino acid sequence SGGSGGSGGS (SEQ ID NO: 656). In some embodiments, the linker comprises the amino acid sequence SGGS (SEQ ID NO: 657). In some embodiments, the linker comprises the amino acid sequence GGSGGS (SEQ ID NO: 659), GGSGGSGGS (SEQ ID NO: 660), SGGSSGGSSGSETPGTSESATPESAGSYPYDVPDYAGSAAPAAKKKKLDGSGSGGSSGGS (SEQ ID NO: 658), or SGGSSGGSSGSETPGTSESATPESSGGSSGGSS (SEQ ID NO: 661). In some embodiments, the linker comprises the amino acid sequence SGGSSGGSSGSETPGTSESATPESSGGSSGGSS (SEQ ID NO: 661).

(SEQ ID NO: 661)
SGGSSGGSSGSETPGTSESATPESSGGSSGGSS.

In certain embodiments, two or more components of a prime editor are linked to each other by a non-peptide linker. In some embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

Components of a prime editor may be connected to each other in any order. In some embodiments, the DNA binding domain and the DNA polymerase domain of a prime editor may be fused to form a fusion protein, or may be joined by a peptide or protein linker, in any order from the N terminus to the C terminus. In some embodiments, a prime editor comprises a DNA binding domain fused or linked to the C-terminal end of a DNA polymerase domain. In some embodiments, a prime editor comprises a DNA binding domain fused or linked to the N-terminal end of a DNA polymerase domain. In some embodiments, the prime editor comprises a fusion protein comprising the structure NH2-[DNA binding domain]-[polymerase]-COOH; or NH2-[polymerase]-[DNA binding domain]-COOH, wherein each instance of” ]-[” indicates the presence of an optional linker sequence. In some embodiments, a prime editor comprises a fusion protein and a DNA polymerase domain provided in trans, wherein the fusion protein comprises the structure NH2-[DNA binding domain]-[RNA-protein recruitment polypeptide]-COOH. In some embodiments, a prime editor comprises a fusion protein and a DNA binding domain provided in trans, wherein the fusion protein comprises the structure NH2-[DNA polymerase domain]-[RNA-protein recruitment polypeptide]-COOH.

In some embodiments, a prime editor fusion protein, a polypeptide component of a prime editor, or a polynucleotide encoding the prime editor fusion protein or polypeptide component, may be split into an N-terminal half and a C-terminal half or polypeptides that encode the N-terminal half and the C terminal half, and provided to a target DNA in a cell separately. For example, in certain embodiments, a prime editor fusion protein may be split into a N-terminal and a C-terminal half for separate delivery in AAV vectors, and subsequently translated and colocalized in a target cell to reform the complete polypeptide or prime editor protein. In such cases, separate halves of a protein or a fusion protein may each comprise a split-intein to facilitate colocalization and reformation of the complete protein or fusion protein by the mechanism of intein facilitated trans splicing. In some embodiments, a prime editor comprises a N-terminal half fused to an intein-N, and a C-terminal half fused to an intein-C, or polynucleotides or vectors (e.g., AAV vectors) encoding each thereof. When delivered and/or expressed in a target cell, the intein-N and the intein-C can be excised via protein trans-splicing, resulting in a complete prime editor fusion protein in the target cell. In some embodiments, an exemplary protein described herein may lack a methionine residue at the N-terminus.

In some embodiments, a prime editor fusion protein comprises a Cas9(H840A) nickase and a wild type M-MLV RT. In some embodiments, a prime editor fusion protein comprises a Cas9(H840A) nickase and a M-MLV RT that comprises amino acid substitutions D200N, T330P, T306K, W313F, and L603W compared to a wild type M-MLV RT.

In some embodiments, a prime editor fusion protein comprises a Cas9(H840A) nickase and a M-MLV RT that comprises amino acid substitutions D200N, T330P, T306K, W313F, and L603W compared to a wild type M-MLV RT. The amino acid sequence of an exemplary prime editor fusion protein and its individual components is shown in Table 5.

In some embodiments, a prime editor fusion protein comprises a Cas9 (R221K N394K H840A) nickase and a M-MLV RT that comprises amino acid substitutions D200N, T330P, T306K, W313F, and L603W compared to a wild type M-MLV RT. The amino acid sequence of an exemplary Prime editor fusion protein and its individual components is shown in Table 6.

In some embodiments, an exemplary prime editor protein may comprise an amino acid sequence as set forth in any of the SEQ ID NOs: 620, 621, 622, or 623.

In various embodiments, a prime editor fusion protein comprises an amino acid sequence that is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any one of the exemplary prime editor fusion proteins provided herein, or any of the prime editor fusion sequences described herein or known in the art.

TABLE 5
lists exemplary prime editor fusion proteins and its individual components.

| SEQ | | |
|---|---|---|
| ID | | |
| NO. | DESCRIPTION | SEQUENCE |
| 620 | Exemplary Prime | MKRTADGSEFESPKKKRKVDKKYSIGLDIGTNSVGWAVITDEYKVPSKKFKVLGNTD |
| | Editor [NLS]- | RHSIKKNLIGALLFDSGETAEATRLKRTARRRYTRRKNRICYLQEIFSNEMAKVDDSF |
| | [Cas9(H840A)]- | FHRLEESFLVEEDKKHERHPIFGNIVDEVAYHEKYPTIYHLRKKLVDSTDKADLRLIY |
| | [linker]- | LALAHMIKFRGHFLIEGDLNPDNSDVDKLFIQLVQTYNQLFEENPINASGVDAKAILS |
| | [MMLV_RT(D200N) | ARLSKSRRLENLIAQLPGEKKNGLFGNLIALSLGLTPNFKSNFDLAEDAKLQLSKDTY |
| | (T330P)(L603W) | DDDLDNLLAQIGDQYADLFLAAKNLSDAILLSDILRVNTEITKAPLSASMIKRYDEHH |
| | (T306K)(W313F)]- | QDLTLLKALVRQQLPEKYKEIFFDQSKNGYAGYIDGGASQEEFYKFIKPILEKMDGT |
| | [NLS] | EELLVKLNREDLLRKQRTFDNGSIPHQIHLGELHAILRRQEDFYPFLKDNREKIEKIL |
| | | TFRIPYYVGPLARGNSRFAWMTRKSEETITPWNFEEVVDKGASAQSFIERMTNFDKN |
| | | LPNEKVLPKHSLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKDFLDNEENEDI |
| | | LEDIVLTLTLFEDREMIEERLKTYAHLFDDKVMKQLKRRRYTGWGRLSRKLINGIR |
| | | DKQSGKTILDFLKSDGFANRNFMQLIHDDSLTFKEDIQKAQVSGQGDSLHEHIANLA |
| | | GSPAIKKGILQTVKVVDELVKVMGRHKPENIVIEMARENQTTQKGQKNSRERMKRI |
| | | EEGIKELGSQILKEHPVENTQLQNEKLYLYYLQNGRDMYVDQELDINRLSDYDVDAI |
| | | VPQSFLKDDSIDNKVLTRSDKNRGKSDNVPSEEVVKKMKNYWRQLLNAKLITQRKF |
| | | DNLTKAERGGLSELDKAGFIKRQLVETRQITKHVAQILDSRMNTKYDENDKLIREVK |
| | | VITLKSKLVSDFRKDFQFYKVREINNYHHAHDAYLNAVVGTALIKKYPKLESEFVYG |
| | | DYKVYDVRKMIAKSEQEIGKATAKYFFYSNIMNFFKTEITLANGEIRKRPLIETNGET |
| | | GEIVWDKGRDFATVRKVLSMPQVNIVKKTEVQTGGFSKESILPKRNSDKLIARKKD |
| | | WDPKKYGGFDSPTVAYSVLVVAKVEKGKSKKLKSVKELLGITIMERSSFEKNPIDFL |
| | | EAKGYKEVKKDLIIKLPKYSLFELENGRKRMLASAGELQKGNELALPSKYVNFLYLA |
| | | SHYEKLKGSPEDNEQKQLFVEQHKHYLDEIIEQISEFSKRVILADANLDKVLSAYNKH |
| | | RDKPIREQAENIIHLFTLTNLGAPAAFKYFDTTIDRKRYTSTKEVLDATLIHQSITGLY |
| | | ETRIDLSQLGGDSGGSSGGSSGSETPGTSESATPESSGGSSGGSSTLNIEDEYRLHETSKEP |
| | | DVSLGSTWLSDFPQAWAETGGMGLAVRQAPLIIPLKATSTPVSIKQYPMSQEARLGIKPHIQRLL |
| | | DQGILVPCQSPWNTPLLPVKKPGTNDYRPVQDLREVNKRVEDIHPTVPNPYNLLSGLPPSHQW |
| | | YTVLDLKDAFFCLRLHPTSQPLFAFEWRDPEMGISGQLTWTRLPQGFKNSPTLFNEALHRDLA |
| | | DFRIQHPDLILLQYVDDLLLAATSELDCQQGTRALLQTLGNLGYRASAKKAQICQKQVKYLGYL |
| | | LKEGQRWLTEARKETVMGQPTPKTPRQLREFLGKAGFCRLFIPGFAEMAAPLYPLTKPGTLFN |
| | | WGPDQQKAYQEIKQALLTAPALGLPDLTKPFELFVDEKQGYAKGVLTQKLGPWRRPVAYLSK |
| | | KLDPVAAGWPPCLRMVAAIAVLTKDAGKLTMGQPLVILAPHAVEALVKQPPDRWLSNARMTHY |
| | | QALLLDTDRVQFGPVVALNPATLLPLPEEGLQHNCLDILAEAHGTRPDLTDQPLPDADHTWYT |
| | | DGSSLLQEGQRKAGAAVTTETEVIWAKALPAGTSAQRAELIALTQALKMAEGKKLNVYTDSRYA |
| | | FATAHIHGEIYRRRGWLTSEGKEIKNKDEILALLKALFLPKRLSIIHCPGHQKGHSAEARGNRMA |
| | | DQAARKAAITETPDTSTLLIENSSPSGGSKRTADGSEFEPKKKRKV |
| | | KEY: |
| | | NUCLEAR LOCALIZATION SEQUENCE (NLS) |
| | | CAS9(H840A) |
| | | 33-AMINO ACID LINKER |
| | | M-MLV REVERSE TRANSCRIPTASE |
| 621 | Exemplary Prime | KRTADGSEFESPKKKRKVDKKYSIGLDIGTNSVGWAVITDEYKVPSKKFKVLGNTDRHSI |
| | Editor [NLS]- | KKNLIGALLFDSGETAEATRLKRTARRRYTRRKNRICYLQEIFSNEMAKVDDSFFHRLEES |
| | [Cas9(H840A)]- | FLVEEDKKHERHPIFGNIVDEVAYHEKYPTIYHLRKKLVDSTDKADLRLIYLALAHMIKFR |
| | [linker]- | GHFLIEGDLNPDNSDVDKLFIQLVQTYNQLFEENPINASGVDAKAILSARLSKSRRLENLIA |
| | [MMLV_RT(D200N) | QLPGEKKNGLFGNLIALSLGLTPNFKSNFDLAEDAKLQLSKDTYDDDLDNLLAQIGDQYA |
| | (T330P)(L603W) | DLFLAAKNLSDAILLSDILRVNTEITKAPLSASMIKRYDEHHQDLTLLKALVRQQLPEKYK |
| | (T306K)(W313F)]- | EIFFDQSKNGYAGYIDGGASQEEFYKFIKPILEKMDGTEELLVKLNREDLLRKQRTFDNGSI |
| | [NLS] | PHQIHLGELHAILRRQEDFYPFLKDNREKIEKILTFRIPYYVGPLARGNSRFAWMTRKSEET |
| | without N- | ITPWNFEEVVDKGASAQSFIERMTNFDKNLPNEKVLPKHSLLYEYFTVYNELTKVKYVTE |
| | terminal | GMRKPAFLSGEQKKAIVDLLFKTNRKVTVKQLKEDYFKKIECFDSVEISGVEDRFNASLG |
| | methionine | TYHDLLKIIKDKDFLDNEENEDILEDIVLTLTLFEDREMIEERLKTYAHLFDDKVMKQLKR |
| | | RRYTGWGRLSRKLINGIRDKQSGKTILDFLKSDGFANRNFMQLIHDDSLTFKEDIQKAQVS |
| | | GQGDSLHEHIANLAGSPAIKKGILQTVKVVDELVKVMGRHKPENIVIEMARENQTTQKGQ |
| | | KNSRERMKRIEEGIKELGSQILKEHPVENTQLQNEKLYLYYLQNGRDMYVDQELDINRLS |
| | | DYDVDAIVPQSFLKDDSIDNKVLTRSDKNRGKSDNVPSEEVVKKMKNYWRQLLNAKLIT |
| | | QRKFDNLTKAERGGLSELDKAGFIKRQLVETRQITKHVAQILDSRMNTKYDENDKLIREV |
| | | KVITLKSKLVSDFRKDFQFYKVREINNYHHAHDAYLNAVVGTALIKKYPKLESEFVYGDY |
| | | KVYDVRKMIAKSEQEIGKATAKYFFYSNIMNFFKTEITLANGEIRKRPLIETNGETGEIVWD |
| | | KGRDFATVRKVLSMPQVNIVKKTEVQTGGFSKESILPKRNSDKLIARKKDWDPKKYGGF |
| | | DSPTVAYSVLVVAKVEKGKSKKLKSVKELLGITIMERSSFEKNPIDFLEAKGYKEVKKDLII |
| | | KLPKYSLFELENGRKRMLASAGELQKGNELALPSKYVNFLYLASHYEKLKGSPEDNEQK |
| | | QLFVEQHKHYLDEIIEQISEFSKRVILADANLDKVLSAYNKHRDKPIREQAENIIHLFTLTNL |
| | | GAPAAFKYFDTTIDRKRYTSTKEVLDATLIHQSITGLYETRIDLSQLGGDSGGSSGGSSGSE |
| | | TPGTSESATPESSGGSSGGSSTLNIEDEYRLHETSKEPDVSLGSTWLSDFPQAWAETGGMG |
| | | LAVRQAPLIIPLKATSTPVSIKQYPMSQEARLGIKPHIQRLLDQGILVPCQSPWNTPLLPVK |
| | | KPGTNDYRPVQDLREVNKRVEDIHPTVPNPYNLLSGLPPSHQWYTVLDLKDAFFCLRLHP |
| | | TSQPLFAFEWRDPEMGISGQLTWTRLPQGFKNSPTLFNEALHRDLADFRIQHPDLILLQYV |
| | | DDLLLAATSELDCQQGTRALLQTLGNLGYRASAKKAQICQKQVKYLGYLLKEGQRWLTE |
| | | ARKETVMGQPTPKTPRQLREFLGKAGFCRLFIPGFAEMAAPLYPLTKPGTLFNWGPDQQK |
| | | AYQEIKQALLTAPALGLPDLTKPFELFVDEKQGYAKGVLTQKLGPWRRPVAYLSKKLDP |
| | | VAAGWPPCLRMVAAIAVLTKDAGKLTMGQPLVILAPHAVEALVKQPPDRWLSNARMTH |
| | | YQALLLDTDRVQFGPVVALNPATLLPLPEEGLQHNCLDILAEAHGTRPDLTDQPLPDADH |
| | | TWYTDGSSLLQEGQRKAGAAVTTETEVIWAKALPAGTSAQRAELIALTQALKMAEGKKL |
| | | NVYTDSRYAFATAHIHGEIYRRRGWLTSEGKEIKNKDEILALLKALFLPKRLSIIHCPGHQK |
| | | GHSAEARGNRMADQAARKAAITETPDTSTLLIENSSPSGGSKRTADGSEFEPKKKRKV |
| 625 | N-terminal NLS | MKRTADGSEFESPKKKRKV |
| 594 | SpCas9 nickase | DKKYSIGLDIGTNSVGWAVITDEYKVPSKKFKVLGNTDRHSIKKNLIGALLFDSGETAEAT |
| | (H840A) (Met | RLKRTARRRYTRRKNRICYLQEIFSNEMAKVDDSFFHRLEESFLVEEDKKHERHPIFGNIV |
| | Minus) | DEVAYHEKYPTIYHLRKKLVDSTDKADLRLIYLALAHMIKFRGHFLIEGDLNPDNSDVDK |
| | | LFIQLVQTYNQLFEENPINASGVDAKAILSARLSKSRRLENLIAQLPGEKKNGLFGNLIALS |
| | | LGLTPNFKSNFDLAEDAKLQLSKDTYDDDLDNLLAQIGDQYADLFLAAKNLSDAILLSDIL |
| | | RVNTEITKAPLSASMIKRYDEHHQDLTLLKALVRQQLPEKYKEIFFDQSKNGYAGYIDGG |
| | | ASQEEFYKFIKPILEKMDGTEELLVKLNREDLLRKQRTFDNGSIPHQIHLGELHAILRRQED |
| | | FYPFLKDNREKIEKILTFRIPYYVGPLARGNSRFAWMTRKSEETITPWNFEEVVDKGASAQ |
| | | SFIERMTNFDKNLPNEKVLPKHSLLYEYFTVYNELTKVKYVTEGMRKPAFLSGEQKKAIV |
| | | DLLFKTNRKVTVKQLKEDYFKKIECFDSVEISGVEDRFNASLGTYHDLLKIIKDKDFLDNE |
| | | ENEDILEDIVLTLTLFEDREMIEERLKTYAHLFDDKVMKQLKRRRYTGWGRLSRKLINGIR |
| | | DKQSGKTILDFLKSDGFANRNFMQLIHDDSLTFKEDIQKAQVSGQGDSLHEHIANLAGSPA |
| | | IKKGILQTVKVVDELVKVMGRHKPENIVIEMARENQTTQKGQKNSRERMKRIEEGIKELG |
| | | SQILKEHPVENTQLQNEKLYLYYLQNGRDMYVDQELDINRLSDYDVDAIVPQSFLKDDSI |
| | | DNKVLTRSDKNRGKSDNVPSEEVVKKMKNYWRQLLNAKLITQRKFDNLTKAERGGLSE |
| | | LDKAGFIKRQLVETRQITKHVAQILDSRMNTKYDENDKLIREVKVITLKSKLVSDFRKDFQ |
| | | FYKVREINNYHHAHDAYLNAVVGTALIKKYPKLESEFVYGDYKVYDVRKMIAKSEQEIG |
| | | KATAKYFFYSNIMNFFKTEITLANGEIRKRPLIETNGETGEIVWDKGRDFATVRKVLSMPQ |
| | | VNIVKKTEVQTGGFSKESILPKRNSDKLIARKKDWDPKKYGGFDSPTVAYSVLVVAKVEK |
| | | GKSKKLKSVKELLGITIMERSSFEKNPIDFLEAKGYKEVKKDLIIKLPKYSLFELENGRKRM |
| | | LASAGELQKGNELALPSKYVNFLYLASHYEKLKGSPEDNEQKQLFVEQHKHYLDEIIEQIS |
| | | EFSKRVILADANLDKVLSAYNKHRDKPIREQAENIIHLFTLTNLGAPAAFKYFDTTIDRKR |
| | | YTSTKEVLDATLIHQSITGLYETRIDLSQLGGD |
| 661 | Linker between | SGGSSGGSSGSETPGTSESATPESSGGSSGGSS |
| | CAS9 domain | |
| | and RT domain | |
| | (33 amino acids) | |
| 591 | MMLV_RT | TLNIEDEYRLHETSKEPDVSLGSTWLSDFPQAWAETGGMGLAVRQAPLIIPLKATSTPVSIK |
| | D200N T330P | QYPMSQEARLGIKPHIQRLLDQGILVPCQSPWNTPLLPVKKPGTNDYRPVQDLREVNKRV |
| | L603W T306K | EDIHPTVPNPYNLLSGLPPSHQWYTVLDLKDAFFCLRLHPTSQPLFAFEWRDPEMGISGQL |
| | W313F | TWTRLPQGFKNSPTLFNEALHRDLADFRIQHPDLILLQYVDDLLLAATSELDCQQGTRALL |
| | | QTLGNLGYRASAKKAQICQKQVKYLGYLLKEGQRWLTEARKETVMGQPTPKTPRQLREF |
| | | LGKAGFCRLFIPGFAEMAAPLYPLTKPGTLFNWGPDQQKAYQEIKQALLTAPALGLPDLT |
| | | KPFELFVDEKQGYAKGVLTQKLGPWRRPVAYLSKKLDPVAAGWPPCLRMVAAIAVLTK |
| | | DAGKLTMGQPLVILAPHAVEALVKQPPDRWLSNARMTHYQALLLDTDRVQFGPVVALN |
| | | PATLLPLPEEGLQHNCLDILAEAHGTRPDLTDQPLPDADHTWYTDGSSLLQEGQRKAGAA |
| | | VTTETEVIWAKALPAGTSAQRAELIALTQALKMAEGKKLNVYTDSRYAFATAHIHGEIYR |
| | | RRGWLTSEGKEIKNKDEILALLKALFLPKRLSIIHCPGHQKGHSAEARGNRMADQAARKA |
| | | AITETPDTSTLLIENSSP |
| 635 | C- terminal | SGGSKRTADGSEFEPKKKRKV |
| | Linker and NLS | |
| 636 | C-terminal NLS | KRTADGSEFEPKKKRKV |

TABLE 6
Amino acid sequences of an exemplary PE fusion protein and its individual

| SEQ ID | | |
|---|---|---|
| NO. | DESCRIPTION | SEQUENCE |
| 622 | Exemplary prime editor | MKRTADGSEFESPKKKRKVDKKYSIGLDIGTNSVGWAVITDEYKVPSKKF |
| | [NLS]- [Cas9((R220K) | KVLGNTDRHSIKKNLIGALLFDSGETAEATRLKRTARRRYTRRKNRICYL |
| | (R393K)(H839A)]-[linker]- | QEIFSNEMAKVDDSFFHRLEESFLVEEDKKHERHPIFGNIVDEVAYHEKY |
| | [MMLV_RT(D200N)(T330P) | PTIYHLRKKLVDSTDKADLRLIYLALAHMIKFRGHFLIEGDLNPDNSDVD |
| | (L603W)(T306K)(W313F)]- | KLFIQLVQTYNQLFEENPINASGVDAKAILSARLSKSRKLENLIAQLPGEK |
| | [NLS] | KNGLFGNLIALSLGLTPNFKSNFDLAEDAKLQLSKDTYDDDLDNLLAQIG |
| | | DQYADLFLAAKNLSDAILLSDILRVNTEITKAPLSASMIKRYDEHHQDLTL |
| | | LKALVRQQLPEKYKEIFFDQSKNGYAGYIDGGASQEEFYKFIKPILEKMD |
| | | GTEELLVKLKREDLLRKQRTFDNGSIPHQIHLGELHAILRRQEDFYPFLK |
| | | DNREKIEKILTFRIPYYVGPLARGNSRFAWMTRKSEETITPWNFEEVVDK |
| | | GASAQSFIERMTNFDKNLPNEKVLPKHSLLYEYFTVYNELTKVKYVTEG |
| | | MRKPAFLSGEQKKAIVDLLFKTNRKVTVKQLKEDYFKKIECFDSVEISGV |
| | | EDRFNASLGTYHDLLKIIKDKDFLDNEENEDILEDIVLTLTLFEDREMIEE |
| | | RLKTYAHLFDDKVMKQLKRRRYTGWGRLSRKLINGIRDKQSGKTILDFL |
| | | KSDGFANRNFMQLIHDDSLTFKEDIQKAQVSGQGDSLHEHIANLAGSPAI |
| | | KKGILQTVKVVDELVKVMGRHKPENIVIEMARENQTTQKGQKNSRERM |
| | | KRIEEGIKELGSQILKEHPVENTQLQNEKLYLYYLQNGRDMYVDQELDIN |
| | | RLSDYDVDAIVPQSFLKDDSIDNKVLTRSDKNRGKSDNVPSEEVVKKMKN |
| | | YWRQLLNAKLITQRKFDNLTKAERGGLSELDKAGFIKRQLVETRQITKH |
| | | VAQILDSRMNTKYDENDKLIREVKVITLKSKLVSDFRKDFQFYKVREINN |
| | | YHHAHDAYLNAVVGTALIKKYPKLESEFVYGDYKVYDVRKMIAKSEQEI |
| | | GKATAKYFFYSNIMNFFKTEITLANGEIRKRPLIETNGETGEIVWDKGRD |
| | | FATVRKVLSMPQVNIVKKTEVQTGGFSKESILPKRNSDKLIARKKDWDP |
| | | KKYGGFDSPTVAYSVLVVAKVEKGKSKKLKSVKELLGITIMERSSFEKNP |
| | | IDFLEAKGYKEVKKDLIIKLPKYSLFELENGRKRMLASAGELQKGNELAL |
| | | PSKYVNFLYLASHYEKLKGSPEDNEQKQLFVEQHKHYLDEIIEQISEFSKR |
| | | VILADANLDKVLSAYNKHRDKPIREQAENIIHLFTLTNLGAPAAFKYFDTT |
| | | IDRKRYTSTKEVLDATLIHQSITGLYETRIDLSQLGGDSGGSSGGSKRTAD |
| | | GSEFESPKKKRKVSGGSSGGSTLNIEDEYRLHETSKEPDVSLGSTWLSDFPQAW |
| | | AETGGMGLAVRQAPLIIPLKATSTPVSIKQYPMSQEARLGIKPHIQRLLDQGILVPC |
| | | QSPWNTPLLPVKKPGTNDYRPVQDLREVNKRVEDIHPTVPNPYNLLSGLPPSHQW |
| | | YTVLDLKDAFFCLRLHPTSQPLFAFEWRDPEMGISGQLTWTRLPQGFKNSPTLFN |
| | | EALHRDLADFRIQHPDLILLQYVDDLLLAATSELDCQQGTRALLQTLGNLGYRASA |
| | | KKAQICQKQVKYLGYLLKEGQRWLTEARKETVMGQPTPKTPRQLREFLGKAGFC |
| | | RLFIPGFAEMAAPLYPLTKPGTLFNWGPDQQKAYQEIKQALLTAPALGLPDLTKP |
| | | FELFVDEKQGYAKGVLTQKLGPWRRPVAYLSKKLDPVAAGWPPCLRMVAAIAVLT |
| | | KDAGKLTMGQPLVILAPHAVEALVKQPPDRWLSNARMTHYQALLLDTDRVQFGP |
| | | VVALNPATLLPLPEEGLQHNCLDILAEAHGTRPDLTDQPLPDADHTWYTDGSSLL |
| | | QEGQRKAGAAVTTETEVIWAKALPAGTSAQRAELIALTQALKMAEGKKLNVYTDSR |
| | | YAFATAHIHGEIYRRRGWLTSEGKEIKNKDEILALLKALFLPKRLSIIHCPGHQKGH |
| | | SAEARGNRMADQAARKAAITETPDTSTLLIENSSPSGGSKRTADGSEFESPKKKRKV |
| | | GSGPAAKRVKLD |
| | | KEY: |
| | | N-terminal bipartiteSV40NLS |
| | | CAS9(R221K N394K H840A) |
| | | SGGSx2-met-bpSV40NLS-SGGSx2 LINKER |
| | | M-MLV D200N T306K W313F T330P L603W REVERSE TRANSCRIPTASE |
| | | C-terminal linker-NLS1 |
| | | C-terminal linker-NLS2 |
| 623 | Exemplary prime editor | KRTADGSEFESPKKKRKVDKKYSIGLDIGTNSVGWAVITDEYKVPSKKFKVL |
| | [NLS]-[Cas9((R220K) | GNTDRHSIKKNLIGALLFDSGETAEATRLKRTARRRYTRRKNRICYLQEIFSNE |
| | (R393K) (H839A)]- | MAKVDDSFFHRLEESFLVEEDKKHERHPIFGNIVDEVAYHEKYPTIYHLRKKL |
| | [linker]- | VDSTDKADLRLIYLALAHMIKFRGHFLIEGDLNPDNSDVDKLFIQLVQTYNQL |
| | [MMLV_RT(D200N)(T330P) | FEENPINASGVDAKAILSARLSKSRKLENLIAQLPGEKKNGLFGNLIALSLGLT |
| | (L603W)(T306K)(W313F)]- | PNFKSNFDLAEDAKLQLSKDTYDDDLDNLLAQIGDQYADLFLAAKNLSDAIL |
| | [NLS] without N terminal | LSDILRVNTEITKAPLSASMIKRYDEHHQDLTLLKALVRQQLPEKYKEIFFDQS |
| | methionine | KNGYAGYIDGGASQEEFYKFIKPILEKMDGTEELLVKLKREDLLRKQRTFDN |
| | | GSIPHQIHLGELHAILRRQEDFYPFLKDNREKIEKILTFRIPYYVGPLARGNSRF |
| | | AWMTRKSEETITPWNFEEVVDKGASAQSFIERMTNFDKNLPNEKVLPKHSLL |
| | | YEYFTVYNELTKVKYVTEGMRKPAFLSGEQKKAIVDLLFKTNRKVTVKQLK |
| | | EDYFKKIECFDSVEISGVEDRFNASLGTYHDLLKIIKDKDFLDNEENEDILEDIV |
| | | LTLTLFEDREMIEERLKTYAHLFDDKVMKQLKRRRYTGWGRLSRKLINGIRD |
| | | KQSGKTILDFLKSDGFANRNFMQLIHDDSLTFKEDIQKAQVSGQGDSLHEHIA |
| | | NLAGSPAIKKGILQTVKVVDELVKVMGRHKPENIVIEMARENQTTQKGQKNS |
| | | RERMKRIEEGIKELGSQILKEHPVENTQLQNEKLYLYYLQNGRDMYVDQELD |
| | | INRLSDYDVDAIVPQSFLKDDSIDNKVLTRSDKNRGKSDNVPSEEVVKKMKN |
| | | YWRQLLNAKLITQRKFDNLTKAERGGLSELDKAGFIKRQLVETRQITKHVAQI |
| | | LDSRMNTKYDENDKLIREVKVITLKSKLVSDFRKDFQFYKVREINNYHHAHD |
| | | AYLNAVVGTALIKKYPKLESEFVYGDYKVYDVRKMIAKSEQEIGKATAKYFF |
| | | YSNIMNFFKTEITLANGEIRKRPLIETNGETGEIVWDKGRDFATVRKVLSMPQ |
| | | VNIVKKTEVQTGGFSKESILPKRNSDKLIARKKDWDPKKYGGFDSPTVAYSVL |
| | | VVAKVEKGKSKKLKSVKELLGITIMERSSFEKNPIDFLEAKGYKEVKKDLIIKL |
| | | PKYSLFELENGRKRMLASAGELQKGNELALPSKYVNFLYLASHYEKLKGSPE |
| | | DNEQKQLFVEQHKHYLDEIIEQISEFSKRVILADANLDKVLSAYNKHRDKPIRE |
| | | QAENIIHLFTLTNLGAPAAFKYFDTTIDRKRYTSTKEVLDATLIHQSITGLYETR |
| | | IDLSQLGGDSGGSSGGSKRTADGSEFESPKKKRKVSGGSSGGSTLNIEDEYRL |
| | | HETSKEPDVSLGSTWLSDFPQAWAETGGMGLAVRQAPLIIPLKATSTPVSIKQ |
| | | YPMSQEARLGIKPHIQRLLDQGILVPCQSPWNTPLLPVKKPGTNDYRPVQDLR |
| | | EVNKRVEDIHPTVPNPYNLLSGLPPSHQWYTVLDLKDAFFCLRLHPTSQPLFA |
| | | FEWRDPEMGISGQLTWTRLPQGFKNSPTLFNEALHRDLADFRIQHPDLILLQY |
| | | VDDLLLAATSELDCQQGTRALLQTLGNLGYRASAKKAQICQKQVKYLGYLL |
| | | KEGQRWLTEARKETVMGQPTPKTPRQLREFLGKAGFCRLFIPGFAEMAAPLY |
| | | PLTKPGTLFNWGPDQQKAYQEIKQALLTAPALGLPDLTKPFELFVDEKQGYA |
| | | KGVLTQKLGPWRRPVAYLSKKLDPVAAGWPPCLRMVAAIAVLTKDAGKLT |
| | | MGQPLVILAPHAVEALVKQPPDRWLSNARMTHYQALLLDTDRVQFGPVVAL |
| | | NPATLLPLPEEGLQHNCLDILAEAHGTRPDLTDQPLPDADHTWYTDGSSLLQE |
| | | GQRKAGAAVTTETEVIWAKALPAGTSAQRAELIALTQALKMAEGKKLNVYT |
| | | DSRYAFATAHIHGEIYRRRGWLTSEGKEIKNKDEILALLKALFLPKRLSIIHCPG |
| | | HQKGHSAEARGNRMADQAARKAAITETPDTSTLLIENSSPSGGSKRTADGSEF |
| | | ESPKKKRKVGSGPAAKRVKLD |
| 625 | -N-terminal bpSV40NLS | MKRTADGSEFESPKKKRKV |
| 619 | -CAS9 (R221K N394K | DKKYSIGLDIGTNSVGWAVITDEYKVPSKKFKVLGNTDRHSIKKNLIGALLFD |
| | H840A) | SGETAEATRLKRTARRRYTRRKNRICYLQEIFSNEMAKVDDSFFHRLEESFLV |
| | | EEDKKHERHPIFGNIVDEVAYHEKYPTIYHLRKKLVDSTDKADLRLIYLALAH |
| | | MIKFRGHFLIEGDLNPDNSDVDKLFIQLVQTYNQLFEENPINASGVDAKAILSA |
| | | RLSKSRKLENLIAQLPGEKKNGLFGNLIALSLGLTPNFKSNFDLAEDAKLQLSK |
| | | DTYDDDLDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGASQEEFYK |
| | | FIKPILEKMDGTEELLVKLKREDLLRKQRTFDNGSIPHQIHLGELHAILRRQED |
| | | FYPFLKDNREKIEKILTFRIPYYVGPLARGNSRFAWMTRKSEETITPWNFEEVV |
| | | DKGASAQSFIERMTNFDKNLPNEKVLPKHSLLYEYFTVYNELTKVKYVTEGM |
| | | RKPAFLSGEQKKAIVDLLFKTNRKVTVKQLKEDYFKKIECFDSVEISGVEDRF |
| | | NASLGTYHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDGFANRNF |
| | | MQLIHDDSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGSQILKEH |
| | | PVENTQLQNEKLYLYYLQNGRDMYVDQELDINRLSDYDVDAIVPQSFLKDDS |
| | | IDNKVLTRSDKNRGKSDNVPSEEVVKKMKNYWRQLLNAKLITQRKFDNLTK |
| | | AERGGLSELDKAGFIKRQLVETRQITKHVAQILDSRMNTKYDENDKLIREVKV |
| | | ITLKSKLVSDFRKDFQFYKVREINNYHHAHDAYLNAVVGTALIKKYPKLESEF |
| | | VYGDYKVYDVRKMIAKSEQEIGKATAKYFFYSNIMNFFKTEITLANGEIRKRP |
| | | LIETNGETGEIVWDKGRDFATVRKVLSMPQVNIVKKTEVQTGGFSKESILPKR |
| | | NSDKLIARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRMLASAGE |
| | | LQKGNELALPSKYVNFLYLASHYEKLKGSPEDNEQKQLFVEQHKHYLDEIIEQ |
| | | ISEFSKRVILADANLDKVLSAYNKHRDKPIREQAENIIHLFTLTNLGAPAAFKY |
| | | FDTTIDRKRYTSTKEVLDATLIHQSITGLYETRIDLSQLGGD |
| 664 | -SGGSx2-bpSV40NLS- | SGGSSGGSKRTADGSEFESPKKKRKVSGGSSGGS |
| | SGGSx2 linker | |
| 591 | -MMLV_RT D200N T330P | TLNIEDEYRLHETSKEPDVSLGSTWLSDFPQAWAETGGMGLAVRQAPLIIPLK |
| | L603W T306K W313F | ATSTPVSIKQYPMSQEARLGIKPHIQRLLDQGILVPCQSPWNTPLLPVKKPGTN |
| | | DYRPVQDLREVNKRVEDIHPTVPNPYNLLSGLPPSHQWYTVLDLKDAFFCLR |
| | | LHPTSQPLFAFEWRDPEMGISGQLTWTRLPQGFKNSPTLFNEALHRDLADFRI |
| | | QHPDLILLQYVDDLLLAATSELDCQQGTRALLQTLGNLGYRASAKKAQICQK |
| | | QVKYLGYLLKEGQRWLTEARKETVMGQPTPKTPRQLREFLGKAGFCRLFIPG |
| | | FAEMAAPLYPLTKPGTLFNWGPDQQKAYQEIKQALLTAPALGLPDLTKPFEL |
| | | FVDEKQGYAKGVLTQKLGPWRRPVAYLSKKLDPVAAGWPPCLRMVAAIAV |
| | | LTKDAGKLTMGQPLVILAPHAVEALVKQPPDRWLSNARMTHYQALLLDTDR |
| | | VQFGPVVALNPATLLPLPEEGLQHNCLDILAEAHGTRPDLTDQPLPDADHTW |
| | | YTDGSSLLQEGQRKAGAAVTTETEVIWAKALPAGTSAQRAELIALTQALKMA |
| | | EGKKLNVYTDSRYAFATAHIHGEIYRRRGWLTSEGKEIKNKDEILALLKALFL |
| | | PKRLSIIHCPGHQKGHSAEARGNRMADQAARKAAITETPDTSTLLIENSSP |
| 662 | C-terminal linker-NLS | SGGSKRTADGSEFESPKKKRKV |
| 663 | C-terminal linker-NLS2 | GSGPAAKRVKLD |

PEgRNA for editing of CLRN1 gene

The term “prime editing guide RNA”, or “PEgRNA”, refers to a guide polynucleotide that comprises one or more intended nucleotide edits for incorporation into the target DNA. In some embodiments, the PEgRNA associates with and directs a prime editor to incorporate the one or more intended nucleotide edits into the target gene via prime editing. “Nucleotide edit” or “intended nucleotide edit” refers to a specified deletion of one or more nucleotides at one specific position, insertion of one or more nucleotides at one specific position, substitution of a single nucleotide, or other alterations at one specific position to be incorporated into the sequence of the target gene. Intended nucleotide edit may refer to the edit on the editing template as compared to the sequence on the target strand of the target gene, or may refer to the edit encoded by the editing template on the newly synthesized single stranded DNA that replaces the editing target sequence, as compared to the editing target sequence. In some embodiments, a PEgRNA comprises a spacer sequence that is complementary or substantially complementary to a search target sequence on a target strand of the target gene. In some embodiments, the PEgRNA comprises a gRNA core that associates with a DNA binding domain, e.g., a CRISPR-Cas protein domain, of a prime editor. In some embodiments, the PEgRNA further comprises an extended nucleotide sequence comprising one or more intended nucleotide edits compared to the endogenous sequence of the target gene, wherein the extended nucleotide sequence may be referred to as an extension arm.

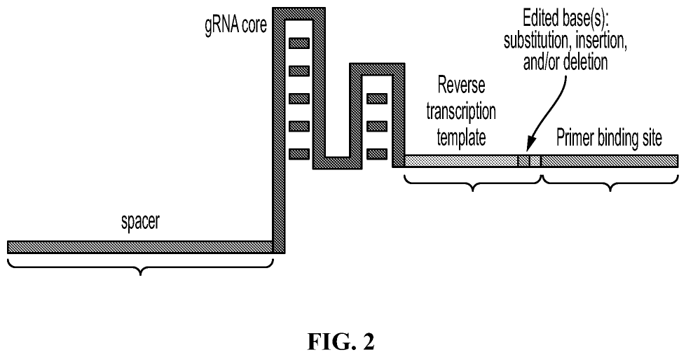

In certain embodiments, the extension arm comprises a primer binding site sequence (PBS) that can initiate target-primed DNA synthesis. In some embodiments, the PBS is complementary or substantially complementary to a free 3′ end on the edit strand of the target gene at a nick site generated by the prime editor. In some embodiments, the extension arm further comprises an editing template that comprises one or more intended nucleotide edits to be incorporated in the target gene by prime editing. In some embodiments, the editing template is a template for an RNA-dependent DNA polymerase domain orpolypeptide of the prime editor, for example, a reverse transcriptase domain. The reverse transcriptase editing template may also be referred to herein as an RT template, or RTT. In some embodiments, the editing template comprises partial complementarity to an editing target sequence in the target gene, e.g., an CLRN1 gene. In some embodiments, the editing template comprises substantial or partial comnplementarⁱty to the editing target sequence except at the position of the intended nucleotide edits to be incorporated into the target gene. An exemplary architecture of a PEgRNA including its components is as demonstrated in FIG. 2.

In some embodiments, a PEgRNA includes only RNA nucleotides and forms an RNA polynucleotide. In some embodiments, a PEgRNA is a chimeric polynucleotide that includes both RNA and DNA nucleotides. For example, a PEgRNA can include DNA in the spacer sequence, the gRNA core, or the extension arm. In some embodiments, a PEgRNA comprises DNA in the spacer sequence. In some embodiments, the entire spacer sequence of a PEgRNA is a DNA sequence. In some embodiments, the PEgRNA comprises DNA in the gRNA core, for example, in a stem region of the gRNA core. In some embodiments, the PEgRNA comprises DNA in the extension arm, for example, in the editing template. An editing template that comprises a DNA sequence may serve as a DNA synthesis template for a DNA polymerase in a prime editor, for example, a DNA-dependent DNA polymerase. Accordingly, the PEgRNA may be a chimeric polynucleotide that comprises RNA in the spacer, gRNA core, and/or the PBS sequences and DNA in the editing template.

Components of a PEgRNA may be arranged in a modular fashion. In some embodiments, the spacer and the extension arm comprising a primer binding site sequence (PBS) and an editing template, e.g., a reverse transcriptase template (RTT), can be interchangeably located in the 5′ portion of the PEgRNA, the 3′ portion of the PEgRNA, or in the middle of the gRNA core. In some embodiments, a PEgRNA comprises a PBS and an editing template sequence in 5′to 3′ order. In some embodiments, the gRNA core of a PEgRNA of this disclosure may be located in between a spacer and an extension arm of the PEgRNA. In some embodiments, the gRNA core of a PEgRNA may be located at the 3′ end of a spacer. In some embodiments, the gRNA core of a PEgRNA may be located at the 5′ end of a spacer. In some embodiments, the gRNA core of a PEgRNA may be located at the 3′ end of an extension arm. In some embodiments, the gRNA core of a PEgRNA may be located at the 5′ end of an extension arm. In some embodiments, the PEgRNA comprises, from 5′ to 3′: a spacer, a gRNA core, and an extension arm. In some embodiments, the PEgRNA comprises, from 5′ to 3′: a spacer, a gRNA core, an editing template, and a PBS. In some embodiments, the PEgRNA comprises, from 5′ to 3′: an extension arm, a spacer, and a gRNA core. In some embodiments, the PEgRNA comprises, from 5′ to 3′: an editing template, a PBS, a spacer, and a gRNA core.

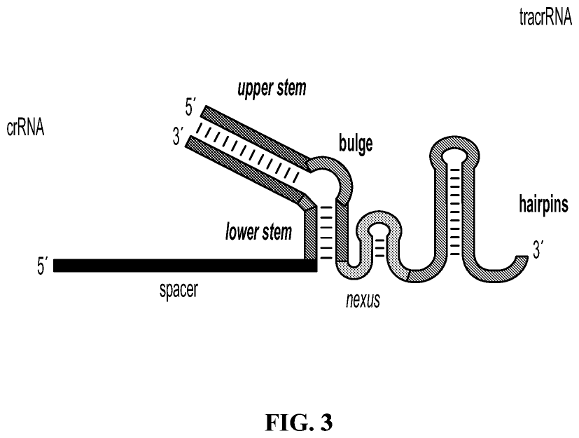

In some embodiments, a PEgRNA comprises a single polynucleotide molecule that comprises the spacer sequence, the gRNA core, and the extension arm. In some embodiments, a PEgRNA comprises multiple polynucleotide molecules, for example, two polynucleotide molecules. In some embodiments, a PEgRNA comprise a first polynucleotide molecule that comprises the spacer and a portion of the gRNA core, and a second polynucleotide molecule that comprises the rest of the gRNA core and the extension arm. In some embodiments, the gRNA core portion in the first polynucleotide molecule and the gRNA core portion in the second polynucleotide molecule are at least partly complementary to each other. In some embodiments, the PEgRNA may comprise a first polynucleotide comprising the spacer and a first portion of a gRNA core comprising, which may also be referred to as a crRNA. In some embodiments, the PEgRNA comprise a second polynucleotide comprising a second portion of the gRNA core and the extension arm, wherein the second portion of the gRNA core may also be referred to as a trans-activating crRNA, or tracr RNA. In some embodiments, the crRNA portion and the tracr RNA portion of the gRNA core are at least partially complementary to each other. In some embodiments, the partially complementary portions of the crRNA and the tracr RNA form a lower stem, a bulge, and an upper stem, as exemplified in FIG. 3.

In some embodiments, a spacer sequence comprises a region that has substantial complementarity to a search target sequence on the target strand of a double stranded target DNA, e.g., an CLRN1 gene. In some embodiments, the spacer sequence of a PEgRNA is identical or substantially identical to a protospacer sequence on the edit strand of the target gene (except that the protospacer sequence comprises thymine and the spacer sequence may comprise uracil). In some embodiments, the spacer sequence is at least about 70%, 75%, 80%, 85%, 90%, 95%, or 100% complementary to a search target sequence in the target gene. In some embodiments, the spacer comprises is substantially complementary to the search target sequence.

In some embodiments, the length of the spacer varies from at least about 10 to about 100 nucleotides. In some embodiments, the spacer is 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides in length. In some embodiments, the spacer is from 15 nucleotides to 30 nucleotides in length, 15 to 25 nucleotides in length, 18 to 22 nucleotides in length, 10 to 20 nucleotides in length, or 20 to 30 nucleotides in length. In some embodiments, the spacer is 17 to 22 nucleotides in length, e.g., about 17, 18, 19, 20, 21, or 22 nucleotides in length. In some embodiments, the spacer is 20 nucleotides in length.

As used herein in a PEgRNA or a nick guide RNA sequence, or fragments thereof such as a spacer, PBS, or RTT sequence, unless indicated otherwise, it should be appreciated that the letter “T” or “thymine” indicates a nucleobase in a DNA sequence that encodes the PEgRNA or guide RNA sequence, and is intended to refer to an uracil (U) nucleobase of the PEgRNA or guide RNA or any chemically modified uracil nucleobase known in the art, such as 5-methoxyuracil.

The extension arm of a PEgRNA may comprise a primer binding site (PBS) and an editing template (e.g., an RTT). The extension arm may be partially complementary to the spacer. In some embodiments, the editing template (e.g., RTT) is partially complementary to the spacer. In some embodiments, the editing template (e.g., RTT) and the primer binding site (PBS) are each partially complementary to the spacer.

An extension arm of a PEgRNA may comprise a primer binding site sequence (PBS, or PBS sequence) that comprises complementarity to and can hybridize with a free 3′ end of a single stranded DNA in the target gene (e.g., the CLRN1 gene) generated by nicking with a prime editor at the nick site on the PAM strand.

The length of the primer binding site (PBS) sequence may vary depending on, e.g., the prime editor components, the search target sequence and other components of the PEgRNA. In some embodiments, the PBS is about 3 to 19 nucleotides in length. In some embodiments, the PBS is about 3 to 17 nucleotides in length. In some embodiments, the PBS is about 4 to 16 nucleotides, about 6 to 16 nucleotides, about 6 to 18 nucleotides, about 6 to 20 nucleotides, about 8 to 20 nucleotides, about 10 to 20 nucleotides, about 12 to 20 nucleotides, about 14 to 20 nucleotides, about 16 to 20 nucleotides, or about 18 to 20 nucleotides in length. In some embodiments, the PBS is 8 to 17 nucleotides in length. In some embodiments, the PBS is 8 to 16 nucleotides in length. In some embodiments, the PBS is 8 to 15 nucleotides in length. In some embodiments, the PBS is 8 to 14 nucleotides in length. In some embodiments, the PBS is 8 to 13 nucleotides in length. In some embodiments, the PBS is 8 to 12 nucleotides in length. In some embodiments, the PBS is 8 to 11 nucleotides in length. In some embodiments, the PBS is 8 to 10 nucleotides in length. In some embodiments, the PBS is 8 or 9 nucleotides in length. In some embodiments, the PBS is 16 or 17 nucleotides in length. In some embodiments, the PBS is 15 to 17 nucleotides in length. In some embodiments, the PBS is 14 to 17 nucleotides in length. In some embodiments, the PBS is 13 to 17 nucleotides in length. In some embodiments, the PBS is 12 to 17 nucleotides in length. In some embodiments, the PBS is 11 to 17 nucleotides in length. In some embodiments, the PBS is 10 to 17 nucleotides in length. In some embodiments, the PBS is 9 to 17 nucleotides in length. In some embodiments, the PBS is about 7 to 15 nucleotides in length. In some embodiments, the PBS is 6, 7, 8, 9, 10, 11, 12, 13, 14, 15, 16, 17, 18, or 19 nucleotides in length. In some embodiments, the PBS is 8 to 14 nucleotides in length. For example, the PBS can be 8, 9, 10, 11, 12, 13, or 14 nucleotides in length. In some embodiments, the PBS is 11 or 12 nucleotides in length. In some embodiments, the PBS is 11 to 13 nucleotides in length. In some embodiments, the PBS is 11 to 14 nucleotides in length. In some cases, a PBS length of no more than 3 nucleotides less than the PEgRNA spacer is chosen. For example, for PEgRNA spacers that are 16 to 22 nucleotides in length, a PBS length of up to 19 nucleotides, e.g., 3 to 19, 5 to 19, or 7 to 19 nucleotides, may be chosen. In some embodiments, the PBS is 5 to 19 nucleotides in length.

The PBS may be complementary or substantially complementary to a DNA sequence in the edit strand of the target gene. By annealing with the edit strand at a free hydroxy group, e.g., a free 3′ end generated by prime editor nicking, the PBS may initiate synthesis of a new single stranded DNA encoded by the editing template at the nick site. In some embodiments, the PBS is at least about 70%, 75%, 80%, 85%, 90%, 95%, or 100% complementary to a region of the edit strand of the target gene (e.g., the CLRN1 gene). In some embodiments, the PBS is perfectly complementary, or 100% complementary, to a region of the edit strand of the target gene (e.g., the CLRN1 gene).

An extension arm of a PEgRNA may comprise an editing template that serves as a DNA synthesis template for the DNA polymerase in a prime editor during prime editing.

The length of an editing template may vary depending on, e.g., the prime editor components, the search target sequence and other components of the PEgRNA. In some embodiments, the editing template serves as a DNA synthesis template for a reverse transcriptase, and the editing template is referred to as a reverse transcription editing template (RTT).

The editing template (e.g., RTT), in some embodiments, is 2, 3, 4, 5, 6, 7, 8, 9, 10, 11, 12, 13, 14, 15, 16, 17, 18, 19, 20, 21, 22, 23, 24, or 25 nucleotides in length. In some embodiments, the RTT is 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides in length. In some embodiments, the RTT is 10 to 110 nucleotides in length. In some embodiments, the RTT is 10 to 109, 10 to 108, 10 to 107, 10 to 106, 10 to 105, 10 to 104, 10 to 103, 10 to 102, or 10 to 101 nucleotides in length. In some embodiments, the RTT is from 14 to 34 nucleotides in length. In some embodiments, the RTT is from 18 to 22 nucleotides in length. In some embodiments, the RTT is at least 8 and no more than 50 nucleotides in length. In some embodiments, the RTT is at least 8 and no more than 25 nucleotides in length. In some embodiments, the RTT is about 10 to about 20 nucleotides in length. In some embodiments, the RTT is about 11, 12, 13, 14, 15, 16, 17, 18, or 19 nucleotides in length. In some embodiments, the RTT is 11 to 17 nucleotides in length. In some embodiments, the RTT is 12 to 17 nucleotides in length. In some embodiments, the RTT is 12 to 16 nucleotides in length. In some embodiments, the RTT is 13 to 17 nucleotides in length. In some embodiments, the RTT is 11, 12, 13, 14, 15, 16, or 17 nucleotides in length. In some embodiments the RTT is 12 nucleotides in length. In some embodiments the RTT is 16 nucleotides in length. In some embodiments the RTT is 17 nucleotides in length.

In some embodiments, the RTT has a length of 44 nucleotides or less. In some embodiments, the RTT has a length of 34 nucleotides or less. In some embodiments, the RTT has a length of 22 nucleotides or less.

In some embodiments, the editing template (e.g., RTT) sequence is about 70%, 75%, 80%, 85%, 90%, 95%, or 99% complementary to the editing target sequence on the edit strand of the target gene (e.g., the CLRN1 gene). In some embodiments, the editing template sequence (e.g., RTT) is substantially complementary to the editing target sequence. In some embodiments, the editing template sequence (e.g., RTT) is complementary to the editing target sequence except at positions of the intended nucleotide edits to be incorporated int the target gene. In some embodiments, the editing template comprises a nucleotide sequence comprising about 85% to about 95% complementarity to an editing target sequence in the edit strand in the target gene (e.g., the CLRN1 gene). In some embodiments, the editing template comprises about 86%, about 87%, about 88%, about 89%, about 90%, about 91%, about 92%, about 93%, about 94%, about 95%, about 96%, about 97%, about 98%, or about 99% complementarity to an editing target sequence in the edit strand of the target gene (e.g., the CLRN1 gene).

An intended nucleotide edit in an editing template of a PEgRNA may comprise various types of alterations as compared to the target gene sequence. In some embodiments, the nucleotide edit is a single nucleotide substitution as compared to the target gene sequence. In some embodiments, the nucleotide edit is a deletion as compared to the target gene sequence. In some embodiments, the nucleotide edit is an insertion as compared to the target gene sequence. In some embodiments, the editing template comprises one to ten intended nucleotide edits as compared to the target gene sequence. In some embodiments, the editing template comprises one or more intended nucleotide edits as compared to the target gene sequence. In some embodiments, the editing template comprises two or more intended nucleotide edits as compared to the target gene sequence. In some embodiments, the editing template comprises three or more intended nucleotide edits as compared to the target gene sequence. In some embodiments, the editing template comprises four or more, five or more, or six or more intended nucleotide edits as compared to the target gene sequence. In some embodiments, the editing template comprises two single nucleotide substitutions, insertions, deletions, or any combination thereof, as compared to the target gene sequence. In some embodiments, the editing template comprises three single nucleotide substitutions, insertions, deletions, or any combination thereof, as compared to the target gene sequence. In some embodiments, the editing template comprises four, five, or six single nucleotide substitutions, insertions, deletions, or any combination thereof, as compared to the target gene sequence. In some embodiments, a nucleotide substitution comprises an adenine (A)-to-thymine (T) substitution. In some embodiments, a nucleotide substitution comprises an A-to-guanine (G) substitution. In some embodiments, a nucleotide substitution comprises an A-to-cytosine (C) substitution. In some embodiments, a nucleotide substitution comprises a T-A substitution. In some embodiments, a nucleotide substitution comprises a T-G substitution. In some embodiments, a nucleotide substitution comprises a T-C substitution. In some embodiments, a nucleotide substitution comprises a G-to-A substitution. In some embodiments, a nucleotide substitution comprises a G-to-T substitution. In some embodiments, a nucleotide substitution comprises a G-to-C substitution. In some embodiments, a nucleotide substitution comprises a C-to-A substitution. In some embodiments, a nucleotide substitution comprises a C-to-T substitution. In some embodiments, a nucleotide substitution comprises a C-to-G substitution.

In some embodiments, a nucleotide insertion is at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, or at least 20 nucleotides in length. In some embodiments, a nucleotide insertion is from 1 to 2 nucleotides, from 1 to 3 nucleotides, from 1 to 4 nucleotides, from 1 to 5 nucleotides, form 2 to 5 nucleotides, from 3 to 5 nucleotides, from 3 to 6 nucleotides, from 3 to 8 nucleotides, from 4 to 9 nucleotides, from 5 to 10 nucleotides, from 6 to 11 nucleotides, from 7 to 12 nucleotides, from 8 to 13 nucleotides, from 9 to 14 nucleotides, from 10 to 15 nucleotides, from 11 to 16 nucleotides, from 12 to 17 nucleotides, from 13 to 18 nucleotides, from 14 to 19 nucleotides, from 15 to 20 nucleotides in length. In some embodiments, a nucleotide insertion is a single nucleotide insertion. In some embodiments, a nucleotide insertion comprises insertion of two nucleotides.

The editing template of a PEgRNA may comprise one or more intended nucleotide edits, compared to the target gene (e.g., a CLRN1 gene) to be edited. Position of the intended nucleotide edit(s) relevant to other components of the PEgRNA, or to particular nucleotides (e.g., mutations) in the target gene (e.g., CLRN1 gene) may vary. In some embodiments, the nucleotide edit is in a region of the PEgRNA corresponding to or homologous to the protospacer sequence. In some embodiments, the nucleotide edit is in a region of the PEgRNA corresponding to a region of the target gene (e.g., the CLRN1 gene) outside of the protospacer sequence.

By “upstream” and “downstream” it is intended to define relevant positions at least two regions or sequences in a nucleic acid molecule orientated in a 5′-to-3′ direction. For example, a first sequence is upstream of a second sequence in a DNA molecule where the first sequence is positioned 5′ to the second sequence. Accordingly, the second sequence is downstream of the first sequence.

In some embodiments, the position of a nucleotide edit incorporation in the target gene may be referred to based on the position of the nick site. In some embodiments, position of an intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 110, 120, 130, 140, or 150 nucleotides apart from the nick site. In some embodiments, position of an intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95,100,110, 120, 130, 140, or 150 nucleotides downstream of the nick site on the PAM strand (or the non-target strand, or the edit strand) of the double stranded target DNA. In some embodiments, position of the intended nucleotide edit in the editing template may be referred to by aligning the editing template with the partially complementary editing target sequence on the edit strand, and referring to nucleotide positions on the editing strand where the intended nucleotide edit is incorporated. Accordingly, in some embodiments, a nucleotide edit in an editing template is at a position corresponding to a position about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95,100,110, 120, 130, 140, or 150 nucleotides apart from the nick site.

In some embodiments, a nucleotide edit in an editing template is at a position corresponding to a position about 0 to 2 nucleotides, 0 to 4 nucleotides, 0 to 6 nucleotides, 0 to 8 nucleotides, 0 to 10 nucleotides, 2 to 4 nucleotides, 2 to 6 nucleotides, 2 to 8 nucleotides, 2 to 10 nucleotides, 2 to 12 nucleotides, 4 to 6 nucleotides, 4 to 8 nucleotides, 4 to 10 nucleotides, 4 to 12 nucleotides, 4 to 14 nucleotides, 6 to 8 nucleotides, 6 to 10 nucleotides, 6 to 12 nucleotides, 6 to 14 nucleotides, 6 to 16 nucleotides, 8 to 10 nucleotides, 8 to 12 nucleotides, 8 to 14 nucleotides, 8 to 16 nucleotides, 8 to 18 nucleotides, 10 to 12 nucleotides, 10 to 14 nucleotides, 10 to 16 nucleotides, 10 to 18 nucleotides, 10 to 20 nucleotides, 12 to 14 nucleotides, 12 to 16 nucleotides, 12 to 18 nucleotides, 12 to 20 nucleotides, 12 to 22 nucleotides, 14 to 16 nucleotides, 14 to 18 nucleotides, 14 to 20 nucleotides, 14 to 22 nucleotides, 14 to 24 nucleotides, 16 to 18 nucleotides, 16 to 20 nucleotides, 16 to 22 nucleotides, 16 to 24 nucleotides, 16 to 26 nucleotides, 18 to 20 nucleotides, 18 to 22 nucleotides, 18 to 24 nucleotides, 18 to 26 nucleotides, 18 to 28 nucleotides, 20 to 22 nucleotides, 20 to 24 nucleotides, 20 to 26 nucleotides, 20 to 28 nucleotides, 20 to 30 nucleotides, 30 to 40 nucleotides, 40 to 50 nucleotides, 50 to 60 nucleotides, 60 to 70 nucleotides, 70 to 80 nucleotides, 80 to 90 nucleotides, 90 to 100 nucleotides, 100 to 110 nucleotides, 110 to 120 nucleotides, 120 to 130 nucleotides, 130 to 140 nucleotides, or 140 to 150 nucleotides apart from the nick site. In some embodiments, when referred to in the context of the PAM strand (or the non-target strand, or the edit strand), a nucleotide edit in an editing template is at a position corresponding to a position about 0 to 2 nucleotides, 0 to 4 nucleotides, 0 to 6 nucleotides, 0 to 8 nucleotides, 0 to 10 nucleotides, 2 to 4 nucleotides, 2 to 6 nucleotides, 2 to 8 nucleotides, 2 to 10 nucleotides, 2 to 12 nucleotides, 4 to 6 nucleotides, 4 to 8 nucleotides, 4 to 10 nucleotides, 4 to 12 nucleotides, 4 to 14 nucleotides, 6 to 8 nucleotides, 6 to 10 nucleotides, 6 to 12 nucleotides, 6 to 14 nucleotides, 6 to 16 nucleotides, 8 to 10 nucleotides, 8 to 12 nucleotides, 8 to 14 nucleotides, 8 to 16 nucleotides, 8 to 18 nucleotides, 10 to 12 nucleotides, 10 to 14 nucleotides, 10 to 16 nucleotides, 10 to 18 nucleotides, 10 to 20 nucleotides, 12 to 14 nucleotides, 12 to 16 nucleotides, 12 to 18 nucleotides, 12 to 20 nucleotides, 12 to 22 nucleotides, 14 to 16 nucleotides, 14 to 18 nucleotides, 14 to 20 nucleotides, 14 to 22 nucleotides, 14 to 24 nucleotides, 16 to 18 nucleotides, 16 to 20 nucleotides, 16 to 22 nucleotides, 16 to 24 nucleotides, 16 to 26 nucleotides, 18 to 20 nucleotides, 18 to 22 nucleotides, 18 to 24 nucleotides, 18 to 26 nucleotides, 18 to 28 nucleotides, 20 to 22 nucleotides, 20 to 24 nucleotides, 20 to 26 nucleotides, 20 to 28 nucleotides, 20 to 30 nucleotides, 30 to 40 nucleotides, 40 to 50 nucleotides, 50 to 60 nucleotides, 60 to 70 nucleotides, 70 to 80 nucleotides, 80 to 90 nucleotides, 90 to 100 nucleotides, 100 to 110 nucleotides, 110 to 120 nucleotides, 120 to 130 nucleotides, 130 to 140 nucleotides, or 140 to 150 nucleotides downstream from the nick site.

The relative positions of the intended nucleotide edit(s) and nick site may be referred to by numbers. For example, in some embodiments, the nucleotide immediately downstream of the nick site on a PAM strand (or the non-target strand, or the edit strand) may be referred to as at position 0. The nucleotide immediately upstream of the nick site on the PAM strand (or the non-target strand, or the edit strand) may be referred to as at position −1. The nucleotides downstream of position 0 on the PAM strand may be referred to as at positions+1, +2, +3, +4, . . . +n, and the nucleotides upstream of position −1 on the PAM strand may be referred to as at positions −2, −3, −4, . . . , −n. Accordingly, in some embodiments, the nucleotide in the editing template that corresponds to position 0 when the editing template is aligned with the partially complementary editing target sequence by complementarity may also be referred to as position 0 in the editing template, the nucleotides in the editing template corresponding to the nucleotides at positions+1, +2, +3, +4, . . . , +n on the PAM strand of the double stranded target DNA may also be referred to as at positions+1, +2, +3, +4, . . . , +n in the editing template, and the nucleotides in the editing template corresponding to the nucleotides at positions −1, −2, −3, −4, . . . , −n on the PAM strand on the double stranded target DNA may also be referred to as at positions −1, −2, −3, −4, . . . , −n on the editing template, even though when the PEgRNA is viewed as a standalone nucleic acid, positions+1, +2, +3, +4, . . . , +n are 5′ of position 0 and positions −1, −2, −3, −4, . . . -n are 3′ of position 0 in the editing template. In some embodiments, an intended nucleotide edit is at position+n of the editing template relative to position 0. Accordingly, the intended nucleotide edit may be incorporated at position+n of the PAM strand of the double stranded target DNA (and subsequently, the target strand of the double stranded target DNA) by prime editing, wherein n is an integer no less than 0. The corresponding positions of the intended nucleotide edit incorporated in the target gene may also be referred to based on the nicking position generated by a prime editor based on sequence homology and complementarity. For example, in embodiments, the distance between the nucleotide edit to be incorporated into the target gene (e.g., CLRN1 gene) and the nick site (also referred to as “the nick to edit distance”) may be determined by the position of the nick site and the position of the nucleotide(s) corresponding to the intended nucleotide edit(s), for example, by identifying sequence complementarity between the spacer and the search target sequence and sequence complementarity between the editing template and the editing target sequence. In certain embodiments, the position of the nucleotide edit can be in any position downstream of the nick site on the edit strand (or the PAM strand). As used herein, the distance between the nick site and the nucleotide edit, for example, where the nucleotide edit comprises an insertion or deletion, refers to the 5′ most position of the nucleotide edit for a nick that creates a 3′ free end on the edit strand (i.e., the “near position” of the nucleotide edit to the nick site). In some embodiments, the nick-to-edit distance is 2 to 106 nucleotides. In some embodiments, the nick-to-edit distance is 2 to 105, 2 to 104, 2 to 103, 2 to 102, 2 to 101, 2 to 100, 2 to 99, 2 to 98, or 2 to 97 nucleotides. In some embodiments, the nick-to-edit distance is 2 to 90, 2 to 80, 2 to 70, 2 to 60, 2 to 50, 2 to 40, or 2 to 30 nucleotides. In some embodiments, the nick-to-edit distance is 2 to 25, 2 to 20, 2 to 15, or 2 to 10 nucleotides. In some embodiments, the nick-to-edit distance is 2, 3, 4, 5, 6, or 7 nucleotides in length.

The RTT length and the nick-to-edit distance relate to the length of the portion of the RTT that is upstream of (i.e. 5′ to) the 5′-most edit in the RTT and is complementary to the edit strand. In some embodiments, the editing template comprises at least 4 contiguous nucleotides of complementarity with the edit strand wherein the at least 4 nucleotides contiguous are located upstream of the 5′ most edit in the editing template. In some embodiments, the editing template comprises at least 5, 6, 7, 8, 9, 10, 11, 12, 13, 14, 15, 16, 17, 18, 19, or more contiguous nucleotides of complementarity with the edit strand wherein the at least 5, 6, 7, 8, 9, 10, 11, 12, 13, 14, 15, 16, 17, 18, 19, or more contiguous nucleotides are located upstream of the 5′ most edit in the editing template. In some embodiments, the editing template comprises 20-25, 25-30, 30-35, 35-40, 45-45, or 45-50 contiguous nucleotides of complementarity with the edit strand wherein the 20-25, 25-30, 30-35, 35-40, 45-45, or 45-50 or more contiguous nucleotides are located upstream of the 5′ most edit in the editing template. In some embodiments, the editing template comprises 9-14 contiguous nucleotides of complementarity with the edit strand wherein the 9-14 contiguous nucleotides are located upstream of the 5′ most edit in the editing template. In some embodiments, the editing template comprises 6-10 contiguous nucleotides of complementarity with the edit strand wherein the 6-10 contiguous nucleotides are located upstream of the 5′ most edit in the editing template. In some embodiments, the editing template comprises 10 contiguous nucleotides of complementarity with the edit strand wherein the 10 contiguous nucleotides are located upstream of the 5′ most edit in the editing template. In some embodiments, the editing template comprises 9 contiguous nucleotides of complementarity with the edit strand wherein the 9 contiguous nucleotides are located upstream of the 5′ most edit in the editing template.

When referred to within the PEgRNA, positions of the one or more intended nucleotide edits may be referred to relevant to components of the PEgRNA. For example, an intended nucleotide edit may be 5′ or 3′ to the PBS. In some embodiments, a PEgRNA comprises the structure, from 5′ to 3′: a spacer, a gRNA core, an editing template, and a PBS. In some embodiments, the intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides upstream to the 5′ most nucleotide of the PBS. In some embodiments, the intended nucleotide edit is 0 to 2 nucleotides, 0 to 4 nucleotides, 0 to 6 nucleotides, 0 to 8 nucleotides, 0 to 10 nucleotides, 2 to 4 nucleotides, 2 to 6 nucleotides, 2 to 8 nucleotides, 2 to 10 nucleotides, 2 to 12 nucleotides, 4 to 6 nucleotides, 4 to 8 nucleotides, 4 to 10 nucleotides, 4 to 12 nucleotides, 4 to 14 nucleotides, 6 to 8 nucleotides, 6 to 10 nucleotides, 6 to 12 nucleotides, 6 to 14 nucleotides, 6 tol6 nucleotides, 8 to 10 nucleotides, 8 to 12 nucleotides, 8 to 14 nucleotides, 8 to 16 nucleotides, 8 to 18 nucleotides, 10 to 12 nucleotides, 10 to 14 nucleotides, 10 to 16 nucleotides, 10 to 18 nucleotides, 10 to 20 nucleotides, 12 to 14 nucleotides, 12 to 16 nucleotides, 12 to 18 nucleotides, 12 to 20 nucleotides, 12 to 22 nucleotides, 14 to 16 nucleotides, 14 to 18 nucleotides, 14 to 20 nucleotides, 14 to 22 nucleotides, 14 to 24 nucleotides, 16 to 18 nucleotides, 16 to 20 nucleotides, 16 to 22 nucleotides, 16 to 24 nucleotides, 16 to 26 nucleotides, 18 to 20 nucleotides, 18 to 22 nucleotides, 18 to 24 nucleotides, 18 to 26 nucleotides, 18 to 28 nucleotides, 20 to 22 nucleotides, 20 to 24 nucleotides, 20 to 26 nucleotides, 20 to 28 nucleotides, or 20 to 30 nucleotides upstream to the 5′ most nucleotide of the PBS.

The corresponding positions of the intended nucleotide edit incorporated in the target gene may also be referred to based on the nicking position generated by a prime editor based on sequence homology and complementarity. For example, in embodiments, the distance between the nucleotide edit to be incorporated into the target gene (e.g., CLRN1 gene) and the nick site (also referred to as the “nick to edit distance”) may be determined by the position of the nick site and the position of the nucleotide(s) corresponding to the intended nucleotide edit(s), for example, by identifying sequence complementarity between the spacer and the search target sequence and sequence complementarity between the editing template and the editing target sequence. In certain embodiments, the position of the nucleotide edit can be in any position downstream of the nick site on the edit strand (or the PAM strand) generated by the prime editor, such that the distance between the nick site and the intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. In some embodiments, the position of the nucleotide edit is 0, 1,2,3,4,5,6,7, 8,9, 10, 11, 12, 13, 14, 15, 16, 17, 18, 19, 20, 21, 22, 23, 24, 25, 26, 27, 28, 29, or 30 nucleotides upstream of the nick site on the edit strand. In some embodiments, the position of the nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides downstream of the nick site on the edit strand. In some embodiments, the position of the nucleotide edit is 0 base pair from the nick site on the edit strand, that is, the editing position is at the same position as the nick site. As used herein, the distance between the nick site and the nucleotide edit, for example, where the nucleotide edit comprises an insertion or deletion, refers to the 5′ most position of the nucleotide edit for a nick that creates a 3′ free end on the edit strand (i.e., the “near position” of the nucleotide edit to the nick site). Similarly, as used herein, the distance between the nick site and a PAM position edit, for example, where the nucleotide edit comprises an insertion, deletion, or substitution of two or more contiguous nucleotides, refers to the 5′ most position of the nucleotide edit and the 5′ most position of the PAM sequence.

In some embodiments, the editing template extends beyond a nucleotide edit to be incorporated to the target CLRN1 gene sequence. For example, in some embodiments, the editing template comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides 3′ to the nucleotide edit to be incorporated to the target gene sequence (e.g., CLRN1 gene sequence). In some embodiments, the editing template comprises at least 4 to 30 base pairs 3′ to the nucleotide edit to be incorporated to the target CLRN1 gene sequence. In some embodiments, the editing template comprises at least 4 to 25 base pairs 3′ to the nucleotide edit to be incorporated to the target CLRN1 gene sequence. In some embodiments, the editing template comprises at least 4 to 20 base pairs 3′ to the nucleotide edit to be incorporated to the target CLRN1 gene sequence. In some embodiments, the editing template comprises at least 4 to 30 base pairs 5′ to the nucleotide edit to be incorporated to the target CLRN1 gene sequence. In some embodiments, the editing template comprises at least 4 to 25 base pairs 5′ to the nucleotide edit to be incorporated to the target CLRN1 gene sequence. In some embodiments, the editing template comprises at least 4 to 20 base pairs 5′ to the nucleotide edit to be incorporated to the target CLRN1 gene sequence.

In some embodiments, the editing template can comprise a second edit relative to a target sequence. The second edit can be designed to mutate or otherwise silence a PAM sequence such that a corresponding nucleic acid guided nuclease or CRISPR nuclease is no longer able to cleave the target sequence (such edits referred to as “PAM silencing edits).

Without wishing to be bound by any particular theory, PAM silencing edits may prevent the Cas, e.g., Cas9, nickase, from re-nicking the edit strand before the edit is incorporated in the target strand, therefore improving prime editing efficiency. In some embodiments, a PAM silencing edit is a synonymous edit that does not alter the amino acid sequence encoded by the target gene after incorporation of the edit. In some embodiments, a PAM silencing edit is at a position corresponding to a non-coding region, e.g., an intron, of a target gene (e.g., CLRN1 gene). In some embodiments, the edits in an intron of a target gene is not at a position that corresponds to intron-exon junction and the edit does not affect transcript splicing.

In some embodiments, the length of the editing template is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides longer than the nick to edit distance. In some embodiments, for example, the nick to edit distance is 8 nucleotides, and the editing template is 10 to 15, 10 to 20, 10 to 25, 10 to 30, 10 to 35, 10 to 40, 10 to 45, 10 to 50, 10 to 55, 10 to 60, 10 to 65, 10 to 70, 10 to 75, or 10 to 80 nucleotides in length. In some embodiments, the nick to edit distance is 22 nucleotides, and the editing template is 24 to 28, 24 to 30, 24 to 32, 24 to 34, 24 to 36, 24 to 37, 24 to 38, 24 to 40, 24 to 45, 24 to 50, 24 to 55, 24 to 60, 24 to 65, 24 to 70, 24 to 75, 24 to 80, 24 to 85, 24 to 90, 24 to 95, 24 to 100, 24 to 105, 24 to 100, 24 to 105, or 24 to 110 nucleotides in length.

In some embodiments, the editing template comprises an adenine at the first nucleobase position (e.g., for a PEgRNA following 5′-spacer-gRNA core-RTT-PBS-3′ orientation, the 5′ most nucleobase is the “first base”). In some embodiments, the editing template comprises a guanine at the first nuclcobase position (e.g., for a PEgRNA following 5′-spacer-gRNA core-RTT-PBS-3′ orientation, the 5′ most nucleobase is the “first base”). In some embodiments, the editing template comprises an uracil at the first nucleobase position (e.g., for a PEgRNA following 5′-spacer-gRNA core-RTT-PBS-3′ orientation, the₅′ most nucleobase is the “first base”). In some embodiments, the editing template comprises a cytosine at the first nucleobase position (e.g., for a PEgRNA following 5′-spacer-gRNA core-RTT-PBS-3′ orientation, the 5′ most nucleobase is the “first base”). In some embodiments, the editing template does not comprise a cytosine at the first mucleobase position (e.g., for a PEgRNA following 5′-spacer-gRNA core-RTT-PBS-3′ orientation, the 5′ most nucleobase is the “first base”).

The editing template of a PEgRNA may encode a new single stranded DNA (e.g., by reverse transcription) to replace an editing target sequence in the target gene. In some embodiments, the editing target sequence in the edit strand of the target gene is replaced by the newly synthesized strand, and the nucleotide edit(s) are incorporated in the region of the target gene. In some embodiments, the target gene is an CLRN1 gene. In some embodiments, the editing template of the PEgRNA encodes a newly synthesized single stranded DNA that comprises a wild type gene sequence e.g., CLRN1 gene sequence. In some embodiments, the newly synthesized DNA strand replaces the editing target sequence in the target gene (e.g., CLRN1 gene), wherein the editing target sequence (or the endogenous sequence complementary to the editing target sequence on the target strand of the target gene (e.g., CLRN1 gene) comprises a mutation or a nucleotide alteration compared to a reference gene, e.g., a wild type CLRN1 gene. In some embodiments, the mutation is associated with retinal degenerative disease, such as Usher Syndrome type 3. In some embodiments, the newly synthesized single stranded DNA encoded by the editing target sequence replaces the editing target sequence, and corrects the mutation in the editing target sequence of the target gene (e.g., CLRN1 gene).

In some embodiments, the editing target sequence comprises a mutation in exon 0, exon 1, exon 2, exon 3, or exon 4 of the CLRN1 gene, as compared to a wild type CLRN1 gene. In some embodiments, the editing target sequence comprises a mutation at an exon/intron junction of the CLRN1 gene as compared to exon/intron junction of a wild type CLRN1 gene.

Unless otherwise indicated, references to nucleotide positions in human chromosomes are as set forth in human genome assembly consortium Human build 38 (GRCh38), GenBank accession GCF_000001405.38.

In some embodiments, the editing target sequence comprises a mutation in exon 0 of the CLRN1 gene as compared to a wild type CLRN1 gene. In some embodiments, the editing target sequence comprises a mutation that is located at position 144 of the coding sequence of the clarin-1 protein. In some embodiments, the editing target sequence comprises a c.144T->G mutation (on the sense strand) or a A->C mutation (on the antisense strand) at position 144 of the coding sequence of the clarin-1 protein.

In some embodiments, the editing template comprises one or more intended nucleotide edits compared to the sequence on the target strand of the target gene (e.g., CLRN1 gene) that is complementary to the editing target sequence. The one or more intended nucleotide edits can be a single nucleotide substitution, polynucleotide substitution, nucleotide insertion, or nucleotide deletion. In some embodiments, the intended nucleotide edit in the editing template comprises a single nucleotide substitution, polynucleotide substitution, nucleotide insertion, or nucleotide deletion compared to the sequence on the target strand of the target gene (e.g., CLRN1 gene) that is complementary to the editing target at a position corresponding to a mutation in the target gene, wherein the editing target sequence is on the sense strand of the target gene. In some embodiments, the intended nucleotide edit in the editing template comprises a single nucleotide substitution, polynucleotide substitution, nucleotide insertion, or nucleotide deletion compared to the sequence on the target strand of the target gene that is complementary to the editing target at a position corresponding to a mutation in the target gene, wherein the editing target sequence is on the antisense strand of the target gene (e.g., CLRN1 gene). In some embodiments, the editing template encodes a single stranded DNA that comprises one or more intended nucleotide edits compared to the editing target sequence. In some embodiments, the single stranded DNA replaces the editing target sequence by prime editing, thereby incorporating the one or more intended nucleotide edits. In some embodiments, the one or more intended nucleotide edits comprises a G-T substitution at a position corresponding to position 144 of the coding sequence of the clarin-1 protein compared to the editing target sequence. In some embodiments, the one or more intended nucleotide edits comprises a C-A substitution in the anti-sense strand at a position corresponding to position 144 of the coding sequence of the clarin-1 protein compared to the editing target sequence. In some embodiments, incorporation of the one or more intended nucleotide edits corrects the mutation in the editing target sequence to wild type nucleotides at corresponding positions in the CLRN1 gene. As used herein, “correcting” a mutation means restoring a wild type sequence at the place of the mutation in the double stranded target DNA, e.g. target gene, by prime editing. In some embodiments, the editing template comprises and/or encodes a wild type target gene sequence (e.g., wild type CLRN1 gene sequence).

In some embodiments, incorporation of the one or more intended nucleotide edits does not correct the mutation in the editing target sequence to wild type sequence but allows for expression of a functional clarin-1 protein encoded by the CLRN1 gene. For example, in some embodiments, incorporation of the one or more intended nucleotide edits results in one or more codons that are different from a wild type codon but encode one or more amino acids same as the wild type clarin-1 protein. In some embodiments, incorporation of the one or more intended nucleotide edits results in one or more codons that encode one or more amino acids different from the wild type clarin-1 protein but allows for expression of a functional clarin-1 protein.

A guide RNA core (also referred to herein as the gRNA core, gRNA scaffold, or gRNA backbone sequence) of a PEgRNA may contain a polynucleotide sequence that binds to a DNA binding domain (e.g., Cas9) of a prime editor. The gRNA core may interact with a prime editor as described herein, for example, by association with a DNA binding domain, such as a DNA nickase of the prime editor.

One of skill in the art will recognize that different prime editors having different DNA binding domains from different DNA binding proteins may require different gRNA core sequences specific to the DNA binding protein. In some embodiments, the gRNA core is capable of binding to a Cas9-based prime editor. In some embodiments, the gRNA core is capable of binding to a Cpf1-based prime editor. In some embodiments, the gRNA core is capable of binding to a Cas12b-based prime editor.

In some embodiments, the gRNA core comprises regions and secondary structures involved in binding with specific CRISPR Cas proteins. For example, in a Cas9 based prime editing system, the gRN,A core of a PEgRNA may comprise one or more regions of a base paired “lower stem” adjacent to the spacer sequence and a base paired “upper stem” following the lower stem, where the lower stem and upper stem may be connected by a “bulge” comprising unpaired RNAs. The gRNA core may further comprise a “nexus” distal from the spacer sequence, followed by a hairpin structure, e.g., at the 3′ end, as exemplified in FIG. 3. In some embodiments, the gRNA core comprises modified nucleotides as compared to a wild type gRNA core in the lower stem, upper stem, and/or the hairpin. For example, nucleotides in the lower stem, upper stem, an/or the hairpin regions may be modified, deleted, or replaced. In some embodiments, RNA nucleotides in the lower stem, upper stem, an/or the hairpin regions nay be rₑplaced with one or more DNA sequences. In some embodiments, the gRNA core comprises unmodified or wild type RNA sequences in the nexus and/or the bulge regions. In some embodiments, the gRNA core does not include long stretches of A-T pairs, for example, a GUUUU-AAAAC pairing element. In some embodiments, a prime editing system comprises a prime editor and a PEgRNA, wherein the prime editor comprises a SpCas9 nickase variant thereof, and the gRNA core of the PEgRNA comprises the sequence:

(SEQ ID NO: 665)
GUUUUAGAGCUAGAAAUAGCAAGUUAAAAUAAGGCUAGUCCGUUAUCAA
CUUGAAAAAGUGGCACCGAGUCGGUGC;
(SEQ ID NO: 666)
GUUUGAGAGCUAGAAAUAGCAAGUUUAAAUAAGGCUAGUCCGUUAUCAA
CUUGAAAAAGUGGGACCGAGUCGGUCC;
(SEQ ID NO: 667)
GUUUAAGAGCUAUGCUGGAAACAGCAUAGCAAGUUUAAAUAAGGCUAGU
CCGUUAUCAACUUGAAAAAGUGGCACCGAGUCGGUGC;
(SEQ ID NO: 668)
GUUUUAGUACUCUGGAAACAGAAUCUACUAAAACAAGGCAAAAUGCCGU
GUUUAUCUCGUCAACUUGUUGGCGAGA;
or
(SEQ ID NO: 669)
GUUUUAGUACUCUGGAAACAGAAUCUACUGAAACAAGACAAUAUGUCGU
GUUUAUCCCAUCAAUUUAUUGGUGGGA.

In some embodiments, the gRNA core comprises the sequence

(SEQ ID NO: 665)
GUUUUAGAGCUAGAAAUAGCAAGUUAAAAUAAGGCUAGUCCGUUAUCAA
CUUGAAAAAGUGGCACCGAGUCGGUGC.

Any gRNA core sequences known in the art are also contemplated in the prime editing compositions described herein.

A PEgRNA may also comprise optional modifiers, e.g., 3′ end modifier region and/or an 5′ end modifier region. In some embodiments, a PEgRNA comprises at least one nucleotide that is not part of a spacer, a gRNA core, or an extension arm. The optional sequence modifiers could be positioned within or between any of the other regions shown, and not limited to being located at the 3′ and 5′ ends. In certain embodiments, the PEgRNA comprises secondary RNA structure, such as, but not limited to, aptamers, hairpins, stem/loops, toeloops, and/or RNA-binding protein recruitment domains (e.g., the MS2 aptamer which recruits and binds to the MS2cp protein). In some embodiments, a PEgRNA comprises a short stretch of uracil at the 5′ end or the 3′ end. For example, in some embodiments, a PEgRNA comprising a 3′ extension arm comprises a “UUU” sequence at the 3′ end of the extension arm. In some embodiments, a PEgRNA comprises a toeloop sequence at the 3′ end. In some embodiments, the PEgRNA comprises a 3′ extension arm and a toeloop sequence at the 3′ end of the extension arm. In some embodiments, the PEgRNA comprises a 5′ extension arm and a toeloop sequence at the 5′ end of the extension arm. In some embodiments, the PEgRNA comprises a toeloop element having the sequence 5′-GAAANNNNN-3′, wherein N is any nucleobase. In some embodiments, the secondary RNA structure is positioned within the spacer. In some embodiments, the secondary structure is positioned within the extension arm. In some embodiments, the secondary structure is positioned within the gRNA core. In some embodiments, the secondary structure is positioned between the spacer and the gRNA core, between the gRNA core and the extension arm, or between the spacer and the extension arm. In some embodiments, the secondary structure is positioned between the PBS and the editing template. In some embodiments the secondary structure is positioned at the 3′ end or at the 5′ end of the PEgRNA. In some embodiments, the PEgRNA comprises a transcriptional termination signal at the 3′ end of the PEgRNA. In addition to secondary RNA structures, the PEgRNA may comprise a chemical linker or a poly(N) linker or tail, where “N” can be any nucleobase. In some embodiments, the chemical linker may function to prevent reverse transcription of the gRNA core.

In some embodiments, a prime editing system or composition further comprises a nick guide polynucleotide, such as a nick guide RNA (ngRNA). In some embodiments, a ngRNA comprises a spacer (referred to as a ngRNA spacer or ng spacer) and a gRNA core, wherein the spacer of the ngRNA comprises a region of complementarity to the edit strand, and wherein the gRNA core can interact with a Cas, e.g., Cas9, of a prime editor. Without wishing to be bound by any particular theory, an ngRNA may bind to the edit strand and direct the Cas nickase to generate a nick on the non-edit strand (or target strand). In some embodiments, the nick on the non-edit strand directs endogenous DNA repair machinery to use the edit strand as a template for repair of the non-edit strand, which may increase efficiency of prime editing. In some embodiments, the non-edit strand is nicked by a prime editor localized to the non-edit strand by the ngRNA. Accordingly, also provided herein are PEgRNA systems comprising at least one PEgRNA and at least one ngRNA.

A prime editing system comprising a PEgRNA (or one or more polynucleotide encoding the PEgRNA) and a prime editor protein (or one or more polynucleotides encoding the prime editor), may be referred to as a PE2 prime editing system and the corresponding editing approach referred to as PE2 approach or PE2 strategy. A PE2 system does not contain a ngRNA. A prime editing system comprising a PEgRNA (or one or more polynucleotide encoding the PEgRNA), a prime editor protein (or one or more polynucleotides encoding the prime editor), and a ngRNA (or one or more polynucleotides encoding the ngRNA) may be referred to as a “PE3” prime editing system. In some embodiments, an ngRNA spacer sequence is complementary to a portion of the edit strand that includes the intended nucleotide edit, and may hybridize with the edit strand only after the edit has been incorporated on the edit strand. Such ngRNA may be referred to a “PE3b” ngRNA, and the prime editing system a PE3b prime editing system.

In some embodiments, a PEgRNA or a nick guide RNA (ngRNA) may be chemically synthesized, or may be assembled or cloned and transcribed from a DNA sequence, e.g., a plasmid DNA sequence, or by any RNA oligonucleotide synthesis method known in the art. In some embodiments, DNA sequence that encodes a PEgRNA (or ngRNA) may be designed to append one or more nucleotides at the 5′ end or the 3′ end of the PEgRNA (or nick guide RNA) encoding sequence to enhance PEgRNA transcription. For example, in some embodiments, a DNA sequence that encodes a PEgRNA (or nick guide RNA) (or an ngRNA) may be designed to append a nucleotide G at the 5′ end. Accordingly, in some embodiments, the PEgRNA (or nick guide RNA) may comprise an appended nucleotide G at the 5′ end. In some embodiments, a DNA sequence that encodes a PEgRNA (or nick guide RNA) may be designed to append a sequence that enhances transcription, e.g., a Kozak sequence, at the 5′ end. In some embodiments, a DNA sequence that encodes a PEgRNA (or nick guide RNA) may be designed to append the sequence CACC or CCACC at the 5′ end. Accordingly, in some embodiments, the PEgRNA (or nick guide RNA) may comprise an appended sequence CACC or CCACC at the 5′ end. In some embodiments, a DNA sequence that encodes a PEgRNA (or nick guide RNA) may be designed to append the sequence TTT, TTTT, ITITT, TTFTT, TTTTTTT at the₃′ end. Accordingly, in some embodiments, the PEgRNA (or nick guide RNA) may comprise an appended sequence UUU, UUJUU, UUUU, UUUUUU, or UIJUUUU at the 3′ end.

In some embodiments, a prime editing system or composition further comprises a nick guide polynucleotide, such as a nick guide RNA (ngRNA). Without wishing to be bound by any particular theory, the non-edit strand of a double stranded target DNA in the target gene may be nicked by a CRISPR-Cas nickase directed by an ngRNA. In some embodiments, the nick on the non-edit strand directs endogenous DNA repair machinery to use the edit strand as a template for repair of the non-edit strand, which may increase efficiency of prime editing. In some embodiments, the non-edit strand is nicked by a prime editor localized to the non-edit strand by the ngRNA. Accordingly, also provided herein are PEgRNA systems comprising at least one PEgRNA and at least one ngRNA.

In some embodiments, a PEgRNA or ngRNA may include a modifying sequence at the 3′end having the sequence AACAUUGACGCGUCUCUACGUGGGGGCGCG (SEQ ID NO: 670).

In some embodiments, a PEgRNA or ngRNA comprises at the 3′ end a linker sequence comprising the sequence AACAUUGA (Sequence Number: 671).

In some embodiments, a PEgRNA or ngRNA comprises at the 3′ end a modifying sequence comprising the sequence CGCGUCUCUACGUGGGGGCGCG (SEQ ID NO: 672).

In some embodiments, the ngRNA is a guide RNA which contains a variable spacer sequence and a guide RNA scaffold or core region that interacts with the DNA binding domain, e.g., Cas9 of the prime editor. In some embodiments, the ngRNA comprises a spacer sequence (referred to herein as an ng spacer, or a second spacer) that is substantially complementary to a second search target sequence (or ng search target sequence), which is located on the edit strand, or the non-target strand. Thus, in some embodiments, the ng search target sequence recognized by the ng spacer and the search target sequence recognized by the spacer sequence of the PEgRNA are on opposite strands of the double stranded target DNA of target gene, e.g., the CLRN1 gene. A prime editing system, composition, or complex comprising a ngRNA may be referred to as a “PE3” prime editing system, PE3 prime editing composition, or PE3 prime editing complex.

In some embodiments, the ng search target sequence is located on the non-target strand, within 10 base pairs to 100 base pairs of an intended nucleotide edit incorporated by the PEgRNA on the edit strand. In some embodiments, the ng target search target sequence is within 10 bp, 20 bp, 30 bp, 40 bp, 50 bp, 60 bp, 70 bp, 80 bp, 90 bp, 91 bp, 92 bp, 93 bp, 94 bp, 95 bp, 96 bp, 97 bp, 98 bp, 99 bp, or 100 bp of an intended nucleotide edit incorporated by the PEgRNA on the edit strand. In some embodiments, the 5′ ends of the ng search target sequence and the PEgRNA search target sequence are within 1, 2, 3, 4, 5, 6, 7, 8, 9, or 10 bp apart from each other. In some embodiments, the 5′ ends of the ng search target sequence and the PEgRNA search target sequence are within 10 bp, 20 bp, 30 bp, 40 bp, 50 bp, 60 bp, 70 bp, 80 bp, 90 bp, 91 bp, 92 bp, 93 bp, 94 bp, 95 bp, 96 bp, 97 bp, 98 bp, 99 bp, or 100 bp apart from each other. In some embodiments, an ng spacer sequence is complementary to, and may hybridize with the second search target sequence only after an intended nucleotide edit has been incorporated on the edit strand, by the editing template of a PEgRNA. In some embodiments, such a prime editing system maybe referred to as a “PE3b” prime editing system or composition. In some embodiments, the ngRNA comprises a spacer sequence that matches only the edit strand after incorporation of the nucleotide edits, but not the endogenous target gene sequence on the edit strand. Accordingly, in some embodiments, an intended nucleotide edit is incorporated within the ng search target sequence.

A ngRNA protospacer may be in close proximity to the PEgRNA spacer, or may be upstream or downstream of the PEgRNA spacer. In some embodiments, the distance generated by the PEgRNA nick site and the ngRNA nick site (referred to as the nick-to-nick distance) is about 3 to about 100 nucleotides. In some embodiments, the distance generated by the PEgRNA nick site and the ngRNA nick site (referred to as the nick-to-nick distance) is about 4-90, 4-80, 4-70, 4-60, 4-50, 4-40, 4-30, 4-20, or 4-10 nucleotides. In some embodiments, the distance generated by the PEgRNA nick site and the ngRNA nick site (referred to as the nick-to-nick distance) is about 10-20, 20-30, 30-40, 40-50, 50-60, 60-70, 70-80,80-90, or 90-100 nucleotides. In some embodiments, the nick-to-nick distance is about 4-88 nucleotides. In some embodiments, the nick-to-nick distance is about 4-72 nucleotides. In some embodiments, the nick-to-nick distance is about 4-61 nucleotides. In some embodiments, the nick-to-nick distance is about 61-72 nucleotides. In some embodiments, the nick-to-nick distance is about 61-88 nucleotides. In some embodiments, the nick-to-nick distance is about 72-88 nucleotides. In some embodiments, the nick-to-nick distance is about 4-7 nucleotides. In some embodiments, the nick-to-nick distance is 4, 5, 6, or 7 nucleotides. In some embodiments, the nick-to-nick distance is about 41-96 nucleotides. In some embodiments, the nick-to-nick distance is about 41-82 nucleotides. In some embodiments, the nick-to-nick distance is about 41-44 nucleotides. In some embodiments, the nick-to-nick distance is about 44-82 nucleotides. In some embodiments, the nick-to-nick distance is about 44-96 nucleotides. In some embodiments, the nick-to-nick distance is about 82-96 nucleotides. In some embodiments, the nick-to-nick distance is 41, 44, 82, or 96 nucleotides. In some embodiments, the intended nucleotide edit is incorporated within about 1-10 nucleotides of the position corresponding to the PAM of the ng search target sequence.

The gRNA core of a PEgRNA or ngRNA can be any gRNA scaffold sequence that is capable of interacting with a Cas protein that recognizes the corresponding PAM of the PEgRNA or ngRNA. In some embodiments, gRNA core of a PEgRNA or a ngRNA comprises a sequence selected from SEQ ID Nos: 665-669.

A PEgRNA and/or an ngRNA of this disclosure, in some embodiments, may include modified nucleotides, e.g., chemically modified DNA or RNA nucleobases, and may include one or more nucleobase analogs (e.g., modifications which might add functionality, such as temperature resilience). In some embodiments, PEgRNAs and/or ngRNAs as described herein may be chemically modified. The phrase “chemical modifications,” as used herein, can include modifications which introduce chemistries which differ from those seen in naturally occurring DNA or RNAs, for example, covalent modifications such as the introduction of modified nucleotides, (e.g., nucleotide analogs, or the inclusion of pendant groups which are not naturally found in DNA or RNA molecules).

In some embodiments, the PEgRNAs provided in the disclosure may further comprise nucleotides added to the 5′ of the PEgRNAs. In some embodiments, the PEgRNA further comprises 1, 2, or 3 additional nucleotides added to the 5′ end. The additional nucleotides can be guanine, cytosine, adenine, or uracil. In some embodiments, the additional nucleotide at the 5′ end of the PEgRNA is a guanine or cytosine. In some embodiments, the additional nucleotides can be chemically or biologically modified.

In some embodiments, the PEgRNAs provided in the disclosure may further comprise nucleotides to the 3′ of the PEgRNAs. In some embodiments, the PEgRNA further comprises 1, 2, or 3 additional nucleotides to the 3′ end. The additional nucleotides can be guanine, cytosine, adenine, or uracil. In some embodiments, the additional nucleotides at the 3′ end of the PEgRNA is a polynucleotide comprising at least 1 uracil. In some embodiments, the additional nucleotides can be chemically or biologically modified.

In some embodiments, a PEgRNA or ngRNA is produced by transcription from a template nucleotide, for example, a template plasmid. In some embodiments, a polynucleotide encoding the PEgRNA or ngRNA is appended with one or more additional nucleotides that improves PEgRNA or ngRNA function or expression, e.g., expression from a plasmid that encodes the PEgRNA or ngRNA. In some embodiments, a polynucleotide encoding a PEgRNA or ngRNA is appended with one or more additional nucleotides at the 5′ end or at the 3′ end. In some embodiments, the polynucleotide encoding the PEgRNA or ngRNA is appended with a guanine at the 5′ end, for example, if the first nucleotide at the 5′ end of the spacer is not a guanine. In some embodiments, a polynucleotide encoding the PEgRNA or ngRNA is appended with nucleotide sequence CACC at the 5′ end. In some embodiments, the polynucleotide encoding the PEgRNA or ngRNA is appended with additional nucleotide sequence TTTTTT, TTTTTTT, TTTTT, or TTTT at the 3′ end. In some embodiments, the PEgRNA or ngRNA comprises the appended nucleotides from the transcription template. In some embodiments, the PEgRNA or ngRNA further comprises one or more nucleotides at the 5′ end or the 3′ end in addition to spacer, PBS, and RTT sequences, in some embodiments, the PEgRNA or ngRNA further comprises a guanine at the 5′ end, for example, when the first nucleotide at the 5′ end of the spacer is not a guanine. In some embodiments, the PEgRNA or ngRNA further comprises nucleotide sequence CACC at the 5′ end. In some embodiments, the PEgRNA or ngRNA further comprises an adenine at the 3′ end, for example, if the last nucleotide at the 3′ end of the PBS is a thymine. In some embodiments, the PEgRNA or ngRNA further comprises nucleotide sequence UUUUUUU, UUUUUU, UUUUU, or UUUU at the 3′ end.

In some embodiments, the PEgRNAs and/or ngRNAs provided in this disclosure may have undergone a chemical or biological modifications. Modifications may be made at any position within a PEgRNA or ngRNA, and may include modification to a nucleobase or to a phosphate backbone of the PEgRNA or ngRNA. In some embodiments, chemical modifications can be a structure guided modifications. In some embodiments, a chemical modification is at the 5′ end and/or the 3′ end of a PEgRNA. In some embodiments, a chemical modification is at the 5′ end and/or the 3′ end of a ngRNA. In some embodiments, a chemical modification may be within the spacer sequence, the extension arm, the editing template sequence, or the primer binding site of a PEgRNA. In some embodiments, a chemical modification may be within the spacer sequence or the gRNA core of a PEgRNA or a ngRNA. In some embodiments, a chemical modification may be within the 3′ most nucleotides of a PEgRNA or ngRNA. In some embodiments, a chemical modification may be within the 3′ most end of a PEgRNA or ngRNA. In some embodiments, the PEgRNA or ngRNA comprises 1, 2, 3, 4, 5, 6, 7, 8, 9, 10 or more chemically modified nucleotides at the 3′ end. In some embodiments, a PEgRNA or ngRNA comprises 3 contiguous chemically modified nucleotides at the 3′ end. In some embodiments, a chemical modification may be within the 5′ most end of a PEgRNA or ngRNA. In some embodiments, a PEgRNA or ngRNA comprises 1, 2, 3, 4, 5 or more chemically modified nucleotides at the 3′ end. In some embodiments, a PEgRNA or ngRNA comprises 1, 2, 3, 4, 5, 6, 7, 8, 9, 10 or more chemically modified nucleotides at the 5′ end. In some embodiments, a PEgRNA or ngRNA comprises 1, 2, 3, 4, 5, 6, 7, 8, 9, 10 or more chemically modified nucleotides at the 5′ end. In some embodiments, a PEgRNA or ngRNA comprises 1, 2, 3, 4, or 5 or more chemically modified nucleotides at the 3′ end. In some embodiments, a PEgRNA or ngRNA comprises 1, 2, 3, 4, or 5 more chemically modified nucleotides at the 5′ end. In some embodiments, a PEgRNA or ngRNA comprises 1, 2, or 3 or more chemically modified nucleotides at the 3′ end. In some embodiments, a PEgRNA or ngRNA comprises 1, 2, or 3 more chemically modified nucleotides at the 5′ end. In some embodiments, a PEgRNA or ngRNA comprises 1, 2, 3, 4, 5, 6, 7, 8, 9, 10 or more contiguous chemically modified nucleotides at the 3′ end. In some embodiments, a PEgRNA or ngRNA comprises 1, 2, 3, 4, 5, 6, 7, 8, 9, 10 or more contiguous chemically modified nucleotides at the 5′ end. In some embodiments, a PEgRNA or ngRNA comprises 1, 2, 3, 4, or 5 contiguous chemically modified nucleotides at the 3′ end. In some embodiments, a PEgRNA or ngRNA comprises 1, 2, 3, 4, or 5 contiguous chemically modified nucleotides at the 5′ end. In some embodiments, a PEgRNA or ngRNA comprises 1, 2, or 3 contiguous chemically modified nucleotides at the 3′ end. In some embodiments, a PEgRNA or ngRNA comprises 1, 2, or 3 contiguous chemically modified nucleotides at the 5′ end. In some embodiments, a PEgRNA or ngRNA comprises 3 contiguous chemically modified nucleotides at the 3′ end. In some embodiments, a PEgRNA or ngRNA comprises 1, 2, 3, 4, 5, or more chemically modified nucleotides near the 3′ end. In some embodiments, a PEgRNA or ngRNA comprises 3 contiguous chemically modified nucleotides at the 3′ end. In some embodiments, a PEgRNA or ngRNA comprises 3 contiguous chemically modified nucleotides at the 5′ end. In some embodiments, a PEgRNA or ngRNA comprises 1, 2, 3, 4, 5, or more chemically modified nucleotides near the 3′ end. In some embodiments, a PEgRNA or ngRNA comprises 1, 2, 3, 4, 5, or more contiguous chemically modified nucleotides near the 3′ end. In some embodiments, a PEgRNA or ngRNA comprises 1, 2, 3, 4, 5, or more chemically modified nucleotides near the 3′ end, where the 3′ most nucleotide is not modified, and the 1, 2, 3, 4, 5, or more chemically modified nucleotides precede the 3′ most nucleotide in a 5′-to-3′ order. In some embodiments, a PEgRNA or ngRNA comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more chemically modified nucleotides near the 3′ end, where the 3′ most nucleotide is not modified, and the 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more chemically modified nucleotides precede the 3′ most nucleotide in a 5′-to-3′ order.

In some embodiments, a PEgRNA or ngRNA comprises one or more chemical modified nucleotides in the gRNA core. As exemplified in FIG. 3, the gRNA core of a PEgRNA may comprise one or more regions of a base paired lower stem, a base paired upper stem, where the lower stem and upper stem may be connected by a bulge comprising unpaired RNAs. The gRNA core may firther comprise a nexus distal from the spacer sequence. In some embodiments, the gRNA core comprises one or more chemically modified nucleotides in the lower stem, upper stem, and/or the hairpin regions. In some embodiments, all of the nucleotides in the lower stem, upper stem, and/or the hairpin regions are chemically modified.

A chemical modification to a PEgRNA or ngRNA can comprise a 2′-O-thionocarbamate-protected nucleoside phosphoramidite, a 2′-O-methyl (M), a 2′-O-methyl 3′phosphorothioate (MS), or a 2′-O-methyl 3′thioPACE (MSP), or any combination thereof. In some embodiments, a chemically modified PEgRNA and/or ngRNA can comprise a 2′-O-methyl (M) RNA, a 2′-O-methyl 3′phosphorothioate (MS) RNA, a 2′-O-methyl 3′thioPACE (MSP) RNA, a 2′-F RNA, a phosphorothioate bond modification, any other chemical modifications known in the art, or any combination thereof. A chemical modification may also include, for example, the incorporation of non-nucleotide linkages or modified nucleotides into the PEgRNA and/or ngRNA (e.g., modifications to one or both of the 3′ and 5′ ends of a guide RNA molecule). Such modifications can include the addition of bases to an RNA sequence, complexing the RNA with an agent (e.g., a protein or a complementary nucleic acid molecule), and inclusion of elements which change the structure of an RNA molecule (e.g., which form secondary structures).

### Prime Editing Compositions

Disclosed herein, in some embodiments, are compositions, systems, and methods using a prime editing composition. The term “prime editing composition” or “prime editing system” refers to compositions involved in the method of prime editing as described herein. A prime editing composition may include a prime editor, e.g., a prime editor fusion protein, and a PEgRNA. A prime editing composition may further comprise additional elements, such as second strand nicking ngRNAs. Components of a prime editing composition may be combined to form a complex for prime editing, or may be kept separately, e.g., for administration purposes.

In some embodiments, a prime editing composition comprises a prime editor fusion protein complexed with a PEgRNA and optionally complexed with a ngRNA. In some embodiments, the prime editing composition comprises a prime editor comprising a DNA binding domain and a DNA polymerase domain associated with each other through a PEgRNA. For example, the prime editing composition may comprise a prime editor comprising a DNA binding domain and a DNA polymerase domain linked to each other by an RNA-protein recruitment aptamer RNA sequence, which is linked to a PEgRNA. In some embodiments, a prime editing composition comprises a PEgRNA and a polynucleotide, a polynucleotide construct, or a vector that encodes a prime editor fusion protein.

In some embodiments, a prime editing composition comprises a PEgRNA, a ngRNA, and a polynucleotide, a polynucleotide construct, or a vector that encodes a prime editor fusion protein. In some embodiments, a prime editing composition comprises multiple polynucleotides, polynucleotide constructs, or vectors, each of which encodes one or more prime editing composition components. In some embodiments, the PEgRNA of a prime editing composition is associated with the DNA binding domain, e.g., a Cas9 nickase, of the prime editor. In some embodiments, the PEgRNA of a prime editing composition complexes with the DNA binding domain of a prime editor and directs the prime editor to the target DNA.

In some embodiments, a prime editing composition comprises one or more polynucleotides that encode prime editor components and/or PEgRNA or ngRNAs. In some embodiments, a prime editing composition comprises a polynucleotide encoding a fusion protein comprising a DNA binding domain and a DNA polymerase domain. In some embodiments, a prime editing composition comprises (i) a polynucleotide encoding a fusion protein comprising a DNA binding domain and a DNA polymerase domain, and (ii) a PEgRNA or a polynucleotide encoding the PEgRNA. In some embodiments, a prime editing composition comprises (i) a polynucleotide encoding a fusion protein comprising a DNA binding domain and a DNA polymerase domain, (ii) a PEgRNA or a polynucleotide encoding the PEgRNA, and (iii) an ngRNA or a polynucleotide encoding the ngRNA. In some embodiments, a prime editing composition comprises (i) a polynucleotide encoding a DNA binding domain of a prime editor, e.g., a Cas9 nickase, (ii) a polynucleotide encoding a DNA polymerase domain of a prime editor, e.g., a reverse transcriptase, and (iii) a PEgRNA or a polynucleotide encoding the PEgRNA. In some embodiments, a prime editing composition comprises (i) a polynucleotide encoding a DNA binding domain of a prime editor, e.g., a Cas9 nickase, (ii) a polynucleotide encoding a DNA polymerase domain of a prime editor, e.g., a reverse transcriptase, (iii) a PEgRNA or a polynucleotide encoding the PEgRNA, and (iv) an ngRNA or a polynucleotide encoding the ngRNA.

In some embodiments, the polynucleotide encoding the DNA biding domain or the polynucleotide encoding the DNA polymerase domain further encodes an additional polypeptide domain, e.g., an RNA-protein recruitment domain, such as a MS2 coat protein domain. In some embodiments, a prime editing composition comprises (i) a polynucleotide encoding a N-terminal half of a prime editor fusion protein and an intein-N and (ii) a polynucleotide encoding a C-terminal half of a prime editor fusion protein and an intein-C. In some embodiments, a prime editing composition comprises (i) a polynucleotide encoding a N-terminal half of a prime editor fusion protein and an intein-N(ii) a polynucleotide encoding a C-terminal half of a prime editor fusion protein and an intein-C, (iii) a PEgRNA or a polynucleotide encoding the PEgRNA, and/or (iv) an ngRNA or a polynucleotide encoding the ngRNA. In some embodiments, a prime editing composition comprises (i) a polynucleotide encoding a N-terminal portion of a DNA binding domain and an intein-N, (ii) a polynucleotide encoding a C-terminal portion of the DNA binding domain, an intein-C, and a DNA polymerase domain. In some embodiments, the DNA binding domain is a Cas protein domain, e.g., a Cas9 nickase. In some embodiments, the prime editing composition comprises (i) a polynucleotide encoding a N-terminal portion of a DNA binding domain and an intein-N, (ii) a polynucleotide encoding a C-terminal portion of the DNA binding domain, an intein-C, and a DNA polymerase domain, (iii) a PEgRNA or a polynucleotide encoding the PEgRNA, and/or (iv) a ngRNA or a polynucleotide encoding the ngRNA.

In some embodiments, a prime editing system comprises one or more polynucleotides encoding one or more prime editor polypeptides, wherein activity of the prime editing system can be temporally regulated by controlling the timing in which the vectors are delivered. For example, in some embodiments, a polynucleotide encoding the prime editor and a polynucleotide encoding a PEgRNA can be delivered simultaneously. For example, in some embodiments, a polynucleotide encoding the prime editor and a polynucleotide encoding a PEgRNA may be delivered sequentially.

In some embodiments, a polynucleotide encoding a component of a prime editing system may further comprise an element that is capable of modifying the intracellular half-life of the polynucleotide and/or modulating translational control. In some embodiments, the polynucleotide is a RNA, for example, an mRNA. In some embodiments, the half-life of the polynucleotide, e.g., the RNA may be increased. In some embodiments, the half-life of the polynucleotide, e.g., the RNA may be decreased. In some embodiments, the element may be capable of increasing the stability of the polynucleotide, e.g., the RNA. In some embodiments, the element may be capable of decreasing the stability of the polynucleotide, e.g., the RNA. In some embodiments, the element may be within the 3′ UTR of the RNA. In some embodiments, the element may include a polyadenylation signal (PA). In some embodiments, the element may include a cap, e.g., an upstream mRNA or PEgRNA end. In some embodiments, the RNA may comprise no PA such that it is subject to quicker degradation in the cell after transcription.

In some embodiments, the element may include at least one AU-rich element (ARE). The AREs may be bound by ARE binding proteins (ARE-BPs) in a manner that is dependent upon tissue type, cell type, timing, cellular localization, and environment. In some embodiments the destabilizing element may promote RNA decay, affect RNA stability, or activate translation. In some embodiments, the AREs may comprise 50 to 150 nucleotides in length. In some embodiments, the AREs may comprise at least one copy of the sequence AUUUA. In some embodiments, at least one ARE may be added to the 3′ UTR of the RNA. In some embodiments, the element may be a Woodchuck Hepatitis Virus Posttranscriptional Regulatory Element (WPRE). In further embodiments, the element is a modified and/or truncated WPRE sequence that is capable of enhancing expression from the transcript. In some embodiments, the WPRE or equivalent may be added to the 3′ UTR of the RNA. In some embodiments, the element may be selected from other RNA sequence motifs that are enriched in either fast- or slow-decaying transcripts. In some embodiments, the polynucleotide, e.g., a vector, encoding the PE or the PEgRNA may be self-destroyed via cleavage of a target sequence present on the polynucleotide, e.g., a vector. The cleavage may prevent continued transcription of a PE or a PEgRNA.

Polynucleotides encoding prime editing composition components can be DNA, RNA, or any combination thereof. In some embodiments, a polynucleotide encoding a prime editing composition component is an expression construct. In some embodiments, a polynucleotide encoding a prime editing composition component is a vector. In some embodiments, the vector is a DNA vector. In some embodiments, the vector is a plasmid. In some embodiments, the vector is a virus vector, e.g., a retroviral vector, adenoviral vector, lentiviral vector, herpesvirus vector, or an adeno-associated virus vector (AAV).

In some embodiments, polynucleotides encoding polypeptide components of a prime editing composition are codon optimized by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. In some embodiments, a polynucleotide encoding a polypeptide component of a prime editing composition are operably linked to one or more expression regulatory elements, for example, a promoter, a 3′ UTR, a 5′ UTR, or any combination thereof. In some embodiments, a polynucleotide encoding a prime editing composition component is a messenger RNA (mRNA). In some embodiments, the mRNA comprises a Cap at the 5′ end and/or a poly A tail at the 3′ end.

Unless otherwise indicated, references to nucleotide positions in human chromosomes are as set forth in human genome assembly consortium Human build 38 (GRCh38), GenBank accession GCF_000001405.38.

Exemplary combinations of Prime Editing guide RNA (PEgRNA) components, e.g., spacer, PBS, and edit template/RTT, as well as combinations of each PEgRNA and corresponding ngRNA(s) are provided in Table 1. Table 1 contains three columns. The left column is the sequence number. The middle column provides the sequence of the component, labeled with a SEQ ID NO where allowed by the ST.26 standard. Although all the sequences provided in Table 1 are RNA sequences, “T” is used instead of a “U” in the sequences for consistency with the ST.26 standard. The right column contains a description of the sequence. All of the PEgRNAs in Table 1 are designed to correct a c. 144 T->G mutation in the Clrn1 gene; this mutation results in a N48K mutation in the encoded clarin 1 protein. However, the PEgRNA disclosed in Table 1 are also capable of correcting any other mutations in the Clrnl gene that are found in the portion of the gene that shares homology or complementarity with the edit template/RTT.

Table 1 provides Prime Editing guide RNAs (PEgRNAs) that can be used with any Prime Editor containing a Cas9 protein capable of recognizing an AGG PAM sequence. The PEgRNAs exemplified in Table 1 comprise: (a) a spacer comprising at its 3′ end a sequence corresponding to a listed PEgRNA spacer sequence; (b) a gRNA core capable of complexing with a Cas9 protein, and (c) an extension arm comprising: (i) an editing template comprising at its 3′ end any RTT sequence from Table 1, and (ii) a prime binding site (PBS) comprising at its 5′ end any PBS sequence from Table 1. The PEgRNA spacer can be, for example, 17-22 nucleotides in length. The PEgRNA spacers in Table 1 are annotated with their PAM sequence(s), enabling the selection of a prime editor comprising an appropriate Cas9 protein. The editing template can be referred to as a reverse transcription template (RTT). The editing template can encode wildtype CLRN1 gene sequence. Such editing templates are annotated as RTT in the description column of Table 1. Alternatively, the editing template can encode one or more mutations relative to the wildtype CLRN1 gene. The one or more mutations can include synonymous mutations, which preserve the wildtype amino acid sequence of the clarin 1 protein, and/or nonsynonymous mutations, which alter the amino acid sequence with respect to the wild type clarin 1 protein. RTT and pegRNA encoding synonymous mutations are annotated with the nucleotide changes in the description column of Table 1; RTT and pegRNA encoding nonsynonymous mutations are annotated with both the nucleotide and amino acid changes. The one or more mutations can include PAM silencing mutations, and are annotated as such in Table 1. In Table 1, some RTT are further annotated with a * followed by a number code. As described below, a PE3 or PE3b ngRNA spacers annotated with the same * and number code as an RTT has perfect complementarity to the edit strand post-edit by a PEgRNA containing the RTT. The PBS can be, for example, 5 to 19 nucleotides in length.

Specifically exemplified in Table 1 are pegRNA comprising (a) a spacer comprising at its 3′ end a sequence corresponding to sequence number 1; (b) a gRNA core capable of complexing with a Cas9 protein, and (c) an extension arm comprising: (i) an editing template comprising at its 3′ end a sequence corresponding to any one of sequence numbers 22-26, and (ii) a prime binding site (PBS) comprising at its 5′ end a sequence corresponding to sequence number 7. The PEgRNA spacer can be, for example, 17-22 nucleotides in length and can comprise the sequence corresponding to any one of sequence numbers 1-6. In some embodiments, the PEgRNA spacer comprises sequence number 4. The editing template can be referred to as a reverse transcription template (RTT). The editing template can encode wildtype CLRN1 gene sequence. For example, the editing template can comprise at its 3′ end the sequence corresponding to sequence number 23, 28, 35, 39, 44, 48, 54, 59, 63, 67, 71, 75, 79, 83, 87, 91, 95, 99,103,107, 111, 115, 119, 123, 127, 131, 135, 139, or 143. Alternatively, the editing template can encode one or more mutations relative to the wildtype CLRN1 gene. For example, the editing template can encode an AGG-to-ATG synonymous PAM silencing mutation and comprise at its 3′ end the sequence corresponding to sequence number 22, 27, 34, 38, 43, 47, 53, 58, 62, 66, 70, 74, 78, 82, 86, 90, 94, 98,102,106, 110, 114, 118, 122, 126, 130, 134, 138, or 142. In another example, the editing template can encode an AGG-to-ACG synonymous PAM silencing mutation and comprise at its 3′ end the sequence corresponding to sequence number 24, 29, 36, 40, 45, 49, 55, 60, 64, 68, 72, 76, 80, 84, 88, 92, 96,100,104, 108, 112, 116, 120, 124, 128, 132, 136, 140, or 144. In another example, the editing template can encode an AGG-to-AAG synonymous PAM silencing mutation and comprise at its 3′ end the sequence corresponding to sequence number 25, 30, 37, 41, 46, 50, 56, 61, 65, 69, 73, 77, 81, 85, 89, 93, 97,101,105, 109, 113, 117, 121, 125, 129, 133, 137, 141, 145. In another example, the editing template can encode an AGG-to-AGC nonsynonymous [A49G] PAM silencing mutation and comprise at its 3′ end the sequence corresponding to sequence number 26, 33, 42, 51, or 57. The PBS can be, for example, 5 to 19 nucleotides in length and can comprise the sequence corresponding to any one of sequence numbers 7-21.

Any of the PEgRNA exemplified in Table 1 can comprise, from 5′ to 3′, the spacer, the gRNA core, the edit template, and the PBS. The 3′ end of the edit template can be contiguous with the 5′ end of the PBS. The PEgRNA can comprise multiple RNA molecules (e.g., a crRNA containing the PEgRNA spacer and a tracrRNA comprising the extension arm) or can be a single gRNA molecule comprising the extension arm. Exemplary PEgRNAs provided in Table 1 can comprise a sequence corresponding to any one of sequence numbers 195-508. Any PEgRNA exemplified in Table 1 may comprise, or further comprise, a 3′ motif at the 3′ end of the extension arm, for example, a linker (e.g., the linker of sequence number 671) and a hairpin-forming motif (e.g., the hairpin of SEQ ID NO: 672) or a series of 1, 2, 3, 4, 5, 6, 7 or more U nucleotides. In some embodiments, the PEgRNA comprises 4 U nucleotides at its 3′ end. Without being bound by theory, such 3′ motifs are believed to increase PEgRNA stability. The PEgRNA may alternatively or additionally comprise one or more chemical modifications, such as phosphorothioate (PS) bond(s), 2′-O-methylated (2′-Ome) nucleotides, or a combination thereof. In some embodiments, the PEgRNA comprise 3′ mN*mN*mN*N and 5′mN*mN*mN* modifications, where m indicates that the nucleotide contains a 2′-O-Me modification and a * indicates the presence of a phosphorothioate bond. PEgRNA sequences exemplified in Table 1 may alternatively be adapted for expression from a U6 promoter, for example, by including a 5′ terminal G if the spacer of the PEgRNA begins with another nucleotide, by including 6 or 7 U nucleotides at the 3′ end of the extension arm, or both. Those of skill in the art will recognize that transcription from a U6 promoter will result in a variable number of Us (e.g., 1-5 Us) actually being incorporated into the transcribed pegRNA sequence and any transcription adapted sequences are meant to encompass this biological variability. Such transcription-adapted sequences may further comprise a linker and hairpin-forming motif between the PBS and the 3′ terminal U series.

Any of the PEgRNAs exemplified in Table 1 can be used in a Prime Editing system further comprising a nick guide RNA (ngRNA). Such ngRNA can comprise a spacer comprising at its 3′ end a sequence corresponding to nucleotides 4-20 of any ngRNA spacer listed in the Table 1 and a gRNA core capable of complexing with a Cas9 protein. For example, the sequence in the spacer of the ngRNA can comprise nucleotides 4-20, 3-20, 2-20, or 1-20 of any one of sequence numbers 146-194. In some embodiments, the spacer of the ngRNA is the complete sequence of any one of sequence numbers 146-194. The ngRNA spacers in Table 1 are annotated with their PAM sequences, enabling selection of an appropriate Cas9 protein. It can be advantageous to select an ngRNA spacer that has a PAM sequence compatible with the Cas9 protein used in the Prime Editor with the PEgRNA, thus avoiding the need to use two different Cas9 proteins. The ngRNA can comprise multiple RNA molecules (e.g., a crRNA containing the ngRNA spacer and a tracrRNA) or can be a single gRNA molecule. The ngRNA is capable of directing a complexed Cas9 protein to bind the edit strand of the CLRN1 gene; thus, a complexed Cas9 nickase containing a nuclease inactivating mutation in the HNH domain will nick the non-edit strand. A PE3 ngRNA spacer has perfect complementarity to the edit strand both pre- and post-edit; a PE3b ngRNA spacer has perfect complementarity to the edit strand post-edit. A PE3 or PE3b spacer annotated with a * followed by a number code has perfect complementarity to the edit strand post-edit with a PEgRNA containing an RTT annotated with the same number code.

Exemplary ngRNAs provided in Table 1 can comprise a sequence corresponding to any one of sequence numbers 509-588. Any ngRNA exemplified in Table 1 may comprise, or further comprise, a series of 1, 2, 3, 4, 5, 6, 7 or more U nucleotides. In some embodiments, the ngRNA comprises 4 U nucleotides at its 3′ end. Without being bound by theory, such 3′ motifs are believed to increase ngRNA stability. The ngRNA may alternatively or additionally comprise one or more chemical modifications, such as phosphorothioate (PS) bond(s), 2′-O-methylated (2′-Ome) nucleotides, or a combination thereof. In some embodiments, the ngRNA comprise 3′ mN*mN*mN*N and 5′mN*mN*mN* modifications, where m indicates that the nucleotide contains a 2′-O-Me modification and a * indicates the presence of a phosphorothioate bond. NgRNA sequences may alternatively be adapted for expression from a DNA template, for example, by including a 5′ terminal G if the spacer of the ngRNA begins with another nucleotide, by including 6 or 7 U nucleotides at the 3′ end of the ngRNA, or both.

In some embodiments, the gRNA core for the PEgRNA and/or the ngRNA comprises a sequence selected from any one of SEQ ID NOs: 665-669. In some embodiments, the gRNA core comprises SEQ ID NO: 665.

TABLE 1

| Sequence | | |
|---|---|---|
| Number | Sequence | Description |
| 1 | GTCCAGCTCCTGCCCTG (SEQ ID NO: 1) | PEgRNA spacer (AGG PAM) |
| 2 | TGTCCAGCTCCTGCCCTG (SEQ ID NO: 2) | PEgRNA spacer (AGG PAM) |
| 3 | TTGTCCAGCTCCTGCCCTG (SEQ ID NO: 3) | PEgRNA spacer (AGG PAM) |
| 4 | CTTGTCCAGCTCCTGCCCTG (SEQ ID NO: 4) | PEgRNA spacer (AGG PAM) |
| 5 | ACTTGTCCAGCTCCTGCCCTG (SEQ ID NO: 5) | PEgRNA spacer (AGG PAM) |
| 6 | AACTTGTCCAGCTCCTGCCCTG (SEQ ID NO: 6) | PEgRNA spacer (AGG PAM) |
| 7 | GGCAG | PBS |
| 8 | GGCAGG | PBS |
| 9 | GGCAGGA | PBS |
| 10 | GGCAGGAG | PBS |
| 11 | GGCAGGAGC | PBS |
| 12 | GGCAGGAGCT (SEQ ID NO: 12) | PBS |
| 13 | GGCAGGAGCTG (SEQ ID NO: 13) | PBS |
| 14 | GGCAGGAGCTGG (SEQ ID NO: 14) | PBS |
| 15 | GGCAGGAGCTGGA (SEQ ID NO: 15) | PBS |
| 16 | GGCAGGAGCTGGAC (SEQ ID NO: 16) | PBS |
| 17 | GGCAGGAGCTGGACA (SEQ ID NO: 17) | PBS |
| 18 | GGCAGGAGCTGGACAA (SEQ ID NO: 18) | PBS |
| 19 | GGCAGGAGCTGGACAAG (SEQ ID NO: 19) | PBS |
| 20 | GGCAGGAGCTGGACAAGT (SEQ ID NO: 20) | PBS |
| 21 | GGCAGGAGCTGGACAAGTT (SEQ ID NO: 21) | PBS |
| 22 | TCAATGCATCAG (SEQ ID NO: 22) | RTT*1, 5, 9 (AGG-to-ATG PAM |
| | | silencing edit) |
| 23 | TCAATGCCTCAG (SEQ ID NO: 23) | RTT*2, 6, 10, 13 |
| 24 | TCAATGCGTCAG (SEQ ID NO: 24) | RTT*3, 7, 11 (AGG-to-ACG |
| | | PAM silencing edit) |
| 25 | TCAATGCTTCAG (SEQ ID NO: 25) | RTT*4, 8, 12 (AGG-to-AAG |
| | | PAM silencing edit) |
| 26 | TCAATGGCTCAG (SEQ ID NO: 26) | RTT*14, 15 (AGG-to-AGC |
| | | nonsynonymous [A49G] PAM |
| | | silencing edit) |
| 27 | GTCAATGCATCAG (SEQ ID NO: 27) | RTT*1, 5, 9 (AGG-to-ATG PAM |
| | | silencing edit) |
| 28 | GTCAATGCCTCAG (SEQ ID NO: 28) | RTT*2, 6, 10, 13 |
| 29 | GTCAATGCGTCAG (SEQ ID NO: 29) | RTT*3, 7, 11 (AGG-to-ACG |
| | | PAM silencing edit) |
| 30 | GTCAATGCTTCAG (SEQ ID NO: 30) | RTT*4, 8, 12 (AGG-to-AAG |
| | | PAM silencing edit) |
| 31 | TTCAATGCCTCAG (SEQ ID NO: 31) | RTT*13 (G-to-T |
| | | nonsynonymous [V47F] edit) |
| 32 | TTCAATGGCTCAG (SEQ ID NO: 32) | RTT (G-to-T nonsynonymous |
| | | [V47F]; AGG-to-AGC |
| | | nonsynonymous [A49G] PAM |
| | | silencing edit) |
| 33 | GTCAATGGCTCAG (SEQ ID NO: 33) | RTT*14, 15 (AGG-to-AGC |
| | | nonsynonymous [A49G] PAM |
| | | silencing edit) |
| 34 | CGTCAATGCATCAG (SEQ ID NO: 34) | RTT*1, 5, 9 (AGG-to-ATG PAM |
| | | silencing edit) |
| 35 | CGTCAATGCCTCAG (SEQ ID NO: 35) | RTT*2, 6, 10, 13 |
| 36 | CGTCAATGCGTCAG (SEQ ID NO: 36) | RTT*3, 7, 11 (AGG-to-ACG |
| | | PAM silencing edit) |
| 37 | CGTCAATGCTTCAG (SEQ ID NO: 37) | RTT*4, 8, 12 (AGG-to-AAG |
| | | PAM silencing edit) |
| 38 | TCGTCAATGCATCAG (SEQ ID NO: 38) | RTT*1, 5, 9 (AGG-to-ATG PAM |
| | | silencing edit) |
| 39 | TCGTCAATGCCTCAG (SEQ ID NO: 39) | RTT*2, 6, 10, 13 |
| 40 | TCGTCAATGCGTCAG (SEQ ID NO: 40) | RTT*3, 7, 11 (AGG-to-ACG |
| | | PAM silencing edit) |
| 41 | TCGTCAATGCTTCAG (SEQ ID NO: 41) | RTT*4, 8, 12 (AGG-to-AAG |
| | | PAM silencing edit) |
| 42 | TCGTCAATGGCTCAG (SEQ ID NO: 42) | RTT*14, 15 (AGG-to-AGC |
| | | nonsynonymous [A49G] PAM |
| | | silencing edit) |
| 43 | CTCGTCAATGCATCAG (SEQ ID NO: 43) | RTT*1, 5, 9 (AGG-to-ATG PAM |
| | | silencing edit) |
| 44 | CTCGTCAATGCCTCAG (SEQ ID NO: 44) | RTT*2, 6, 10, 13 |
| 45 | CTCGTCAATGCGTCAG (SEQ ID NO: 45) | RTT*3, 7, 11 (AGG-to-ACG |
| | | PAM silencing edit) |
| 46 | CTCGTCAATGCTTCAG (SEQ ID NO: 46) | RTT*4, 8, 12 (AGG-to-AAG |
| | | PAM silencing edit) |
| 47 | GCTCGTCAATGCATCAG (SEQ ID NO: 47) | RTT*1, 5, 9 (AGG-to-ATG PAM |
| | | silencing edit) |
| 48 | GCTCGTCAATGCCTCAG (SEQ ID NO: 48) | RTT*2, 6, 10, 13 |
| 49 | GCTCGTCAATGCGTCAG (SEQ ID NO: 49) | RTT*3, 7, 11 (AGG-to-ACG |
| | | PAM silencing edit) |
| 50 | GCTCGTCAATGCTTCAG (SEQ ID NO: 50) | RTT*4, 8, 12 (AGG-to-AAG |
| | | PAM silencing edit) |
| 51 | GCTCGTCAATGGCTCAG (SEQ ID NO: 51) | RTT*14, 15 (AGG-to-AGC |
| | | nonsynonymous [A49G] PAM |
| | | silencing edit) |
| 52 | TCTCGTCAATGGCTCAG (SEQ ID NO: 52) | RTT (G-to-T synonymous; |
| | | AGG-to-AGC nonsynonymous |
| | | [A49G] PAM silencing edit) |
| 53 | TGCTCGTCAATGCATCAG (SEQ ID NO: 53) | RTT*1, 5, 9 (AGG-to-ATG PAM |
| | | silencing edit) |
| 54 | TGCTCGTCAATGCCTCAG (SEQ ID NO: 54) | RTT*2, 6, 10, 13 |
| 55 | TGCTCGTCAATGCGTCAG (SEQ ID NO: 55) | RTT*3, 7, 11 (AGG-to-ACG |
| | | PAM silencing edit) |
| 56 | TGCTCGTCAATGCTTCAG (SEQ ID NO: 56) | RTT*4, 8, 12 (AGG-to-AAG |
| | | PAM silencing edit) |
| 57 | TGCTCGTCAATGGCTCAG (SEQ ID NO: 57) | RTT*14, 15 (AGG-to-AGC |
| | | nonsynonymous [A49G] PAM |
| | | silencing edit) |
| 58 | CTGCTCGTCAATGCATCAG (SEQ ID NO: 58) | RTT*1, 5, 9 (AGG-to-ATG PAM |
| | | silencing edit) |
| 59 | CTGCTCGTCAATGCCTCAG (SEQ ID NO: 59) | RTT*2, 6, 10, 13 |
| 60 | CTGCTCGTCAATGCGTCAG (SEQ ID NO: 60) | RTT*3, 7, 11 (AGG-to-ACG |
| | | PAM silencing edit) |
| 61 | CTGCTCGTCAATGCTTCAG (SEQ ID NO: 61) | RTT*4, 8, 12 (AGG-to-AAG |
| | | PAM silencing edit) |
| 62 | TCTGCTCGTCAATGCATCAG (SEQ ID NO: 62) | RTT*1, 5, 9 (AGG-to-ATG PAM |
| | | silencing edit) |
| 63 | TCTGCTCGTCAATGCCTCAG (SEQ ID NO: 63) | RTT*2, 6, 10, 13 |
| 64 | TCTGCTCGTCAATGCGTCAG (SEQ ID NO: 64) | RTT*3, 7, 11 (AGG-to-ACG |
| | | PAM silencing edit) |
| 65 | TCTGCTCGTCAATGCTTCAG (SEQ ID NO: 65) | RTT*4, 8, 12 (AGG-to-AAG |
| | | PAM silencing edit) |
| 66 | CTCTGCTCGTCAATGCATCAG (SEQ ID NO: 66) | RTT*1, 5, 9 (AGG-to-ATG PAM |
| | | silencing edit) |
| 67 | CTCTGCTCGTCAATGCCTCAG (SEQ ID NO: 67) | RTT*2, 6, 10, 13 |
| 68 | CTCTGCTCGTCAATGCGTCAG (SEQ ID NO: 68) | RTT*3, 7, 11 (AGG-to-ACG |
| | | PAM silencing edit) |
| 69 | CTCTGCTCGTCAATGCTTCAG (SEQ ID NO: 69) | RTT*4, 8, 12 (AGG-to-AAG |
| | | PAM silencing edit) |
| 70 | GCTCTGCTCGTCAATGCATCAG (SEQ ID NO: | RTT*1, 5, 9 (AGG-to-ATG PAM |
| | 70) | silencing edit) |
| 71 | GCTCTGCTCGTCAATGCCTCAG (SEQ ID NO: | RTT*2, 6, 10, 13 |
| | 71) | |
| 72 | GCTCTGCTCGTCAATGCGTCAG (SEQ ID NO: | RTT*3, 7, 11 (AGG-to-ACG |
| | 72) | PAM silencing edit) |
| 73 | GCTCTGCTCGTCAATGCTTCAG (SEQ ID NO: | RTT*4, 8, 12 (AGG-to-AAG |
| | 73) | PAM silencing edit) |
| 74 | AGCTCTGCTCGTCAATGCATCAG (SEQ ID NO: | RTT*1, 5, 9 (AGG-to-ATG PAM |
| | 74) | silencing edit) |
| 75 | AGCTCTGCTCGTCAATGCCTCAG (SEQ ID NO: | RTT*2, 6, 10, 13 |
| | 75) | |
| 76 | AGCTCTGCTCGTCAATGCGTCAG (SEQ ID NO: | RTT*3, 7, 11 (AGG-to-ACG |
| | 76) | PAM silencing edit) |
| 77 | AGCTCTGCTCGTCAATGCTTCAG (SEQ ID NO: | RTT*4, 8, 12 (AGG-to-AAG |
| | 77) | PAM silencing edit) |
| 78 | GAGCTCTGCTCGTCAATGCATCAG (SEQ ID NO: | RTT*1, 5, 9 (AGG-to-ATG PAM |
| | 78) | silencing edit) |
| 79 | GAGCTCTGCTCGTCAATGCCTCAG (SEQ ID NO: | RTT*2, 6, 10, 13 |
| | 79) | |
| 80 | GAGCTCTGCTCGTCAATGCGTCAG (SEQ ID NO: | RTT*3, 7, 11 (AGG-to-ACG |
| | 80) | PAM silencing edit) |
| 81 | GAGCTCTGCTCGTCAATGCTTCAG (SEQ ID NO: | RTT*4, 8, 12 (AGG-to-AAG |
| | 81) | PAM silencing edit) |
| 82 | GGAGCTCTGCTCGTCAATGCATCAG (SEQ ID | RTT*1, 5, 9 (AGG-to-ATG PAM |
| | NO: 82) | silencing edit) |
| 83 | GGAGCTCTGCTCGTCAATGCCTCAG (SEQ ID | RTT*2, 6, 10, 13 |
| | NO: 83) | |
| 84 | GGAGCTCTGCTCGTCAATGCGTCAG (SEQ ID | RTT*3, 7, 11 (AGG-to-ACG |
| | NO: 84) | PAM silencing edit) |
| 85 | GGAGCTCTGCTCGTCAATGCTTCAG (SEQ ID | RTT*4, 8, 12 (AGG-to-AAG |
| | NO: 85) | PAM silencing edit) |
| 86 | GGGAGCTCTGCTCGTCAATGCATCAG (SEQ ID | RTT*1, 5, 9 (AGG-to-ATG PAM |
| | NO: 86) | silencing edit) |
| 87 | GGGAGCTCTGCTCGTCAATGCCTCAG (SEQ ID | RTT*2, 6, 10, 13 |
| | NO: 87) | |
| 88 | GGGAGCTCTGCTCGTCAATGCGTCAG (SEQ ID | RTT*3, 7, 11 (AGG-to-ACG |
| | NO: 88) | PAM silencing edit) |
| 89 | GGGAGCTCTGCTCGTCAATGCTTCAG (SEQ ID | RTT*4, 8, 12 (AGG-to-AAG |
| | NO: 89) | PAM silencing edit) |
| 90 | CGGGAGCTCTGCTCGTCAATGCATCAG (SEQ | RTT*1, 5, 9 (AGG-to-ATG PAM |
| | ID NO: 90) | silencing edit) |
| 91 | CGGGAGCTCTGCTCGTCAATGCCTCAG (SEQ | RTT*2, 6, 10, 13 |
| | ID NO: 91) | |
| 92 | CGGGAGCTCTGCTCGTCAATGCGTCAG (SEQ | RTT*3, 7, 11 (AGG-to-ACG |
| | ID NO: 92) | PAM silencing edit) |
| 93 | CGGGAGCTCTGCTCGTCAATGCTTCAG (SEQ | RTT*4, 8, 12 (AGG-to-AAG |
| | ID NO: 93) | PAM silencing edit) |
| 94 | ACGGGAGCTCTGCTCGTCAATGCATCAG (SEQ | RTT*1, 5, 9 (AGG-to-ATG PAM |
| | ID NO: 94) | silencing edit) |
| 95 | ACGGGAGCTCTGCTCGTCAATGCCTCAG (SEQ | RTT*2, 6, 10, 13 |
| | ID NO: 95) | |
| 96 | ACGGGAGCTCTGCTCGTCAATGCGTCAG (SEQ | RTT*3, 7, 11 (AGG-to-ACG |
| | ID NO: 96) | PAM silencing edit) |
| 97 | ACGGGAGCTCTGCTCGTCAATGCTTCAG (SEQ | RTT*4, 8, 12 (AGG-to-AAG |
| | ID NO: 97) | PAM silencing edit) |
| 98 | AACGGGAGCTCTGCTCGTCAATGCATCAG | RTT*1, 5, 9 (AGG-to-ATG PAM |
| | (SEQ ID NO: 98) | silencing edit) |
| 99 | AACGGGAGCTCTGCTCGTCAATGCCTCAG | RTT*2, 6, 10, 13 |
| | (SEQ ID NO: 99) | |
| 100 | AACGGGAGCTCTGCTCGTCAATGCGTCAG | RTT*3, 7, 11 (AGG-to-ACG |
| | (SEQ ID NO: 100) | PAM silencing edit) |
| 101 | AACGGGAGCTCTGCTCGTCAATGCTTCAG | RTT*4, 8, 12 (AGG-to-AAG |
| | (SEQ ID NO: 101) | PAM silencing edit) |
| 102 | AAACGGGAGCTCTGCTCGTCAATGCATCAG | RTT*1, 5, 9 (AGG-to-ATG PAM |
| | (SEQ ID NO: 102) | silencing edit) |
| 103 | AAACGGGAGCTCTGCTCGTCAATGCCTCAG | RTT*2, 6, 10, 13 |
| | (SEQ ID NO: 103) | |
| 104 | AAACGGGAGCTCTGCTCGTCAATGCGTCAG | RTT*3, 7, 11 (AGG-to-ACG |
| | (SEQ ID NO: 104) | PAM silencing edit) |
| 105 | AAACGGGAGCTCTGCTCGTCAATGCTTCAG | RTT*4, 8, 12 (AGG-to-AAG |
| | (SEQ ID NO: 105) | PAM silencing edit) |
| 106 | AAAACGGGAGCTCTGCTCGTCAATGCATCAG | RTT*1, 5, 9 (AGG-to-ATG PAM |
| | (SEQ ID NO: 106) | silencing edit) |
| 107 | AAAACGGGAGCTCTGCTCGTCAATGCCTCAG | RTT*2, 6, 10, 13 |
| | (SEQ ID NO: 107) | |
| 108 | AAAACGGGAGCTCTGCTCGTCAATGCGTCAG | RTT*3, 7, 11 (AGG-to-ACG |
| | (SEQ ID NO: 108) | PAM silencing edit) |
| 109 | AAAACGGGAGCTCTGCTCGTCAATGCTTCAG | RTT*4, 8, 12 (AGG-to-AAG |
| | (SEQ ID NO: 109) | PAM silencing edit) |
| 110 | CAAAACGGGAGCTCTGCTCGTCAATGCATCAG | RTT*1, 5, 9 (AGG-to-ATG PAM |
| | (SEQ ID NO: 110) | silencing edit) |
| 111 | CAAAACGGGAGCTCTGCTCGTCAATGCCTCAG | RTT*2, 6, 10, 13 |
| | (SEQ ID NO: 111) | |
| 112 | CAAAACGGGAGCTCTGCTCGTCAATGCGTCAG | RTT*3, 7, 11 (AGG-to-ACG |
| | (SEQ ID NO: 112) | PAM silencing edit) |
| 113 | CAAAACGGGAGCTCTGCTCGTCAATGCTTCAG | RTT*4, 8, 12 (AGG-to-AAG |
| | (SEQ ID NO: 113) | PAM silencing edit) |
| 114 | GCAAAACGGGAGCTCTGCTCGTCAATGCATCA | RTT*1, 5, 9 (AGG-to-ATG PAM |
| | G (SEQ ID NO: 114) | silencing edit) |
| 115 | GCAAAACGGGAGCTCTGCTCGTCAATGCCTCA | RTT*2, 6, 10, 13 |
| | G (SEQ ID NO: 115) | |
| 116 | GCAAAACGGGAGCTCTGCTCGTCAATGCGTCA | RTT*3, 7, 11 (AGG-to-ACG |
| | G (SEQ ID NO: 116) | PAM silencing edit) |
| 117 | GCAAAACGGGAGCTCTGCTCGTCAATGCTTCA | RTT*4, 8, 12 (AGG-to-AAG |
| | G (SEQ ID NO: 117) | PAM silencing edit) |
| 118 | TGCAAAACGGGAGCTCTGCTCGTCAATGCATC | RTT*1, 5, 9 (AGG-to-ATG PAM |
| | AG (SEQ ID NO: 118) | silencing edit) |
| 119 | TGCAAAACGGGAGCTCTGCTCGTCAATGCCTC | RTT*2, 6, 10, 13 |
| | AG (SEQ ID NO: 119) | |
| 120 | TGCAAAACGGGAGCTCTGCTCGTCAATGCGTC | RTT*3, 7, 11 (AGG-to-ACG |
| | AG (SEQ ID NO: 120) | PAM silencing edit) |
| 121 | TGCAAAACGGGAGCTCTGCTCGTCAATGCTTC | RTT*4, 8, 12 (AGG-to-AAG |
| | AG (SEQ ID NO: 121) | PAM silencing edit) |
| 122 | CTGCAAAACGGGAGCTCTGCTCGTCAATGCAT | RTT*1, 5, 9 (AGG-to-ATG PAM |
| | CAG (SEQ ID NO: 122) | silencing edit) |
| 123 | CTGCAAAACGGGAGCTCTGCTCGTCAATGCCT | RTT*2, 6, 10, 13 |
| | CAG (SEQ ID NO: 123) | |
| 124 | CTGCAAAACGGGAGCTCTGCTCGTCAATGCGT | RTT*3, 7, 11 (AGG-to-ACG |
| | CAG (SEQ ID NO: 124) | PAM silencing edit) |
| 125 | CTGCAAAACGGGAGCTCTGCTCGTCAATGCTT | RTT*4, 8, 12 (AGG-to-AAG |
| | CAG (SEQ ID NO: 125) | PAM silencing edit) |
| 126 | TCTGCAAAACGGGAGCTCTGCTCGTCAATGCA | RTT*1, 5, 9 (AGG-to-ATG PAM |
| | TCAG (SEQ ID NO: 126) | silencing edit) |
| 127 | TCTGCAAAACGGGAGCTCTGCTCGTCAATGCC | RTT*2, 6, 10, 13 |
| | TCAG (SEQ ID NO: 127) | |
| 128 | TCTGCAAAACGGGAGCTCTGCTCGTCAATGCG | RTT*3, 7, 11 (AGG-to-ACG |
| | TCAG (SEQ ID NO: 128) | PAM silencing edit) |
| 129 | TCTGCAAAACGGGAGCTCTGCTCGTCAATGCT | RTT*4, 8, 12 (AGG-to-AAG |
| | TCAG (SEQ ID NO: 129) | PAM silencing edit) |
| 130 | CTCTGCAAAACGGGAGCTCTGCTCGTCAATGC | RTT*1, 5, 9 (AGG-to-ATG PAM |
| | ATCAG (SEQ ID NO: 130) | silencing edit) |
| 131 | CTCTGCAAAACGGGAGCTCTGCTCGTCAATGC | RTT*2, 6, 10, 13 |
| | CTCAG (SEQ ID NO: 131) | |
| 132 | CTCTGCAAAACGGGAGCTCTGCTCGTCAATGC | RTT*3, 7, 11 (AGG-to-ACG |
| | GTCAG (SEQ ID NO: 132) | PAM silencing edit) |
| 133 | CTCTGCAAAACGGGAGCTCTGCTCGTCAATGC | RTT*4, 8, 12 (AGG-to-AAG |
| | TTCAG (SEQ ID NO: 133) | PAM silencing edit) |
| 134 | CCTCTGCAAAACGGGAGCTCTGCTCGTCAATG | RTT*1, 5, 9 (AGG-to-ATG PAM |
| | CATCAG (SEQ ID NO: 134) | silencing edit) |
| 135 | CCTCTGCAAAACGGGAGCTCTGCTCGTCAATG | RTT*2, 6, 10, 13 |
| | CCTCAG (SEQ ID NO: 135) | |
| 136 | CCTCTGCAAAACGGGAGCTCTGCTCGTCAATG | RTT*3, 7, 11 (AGG-to-ACG |
| | CGTCAG (SEQ ID NO: 136) | PAM silencing edit) |
| 137 | CCTCTGCAAAACGGGAGCTCTGCTCGTCAATG | RTT*4, 8, 12 (AGG-to-AAG |
| | CTTCAG (SEQ ID NO: 137) | PAM silencing edit) |
| 138 | TCCTCTGCAAAACGGGAGCTCTGCTCGTCAAT | RTT*1, 5, 9 (AGG-to-ATG PAM |
| | GCATCAG (SEQ ID NO: 138) | silencing edit) |
| 139 | TCCTCTGCAAAACGGGAGCTCTGCTCGTCAAT | RTT*2, 6, 10, 13 |
| | GCCTCAG (SEQ ID NO: 139) | |
| 140 | TCCTCTGCAAAACGGGAGCTCTGCTCGTCAAT | RTT*3, 7, 11 (AGG-to-ACG |
| | GCGTCAG (SEQ ID NO: 140) | PAM silencing edit) |
| 141 | TCCTCTGCAAAACGGGAGCTCTGCTCGTCAAT | RTT*4, 8, 12 (AGG-to-AAG |
| | GCTTCAG (SEQ ID NO: 141) | PAM silencing edit) |
| 142 | GTCCTCTGCAAAACGGGAGCTCTGCTCGTCAA | RTT*1, 5, 9 (AGG-to-ATG PAM |
| | TGCATCAG (SEQ ID NO: 142) | silencing edit) |
| 143 | GTCCTCTGCAAAACGGGAGCTCTGCTCGTCAA | RTT*2, 6, 10, 13 |
| | TGCCTCAG (SEQ ID NO: 143) | |
| 144 | GTCCTCTGCAAAACGGGAGCTCTGCTCGTCAA | RTT*3, 7, 11 (AGG-to-ACG |
| | TGCGTCAG (SEQ ID NO: 144) | PAM silencing edit) |
| 145 | GTCCTCTGCAAAACGGGAGCTCTGCTCGTCAA | RTT*4, 8, 12 (AGG-to-AAG |
| | TGCTTCAG (SEQ ID NO: 145) | PAM silencing edit) |
| 146 | AACGGGAGCTCTGCTCGTCA (SEQ ID NO: 146) | PE3 ngRNA spacer (AGG |
| | | PAM) |
| 147 | AGCCACTGTCCTCTGCAAAA (SEQ ID NO: 147) | PE3 ngRNA spacer (CGG |
| | | PAM) |
| 148 | CAGCCTTGGGGACACCGTTG (SEQ ID NO: 148) | PE3 ngRNA spacer (TGG |
| | | PAM) |
| 149 | CCTCGGAGTTGTGACAGCCT (SEQ ID NO: 149) | PE3 ngRNA spacer (TGG |
| | | PAM) |
| 150 | CTCGGAGTIGTGACAGCCTT (SEQ ID NO: 150) | PE3 ngRNA spacer (GGG |
| | | PAM) |
| 151 | CTCTGCTCGTCAATGCATCA (SEQ ID NO: 151) | PE3b*1 ngRNA spacer (GGG |
| | | PAM) |
| 152 | CTCTGCTCGTCAATGCCTCA (SEQ ID NO: 152) | PE3b*2 ngRNA spacer (GGG |
| | | PAM) |
| 153 | CTCTGCTCGTCAATGCGTCA (SEQ ID NO: 153) | PE3b*3 ngRNA spacer (GGG |
| | | PAM) |
| 154 | CTCTGCTCGTCAATGCTTCA (SEQ ID NO: 154) | PE3b*4 ngRNA spacer (GGG |
| | | PAM) |
| 155 | GCCACTGTCCTCTGCAAAAC (SEQ ID NO: 155) | PE3 ngRNA spacer (GGG |
| | | PAM) |
| 156 | GCTCGTCAATGCATCAGGGC (SEQ ID NO: 156) | PE3b*5 ngRNA spacer (AGG |
| | | PAM) |
| 157 | GCTCGTCAATGCCTCAGGGC (SEQ ID NO: 157) | PE3b*6 ngRNA spacer (AGG |
| | | PAM) |
| 158 | GCTCGTCAATGCGTCAGGGC (SEQ ID NO: 158) | PE3b*7 ngRNA spacer (AGG |
| | | PAM) |
| 159 | GCTCGTCAATGCTTCAGGGC (SEQ ID NO: 159) | PE3b*8 ngRNA spacer (AGG |
| | | PAM) |
| 160 | GCTCTGCTCGTCAATGCATC (SEQ ID NO: 160) | PE3b*9 ngRNA spacer (AGG |
| | | PAM) |
| 161 | GCTCTGCTCGTCAATGCCTC (SEQ ID NO: 161) | PE3b*10 ngRNA spacer (AGG |
| | | PAM) |
| 162 | GCTCTGCTCGTCAATGCGTC (SEQ ID NO: 162) | PE3b*11 ngRNA spacer (AGG |
| | | PAM) |
| 163 | GCTCTGCTCGTCAATGCTTC (SEQ ID NO: 163) | PE3b*12 ngRNA spacer (AGG |
| | | PAM) |
| 164 | TCGGAGTTGTGACAGCCTTG (SEQ ID NO: 164) | PE3 ngRNA spacer (GGG |
| | | PAM) |
| 165 | TTCAGTTTTGCATGTGCCCT (SEQ ID NO: 165) | PE3 ngRNA spacer (CGG |
| | | PAM) |
| 166 | GTACGGGCTTTTCCACGGAG (SEQ ID NO: 166) | PE3 ngRNA spacer (AGG |
| | | PAM) |
| 167 | CCAAGGCTGTCACAACTCCG (SEQ ID NO: 167) | PE3 ngRNA spacer (AGG |
| | | PAM) |
| 168 | TACGGGCTTTTCCACGGAGA (SEQ ID NO: 168) | PE3 ngRNA spacer (GGG |
| | | PAM) |
| 169 | GCTCTGCTCGTCAAGGCCTC (SEQ ID NO: 169) | PE3 ngRNA spacer (AGG |
| | | PAM) |
| 170 | ATGCAGTACGGGCTTTTCCA (SEQ ID NO: 170) | PE3 ngRNA spacer (CGG |
| | | PAM) |
| 171 | CAGGAGCTGGACAAGTTTAT (SEQ ID NO: 171) | PE3 ngRNA spacer (GGG |
| | | PAM) |
| 172 | CTACTTACATGAGAACCGAA (SEQ ID NO: 172) | PE3 ngRNA spacer (AGG |
| | | PAM) |
| 173 | CAATGCCTCAGGGCAGGAGC (SEQ ID NO: 173) | PE3b*13 ngRNA spacer (TGG |
| | | PAM) |
| 174 | GGTTGGGAGCAAGGCCCTTT (SEQ ID NO: 174) | PE3 ngRNA spacer (CGG |
| | | PAM) |
| 175 | GAGAGGGTGTGAGGCAGTGT (SEQ ID NO: 175) | PE3 ngRNA spacer (GGG |
| | | PAM) |
| 176 | TTTATGGGTGAAATGCAGTA (SEQ ID NO: 176) | PE3 ngRNA spacer (CGG |
| | | PAM) |
| 177 | GGGTGTGAGGCAGTGTGGGT (SEQ ID NO: 177) | PE3 ngRNA spacer (TGG |
| | | PAM) |
| 178 | TACTTACATGAGAACCGAAA (SEQ ID NO: 178) | PE3 ngRNA spacer (GGG |
| | | PAM) |
| 179 | GGAGAGGGTGTGAGGCAGTG (SEQ ID NO: 179) | PE3 ngRNA spacer (TGG |
| | | PAM) |
| 180 | GCAGGAGCTGGACAAGTTTA (SEQ ID NO: 180) | PE3 ngRNA spacer (TGG |
| | | PAM) |
| 181 | CTCTGCTCGTCAATGGCTCA (SEQ ID NO: 181) | PE3b*14 ngRNA spacer (GGG |
| | | PAM) |
| 182 | TTATGGGTGAAATGCAGTAC (SEQ ID NO: 182) | PE3 ngRNA spacer (GGG |
| | | PAM) |
| 183 | GGCAGTGTGGGTTGGGAGCA (SEQ ID NO: 183) | PE3 ngRNA spacer (AGG |
| | | PAM) |
| 184 | GCTCGTCAAGGCCTCAGGGC (SEQ ID NO: 184) | PE3 ngRNA spacer (AGG |
| | | PAM) |
| 185 | ACTGCCTCACACCCTCTCCG (SEQ ID NO: 185) | PE3 ngRNA spacer (TGG |
| | | PAM) |
| 186 | GCTCGTCAATGGCTCAGGGC (SEQ ID NO: 186) | PE3b*15 ngRNA spacer (AGG |
| | | PAM) |
| 187 | TTTTCCACGGAGAGGGTGTG (SEQ ID NO: 187) | PE3 ngRNA spacer (AGG |
| | | PAM) |
| 188 | CAAGGCCTCAGGGCAGGAGC (SEQ ID NO: 188) | PE3 ngRNA spacer (TGG |
| | | PAM) |
| 189 | GATCCACAACGGTGTCCCCA (SEQ ID NO: 189) | PE3 ngRNA spacer (AGG |
| | | PAM) |
| 190 | ACAGTGGCTTTGATCCACAA (SEQ ID NO: 190) | PE3 ngRNA spacer (CGG |
| | | PAM) |
| 191 | ACATGCAAAACTGAACACTC (SEQ ID NO: 191) | PE3 ngRNA spacer (CGG |
| | | PAM) |
| 192 | CTCTGCTCGTCAAGGCCTCA (SEQ ID NO: 192) | PE3 ngRNA spacer (GGG |
| | | PAM) |
| 193 | CAAGGCTGTCACAACTCCGA (SEQ ID NO: 193) | PE3 ngRNA spacer (GGG |
| | | PAM) |
| 194 | GGTGTGAGGCAGTGTGGGTT (SEQ ID NO: 194) | PE3 ngRNA spacer (GGG |
| | | PAM) |
| 195 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CGTCAATGCCTCAGGGCAGGAG (SEQ ID NO: | |
| | 195) | |
| 196 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CGTCAATGCCTCAGGGCAGGAGC (SEQ ID NO: | |
| | 196) | |
| 197 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CTCAATGCCTCAGGGCAGGAGCT (SEQ ID NO: | |
| | 197) | |
| 198 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit) |
| | CTCAATGGCTCAGGGCAGGAGCT (SEQ ID NO: | |
| | 198) | |
| 199 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit) |
| | CGTCAATGGCTCAGGGCAGGAGC (SEQ ID NO: | |
| | 199) | |
| 200 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit) |
| | ATCAACTTGAAAAAGTGGCACCGAGTCGGTG | |
| | CTCAATGCGTCAGGGCAGGAGCT (SEQ ID NO: | |
| | 200) | |
| 201 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit) |
| | ATCAACTTGAAAAAGTGGCACCGAGTCGGTG | |
| | CGTCAATGCGTCAGGGCAGGAGC (SEQ ID NO: | |
| | 201) | |
| 202 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CTCGTCAATGCCTCAGGGCAGGAG (SEQ ID | |
| | NO: 202) | |
| 203 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CGTCAATGCCTCAGGGCAGGAGCT (SEQ ID | |
| | NO: 203) | |
| 204 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CTCAATGCCTCAGGGCAGGAGCTG (SEQ ID | |
| | NO: 204) | |
| 205 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit) |
| | CTCAATGGCTCAGGGCAGGAGCTG (SEQ ID | |
| | NO: 205) | |
| 206 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit) |
| | CGTCAATGGCTCAGGGCAGGAGCT (SEQ ID | |
| | NO: 206) | |
| 207 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit) |
| | ATCAACTTGAAAAAGTGGCACCGAGTCGGTG | |
| | CTCAATGCGTCAGGGCAGGAGCTG (SEQ ID | |
| | NO: 207) | |
| 208 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit) |
| | ATCAACTTGAAAAAGTGGCACCGAGTCGGTG | |
| | CGTCAATGCGTCAGGGCAGGAGCT (SEQ ID | |
| | NO: 208) | |
| 209 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CGTCAATGCCTCAGGGCAGGAGCTG (SEQ ID | |
| | NO: 209) | |
| 210 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CTCGTCAATGCCTCAGGGCAGGAGC (SEQ ID | |
| | NO: 210) | |
| 211 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CTCAATGCCTCAGGGCAGGAGCTGG (SEQ ID | |
| | NO: 211) | |
| 212 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit) |
| | CTCAATGGCTCAGGGCAGGAGCTGG (SEQ ID | |
| | NO: 212) | |
| 213 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit) |
| | CGTCAATGGCTCAGGGCAGGAGCTG (SEQ ID | |
| | NO: 213) | |
| 214 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit) |
| | CTCGTCAATGGCTCAGGGCAGGAGC (SEQ ID | |
| | NO: 214) | |
| 215 | CTTGTCCAGCTCCTGCCCTGGTTTTAGAGCTA | pegRNA*13 (G-to-T |
| | GAAATAGCAAGTTAAAATAAGGCTAGTCCGTT | nonsynonymous [V47F] edit) |
| | ATCAACTTGAAAAAGTGGCACCGAGTCGGTG | |
| | CTTCAATGCCTCAGGGCAGGAGCTG (SEQ ID | |
| | NO: 215) | |
| 216 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit) |
| | ATCAACTTGAAAAAGTGGCACCGAGTCGGTG | |
| | CTCAATGCGTCAGGGCAGGAGCTGG (SEQ ID | |
| | NO: 216) | |
| 217 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit) |
| | ATCAACTTGAAAAAGTGGCACCGAGTCGGTG | |
| | CGTCAATGCGTCAGGGCAGGAGCTG (SEQ ID | |
| | NO: 217) | |
| 218 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit) |
| | ATCAACTTGAAAAAGTGGCACCGAGTCGGTG | |
| | CTCGTCAATGCGTCAGGGCAGGAGC (SEQ ID | |
| | NO: 218) | |
| 219 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CTCGTCAATGCCTCAGGGCAGGAGCT (SEQ ID | |
| | NO: 219) | |
| 220 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CGTCAATGCCTCAGGGCAGGAGCTGG (SEQ ID | |
| | NO: 220) | |
| 221 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CGCTCGTCAATGCCTCAGGGCAGGAG (SEQ ID | |
| | NO: 221) | |
| 222 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CTCAATGCCTCAGGGCAGGAGCTGGA (SEQ ID | |
| | NO: 222) | |
| 223 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit) |
| | CTCAATGGCTCAGGGCAGGAGCTGGA (SEQ ID | |
| | NO: 223) | |
| 224 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit) |
| | CGTCAATGGCTCAGGGCAGGAGCTGG (SEQ ID | |
| | NO: 224) | |
| 225 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit) |
| | CTCGTCAATGGCTCAGGGCAGGAGCT (SEQ ID | |
| | NO: 225) | |
| 226 | CTTGTCCAGCTCCTGCCCTGGTTTTAGAGCTA | pegRNA (G-to-T |
| | GAAATAGCAAGTTAAAATAAGGCTAGTCCGTT | nonsynonymous [V47F]; AGG- |
| | ATCAACTTGAAAAAGTGGCACCGAGTCGGTG | to-AGC nonsynonymous |
| | CTTCAATGGCTCAGGGCAGGAGCTGG (SEQ ID | [A49G] PAM silencing edit) |
| | NO: 226) | |
| 227 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit) |
| | ATCAACTTGAAAAAGTGGCACCGAGTCGGTG | |
| | CTCAATGCGTCAGGGCAGGAGCTGGA (SEQ ID | |
| | NO: 227) | |
| 228 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit) |
| | ATCAACTTGAAAAAGTGGCACCGAGTCGGTG | |
| | CGTCAATGCGTCAGGGCAGGAGCTGG (SEQ ID | |
| | NO: 228) | |
| 229 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit) |
| | ATCAACTTGAAAAAGTGGCACCGAGTCGGTG | |
| | CTCGTCAATGCGTCAGGGCAGGAGCT (SEQ ID | |
| | NO: 229) | |
| 230 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CTGCTCGTCAATGCCTCAGGGCAGGAG (SEQ | |
| | ID NO: 230) | |
| 231 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CGTCAATGCCTCAGGGCAGGAGCTGGA (SEQ | |
| | ID NO: 231) | |
| 232 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CTCGTCAATGCCTCAGGGCAGGAGCTG (SEQ | |
| | ID NO: 232) | |
| 233 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CGCTCGTCAATGCCTCAGGGCAGGAGC (SEQ | |
| | ID NO: 233) | |
| 234 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CTCAATGCCTCAGGGCAGGAGCTGGAC (SEQ | |
| | ID NO: 234) | |
| 235 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit) |
| | CTCAATGGCTCAGGGCAGGAGCTGGAC (SEQ | |
| | ID NO: 235) | |
| 236 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit) |
| | CGTCAATGGCTCAGGGCAGGAGCTGGA (SEQ | |
| | ID NO: 236) | |
| 237 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit) |
| | CTCGTCAATGGCTCAGGGCAGGAGCTG (SEQ | |
| | ID NO: 237) | |
| 238 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit) |
| | CGCTCGTCAATGGCTCAGGGCAGGAGC (SEQ | |
| | ID NO: 238) | |
| 239 | CTTGTCCAGCTCCTGCCCTGGTTTTAGAGCTA | pegRNA (G-to-T |
| | GAAATAGCAAGTTAAAATAAGGCTAGTCCGTT | nonsynonymous [V47F]; AGG- |
| | ATCAACTTGAAAAAGTGGCACCGAGTCGGTG | to-AGC nonsynonymous |
| | CTTCAATGGCTCAGGGCAGGAGCTGGA (SEQ | [A49G] PAM silencing edit) |
| | ID NO: 239) | |
| 240 | CTTGTCCAGCTCCTGCCCTGGTTTTAGAGCTA | pegRNA*13 (G-to-T |
| | GAAATAGCAAGTTAAAATAAGGCTAGTCCGTT | nonsynonymous [V47F] edit) |
| | ATCAACTTGAAAAAGTGGCACCGAGTCGGTG | |
| | CTTCAATGCCTCAGGGCAGGAGCTGGA (SEQ | |
| | ID NO: 240) | |
| 241 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit) |
| | ATCAACTTGAAAAAGTGGCACCGAGTCGGTG | |
| | CTCAATGCGTCAGGGCAGGAGCTGGAC (SEQ | |
| | ID NO: 241) | |
| 242 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit) |
| | ATCAACTTGAAAAAGTGGCACCGAGTCGGTG | |
| | CGTCAATGCGTCAGGGCAGGAGCTGGA (SEQ | |
| | ID NO: 242) | |
| 243 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit) |
| | ATCAACTTGAAAAAGTGGCACCGAGTCGGTG | |
| | CTCGTCAATGCGTCAGGGCAGGAGCTG (SEQ | |
| | ID NO: 243) | |
| 244 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit) |
| | ATCAACTTGAAAAAGTGGCACCGAGTCGGTG | |
| | CGCTCGTCAATGCGTCAGGGCAGGAGC (SEQ | |
| | ID NO: 244) | |
| 245 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CTCGTCAATGCCTCAGGGCAGGAGCTGG (SEQ | |
| | ID NO: 245) | |
| 246 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CGTCAATGCCTCAGGGCAGGAGCTGGAC (SEQ | |
| | ID NO: 246) | |
| 247 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CGCTCGTCAATGCCTCAGGGCAGGAGCT (SEQ | |
| | ID NO: 247) | |
| 248 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CTGCTCGTCAATGCCTCAGGGCAGGAGC (SEQ | |
| | ID NO: 248) | |
| 249 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit) |
| | ATCAACTTGAAAAAGTGGCACCGAGTCGGTG | |
| | CTCGTCAATGCGTCAGGGCAGGAGCTGG (SEQ | |
| | ID NO: 249) | |
| 250 | CTTGTCCAGCTCCTGCCCTGGTTTTAGAGCTA | pegRNA*1, 5, 9 (AGG-to-ATG |
| | GAAATAGCAAGTTAAAATAAGGCTAGTCCGTT | PAM silencing edit) |
| | ATCAACTTGAAAAAGTGGCACCGAGTCGGTG | |
| | CTCGTCAATGCATCAGGGCAGGAGCTGG (SEQ | |
| | ID NO: 250) | |
| 251 | CTTGTCCAGCTCCTGCCCTGGTTTTAGAGCTA | pegRNA*4, 8, 12 (AGG-to-AAG |
| | GAAATAGCAAGTTAAAATAAGGCTAGTCCGTT | PAM silencing edit) |
| | ATCAACTTGAAAAAGTGGCACCGAGTCGGTG | |
| | CTCGTCAATGCTTCAGGGCAGGAGCTGG (SEQ | |
| | ID NO: 251) | |
| 252 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CTCAATGCCTCAGGGCAGGAGCTGGACA (SEQ | |
| | ID NO: 252) | |
| 253 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit) |
| | CTCAATGGCTCAGGGCAGGAGCTGGACA (SEQ | |
| | ID NO: 253) | |
| 254 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit) |
| | CGTCAATGGCTCAGGGCAGGAGCTGGAC (SEQ | |
| | ID NO: 254) | |
| 255 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit) |
| | CTCGTCAATGGCTCAGGGCAGGAGCTGG (SEQ | |
| | ID NO: 255) | |
| 256 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit) |
| | CGCTCGTCAATGGCTCAGGGCAGGAGCT (SEQ | |
| | ID NO: 256) | |
| 257 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit) |
| | CTGCTCGTCAATGGCTCAGGGCAGGAGC (SEQ | |
| | ID NO: 257) | |
| 258 | CTTGTCCAGCTCCTGCCCTGGTTTTAGAGCTA | pegRNA (G-to-T synonymous; |
| | GAAATAGCAAGTTAAAATAAGGCTAGTCCGTT | AGG-to-AGC nonsynonymous |
| | ATCAACTTGAAAAAGTGGCACCGAGTCGGTG | [A49G] PAM silencing edit) |
| | CTCTCGTCAATGGCTCAGGGCAGGAGCT (SEQ | |
| | ID NO: 258) | |
| 259 | CTTGTCCAGCTCCTGCCCTGGTTTTAGAGCTA | pegRNA (G-to-T |
| | GAAATAGCAAGTTAAAATAAGGCTAGTCCGTT | nonsynonymous [V47F]; AGG- |
| | ATCAACTTGAAAAAGTGGCACCGAGTCGGTG | to-AGC nonsynonymous |
| | CTTCAATGGCTCAGGGCAGGAGCTGGAC (SEQ | [A49G] PAM silencing edit) |
| | ID NO: 259) | |
| 260 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit) |
| | ATCAACTTGAAAAAGTGGCACCGAGTCGGTG | |
| | CTCAATGCGTCAGGGCAGGAGCTGGACA (SEQ | |
| | ID NO: 260) | |
| 261 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit) |
| | ATCAACTTGAAAAAGTGGCACCGAGTCGGTG | |
| | CGTCAATGCGTCAGGGCAGGAGCTGGAC (SEQ | |
| | ID NO: 261) | |
| 262 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit) |
| | ATCAACTTGAAAAAGTGGCACCGAGTCGGTG | |
| | CGCTCGTCAATGCGTCAGGGCAGGAGCT (SEQ | |
| | ID NO: 262) | |
| 263 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit) |
| | ATCAACTTGAAAAAGTGGCACCGAGTCGGTG | |
| | CTGCTCGTCAATGCGTCAGGGCAGGAGC (SEQ | |
| | ID NO: 263) | |
| 264 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CTGCTCGTCAATGCCTCAGGGCAGGAGCT | |
| | (SEQ ID NO: 264) | |
| 265 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CGTCAATGCCTCAGGGCAGGAGCTGGACA | |
| | (SEQ ID NO: 265) | |
| 266 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CTCGTCAATGCCTCAGGGCAGGAGCTGGA | |
| | (SEQ ID NO: 266) | |
| 267 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CGCTCGTCAATGCCTCAGGGCAGGAGCTG | |
| | (SEQ ID NO: 267) | |
| 268 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CTCTGCTCGTCAATGCCTCAGGGCAGGAG | |
| | (SEQ ID NO: 268) | |
| 269 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit) |
| | CGTCAATGGCTCAGGGCAGGAGCTGGACA | |
| | (SEQ ID NO: 269) | |
| 270 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit) |
| | CTCGTCAATGGCTCAGGGCAGGAGCTGGA | |
| | (SEQ ID NO: 270) | |
| 271 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit) |
| | CGCTCGTCAATGGCTCAGGGCAGGAGCTG | |
| | (SEQ ID NO: 271) | |
| 272 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit) |
| | CTGCTCGTCAATGGCTCAGGGCAGGAGCT | |
| | (SEQ ID NO: 272) | |
| 273 | CTTGTCCAGCTCCTGCCCTGGTTTTAGAGCTA | pegRNA (G-to-T synonymous; |
| | GAAATAGCAAGTTAAAATAAGGCTAGTCCGTT | AGG-to-AGC nonsynonymous |
| | ATCAACTTGAAAAAGTGGCACCGAGTCGGTG | [A49G] PAM silencing edit) |
| | CTCTCGTCAATGGCTCAGGGCAGGAGCTG | |
| | (SEQ ID NO: 273) | |
| 274 | CTTGTCCAGCTCCTGCCCTGGTTTTAGAGCTA | pegRNA (G-to-T |
| | GAAATAGCAAGTTAAAATAAGGCTAGTCCGTT | nonsynonymous [V47F]; AGG- |
| | ATCAACTTGAAAAAGTGGCACCGAGTCGGTG | to-AGC nonsynonymous |
| | CTTCAATGGCTCAGGGCAGGAGCTGGACA | [A49G] PAM silencing edit) |
| | (SEQ ID NO: 274) | |
| 275 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit) |
| | ATCAACTTGAAAAAGTGGCACCGAGTCGGTG | |
| | CGTCAATGCGTCAGGGCAGGAGCTGGACA | |
| | (SEQ ID NO: 275) | |
| 276 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit) |
| | ATCAACTTGAAAAAGTGGCACCGAGTCGGTG | |
| | CTCGTCAATGCGTCAGGGCAGGAGCTGGA | |
| | (SEQ ID NO: 276) | |
| 277 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit) |
| | ATCAACTTGAAAAAGTGGCACCGAGTCGGTG | |
| | CGCTCGTCAATGCGTCAGGGCAGGAGCTG | |
| | (SEQ ID NO: 277) | |
| 278 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit) |
| | ATCAACTTGAAAAAGTGGCACCGAGTCGGTG | |
| | CTGCTCGTCAATGCGTCAGGGCAGGAGCT | |
| | (SEQ ID NO: 278) | |
| 279 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CTCGTCAATGCCTCAGGGCAGGAGCTGGAC | |
| | (SEQ ID NO: 279) | |
| 280 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CGCTCGTCAATGCCTCAGGGCAGGAGCTGG | |
| | (SEQ ID NO: 280) | |
| 281 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CTGCTCGTCAATGCCTCAGGGCAGGAGCTG | |
| | (SEQ ID NO: 281) | |
| 282 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CTCTGCTCGTCAATGCCTCAGGGCAGGAGC | |
| | (SEQ ID NO: 282) | |
| 283 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CGTCAATGCCTCAGGGCAGGAGCTGGACAA | |
| | (SEQ ID NO: 283) | |
| 284 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit) |
| | CTCGTCAATGGCTCAGGGCAGGAGCTGGAC | |
| | (SEQ ID NO: 284) | |
| 285 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit) |
| | CGCTCGTCAATGGCTCAGGGCAGGAGCTGG | |
| | (SEQ ID NO: 285) | |
| 286 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit) |
| | CTGCTCGTCAATGGCTCAGGGCAGGAGCTG | |
| | (SEQ ID NO: 286) | |
| 287 | CTTGTCCAGCTCCTGCCCTGGTTTTAGAGCTA | pegRNA (G-to-T synonymous; |
| | GAAATAGCAAGTTAAAATAAGGCTAGTCCGTT | AGG-to-AGC nonsynonymous |
| | ATCAACTTGAAAAAGTGGCACCGAGTCGGTG | [A49G] PAM silencing edit) |
| | CTCTCGTCAATGGCTCAGGGCAGGAGCTGG | |
| | (SEQ ID NO: 287) | |
| 288 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit) |
| | ATCAACTTGAAAAAGTGGCACCGAGTCGGTG | |
| | CTCGTCAATGCGTCAGGGCAGGAGCTGGAC | |
| | (SEQ ID NO: 288) | |
| 289 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit) |
| | ATCAACTTGAAAAAGTGGCACCGAGTCGGTG | |
| | CGCTCGTCAATGCGTCAGGGCAGGAGCTGG | |
| | (SEQ ID NO: 289) | |
| 290 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit) |
| | ATCAACTTGAAAAAGTGGCACCGAGTCGGTG | |
| | CTGCTCGTCAATGCGTCAGGGCAGGAGCTG | |
| | (SEQ ID NO: 290) | |
| 291 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CGCTCTGCTCGTCAATGCCTCAGGGCAGGAG | |
| | (SEQ ID NO: 291) | |
| 292 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CTGCTCGTCAATGCCTCAGGGCAGGAGCTGG | |
| | (SEQ ID NO: 292) | |
| 293 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CTCGTCAATGCCTCAGGGCAGGAGCTGGACA | |
| | (SEQ ID NO: 293) | |
| 294 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CGCTCGTCAATGCCTCAGGGCAGGAGCTGGA | |
| | (SEQ ID NO: 294) | |
| 295 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CTCTGCTCGTCAATGCCTCAGGGCAGGAGCT | |
| | (SEQ ID NO: 295) | |
| 296 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit) |
| | CTCGTCAATGGCTCAGGGCAGGAGCTGGACA | |
| | (SEQ ID NO: 296) | |
| 297 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit) |
| | CGCTCGTCAATGGCTCAGGGCAGGAGCTGGA | |
| | (SEQ ID NO: 297) | |
| 298 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit) |
| | CTGCTCGTCAATGGCTCAGGGCAGGAGCTGG | |
| | (SEQ ID NO: 298) | |
| 299 | CTTGTCCAGCTCCTGCCCTGGTTTTAGAGCTA | pegRNA (G-to-T synonymous; |
| | GAAATAGCAAGTTAAAATAAGGCTAGTCCGTT | AGG-to-AGC nonsynonymous |
| | ATCAACTTGAAAAAGTGGCACCGAGTCGGTG | [A49G] PAM silencing edit) |
| | CTCTCGTCAATGGCTCAGGGCAGGAGCTGGA | |
| | (SEQ ID NO: 299) | |
| 300 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit) |
| | ATCAACTTGAAAAAGTGGCACCGAGTCGGTG | |
| | CTCGTCAATGCGTCAGGGCAGGAGCTGGACA | |
| | (SEQ ID NO: 300) | |
| 301 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit) |
| | ATCAACTTGAAAAAGTGGCACCGAGTCGGTG | |
| | CGCTCGTCAATGCGTCAGGGCAGGAGCTGGA | |
| | (SEQ ID NO: 301) | |
| 302 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit) |
| | ATCAACTTGAAAAAGTGGCACCGAGTCGGTG | |
| | CTGCTCGTCAATGCGTCAGGGCAGGAGCTGG | |
| | (SEQ ID NO: 302) | |
| 303 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CGCTCGTCAATGCCTCAGGGCAGGAGCTGGAC | |
| | (SEQ ID NO: 303) | |
| 304 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CTGCTCGTCAATGCCTCAGGGCAGGAGCTGGA | |
| | (SEQ ID NO: 304) | |
| 305 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CTCTGCTCGTCAATGCCTCAGGGCAGGAGCTG | |
| | (SEQ ID NO: 305) | |
| 306 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CGCTCTGCTCGTCAATGCCTCAGGGCAGGAGC | |
| | (SEQ ID NO: 306) | |
| 307 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CTCGTCAATGCCTCAGGGCAGGAGCTGGACA | |
| | A (SEQ ID NO: 307) | |
| 308 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit) |
| | CGCTCGTCAATGGCTCAGGGCAGGAGCTGGA | |
| | C (SEQ ID NO: 308) | |
| 309 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit) |
| | CTGCTCGTCAATGGCTCAGGGCAGGAGCTGGA | |
| | (SEQ ID NO: 309) | |
| 310 | CTTGTCCAGCTCCTGCCCTGGTTTTAGAGCTA | pegRNA (G-to-T synonymous; |
| | GAAATAGCAAGTTAAAATAAGGCTAGTCCGTT | AGG-to-AGC nonsynonymous |
| | ATCAACTTGAAAAAGTGGCACCGAGTCGGTG | [A49G] PAM silencing edit) |
| | CTCTCGTCAATGGCTCAGGGCAGGAGCTGGAC | |
| | (SEQ ID NO: 310) | |
| 311 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit) |
| | ATCAACTTGAAAAAGTGGCACCGAGTCGGTG | |
| | CGCTCGTCAATGCGTCAGGGCAGGAGCTGGA | |
| | C (SEQ ID NO: 311) | |
| 312 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit) |
| | ATCAACTTGAAAAAGTGGCACCGAGTCGGTG | |
| | CTGCTCGTCAATGCGTCAGGGCAGGAGCTGGA | |
| | (SEQ ID NO: 312) | |
| 313 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CGAGCTCTGCTCGTCAATGCCTCAGGGCAGGA | |
| | G (SEQ ID NO: 313) | |
| 314 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CGCTCTGCTCGTCAATGCCTCAGGGCAGGAGC | |
| | T (SEQ ID NO: 314) | |
| 315 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CGCTCGTCAATGCCTCAGGGCAGGAGCTGGAC | |
| | A (SEQ ID NO: 315) | |
| 316 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CTGCTCGTCAATGCCTCAGGGCAGGAGCTGGA | |
| | C (SEQ ID NO: 316) | |
| 317 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CTCTGCTCGTCAATGCCTCAGGGCAGGAGCTG | |
| | G (SEQ ID NO: 317) | |
| 318 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit) |
| | CGCTCGTCAATGGCTCAGGGCAGGAGCTGGA | |
| | CA (SEQ ID NO: 318) | |
| 319 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit) |
| | CTGCTCGTCAATGGCTCAGGGCAGGAGCTGGA | |
| | C (SEQ ID NO: 319) | |
| 320 | CTTGTCCAGCTCCTGCCCTGGTTTTAGAGCTA | pegRNA (G-to-T synonymous; |
| | GAAATAGCAAGTTAAAATAAGGCTAGTCCGTT | AGG-to-AGC nonsynonymous |
| | ATCAACTTGAAAAAGTGGCACCGAGTCGGTG | [A49G] PAM silencing edit) |
| | CTCTCGTCAATGGCTCAGGGCAGGAGCTGGAC | |
| | A (SEQ ID NO: 320) | |
| 321 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit) |
| | ATCAACTTGAAAAAGTGGCACCGAGTCGGTG | |
| | CGCTCGTCAATGCGTCAGGGCAGGAGCTGGA | |
| | CA (SEQ ID NO: 321) | |
| 322 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit) |
| | ATCAACTTGAAAAAGTGGCACCGAGTCGGTG | |
| | CTGCTCGTCAATGCGTCAGGGCAGGAGCTGGA | |
| | C (SEQ ID NO: 322) | |
| 323 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CGCTCGTCAATGCCTCAGGGCAGGAGCTGGAC | |
| | AA (SEQ ID NO: 323) | |
| 324 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CTGCTCGTCAATGCCTCAGGGCAGGAGCTGGA | |
| | CA (SEQ ID NO: 324) | |
| 325 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CTCTGCTCGTCAATGCCTCAGGGCAGGAGCTG | |
| | GA (SEQ ID NO: 325) | |
| 326 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CGCTCTGCTCGTCAATGCCTCAGGGCAGGAGC | |
| | TG (SEQ ID NO: 326) | |
| 327 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CGAGCTCTGCTCGTCAATGCCTCAGGGCAGGA | |
| | GC (SEQ ID NO: 327) | |
| 328 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit) |
| | CTGCTCGTCAATGGCTCAGGGCAGGAGCTGGA | |
| | CA (SEQ ID NO: 328) | |
| 329 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit) |
| | ATCAACTTGAAAAAGTGGCACCGAGTCGGTG | |
| | CTGCTCGTCAATGCGTCAGGGCAGGAGCTGGA | |
| | CA (SEQ ID NO: 329) | |
| 330 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CGGGAGCTCTGCTCGTCAATGCCTCAGGGCAG | |
| | GAG (SEQ ID NO: 330) | |
| 331 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CGAGCTCTGCTCGTCAATGCCTCAGGGCAGGA | |
| | GCT (SEQ ID NO: 331) | |
| 332 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CGCTCTGCTCGTCAATGCCTCAGGGCAGGAGC | |
| | TGG (SEQ ID NO: 332) | |
| 333 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CTCTGCTCGTCAATGCCTCAGGGCAGGAGCTG | |
| | GAC (SEQ ID NO: 333) | |
| 334 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CTGCTCGTCAATGCCTCAGGGCAGGAGCTGGA | |
| | CAA (SEQ ID NO: 334) | |
| 335 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CTCTGCTCGTCAATGCCTCAGGGCAGGAGCTG | |
| | GACA (SEQ ID NO: 335) | |
| 336 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CGCTCTGCTCGTCAATGCCTCAGGGCAGGAGC | |
| | TGGA (SEQ ID NO: 336) | |
| 337 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CGAGCTCTGCTCGTCAATGCCTCAGGGCAGGA | |
| | GCTG (SEQ ID NO: 337) | |
| 338 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CGGGAGCTCTGCTCGTCAATGCCTCAGGGCAG | |
| | GAGC (SEQ ID NO: 338) | |
| 339 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CGGGAGCTCTGCTCGTCAATGCCTCAGGGCAG | |
| | GAGCT (SEQ ID NO: 339) | |
| 340 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CGAGCTCTGCTCGTCAATGCCTCAGGGCAGGA | |
| | GCTGG (SEQ ID NO: 340) | |
| 341 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CGCTCTGCTCGTCAATGCCTCAGGGCAGGAGC | |
| | TGGAC (SEQ ID NO: 341) | |
| 342 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CTCTGCTCGTCAATGCCTCAGGGCAGGAGCTG | |
| | GACAA (SEQ ID NO: 342) | |
| 343 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CGCTCTGCTCGTCAATGCCTCAGGGCAGGAGC | |
| | TGGACA (SEQ ID NO: 343) | |
| 344 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CGAGCTCTGCTCGTCAATGCCTCAGGGCAGGA | |
| | GCTGGA (SEQ ID NO: 344) | |
| 345 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CGGGAGCTCTGCTCGTCAATGCCTCAGGGCAG | |
| | GAGCTGG (SEQ ID NO: 345) | |
| 346 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CGAGCTCTGCTCGTCAATGCCTCAGGGCAGGA | |
| | GCTGGAC (SEQ ID NO: 346) | |
| 347 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CGCTCTGCTCGTCAATGCCTCAGGGCAGGAGC | |
| | TGGACAA (SEQ ID NO: 347) | |
| 348 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CGAGCTCTGCTCGTCAATGCCTCAGGGCAGGA | |
| | GCTGGACA (SEQ ID NO: 348) | |
| 349 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CGGGAGCTCTGCTCGTCAATGCCTCAGGGCAG | |
| | GAGCTGGAC (SEQ ID NO: 349) | |
| 350 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CGAGCTCTGCTCGTCAATGCCTCAGGGCAGGA | |
| | GCTGGACAA (SEQ ID NO: 350) | |
| 351 | CTTGTCCAGCTCCTGCCCTGGTTTTAGAGCTA | pegRNA*2, 6, 10, 13 |
| | GAAATAGCAAGTTAAAATAAGGCTAGTCCGTT | |
| | ATCAACTTGAAAAAGTGGCACCGAGTCGGTG | |
| | CGGGAGCTCTGCTCGTCAATGCCTCAGGGCAG | |
| | GAGCTGGACAA (SEQ ID NO: 351) | |
| 352 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CGTCAATGCCTCAGGGCAGGAGAACATTGAC | |
| | GCGTCTCTACGTGGGGGCGCG (SEQ ID NO: | |
| | 352) | |
| 353 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CGTCAATGCCTCAGGGCAGGAGCAACATTGA | |
| | CGCGTCTCTACGTGGGGGCGCG (SEQ ID NO: | |
| | 353) | |
| 354 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CTCAATGCCTCAGGGCAGGAGCTAACATTGAC | |
| | GCGTCTCTACGTGGGGGCGCG (SEQ ID NO: | |
| | 354) | |
| 355 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit); includes linker + |
| | CTCAATGGCTCAGGGCAGGAGCTAACATTGAC | hairpin |
| | GCGTCTCTACGTGGGGGCGCG (SEQ ID NO: | |
| | 355) | |
| 356 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit); includes linker + |
| | CGTCAATGGCTCAGGGCAGGAGCAACATTGA | hairpin |
| | CGCGTCTCTACGTGGGGGCGCG (SEQ ID NO: | |
| | 356) | |
| 357 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit); includes |
| | ATCAACTTGAAAAAGTGGCACCGAGTCGGTG | linker + hairpin |
| | CTCAATGCGTCAGGGCAGGAGCTAACATTGAC | |
| | GCGTCTCTACGTGGGGGCGCG (SEQ ID NO: | |
| | 357) | |
| 358 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit); includes |
| | ATCAACTTGAAAAAGTGGCACCGAGTCGGTG | linker + hairpin |
| | CGTCAATGCGTCAGGGCAGGAGCAACATTGA | |
| | CGCGTCTCTACGTGGGGGCGCG (SEQ ID NO: | |
| | 358) | |
| 359 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CTCGTCAATGCCTCAGGGCAGGAGAACATTGA | |
| | CGCGTCTCTACGTGGGGGCGCG (SEQ ID NO: | |
| | 359) | |
| 360 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CGTCAATGCCTCAGGGCAGGAGCTAACATTGA | |
| | CGCGTCTCTACGTGGGGGCGCG (SEQ ID NO: | |
| | 360) | |
| 361 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CTCAATGCCTCAGGGCAGGAGCTGAACATTGA | |
| | CGCGTCTCTACGTGGGGGCGCG (SEQ ID NO: | |
| | 361) | |
| 362 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit); includes linker + |
| | CTCAATGGCTCAGGGCAGGAGCTGAACATTG | hairpin |
| | ACGCGTCTCTACGTGGGGGCGCG (SEQ ID NO: | |
| | 362) | |
| 363 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit); includes linker + |
| | CGTCAATGGCTCAGGGCAGGAGCTAACATTG | hairpin |
| | ACGCGTCTCTACGTGGGGGCGCG (SEQ ID NO: | |
| | 363) | |
| 364 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit); includes |
| | ATCAACTTGAAAAAGTGGCACCGAGTCGGTG | linker + hairpin |
| | CTCAATGCGTCAGGGCAGGAGCTGAACATTG | |
| | ACGCGTCTCTACGTGGGGGCGCG (SEQ ID NO: | |
| | 364) | |
| 365 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit); includes |
| | ATCAACTTGAAAAAGTGGCACCGAGTCGGTG | linker + hairpin |
| | CGTCAATGCGTCAGGGCAGGAGCTAACATTG | |
| | ACGCGTCTCTACGTGGGGGCGCG (SEQ ID NO: | |
| | 365) | |
| 366 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CGTCAATGCCTCAGGGCAGGAGCTGAACATTG | |
| | ACGCGTCTCTACGTGGGGGCGCG (SEQ ID NO: | |
| | 366) | |
| 367 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CTCGTCAATGCCTCAGGGCAGGAGCAACATTG | |
| | ACGCGTCTCTACGTGGGGGCGCG (SEQ ID NO: | |
| | 367) | |
| 368 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CTCAATGCCTCAGGGCAGGAGCTGGAACATTG | |
| | ACGCGTCTCTACGTGGGGGCGCG (SEQ ID NO: | |
| | 368) | |
| 369 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit); includes linker + |
| | CTCAATGGCTCAGGGCAGGAGCTGGAACATT | hairpin |
| | GACGCGTCTCTACGTGGGGGCGCG (SEQ ID | |
| | NO: 369) | |
| 370 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit); includes linker + |
| | CGTCAATGGCTCAGGGCAGGAGCTGAACATT | hairpin |
| | GACGCGTCTCTACGTGGGGGCGCG (SEQ ID | |
| | NO: 370) | |
| 371 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit); includes linker + |
| | CTCGTCAATGGCTCAGGGCAGGAGCAACATTG | hairpin |
| | ACGCGTCTCTACGTGGGGGCGCG (SEQ ID NO: | |
| | 371) | |
| 372 | CTTGTCCAGCTCCTGCCCTGGTTTTAGAGCTA | pegRNA*13 (G-to-T |
| | GAAATAGCAAGTTAAAATAAGGCTAGTCCGTT | nonsynonymous [V47F] edit); |
| | ATCAACTTGAAAAAGTGGCACCGAGTCGGTG | includes linker + hairpin |
| | CTTCAATGCCTCAGGGCAGGAGCTGAACATTG | |
| | ACGCGTCTCTACGTGGGGGCGCG (SEQ ID NO: | |
| | 372) | |
| 373 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit); includes |
| | ATCAACTTGAAAAAGTGGCACCGAGTCGGTG | linker + hairpin |
| | CTCAATGCGTCAGGGCAGGAGCTGGAACATT | |
| | GACGCGTCTCTACGTGGGGGCGCG (SEQ ID | |
| | NO: 373) | |
| 374 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit); includes |
| | ATCAACTTGAAAAAGTGGCACCGAGTCGGTG | linker + hairpin |
| | CGTCAATGCGTCAGGGCAGGAGCTGAACATT | |
| | GACGCGTCTCTACGTGGGGGCGCG (SEQ ID | |
| | NO: 374) | |
| 375 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit); includes |
| | ATCAACTTGAAAAAGTGGCACCGAGTCGGTG | linker + hairpin |
| | CTCGTCAATGCGTCAGGGCAGGAGCAACATTG | |
| | ACGCGTCTCTACGTGGGGGCGCG (SEQ ID NO: | |
| | 375) | |
| 376 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CTCGTCAATGCCTCAGGGCAGGAGCTAACATT | |
| | GACGCGTCTCTACGTGGGGGCGCG (SEQ ID | |
| | NO: 376) | |
| 377 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CGTCAATGCCTCAGGGCAGGAGCTGGAACATT | |
| | GACGCGTCTCTACGTGGGGGCGCG (SEQ ID | |
| | NO: 377) | |
| 378 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CGCTCGTCAATGCCTCAGGGCAGGAGAACATT | |
| | GACGCGTCTCTACGTGGGGGCGCG (SEQ ID | |
| | NO: 378) | |
| 379 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CTCAATGCCTCAGGGCAGGAGCTGGAAACATT | |
| | GACGCGTCTCTACGTGGGGGCGCG (SEQ ID | |
| | NO: 379) | |
| 380 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit); includes linker + |
| | CTCAATGGCTCAGGGCAGGAGCTGGAAACAT | hairpin |
| | TGACGCGTCTCTACGTGGGGGCGCG (SEQ ID | |
| | NO: 380) | |
| 381 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit); includes linker + |
| | CGTCAATGGCTCAGGGCAGGAGCTGGAACAT | hairpin |
| | TGACGCGTCTCTACGTGGGGGCGCG (SEQ ID | |
| | NO: 381) | |
| 382 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit); includes linker + |
| | CTCGTCAATGGCTCAGGGCAGGAGCTAACATT | hairpin |
| | GACGCGTCTCTACGTGGGGGCGCG (SEQ ID | |
| | NO: 382) | |
| 383 | CTTGTCCAGCTCCTGCCCTGGTTTTAGAGCTA | pegRNA (G-to-T |
| | GAAATAGCAAGTTAAAATAAGGCTAGTCCGTT | nonsynonymous [V47F]; AGG- |
| | ATCAACTTGAAAAAGTGGCACCGAGTCGGTG | to-AGC nonsynonymous |
| | CTTCAATGGCTCAGGGCAGGAGCTGGAACATT | [A49G] PAM silencing edit); |
| | GACGCGTCTCTACGTGGGGGCGCG (SEQ ID | includes linker + hairpin |
| | NO: 383) | |
| 384 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit); includes |
| | ATCAACTTGAAAAAGTGGCACCGAGTCGGTG | linker + hairpin |
| | CTCAATGCGTCAGGGCAGGAGCTGGAAACAT | |
| | TGACGCGTCTCTACGTGGGGGCGCG (SEQ ID | |
| | NO: 384) | |
| 385 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit); includes |
| | ATCAACTTGAAAAAGTGGCACCGAGTCGGTG | linker + hairpin |
| | CGTCAATGCGTCAGGGCAGGAGCTGGAACAT | |
| | TGACGCGTCTCTACGTGGGGGCGCG (SEQ ID | |
| | NO: 385) | |
| 386 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit); includes |
| | ATCAACTTGAAAAAGTGGCACCGAGTCGGTG | linker + hairpin |
| | CTCGTCAATGCGTCAGGGCAGGAGCTAACATT | |
| | GACGCGTCTCTACGTGGGGGCGCG (SEQ ID | |
| | NO: 386) | |
| 387 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CTGCTCGTCAATGCCTCAGGGCAGGAGAACAT | |
| | TGACGCGTCTCTACGTGGGGGCGCG (SEQ ID | |
| | NO: 387) | |
| 388 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CGTCAATGCCTCAGGGCAGGAGCTGGAAACA | |
| | TTGACGCGTCTCTACGTGGGGGCGCG (SEQ ID | |
| | NO: 388) | |
| 389 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CTCGTCAATGCCTCAGGGCAGGAGCTGAACAT | |
| | TGACGCGTCTCTACGTGGGGGCGCG (SEQ ID | |
| | NO: 389) | |
| 390 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CGCTCGTCAATGCCTCAGGGCAGGAGCAACAT | |
| | TGACGCGTCTCTACGTGGGGGCGCG (SEQ ID | |
| | NO: 390) | |
| 391 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CTCAATGCCTCAGGGCAGGAGCTGGACAACA | |
| | TTGACGCGTCTCTACGTGGGGGCGCG (SEQ ID | |
| | NO: 391) | |
| 392 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit); includes linker + |
| | CTCAATGGCTCAGGGCAGGAGCTGGACAACA | hairpin |
| | TTGACGCGTCTCTACGTGGGGGCGCG (SEQ ID | |
| | NO: 392) | |
| 393 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit); includes linker + |
| | CGTCAATGGCTCAGGGCAGGAGCTGGAAACA | hairpin |
| | TTGACGCGTCTCTACGTGGGGGCGCG (SEQ ID | |
| | NO: 393) | |
| 394 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit); includes linker + |
| | CTCGTCAATGGCTCAGGGCAGGAGCTGAACAT | hairpin |
| | TGACGCGTCTCTACGTGGGGGCGCG (SEQ ID | |
| | NO: 394) | |
| 395 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit); includes linker + |
| | CGCTCGTCAATGGCTCAGGGCAGGAGCAACA | hairpin |
| | TTGACGCGTCTCTACGTGGGGGCGCG (SEQ ID | |
| | NO: 395) | |
| 396 | CTTGTCCAGCTCCTGCCCTGGTTTTAGAGCTA | pegRNA (G-to-T |
| | GAAATAGCAAGTTAAAATAAGGCTAGTCCGTT | nonsynonymous [V47F]; AGG- |
| | ATCAACTTGAAAAAGTGGCACCGAGTCGGTG | to-AGC nonsynonymous |
| | CTTCAATGGCTCAGGGCAGGAGCTGGAAACA | [A49G] PAM silencing edit); |
| | TTGACGCGTCTCTACGTGGGGGCGCG (SEQ ID | includes linker + hairpin |
| | NO: 396) | |
| 397 | CTTGTCCAGCTCCTGCCCTGGTTTTAGAGCTA | pegRNA*13 (G-to-T |
| | GAAATAGCAAGTTAAAATAAGGCTAGTCCGTT | nonsynonymous [V47F] edit); |
| | ATCAACTTGAAAAAGTGGCACCGAGTCGGTG | includes linker + hairpin |
| | CTTCAATGCCTCAGGGCAGGAGCTGGAAACAT | |
| | TGACGCGTCTCTACGTGGGGGCGCG (SEQ ID | |
| | NO: 397) | |
| 398 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit); includes |
| | ATCAACTTGAAAAAGTGGCACCGAGTCGGTG | linker + hairpin |
| | CTCAATGCGTCAGGGCAGGAGCTGGACAACA | |
| | TTGACGCGTCTCTACGTGGGGGCGCG (SEQ ID | |
| | NO: 398) | |
| 399 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit); includes |
| | ATCAACTTGAAAAAGTGGCACCGAGTCGGTG | linker + hairpin |
| | CGTCAATGCGTCAGGGCAGGAGCTGGAAACA | |
| | TTGACGCGTCTCTACGTGGGGGCGCG (SEQ ID | |
| | NO: 399) | |
| 400 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit); includes |
| | ATCAACTTGAAAAAGTGGCACCGAGTCGGTG | linker + hairpin |
| | CTCGTCAATGCGTCAGGGCAGGAGCTGAACAT | |
| | TGACGCGTCTCTACGTGGGGGCGCG (SEQ ID | |
| | NO: 400) | |
| 401 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit); includes |
| | ATCAACTTGAAAAAGTGGCACCGAGTCGGTG | linker + hairpin |
| | CGCTCGTCAATGCGTCAGGGCAGGAGCAACA | |
| | TTGACGCGTCTCTACGTGGGGGCGCG (SEQ ID | |
| | NO: 401) | |
| 402 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CTCGTCAATGCCTCAGGGCAGGAGCTGGAAC | |
| | ATTGACGCGTCTCTACGTGGGGGCGCG (SEQ | |
| | ID NO: 402) | |
| 403 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CGTCAATGCCTCAGGGCAGGAGCTGGACAAC | |
| | ATTGACGCGTCTCTACGTGGGGGCGCG (SEQ | |
| | ID NO: 403) | |
| 404 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CGCTCGTCAATGCCTCAGGGCAGGAGCTAACA | |
| | TTGACGCGTCTCTACGTGGGGGCGCG (SEQ ID | |
| | NO: 404) | |
| 405 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CTGCTCGTCAATGCCTCAGGGCAGGAGCAACA | |
| | TTGACGCGTCTCTACGTGGGGGCGCG (SEQ ID | |
| | NO: 405) | |
| 406 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit); includes |
| | ATCAACTTGAAAAAGTGGCACCGAGTCGGTG | linker + hairpin |
| | CTCGTCAATGCGTCAGGGCAGGAGCTGGAAC | |
| | ATTGACGCGTCTCTACGTGGGGGCGCG (SEQ | |
| | ID NO: 406) | |
| 407 | CTTGTCCAGCTCCTGCCCTGGTTTTAGAGCTA | pegRNA*1, 5, 9 (AGG-to-ATG |
| | GAAATAGCAAGTTAAAATAAGGCTAGTCCGTT | PAM silencing edit); includes |
| | ATCAACTTGAAAAAGTGGCACCGAGTCGGTG | linker + hairpin |
| | CTCGTCAATGCATCAGGGCAGGAGCTGGAAC | |
| | ATTGACGCGTCTCTACGTGGGGGCGCG (SEQ | |
| | ID NO: 407) | |
| 408 | CTTGTCCAGCTCCTGCCCTGGTTTTAGAGCTA | pegRNA*4, 8, 12 (AGG-to-AAG |
| | GAAATAGCAAGTTAAAATAAGGCTAGTCCGTT | PAM silencing edit); includes |
| | ATCAACTTGAAAAAGTGGCACCGAGTCGGTG | linker + hairpin |
| | CTCGTCAATGCTTCAGGGCAGGAGCTGGAACA | |
| | TTGACGCGTCTCTACGTGGGGGCGCG (SEQ ID | |
| | NO: 408) | |
| 409 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CTCAATGCCTCAGGGCAGGAGCTGGACAAAC | |
| | ATTGACGCGTCTCTACGTGGGGGCGCG (SEQ | |
| | ID NO: 409) | |
| 410 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit); includes linker + |
| | CTCAATGGCTCAGGGCAGGAGCTGGACAAAC | hairpin |
| | ATTGACGCGTCTCTACGTGGGGGCGCG (SEQ | |
| | ID NO: 410) | |
| 411 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit); includes linker + |
| | CGTCAATGGCTCAGGGCAGGAGCTGGACAAC | hairpin |
| | ATTGACGCGTCTCTACGTGGGGGCGCG (SEQ | |
| | ID NO: 411) | |
| 412 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit); includes linker + |
| | CTCGTCAATGGCTCAGGGCAGGAGCTGGAAC | hairpin |
| | ATTGACGCGTCTCTACGTGGGGGCGCG (SEQ | |
| | ID NO: 412) | |
| 413 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit); includes linker + |
| | CGCTCGTCAATGGCTCAGGGCAGGAGCTAAC | hairpin |
| | ATTGACGCGTCTCTACGTGGGGGCGCG (SEQ | |
| | ID NO: 413) | |
| 414 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit); includes linker + |
| | CTGCTCGTCAATGGCTCAGGGCAGGAGCAAC | hairpin |
| | ATTGACGCGTCTCTACGTGGGGGCGCG (SEQ | |
| | ID NO: 414) | |
| 415 | CTTGTCCAGCTCCTGCCCTGGTTTTAGAGCTA | pegRNA (G-to-T synonymous; |
| | GAAATAGCAAGTTAAAATAAGGCTAGTCCGTT | AGG-to-AGC nonsynonymous |
| | ATCAACTTGAAAAAGTGGCACCGAGTCGGTG | [A49G] PAM silencing edit); |
| | CTCTCGTCAATGGCTCAGGGCAGGAGCTAACA | includes linker + hairpin |
| | TTGACGCGTCTCTACGTGGGGGCGCG (SEQ ID | |
| | NO: 415) | |
| 416 | CTTGTCCAGCTCCTGCCCTGGTTTTAGAGCTA | pegRNA (G-to-T |
| | GAAATAGCAAGTTAAAATAAGGCTAGTCCGTT | nonsynonymous [V47F]; AGG- |
| | ATCAACTTGAAAAAGTGGCACCGAGTCGGTG | to-AGC nonsynonymous |
| | CTTCAATGGCTCAGGGCAGGAGCTGGACAAC | [A49G] PAM silencing edit); |
| | ATTGACGCGTCTCTACGTGGGGGCGCG (SEQ | includes linker + hairpin |
| | ID NO: 416) | |
| 417 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit); includes |
| | ATCAACTTGAAAAAGTGGCACCGAGTCGGTG | linker + hairpin |
| | CTCAATGCGTCAGGGCAGGAGCTGGACAAAC | |
| | ATTGACGCGTCTCTACGTGGGGGCGCG (SEQ | |
| | ID NO: 417) | |
| 418 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit); includes |
| | ATCAACTTGAAAAAGTGGCACCGAGTCGGTG | linker + hairpin |
| | CGTCAATGCGTCAGGGCAGGAGCTGGACAAC | |
| | ATTGACGCGTCTCTACGTGGGGGCGCG (SEQ | |
| | ID NO: 418) | |
| 419 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit); includes |
| | ATCAACTTGAAAAAGTGGCACCGAGTCGGTG | linker + hairpin |
| | CGCTCGTCAATGCGTCAGGGCAGGAGCTAAC | |
| | ATTGACGCGTCTCTACGTGGGGGCGCG (SEQ | |
| | ID NO: 419) | |
| 420 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit); includes |
| | ATCAACTTGAAAAAGTGGCACCGAGTCGGTG | linker + hairpin |
| | CTGCTCGTCAATGCGTCAGGGCAGGAGCAAC | |
| | ATTGACGCGTCTCTACGTGGGGGCGCG (SEQ | |
| | ID NO: 420) | |
| 421 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CTGCTCGTCAATGCCTCAGGGCAGGAGCTAAC | |
| | ATTGACGCGTCTCTACGTGGGGGCGCG (SEQ | |
| | ID NO: 421) | |
| 422 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CGTCAATGCCTCAGGGCAGGAGCTGGACAAA | |
| | CATTGACGCGTCTCTACGTGGGGGCGCG (SEQ | |
| | ID NO: 422) | |
| 423 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CTCGTCAATGCCTCAGGGCAGGAGCTGGAAA | |
| | CATTGACGCGTCTCTACGTGGGGGCGCG (SEQ | |
| | ID NO: 423) | |
| 424 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CGCTCGTCAATGCCTCAGGGCAGGAGCTGAAC | |
| | ATTGACGCGTCTCTACGTGGGGGCGCG (SEQ | |
| | ID NO: 424) | |
| 425 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CTCTGCTCGTCAATGCCTCAGGGCAGGAGAAC | |
| | ATTGACGCGTCTCTACGTGGGGGCGCG (SEQ | |
| | ID NO: 425) | |
| 426 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit); includes linker + |
| | CGTCAATGGCTCAGGGCAGGAGCTGGACAAA | hairpin |
| | CATTGACGCGTCTCTACGTGGGGGCGCG (SEQ | |
| | ID NO: 426) | |
| 427 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit); includes linker + |
| | CTCGTCAATGGCTCAGGGCAGGAGCTGGAAA | hairpin |
| | CATTGACGCGTCTCTACGTGGGGGCGCG (SEQ | |
| | ID NO: 427) | |
| 428 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit); includes linker + |
| | CGCTCGTCAATGGCTCAGGGCAGGAGCTGAA | hairpin |
| | CATTGACGCGTCTCTACGTGGGGGCGCG (SEQ | |
| | ID NO: 428) | |
| 429 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit); includes linker + |
| | CTGCTCGTCAATGGCTCAGGGCAGGAGCTAAC | hairpin |
| | ATTGACGCGTCTCTACGTGGGGGCGCG (SEQ | |
| | ID NO: 429) | |
| 430 | CTTGTCCAGCTCCTGCCCTGGTTTTAGAGCTA | pegRNA (G-to-T synonymous; |
| | GAAATAGCAAGTTAAAATAAGGCTAGTCCGTT | AGG-to-AGC nonsynonymous |
| | ATCAACTTGAAAAAGTGGCACCGAGTCGGTG | [A49G] PAM silencing edit); |
| | CTCTCGTCAATGGCTCAGGGCAGGAGCTGAAC | includes linker + hairpin |
| | ATTGACGCGTCTCTACGTGGGGGCGCG (SEQ | |
| | ID NO: 430) | |
| 431 | CTTGTCCAGCTCCTGCCCTGGTTTTAGAGCTA | pegRNA (G-to-T |
| | GAAATAGCAAGTTAAAATAAGGCTAGTCCGTT | nonsynonymous [V47F]; AGG- |
| | ATCAACTTGAAAAAGTGGCACCGAGTCGGTG | to-AGC nonsynonymous |
| | CTTCAATGGCTCAGGGCAGGAGCTGGACAAA | [A49G] PAM silencing edit); |
| | CATTGACGCGTCTCTACGTGGGGGCGCG (SEQ | includes linker + hairpin |
| | ID NO: 431) | |
| 432 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit); includes |
| | ATCAACTTGAAAAAGTGGCACCGAGTCGGTG | linker + hairpin |
| | CGTCAATGCGTCAGGGCAGGAGCTGGACAAA | |
| | CATTGACGCGTCTCTACGTGGGGGCGCG (SEQ | |
| | ID NO: 432) | |
| 433 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit); includes |
| | ATCAACTTGAAAAAGTGGCACCGAGTCGGTG | linker + hairpin |
| | CTCGTCAATGCGTCAGGGCAGGAGCTGGAAA | |
| | CATTGACGCGTCTCTACGTGGGGGCGCG (SEQ | |
| | ID NO: 433) | |
| 434 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit); includes |
| | ATCAACTTGAAAAAGTGGCACCGAGTCGGTG | linker + hairpin |
| | CGCTCGTCAATGCGTCAGGGCAGGAGCTGAA | |
| | CATTGACGCGTCTCTACGTGGGGGCGCG (SEQ | |
| | ID NO: 434) | |
| 435 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit); includes |
| | ATCAACTTGAAAAAGTGGCACCGAGTCGGTG | linker + hairpin |
| | CTGCTCGTCAATGCGTCAGGGCAGGAGCTAAC | |
| | ATTGACGCGTCTCTACGTGGGGGCGCG (SEQ | |
| | ID NO: 435) | |
| 436 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CTCGTCAATGCCTCAGGGCAGGAGCTGGACA | |
| | ACATTGACGCGTCTCTACGTGGGGGCGCG | |
| | (SEQ ID NO: 436) | |
| 437 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CGCTCGTCAATGCCTCAGGGCAGGAGCTGGA | |
| | ACATTGACGCGTCTCTACGTGGGGGCGCG | |
| | (SEQ ID NO: 437) | |
| 438 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CTGCTCGTCAATGCCTCAGGGCAGGAGCTGAA | |
| | CATTGACGCGTCTCTACGTGGGGGCGCG (SEQ | |
| | ID NO: 438) | |
| 439 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CTCTGCTCGTCAATGCCTCAGGGCAGGAGCAA | |
| | CATTGACGCGTCTCTACGTGGGGGCGCG (SEQ | |
| | ID NO: 439) | |
| 440 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CGTCAATGCCTCAGGGCAGGAGCTGGACAAA | |
| | ACATTGACGCGTCTCTACGTGGGGGCGCG | |
| | (SEQ ID NO: 440) | |
| 441 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit); includes linker + |
| | CTCGTCAATGGCTCAGGGCAGGAGCTGGACA | hairpin |
| | ACATTGACGCGTCTCTACGTGGGGGCGCG | |
| | (SEQ ID NO: 441) | |
| 442 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit); includes linker + |
| | CGCTCGTCAATGGCTCAGGGCAGGAGCTGGA | hairpin |
| | ACATTGACGCGTCTCTACGTGGGGGCGCG | |
| | (SEQ ID NO: 442) | |
| 443 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit); includes linker + |
| | CTGCTCGTCAATGGCTCAGGGCAGGAGCTGAA | hairpin |
| | CATTGACGCGTCTCTACGTGGGGGCGCG (SEQ | |
| | ID NO: 443) | |
| 444 | CTTGTCCAGCTCCTGCCCTGGTTTTAGAGCTA | pegRNA (G-to-T synonymous; |
| | GAAATAGCAAGTTAAAATAAGGCTAGTCCGTT | AGG-to-AGC nonsynonymous |
| | ATCAACTTGAAAAAGTGGCACCGAGTCGGTG | [A49G] PAM silencing edit); |
| | CTCTCGTCAATGGCTCAGGGCAGGAGCTGGAA | includes linker + hairpin |
| | CATTGACGCGTCTCTACGTGGGGGCGCG (SEQ | |
| | ID NO: 444) | |
| 445 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit); includes |
| | ATCAACTTGAAAAAGTGGCACCGAGTCGGTG | linker + hairpin |
| | CTCGTCAATGCGTCAGGGCAGGAGCTGGACA | |
| | ACATTGACGCGTCTCTACGTGGGGGCGCG | |
| | (SEQ ID NO: 445) | |
| 446 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit); includes |
| | ATCAACTTGAAAAAGTGGCACCGAGTCGGTG | linker + hairpin |
| | CGCTCGTCAATGCGTCAGGGCAGGAGCTGGA | |
| | ACATTGACGCGTCTCTACGTGGGGGCGCG | |
| | (SEQ ID NO: 446) | |
| 447 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit); includes |
| | ATCAACTTGAAAAAGTGGCACCGAGTCGGTG | linker + hairpin |
| | CTGCTCGTCAATGCGTCAGGGCAGGAGCTGAA | |
| | CATTGACGCGTCTCTACGTGGGGGCGCG (SEQ | |
| | ID NO: 447) | |
| 448 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CGCTCTGCTCGTCAATGCCTCAGGGCAGGAGA | |
| | ACATTGACGCGTCTCTACGTGGGGGCGCG | |
| | (SEQ ID NO: 448) | |
| 449 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CTGCTCGTCAATGCCTCAGGGCAGGAGCTGGA | |
| | ACATTGACGCGTCTCTACGTGGGGGCGCG | |
| | (SEQ ID NO: 449) | |
| 450 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CTCGTCAATGCCTCAGGGCAGGAGCTGGACA | |
| | AACATTGACGCGTCTCTACGTGGGGGCGCG | |
| | (SEQ ID NO: 450) | |
| 451 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CGCTCGTCAATGCCTCAGGGCAGGAGCTGGA | |
| | AACATTGACGCGTCTCTACGTGGGGGCGCG | |
| | (SEQ ID NO: 451) | |
| 452 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CTCTGCTCGTCAATGCCTCAGGGCAGGAGCTA | |
| | ACATTGACGCGTCTCTACGTGGGGGCGCG | |
| | (SEQ ID NO: 452) | |
| 453 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit); includes linker + |
| | CTCGTCAATGGCTCAGGGCAGGAGCTGGACA | hairpin |
| | AACATTGACGCGTCTCTACGTGGGGGCGCG | |
| | (SEQ ID NO: 453) | |
| 454 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit); includes linker + |
| | CGCTCGTCAATGGCTCAGGGCAGGAGCTGGA | hairpin |
| | AACATTGACGCGTCTCTACGTGGGGGCGCG | |
| | (SEQ ID NO: 454) | |
| 455 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit); includes linker + |
| | CTGCTCGTCAATGGCTCAGGGCAGGAGCTGGA | hairpin |
| | ACATTGACGCGTCTCTACGTGGGGGCGCG | |
| | (SEQ ID NO: 455) | |
| 456 | CTTGTCCAGCTCCTGCCCTGGTTTTAGAGCTA | pegRNA (G-to-T synonymous; |
| | GAAATAGCAAGTTAAAATAAGGCTAGTCCGTT | AGG-to-AGC nonsynonymous |
| | ATCAACTTGAAAAAGTGGCACCGAGTCGGTG | [A49G] PAM silencing edit); |
| | CTCTCGTCAATGGCTCAGGGCAGGAGCTGGAA | includes linker + hairpin |
| | ACATTGACGCGTCTCTACGTGGGGGCGCG | |
| | (SEQ ID NO: 456) | |
| 457 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit); includes |
| | ATCAACTTGAAAAAGTGGCACCGAGTCGGTG | linker + hairpin |
| | CTCGTCAATGCGTCAGGGCAGGAGCTGGACA | |
| | AACATTGACGCGTCTCTACGTGGGGGCGCG | |
| | (SEQ ID NO: 457) | |
| 458 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit); includes |
| | ATCAACTTGAAAAAGTGGCACCGAGTCGGTG | linker + hairpin |
| | CGCTCGTCAATGCGTCAGGGCAGGAGCTGGA | |
| | AACATTGACGCGTCTCTACGTGGGGGCGCG | |
| | (SEQ ID NO: 458) | |
| 459 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit); includes |
| | ATCAACTTGAAAAAGTGGCACCGAGTCGGTG | linker + hairpin |
| | CTGCTCGTCAATGCGTCAGGGCAGGAGCTGGA | |
| | ACATTGACGCGTCTCTACGTGGGGGCGCG | |
| | (SEQ ID NO: 459) | |
| 460 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CGCTCGTCAATGCCTCAGGGCAGGAGCTGGAC | |
| | AACATTGACGCGTCTCTACGTGGGGGCGCG | |
| | (SEQ ID NO: 460) | |
| 461 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CTGCTCGTCAATGCCTCAGGGCAGGAGCTGGA | |
| | AACATTGACGCGTCTCTACGTGGGGGCGCG | |
| | (SEQ ID NO: 461) | |
| 462 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CTCTGCTCGTCAATGCCTCAGGGCAGGAGCTG | |
| | AACATTGACGCGTCTCTACGTGGGGGCGCG | |
| | (SEQ ID NO: 462) | |
| 463 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CGCTCTGCTCGTCAATGCCTCAGGGCAGGAGC | |
| | AACATTGACGCGTCTCTACGTGGGGGCGCG | |
| | (SEQ ID NO: 463) | |
| 464 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CTCGTCAATGCCTCAGGGCAGGAGCTGGACA | |
| | AAACATTGACGCGTCTCTACGTGGGGGCGCG | |
| | (SEQ ID NO: 464) | |
| 465 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit); includes linker + |
| | CGCTCGTCAATGGCTCAGGGCAGGAGCTGGA | hairpin |
| | CAACATTGACGCGTCTCTACGTGGGGGCGCG | |
| | (SEQ ID NO: 465) | |
| 466 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit); includes linker + |
| | CTGCTCGTCAATGGCTCAGGGCAGGAGCTGGA | hairpin |
| | AACATTGACGCGTCTCTACGTGGGGGCGCG | |
| | (SEQ ID NO: 466) | |
| 467 | CTTGTCCAGCTCCTGCCCTGGTTTTAGAGCTA | pegRNA (G-to-T synonymous; |
| | GAAATAGCAAGTTAAAATAAGGCTAGTCCGTT | AGG-to-AGC nonsynonymous |
| | ATCAACTTGAAAAAGTGGCACCGAGTCGGTG | [A49G] PAM silencing edit); |
| | CTCTCGTCAATGGCTCAGGGCAGGAGCTGGAC | includes linker + hairpin |
| | AACATTGACGCGTCTCTACGTGGGGGCGCG | |
| | (SEQ ID NO: 467) | |
| 468 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit); includes |
| | ATCAACTTGAAAAAGTGGCACCGAGTCGGTG | linker + hairpin |
| | CGCTCGTCAATGCGTCAGGGCAGGAGCTGGA | |
| | CAACATTGACGCGTCTCTACGTGGGGGCGCG | |
| | (SEQ ID NO: 468) | |
| 469 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit); includes |
| | ATCAACTTGAAAAAGTGGCACCGAGTCGGTG | linker + hairpin |
| | CTGCTCGTCAATGCGTCAGGGCAGGAGCTGGA | |
| | AACATTGACGCGTCTCTACGTGGGGGCGCG | |
| | (SEQ ID NO: 469) | |
| 470 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CGAGCTCTGCTCGTCAATGCCTCAGGGCAGGA | |
| | GAACATTGACGCGTCTCTACGTGGGGGCGCG | |
| | (SEQ ID NO: 470) | |
| 471 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CGCTCTGCTCGTCAATGCCTCAGGGCAGGAGC | |
| | TAACATTGACGCGTCTCTACGTGGGGGCGCG | |
| | (SEQ ID NO: 471) | |
| 472 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CGCTCGTCAATGCCTCAGGGCAGGAGCTGGAC | |
| | AAACATTGACGCGTCTCTACGTGGGGGCGCG | |
| | (SEQ ID NO: 472) | |
| 473 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CTGCTCGTCAATGCCTCAGGGCAGGAGCTGGA | |
| | CAACATTGACGCGTCTCTACGTGGGGGCGCG | |
| | (SEQ ID NO: 473) | |
| 474 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CTCTGCTCGTCAATGCCTCAGGGCAGGAGCTG | |
| | GAACATTGACGCGTCTCTACGTGGGGGCGCG | |
| | (SEQ ID NO: 474) | |
| 475 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit); includes linker + |
| | CGCTCGTCAATGGCTCAGGGCAGGAGCTGGA | hairpin |
| | CAAACATTGACGCGTCTCTACGTGGGGGCGCG | |
| | (SEQ ID NO: 475) | |
| 476 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit); includes linker + |
| | CTGCTCGTCAATGGCTCAGGGCAGGAGCTGGA | hairpin |
| | CAACATTGACGCGTCTCTACGTGGGGGCGCG | |
| | (SEQ ID NO: 476) | |
| 477 | CTTGTCCAGCTCCTGCCCTGGTTTTAGAGCTA | pegRNA (G-to-T synonymous; |
| | GAAATAGCAAGTTAAAATAAGGCTAGTCCGTT | AGG-to-AGC nonsynonymous |
| | ATCAACTTGAAAAAGTGGCACCGAGTCGGTG | [A49G] PAM silencing edit); |
| | CTCTCGTCAATGGCTCAGGGCAGGAGCTGGAC | includes linker + hairpin |
| | AAACATTGACGCGTCTCTACGTGGGGGCGCG | |
| | (SEQ ID NO: 477) | |
| 478 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit); includes |
| | ATCAACTTGAAAAAGTGGCACCGAGTCGGTG | linker + hairpin |
| | CGCTCGTCAATGCGTCAGGGCAGGAGCTGGA | |
| | CAAACATTGACGCGTCTCTACGTGGGGGCGCG | |
| | (SEQ ID NO: 478) | |
| 479 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit); includes |
| | ATCAACTTGAAAAAGTGGCACCGAGTCGGTG | linker + hairpin |
| | CTGCTCGTCAATGCGTCAGGGCAGGAGCTGGA | |
| | CAACATTGACGCGTCTCTACGTGGGGGCGCG | |
| | (SEQ ID NO: 479) | |
| 480 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CGCTCGTCAATGCCTCAGGGCAGGAGCTGGAC | |
| | AAAACATTGACGCGTCTCTACGTGGGGGCGCG | |
| | (SEQ ID NO: 480) | |
| 481 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CTGCTCGTCAATGCCTCAGGGCAGGAGCTGGA | |
| | CAAACATTGACGCGTCTCTACGTGGGGGCGCG | |
| | (SEQ ID NO: 481) | |
| 482 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CTCTGCTCGTCAATGCCTCAGGGCAGGAGCTG | |
| | GAAACATTGACGCGTCTCTACGTGGGGGCGCG | |
| | (SEQ ID NO: 482) | |
| 483 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CGCTCTGCTCGTCAATGCCTCAGGGCAGGAGC | |
| | TGAACATTGACGCGTCTCTACGTGGGGGCGCG | |
| | (SEQ ID NO: 483) | |
| 484 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CGAGCTCTGCTCGTCAATGCCTCAGGGCAGGA | |
| | GCAACATTGACGCGTCTCTACGTGGGGGCGCG | |
| | (SEQ ID NO: 484) | |
| 485 | CTTGTCCAGCTCCTGCCCTGGTTTTAGAGCTA | pegRNA*14, 15 (AGG-to-AGC |
| | GAAATAGCAAGTTAAAATAAGGCTAGTCCGTT | nonsynonymous [A49G] PAM |
| | ATCAACTTGAAAAAGTGGCACCGAGTCGGTG | silencing edit); includes linker + |
| | CTGCTCGTCAATGGCTCAGGGCAGGAGCTGGA | hairpin |
| | CAAACATTGACGCGTCTCTACGTGGGGGCGCG | |
| | (SEQ ID NO: 485) | |
| 486 | CTTGTCCAGCTCCTGCCCTGGTTTTAGAGCTA | pegRNA*3, 7, 11 (AGG-to-ACG |
| | GAAATAGCAAGTTAAAATAAGGCTAGTCCGTT | PAM silencing edit); includes |
| | ATCAACTTGAAAAAGTGGCACCGAGTCGGTG | linker + hairpin |
| | CTGCTCGTCAATGCGTCAGGGCAGGAGCTGGA | |
| | CAAACATTGACGCGTCTCTACGTGGGGGCGCG | |
| | (SEQ ID NO: 486) | |
| 487 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CGGGAGCTCTGCTCGTCAATGCCTCAGGGCAG | |
| | GAGAACATTGACGCGTCTCTACGTGGGGGCGC | |
| | G (SEQ ID NO: 487) | |
| 488 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CGAGCTCTGCTCGTCAATGCCTCAGGGCAGGA | |
| | GCTAACATTGACGCGTCTCTACGTGGGGGCGC | |
| | G (SEQ ID NO: 488) | |
| 489 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CGCTCTGCTCGTCAATGCCTCAGGGCAGGAGC | |
| | TGGAACATTGACGCGTCTCTACGTGGGGGCGC | |
| | G (SEQ ID NO: 489) | |
| 490 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CTCTGCTCGTCAATGCCTCAGGGCAGGAGCTG | |
| | GACAACATTGACGCGTCTCTACGTGGGGGCGC | |
| | G (SEQ ID NO: 490) | |
| 491 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CTGCTCGTCAATGCCTCAGGGCAGGAGCTGGA | |
| | CAAAACATTGACGCGTCTCTACGTGGGGGCGC | |
| | G (SEQ ID NO: 491) | |
| 492 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CTCTGCTCGTCAATGCCTCAGGGCAGGAGCTG | |
| | GACAAACATTGACGCGTCTCTACGTGGGGGCG | |
| | CG (SEQ ID NO: 492) | |
| 493 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CGCTCTGCTCGTCAATGCCTCAGGGCAGGAGC | |
| | TGGAAACATTGACGCGTCTCTACGTGGGGGCG | |
| | CG (SEQ ID NO: 493) | |
| 494 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CGAGCTCTGCTCGTCAATGCCTCAGGGCAGGA | |
| | GCTGAACATTGACGCGTCTCTACGTGGGGGCG | |
| | CG (SEQ ID NO: 494) | |
| 495 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CGGGAGCTCTGCTCGTCAATGCCTCAGGGCAG | |
| | GAGCAACATTGACGCGTCTCTACGTGGGGGCG | |
| | CG (SEQ ID NO: 495) | |
| 496 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CGGGAGCTCTGCTCGTCAATGCCTCAGGGCAG | |
| | GAGCTAACATTGACGCGTCTCTACGTGGGGGC | |
| | GCG (SEQ ID NO: 496) | |
| 497 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CGAGCTCTGCTCGTCAATGCCTCAGGGCAGGA | |
| | GCTGGAACATTGACGCGTCTCTACGTGGGGGC | |
| | GCG (SEQ ID NO: 497) | |
| 498 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CGCTCTGCTCGTCAATGCCTCAGGGCAGGAGC | |
| | TGGACAACATTGACGCGTCTCTACGTGGGGGC | |
| | GCG (SEQ ID NO: 498) | |
| 499 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CTCTGCTCGTCAATGCCTCAGGGCAGGAGCTG | |
| | GACAAAACATTGACGCGTCTCTACGTGGGGGC | |
| | GCG (SEQ ID NO: 499) | |
| 500 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CGCTCTGCTCGTCAATGCCTCAGGGCAGGAGC | |
| | TGGACAAACATTGACGCGTCTCTACGTGGGGG | |
| | CGCG (SEQ ID NO: 500) | |
| 501 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CGAGCTCTGCTCGTCAATGCCTCAGGGCAGGA | |
| | GCTGGAAACATTGACGCGTCTCTACGTGGGGG | |
| | CGCG (SEQ ID NO: 501) | |
| 502 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CGGGAGCTCTGCTCGTCAATGCCTCAGGGCAG | |
| | GAGCTGGAACATTGACGCGTCTCTACGTGGGG | |
| | GCGCG (SEQ ID NO: 502) | |
| 503 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CGAGCTCTGCTCGTCAATGCCTCAGGGCAGGA | |
| | GCTGGACAACATTGACGCGTCTCTACGTGGGG | |
| | GCGCG (SEQ ID NO: 503) | |
| 504 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CGCTCTGCTCGTCAATGCCTCAGGGCAGGAGC | |
| | TGGACAAAACATTGACGCGTCTCTACGTGGGG | |
| | GCGCG (SEQ ID NO: 504) | |
| 505 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CGAGCTCTGCTCGTCAATGCCTCAGGGCAGGA | |
| | GCTGGACAAACATTGACGCGTCTCTACGTGGG | |
| | GGCGCG (SEQ ID NO: 505) | |
| 506 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CGGGAGCTCTGCTCGTCAATGCCTCAGGGCAG | |
| | GAGCTGGACAACATTGACGCGTCTCTACGTGG | |
| | GGGCGCG (SEQ ID NO: 506) | |
| 507 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CGAGCTCTGCTCGTCAATGCCTCAGGGCAGGA | |
| | GCTGGACAAAACATTGACGCGTCTCTACGTGG | |
| | GGGCGCG (SEQ ID NO: 507) | |
| 508 | CTTGTCCAGCTCCTGCCCTGGTTTTAGAGCTA | pegRNA*2, 6, 10, 13; includes |
| | GAAATAGCAAGTTAAAATAAGGCTAGTCCGTT | linker + hairpin |
| | ATCAACTTGAAAAAGTGGCACCGAGTCGGTG | |
| | CGGGAGCTCTGCTCGTCAATGCCTCAGGGCAG | |
| | GAGCTGGACAAAACATTGACGCGTCTCTACGT | |
| | GGGGGCGCG (SEQ ID NO: 508) | |
| 509 | ACAGTGGCTTTGATCCACAAGTTTTAGAGCTA | PE3 ngRNA (CGG PAM) |
| | GAAATAGCAAGTTAAAATAAGGCTAGTCCGTT | |
| | ATCAACTTGAAAAAGTGGCACCGAGTCGGTG | |
| | C (SEQ ID NO: 509) | |
| 510 | ACATGCAAAACTGAACACTCGTTTTAGAGCTA | PE3 ngRNA (CGG PAM) |
| | GAAATAGCAAGTTAAAATAAGGCTAGTCCGTT | |
| | ATCAACTTGAAAAAGTGGCACCGAGTCGGTG | |
| | C (SEQ ID NO: 510) | |
| 511 | ACTGCCTCACACCCTCTCCGGTTTTAGAGCTA | PE3 ngRNA (TGG PAM) |
| | GAAATAGCAAGTTAAAATAAGGCTAGTCCGTT | |
| | ATCAACTTGAAAAAGTGGCACCGAGTCGGTG | |
| | C (SEQ ID NO: 511) | |
| 512 | AGCCACTGTCCTCTGCAAAAGTTTTAGAGCTA | PE3 ngRNA (CGG PAM) |
| | GAAATAGCAAGTTAAAATAAGGCTAGTCCGTT | |
| | ATCAACTTGAAAAAGTGGCACCGAGTCGGTG | |
| | C (SEQ ID NO: 512) | |
| 513 | ATGCAGTACGGGCTTTTCCAGTTTTAGAGCTA | PE3 ngRNA (CGG PAM) |
| | GAAATAGCAAGTTAAAATAAGGCTAGTCCGTT | |
| | ATCAACTTGAAAAAGTGGCACCGAGTCGGTG | |
| | C (SEQ ID NO: 513) | |
| 514 | CAAGGCCTCAGGGCAGGAGCGTTTTAGAGCT | PE3 ngRNA (TGG PAM) |
| | AGAAATAGCAAGTTAAAATAAGGCTAGTCCG | |
| | TTATCAACTTGAAAAAGTGGCACCGAGTCGGT | |
| | GC (SEQ ID NO: 514) | |
| 515 | CAAGGCTGTCACAACTCCGAGTTTTAGAGCTA | PE3 ngRNA (GGG PAM) |
| | GAAATAGCAAGTTAAAATAAGGCTAGTCCGTT | |
| | ATCAACTTGAAAAAGTGGCACCGAGTCGGTG | |
| | C (SEQ ID NO: 515) | |
| 516 | CAATGCCTCAGGGCAGGAGCGTTTTAGAGCTA | PE3b*13 ngRNA (TGG PAM) |
| | GAAATAGCAAGTTAAAATAAGGCTAGTCCGTT | |
| | ATCAACTTGAAAAAGTGGCACCGAGTCGGTG | |
| | C (SEQ ID NO: 516) | |
| 517 | CAGCCTTGGGGACACCGTTGGTTTTAGAGCTA | PE3 ngRNA (TGG PAM) |
| | GAAATAGCAAGTTAAAATAAGGCTAGTCCGTT | |
| | ATCAACTTGAAAAAGTGGCACCGAGTCGGTG | |
| | C (SEQ ID NO: 517) | |
| 518 | CAGGAGCTGGACAAGTTTATGTTTTAGAGCTA | PE3 ngRNA (GGG PAM) |
| | GAAATAGCAAGTTAAAATAAGGCTAGTCCGTT | |
| | ATCAACTTGAAAAAGTGGCACCGAGTCGGTG | |
| | C (SEQ ID NO: 518) | |
| 519 | CCAAGGCTGTCACAACTCCGGTTTTAGAGCTA | PE3 ngRNA (AGG PAM) |
| | GAAATAGCAAGTTAAAATAAGGCTAGTCCGTT | |
| | ATCAACTTGAAAAAGTGGCACCGAGTCGGTG | |
| | C (SEQ ID NO: 519) | |
| 520 | CCTCGGAGTTGTGACAGCCTGTTTTAGAGCTA | PE3 ngRNA (TGG PAM) |
| | GAAATAGCAAGTTAAAATAAGGCTAGTCCGTT | |
| | ATCAACTTGAAAAAGTGGCACCGAGTCGGTG | |
| | C (SEQ ID NO: 520) | |
| 521 | CTACTTACATGAGAACCGAAGTTTTAGAGCTA | PE3 ngRNA (AGG PAM) |
| | GAAATAGCAAGTTAAAATAAGGCTAGTCCGTT | |
| | ATCAACTTGAAAAAGTGGCACCGAGTCGGTG | |
| | C (SEQ ID NO: 521) | |
| 522 | CTCGGAGTTGTGACAGCCTTGTTTTAGAGCTA | PE3 ngRNA (GGG PAM) |
| | GAAATAGCAAGTTAAAATAAGGCTAGTCCGTT | |
| | ATCAACTTGAAAAAGTGGCACCGAGTCGGTG | |
| | C (SEQ ID NO: 522) | |
| 523 | CTCTGCTCGTCAAGGCCTCAGTTTTAGAGCTA | PE3 ngRNA (GGG PAM) |
| | GAAATAGCAAGTTAAAATAAGGCTAGTCCGTT | |
| | ATCAACTTGAAAAAGTGGCACCGAGTCGGTG | |
| | C (SEQ ID NO: 523) | |
| 524 | CTCTGCTCGTCAATGCCTCAGTTTTAGAGCTA | PE3b*2 ngRNA (GGG PAM) |
| | GAAATAGCAAGTTAAAATAAGGCTAGTCCGTT | |
| | ATCAACTTGAAAAAGTGGCACCGAGTCGGTG | |
| | C (SEQ ID NO: 524) | |
| 525 | CTCTGCTCGTCAATGGCTCAGTTTTAGAGCTA | PE3b*14 ngRNA (GGG PAM) |
| | GAAATAGCAAGTTAAAATAAGGCTAGTCCGTT | |
| | ATCAACTTGAAAAAGTGGCACCGAGTCGGTG | |
| | C (SEQ ID NO: 525) | |
| 526 | GAGAGGGTGTGAGGCAGTGTGTTTTAGAGCTA | PE3 ngRNA (GGG PAM) |
| | GAAATAGCAAGTTAAAATAAGGCTAGTCCGTT | |
| | ATCAACTTGAAAAAGTGGCACCGAGTCGGTG | |
| | C (SEQ ID NO: 526) | |
| 527 | GATCCACAACGGTGTCCCCAGTTTTAGAGCTA | PE3 ngRNA (AGG PAM) |
| | GAAATAGCAAGTTAAAATAAGGCTAGTCCGTT | |
| | ATCAACTTGAAAAAGTGGCACCGAGTCGGTG | |
| | C (SEQ ID NO: 527) | |
| 528 | GCAGGAGCTGGACAAGTTTAGTTTTAGAGCTA | PE3 ngRNA (TGG PAM) |
| | GAAATAGCAAGTTAAAATAAGGCTAGTCCGTT | |
| | ATCAACTTGAAAAAGTGGCACCGAGTCGGTG | |
| | C (SEQ ID NO: 528) | |
| 529 | GCCACTGTCCTCTGCAAAACGTTTTAGAGCTA | PE3 ngRNA (GGG PAM) |
| | GAAATAGCAAGTTAAAATAAGGCTAGTCCGTT | |
| | ATCAACTTGAAAAAGTGGCACCGAGTCGGTG | |
| | C (SEQ ID NO: 529) | |
| 530 | GCTCGTCAAGGCCTCAGGGCGTTTTAGAGCTA | PE3 ngRNA (AGG PAM) |
| | GAAATAGCAAGTTAAAATAAGGCTAGTCCGTT | |
| | ATCAACTTGAAAAAGTGGCACCGAGTCGGTG | |
| | C (SEQ ID NO: 530) | |
| 531 | GCTCGTCAATGCCTCAGGGCGTTTTAGAGCTA | PE3b*6 ngRNA (AGG PAM) |
| | GAAATAGCAAGTTAAAATAAGGCTAGTCCGTT | |
| | ATCAACTTGAAAAAGTGGCACCGAGTCGGTG | |
| | C (SEQ ID NO: 531) | |
| 532 | GCTCGTCAATGGCTCAGGGCGTTTTAGAGCTA | PE3b*15 ngRNA (AGG PAM) |
| | GAAATAGCAAGTTAAAATAAGGCTAGTCCGTT | |
| | ATCAACTTGAAAAAGTGGCACCGAGTCGGTG | |
| | C (SEQ ID NO: 532) | |
| 533 | GCTCTGCTCGTCAAGGCCTCGTTTTAGAGCTA | PE3 ngRNA (AGG PAM) |
| | GAAATAGCAAGTTAAAATAAGGCTAGTCCGTT | |
| | ATCAACTTGAAAAAGTGGCACCGAGTCGGTG | |
| | C (SEQ ID NO: 533) | |
| 534 | GCTCTGCTCGTCAATGCCTCGTTTTAGAGCTA | PE3b*10 ngRNA (AGG PAM) |
| | GAAATAGCAAGTTAAAATAAGGCTAGTCCGTT | |
| | ATCAACTTGAAAAAGTGGCACCGAGTCGGTG | |
| | C (SEQ ID NO: 534) | |
| 535 | GGAGAGGGTGTGAGGCAGTGGTTTTAGAGCT | PE3 ngRNA (TGG PAM) |
| | AGAAATAGCAAGTTAAAATAAGGCTAGTCCG | |
| | TTATCAACTTGAAAAAGTGGCACCGAGTCGGT | |
| | GC (SEQ ID NO: 535) | |
| 536 | GGCAGTGTGGGTTGGGAGCAGTTTTAGAGCTA | PE3 ngRNA (AGG PAM) |
| | GAAATAGCAAGTTAAAATAAGGCTAGTCCGTT | |
| | ATCAACTTGAAAAAGTGGCACCGAGTCGGTG | |
| | C (SEQ ID NO: 536) | |
| 537 | GGGTGTGAGGCAGTGTGGGTGTTTTAGAGCTA | PE3 ngRNA (TGG PAM) |
| | GAAATAGCAAGTTAAAATAAGGCTAGTCCGTT | |
| | ATCAACTTGAAAAAGTGGCACCGAGTCGGTG | |
| | C (SEQ ID NO: 537) | |
| 538 | GGTGTGAGGCAGTGTGGGTTGTTTTAGAGCTA | PE3 ngRNA (GGG PAM) |
| | GAAATAGCAAGTTAAAATAAGGCTAGTCCGTT | |
| | ATCAACTTGAAAAAGTGGCACCGAGTCGGTG | |
| | C (SEQ ID NO: 538) | |
| 539 | GGTTGGGAGCAAGGCCCTTTGTTTTAGAGCTA | PE3 ngRNA (CGG PAM) |
| | GAAATAGCAAGTTAAAATAAGGCTAGTCCGTT | |
| | ATCAACTTGAAAAAGTGGCACCGAGTCGGTG | |
| | C (SEQ ID NO: 539) | |
| 540 | GTACGGGCTTTTCCACGGAGGTTTTAGAGCTA | PE3 ngRNA (AGG PAM) |
| | GAAATAGCAAGTTAAAATAAGGCTAGTCCGTT | |
| | ATCAACTTGAAAAAGTGGCACCGAGTCGGTG | |
| | C (SEQ ID NO: 540) | |
| 541 | TACGGGCTTTTCCACGGAGAGTTTTAGAGCTA | PE3 ngRNA (GGG PAM) |
| | GAAATAGCAAGTTAAAATAAGGCTAGTCCGTT | |
| | ATCAACTTGAAAAAGTGGCACCGAGTCGGTG | |
| | C (SEQ ID NO: 541) | |
| 542 | TACTTACATGAGAACCGAAAGTTTTAGAGCTA | PE3 ngRNA (GGG PAM) |
| | GAAATAGCAAGTTAAAATAAGGCTAGTCCGTT | |
| | ATCAACTTGAAAAAGTGGCACCGAGTCGGTG | |
| | C (SEQ ID NO: 542) | |
| 543 | TCGGAGTTGTGACAGCCTTGGTTTTAGAGCTA | PE3 ngRNA (GGG PAM) |
| | GAAATAGCAAGTTAAAATAAGGCTAGTCCGTT | |
| | ATCAACTTGAAAAAGTGGCACCGAGTCGGTG | |
| | C (SEQ ID NO: 543) | |
| 544 | TTATGGGTGAAATGCAGTACGTTTTAGAGCTA | PE3 ngRNA (GGG PAM) |
| | GAAATAGCAAGTTAAAATAAGGCTAGTCCGTT | |
| | ATCAACTTGAAAAAGTGGCACCGAGTCGGTG | |
| | C (SEQ ID NO: 544) | |
| 545 | TTTATGGGTGAAATGCAGTAGTTTTAGAGCTA | PE3 ngRNA (CGG PAM) |
| | GAAATAGCAAGTTAAAATAAGGCTAGTCCGTT | |
| | ATCAACTTGAAAAAGTGGCACCGAGTCGGTG | |
| | C (SEQ ID NO: 545) | |
| 546 | TTTTCCACGGAGAGGGTGTGGTTTTAGAGCTA | PE3 ngRNA (AGG PAM) |
| | GAAATAGCAAGTTAAAATAAGGCTAGTCCGTT | |
| | ATCAACTTGAAAAAGTGGCACCGAGTCGGTG | |
| | C (SEQ ID NO: 546) | |
| 547 | AGCCACTGTCCTCTGCAAAAGTTTTAGAGCTA | PE3 ngRNA (CGG PAM); |
| | GAAATAGCAAGTTAAAATAAGGCTAGTCCGTT | includes 4 terminal T's |
| | ATCAACTTGAAAAAGTGGCACCGAGTCGGTG | |
| | CTTTT (SEQ ID NO: 547) | |
| 548 | CAATGCCTCAGGGCAGGAGCGTTTTAGAGCTA | PE3b*13 ngRNA (TGG PAM); |
| | GAAATAGCAAGTTAAAATAAGGCTAGTCCGTT | includes 4 terminal T's |
| | ATCAACTTGAAAAAGTGGCACCGAGTCGGTG | |
| | CTTTT (SEQ ID NO: 548) | |
| 549 | CAGCCTTGGGGACACCGTTGGTTTTAGAGCTA | PE3 ngRNA (TGG PAM); |
| | GAAATAGCAAGTTAAAATAAGGCTAGTCCGTT | includes 4 terminal T's |
| | ATCAACTTGAAAAAGTGGCACCGAGTCGGTG | |
| | CTTTT (SEQ ID NO: 549) | |
| 550 | CAGGAGCTGGACAAGTTTATGTTTTAGAGCTA | PE3 ngRNA (GGG PAM); |
| | GAAATAGCAAGTTAAAATAAGGCTAGTCCGTT | includes 4 terminal T's |
| | ATCAACTTGAAAAAGTGGCACCGAGTCGGTG | |
| | CTTTT (SEQ ID NO: 550) | |
| 551 | CCTCGGAGTTGTGACAGCCTGTTTTAGAGCTA | PE3 ngRNA (TGG PAM); |
| | GAAATAGCAAGTTAAAATAAGGCTAGTCCGTT | includes 4 terminal T's |
| | ATCAACTTGAAAAAGTGGCACCGAGTCGGTG | |
| | CTTTT (SEQ ID NO: 551) | |
| 552 | CTCGGAGTTGTGACAGCCTTGTTTTAGAGCTA | PE3 ngRNA (GGG PAM); |
| | GAAATAGCAAGTTAAAATAAGGCTAGTCCGTT | includes 4 terminal T's |
| | ATCAACTTGAAAAAGTGGCACCGAGTCGGTG | |
| | CTTTT (SEQ ID NO: 552) | |
| 553 | CTCTGCTCGTCAATGCCTCAGTTTTAGAGCTA | PE3b*2 ngRNA (GGG PAM); |
| | GAAATAGCAAGTTAAAATAAGGCTAGTCCGTT | includes 4 terminal T's |
| | ATCAACTTGAAAAAGTGGCACCGAGTCGGTG | |
| | CTTTT (SEQ ID NO: 553) | |
| 554 | CTCTGCTCGTCAATGGCTCAGTTTTAGAGCTA | PE3b*14 ngRNA (GGG PAM); |
| | GAAATAGCAAGTTAAAATAAGGCTAGTCCGTT | includes 4 terminal T's |
| | ATCAACTTGAAAAAGTGGCACCGAGTCGGTG | |
| | CTTTT (SEQ ID NO: 554) | |
| 555 | GCAGGAGCTGGACAAGTTTAGTTTTAGAGCTA | PE3 ngRNA (TGG PAM); |
| | GAAATAGCAAGTTAAAATAAGGCTAGTCCGTT | includes 4 terminal T's |
| | ATCAACTTGAAAAAGTGGCACCGAGTCGGTG | |
| | CTTTT (SEQ ID NO: 555) | |
| 556 | GCCACTGTCCTCTGCAAAACGTTTTAGAGCTA | PE3 ngRNA (GGG PAM); |
| | GAAATAGCAAGTTAAAATAAGGCTAGTCCGTT | includes 4 terminal T's |
| | ATCAACTTGAAAAAGTGGCACCGAGTCGGTG | |
| | CTTTT (SEQ ID NO: 556) | |
| 557 | GCTCGTCAATGCCTCAGGGCGTTTTAGAGCTA | PE3b*6 ngRNA (AGG PAM); |
| | GAAATAGCAAGTTAAAATAAGGCTAGTCCGTT | includes 4 terminal T's |
| | ATCAACTTGAAAAAGTGGCACCGAGTCGGTG | |
| | CTTTT (SEQ ID NO: 557) | |
| 558 | GCTCGTCAATGGCTCAGGGCGTTTTAGAGCTA | PE3b*15 ngRNA (AGG PAM); |
| | GAAATAGCAAGTTAAAATAAGGCTAGTCCGTT | includes 4 terminal T's |
| | ATCAACTTGAAAAAGTGGCACCGAGTCGGTG | |
| | CTTTT (SEQ ID NO: 558) | |
| 559 | GCTCTGCTCGTCAATGCCTCGTTTTAGAGCTA | PE3b*10 ngRNA (AGG PAM); |
| | GAAATAGCAAGTTAAAATAAGGCTAGTCCGTT | includes 4 terminal T's |
| | ATCAACTTGAAAAAGTGGCACCGAGTCGGTG | |
| | CTTTT (SEQ ID NO: 559) | |
| 560 | TCGGAGTTGTGACAGCCTTGGTTTTAGAGCTA | PE3 ngRNA (GGG PAM); |
| | GAAATAGCAAGTTAAAATAAGGCTAGTCCGTT | includes 4 terminal T's |
| | ATCAACTTGAAAAAGTGGCACCGAGTCGGTG | |
| | CTTTT (SEQ ID NO: 560) | |
| 561 | TTATGGGTGAAATGCAGTACGTTTTAGAGCTA | PE3 ngRNA (GGG PAM); |
| | GAAATAGCAAGTTAAAATAAGGCTAGTCCGTT | includes 4 terminal T's |
| | ATCAACTTGAAAAAGTGGCACCGAGTCGGTG | |
| | CTTTT (SEQ ID NO: 561) | |
| 562 | TTTATGGGTGAAATGCAGTAGTTTTAGAGCTA | PE3 ngRNA (CGG PAM); |
| | GAAATAGCAAGTTAAAATAAGGCTAGTCCGTT | includes 4 terminal T's |
| | ATCAACTTGAAAAAGTGGCACCGAGTCGGTG | |
| | CTTTT (SEQ ID NO: 562) | |
| 563 | ACAGTGGCTTTGATCCACAAGTTTTAGAGCTA | PE3 ngRNA (CGG PAM); |
| | GAAATAGCAAGTTAAAATAAGGCTAGTCCGTT | includes linker + hairpin |
| | ATCAACTTGAAAAAGTGGCACCGAGTCGGTG | |
| | CAACATTGACGCGTCTCTACGTGGGGGCGCG | |
| | (SEQ ID NO: 563) | |
| 564 | ACATGCAAAACTGAACACTCGTTTTAGAGCTA | PE3 ngRNA (CGG PAM); |
| | GAAATAGCAAGTTAAAATAAGGCTAGTCCGTT | includes linker + hairpin |
| | ATCAACTTGAAAAAGTGGCACCGAGTCGGTG | |
| | CAACATTGACGCGTCTCTACGTGGGGGCGCG | |
| | (SEQ ID NO: 564) | |
| 565 | ACTGCCTCACACCCTCTCCGGTTTTAGAGCTA | PE3 ngRNA (TGG PAM); |
| | GAAATAGCAAGTTAAAATAAGGCTAGTCCGTT | includes linker + hairpin |
| | ATCAACTTGAAAAAGTGGCACCGAGTCGGTG | |
| | CAACATTGACGCGTCTCTACGTGGGGGCGCG | |
| | (SEQ ID NO: 565) | |
| 566 | ATGCAGTACGGGCTTTTCCAGTTTTAGAGCTA | PE3 ngRNA (CGG PAM); |
| | GAAATAGCAAGTTAAAATAAGGCTAGTCCGTT | includes linker + hairpin |
| | ATCAACTTGAAAAAGTGGCACCGAGTCGGTG | |
| | CAACATTGACGCGTCTCTACGTGGGGGCGCG | |
| | (SEQ ID NO: 566) | |
| 567 | CAAGGCCTCAGGGCAGGAGCGTTTTAGAGCT | PE3 ngRNA (TGG PAM); |
| | AGAAATAGCAAGTTAAAATAAGGCTAGTCCG | includes linker + hairpin |
| | TTATCAACTTGAAAAAGTGGCACCGAGTCGGT | |
| | GCAACATTGACGCGTCTCTACGTGGGGGCGCG | |
| | (SEQ ID NO: 567) | |
| 568 | CAAGGCTGTCACAACTCCGAGTTTTAGAGCTA | PE3 ngRNA (GGG PAM); |
| | GAAATAGCAAGTTAAAATAAGGCTAGTCCGTT | includes linker + hairpin |
| | ATCAACTTGAAAAAGTGGCACCGAGTCGGTG | |
| | CAACATTGACGCGTCTCTACGTGGGGGCGCG | |
| | (SEQ ID NO: 568) | |
| 569 | CAGGAGCTGGACAAGTTTATGTTTTAGAGCTA | PE3 ngRNA (GGG PAM); |
| | GAAATAGCAAGTTAAAATAAGGCTAGTCCGTT | includes linker + hairpin |
| | ATCAACTTGAAAAAGTGGCACCGAGTCGGTG | |
| | CAACATTGACGCGTCTCTACGTGGGGGCGCG | |
| | (SEQ ID NO: 569) | |
| 570 | CCAAGGCTGTCACAACTCCGGTTTTAGAGCTA | PE3 ngRNA (AGG PAM); |
| | GAAATAGCAAGTTAAAATAAGGCTAGTCCGTT | includes linker + hairpin |
| | ATCAACTTGAAAAAGTGGCACCGAGTCGGTG | |
| | CAACATTGACGCGTCTCTACGTGGGGGCGCG | |
| | (SEQ ID NO: 570) | |
| 571 | CTACTTACATGAGAACCGAAGTTTTAGAGCTA | PE3 ngRNA (AGG PAM); |
| | GAAATAGCAAGTTAAAATAAGGCTAGTCCGTT | includes linker + hairpin |
| | ATCAACTTGAAAAAGTGGCACCGAGTCGGTG | |
| | CAACATTGACGCGTCTCTACGTGGGGGCGCG | |
| | (SEQ ID NO: 571) | |
| 572 | CTCTGCTCGTCAAGGCCTCAGTTTTAGAGCTA | PE3 ngRNA (GGG PAM); |
| | GAAATAGCAAGTTAAAATAAGGCTAGTCCGTT | includes linker + hairpin |
| | ATCAACTTGAAAAAGTGGCACCGAGTCGGTG | |
| | CAACATTGACGCGTCTCTACGTGGGGGCGCG | |
| | (SEQ ID NO: 572) | |
| 573 | GAGAGGGTGTGAGGCAGTGTGTTTTAGAGCTA | PE3 ngRNA (GGG PAM); |
| | GAAATAGCAAGTTAAAATAAGGCTAGTCCGTT | includes linker + hairpin |
| | ATCAACTTGAAAAAGTGGCACCGAGTCGGTG | |
| | CAACATTGACGCGTCTCTACGTGGGGGCGCG | |
| | (SEQ ID NO: 573) | |
| 574 | GATCCACAACGGTGTCCCCAGTTTTAGAGCTA | PE3 ngRNA (AGG PAM); |
| | GAAATAGCAAGTTAAAATAAGGCTAGTCCGTT | includes linker + hairpin |
| | ATCAACTTGAAAAAGTGGCACCGAGTCGGTG | |
| | CAACATTGACGCGTCTCTACGTGGGGGCGCG | |
| | (SEQ ID NO: 574) | |
| 575 | GCAGGAGCTGGACAAGTTTAGTTTTAGAGCTA | PE3 ngRNA (TGG PAM); |
| | GAAATAGCAAGTTAAAATAAGGCTAGTCCGTT | includes linker + hairpin |
| | ATCAACTTGAAAAAGTGGCACCGAGTCGGTG | |
| | CAACATTGACGCGTCTCTACGTGGGGGCGCG | |
| | (SEQ ID NO: 575) | |
| 576 | GCTCGTCAAGGCCTCAGGGCGTTTTAGAGCTA | PE3 ngRNA (AGG PAM); |
| | GAAATAGCAAGTTAAAATAAGGCTAGTCCGTT | includes linker + hairpin |
| | ATCAACTTGAAAAAGTGGCACCGAGTCGGTG | |
| | CAACATTGACGCGTCTCTACGTGGGGGCGCG | |
| | (SEQ ID NO: 576) | |
| 577 | GCTCTGCTCGTCAAGGCCTCGTTTTAGAGCTA | PE3 ngRNA (AGG PAM); |
| | GAAATAGCAAGTTAAAATAAGGCTAGTCCGTT | includes linker + hairpin |
| | ATCAACTTGAAAAAGTGGCACCGAGTCGGTG | |
| | CAACATTGACGCGTCTCTACGTGGGGGCGCG | |
| | (SEQ ID NO: 577) | |
| 578 | GGAGAGGGTGTGAGGCAGTGGTTTTAGAGCT | PE3 ngRNA (TGG PAM); |
| | AGAAATAGCAAGTTAAAATAAGGCTAGTCCG | includes linker + hairpin |
| | TTATCAACTTGAAAAAGTGGCACCGAGTCGGT | |
| | GCAACATTGACGCGTCTCTACGTGGGGGCGCG | |
| | (SEQ ID NO: 578) | |
| 579 | GGCAGTGTGGGTTGGGAGCAGTTTTAGAGCTA | PE3 ngRNA (AGG PAM); |
| | GAAATAGCAAGTTAAAATAAGGCTAGTCCGTT | includes linker + hairpin |
| | ATCAACTTGAAAAAGTGGCACCGAGTCGGTG | |
| | CAACATTGACGCGTCTCTACGTGGGGGCGCG | |
| | (SEQ ID NO: 579) | |
| 580 | GGGTGTGAGGCAGTGTGGGTGTTTTAGAGCTA | PE3 ngRNA (TGG PAM); |
| | GAAATAGCAAGTTAAAATAAGGCTAGTCCGTT | includes linker + hairpin |
| | ATCAACTTGAAAAAGTGGCACCGAGTCGGTG | |
| | CAACATTGACGCGTCTCTACGTGGGGGCGCG | |
| | (SEQ ID NO: 580) | |
| 581 | GGTGTGAGGCAGTGTGGGTTGTTTTAGAGCTA | PE3 ngRNA (GGG PAM); |
| | GAAATAGCAAGTTAAAATAAGGCTAGTCCGTT | includes linker + hairpin |
| | ATCAACTTGAAAAAGTGGCACCGAGTCGGTG | |
| | CAACATTGACGCGTCTCTACGTGGGGGCGCG | |
| | (SEQ ID NO: 581) | |
| 582 | GGTTGGGAGCAAGGCCCTTTGTTTTAGAGCTA | PE3 ngRNA (CGG PAM); |
| | GAAATAGCAAGTTAAAATAAGGCTAGTCCGTT | includes linker + hairpin |
| | ATCAACTTGAAAAAGTGGCACCGAGTCGGTG | |
| | CAACATTGACGCGTCTCTACGTGGGGGCGCG | |
| | (SEQ ID NO: 582) | |
| 583 | GTACGGGCTTTTCCACGGAGGTTTTAGAGCTA | PE3 ngRNA (AGG PAM); |
| | GAAATAGCAAGTTAAAATAAGGCTAGTCCGTT | includes linker + hairpin |
| | ATCAACTTGAAAAAGTGGCACCGAGTCGGTG | |
| | CAACATTGACGCGTCTCTACGTGGGGGCGCG | |
| | (SEQ ID NO: 583) | |
| 584 | TACGGGCTTTTCCACGGAGAGTTTTAGAGCTA | PE3 ngRNA (GGG PAM); |
| | GAAATAGCAAGTTAAAATAAGGCTAGTCCGTT | includes linker + hairpin |
| | ATCAACTTGAAAAAGTGGCACCGAGTCGGTG | |
| | CAACATTGACGCGTCTCTACGTGGGGGCGCG | |
| | (SEQ ID NO: 584) | |
| 585 | TACTTACATGAGAACCGAAAGTTTTAGAGCTA | PE3 ngRNA (GGG PAM); |
| | GAAATAGCAAGTTAAAATAAGGCTAGTCCGTT | includes linker + hairpin |
| | ATCAACTTGAAAAAGTGGCACCGAGTCGGTG | |
| | CAACATTGACGCGTCTCTACGTGGGGGCGCG | |
| | (SEQ ID NO: 585) | |
| 586 | TTATGGGTGAAATGCAGTACGTTTTAGAGCTA | PE3 ngRNA (GGG PAM); |
| | GAAATAGCAAGTTAAAATAAGGCTAGTCCGTT | includes linker + hairpin |
| | ATCAACTTGAAAAAGTGGCACCGAGTCGGTG | |
| | CAACATTGACGCGTCTCTACGTGGGGGCGCG | |
| | (SEQ ID NO: 586) | |
| 587 | TTTATGGGTGAAATGCAGTAGTTTTAGAGCTA | PE3 ngRNA (CGG PAM); |
| | GAAATAGCAAGTTAAAATAAGGCTAGTCCGTT | includes linker + hairpin |
| | ATCAACTTGAAAAAGTGGCACCGAGTCGGTG | |
| | CAACATTGACGCGTCTCTACGTGGGGGCGCG | |
| | (SEQ ID NO: 587) | |
| 588 | TTTTCCACGGAGAGGGTGTGGTTTTAGAGCTA | PE3 ngRNA (AGG PAM); |
| | GAAATAGCAAGTTAAAATAAGGCTAGTCCGTT | includes linker + hairpin |
| | ATCAACTTGAAAAAGTGGCACCGAGTCGGTG | |
| | CAACATTGACGCGTCTCTACGTGGGGGCGCG | |
| | (SEQ ID NO: 588) | |

### Pharmaceutical Compositions

Disclosed herein are pharmaceutical compositions comprising any of the prime editing composition components, for example, prime editors, fusion proteins, polynucleotides encoding prime editor polypeptides, PEgRNAs, ngRNAs, and/or prime editing complexes described herein.

The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents, e.g., for specific delivery, increasing half-life, or other therapeutic compounds.

In some embodiments, a pharmaceutically-acceptable carrier comprises any vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.)

Formulations of the pharmaceutical compositions described herein can be prepared by any method known or hereafter developed in the art of pharmacology. In general, such preparatory methods include the step of bringing the active ingredient(s) into association with an excipient and/or one or more other accessory ingredients, and then, if necessary and/or desirable, shaping and/or packaging the product into a desired single- or multi-dose unit. Pharmaceutical formulations can additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired.

### Methods of Editing

The methods and compositions disclosed herein can be used to edit a target gene of interest by prime editing.

In some embodiments, the prime editing method comprises contacting a target gene, e.g., a. CLRN1 gene, with a PEgRNA and a prime editor (PE) polypeptide described herein. In some embodiments, the target gene is double stranded, and comprises two strands of DNA complementary to each other. In some embodiments, the contacting with a PEgRNA and the contacting with a prime editor are performed sequentially. In some embodiments, the contacting with a prime editor is performed after the contacting with a PEgRNA. In some embodiments, the contacting with a PEgRNA is performed after the contacting with a prime editor. In some embodiments, the contacting with a PEgRNA, and the contacting with a prime editor are performed simultaneously. In some embodiments, the PEgRNA and the prime editor are associated in a complex prior to contacting a target gene.

In some embodiments, contacting the target gene with the prime editing composition results in binding of the PEgRNA to a target strand of the target gene, e.g., a CLRN1 gene. In some embodiments, contacting the target gene with the prime editing composition results in binding of the PEgRNA to a search target sequence on the target strand of the target gene upon contacting with the PEgRNA. In some embodiments, contacting the target gene with the prime editing composition results in binding of a spacer sequence of the PEgRNA to a search target sequence with the search target sequence on the target strand of the target gene upon said contacting of the PEgRNA.

In some embodiments, contacting the target gene with the prime editing composition results in binding of the prime editor to the target gene, e.g., a CLRN I gene, upon the contacting of the PE composition with the target gene. In some embodiments, the DNA binding domain of the PE associates with the PEgRNA. In sonmc embodiments, the PE binds the target gene, e.g., a CLRN]gene, directed by the PEgRNA. Accordingly, in some embodiments, the contacting of the target gene results in binding of a DNA binding domain of a prime editor of the target gene, e.g., a CLRN1 gene directed by the PEgRNA.

In some embodiments, contacting the target gene with the prime editing composition results in a nick in an edit strand of the target gene, e.g., a CLRN-J gene by the prime editor upon contacting with the target gene, thereby generating a nicked on the edit strand of the target gene. In some embodiments, contacting the target gene with the prime editing composition results in a single-stranded DNA comprising a free 3′ end at the nick site of the edit strand of the target gene. In some embodiments, contacting the target gene with the prime editing composition results in a nick in the edit strand of the target gene by a DNA binding domain of the prime editor, thereby generating a single-stranded DNA comprising a free 3′ end at the nick site. In some embodiments, the DNA binding domain of the prime editor is a Cas domain. In some embodiments, the DNA binding domain of the prime editor is a Cas9. In some embodiments, the DNA binding domain of the prime editor is a Cas9 nickase.

In some embodiments, contacting the target gene with the prime editing composition results in hybridization of the PEgRNA with the 3′ end of the nicked single-stranded DNA, thereby priming DNA polymerization by a DNA polymerase domain of the prime editor. In some embodiments, the free 3′ end of the single-stranded DNA generated at the nick site hybridizes to a primer binding site sequence (PBS) of the contacted PEgRNA, thereby priming DNA polymerization. In some embodiments, the DNA polymerization is reverse transcription catalyzed by a reverse transcriptase domain of the prime editor. In some embodiments, the method comprises contacting the target gene with a DNA polymerase, e.g., a reverse transcriptase, as a part of a prime editor fusion protein or prime editing complex (in cis), or as a separate protein (in trans).

In some embodiments, contacting the target gene with the prime editing composition generates an edited single stranded DNA that is coded by the editing template of the PEgRNA by DNA polymerase mediated polynierization from the 3′ free end of the single-stranded DNA at the nick site. In sonie embodiments, the editing template of the PEgRNA comprises one or more intended nucleotide edits compared to endogenous sequence of the target gene, e.g., a. CLRN1 gene. In some embodiments, the intended nucleotide edits are incorporated in the target gene, by excision of the 5′ single stranded DNA of the edit strand of the target gene generated at the nick site and DNA repair. In some embodiments, the intended nucleotide edits are incorporated in the target gene by excision of the editing target sequence and DNA repair. In some embodiments, excision of the 5′ single stranded DNA of the edit strand generated at the nick site is by a flap endonuclease. In some embodiments, the flap nuclease is FEN1. In some embodiments, the method further comprises contacting the target gene with a flap endonuclease. In some embodiments, the flap endonuclease is provided as a part of a prime editor fusion protein. In some embodiments, the flap endonuclease is provided in trans.

In some embodiments, contacting the target gene with the prime editing composition generates a mismatched heteroduplex comprising the edit strand of the target gene that comprises the edited single stranded DNA, and the unedited target strand of the target gene. Without being bound by theory, the endogenous DNA repair and replication may resolve the mismatched edited DNA to incorporate the nucleotide change(s) to form the desired edited target gene.

In some embodiments, the method further comprises contacting the target gene, e.g., a CLRN1 gene, with a nick guide (ngRNA) disclosed herein. In some embodiments, the ngRNA comprises a spacer that binds a second search target sequence on the edit strand of the target gene. In some embodiments, the contacted ngRNA directs the PE to introduce a nick in the target strand of the target gene. In some embodiments, the nick on the target strand (non-edit strand) results in endogenous DNA repair machinery to use the edit strand to repair the non-edit strand, thereby incorporating the intended nucleotide edit in both strand of the target gene and modifying the target gene. In some embodiments, the ngRNA comprises a spacer sequence that is complementary to, and may hybridize with, the second search target sequence on the edit strand only after the intended nucleotide edit(s) are incorporated in the edit strand of the target gene.

In some embodiments, the target gene is contacted by the ngRNA, the PEgRNA, and the PE simultaneously. In some embodiments, the ngRNA, the PEgRNA, and the PE formi a complex when they contact the target gene. In some embodiments, the target gene is contacted with the ngRNA, the PEgRNA and the prime editor sequentially. In some embodiments, the target gene is contacted with the ngRNA and/or the PEgRNA after contacting the target gene with the PE. In some embodiments, the target gene is contacted with the ngRNA and/or the PEgRNA before contacting the target gene with the prime editor.

In some embodiments, the target gene, e.g., a CLRN1 gene, is in a cell. Accordingly, also provided herein are methods of modifying a cell, such as a human cell, a human primary cell, a human iPSC-derived cell, a human hair cell, a human inner hair cell, a human outer hair cell, a human M0ller cell, and/or a human photoreceptor.

In some embodiments, the prime editing method comprises introducing a PEgRNA, a prime editor, and/or a ngRNA into the cell that has the target gene. In some embodiments, the prime editing method comprises introducing into the cell that has the target gene with a prime editing composition comprising a PEgRNA, a prime editor polypeptide, and/or a ngRNA. In some embodiments, the PEgRNA, the prime editor polypeptide, and/or the ngRNA form a complex prior to the introduction into the cell. In some embodiments, the PEgRNA, the prime editor polypeptide, and/or the ngRNA form a complex after the introduction into the cell. The prime editors, PEgRNA and/or ngRNAs, and prime editing complexes may be introduced into the cell by any delivery approaches described herein or any delivery approach known in the art, including ribonucleoprotein (RNPs), lipid nanoparticles (LNPs), viral vectors, non-viral vectors, mRNA delivery, and physical techniques such as cell membrane disruption by a microfluidics device. The prime editors, PEgRNA and/or ngRNAs, and prime editing complexes may be introduced into the cell simultaneously or sequentially.

In some embodiments, the prime editing method comprises introducing into the cell a PEgRNA or a polynucleotide encoding the PEgRNA, a prime editor polynucleotide encoding a prime editor polypeptide, and optionally an ngRNA or a polynucleotide encoding the ngRNA. In some embodiments, the method comprises introducing the PEgRNA or the polynucleotide encoding the PEgRNA, the polynucleotide encoding the prime editor polypeptide, and/or the ngRNA or the polynucleotide encoding the ngRNA into the cell simultaneously. In some embodiments, the method comprises introducing the PEgR_NA or the polynucleotide encoding the PEgRNA, the polynucleotide encoding the prime editor polypeptide, and/or the ngRNA or the polynucleotide encoding the ngRNA into the cell sequentially. In some embodiments, the method comprises introducing the polynucleotide encoding the prime editor polypeptide into the cell before introduction of the PEgRNA or the polynucleotide encoding the PEgRNA and/or the ngRNA or the polynucleotide encoding the ngRNA. In some embodiments, the polynucleotide encoding the prime editor polypeptide is introduced into and expressed in the cell before introduction of the PEgRNA or the polynucleotide encoding the PEgRNA and/or the ngRNA or the polynucleotide encoding the ngRNA into the cell. In some embodiments, the polynucleotide encoding the prime editor polypeptide is introduced into the cell after the PEgRNA or the polynucleotide encoding the PEgRNA and/or the ngRNA or the polynucleotide encoding the ngRNA are introduced into the cell. The polynucleotide encoding the prime editor polypeptide, the PEgRNA or the polynucleotide encoding the PEgRNA, and/or the ngRNA or the polynucleotide encoding the ngRNA, may be introduced into the cell by any delivery approaches described herein or any delivery approach known in the art, for example, by RNPs, LNPs, viral vectors, non-viral vectors, mRNA delivery, and physical delivery. In some embodiments, the polynucleotide is a DNA polynucleotide. In some embodiments, the polynucleotide is a RNA polynucleotide, e.g., mRNA polynucleotide.

In some embodiments, the polynucleotide encoding the prime editor polypeptide, the polynucleotide encoding the PEgRNA, and/or the polynucleotide encoding the ngRNA integrate into the genome of the cell after being introduced into the cell. In some embodiments, the polynucleotide encoding the prime editor polypeptide, the polynucleotide encoding the PEgRNA, and/or the polynucleotide encoding the ngRNA are introduced into the cell for transient expression. Accordingly, also provided herein are cells modified by prime editing.

In some embodiments, the cell is a eukarvotic cell. In some embodiments, the cell is a mammalian cell. In some embodiments, the cell is a non-human primate cell, bovine cell, porcine cell, rodent or mouse cell. In some embodiments, the cell is a human cell.

In some embodiments, the cell is a primary cell. In some embodiments, the cell is a human primary cell. In some embodiments, the cell is a progenitor cell. In some embodiments, the cell is a stem cell, in some embodiments, the cell is an induced pluripotent stem cell. In some embodiments, the cell is an embryonic stem cell. In some embodiments, the cell is a retinal progenitor cell. In sonme embodiments, the cell is a retina precursor cell. In some embodiments, the cell is a fibroblast.

In some embodiments, the cell is a human progenitor cell. In some embodiments, the cell is a human stem cell, in some embodiments, the cell is an induced human pluripotent stem cell. In some embodiments, the cell is a human embryonic sten cell. In some embodiments, the cell is a human retinal progenitor cell. In some embodiments, the cell is a human retina precursor cell. In some embodiments, the cell is a human fibroblast.

In some embodiments, the cell is a primary cell. In some embodiments, the cell is a human primary cell. In some embodiments, the cell is a retina cell. In some embodiments, the cell is a photoreceptor. In some embodiments, the cell is an inner ear cell. In some embodiments, the cell is a hair cell. In some embodiments, the cell is an inner hair cell. In some embodiments, the cell is an outer hair cell. In some embodiments, the cell is a MOller cell. In some embodiments, the cell is a rod cell. In some embodiments, the cell is a cone cell. In some embodiments, the cell is a human cell from a retina. In some embodiments, the cell is a human photoreceptor. In sonm embodiments, the cell is a human rod cell. In some embodiments, the cell is a human cone cell. In some embodiments, the cell is a human cell from an inner ear. In some embodiments, the cell is a human hair cell. In some embodiments, the cell is an inner hair cell. In some embodiments, the cell is an outer hair cell. In some embodiments, the cell is a Miller cell. In some embodiments, the cell is a primary human photoreceptor∥derived from an induced human pluripotent stem cell (iPSC). In some embodiments, the cell is a primary human hair cell derived from an induced human pluripotent stem cell (iPSC). In some embodiments, the cell is a primary human Miller cell derived from an induced human pluripotent stem cell (iPSC). In some embodiments, the cell is a primary human inner hair cell derived from an induced human pluripotent stem cell (iPSC). In some embodiments, the cell is a primary human outer hair cell derived from an induced human pluripotent stem cell (iPSC).

In some embodiments, the cell is an ex vivo cell. In some embodiments, the cell is an ex vivo cell obtained from a human subject. For example, in some embodiments, the cell is a stem cell, a progenitor cell obtained from a subject having Usher syndrome type 2 disease prior to editing. After correction of the mutation by prime editing, the cell may be administered to the subject. In some embodiments, the cell is in a subject, e.g., a human subject.

In some embodiments, the target gene edited by prime editing is in a chromosome of the cell. In some embodiments, the intended nucleotide edits incorporate in the chromosome of the cell and are inheritable by progeny cells. In some embodiments, the intended nucleotide edits introduced to the cell by the prime editing compositions and methods are such that the cell and progeny of the cell also include the intended nucleotide edits. In some embodiments, the cell is autologous, allogeneic, or xenogeneic to a subject. In some embodiments, the cell is from or derived from a subject. In some embodiments, the cell is from or derived from a human subject. In some embodiments, the cell is introduced back into the subject, e.g., a human subject, after incorporation of the intended nucleotide edits by prime editing.

In some embodiments, the method provided herein comprises introducing the prime editor polypeptide or the polynucleotide encoding the prime editor polypeptide, the PEgRNA or the polynucleotide encoding the PEgRNA, and/or the ngRNA or the polynucleotide encoding the ngRNA into a plurality or a population of cells that comprise the target gene. In some embodiments, the population of cells is of the same cell type. In some embodiments, the population of cells is of the same tissue or organ. In some embodiments, the population of cells is heterogeneous. In some embodiments, the population of cells is homogeneous. In some embodiments, the population of cells is from a single tissue or organ, and the cells are heterogeneous. In some embodiments, the introduction into the population of cells is ex vivo. In some embodiments, the introduction into the population of cells is in vivo, e.g., into a human subject.

In some embodiments, the target gene is in a genome of each cell of the population. In some embodiments, introduction of the prime editor polypeptide or the polynucleotide encoding the p^{r}ime editor polypeptide, the PEgRNA or the polynucleotide encoding the PEgRNA, and/or the ngRNA or the polynucleotide encoding the ngRNA results in incorporation of one or more intended nucleotide edits in the target gene in at least one of the cells in the population of cells. In some embodiments, introduction of the prime editor polypeptide or the polynucleotide encoding the prime editor polypeptide, the PEgRNA or the polynucleotide encoding the PEgRNA, and/or the ngRNA or the polynucleotide encoding the ngRNA results in incorporation of the one or more intended nucleotide edits in the target gene in a plurality of the population of cells. In some embodiments, introduction of the prime editor polypeptide or the polynucleotide encoding the prime editor polypeptide, the PEgRNA or the polynucleotide encoding the PEgRNA, and/or the ngRNA or the polynucleotide encoding the ngRNA results in incorporation of the one or more intended nucleotide edits in the target gene in each cell of the population of cells. In some embodiments, introduction of the prime editor polypeptide or the polynucleotide encoding the prime editor polypeptide, the PEgRNA or the polynucleotide encoding the PEgRNA, and/or the ngRNA or the polynucleotide encoding the ngRNA results in incorporation of the one or more intended nucleotide edits in the target gene in sufficient number of cells such that the disease or disorder is treated, prevented or ameliorated.

In some embodiments, editing efficiency of the prime editing compositions and method described herein can be measured by calculating the percentage of edited target genes in a population of cells introduced with the prime editing composition. In some embodiments, the editing efficiency is determined after 1 hour, 2 hours, 6 hours, 12 hours, 24 hours, 36 hours, 48 hours, 3 days, 4 days, 5 days, 7 days, 10 days, or 14 days of exposing a target gene (e.g., a CLRN1 gene within the genome of a cell) to a prime editing composition. In some embodiments, editing efficiency of the prime editing compositions and method described herein can be measured by calculating the percentage of edited target genes in a population of cells introduced with the prime editing composition. In some embodiments, the editing efficiency is determined after 5, 6, 7, 8, 9, 10, 11, 12, 13, 14, 15, or 16 weeks of exposing a target gene (e.g., a CLRN1 gene within the genome of a cell) to a prime editing composition. In some embodiments, the population of cells introduced with the prime editing composition is ex vivo. In some embodiments, the population of cells introduced with the prime editing composition is in vitro. In some embodiments, the population of cells introduced with the prime editing composition is in vivo. In some embodiments, the prime editing methods disclosed herein have an editing efficiency of at least about 1%, at least about 5%, at least about 10%, at least about 20%, at least about 30%, at least about 40%, at least about 50%, at least about 60%, at least about 70%, at least about 80%, at least about 90%, at least about 95%, or at least about 99% relative to a suitable control. In some embodiments, the prime editing methods disclosed herein have an editing efficiency of at least 25% relative to a suitable control. In some embodiments, the prime editing methods disclosed herein have an editing efficiency of at least 35% relative to a suitable control, prime editing method disclosed herein has an editing efficiency of at least 30% relative to a suitable control. In some embodiments, the prime editing methods disclosed herein have an editing efficiency of at least 45% relative to a suitable control. In some embodiments, the prime editing methods disclosed herein have an editing efficiency of at least 50% relative to a suitable control. In some embodiments, editing efficiency of prime the prime editing compositions and method described herein can be measured by calculating the percentage of edited target genes in a population of cells after in vivo engraftment of the edited cells. In some embodiments, the editing efficiency is determined after 5, 6, 7, 8, 9, 10, 11, 12, 13, 14, 15, or 16 weeks of engraftment. In some embodiments, the editing efficiency is determined after 8 or 16 weeks of engraftment. In some embodiments, prime editing is able to maintain in edited cells at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or more than 95% of editing efficiency after 8 or 16 weeks post engraftment.

In some embodiments, the methods disclosed herein have an editing efficiency of at least about 1%, at least about 5%, at least about 7.5%, at least about 10%, at least about 15%, at least about 20%, at least about 30%, at least about 40%, at least about 50%, at least about 60%, at least about 70%, at least about 80%, at least about 90%, or at least about 95% of editing a primary cell (as measured in a population of primary cells) relative to a suitable control.

In some embodiments, the methods disclosed herein have an editing efficiency of at least about 5%, at least about 7.5%, at least about 10%, at least about 15%, at least about 20%, at least about 30%, at least about 40%, at least about 50%, at least about 60%, at least about 70%, at least about 80%, at least about 90%, or at least about 95% of editing a population of cells (e.g., human primary cell, human iPSC, human fibroblast, human hair cell, human inner hair cell, human outer hair cell, human Müller cell, or human photoreceptor cell) relative to a corresponding control population of cells.

In some embodiments, the prime editing compositions provided herein are capable of incorporated one or more intended nucleotide edits without generating a significant proportion of indels. The term “indel(s)”, as used herein, refers to the insertion or deletion of a nucleotide base within a polynucleotide, for example, a target gene. Such insertions or deletions can lead to frame shift mutations within a coding region of a gene. Indel frequency of editing can be calculated by methods known in the art. In some embodiments, indel frequency can be calculated based on sequence alignment such as the CRISPResso 2 algorithm as described in Clement et al., Nat. Biotechnol. 37(3): 224-226 (2019), which is incorporated herein in its entirety. In some embodiments, the prime editing methods disclosed herein can have an indel frequency of less than 30%, less than 20%, less than 10%, less than 9%, less than 8%, less than 7%, less than 6%, less than 5%, less than 4%, less than 3%, less than 2%, less than 1.5%, or less than 1%. In some embodiments, any number of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a target gene (e.g., a CLRN1 gene within the genome of a cell) to a prime editing composition.

In some embodiments, the prime editing compositions provided herein are capable of incorporated one or more intended nucleotide edits efficiently without generating a significant proportion of indels. In some embodiments, the prime editing methods disclosed herein have an editing efficiency of at least about 1% and an indel frequency of less than 10% in a target cells, e.g., a population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 1% and an indel frequency of less than 7.5% in a population of target cells. In some embodiments, the prime editing methods disclosed herein have an editing efficiency of at least about 1% and an indel frequency of less than 5% in a population of target cells. In some embodiments, the prime editing methods disclosed herein have an editing efficiency of at least about 1% and an indel frequency of less than 2.5% in a population of target cells. In some embodiments, the prime editing methods disclosed herein have an editing efficiency of at least about 1% and an indel frequency of less than 1% in a population of target cells. In some embodiments, the prime editing methods disclosed herein have an editing efficiency of at least about 1% and an indel frequency of less than 0.5% in a population of target cells, e.g., population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 1% and an indel frequency of less than 0.1% in a population of target cells, e.g., a population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 5% and an indel frequency of less than 1% in a population of target cells, e.g., a human primary cells, human iPSC, human fibroblasts, human hair cells, human inner hair cells, human outer hair cells, human Müller cells, or human photoreceptors. In some embodiments, the prime editing methods disclosed herein have an editing efficiency of at least about 5% and an indel frequency of less than 0.5% in a population of target cells, e.g., human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 5% and an indel frequency of less than 0.1% in a population of target cells, e.g., human primary cells, human iPSCs, human fibroblasts, human hair cells, human inner hair cells, human outer hair cells, human Müller cells, or human photoreceptors.

In some embodiments, the prime editing methods disclosed herein have an editing efficiency of at least about 7.5% and an indel frequency of less than 10% in a population of target cells, e.g., human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 7.5% and an indel frequency of less than 7.5% in a population of target cells, e.g., human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 7.5% and an indel frequency of less than 5% in a population of target cells, e.g., a population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 7.5% and an indel frequency of less than 2.5% in a population of target cells, e.g., a population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 7.5% and an indel frequency of less than 1% in a population of target cells, e.g., a a population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 7.5% and an indel frequency of less than 0.5% in a population of target cells, e.g., a population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 7.5% and an indel frequency of less than 0.10% in a population of target cells, e.g., a population of human primary cells, human iPSCs, human fibroblasts, human hair cells, human inner hair cells, human outer hair cells, human Müller cells, or human photoreceptors.

In some embodiments, the prime editing methods disclosed herein have an editing efficiency of at least about 10% and an indel frequency of less than 10% in a population of target cells, e.g., a population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 10% and an indel frequency of less than 7.5% in a population of target cells, e.g., population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 10% and an indel frequency of less than 5% in a population of target cells, e.g., a population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 10% and an indel frequency of less than 2.5% in a population of target cells, e.g., population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 10% and an indel frequency of less than 1% in a in a population of target cells, e.g., population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 10% and an indel frequency of less than 0.5% in a population of target cells, e.g., population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 10% and an indel frequency of less than 0.1% in a population of target cells, e.g., population of human primary cells, human iPSCs, human fibroblasts, human hair cells, human inner hair cells, human outer hair cells, human Müller cells, or human photoreceptors.

In some embodiments, the prime editing methods disclosed herein have an editing efficiency of at least about 15% and an indel frequency of less than 10% in a population of target cells, e.g., population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 15% and an indel frequency of less than 7.5% in a population of target cells, e.g., population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 15% and an indel frequency of less than 5% in a population of target cells, e.g., a population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 15% and an indel frequency of less than 2.5% in a population of target cells, e.g., a population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 15% and an indel frequency of less than 1% in a population of target cells, e.g., a population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 15% and an indel frequency of less than 0.5% in a population of target cells, e.g., a population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 15% and an indel frequency of less than 0.1% in a population of target cells, e.g., a population of human primary cells, human iPSCs, human fibroblasts, human hair cells, human inner hair cells, human outer hair cells, human Müller cells, or human photoreceptors.

In some embodiments, the prime editing methods disclosed herein have an editing efficiency of at least about 20% and an indel frequency of less than 10% in a population of target cells, e.g., population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 20% and an indel frequency of less than 7.5% in a population of target cells, e.g., population of human primary cells, human iPSCs, human fibroblasts, human hair cells, human inner hair cells, human outer hair cells, human M0ller cells, or human photoreceptors. In some embodiments, the prime editing methods disclosed herein have an editing efficiency of at least about 20% and an indel frequency of less than 5% in a population of target cells, e.g., a population of human primary cells, human iPSCs, human fibroblasts, human hair cells, human inner hair cells, human outer hair cells, human M0ller cells, or human photoreceptors. In some embodiments, the prime editing methods disclosed herein have an editing efficiency of at least about 20% and an indel frequency of less than 2.5% in a population of target cells, e.g., a population of human primary cells, human iPSCs, human fibroblasts, human hair cells, human inner hair cells, human outer hair cells, human M0ller cells, or human photoreceptors. In some embodiments, the prime editing methods disclosed herein have an editing efficiency of at least about 20% and an indel frequency of less than 1% in a population of target cells, e.g., a population of human primary cells, human iPSCs, human fibroblasts, human hair cells, human inner hair cells, human outer hair cells, human M0ller cells, or human photoreceptors. In some embodiments, the prime editing methods disclosed herein have an editing efficiency of at least about 20% and an indel frequency of less than 0.5% in a population of target cells, e.g., a population of human primary cells, human iPSCs, human fibroblasts, human hair cells, human inner hair cells, human outer hair cells, human M0ller cells, or human photoreceptors. In some embodiments, the prime editing methods disclosed herein have an editing efficiency of at least about 20% and an indel frequency of less than 0.1% in a population of target cells, e.g., a population of human primary cells, human iPSCs, human fibroblasts, human hair cells, human inner hair cells, human outer hair cells, human M0ller cells, or human photoreceptors.

In some embodiments, the prime editing methods disclosed herein have an editing efficiency of at least about 30% and an indel frequency of less than 10% in a population of target cells, e.g., population of human primary cells, human iPSCs, human fibroblasts, human hair cells, human inner hair cells, human outer hair cells, human M0ller cells, or human photoreceptors. In some embodiments, the prime editing methods disclosed herein have an editing efficiency of at least about 30% and an indel frequency of less than 7.5% in a population of target cells, e.g., population of human primary cells, human iPSCs, human fibroblasts, human hair cells, human inner hair cells, human outer hair cells, human M0ller cells, or human photoreceptors. In some embodiments, the prime editing methods disclosed herein have an editing efficiency of at least about 30% and an indel frequency of less than 5% in a population of target cells, e.g., a population of human primary cells, human iPSCs, human fibroblasts, human hair cells, human inner hair cells, human outer hair cells, human M0ller cells, or human photoreceptors. In some embodiments, the prime editing methods disclosed herein have an editing efficiency of at least about 30% and an indel frequency of less than 2.5% in a population of target cells, e.g., a population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 30% and an indel frequency of less than 1% in a population of target cells, e.g., a population of human primary cells, human iPSCs, human fibroblasts, human hair cells, human inner hair cells, human outer hair cells, human M0ller cells, or human photoreceptors. In some embodiments, the prime editing methods disclosed herein have an editing efficiency of at least about 30% and an indel frequency of less than 0.5% in a population of target cells, e.g., a population of human primary cells, human iPSCs, human fibroblasts, human hair cells, human inner hair cells, human outer hair cells, human M0ller cells, or human photoreceptors. In some embodiments, the prime editing methods disclosed herein have an editing efficiency of at least about 30% and an indel frequency of less than 0.1% in a population of target cells, e.g., a population of human primary cells, human iPSCs, human fibroblasts, human hair cells, human inner hair cells, human outer hair cells, human M0ller cells, or human photoreceptors.

In some embodiments, the prime editing methods disclosed herein have an editing efficiency of at least about 40% and an indel frequency of less than 10% in a population of target cells, e.g., population of human primary cells, human iPSCs, human fibroblasts, human hair cells, human inner hair cells, human outer hair cells, human M0ller cells, or human photoreceptors. In some embodiments, the prime editing methods disclosed herein have an editing efficiency of at least about 40% and an indel frequency of less than 7.5% in a population of target cells, e.g., population of human primary cells, human iPSCs, human fibroblasts, human hair cells, human inner hair cells, human outer hair cells, human M0ller cells, or human photoreceptors. In some embodiments, the prime editing methods disclosed herein have an editing efficiency of at least about 40% and an indel frequency of less than 5% in a population of target cells, e.g., a population of human primary cells, human iPSCs, human fibroblasts, human hair cells, human inner hair cells, human outer hair cells, human M0ller cells, or human photoreceptors. In some embodiments, the prime editing methods disclosed herein have an editing efficiency of at least about 40% and an indel frequency of less than 2.5% in a population of target cells, e.g., a population of human primary cells, human iPSCs, human fibroblasts, human hair cells, human inner hair cells, human outer hair cells, human M0ller cells, or human photoreceptors. In some embodiments, the prime editing methods disclosed herein have an editing efficiency of at least about 40% and an indel frequency of less than 1% in a population of target cells, e.g., a population of human primary cells, human iPSCs, human fibroblasts, human hair cells, human inner hair cells, human outer hair cells, human M0ller cells, or human photoreceptors. In some embodiments, the prime editing methods disclosed herein have an editing efficiency of at least about 40% and an indel frequency of less than 0.5% in a population of target cells, e.g., a population of human primary cells, human iPSCs, human fibroblasts, human hair cells, human inner hair cells, human outer hair cells, human M0ller cells, or human photoreceptors. In some embodiments, the prime editing methods disclosed herein have an editing efficiency of at least about 40% and an indel frequency of less than 0.1% in a population of target cells, e.g., a population of human primary cells, human iPSCs, human fibroblasts, human hair cells, human inner hair cells, human outer hair cells, human M0ller cells, or human photoreceptors.

In some embodiments, the prime editing methods disclosed herein have an editing efficiency of at least about 50% and an indel frequency of less than 10% in a population of target cells, e.g., population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 50% and an indel frequency of less than 7.5% in a population of target cells, e.g., population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 50% and an indel frequency of less than 5% in a population of target cells, e.g., a population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 50% and an indel frequency of less than 2.5% in a population of target cells, e.g., a population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 50% and an indel frequency of less than 1% in a population of target cells, e.g., a population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 50% and an indel frequency of less than 0.5% in a population of target cells, e.g., a population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 50% and an indel frequency of less than 0.1% in a population of target cells, e.g., a population of human primary cells, human iPSCs, human fibroblasts, human hair cells, human inner hair cells, human outer hair cells, human Müller cells, or human photoreceptors.

In some embodiments, the prime editing methods disclosed herein have an editing efficiency of at least about 60% and an indel frequency of less than 10% in a population of target cells, e.g., population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 60% and an indel frequency of less than 7.5% in a population of target cells, e.g., population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 60% and an indel frequency of less than 5% in a population of target cells, e.g., a population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 60% and an indel frequency of less than 2.5% in a population of target cells, e.g., a population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 60% and an indel frequency of less than 1% in a population of target cells, e.g., a population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 60% and an indel frequency of less than 0.5% in a population of target cells, e.g., a population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 60% and an indel frequency of less than 0.1% in a population of target cells, e.g., a population of human primary cells, human iPSCs, human fibroblasts, human hair cells, human inner hair cells, human outer hair cells, human Müller cells, or human photoreceptors.

In some embodiments, the prime editing methods disclosed herein have an editing efficiency of at least about 70% and an indel frequency of less than 10% in a population of target cells, e.g., population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 70% and an indel frequency of less than 7.5% in a population of target cells, e.g., population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 70% and an indel frequency of less than 5% in a population of target cells, e.g., a population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 70% and an indel frequency of less than 2.5% in a population of target cells, e.g., a population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 70% and an indel frequency of less than 1% in a population of target cells, e.g., a population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 70% and an indel frequency of less than 0.5% in a population of target cells, e.g., a population of human primary cells, human iPSCs, human fibroblasts, human hair cells, human inner hair cells, human outer hair cells, human M0ller cells, or human photoreceptors. In some embodiments, the prime editing methods disclosed herein have an editing efficiency of at least about 70% and an indel frequency of less than 0.10% in a population of target cells, e.g., a population of human primary cells, human iPSCs, human fibroblasts, human hair cells, human inner hair cells, human outer hair cells, human M0ller cells, or human photoreceptors.

In some embodiments, the prime editing methods disclosed herein have an editing efficiency of at least about 80% and an indel frequency of less than 10% in a population of target cells, e.g., population of human primary cells, human iPSCs, human fibroblasts, human hair cells, human inner hair cells, human outer hair cells, human M0ller cells, or human photoreceptors. In some embodiments, the prime editing methods disclosed herein have an editing efficiency of at least about 80% and an indel frequency of less than 7.5% in a population of target cells, e.g., population of human primary cells, human iPSCs, human fibroblasts, human hair cells, human inner hair cells, human outer hair cells, human M0ller cells, or human photoreceptors. In some embodiments, the prime editing methods disclosed herein have an editing efficiency of at least about 80% and an indel frequency of less than 5% in a population of target cells, e.g., a population of human primary cells, human iPSCs, human fibroblasts, human hair cells, human inner hair cells, human outer hair cells, human M0ller cells, or human photoreceptors. In some embodiments, the prime editing methods disclosed herein have an editing efficiency of at least about 80% and an indel frequency of less than 2.5% in a population of target cells, e.g., a population of human primary cells, human iPSCs, human fibroblasts, human hair cells, human inner hair cells, human outer hair cells, human M0ller cells, or human photoreceptors. In some embodiments, the prime editing methods disclosed herein have an editing efficiency of at least about 80% and an indel frequency of less than 1% in a population of target cells, e.g., a population of human primary cells, human iPSCs, human fibroblasts, human hair cells, human inner hair cells, human outer hair cells, human M0ller cells, or human photoreceptors. In some embodiments, the prime editing methods disclosed herein have an editing efficiency of at least about 80% and an indel frequency of less than 0.5% in a population of target cells, e.g., a population of human primary cells, human iPSCs, human fibroblasts, human hair cells, human inner hair cells, human outer hair cells, human M0ller cells, or human photoreceptors. In some embodiments, the prime editing methods disclosed herein have an editing efficiency of at least about 80% and an indel frequency of less than 0.10% in a population of target cells, e.g., a population of human primary cells, human iPSCs, human fibroblasts, human hair cells, human inner hair cells, human outer hair cells, human M0ller cells, or human photoreceptors.

In some embodiments, the prime editing methods disclosed herein have an editing efficiency of at least about 90% and an indel frequency of less than 10% in a population of target cells, e.g., population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 90% and an indel frequency of less than 7.5% in a population of target cells, e.g., population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 90% and an indel frequency of less than 5% in a population of target cells, e.g., a population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 90% and an indel frequency of less than 2.5% in a population of target cells, e.g., a population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 90% and an indel frequency of less than 1% in a population of target cells, e.g., a population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 90% and an indel frequency of less than 0.5% in a population of target cells, e.g., a population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 90% and an indel frequency of less than 0.1% in a population of target cells, e.g., a population of human primary cells, human iPSCs, human fibroblasts, human hair cells, human inner hair cells, human outer hair cells, human Müller cells, or human photoreceptors.

In some embodiments, the prime editing methods disclosed herein have an editing efficiency of at least about 95% and an indel frequency of less than 10% in a population of target cells, e.g., population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 95% and an indel frequency of less than 7.5% in a population of target cells, e.g., population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 95% and an indel frequency of less than 5% in a population of target cells, e.g., a population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 95% and an indel frequency of less than 2.5% in a population of target cells, e.g., a population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 95% and an indel frequency of less than 1% in a population of target cells, e.g., a population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 95% and an indel frequency of less than 0.5% in a population of target cells, e.g., a population of human primary cells, human iPSCs, human fibroblasts, human hair cells, human inner hair cells, human outer hair cells, human Müller cells, or human photoreceptors. In some embodiments, the prime editing methods disclosed herein have an editing efficiency of at least about 95% and an indel frequency of less than 0.10% in a population of target cells, e.g., a population of human primary cells, human iPSCs, human fibroblasts, human hair cells, human inner hair cells, human outer hair cells, human Müller cells, or human photoreceptors. In some embodiments, any number of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a target gene (e.g., a CLRN1 gene within the genome of a cell) to a prime editing composition. In some embodiments, the editing efficiency is determined after 1 hour, 2 hours, 6 hours, 12 hours, 24 hours, 36 hours, 48 hours, 3 days, 4 days, 5 days, 7 days, 10 days, or 14 days of exposing a target gene (e.g., a CLRN1 gene within the genome of a cell) to a prime editing composition.

In some embodiments, the prime editing composition described herein result in less than 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% off-target editing in a chromosome that includes the target gene. In some embodiments, off-target editing is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a target gene (e.g., a nucleic acid within the genome of a cell) to a prime editing composition.

In some embodiments, the prime editing compositions (e.g., PEgRNAs and prime editors as described herein) and prime editing methods disclosed herein can be used to edit a target CLRN1 gene. In some embodiments, the target CLRN1 gene comprises a mutation compared to a wild type CLRN1 gene. In some embodiments, the mutation is associated with Usher Syndrome type 3. In some embodiments, the target CLRN1 gene comprises an editing target sequence that contains the mutation associated with Usher Syndrome type 3. In some embodiments, the mutation is in a coding region of the target CLRN1 gene. In some embodiments, the mutation is in an exon of the target CLRN1 gene. In some embodiments, the prime editing method comprises contacting a target CLRN1 gene with a prime editing composition comprising a prime editor, a PEgRNA, and/or a ngRNA. In some embodiments, contacting the target CLRN1 gene with the prime editing composition results in incorporation of one or more intended nucleotide edits in the target CLRN1 gene. In some embodiments, the incorporation is in a region of the target CLRN1 gene that corresponds to an editing target sequence in the CLRN1 gene. In sonm embodiments, the one or more intended nucleotide edits comprises a single nucleotide substitution, an insertion, a deletion, or any combination thereof, compared to the endogenous sequence of the target CLRN1 gene. In some embodiments, incorporation of the one or more intended nucleotide edits results in replacement of one or more mutations within the corresponding sequence that encodes a wild type clarin-1 set forth in SEQ ID NO: 674. In some embodiments, incorporation of the one or more intended nucleotide edits results in replacement of one or more mutations within the corresponding sequence that encodes an isoform of wild type clarin-1, for example, any of SEQ ID Nos.: 676 or 678. In some embodiments, incorporation of the one or more intended nucleotide edits results in r^{e}placement of the one or more mutations within the corresponding sequence in a wild type CLRN1 gene. In some embodiments, incorporation of the one more intended nucleotide edits results in correction of a mutation in the target CLRN1 gene. In some embodiments, the target CLRN I gene comprises an editing target sequence that contains the mutation. In some embodiments, contacting the target CLRN1 gene with the prime editing composition results in incorporation of one or more intended nucleotide edits in the target CLRN1 gene, which corrects the mutation in the editing target sequence (or a double stranded region comprising the editing target sequence and the complementary sequence to the editing target sequence on a target strand) in the target CLRN1 gene. In some embodiments, the mutation is in exon 0 of the target CLRN1 gene. In some embodiments, the mutation results in a c.144T->G nucleotide substitution in the sequence encoding a clarin-I protein and a N48K amino acid substitution in the clarin-1 protein. In some embodiments, the correction results in restoration of wild type expression, i.e., T at position 144 in the sequence encodⁱng the clarin-1 protein, and thereby a restoration of wild type clarin-1 with asparagine at position 48.

In some embodiments, the target CLRN1 gene is in a target cell. Accordingly, in one aspect provided herein is a method of editing a target cell comprising a target CLRN1 gene that encodes a polypeptide that comprises one or more mutations relative to a wild type CLRN1 gene. In some embodiments, the methods of the present disclosure comprise introducing a prime editing composition comprising a PEgRNA, a prime editor polypeptide, and/or a n_{g}RNA into the target cell that has the target CLRN1 gene to edit the target CLRN1 gene, thereby generating an edited cell. In some embodiments, the target cell is a mammalian cell. In some embodiments, the target cell is a human cell. In some embodiments, the target cell is a progenitor cell. In some embodiments, the target cell is a stein cell. In some embodiments, the target cell is an induced pluripotent stem cell. In some embodiments, the target cell is an embryonic stem cell. In some embodiments, the target cell is a retinal progenitor cell. In some embodiments, the target cell is a retina precursor cell. In some embodiments, the target cell is a fibroblast. In some embodiments, the target cell is a human progenitor cell. In some embodiments, the target cell is a human stem cell, in some embodiments, the target cell is an induced human pluripotent stem cell. In some embodiments, the target cell is a human embryonic stem cell. In some embodiments, the target cell is a human retinal progenitor cell. In some embodiments, the target cell is a human retina precursor cell. In some embodiments, the target cell is a human fibroblast. In some embodiments, the target cell is a primary cell. In some embodiments, the target cell is a human primary cell. In some embodiments, the target cell is a retina cell. In some embodiments, the target cell is a photoreceptor. In some embodiments, the target cell is a rod cell. In some embodiments, the target cell is a cone cell. In some embodiments, the target cell is a human cell from a retina. In some embodiments, the target cell is a human photoreceptor. In some embodiments, the target cell is a human MLaller cell. In some embodiments, the target cell is a human rod cell. In some embodiments, the target cell is a human cone cell. In some embodiments, the cell is a human cell from an inner ear. In some embodiments, the cell is a human hair cell. In some embodiments, the cell is a human outer hair cell. In some embodiments, the cell is a human inner hair cell. In some embodiments, the cell is a primary human photoreceptor derived from an induced human pluripotent stem cell (iPSC). In some embodiments, the cell is a primary human hair cell derived from an induced human pluripotent stem cell (iPSC). In some embodiments, the cell is a primary human Müller cell derived from an induced human pluripotent stem cell (iPSC). In some embodiments, the cell is a primary human inner hair cell derived from an induced human pluripotent stem cell (iPSC). In some embodiments, the cell is a primary human outer hair cell derived from an induced human pluripotent stem cell (iPSC). In some embodiments, the target cell is an ex vivo cell. In some embodiments, the target cell is an ex vivo cell obtained from a human subject. In some embodiments, the target cell is in a subject, e.g., a human subject.

In some embodiments, components of a prime editing composition described herein are provided to a target cell in vitro. In some embodiments, components of a prime editing composition described herein are provided to a target cell ex vivo. In some embodiments, components of a prime editing composition described herein are provided to a target cell in vivo.

In some embodiments, incorporation of the one or more intended nucleotide edits in the target CLRN1 gene that comprises one or more mutations restores wild type expression and function of clarin-1 encoded by the CLRN1 gene. In some embodiments, the target CLRN1 gene encodes a N48K amino acid substitution as compared to the wild type clarin-1 CLRN1 protein prior to incorporation of the one or more intended nucleotide edits. In some embodiments, expression and/or function of clarin-1 may be measured when expressed in a target cell. In some embodiments, incorporation of the one or more intended nucleotide edits in the target CLRN1 gene comprising one or more mutations lead to a fold change in a level of CLRN1 gene expression, clarin-1 expression, or a combination thereof. In some embodiments, a change in the level of CLRN1 expression can comprise a fold change of, e.g., 2-fold, 3-fold, 4-fold, 5-fold, 6-fold, 7-fold, 8-fold, 9-fold, 10-fold, 15-fold, 20-fold, 25-fold, 30-fold, 40-fold, 50-fold, 60-fold, 70-fold, 80-fold, 90-fold, 100-fold or greater as compared to expression in a suitable control cell not introduced with a prime editing composition described herein. In some embodiments, incorporation of the one or more intended nucleotide edits in the target CLRN1 gene that comprises one or more mutations restores wild type expression of clarin-1 by at least 10%, 20%, 30%, 40%, 50%, 60%, 70%, 80%, 90%, 95%, 099% or more as compared to wild type expression of the CLRN1 protein in a suitable control cell that comprises a wild type CLRN1 gene.

In some embodiments, a clarin-1 expression increase can be measured by a clarin-1 functional assay. In some embodiments, protein expression can be measured using a protein assay. In some embodiments, protein expression can be measured using antibody testing. In some embodiments, an antibody can comprise anti-clarin-1. In some embodiments, protein expression can be measured using ELISA, mass spectrometry, Western blot, sodium dodecyl sulfate polyacrylamide gel electrophoresis (SDS-PAGE), high performance liquid chromatography (HPLC), electrophoresis, or any combination thereof. In some embodiments, a protein assay can comprise SDS-PAGE and densitometric analysis of a Coomassie Blue-stained gel. Expression and function of clarin-1 protein can be examined ex vivo or in vivo. In some embodiments, clarin-1 protein expression in target cells or target organs, can be examined by, e.g., immunofluorescent assay using an antibody that specifically recognizes clarin-1. In some embodiments, expression and activity can be measured by examining expression and co-localization of proteins involved in the function of clarin-1 protein network. In some embodiments, activity of the clarin-1 protein can be examined in vivo by measuring restoration of retinal function, for example, by visual motor response (VMR) assay. In some embodiments, activity of the clarin-1 protein can be examined in vivo by measuring restoration of hearing function, for example, by investigation of auditory detection, discrimination, identification, and comprehension.

Exemplary Clarin 1 sequences are set forth below.

CLRN1 Variant 1/isoform a protein sequence (SEQ ID NO: 674):
MPSQQKKIIFCMAGVFSFACALGVVTALGTPLWIKATVLCKTGALLVNASGQELDKFMGEM
QYGLFHGEGVRQCGLGARPFRFSFFPDLLKAIPVSIHVNVILFSAILIVLTMVGTAFFMYNAFG
KPFETLHGPLGLYLLSFISGSCGCLVMILFASEVKIHHLSEKIANYKEGTYVYKTQSEKYTTSF
WVIFFCFFVHFLNGLLIRLAGFQFPFAKSKDAETTNVAADLMY
CLRN1 Variant 1/isoform a mRNA/cDNA sequence (SEQ ID NO: 675):
acagaagccgtttctcatcATGccaagccaacagaagaaaatcattttttgcatggccggagtgttcagttttgcatgtgccctcggagttgtgaca
gccttggggacaccgttgtggatcaaagccactgtcctctgcaaaacgggagctctgctcgtcaatgcctcagggcaggagctggacaagtttatg
ggtgaaatgcagtacgggcttttccacggagagggtgtgaggcagtgtgggttgggagcaaggccctttcggttctcattttttccagatttgctcaaa
gcaatcccagtgagcatccacgtcaatgtcattctcttctctgccatccttattgtgttaaccatggtggggacagccttcttcatgtacaatgcttttgga
aaaccttttgaaactctgcatggtcccctagggctgtaccttttgagcttcatttcaggctcctgtggctgtcttgtcatgatattgtttgcctctgaagtg
aaaatccatcacctctcagaaaaaattgcaaattataaagaagggacttatgtctacaaaacgcaaagtgaaaaatataccacctcattctgggtcatttt
cttttgcttttttgttcattttctgaatgggctcctaatacgacttgctggatttcagttcccttttgcaaaatctaaagacgcagaaacaactaatgtag
ctgcagatctaatgtactgaaaggcaaacctttctataattttacaagggagtagacttgctttggtcacttttagatgtggttaattttgcatatccttt
tagtctgcatatattaaagcatcaggacccttcgtgacaatgtttacaaattacgtactaaggatacaggctggaaagtaagggaagcagaaggaaggctt
tgaaaagttgttttatctggtgggaaattgcttgacccaggtagtcaaaggcagttgactagaatcgacaaattgttactccatatatatatatgtgtgtg
tgtgtgtgtgtgtgtgtgtgtaagatgtcttcctatcaaaaagatatcaaaggcacatggaatatattttaataaaaacaaataatatctctaatatatcca
cacatttgttgccagatttcagaaaactgagctgcaatcgctttcctaaaacagtagtgtattaaatgaacatctataaaatgtatcaacacacattttaa
aaaatttgtttaaagtatactcttaggccaggcgtggtgactcacacctgtaattccagcacttcaggaggccaaggtgggaagatcatttgagttcagga
gttcgagttacagcctgggcaataaagtgagaccctgtcactaacaaaattaaaaaataaaataaatataaaatataggctttaaaaaagcatagtcttat
taaccatgtctgttggtcaaaatctgcaaactctaaaagaagaaaagaagaaaaaaccaagcttagggtatttttcctcccgtgcctgagtcccaattaca
ttcacgacagtactttcaatgaacataattgttaggaccactgaggaatcatgaaaaatgatctctgcttagtacatttgatgcaaaatgacttattagggg
ctgtttttctagctatagtgtctcgagtactaatatgcaattatgaaaattatattaaatctgggattatgacggtatcactgtatcatcttggtcttgtt
ctggctgtcaccaagcatgacccaggtcaactttttttttcccctgaattacccatcaaattgatctgcagctgactaaaggccacagctgagcctggaac
tgacccttccttcatcctcaacctgctgtcctccagaaagcaccaaggaaaaagcagagaatgacagcaaacagatcactaggcctctgaccacaggt
gctgagtactcagcagccctcatataataggtttgaaagtactccttaaaataaaacactgtttccctttggaactatttacaaggatgaaacaaccgtat
acctgagaaataacttgctctggtgtcaattcgctattcgccagcagacatcagaacacaccgagtttccagatgct
CLRN1 Variant 5/isoform d protein sequence (SEQ ID NO: 676):
MPSQQKKIIFCMAGVFSFACALGVVTALGTPLWIKATVLCKTGALLVNASGQELDKFMGEM
QYGLFHGEGVRQCGLGARPFRFSFFPDLLKAIPVSIHVNVILFSAILIVLTMVGTAFFMYNAFG
KPFETLHGPLGLYLLSFISVALWLPATRHQAQGSCGCLVMILFASEVKIHHLSEKIANYKEGT
YVYKTQSEKYTTSFWVIFFCFFVHFLNGLLIRLAGFQFPFAKSKDAETTNVAADLMY
CLRN1 Variant 5/isoform d mRNA/cDNA sequence (SEQ ID NO: 677):
aggagatacttgaaggcagtttgaaagacttgttttacagattcttagtccaaagatttccaattagggagaagaagcagcagaaaaggagaaaagc
caagtatgagtgatgatgaggccttcatctactgacatttaacctggcgagaaccgtcgatggtgaagttgccttttcagctgggagctgtccgttcag
cttccgtaataaatgcagtcaaagaggcagtcccttcccattgctcacaaaggtcttgtttttgaacctcgccctcacagaagccgtttctcatcATG
ccaagccaacagaagaaaatcattttttgcatggccggagtgttcagttttgcatgtgccctcggagttgtgacagccttggggacaccgttgtggat
caaagccactgtcctctgcaaaacgggagctctgctcgtcaatgcctcagggcaggagctggacaagtttatgggtgaaatgcagtacgggcttttc
cacggagagggtgtgaggcagtgtgggttgggagcaaggccctttcggttctcattttttccagatttgctcaaagcaatcccagtgagcatccacgt
caatgtcattctcttctctgccatccttattgtgttaaccatggtggggacagccttcttcatgtacaatgcttttggaaaaccttttgaaactctgcatg
gtcccctagggctgtaccttttgagcttcatttcagttgccctttggctgccagctaccaggcaccaggctcaaggctcctgtggctgtcttgtcatgata
ttgtttgcctctgaagtgaaaatccatcacctctcagaaaaaattgcaaattataaagaagggacttatgtctacaaaacgcaaagtgaaaaatataccac
ctcattctgggtcattttcttttgcttttttgttcattttctgaatgggctcctaatacgacttgctggatttcagttcccttttgcaaaatctaaagacg
cagaaacaactaatgtagctgcagatctaatgtactgaaaggcaaacctttctataattttacaagggagtagacttgctttggtcacttttagatgtggt
taattttgcatatccttttagtctgcatatattaaagcatcaggacccttcgtgacaatgtttacaaattacgtactaaggatacaggctggaaagtaagg
gaagcagaaggaaggctttgaaaagttgttttatctggtgggaaattgcttgacccaggtagtcaaaggcagttgactagaatcgacaaattgttactcca
tatatatatatgtgtgtgtgtgtgtgtgtgtgtgtgtgtaagatgtcttcctatcaaaaagatatcaaaggcacatggaatatattttaataaaaacaaat
aatatctctaatatatccacacatttgttgccagatttcagaaaactgagctgcaatcgctttcctaaaacagtagtgtattaaatgaacatctataaaat
gtatcaacacacattttaaaaaatttgtttaaagtatactcttaggccaggcgtggtgactcacacctgtaattccagcacttcaggaggccaaggtggga
agatcatttgagttcaggagttcgagttacagcctgggcaataaagtgagaccctgtcactaacaaaattaaaaaataaaataaatataaaatataggct
ttaaaaaagcatagtcttattaaccatgtctgttggtcaaaatctgcaaactctaaaagaagaaaagaagaaaaaaccaagcttagggtatttttcctcc
cgtgcctgagtcccaattacattcacgacagtactttcaatgaacataattgttaggaccactgaggaatcatgaaaaatgatctctgcttagtacattt
gatgcaaaatgacttattaggggctgtttttctagctatagtgtctcgagtactaatatgcaattatgaaaattatattaaatctgggattatgacggtat
cactgtatcatcttggtcttgttctggctgtcaccaagcatgacccaggtcaactttttttttcccctgaattacccatcaaattgatctgcagctgacta
aaggccacagctgagcctggaactgacccttccttcatcctcaacctgctgtcctccagaaagcaccaaggaaaaagcagagaatgacagcaaacagatca
ctaggcctctgaccacaggtgctgagtactcagcagccctcatataataggtttgaaagtactccttaaaataaaacactgtttccctttggaactattta
caaggatgaaacaaccgtatacctgagaaataacttgctctggtgtcaattcgctattcgccagcagacatcagaacacaccgagtttccagatgct
CLRN1 Variant 6/isoform e protein sequence (SEQ ID NO: 678):
MPSQQKKIIFCMAGVFSFACALGVVTALGTPLWIKATVLCKTGALLVNASGQELDKFMGEM
QYGLFHGEGVRQCGLGARPFRFSCYFLDPFMGLPTGVPHLLSLPCSTSCRREHTSERVQEPAG
CFSAVRSKLHAGPAAATSFSRFAQSNPSEHPRQCHSLLCHPYCVNHGGDSLLHVQCFWKTF
CLRN1 Variant 6/isoform e mRNA/cDNA sequence (SEQ ID NO: 679):
acagaagccgtttctcatcATGccaagccaacagaagaaaatcattttttgcatggccggagtgttcagttttgcatgtgccctcggagttgtgaca
gccttggggacaccgttgtggatcaaagccactgtcctctgcaaaacgggagctctgctcgtcaatgcctcagggcaggagctggacaagtttatg
ggtgaaatgcagtacgggcttttccacggagagggtgtgaggcagtgtgggttgggagcaaggccctttcggttctcatgctattttcttgaccccttc
atgggactcccaacaggggtaccccatttactcagcctgccctgctcaacctcttgcaggagggagcacacgagtgaacgagtgcaggaaccag
ctggctgctttagtgctgtgaggagtaaactccatgcaggccctgcagcagcaaccagtttttccagatttgctcaaagcaatcccagtgagcatcca
cgtcaatgtcattctcttctctgccatccttattgtgttaaccatggtggggacagccttcttcatgtacaatgcttttggaaaaccttttgaaactctgc
atggtcccctagggctgtaccttttgagcttcatttcaggctcctgtggctgtcttgtcatgatattgtttgcctctgaagtgaaaatccatcacctctc
agaaaaaattgcaaattataaagaagggacttatgtctacaaaacgcaaagtgaaaaatataccacctcattctgggtcattttcttttgcttttttgtt
cattttctgaatgggctcctaatacgacttgctggatttcagttcccttttgcaaaatctaaagacgcagaaacaactaatgtagctgcagatctaatgt
actgaaaggcaaacctttctataattttacaagggagtagacttgctttggtcacttttagatgtggttaattttgcatatccttttagtctgcatatatt
aaagcatcaggacccttcgtgacaatgtttacaaattacgtactaaggatacaggctggaaagtaagggaagcagaaggaaggctttgaaaagttgtttta
tctggtgggaaattgcttgacccaggtagtcaaaggcagttgactagaatcgacaaattgttactccatatatatatatgtgtgtgtgtgtgtgtgtgtg
tgtgtgtaagatgtcttcctatcaaaaagatatcaaaggcacatggaatatattttaataaaaacaaataatatctctaatatatccacacatttgttgc
cagatttcagaaaactgagctgcaatcgctttcctaaaacagtagtgtattaaatgaacatctataaaatgtatcaacacacattttaaaaaatttgttta
aagtatactcttaggccaggcgtggtgactcacacctgtaattccagcacttcaggaggccaaggtgggaagatcatttgagttcaggagttcgagttaca
gcctgggcaataaagtgagaccctgtcactaacaaaattaaaaaataaaataaatataaaatataggctttaaaaaagcatagtcttattaaccatgtctg
ttggtcaaaatctgcaaactctaaaagaagaaaagaagaaaaaaccaagcttagggtatttttcctcccgtgcctgagtcccaattacattcacgacagta
ctttcaatgaacataattgttaggaccactgaggaatcatgaaaaatgatctctgcttagtacatttgatgcaaaatgacttattaggggctgtttttcta
gctatagtgtctcgagtactaatatgcaattatgaaaattatattaaatctgggattatgacggtatcactgtatcatcttggtcttgttctggctgtcac
caagcatgacccaggtcaactttttttttcccctgaattacccatcaaattgatctgcagctgactaaaggccacagctgagcctggaactgacccttcct
tcatcctcaacctgctgtcctccagaaagcaccaaggaaaaagcagagaatgacagcaaacagatcactaggcctctgaccacaggtgctgagtactcagc
agccctcatataataggtttgaaagtactccttaaaataaaacactgtttccctttggaactatttacaaggatgaaacaaccgtatacctgagaaataac
ttgctctggtgtcaattcgctattcgccagcagacatcagaacacaccgagtttccagatgct

Methods of Treating Usher Syndrome type 3

In some embodiments, provided herein are methods for treatment of a subject diagnosed with a disease associated with or caused by one or more pathogenic mutations. In some embodiments, provided herein are methods for treatment of a subject diagnosed with a disease associated with or caused by one or more pathogenic mutations that can be corrected by prime editing. In some embodiments, methods of treatment provided herein comprise editing one or more genes other than the gene that harbors the one or more pathogenic mutations. In some embodiments, provided herein are methods for treating Usher Syndrome type 3 that comprise administering to a subject a therapeutically effective amount of a prime editing composition, or a pharmaceutical composition comprising a prime editing composition as described herein. In some embodiments, administration of the prime editing composition results in incorporation of one or more intended nucleotide edits in the target gene in the subject. In some embodiments, administration of the prime editing composition results in correction of one or more pathogenic mutations, e.g., point mutations, insertions, or deletions, associated with Usher Syndrome type 3 in the subject. In some embodiments, administration of the prime editing composition results in correction of one or more pathogenic mutations, e.g., point mutations, insertions, or deletions in the target CLRN1 gene associated with Usher Syndrome type 3 in the subject. In some embodiments, the target gene comprise an editing target sequence that contains the pathogenic mutation. In some embodiments, administration of the prime editing composition results in incorporation of one or more intended nucleotide edits in the target gene that corrects the pathogenic mutation in the editing target sequence (or a double stranded region comprising the editing target sequence and the complementary sequence to the editing target sequence on a target strand) of the target gene in the subject.

In some embodiments, the method provided herein comprises administering to a subject an effective amount of a prime editing composition, for example, a PEgRNA, a prime editor, and/or a ngRNA. In some embodiments, the method comprises administering to the subject an effective amount of a prime editing composition described herein, for example, polynucleotides, vectors, or constructs that encode prime editing composition components, or RNPs, LNPs, and/or polypeptides comprising prime editing composition components. Prime editing compositions can be administered to target the CLRN1 gene in a subject, e.g., a human subject, suffering from, having, susceptible to, or at risk for Usher Syndrome type 3. Identifying a subject in need of such treatment can be in the judgment of a subject or a health care professional and can be subjective (e.g., opinion) or objective (e.g., measurable by a test or diagnostic method). In some embodiments, the subject has Usher Syndrome type 3.

In some embodiments, the subject has been diagnosed with Usher Syndrome type 3 by sequencing of a CLRN1 gene in the subject. In some embodiments, the subject comprises at least a copy of CLRN1 gene that comprises one or more mutations compared to a wild type CLRN1 gene. In some embodiments, the subject comprises at least a copy of CLRN1 gene that comprises a mutation in a coding region of the CLRN1 gene. In some embodiments, the subject comprises at least a copy of CLRN1I gene that comprises a mutation in exon 0, as compared to a wild type CLRN1 gene. In some embodiments, the subject comprises at least a copy of CLRN1 gene that comprises mutation N48K of the CLRN1 gene as compared to a wild type CLRN1 gene.

In some embodiments, the method comprises directly administering prime editing compositions provided herein to a subject. The prime editing compositions described herein can be delivered with in any form as described herein, e.g., as LNPs, RNPs, polynucleotide vectors such as viral vectors, or mRNAs. The prime editing compositions can be formulated with any pharmaceutically acceptable carrier described herein or known in the art for administering directly to a subject. Components of a prime editing composition or a phannaceutical composition thereof may be administered to the subject simultaneously or sequentially. For example, in some embodiments, the method comprises administering a prime editing composition, or pharmaceutical composition thereof, comprising a complex that comprises a prime editor fusion protein and a PEgRNA and/or a ngRNA, to a subject. In some embodiments, the method comprises administering a polynuclcotide or vector encoding a prime editor to a subject simultaneously with a PEgRNA and/or a ngRNA. In some embodiments, the method comprises administering a polynucleotide or vector encoding a prime editor to a subject before administration with a PFgRNA and/or a ngRNA.

Suitable routes of administrating the prime editing compositions to a subject include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration. In some embodiments, the compositions described are administered intraperitoneally, intravenously, or by direct injection or direct infusion. In some embodiments, the compositions described herein are administered by direct injection. In some embodiments, the compositions described herein are administered by subretinal injection. In some embodiments, the compositions described herein are administered by injection to the fovea or parafoveal regions. In some embodiments, the compositions described herein are administered by injection to peripheral regions of the retina. In some embodiments, the compositions described herein are administered by injection through the round window. In some embodiments, the compositions described herein are administered to the retina. In some embodiments, the compositions described herein are administered to a subject by injection, by means of a catheter, by means of a suppository, or by means of an implant.

In some embodiments, the method comprises administering cells edited with a prime editing composition described herein to a subject. In some embodiments, the cells are allogeneic. In some embodiments, allogeneic cells are or have been contacted ex vivo with a prime editing composition or pharmaceutical composition thereof and are introduced into a human subject in need thereof. In some embodiments, the cells are autologous to the subject. In some embodiments, cells are removed from a subject and contacted ex vivo with a prime editing composition or pharmaceutical composition thereof and are re-introduced into the subject.

In some embodiments, cells are contacted ex vivo with one or more components of a prime editing composition. In some embodiments, the ex vivo-contacted cells are introduced into the subject, and the subject is administered in vivo with one or more components of a prime editing composition. For example, in some embodiments, cells are contacted ex vivo with a prime editor and introduced into a subject. In some embodiments, the subject is then administered with a PEgRNA and/or a ngRNA, or a polynucleotide encoding the PEgRNA and/or the ngRNA.

In some embodiments, cells contacted with the prime editing composition are determined for incorporation of the one or more intended nucleotide edits in the genome before re-introduction into the subject. In some embodiments, the cells are enriched for incorporation of the one or more intended nucleotide edits in the genome before re-introduction into the subject. In some embodiments, the edited cells are primary cells. In some embodiments, the edited cells are progenitor cells. In some embodiments, the edited cells are stem cells. In some embodiments, the edited cells are iPSC, fibroblasts, hair cells, inner hair cell, outer hair cells, human Müller cells, or human photoreceptor cells. In some embodiments, the edited cells are primary human cells. In some embodiments, the edited cells are human progenitor cells. In some embodiments, the edited cells are human stem cells. In some embodiments, the edited cells are human iPSC, human fibroblast, human hair cell, human inner hair cell, human outer hair cell, human Müller cell, or human photoreceptor cells. In some embodiments, the cell is a neuron. In some embodiments, the cell is a neuron from basal ganglia. In some embodiments, the cell is a neuron from basal ganglia of a subject. In some embodiments, the cell is a neuron in the basal ganglia of a subject. In some embodiments, the edited cells are an ex vivo cells. In some embodiments, the edited cells are an ex vivo cells obtained from a human subject. In some embodiments, the edited cells are in a subject, e.g., a human subject. The prime editing composition or components thereof may be introduced into a cell by any delivery approaches as described herein, including LNP administration, RNP administration, electroporation, nucleofection, transfection, viral transduction, microinjection, cell membrane disruption and diffusion, or any other approach known in the art.

The cells edited with prime editing can be introduced into the subject by any route known in the art. In some embodiments, the edited cells are administered to a subject by direct infusion. In some embodiments, the edited cells are administered to a subject by intravenous infusion. In some embodiments, the edited cells are administered to a subject as implants.

The pharmaceutical compositions, prime editing compositions, and cells, as described herein, can be administered in effective amounts. In some embodiments, the effective amount depends upon the mode of administration. In some embodiments, the effective amount depends upon the stage of the condition, the age and physical condition of the subject, the nature of concurrent therapy, if any, and like factors well-known to the medical practitioner.

The specific dose administered can be a uniform dose for each subject. Alternatively, a subject's dose can be tailored to the approximate body weight of the subject. Other factors in determining the appropriate dosage can include the disease or condition to be treated or prevented, the severity of the disease, the route of administration, and the age, sex and medical condition of the patient.

In embodiments wherein components of a prime editing composition are administered sequentially, the time between sequential administration can be at least 1 hour, at least 2 hours, at least 6 hours, at least 12 hours, at least 24 hours, at least 36 hours, at least 48 hours, at least³ days, at least 4 days, at least 5 days, at least 7 days, at least 10 days, or at least 14 days.

In some embodiments, a method of monitoring treatiment progress is provided. In some embodiments, the method includes the step of determining a level of diagnostic marker, for example, correction of a mutation in CLRN1 gene, or diagnostic measurement associated with Usher Syndrome type 3, in a subject suffering from Usher Syndrome type 3 symptoms and has been administered an effective amount of a prime editing composition described herein. The level of the diagnostic marker determined in the method can be compared to known levels of the marker in either healthy normal controls or in other afflicted subjects to establish the subject's disease status. Delivery

Prime editing compositions described herein can be delivered to a cellular environment with any approach known in the art. Components of a prime editing composition can be delivered to a cell by the same mode or different modes. For example, in some embodiments, a prime editor can be delivered as a polypeptide or a polynucleotide (DNA or RNA) encoding the polypeptide. In some embodiments, a PEgRNA can be delivered directly as an RNA or as a DNA encoding the PEgRNA.

In some embodiments, a prime editing composition component is encoded by a polynucleotide, a vector, or a construct. In some embodiments, a prime editor polypeptide, a PEgRNA and/or a ngRNA is encoded by a polynucleotide. In some embodiments, the polynucleotide encodes a prime editor fusion protein comprising a DNA binding domain and a DNA polymerase domain. In some embodiments, the polynucleotide encodes a DNA polymerase domain of a prime editor. In some embodiments, the polynucleotide encodes a DNA polymerase domain of a prime editor. In some embodiments, the polynucleotide encodes a portion of a prime editor protein, for example, a N-terminal portion of a prime editor fusion protein connected to an intein-N. In some embodiments, the polynucleotide encodes a portion of a prime editor protein, for example, a C-terminal portion of a prime editor fusion protein connected to an intein-C. In some embodiments, the polynucleotide encodes a PEgRNA and/or a ngRNA. In some embodiments, the polypeptide encodes two or more components of a prime editing composition, for example, a prime editor fusion protein and a PEgRNA.

In some embodiments, the polynucleotide encoding one or more prime editing composition components is delivered to a target cell is integrated into the genome of the cell for long-term expression, for example, by a retroviral vector. In some embodiments, the polynucleotide delivered to a target cell is expressed transiently. For example, the polynucleotide may be delivered in the form of a mRNA, or a non-integrating vector (non-integrating virus, plasmids, minicircle DNAs) for episomal expression.

In some embodiments, a polynucleotide encoding one or more prime editing system components can be operably linked to a regulatory element, e.g., a transcriptional control element, such as a promoter. In some embodiments, the polynucleotide is operably linked to multiple control elements. Depending on the expression system utilized, any of a number of suitable transcription and translation control elements, including constitutive and inducible promoters, transcription enhancer elements, transcription terminators, etc. may be used in the expression vector (e.g., U6 promoter, Hi promoter).

In some embodiments, the polynucleotide encoding one or more prime editing composition components is a part of, or is encoded by, a vector. In some embodim^{e}nts, the vector is a viral vector. In some embodiments, the vector is a non-viral vector.

Non-viral vector delivery systems can include DNA plasmids, RNA (e.g., a transcript of a vector described herein), naked nucleic acid, and nucleic acid complexed with a delivery vehicle, such as a liposome. In some embodiments, the polynucleotide is provided as an RNA. e.g., a mRNA or a transcript. Any RNA of the prime editing systems, for example a guide RNA or a base editor-encoding mRNA, can be delivered in the form of RNA. In some embodiments, one or more components of the prime editing system that are RNAs is produced by direct chemical synthesis or may be transcribed in vitro from a DNA. In some embodiments, a mRNA that encodes a prime editor polypeptide is generated using in vitro transcription. Guide polynucleotides (e.g., PEgRNA or ngRNA) can also be transcribed using in vitro transcription from a cassette containing a T7 promoter, followed by the sequence “GG”, and guide polynucleotide sequence. In some embodiments, the prime editor encoding mRNA PEgRNA, and/or ngRNA are synthesized in vitro using an RNA polymerase enzyme (e.g., T7 polymerase, T3 polymerase, SP6 polymerase, etc.). Once synthesized, the RNA can directly contact a target CLRN1 gene or can be introduced into a cell using any suitable technique for introducing nucleic acids into cells (e.g., microinjection, electroporation, transfection). In some embodiments, the prime editor-coding sequences, the PEgRNAs, and/or the ngRNAs are modified to include one or more modified nucleoside e.g., using pseudo-U or 5-Methyl-C.

Methods of non-viral delivery of nucleic acids can include lipofection, electroporation, nucleofection, microinjection, biolistics, virosomes, liposomes, immunoliposomes, polycation or lipid:nucleic acid conjugates, nanoparticles, cell penetrating peptides and associated conjugated molecules and chemistry, naked DNA, artificial virions, cell membrane disruption by a microfluidics device, and agent-enhanced uptake of DNA. Cationic and neutral lipids that are suitable for efficient receptor-recognition lipofection of polynucleotides can be used. Delivery can be to cells (e.g., in vitro or ex vivo administration) or target tissues (e.g., in vivo administration). The preparation of lipid:nucleic acid complexes, including targeted liposomes such as immunolipid complexes, can be used.

Viral vector delivery systems can include DNA and RNA viruses, which can have either episomal or integrated genomnes after delivery to the cell. RNA or DNA viral based systems can be used to target specific cells and trafficking the viral payload to an organelle of the cell. Viral vectors can be administered directly (in vivo) or they can be used to treat cells in vitro, and the modified cells can optionally be administered after delivery (ex vivo).

In some embodiments, the viral vector is a retroviral, lentiviral, adenoviral, adeno-associated viral or herpes simplex viral vector. Retroviral vectors can include those based upon murine leukemia virus (MuLV), gibbon ape leukemia virus (GaLV), simian immunodeficiency virus (SIV), human immunodeficiency virus (HIV), and combinations thereof. In some embodiments, the retroviral vector is a lentiviral vector. In some embodiments, the retroviral vector is a gamma retroviral vector. In some embodiments, the viral vector is an adenoviral vector. In some embodiments, the viral vector is an adeno-associated virus (“AAV”) vector.

In some embodiments, polynucleotides encoding one or more prime editing composition components are packaged in a virus particle. Packaging cells can be used to form virus particles that can infect a target cell. Such cells can include 293 cells, (e.g., for packaging adenovirus), and psi.2 cells or PA317 cells (e.g., for packaging retrovirus). Viral vectors can be generated by producing a cell line that packages a nucleic acid vector into a viral particle. The vectors can contain the minimal viral sequences required for packaging and subsequent integration into a host. The vectors can contain other viral sequences being replaced by an expression cassette for the polynucleotide(s) to be expressed. The missing viral functions can be supplied in trans by the packaging cell line. For example, AAV vectors can comprise ITR sequences from the AAV genome which are required for packaging and integration into the host genome. In some embodiment, the polynucleotides are a DNA polynucleotide. In some embodiment, the polynucleotides are an RNA polynucleotide; e.g., an mRNA polynucleotide.

In some embodiments, the AAV vector is selected for tropism to a particular cell, tissue, organism. In some embodiments, the AAV vector is pseudotyped, e.g., AAV5/8. In some embodiments, polynucleotides encoding one or more prime editing composition components are packaged in a first AAV and a second AAV. In some embodiments, the polynucleotides encoding one or more prime editing composition components are packaged in a first rAAV and a second rAAV.

In some embodiments, dual AAV vectors are generated by splitting a large transgene expression cassette in two separate halves (5′ and 3′ ends that encode N-terminal portion and C-terminal portion of, e.g., a prime editor polypeptide), where each half of the cassette is no more than 5kb in length, optionally no more than 4.7 kb in length, and is packaged in a single AAV vector. In some embodiments, the full-length transgene expression cassette is reassembled upon co-infection of the same cell by both dual AAV vectors. In some embodiments, a portion or fragment of a prime editor polypeptide, e.g., a Cas9 nickase, is fused to an intein. The portion or fragment of the polypeptide can be fused to the N-terminus or the C-terminus of the intein. In some embodiments, a N-terminal portion of the polypeptide is fused to an intein-N, and a C-terminal portion of the polypeptide is separately fused to an intein-C. In some embodiments, a portion or fragment of a prime editor fusion protein is fused to an intein and fused to an AAV capsid protein. In some embodiments, intein-N may be fused to the N-terminal portion of a first domain described herein, and intein-C may be fused to the C-terminal portion of a second domain described herein for the joining of the N-terminal portion to the C-terminal portion, thereby joining the first and second domains. In some embodiments, the first and second domains are each independently chosen from a DNA binding domain or a DNA polymerase domain. The intein, nuclease and capsid protein can be fused together in any arrangement (e.g., nuclease-intein-capsid, intein-nuclease-capsid, capsid-intein-nuclease, etc.). In some embodiments, a polynucleotide encoding a prime editor fusion protein is split in two separate halves, each encoding a portion of the prime editor fusion protein and separately fused to an intein. In some embodiments, each of the two halves of the polynucleotide is packaged in an individual AAV vector of a dual AAV vector system. In some embodiments, each of the two halves of the polynucleotide is no more than 5kb in length, optionally no more than 4.7 kb in length. In some embodiments, the full-length prime editor fusion protein is reassembled upon co-infection of the same cell by both dual AAV vectors, expression of both halves of the prime editor fusion protein, and self-excision of the inteins. In some embodiments, the in vivo use of dual AAV vectors results in the expression of full-length full-length prime editor fusion proteins. In some embodiments, the use of the dual AAV vector platform allows viable delivery of transgenes of greater than about 4.5, 4.6, 4.7, 4.8, 4.9, or 5.0 kb in size.

In some embodiments, an intein is inserted at a splice site within a Cas protein. In some embodiments, insertion of an intein disrupts a Cas activity. As used herein, “intein” refers to a self-splicing protein intron (e.g., peptide), e.g., which ligates flanking N-terminal and C-terminal exteins (e.g., fragments to be joined). In some embodiments, an intein may comprise a polypeptide that is able to excise itself and join exteins with a peptide bond (e.g., protein splicing). In some embodiments, an intein of a precursor gene comes from two genes (e.g., split intein). In some embodiments, an intein may be a synthetic intein. Non-limiting examples of intein pairs that may be used in accordance with the present disclosure include: dnaE-n and dnaE-c, a 4-hydroxytamoxifen (4-HT)-responsive intein, an iCas molecule, a Ssp DnaX intein, Ter DnaE3 intein, Ter ThyX intein, Rma DnaB intein, Cfa DnaE intein, Ssp GyrB intein, and Rma DnaB intein. In some embodiments, intein fragments may be fused to the N terminal and C-terminal portion of a split Cas protein respectively for joining the fragments of split Cas9.

In some embodiments, the split Cas9 system may be used in general to bypass the packing limit of the viral delivery vehicles. In some embodiments, a split Cas9 may be a Type II CRISPR system Cas9. In some embodiments, a first nucleic acid encodes a first portion of the Cas9 protein having a first split-intein and wherein the second nucleic acid encodes a second portion of the Cas9 protein having a second split-intein complementary to the first split-intein and wherein the first portion of the Cas9 protein and the second portion of the Cas9 protein are joined together to form the Cas9 protein. In some embodiments, the first portion of the Cas9 protein is the N-terminal fragment of the Cas9 protein and the second portion of the Cas9 protein is the C-terminal fragment of the Cas9 protein. In some embodiments, a split site may be selected which are surface exposed due to the sterical need for protein splicing.

In some embodiments, a Cas protein may be split into two fragments at any C, T, A, or S. In some embodiments, a Cas9 may be intein split at residues 203-204, 280-292, 292-364, 311-325, 417-438, 445-483, 468-469, 481-502, 513-520, 522-530, 565-637, 696-707, 713-714, 795-804, 803-810, 878-887, and 1153-1154. In some embodiments, protein is divided into two fragments at SpCas9 T310, T313, A456, S469, or C574. In some embodiments, split Cas9 fragments across different split pairs yield combinations that provided the complete polypeptide sequence activate gene expression even when fragments are partially redundant. In some embodiments, a functional Cas9 protein may be reconstituted from two inactive split-Cas9 peptides in the presence of gR′rA by using a split-intein protein splicing strategy. In some embodiment, the split Cas9 fragments are fused to either a N-terminal intein fragment or a C-terminal intein fragment, which can associate with each other and catalytically splice the two split Cas9 fragments into a functional reconstituted Cas9 protein. In some embodiments, a split-Cas9 can be packaged into self-complementary AAV. In some embodiments, a split-Cas9 comprises a 2.5 kb and a 2.2 kb fragment of S. pyogenes Cas9 coding sequences.

In some embodiments, a split-Cas9 architecture reduces the length and/or size of the coding sequences of a viral vector, e.g., AAV.

A target cell can be transiently or non-transiently transfected with one or more vectors described herein. A cell can be transfected as it naturally occurs in a subject. A cell can be taken or derived from a subject and transfected. A cell can be derived from cells taken from a subject, such as a cell line. In some embodiments, a cell transfected with one or more vectors described herein can be used to establish a new cell line comprising one or more vector-derived sequences. In some embodiments, a cell transiently transfected with the compositions of the disclosure (such as by transient transfection of one or more vectors, or transfection with RNA), and modified through the activity of a prime editor, can be used to establish a new cell line comprising cells containing the modification but lacking any other exogenous sequence. Any suitable vector compatible with the host cell can be used with the methods of the disclosure. Non-limiting examples of vectors include pXTi, pSC5, pSVK3, pBPV, pMSG, and pSVLSV40.

In some embodiments, a prime editor protein can be provided to cells as a polypeptide. In some embodiments, the prime editor protein is fused to a polypeptide domain that increases solubility of the protein. In some embodiments, the prime editor protein is formulated to improve solubility of the protein.

In some embodiment, a prime editor polypeptide is fused to a polypeptide permeant domain to promote uptake by the cell. In some embodiments, the permeant domain is a including peptide, a peptidomimetic, or a non-peptide carrier. For example, a permeant peptide may be derived from the third alpha helix of Drosophila melanogaster transcription factor Antennapaedia, referred to as penetratin, which comprises the amino acid sequence RQ1K1WFQNRRMKWKK (SEQ ID NO: 673). As another example, the permeant peptide can comprise the HIV-1 tat basic region amino acid sequence, which may include, for example, amino acids 49-57 of naturally-occurring tat protein. Other permeant domains can include poly-arginine motifs, for example, the region of amino acids 34-56 of HIV-1 rev protein, nona-arginine, and octa-arginine. The nona-arginine (R9) sequence can be used. The site at which the fusion can be made may be selected in order to optimize the biological activity, secretion or binding characteristics of the polypeptide.

In some embodiments, a prime editor polypeptide is produced in vitro or by host cells, and it may be further processed by unfolding, e.g., heat denaturation, DTT reduction, etc. and may be further refolded. In some embodiments, a prime editor polypeptide is prepared by in vitro synthesis. Various commercial synthetic apparatuses can be used. By using synthesizers, naturally occurring amino acids can be substituted with unnatural amino acids. In some embodiments, a prime editor polypeptide is isolated and purified in accordance with recombinant synthesis methods, for example, by expression in a host cell and the lysate purified using, HPLC, exclusion chromatography, gel electrophoresis, affinity chromatography, or other purification technique.

In some embodiments, a prime editing composition, for example, prime editor polypeptide components and PEgRNA/ngRNA are introduced to a target cell by nanoparticles. In some embodiments, the prime editor polypeptide components and the PEgRNA and/or ngRNA form a complex in the nanoparticle. Any suitable nanoparticle design can be used to deliver genome editing system components or nucleic acids encoding such components. In some embodiments, the nanoparticle is inorganic. In some embodiments, the nanoparticle is organic. In some embodiments, a prime editing composition is delivered to a target cell, e.g., a primary cell, iPSC, fibroblast, hair cell, inner hair cell, outer hair cell, Müller cell, or photoreceptor cell, in an organic nanoparticle, e.g., a lipid nanoparticle (LNP) or polymer nanoparticle.

In some embodiments, LNPs are formulated from cationic, anionic, neutral lipids, or combinations thereof. In some embodiments, neutral lipids, such as the fusogenic phospholipid DOPE or the membrane component cholesterol, are included to enhance transfection activity and nanoparticle stability. In some embodiments, LNPs are formulated with hydrophobic lipids, hydrophilic lipids, or combinations thereof. Lipids may be formulated in a wide range of molar ratios to produce an LNP. Any lipid or combination of lipids that are known in the art can be used to produce an LNP. Exemplary lipids used to produce LNPs are provided in Table 8 below.

In some embodiments, components of a prime editing composition form a complex prior to delivery to a target cell.]For example, a prime editor fusion protein, a PEgRNA, and/or a ngRNA can form a complex prior to delivery to the target cell. In some embodiments, a prime editing polypeptide (e.g., a prime editor fusion protein) and a guide polynucleotide (e.g., a PEgRNA or ngRNA) form a ribonucleoprotein (RNP) for delivery to a target cell. In some embodiments, the RNP comprises a prime editor fusion protein in complex with a PEgRNA. RNPs may be delivered to cells using known methods, such as electroporation, nucleofection, or cationic lipid-mediated methods, or any other approaches known in the art. In some embodiments, delivery of a prime editing composition or complex to the target cell does not require the delivery of foreign DNA into the cell. In some embodiments, the RNP comprising the prime editing complex is degraded over time in the target cell. Exemplary lipids for use in nanoparticle formulations and/or gene transfer are shown in Table 8 below.

TABLE 8
Exemplary lipids for nanoparticle formulation or gene transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3-phosphatidylethanolamine | DOPE | Helper |
| Cholesterol | | Helper |
| N41-(2,3-Dioleyloxy)prophyliN,N,N-trimethylammonium | DOTMA | Cationic |
| chloride | | |
| 1,2-Dioleoyloxy-3-trimethylammonium-propane | DOGS | Cationic |
| Dioctadecylamidoglycylspermine | | |
| N-(3-Aminopropy1)-N,N-dimethy1-2,3-bis(dodecyloxy)-1- | GAP-DLRIE | Cationic |
| propanaminium bromide | | |
| Cetyltrimethylammonium bromide | CTAB | Cationic |
| 6-Lauroxyhexyl omithinate | LHON | Cationic |
| 1-(2,3-Dioleoyloxypropy1)-2,4,6-trimethylpyridinium | 2Oc | Cationic |
| 2,3-Dioleyloxy-N-P(spenninecarboxamido-ethy1J- | DOSPA | Cationic |
| N,Ndimethyl- | | |
| 1-propanatninium trifluoroacetate | | |
| 1,2-Dioley1-3-trimethylamtnonium-propane | DOPA | Cationic |
| N-(2-Hydroxyethyl)-N,N-dimethy1-2,3-bis(tetradecyloxy)-1- | MDRIE | Cationic |
| propanaminium bromide | | |
| Dimyristooxypropyl dimethyl hydroxyethyl ammonium | DMRI | Cationic |
| bromide | | |
| 3β-[N-(N′,N′-Dimethylaminoethane)-carbamoyl]cholesterol | DC-Chol | Cationic |
| Bis-guanidium-tren-cholesterol | BGTC | Cationic |
| 1,3-Diodeoxy-2-(6-carboxy-spermy1)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)]- | CLIP-1 | Cationic |
| dimethylammonium chloride | | |
| rac-[2(2,3-Dihexadecyloxypropyloxymethyloxy) | CLIP-6 | Cationic |
| ethyl]trimethylammoniun bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylpho sphocholine | DSEPC | Cationic |
| N-Palmitoyl D-erythro-sphingosyl carbamoyl-spenmine | CCS | Cationic |
| N-t-Butyl-N0-tetradecyl-3-tetradecylaminopropionamidine | diC14- | Cationic |
| | amidine | |
| Octadecenolyoxy[ethyl-2-heptadecenyl-3 hydroxyethyl] | DOTIM | Cationic |
| imidazolinium chloride | | |
| N1-Cholesteryloxycarbonyl-3,7-diazanonane-1,9-diamine | CDAN | Cationic |
| 2-(3-Bis(3-amino-propy1)-amino]propylamino)- | RPR209120 | Cationic |
| Nditetradecylcarbamoylme-ethyl-acetamide | | |
| 1,2-dilinoleyloxy-3-dimethylaminopropane | DLinDMA | Cationic |
| 2,2-dilinoley1-4-dimethylaminoethyl-[1,3]-dioxolane | DLin-KC2- | Cationic |
| | DMA | |
| dilinoleyl-methyl-4-dimethylaminobutyrate | DLin-MC3- | Cationic |
| | DMA | |

Exemplary polymners for use in natnoparticie formulations and/or gene transfer are shown in Table 9 below.

TABLE 9
Exemplary lipids for nanoparticle formulation or gene transfer

| Polymer | Abbreviation |
|---|---|
| Poly(ethylene)glycol | PEG |
| Polyethylenimine | PEI |
| Dithiobis (succinimidylpropionate) | DSP |
| Dimethyl-3,3′-dithiobispropionimidate | DTBP |
| Poly(ethylene imine)biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amidoethylenimine) | SS_PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-dodacylated chitosam | |
| Histone | |
| Collagen | |
| Dextran-spermine | D-SPM |

Exemplary delivery methods for poly nucleotides encoding prime editing composition components are shown in Table 10 below.

TABLE 10
Exemplary polynucleotide delivery methods

| | | Delivery | | | |
|---|---|---|---|---|---|
| | | into Non- | | | Type of |
| | | Dividing | Duration of | Genome | Molecule |
| Delivery | Vector/Mode | Cells | Expression | Integration | Delivered |
| Physical | (e.g., | YES | Transient | NO | Nucleic |
| | electroporation, | | | | Acids and |
| | particle gun, | | | | Proteins |
| | Calcium | | | | |
| | phosphate | | | | |
| | transfection) | | | | |
| Viral | Retrovirus | NO | Stable | YES | RNA |
| | Lentivirus | YES | Stable | YES/NO | RNA |
| | | | | with | |
| | | | | modification | |
| | Adenovirus | YES | Transient | NO | DNA |
| | Adeno- | YES | Stable | NO | DNA |
| | Associated Virus | | | | |
| | (AAV) | | | | |
| | Vaccinia Virus | YES | Very | NO | DNA |
| | | | Transient | | |
| | Herpes Simplex | YES | Stable | NO | DNA |
| | Virus | | | | |
| Non-Viral | Cationic | YES | Transient | Depends on | Nucleic |
| | | | | what is | acids and |
| | | | | delivered | Proteins |
| | Polymeric | YES | Transient | NO | Nucleic |
| | Nanoparticles | | | | Acids |
| Biological | Attenuated | YES | Transient | NO | Nucleic |
| | Bacteria | | | | Acids |
| Non-Viral | Engineered | YES | Transient | NO | Nucleic |
| Delivery | Bacteriophages | | | | Acids |
| Vehicles | Mammalian | YES | Transient | NO | Nucleic |
| | Virus-like | | | | Acids |
| | Particles | | | | |
| | Biological | YES | Transient | NO | Nucleic |
| | liposomes: | | | | Acids |
| | Erythrocyte | | | | |
| | Ghosts and | | | | |
| | Exosomes | | | | |

The prime editing compositions of the disclosure, whether introduced as polYnucieotides or polypeptides, can be provided to the cells for about 30 minutes to about 24 hours, e.g., 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which can be repeated with a frequency of about every day to about every 4 days, e.g., every 1.5 days, every 2 days, every 3 days, or any other frequency from about every day to about every four days. The compositions may be provided to the subject cells one or more times, e.g., one time, twice, three times, or more than three times, and the cells allowed to incubate with the agent(s) for some amount of time following each contacting event e.g., 16-24 hours. In cases in which two or more different prime editing system components, e.g., two different polynucleotide constructs are provided to the cell (e.g., different components of the same prime editing system, or two different guide nucleic acids that are complementary to different sequences within the same or different target genes), the compositions may be delivered simultaneously (e.g., as two polypeptides and/or nucleic acids). Alternatively, they may be provided sequentially, e.g., one composition being provided first, followed by a second composition.

The prime editing compositions and pharmaceutical compositions of the disclosure, wlhether introduced as polynucleotides or polypeptides, can be administered to subjects in need thereof for about 30 minutes to about 24 hours, e.g., 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which can be repeated with a frequency of about every day to about every 4 days, e.g., every 1.5 days, every 2 days, every 3 days, or any other frequency from about every day to about every four days. The compositions may be provided to the subject one or more times, e.g., one time, twice, three times, or more than three times. In cases in which two or more different prime editing system components, e.g., two different polynucleotide constructs are administered to the subject (e.g., different components of the same prime editing system, or two different guide nucleic acids that are complementary to different sequences within the same or different target genes), the compositions may be administered simultaneously (e.g., as two polypeptides and/or nucleic acids). Alternatively, they may be provided sequentially, e.g., one composition being provided first, followed by a second composition.

### EXAMPLES

The following examples are provided for illustrative purposes only and are not intended to limit the scope of the claims provided herein.

### Example 1—General Methods

PEgRNA assembly: PEgRNA libraries may be assembled by one of three methods: in the first method, pooled synthesized DNA oligos encoding the PEgRNA and flanking U6 expression plasmid homology regions may be cloned into U6 expression plasmids via Gibson cloning and sequencing of bacterial colonies via Sanger or Next-generation sequencing. In the second method, double-stranded linear DNA fragments encoding PEgRNA and homology sequences as above may be individually Gibson-cloned into U6 expression plasmids. In the third method, for each PEgRNA, separate oligos encoding a protospacer, a gRNA scaffold, and PEgRNA extension (PBS and RTT) may be ligated, and then cloned into a U6 expression plasmid as described in Anzalone et al., Nature. 2019 Dec;576(7785):149-157. Bacterial colonies carrying sequence-verified plasmids may be propagated in LB or TB. Plasmid DNA may be purified by minipreps for mammalian transfection.

PEgRNA may also be chemically synthesized. Such chemically synthesized PEgRNAs may be modified at the 5′ end and the 3′ end: the three 5′ most nucleotides may be modified to phosphorothioated 2′-O-methyl nucleotides. The three consecutive nucleotides that precedes the 3′ most nucleotide (i.e. three consecutive nucleotides immediately 5′ of the last nucleotide at the 3′ end) may also modified to phosphorothioated 2′-O-methyl nucleotides.

HEK cell culture and transfection: HEK293T cells may be propagated in DMEM with 10% FBS. Prior to transfection, cells may be seeded in 96-well plates and then transfected with Lipofectamine 2000 or MessengerMax according to the manufacturer's directions with DNA or mRNA encoding a prime editor and PEgRNA (and ngRNA for PE3 experiments). Three days after transfection, gDNA may be harvested in lysis buffer for high throughput sequencing and may be sequenced using Miseq.

Lentiviral production and cell line generation—Generation of cells lines carrying a CLRN1 c.144T->G mutation (N48K) cassette: Lentiviral transfer plasmids containing the CLRN1 c.144T->G mutation (N48K) with flanking sequences from the CLRN1 gene on each side, and an IRES-Puromycin selection cassette, may be cloned behind an EFla short promoter. HEK293T cells may be transiently transfected with the transfer plasmids and packaging plasmids containing VSV glycoprotein and lentiviral gag/pol coding sequences. After transfection, lentiviral particles may be harvested from the cell media and concentrated. HEK293T cells may be transduced using serial dilutions of the lentiviral particles described above. Cells generated at a dilution of MOI <1, as determined by survival following puromycin, are selected for expansion. A resulting HEK293T cell line carrying the c.144T->G mutation may be used to screen PEgRNAs.

Installation of N48K mutation by prime editing: Generation of cell lines carrying a CLRN1 c.144T->G mutation (N48K) in the endogenous CLRN1 gene: PEgRNAs for NGG PAM recognition may be designed to incorporate a CLRN1 c.144T->G mutation in the wild type endogenous CLRN1 gene in HEK293T cells by prime editing as a proxy to examine editing efficiency.

A wild type HEK293T cell line may be expanded and transiently transfected with a nucleic acid encoding a prime editor and an N48K mutation installation PEgRNA in arrayed 96-well plates for assessment of editing by high-throughput sequencing. Prior to transfection, cells may be seeded in 96-well plates and then transfected with Lipofectamine 2000 or MessengerMax according to the manufacturer's directions with DNA or mRNA and PEgRNA. Three days after transfection, gDNA may be harvested in lysis buffer for high throughput sequencing, which may be sequenced using Miseq.

Usher Syndrome type 3 mutation correction with PE2 system: A HEK293T cell line carrying the N48K mutation, such as one made by a method described above, may be expanded and transiently transfected with a prime editor and PEgRNA in arrayed 96-well plates for assessment of editing by high-throughput sequencing.

Usher Syndrome type 3 mutation correction with PE3 system: a nick guide RNA (“ngRNA”) that causes a nick on the opposite strand compared to the PEgRNA (i.e., on the non-edit strand) may be included in the transfection mixture described above. Addition of a ngRNA may improve efficiency and/or fidelity of prime editing.

### Example 2—Installation of N48K Mutation by Prime Editing

PEgRNAs for NGG PAM recognition were designed to incorporate a CLRN1 c.144T->G mutation in the wild type endogenous CLRN1 gene in HEK293T cells by prime editing.

Briefly, a wild type HEK293T cell line was expanded and transiently transfected with a mRNA encoding a prime editor fusion protein and a N48K mutation installation PEgRNA in arrayed 96-well plates for assessment of editing by high-throughput sequencing. Prior to transfection, cells were seeded in 96-well plates and then transfected with MessengerMax according to the manufacturer's directions. Three days after transfection, gDNA was harvested in lysis buffer for high throughput sequencing and sequenced using Miseq. The clones containing the N48K mutation were banked and registered to be used in future correction experiments.

### Example 3—Screening of PEgRNA for Editing of a Mutation Associated with Usher Syndrome type 3

The PEgRNAs used in this experiment were chemically synthesized by Integrated DNA Technologies (IDT). Chemically synthesized PEgRNAs were modified at the 5′ end and the 3′ end: the three 5′ most nucleotides were modified to phosphorothioated 2′-O-methyl nucleotides. The three consecutive nucleotides that precedes the 3′ most nucleotide (i.e. three consecutive nucleotides immediately 5′ of the last nucleotide at the 3′ end) were also modified to phosphorothioated 2′-O-methyl nucleotides. The PEgRNA tested here contained edit templates/RTTs that ranged in length from 13 to 26 nucleotides and primer binding sites (PBSs) from 8 to 16 nucleotides in length. All PEgRNA tested were designed to correct a CLRN1 c. 144T->G mutation. All but 3 PEgRNAs encoded wild-type CLRN1 sequence; the remainder encoded synonymous PAM silencing mutations.

An HEK293T cell line carrying the N48K mutation generated according to Example 2 was expanded and transiently transfected with mRNA encoding a Prime Editor fusion protein and PEgRNA in arrayed 96-well plates for assessment of editing by high-throughput sequencing.

The results are shown in Table 11 below. Successful correction by prime editing at the CLRN1 c. 144T->G mutation site in HEK293T cells was observed at all RTT and PBS lengths tested. In fact, all PEgRNA tested except for 1 yielded precise correction levels well above those observed in the non-transfection control samples.

TABLE 11
Prime Editing at a mutation site in HEK293T cells with a PEgRNA (PE2 system)

| PEgRNA¹ | Spacer | RTT² | | PBS | | | |
|---|---|---|---|---|---|---|---|
| Sequence | Sequence | Sequence | RTT | Sequence | PBS | % | % |
| Number | Number | Number | Length | Number | Length | Edit³ | Indel³ |
| 352 | 4 | 28 | 13 | 10 | 8 | 18.04% | 0.74% |
| 359 | 4 | 39 | 15 | 10 | 8 | 13.62% | 0.43% |
| 387 | 4 | 54 | 18 | 10 | 8 | 6.09% | 0.20% |
| 448 | 4 | 71 | 22 | 10 | 8 | 3.49% | 0.12% |
| 470 | 4 | 79 | 24 | 10 | 8 | 6.16% | 0.17% |
| 487 | 4 | 87 | 26 | 10 | 8 | 6.84% | 0.18% |
| 360 | 4 | 28 | 13 | 12 | 10 | 41.68% | 2.04% |
| 376 | 4 | 39 | 15 | 12 | 10 | 33.01% | 1.03% |
| 421 | 4 | 54 | 18 | 12 | 10 | 10.71% | 0.22% |
| 471 | 4 | 71 | 22 | 12 | 10 | 8.58% | 0.23% |
| 488 | 4 | 79 | 24 | 12 | 10 | 5.51% | 0.10% |
| 496 | 4 | 87 | 26 | 12 | 10 | 9.43% | 0.18% |
| 377 | 4 | 28 | 13 | 14 | 12 | 33.51% | 1.19% |
| 402 | 4 | 39 | 15 | 14 | 12 | 28.62% | 0.60% |
| 353 | 4 | 28 | 13 | 11 | 9 | 24.37% | 0.68% |
| 449 | 4 | 54 | 18 | 14 | 12 | 31.50% | 0.84% |
| 366 | 4 | 28 | 13 | 13 | 11 | 50.51% | 3.15% |
| 489 | 4 | 71 | 22 | 14 | 12 | 24.28% | 0.68% |
| 388 | 4 | 28 | 13 | 15 | 13 | 49.67% | 2.64% |
| 497 | 4 | 79 | 24 | 14 | 12 | 19.67% | 0.32% |
| 422 | 4 | 28 | 13 | 17 | 15 | 31.36% | 0.81% |
| 502 | 4 | 87 | 26 | 14 | 12 | 15.28% | 0.17% |
| 367 | 4 | 39 | 15 | 11 | 9 | 12.58% | 0.20% |
| 389 | 4 | 39 | 15 | 13 | 11 | 28.06% | 0.65% |
| 423 | 4 | 39 | 15 | 15 | 13 | 33.24% | 1.09% |
| 403 | 4 | 28 | 13 | 16 | 14 | 31.10% | 0.82% |
| 450 | 4 | 39 | 15 | 17 | 15 | 34.79% | 0.73% |
| 436 | 4 | 39 | 15 | 16 | 14 | 25.33% | 0.51% |
| 378 | 4 | 48 | 17 | 10 | 8 | 8.54% | 0.18% |
| 390 | 4 | 48 | 17 | 11 | 9 | 20.77% | 0.56% |
| 404 | 4 | 48 | 17 | 12 | 10 | 32.70% | 1.09% |
| 424 | 4 | 48 | 17 | 13 | 11 | 33.16% | 0.79% |
| 437 | 4 | 48 | 17 | 14 | 12 | 16.53% | 0.23% |
| 451 | 4 | 48 | 17 | 15 | 13 | 14.55% | 0.22% |
| 460 | 4 | 48 | 17 | 16 | 14 | 2.67% | 0.06% |
| 472 | 4 | 48 | 17 | 17 | 15 | 0.19% | 0.01% |
| 480 | 4 | 48 | 17 | 18 | 16 | 31.20% | 0.65% |
| 473 | 4 | 54 | 18 | 16 | 14 | 14.02% | 0.32% |
| 405 | 4 | 54 | 18 | 11 | 9 | 13.96% | 0.49% |
| 498 | 4 | 71 | 22 | 16 | 14 | 20.88% | 0.46% |
| 438 | 4 | 54 | 18 | 13 | 11 | 20.19% | 0.37% |
| 503 | 4 | 79 | 24 | 16 | 14 | 15.63% | 0.19% |
| 461 | 4 | 54 | 18 | 15 | 13 | 31.19% | 0.72% |
| 506 | 4 | 87 | 26 | 16 | 14 | 23.02% | 0.29% |
| 481 | 4 | 54 | 18 | 17 | 15 | 8.90% | 0.12% |
| 425 | 4 | 63 | 20 | 10 | 8 | 2.06% | 0.05% |
| 439 | 4 | 63 | 20 | 11 | 9 | 3.33% | 0.06% |
| 452 | 4 | 63 | 20 | 12 | 10 | 15.78% | 0.34% |
| 462 | 4 | 63 | 20 | 13 | 11 | 24.38% | 0.68% |
| 474 | 4 | 63 | 20 | 14 | 12 | 18.01% | 0.23% |
| 482 | 4 | 63 | 20 | 15 | 13 | 18.82% | 0.28% |
| 490 | 4 | 63 | 20 | 16 | 14 | 17.26% | 0.33% |
| 492 | 4 | 63 | 20 | 17 | 15 | 25.64% | 0.51% |
| 499 | 4 | 63 | 20 | 18 | 16 | 21.83% | 0.36% |
| 463 | 4 | 71 | 22 | 11 | 9 | 5.00% | 0.16% |
| 483 | 4 | 71 | 22 | 13 | 11 | 10.97% | 0.21% |
| 440 | 4 | 28 | 13 | 18 | 16 | 38.16% | 1.42% |
| 493 | 4 | 71 | 22 | 15 | 13 | 16.59% | 0.27% |
| 464 | 4 | 39 | 15 | 18 | 16 | 34.76% | 0.77% |
| 500 | 4 | 71 | 22 | 17 | 15 | 20.89% | 0.44% |
| 491 | 4 | 54 | 18 | 18 | 16 | 20.48% | 0.27% |
| 504 | 4 | 71 | 22 | 18 | 16 | 15.85% | 0.17% |
| 484 | 4 | 79 | 24 | 11 | 9 | 1.16% | 0.03% |
| 507 | 4 | 79 | 24 | 18 | 16 | 3.15% | 0.03% |
| 494 | 4 | 79 | 24 | 13 | 11 | 4.04% | 0.03% |
| 508 | 4 | 87 | 26 | 18 | 16 | 4.27% | 0.03% |
| 501 | 4 | 79 | 24 | 15 | 13 | 2.66% | 0.03% |
| 505 | 4 | 79 | 24 | 17 | 15 | 3.45% | 0.04% |
| 495 | 4 | 87 | 26 | 11 | 9 | 1.09% | 0.02% |
| 406 | 4 | 40* | 15 | 14 | 12 | 57.42% | 0.39% |
| 407 | 4 | 38* | 15 | 14 | 12 | 24.89% | 0.19% |
| 408 | 4 | 41* | 15 | 14 | 12 | 16.24% | 0.15% |

¹The indicated sequence contains, from 5′ to 3′, the indicated Spacer sequence, a gRNA core according to SEQ ID NO: 665, the indicated RTT sequence, the indicated PBS sequence, a Linker (AACATTGA; Sequence Number 671) and a 3′ hairpin motif (CGCGTCTCTACGTGGGGGCGCG; SEQ ID NO: 672). The PEgRNA used experimentally further contained 3′ mN*mN*mN*N and 5′mN*mN*mN* modifications, where m indicates that the nucleotide contains a 2′-O—Me modification and a * indicates a phosphorothioate bond.
²*= RTT encodes a synonymous PAM silencing edit; Sequence Number 40 encodes an AGG-to-ACG PAM silencing edit, Sequence Number 38 encodes an AGG-to-ATG PAM silencing edit, and Sequence Number 41 encodes an AGG-to-AAG PAM silencing edit.
³Six non-transfection control replicates were performed which yielded an average percent correction of 0.10% with a standard deviation of 0.01% and an average percent indel of 0.01% with a standard deviation of 0.01%.

### Example 4—Screening of PEgRNA and ngRNA for Editing of a Mutation associated with Usher Syndrome type 3

The PEgRNAs used in this experiment were chemically synthesized by Integrated DNA Technologies (IDT). Chemically synthesized PEgRNAs were modified at the 5′ end and the 3′ end: the three 5′ most nucleotides were modified to phosphorothioated 2′-O-methyl nucleotides. The three consecutive nucleotides that precedes the 3′ most nucleotide (i.e. three consecutive nucleotides immediately 5′ of the last nucleotide at the 3′ end) were also modified to phosphorothioated 2′-O-methyl nucleotides. The PEgRNA tested here contained edit templates/RTTs that ranged in length from 12 to 18 nucleotides and primer binding sites (PBSs) from 9 to 15 nucleotides in length. The PEgRNA tested encode either wild-type CLRN1 sequence or contain an AGG-to-AGC nonsynonymous [A49G] PAM silencing edit. All PEgRNA tested were designed to correct a CLRN1 c.144T->G mutation.

An HEK293T cell line carrying the N48K mutation generated according to Example 2 was expanded and transiently transfected with mRNA encoding a Prime Editor fusion protein and PEgRNA and ngRNA in arrayed 96-well plates for assessment of editing by high-throughput sequencing.

The results are shown in Tables 12a to 12e. Details on the PEgRNA tested can be found in Table 13.

TABLE 12a
Prime Editing at a mutation site in HEK293T
cells with a PEgRNA and ngRNA (PE3 system)

| PEgRNA^{1,2} | ngRNA spacer³ Sequence Number: | | | | | |
|---|---|---|---|---|---|---|
| Sequence | PE2 | | 182 | | 176 | |
| Number: | % Edit | % indel | % Edit | % indel | % Edit | % indel |
| 368 | 24.92% | 5.36% | 12.15% | 22.54% | 52.24% | 10.56% |
| 379 | 25.61% | 5.87% | 8.93% | 33.62% | 28.89% | 23.36% |
| 39 | 26.52% | 4.26% | 9.51% | 25.12% | 1.13% | 0.09% |
| 409 | 26.58% | 4.05% | 9.29% | 26.69% | 14.51% | 30.82% |
| 355* | 0.22% | 0.01% | 27.75% | 15.45% | 15.24% | 8.26% |
| 362* | 7.15% | 4.13% | 26.10% | 15.09% | 18.68% | 15.99% |
| 369* | 0.17% | 0.02% | 21.23% | 13.19% | 17.20% | 8.70% |
| 380* | 7.83% | 4.25% | 22.32% | 14.46% | 23.23% | 17.42% |
| 392* | 6.52% | 3.09% | 15.68% | 13.24% | 13.52% | 8.97% |
| 410* | 5.95% | 2.92% | 18.09% | 14.46% | 17.29% | 14.69% |
| 356* | 54.21% | 0.88% | 67.81% | 8.01% | 73.84% | 2.20% |
| 363* | 58.62% | 1.14% | 68.58% | 7.99% | 56.51% | 5.98% |
| 370* | 75.00% | 1.65% | 74.96% | 3.55% | 65.75% | 6.45% |
| 381* | 81.46% | 1.89% | 77.71% | 6.56% | 75.86% | 5.64% |
| 393* | 81.24% | 1.26% | 77.11% | 7.73% | 80.97% | 2.39% |
| 411* | 79.35% | 1.37% | 79.51% | 1.37% | 77.34% | 5.09% |
| 426* | 80.47% | 1.21% | 81.08% | 1.40% | 81.70% | 3.46% |
| 371* | 78.84% | 1.01% | 74.55% | 7.40% | 71.36% | 6.71% |
| 382* | 78.68% | 1.19% | 80.79% | 2.25% | 72.67% | 6.96% |
| 394* | 81.69% | 0.95% | 78.03% | 1.53% | 80.46% | 0.99% |
| 412* | 84.72% | 0.85% | 80.03% | 1.65% | 77.29% | 5.95% |
| 427* | 84.49% | 0.78% | 72.28% | 6.74% | 75.29% | 5.66% |
| 441* | 81.99% | 0.94% | 78.43% | 1.46% | 67.62% | 5.65% |
| 453* | 81.58% | 0.66% | 74.88% | 0.79% | 56.82% | 5.32% |
| 395* | 70.62% | 0.84% | 4.34% | 3.10% | 65.04% | 7.00% |
| 413* | 78.40% | 1.05% | 59.34% | 4.15% | 69.22% | 6.68% |
| 428* | 78.89% | 1.46% | 77.17% | 2.33% | 67.97% | 8.01% |
| 442* | 83.07% | 1.19% | 81.52% | 1.93% | 79.31% | 1.48% |
| 454* | 83.37% | 0.99% | 81.30% | 2.13% | 80.76% | 1.20% |
| 465* | 82.78% | 0.85% | 83.13% | 2.90% | 81.51% | 0.98% |
| 475* | 82.04% | 0.92% | 75.90% | 8.62% | 82.44% | 1.19% |
| 414* | 76.17% | 0.81% | 62.65% | 11.28% | 76.73% | 0.94% |
| 429* | 77.00% | 1.00% | 61.67% | 10.04% | 62.04% | 7.49% |
| 443* | 76.43% | 0.59% | 75.74% | 1.04% | 55.64% | 6.71% |
| 455* | 76.67% | 0.87% | 58.37% | 8.98% | 64.19% | 7.14% |
| 466* | 78.98% | 0.85% | 47.00% | 6.93% | 62.61% | 5.64% |
| 476* | 76.04% | 0.73% | 62.73% | 9.11% | 76.83% | 1.04% |
| 485* | 86.02% | 0.98% | 74.58% | 9.65% | 70.62% | 5.23% |
| 354 | 28.74% | 11.05% | 25.68% | 21.73% | 12.38% | 29.26% |
| 361 | 31.91% | 8.31% | 9.51% | 47.48% | 14.73% | 29.43% |

¹The indicated PEgRNA sequence contains, from 5′ to 3′, a Spacer, a gRNA core according to SEQ ID NO: 665, an RTT, a PBS, a Linker (AACATTGA; Sequence Number 671) and a 3′ hairpin motif (CGCGTCTCTACGTGGGGGCGCG; SEQ ID NO: 672). The PEgRNA used experimentally further contained 3′ mN*mN*mN*N and 5′mN*mN*mN* modifications, where m indicates that the nucleotide contains a 2′-O—Me modification and a * indicates a phosphorothioate bond. Please see table 13 below for information on the Spacer, RTT, and PBS sequences used in the indicated PEgRNAs.
²A * indicates that the indicated PEgRNA encodes an AGG-to-AGC nonsynonymous [A49G] PAM silencing edit; lack of a * indicates the PEgRNA encodes wild-type CLRNI sequence.
³The ngRNA used experimentally contained, from 5′ to 3′, the indicated Spacer sequence, a gRNA core according to SEQ ID NO: 665, and a 3′ UUUU sequence. The ngRNA further contained 3′ mN*mN*mN*N and 5′mN*mN*mN* modifications, where m indicates that the nucleotide contains a 2′-O—Me modification and a * indicates a phosphorothioate bond. PE2 indicates that no ngRNA was used.

TABLE 12b
Prime Editing at a mutation site in HEK293T
cells with a PEgRNA and ngRNA (PE3 system)

| PERNA^{1,2} | ngRNA spacer³ Sequence Number: | | | | | |
|---|---|---|---|---|---|---|
| Sequence | 171 | | 180 | | 173 | |
| Number: | % Edit | % indel | % Edit | % indel | % Edit | % indel |
| 368 | 27.43% | 13.60% | 28.87% | 9.85% | 17.01% | 4.18% |
| 379 | 26.77% | 14.44% | 28.32% | 12.69% | 26.14% | 8.79% |
| 391 | 27.64% | 6.79% | 31.08% | 10.48% | 29.40% | 6.19% |
| 409 | 24.41% | 12.38% | 25.59% | 9.34% | 28.89% | 5.95% |
| 355* | 16.69% | 18.17% | 14.11% | 11.27% | 16.50% | 7.36% |
| 362* | 15.07% | 18.68% | 12.17% | 10.43% | 2.90% | 2.06% |
| 369* | 10.10% | 5.95% | 15.99% | 12.04% | 14.44% | 6.92% |
| 380* | 14.27% | 14.81% | 7.48% | 7.26% | 13.08% | 6.23% |
| 392* | 10.58% | 13.11% | 9.42% | 9.23% | 1.56% | 1.16% |
| 410* | 12.33% | 6.47% | 9.42% | 8.44% | 1.84% | 0.97% |
| 356* | 65.49% | 5.41% | 59.60% | 3.53% | 34.70% | 1.09% |
| 363* | 64.42% | 6.86% | 61.16% | 3.96% | 37.80% | 1.07% |
| 370* | 72.52% | 1.67% | 75.52% | 1.74% | 52.41% | 1.79% |
| 381* | 77.56% | 6.86% | 81.83% | 1.81% | 80.67% | 1.41% |
| 393* | 77.84% | 6.21% | 77.28% | 5.09% | 80.80% | 1.36% |
| 411* | 75.07% | 6.32% | 79.60% | 1.15% | 80.17% | 1.50% |
| 426* | 65.65% | 3.66% | 74.68% | 1.45% | 80.08% | 1.26% |
| 371* | 71.79% | 5.22% | 68.56% | 3.74% | 80.49% | 1.09% |
| 382* | 73.42% | 6.79% | 78.04% | 1.15% | 41.75% | 1.41% |
| 394* | 74.71% | 5.51% | 71.50% | 0.79% | 28.58% | 0.67% |
| 412* | 73.58% | 6.70% | 71.44% | 3.45% | 50.86% | 1.46% |
| 427* | 72.69% | 5.32% | 75.37% | 3.47% | 47.30% | 1.00% |
| 441* | 71.45% | 4.88% | 66.49% | 2.45% | 44.26% | 1.04% |
| 453* | 68.77% | 3.92% | 75.79% | 0.92% | 75.63% | 0.93% |
| 395* | 61.86% | 4.93% | 73.83% | 1.18% | 75.27% | 0.80% |
| 413* | 79.86% | 2.21% | 68.90% | 2.43% | 48.03% | 1.24% |
| 428* | 75.53% | 6.14% | 72.18% | 3.45% | 57.08% | 1.13% |
| 442* | 79.72% | 5.28% | 78.16% | 1.07% | 82.63% | 0.93% |
| 454* | 81.99% | 0.96% | 78.24% | 3.59% | 82.54% | 1.05% |
| 465* | 73.71% | 5.69% | 78.18% | 0.72% | 82.95% | 0.93% |
| 475* | 79.51% | 4.61% | 82.96% | 0.85% | 56.59% | 0.99% |
| 414* | 65.28% | 6.03% | 63.89% | 3.66% | 27.61% | 0.63% |
| 429* | 68.76% | 5.12% | 68.47% | 3.79% | 34.70% | 0.94% |
| 443* | 63.35% | 4.75% | 64.67% | 3.18% | 32.91% | 1.03% |
| 455* | 75.57% | 1.25% | 66.74% | 2.82% | 39.72% | 1.22% |
| 466* | 50.77% | 2.66% | 74.37% | 0.69% | 33.46% | 0.78% |
| 476* | 67.13% | 5.77% | 64.51% | 3.71% | 44.05% | 1.24% |
| 485* | 73.59% | 6.74% | 64.99% | 3.14% | 43.20% | 1.17% |
| 354 | 30.77% | 13.94% | 29.17% | 9.52% | 28.82% | 10.52% |
| 361 | 31.69% | 5.61% | 27.91% | 8.01% | 29.27% | 9.15% |

¹The indicated PEgRNA sequence contains, from 5′ to 3′, a Spacer, a gRNA core according to SEQ ID NO: 665, an RTT, a PBS, a Linker (AACATTGA; Sequence Number 671) and a 3′ hairpin motif (CGCGTCTCTACGTGGGGGCGCG; SEQ ID NO: 672). The PEgRNA used experimentally further contained 3′ mN*mN*mN*N and 5′mN*mN*mN* modifications, where m indicates that the nucleotide contains a 2′-O—Me modification and a * indicates a phosphorothioate bond. Please see table 13 below for information on the Spacer, RTT, and PBS sequences used in the indicated PERNAs.
²A * indicates that the indicated PEgRNA encodes an AGG-to-AGC nonsynonymous [A49G] PAM silencing edit; lack of a * indicates the PEgRNA encodes wild-type CLRNI sequence.
³The ngRNA used experimentally contained, from 5′ to 3′, the indicated Spacer sequence, a gRNA core according to SEQ ID NO: 665, and a 3′ UUUU sequence. The ngRNA further contained 3′ mN*mN*mN*N and 5′mN*mN*mN* modifications, where m indicates that the nucleotide contains a 2′-O—Me modification and a * indicates a phosphorothioate bond. PE2 indicates that no ngRNA was used.

TABLE 12c
Prime Editing at a mutation site in HEK293T
cells with a PEgRNA and ngRNA (PE3 system)

| PEgRNA^{1,2} | ngRNA spacer3 Sequence Number: | | | | | |
|---|---|---|---|---|---|---|
| Sequence | 157 | | 152 | | 161 | |
| Number: | % Edit | % indel | % Edit | % indel | % Edit | % indel |
| 368 | 34.06% | 5.78% | 46.31% | 3.34% | 70.08% | 5.63% |
| 379 | 38.66% | 7.23% | 62.36% | 4.99% | 73.82% | 7.71% |
| 391 | 21.16% | 4.89% | 59.63% | 4.27% | 76.04% | 5.96% |
| 409 | 32.75% | 7.05% | 50.83% | 2.89% | 74.69% | 5.70% |
| 355* | 15.50% | 7.29% | 10.59% | 2.41% | 21.68% | 4.19% |
| 362* | 16.57% | 4.59% | 9.13% | 2.66% | 23.83% | 5.56% |
| 369* | 16.74% | 4.85% | 9.84% | 2.98% | 20.10% | 6.10% |
| 380* | 16.23% | 4.76% | 7.67% | 2.94% | 16.56% | 6.55% |
| 392* | 10.97% | 3.24% | | | 14.84% | 3.44% |
| 410* | 10.60% | 3.42% | 6.73% | 1.96% | 17.27% | 4.18% |
| 356* | 70.80% | 1.08% | 52.82% | 0.68% | 84.87% | 0.94% |
| 363* | 76.20% | 1.03% | 56.74% | 0.79% | 82.20% | 1.15% |
| 370* | 73.32% | 3.68% | 81.66% | 1.83% | 77.98% | 1.33% |
| 381* | 86.82% | 1.35% | 88.23% | 1.39% | 84.79% | 1.05% |
| 393* | 88.31% | 1.31% | 83.53% | 1.04% | 88.26% | 1.04% |
| 411* | 86.56% | 1.24% | 85.91% | 0.85% | 81.38% | 0.78% |
| 426* | 8.11% | 0.34% | 90.51% | 0.92% | 88.47% | 0.79% |
| 371* | 83.04% | 1.96% | 84.73% | 1.06% | 88.15% | 0.90% |
| 382* | 84.87% | 1.98% | 85.07% | 0.72% | 60.43% | 0.46% |
| 394* | 84.95% | 1.95% | 86.45% | 0.98% | 87.77% | 0.87% |
| 412* | 86.68% | 1.49% | | | 88.65% | 0.96% |
| 427* | 82.21% | 1.48% | 89.39% | 0.76% | 89.17% | 0.74% |
| 441* | 86.18% | 1.83% | 90.87% | 0.74% | 82.19% | 0.80% |
| 453* | 81.76% | 1.47% | 84.19% | 0.91% | 80.05% | 0.66% |
| 395* | 82.76% | 1.52% | 56.05% | 0.60% | 61.16% | 0.63% |
| 413* | 86.68% | 1.33% | 85.00% | 0.75% | 69.29% | 0.72% |
| 428* | 67.34% | 1.20% | 88.21% | 1.15% | 69.45% | 0.84% |
| 442* | 86.98% | 1.09% | 90.95% | 0.94% | 80.33% | 0.89% |
| 454* | 86.48% | 1.30% | 91.17% | 0.83% | 79.31% | 1.06% |
| 465* | 87.01% | 1.26% | 88.46% | 0.67% | 76.71% | 0.86% |
| 475* | 88.69% | 1.31% | 91.50% | 0.70% | 80.32% | 0.67% |
| 414* | 76.58% | 1.40% | 81.71% | 0.83% | 76.41% | 0.64% |
| 429* | 78.12% | 1.51% | | | 71.78% | 0.71% |
| 443* | 72.10% | 1.54% | 79.72% | 0.83% | 72.71% | 0.62% |
| 455* | 76.14% | 1.34% | 81.43% | 0.73% | 74.41% | 0.59% |
| 466* | 82.03% | 1.65% | 78.95% | 0.67% | 75.00% | 0.61% |
| 476* | 0.22% | 0.03% | 72.23% | 0.97% | 54.18% | 0.47% |
| 485* | 0.19% | 0.03% | 80.05% | 0.72% | 39.32% | 0.34% |
| 354 | 0.18% | 0.01% | 77.22% | 9.61% | 55.67% | 5.43% |
| 361 | 0.33% | 0.04% | 76.76% | 8.44% | 61.50% | 5.65% |

¹The indicated PEgRNA sequence contains, from 5′ to 3′, a Spacer, a gRNA core according to SEQ ID NO: 665, an RTT, a PBS, a Linker (AACATTGA; Sequence Number 671) and a 3′ hairpin motif (CGCGTCTCTACGTGGGGGCGCG; SEQ ID NO: 672). The PEgRNA used experimentally further contained 3′ mN*mN*mN*N and 5′mN*mN*mN* modifications, where m indicates that the nucleotide contains a 2′-O—Me modification and a * indicates a phosphorothioate bond. Please see table 13 below for information on the Spacer, RTT, and PBS sequences used in the indicated PEgRNAs.
²A * indicates that the indicated PEgRNA encodes an AGG-to-AGC nonsynonymous [A49G] PAM silencing edit; lack of a * indicates the PEgRNA encodes wild-type CLRNI sequence.
³The ngRNA used experimentally contained, from 5′ to 3′, the indicated Spacer sequence, a gRNA core according to SEQ ID NO: 665, and a 3′ UUUU sequence. The ngRNA further contained 3′ mN*mN*mN*N and 5′mN*mN*mN* modifications, where m indicates that the nucleotide contains a 2′-O—Me modification and a * indicates a phosphorothioate bond. PE2 indicates that no ngRNA was used.

TABLE 12d
Prime Editing at a mutation site in HEK293T
cells with a PEgRNA and ngRNA (PE3 system)

| PEgRNA^{1,2} | ngRNA spacer³ Sequence Number: | | | | | |
|---|---|---|---|---|---|---|
| Sequence | 155 | | 147 | | 148 | |
| Number: | % Edit | % indel | % Edit | % indel | % Edit | % indel |
| 368 | 36.02% | 9.15% | 34.02% | 8.49% | 33.07% | 9.16% |
| 379 | 35.78% | 8.38% | 35.45% | 8.54% | 33.69% | 8.65% |
| 391 | 38.41% | 7.37% | 37.91% | 10.25% | 33.79% | 6.97% |
| 409 | 37.32% | 6.45% | 37.48% | 9.93% | 35.13% | 6.08% |
| 355* | | | 28.95% | 8.77% | 18.42% | 7.40% |
| 362* | 17.74% | 5.81% | 28.95% | 8.55% | 18.35% | 6.63% |
| 369* | 15.36% | 4.56% | 25.50% | 7.62% | 19.30% | 7.16% |
| 380* | 20.08% | 6.88% | 29.73% | 8.67% | 20.10% | 7.32% |
| 392* | 10.44% | 3.59% | 23.54% | 6.72% | 16.74% | 5.08% |
| 410* | 13.82% | 4.70% | 25.79% | 6.15% | 16.41% | 5.53% |
| 356* | 71.33% | 1.45% | 78.66% | 2.08% | 76.59% | 1.61% |
| 363* | 69.29% | 1.84% | 69.84% | 1.86% | 74.35% | 1.80% |
| 370* | 0.43% | 0.03% | 58.23% | 1.86% | 70.72% | 1.94% |
| 381* | 79.81% | 2.02% | 71.27% | 1.92% | 78.47% | 1.60% |
| 393* | 85.10% | 1.98% | 80.99% | 1.38% | 77.41% | 1.58% |
| 411* | 79.64% | 2.39% | 79.50% | 1.49% | 76.65% | 1.52% |
| 426* | 83.21% | 1.28% | 84.86% | 1.84% | 81.29% | 1.33% |
| 371* | 81.69% | 1.79% | 81.83% | 2.00% | 79.09% | 1.07% |
| 382* | 83.35% | 2.01% | 83.30% | 2.09% | 79.28% | 1.24% |
| 394* | 84.03% | 2.23% | 81.91% | 2.10% | 80.16% | 1.27% |
| 412* | 84.16% | 2.19% | 84.34% | 2.01% | 83.70% | 1.26% |
| 427* | 84.96% | 2.17% | 85.66% | 1.08% | 86.10% | 1.30% |
| 441* | 83.00% | 2.08% | 74.51% | 1.53% | 84.95% | 1.21% |
| 453* | 78.14% | 1.91% | 76.15% | 0.76% | 79.34% | 1.16% |
| 395* | 64.70% | 1.47% | 67.05% | 1.49% | 75.19% | 1.31% |
| 413* | 75.70% | 1.53% | 79.47% | 1.89% | 81.90% | 1.20% |
| 428* | 82.77% | 1.80% | 80.28% | 2.24% | 84.33% | 1.43% |
| 442* | 81.51% | 1.29% | 85.35% | 1.58% | 86.34% | 1.18% |
| 454* | 84.30% | 1.84% | 83.68% | 1.92% | 88.55% | 0.96% |
| 465* | 84.94% | 1.75% | 85.80% | 1.77% | 87.60% | 1.29% |
| 475* | 81.08% | 1.57% | 84.07% | 1.59% | 83.53% | 0.94% |
| 414* | 76.12% | 1.90% | 80.06% | 2.10% | 81.39% | 1.03% |
| 429* | 74.14% | 1.87% | 77.28% | 1.21% | 82.38% | 1.31% |
| 443* | 75.77% | 2.03% | 74.59% | 0.70% | 82.88% | 1.26% |
| 455* | 67.08% | 1.61% | 76.14% | 1.78% | 84.85% | 1.08% |
| 466* | 52.31% | 1.29% | 71.04% | 1.61% | 81.51% | 1.06% |
| 476* | 73.64% | 2.10% | 66.15% | 1.49% | 75.01% | 1.08% |
| 485* | 81.92% | 1.79% | 72.99% | 0.73% | 85.10% | 0.83% |
| 354 | 38.59% | 11.16% | 26.22% | 7.18% | 38.47% | 13.67% |
| 361 | 37.99% | 10.52% | 30.48% | 7.73% | 39.71% | 11.75% |

¹The indicated PEgRNA sequence contains, from 5′ to 3′, a Spacer, a gRNA core according to SEQ ID NO: 665, an RTT, a PBS, a Linker (AACATTGA; Sequence Number 671) and a 3′ hairpin motif (CGCGTCTCTACGTGGGGGCGCG; SEQ ID NO: 672). The PEgRNA used experimentally further contained 3′ mN*mN*mN*N and 5′mN*mN*mN* modifications, where m indicates that the nucleotide contains a 2′-O—Me modification and a * indicates a phosphorothioate bond. Please see table 13 below for information on the Spacer, RTT, and PBS sequences used in the indicated PEgRNAs.
²A * indicates that the indicated PEgRNA encodes an AGG-to-AGC nonsynonymous [A49G] PAM silencing edit; lack of a * indicates the PEgRNA encodes wild-type CLRNI sequence.
³The ngRNA used experimentally contained, from 5′ to 3′, the indicated Spacer sequence, a gRNA core according to SEQ ID NO: 665, and a 3′ UUUU sequence. The ngRNA further contained 3′ mN*mN*mN*N and 5′mN*mN*mN* modifications, where m indicates that the nucleotide contains a 2′-O—Me modification and a * indicates a phosphorothioate bond. PE2 indicates that no ngRNA was used.
4.

TABLE 12e
Prime Editing at a mutation site in HEK293T cells with a PEgRNA

| PEgRNA^{1,2} | ngRNA spacer³ Sequence Number: | | | | | |
|---|---|---|---|---|---|---|
| Sequence | 164 | | 150 | | 149 | |
| Number: | % Edit | % indel | % Edit | % indel | % Edit | % indel |
| 368 | 48.13% | 5.70% | 23.17% | 5.49% | 21.46% | 3.93% |
| 379 | 49.86% | 6.41% | 24.89% | 5.75% | 27.88% | 6.83% |
| 391 | 53.89% | 6.77% | 25.39% | 4.39% | 21.24% | 2.36% |
| 409 | 53.65% | 5.91% | 24.90% | 3.28% | 21.76% | 2.40% |
| 355* | 29.12% | 5.76% | 11.15% | 4.34% | 6.27% | 2.94% |
| 362* | 33.48% | 5.74% | 9.00% | 3.61% | 5.63% | 2.47% |
| 369* | 35.72% | 5.24% | 11.14% | 3.83% | 7.25% | 2.80% |
| 380* | 17.75% | 7.73% | 11.99% | 4.85% | 7.73% | 3.23% |
| 392* | 17.93% | 5.68% | 10.82% | 2.99% | 7.01% | 2.36% |
| 410* | 30.77% | 4.34% | 9.05% | 3.01% | 9.92% | 4.38% |
| 356* | 79.93% | 1.48% | 74.47% | 1.43% | 54.83% | 0.91% |
| 363* | 78.75% | 1.29% | 68.90% | 1.09% | 57.60% | 0.95% |
| 370* | 82.08% | 1.89% | 71.38% | 1.50% | 61.90% | 1.14% |
| 381* | 85.63% | 1.68% | 69.42% | 1.16% | 65.33% | 1.00% |
| 393* | 89.55% | 1.47% | 71.27% | 0.97% | 64.83% | 1.04% |
| 411* | 83.66% | 1.71% | 65.75% | 1.18% | 56.27% | 1.07% |
| 426* | 91.48% | 1.33% | 80.15% | 1.19% | 59.72% | 0.85% |
| 371* | 88.53% | 1.13% | 70.07% | 0.92% | 56.02% | 0.84% |
| 382* | 85.56% | 1.27% | 74.91% | 1.34% | 61.27% | 0.78% |
| 394* | 64.98% | 1.07% | 70.49% | 0.69% | 62.05% | 0.69% |
| 412* | 89.84% | 0.74% | 76.83% | 0.95% | 69.30% | 0.82% |
| 427* | 89.38% | 0.88% | 74.44% | 0.71% | 65.36% | 0.68% |
| 441* | 88.88% | 1.07% | 77.54% | 0.84% | 68.91% | 0.78% |
| 453* | 86.44% | 1.14% | 75.63% | 1.02% | 74.91% | 0.82% |
| 395* | 76.23% | 1.38% | 63.03% | 0.88% | 69.71% | 1.01% |
| 413* | 77.96% | 1.54% | 73.51% | 0.83% | 68.20% | 0.99% |
| 428* | 71.91% | 1.16% | 68.38% | 0.96% | 67.10% | 0.85% |
| 442* | 83.64% | 0.85% | 74.55% | 0.83% | 70.88% | 0.88% |
| 454* | 85.12% | 1.14% | 76.10% | 0.93% | 80.11% | 1.01% |
| 465* | 76.72% | 1.26% | 74.25% | 1.05% | 71.72% | 0.94% |
| 475* | 86.40% | 0.97% | 77.97% | 0.96% | 77.54% | 0.85% |
| 414* | 74.40% | 0.96% | 63.56% | 0.60% | 62.99% | 0.68% |
| 429* | 80.92% | 0.89% | 64.96% | 0.72% | 63.22% | 0.89% |
| 443* | 77.13% | 1.27% | 63.07% | 0.73% | 62.51% | 0.57% |
| 455* | 77.31% | 0.82% | 65.21% | 0.76% | 78.04% | 0.76% |
| 466* | 78.20% | 0.88% | 60.75% | 0.66% | 69.58% | 0.80% |
| 476* | 68.05% | 1.06% | 61.86% | 0.75% | 71.87% | 0.83% |
| 485* | 63.84% | 0.54% | 75.15% | 0.51% | 64.40% | 0.65% |
| 354 | 31.57% | 9.25% | 27.88% | 9.56% | 27.72% | 9.19% |
| 361 | 42.90% | 7.94% | 26.78% | 6.23% | 28.76% | 5.80% |

¹The indicated PEgRNA sequence contains, from 5′ to 3′, a Spacer, a gRNA core according to SEQ ID NO: 665, an RTT, a PBS, a Linker (AACATTGA; Sequence Number 671) and a 3′ hairpin motif (CGCGTCTCTACGTGGGGGCGCG; SEQ ID NO: 672). The PEgRNA used experimentally further contained 3′ mN*mN*mN*N and 5′mN*mN*mN* modifications, where m indicates that the nucleotide contains a 2′-O—Me modification and a * indicates a phosphorothioate bond. Please see table 13 below for information on the Spacer, RTT, and PBS sequences used in the indicated PEgRNAs.
²A * indicates that the indicated PEgRNA encodes an AGG-to-AGC nonsynonymous [A49G] PAM silencing edit; lack of a * indicates the PEgRNA encodes wild-type CLRNI sequence.
³The ngRNA used experimentally contained, from 5′ to 3′, the indicated Spacer sequence, a gRNA core according to SEQ ID NO: 665, and a 3′ UUUU sequence. The ngRNA further contained 3′ mN*mN*mN*N and 5′mN*mN*mN* modifications, where m indicates that the nucleotide contains a 2′-O—Me modification and a * indicates a phosphorothioate bond. PE2 indicates that no ngRNA was used.

TABLE 13
Prime Editing at a mutation site in HEK293T cells with a PEgRNA

| PEgRNA | Spacer | RTT | RTT | PBS | PBS |
|---|---|---|---|---|---|
| Sequence | Sequence | Sequence | Length | Sequence | Length |
| Number | Number | Number | (nt) | Number | (nt) |
| 368 | 4 | 23 | 12 | 14 | 12 |
| 379 | 4 | 23 | 12 | 15 | 13 |
| 391 | 4 | 23 | 12 | 16 | 14 |
| 409 | 4 | 23 | 12 | 17 | 15 |
| 355* | 4 | 26 | 12 | 12 | 10 |
| 362* | 4 | 26 | 12 | 13 | 11 |
| 369* | 4 | 26 | 12 | 14 | 12 |
| 380* | 4 | 26 | 12 | 15 | 13 |
| 392* | 4 | 26 | 12 | 16 | 14 |
| 410* | 4 | 26 | 12 | 17 | 15 |
| 356* | 4 | 33 | 13 | 11 | 9 |
| 363* | 4 | 33 | 13 | 12 | 10 |
| 370* | 4 | 33 | 13 | 13 | 11 |
| 381* | 4 | 33 | 13 | 14 | 12 |
| 393* | 4 | 33 | 13 | 15 | 13 |
| 411* | 4 | 33 | 13 | 16 | 14 |
| 426* | 4 | 33 | 13 | 17 | 15 |
| 371* | 4 | 42 | 15 | 11 | 9 |
| 382* | 4 | 42 | 15 | 12 | 10 |
| 394* | 4 | 42 | 15 | 13 | 11 |
| 412* | 4 | 42 | 15 | 14 | 12 |
| 427* | 4 | 42 | 15 | 15 | 13 |
| 441* | 4 | 42 | 15 | 16 | 14 |
| 453* | 4 | 42 | 15 | 17 | 15 |
| 395* | 4 | 51 | 17 | 11 | 9 |
| 413* | 4 | 51 | 17 | 12 | 10 |
| 428* | 4 | 51 | 17 | 13 | 11 |
| 442* | 4 | 51 | 17 | 14 | 12 |
| 454* | 4 | 51 | 17 | 15 | 13 |
| 465* | 4 | 51 | 17 | 16 | 14 |
| 475* | 4 | 51 | 17 | 17 | 15 |
| 414* | 4 | 57 | 18 | 11 | 9 |
| 429* | 4 | 57 | 18 | 12 | 10 |
| 443* | 4 | 57 | 18 | 13 | 11 |
| 455* | 4 | 57 | 18 | 14 | 12 |
| 466* | 4 | 57 | 18 | 15 | 13 |
| 476* | 4 | 57 | 18 | 16 | 14 |
| 485* | 4 | 57 | 18 | 17 | 15 |
| 354 | 4 | 23 | 12 | 12 | 10 |
| 361 | 4 | 23 | 12 | 13 | 11 |

1. The indicated PEgRNA sequence contains, from 5′ to 3′, the indicated Spacer sequence, a gRNA core according to SEQ ID NO: 665, the indicated RTT sequence, the indicated PBS sequence, a Linker (AACATTGA; Sequence Number 671) and a 3′ hairpin motif (CGCGTCTCTACGTGGGGGCGCG; SEQ ID NO: 672). The PEgRNA used experimentally further contained 3′ mN*mN*mN*N and 5′ mN*mN*mN* modifications, where m indicates that the nucleotide contains a 2′-O-Me modification and a * indicates a phosphorothioate bond.
2. A * indicates that the indicated PEgRNA encodes an AGG-to-AGC nonsynonymous [A49G] PAM silencing edit; lack of a * indicates the PEgRNA encodes wild-type CLRN1 sequence.

### EXAMPLE 5—Screening of PEgRNA and ngRNA for editing of a mutation associated with Usher Syndrome type 3

In this Example, a set of 24 PEgRNA from Example 4 are retested alone and with a subset of the ngRNA from Example 4 to confirm their editing efficiency.

The PEgRNAs used here were chemically synthesized by Integrated DNA Technologies (IDT). An HEK293T cell line carrying the N48K mutation generated according to Example 2 was expanded and transiently transfected with mRNA encoding a Prime Editor fusion protein and PEgRNA in arrayed 96-well plates for assessment of editing by high-throughput sequencing.

The results, shown in Tables 14a to 14b confirm the efficacy of these PEgRNA in both a PE2 and PE3 system.

TABLE 14a
Prime Editing at a mutation site in HEK293T
cells with a PEgRNA and ngRNA (PE3 system)

| | ngRNA spacer³ Sequence Number: | | | | | |
|---|---|---|---|---|---|---|
| PEgRNA^{1,2} | PE2 | | 152 | | 161 | |
| Sequence | % | % | % | % | % | % |
| Number: | Edit⁴ | indel⁴ | Edit⁴ | indel⁴ | Edit⁴ | indel⁴ |
| 366 | 45.95% | 5.01% | 60.14% | 3.61% | 64.58% | 1.62% |
| 381* | 77.16% | 1.22% | 87.91% | 1.21% | 59.68% | 0.94% |
| 393* | 78.72% | 1.29% | 88.12% | 1.13% | 40.29% | 0.48% |
| 411* | 75.45% | 1.19% | 87.24% | 1.20% | 48.58% | 0.74% |
| 426* | 75.46% | 0.95% | 88.64% | 0.86% | 52.64% | 0.59% |
| 371* | 70.29% | 0.77% | 82.97% | 0.64% | 46.37% | 0.55% |
| 382* | 72.00% | 0.78% | 85.88% | 0.85% | 51.56% | 0.62% |
| 394* | 73.76% | 0.99% | 83.23% | 0.71% | 56.19% | 0.69% |
| 412* | 77.95% | 0.76% | 87.74% | 0.90% | 57.81% | 0.58% |
| 427* | 79.97% | 0.81% | 87.06% | 0.59% | 64.02% | 0.69% |
| 441* | 82.11% | 0.67% | 81.82% | 0.65% | 76.14% | 0.69% |
| 453* | 82.58% | 0.95% | 88.07% | 0.89% | 82.20% | 0.92% |
| 388 | 45.98% | 4.92% | 89.79% | 3.82% | 46.05% | 5.09% |
| 413* | 74.49% | 1.05% | 83.89% | 1.01% | 76.29% | 1.08% |
| 428* | 76.17% | 1.14% | 86.81% | 0.96% | 77.65% | 0.98% |
| 442* | 79.95% | 0.77% | 90.70% | 1.09% | 82.48% | 1.09% |
| 454* | 80.73% | 0.97% | 89.75% | 1.14% | 81.91% | 0.90% |
| 465* | 79.83% | 0.87% | 88.85% | 0.93% | 81.26% | 0.81% |
| 475* | 78.46% | 0.72% | 90.14% | 0.80% | 81.39% | 0.58% |
| 429* | 70.92% | 0.84% | 80.38% | 0.84% | 74.96% | 0.76% |
| 443* | 72.85% | 0.66% | 80.73% | 0.76% | 77.49% | 0.78% |
| 455* | 73.87% | 0.67% | 76.79% | 0.73% | 78.30% | 0.85% |
| 466* | 75.01% | 0.76% | 72.28% | 0.69% | 75.48% | 0.83% |
| 485* | 86.30% | 0.76% | 70.74% | 0.54% | 40.81% | 0.26% |

¹The indicated PEgRNA sequence contains, from 5′ to 3′, a Spacer, a gRNA core according to SEQ ID NO: 665, an RTT, a PBS, a Linker (AACATTGA; Sequence Number 671) and a 3′ hairpin motif (CGCGTCTCTACGTGGGGGCGCG; SEQ ID NO: 672). The PEgRNA used experimentally further contained 3′ mN*mN*mN*N and 5′mN*mN*mN* modifications, where m indicates that the nucleotide contains a 2′-O—Me modification and a * indicates a phosphorothioate bond. Please see table 15 below for information on the Spacer, RTT, and PBS sequences used in the indicated PEgRNAs.
²A * indicates that the indicated PEgRNA encodes an AGG-to-AGC nonsynonymous [A49G] PAM silencing edit; lack of a * indicates the PEgRNA encodes wild-type CLRNI sequence.
³The ngRNA used experimentally contained, from 5′ to 3′, the indicated Spacer sequence, a gRNA core according to SEQ ID NO: 665, and a 3′ UUUU sequence. The ngRNA further contained 3′ mN*mN*mN*N and 5′mN*mN*mN* modifications, where m indicates that the nucleotide contains a 2′-O—Me modification and a * indicates a phosphorothioate bond. PE2 indicates that no ngRNA was used.
⁴Twelve non-transfection control replicates were performed which yielded an average percent correction of 0.56% with a standard deviation of 0.11% and an average percent indel of 0.04% with a standard deviation of 0.03%.

TABLE 14b
Prime Editing at a mutation site in HEK293T
cells with a PEgRNA and ngRNA (PE3 system)

| PEgRNA^{1,2} | ngRNA spacer³ Sequence Number: | |
|---|---|---|
| Sequence | 164 | 181 |

| Number: | % Edit⁴ | % indel⁴ | % Edit⁴ | % indel⁴ |
|---|---|---|---|---|
| 366 | 46.95% | 5.13% | 53.50% | 2.40% |
| 381* | 80.26% | 1.95% | 64.79% | 1.02% |
| 393* | 81.95% | 1.35% | 69.34% | 0.96% |
| 411* | 79.65% | 1.46% | 64.02% | 0.97% |
| 426* | 81.49% | 1.19% | 78.77% | 0.95% |
| 371* | 77.43% | 0.89% | 64.07% | 0.70% |
| 382* | 78.59% | 0.75% | 61.23% | 0.59% |
| 394* | 79.89% | 0.91% | 67.40% | 0.90% |
| 412* | 80.90% | 1.01% | 74.30% | 0.84% |
| 427* | 81.17% | 0.94% | 55.81% | 0.65% |
| 441* | 79.99% | 1.16% | 73.62% | 0.73% |
| 453* | 79.90% | 1.02% | 77.31% | 0.72% |
| 388 | 71.42% | 5.61% | 54.43% | 2.61% |
| 413* | 79.18% | 1.09% | 65.26% | 0.82% |
| 428* | 81.99% | 1.28% | 73.88% | 0.98% |
| 442* | 83.24% | 1.02% | 74.54% | 0.86% |
| 454* | 81.31% | 0.81% | 78.02% | 0.82% |
| 465* | 81.84% | 1.06% | 80.03% | 0.82% |
| 475* | 87.31% | 0.98% | 80.41% | 0.84% |
| 429* | 78.98% | 1.03% | 66.65% | 0.82% |
| 443* | 77.39% | 0.85% | 61.41% | 0.74% |
| 455* | 82.01% | 0.94% | 65.39% | 0.73% |
| 466* | 12.92% | 0.11% | 69.17% | 0.67% |
| 485* | 80.60% | 1.12% | 71.45% | 0.87% |

¹The indicated PEgRNA sequence contains, from 5′ to 3′, a Spacer, a gRNA core according to SEQ ID NO: 665, an RTT, a PBS, a Linker (AACATTGA; Sequence Number 671) and a 3′ hairpin motif (CGCGTCTCTACGTGGGGGCGCG; SEQ ID NO: 672). The PEgRNA used experimentally further contained 3′ mN*mN*mN*N and 5′ mN*mN*mN* modifications, where m indicates that the nucleotide contains a 2′-O-Me modification and a * indicates a phosphorothioate bond. Please see table 15 below for information on the Spacer, RTT, and PBS sequences used in the indicated PEgRNAs.
²A * indicates that the indicated PEgRNA encodes an AGG-to-AGC nonsynonymous [A49G] PAM silencing edit; lack of a * indicates the PEgRNA encodes wild-type CLRN1 sequence.
³The ngRNA used experimentally contained, from 5′ to 3′, the indicated Spacer sequence, a gRNA core according to SEQ ID NO: 665, and a 3′ UUUU sequence. The ngRNA further contained 3′ mN*mN*mN*N and 5′ mN*mN*mN* modifications, where m indicates that the nucleotide contains a 2′-O-Me modification and a * indicates a phosphorothioate bond. PE2 indicates that no ngRNA was used.
⁴Twelve non-transfection control replicates were performed which yielded an average percent correction of 0.56% with a standard deviation of 0.11% and an average percent indel of 0.04% with a standard deviation of 0.03%.

TABLE 15
Prime Editing at a mutation site in HEK293T cells with a PEgRNA

| PEgRNA | Spacer | RTT | RTT | PBS | PBS |
|---|---|---|---|---|---|
| Sequence | Sequence | Sequence | Length | Sequence | Length |
| Number | Number | Number | (nt) | Number | (nt) |
| 366 | 4 | 28 | 13 | 13 | 11 |
| 381* | 4 | 33 | 13 | 14 | 12 |
| 393* | 4 | 33 | 13 | 15 | 13 |
| 411* | 4 | 33 | 13 | 16 | 14 |
| 426* | 4 | 33 | 13 | 17 | 15 |
| 371* | 4 | 42 | 15 | 11 | 9 |
| 382* | 4 | 42 | 15 | 12 | 10 |
| 394* | 4 | 42 | 15 | 13 | 11 |
| 412* | 4 | 42 | 15 | 14 | 12 |
| 427* | 4 | 42 | 15 | 15 | 13 |
| 441* | 4 | 42 | 15 | 16 | 14 |
| 453* | 4 | 42 | 15 | 17 | 15 |
| 388 | 4 | 28 | 13 | 15 | 13 |
| 413* | 4 | 51 | 17 | 12 | 10 |
| 428* | 4 | 51 | 17 | 13 | 11 |
| 442* | 4 | 51 | 17 | 14 | 12 |
| 454* | 4 | 51 | 17 | 15 | 13 |
| 465* | 4 | 51 | 17 | 16 | 14 |
| 475* | 4 | 51 | 17 | 17 | 15 |
| 429* | 4 | 57 | 18 | 12 | 10 |
| 443* | 4 | 57 | 18 | 13 | 11 |
| 455* | 4 | 57 | 18 | 14 | 12 |
| 466* | 4 | 57 | 18 | 15 | 13 |
| 485* | 4 | 57 | 18 | 17 | 15 |

1. The indicated PEgRNA sequence contains, from 5′ to 3′, the indicated Spacer sequence, a gRNA core according to SEQ ID NO: 665, the indicated RTT sequence, the indicated PBS sequence, a Linker (AACATTGA; Sequence Number 671) and a 3′ hairpin motif (CGCGTCTCTACGTGGGGGCGCG; SEQ ID NO: 672). The PEgRNA used experimentally further contained 3′ mN*mN*mN*N and 5′ mN*mN*mN* modifications, where m indicates that the nucleotide contains a 2′-O-Me modification and a * indicates a phosphorothioate bond.
2. A * indicates that the indicated PEgRNA encodes an AGG-to-AGC nonsynonymous [A49G] PAM silencing edit; lack of a * indicates the PEgRNA encodes wild-type CLRN1 sequence.

### Example 6—Screening of PEgRNA for Editing of a Mutation Associated with Usher Syndrome type 3

In this Example, PEgRNA were used in a pilot experiment using an alternative transfection protocol. The 24 PEgRNA from Examples 4 and 5, as well as modified versions of these PEgRNA were employed here. All of the modified versions included a highly 2-O-methylated scaffold. A few of the modified PEgRNAs contained an additional “T” nucleotide at the 5′ end of the RTT, which may result in a synonymous or non-synonymous edit.

The PEgRNAs used here were chemically synthesized by Integrated DNA Technologies (IDT). An HEK293T cell line carrying the N48K mutation generated according to Example 2 was expanded and transiently transfected with mRNA encoding a Prime Editor fusion protein and PEgRNA in arrayed 96-well plates for assessment of editing by high-throughput sequencing.

The results are shown in Table 16. Successful Prime Editing was observed for each PEgRNA tested. The lower levels of editing observed here compared to Examples 4 and 5 may indicate that the alternative transfection protocol used is less efficient.

TABLE 16
Prime Editing at a mutation site in HEK293T cells with a PEgRNA (PE2 system)

| PERNA¹ | Spacer | RTT² | | PBS | | | |
|---|---|---|---|---|---|---|---|
| Sequence | Sequence | Sequence | RTT | Sequence | PBS | % | % |
| Number | Number | Number | Length | Number | Length | Edit³ | Indel³ |
| 371 | 4 | 42* | 15 | 11 | 9 | 18.60% | 0.15% |
| 382 | 4 | 42* | 15 | 12 | 10 | 13.20% | 0.17% |
| 413 | 4 | 51* | 17 | 12 | 10 | 10.70% | 0.10% |
| 429 | 4 | 57* | 18 | 12 | 10 | 9.41% | 0.14% |
| 394 | 4 | 42* | 15 | 13 | 11 | 12.99% | 0.14% |
| 428 | 4 | 51* | 17 | 13 | 11 | 13.38% | 0.18% |
| 443 | 4 | 57* | 18 | 13 | 11 | 11.99% | 0.17% |
| 381 | 4 | 33* | 13 | 14 | 12 | 16.02% | 0.27% |
| 412 | 4 | 42* | 15 | 14 | 12 | 11.97% | 0.18% |
| 442 | 4 | 51* | 17 | 14 | 12 | 21.66% | 0.19% |
| 455 | 4 | 57* | 18 | 14 | 12 | 19.62% | 0.21% |
| 393 | 4 | 33* | 13 | 15 | 13 | 14.36% | 0.21% |
| 427 | 4 | 42* | 15 | 15 | 13 | 14.35% | 0.09% |
| 454 | 4 | 51* | 17 | 15 | 13 | 16.13% | 0.10% |
| 466 | 4 | 57* | 18 | 15 | 13 | 8.72% | 0.05% |
| 411 | 4 | 33* | 13 | 16 | 14 | 11.37% | 0.11% |
| 441 | 4 | 42* | 15 | 16 | 14 | 8.38% | 0.08% |
| 465 | 4 | 51* | 17 | 16 | 14 | 14.49% | 0.21% |
| 426 | 4 | 33* | 13 | 17 | 15 | 14.24% | 0.21% |
| 453 | 4 | 42* | 15 | 17 | 15 | 18.11% | 0.15% |
| 475 | 4 | 51* | 17 | 17 | 15 | 15.79% | 0.09% |
| 485 | 4 | 57* | 18 | 17 | 15 | 19.33% | 0.17% |
| 366 | 4 | 28 | 13 | 13 | 11 | 12.17% | 0.71% |
| 388 | 4 | 28 | 13 | 15 | 13 | 15.23% | 0.71% |
| 371^{&} | 4 | 42* | 15 | 11 | 9 | 5.91% | 0.11% |
| 382^{&} | 4 | 42* | 15 | 12 | 10 | 7.43% | 0.11% |
| 415^{&} | 4 | 52** | 17 | 12 | 10 | 6.63% | 0.09% |
| 429^{&} | 4 | 57* | 18 | 12 | 10 | 5.71% | 0.10% |
| 394^{&} | 4 | 42 | 15 | 13 | 11 | 9.37% | 0.21% |
| 430^{&} | 4 | 52** | 17 | 13 | 11 | 18.26% | 0.25% |
| 443^{&} | 4 | 57* | 18 | 13 | 11 | 8.24% | 0.10% |
| 383^{&} | 4 | 32*** | 13 | 14 | 12 | 2.13% | 0.51% |
| 412^{&} | 4 | 42* | 15 | 14 | 12 | 24.18% | 0.41% |
| 444^{&} | 4 | 52** | 17 | 14 | 12 | 19.09% | 0.23% |
| 455^{&} | 4 | 57* | 18 | 14 | 12 | 10.57% | 0.10% |
| 396^{&} | 4 | 32*** | 13 | 15 | 13 | 1.05% | 0.23% |
| 427^{&} | 4 | 42* | 15 | 15 | 13 | 4.94% | 0.05% |
| 456^{&} | 4 | 52** | 17 | 15 | 13 | 8.56% | 0.07% |
| 466^{&} | 4 | 57* | 18 | 15 | 13 | 10.33% | 0.10% |
| 416^{&} | 4 | 32*** | 13 | 16 | 14 | 1.00% | 0.15% |
| 441^{&} | 4 | 42* | 15 | 16 | 14 | 14.19% | 0.21% |
| 467^{&} | 4 | 52** | 17 | 16 | 14 | 14.14% | 0.19% |
| 431^{&} | 4 | 32*** | 13 | 17 | 15 | 1.60% | 0.39% |
| 453^{&} | 4 | 42* | 15 | 17 | 15 | 21.79% | 0.38% |
| 477^{&} | 4 | 52** | 17 | 17 | 15 | 14.26% | 0.23% |
| 485^{&} | 4 | 57* | 18 | 17 | 15 | 6.32% | 0.08% |
| 372^{&} | 4 | 31**** | 13 | 13 | 11 | 3.96% | 0.26% |
| 397^{&} | 4 | 31** | 13 | 15 | 13 | 5.65% | 0.40% |

¹The indicated sequence contains, from 5′ to 3′, the indicated Spacer sequence, a gRNA core according to SEQ ID NO: 665, the indicated RTT sequence, the indicated PBS sequence, a Linker (AACATTGA; Sequence Number 671) and a 3′ hairpin motif (CGCGTCTCTACGTGGGGGCGCG; SEQ ID NO: 672). The PEgRNA used experimentally further contained 3′ mN*mN*mN*N and
5′mN*mN*mN* modifications, where m indicates that the nucleotide contains a 2′-O—Me modification and a * indicates a phosphorothioate bond. An “&” means that 41 out of 77 nucleotides in scaffold/gRNA core contain a 2′-O—Me modification.
²*= RTT encodes a non-synonymous AGG-to-ACG [A49G] PAM silencing edit;
**= RTT encodes a G-to-T synonymous edit at its 5′ end and a non-synonymous AGG-to-ACG [A49G] PAM silencing edit;
***= RTT encodes a G-to-T non-synonymous [V47F] edit at its 5′ end and a non-synonymous AGG-to-ACG [A49G] PAM silencing edit;
****= RTT encodes a G-to-T non-synonymous [V47F] edit at its 5′ end.
³Five non-transfection control replicates were performed which yielded an average percent correction of 0.09% with a standard deviation of 0.02% and an average percent indel of 0.01% with a standard deviation of 0.004%.

What is claimed is:

1. A prime editing guide RNA (PEgRNA) or a nucleic acid encoding the PEgRNA,
wherein the PEgRNA comprises:
a) a spacer that is complementary to a search target sequence on a first strand of a CLRN1 gene wherein the spacer comprises at its 3′ end SEQ ID NO: 1;
b) a gRNA core capable of binding to a Cas9 protein; and
c) an extension arm comprising:
i. an editing template that comprises a region of complementarity to an editing target sequence on a second strand of the CLRN1 gene, and
ii. a primer binding site (PBS) that comprises at its 5′ end a sequence that is a reverse complement of nucleotides 10-14 of SEQ ID NO: 1,
wherein the first strand and second strand are complementary to each other,
wherein the editing target sequence on the second strand comprises or is complementary to a portion of the CLRN1 gene comprising a c. 144 T->G substitution, and
wherein the editing template encodes or comprises a wild-type amino acid sequence of a Clarin 1 protein at the c. 144 T->G substitution.

2. A prime editing guide RNA (PEgRNA), or a nucleic acid encoding the PEgRNA, wherein the PEgRNA comprises:
a) a spacer comprising at its 3′ end SEQ ID NO: 1;
b) a gRNA core capable of binding to a Cas9 protein; and
c) an extension arm comprising:
i. an editing template comprising at its 3′ end any one of SEQ ID NOs: 22-26, and
ii. a primer binding site (PBS) comprising at its 5′ end a sequence that is a reverse complement of nucleotides 10-14 of SEQ ID NO: 1.

3. The PEgRNA of any one of claims 1 or 2, wherein the spacer comprises at its 3′ end any one of SEQ ID NOs: 2-6.

4. The PEgRNA of claim 3, wherein the spacer comprises at its 3′ end SEQ I) NO: 4.

5. The PEgRNA of any one of claims 1-4, wherein the editing template comprises SEQ ID NO: 22 at its 3′ end and encodes an AGG to ATG PAM silencing edit.

6. The PEgRNA of claim 5, wherein the editing template comprises at its 3′ end SEQ ID NO: 27, 34, 38, 43, 47, 53, 58, 62, 66, 70, 74, 78, 82, 86, 90, 94, 98,102,106, 110, 114, 118, 122, 126, 130, 134, 138, or 142.

7. The PEgRNA of any one of claims 1-4, wherein the editing template comprises SEQ ID NO: 23 at its 3′ end.

8. The PEgRNA of claim 7, wherein the editing template comprises at its 3′ end SEQ ID NO: 28, 31, 35, 39, 44, 48, 54, 59, 63, 67, 71, 75, 79, 83, 87, 91, 95, 99,103,107, 111, 115, 119, 123, 127, 131, 135, 139, or 143.

9. The PEgRNA of any one of claims 1-4, wherein the editing template comprises SEQ ID NO: 24 at its 3′ end and encodes an AGG-to-ACG PAM silencing edit.

10. The PEgRNA of claim 9, wherein the editing template comprises at its 3′ end SEQ ID NO: 29, 36, 40, 45, 49, 55, 60, 64, 68, 72, 76, 80, 84, 88, 92, 96,100,104, 108, 112, 116, 120, 124, 128, 132, 136, 140, or 144.

11. The PEgRNA of any one of claims 1-4, wherein the editing template comprises SEQ ID NO: 25 at its 3′ end and encodes an AGG-to-AAG PAM silencing edit.

12. The PEgRNA of claim 11, wherein the editing template comprises at its 3′ end SEQ ID NO: 30, 37, 41, 46, 50, 56, 61, 65, 69, 73, 77, 81, 85, 89, 93, 97,101,105, 109, 113, 117, 121, 125, 129, 133, 137, 141, or 145.

13. The PEgRNA of any one of claims 1-4, wherein the editing template comprises SEQ ID NO: 26 at its 3′ end and encodes a AGG-to-AGC PAM silencing edit.

14. The PEgRNA of claim 13, wherein the editing template comprises at its 3′ end SEQ ID NO: 32, 33, 42, 51, 52, or 57.

15. The PEgRNA of any one of claims 1-4, wherein the editing template comprises at its 3′ end any one of sequences set forth in SEQ ID NOs: 22 to 145.

16. The PEgRNA of any one of claims 1-15, wherein the editing template has a length of 40 nucleotides or less.

17. The PEgRNA of any one of claims 1-16, wherein the editing template has a length of 26 nucleotide or less.

18. The PEgRNA of claim 16 or claim 17, wherein the editing template is 12 to 26 nucleotides in length.

19. The PEgRNA of any one of claims 1-17, wherein the editing template has a length of 18 nucleotides or less.

20. The PEgRNA of claim 19, wherein the editing template is 12 to 18 nucleotides in length.

21. The PEgRNA of any one of claims 1-20, wherein the PBS comprises at its 5′end a sequence corresponding to sequence number 7.

22. The PEgRNA of claim 21, wherein the PBS comprises sequence number 8, 9, 10, 11, 12 (SEQ ID NO: 12), 13 (SEQ ID NO: 13), 14 (SEQ ID NO: 14), 15 (SEQ ID NO: 15), 16 (SEQ ID NO: 16), 17 (SEQ ID NO: 17), 18 (SEQ ID NO: 18), 19 (SEQ ID NO: 19), 20 (SEQ ID NO: 20), or 21 (SEQ ID NO: 21).

23. The PEgRNA of any one of claims 1-22, wherein the PBS has a length of 16 nucleotides or less.

24. The PEgRNA of claim 23, wherein the PBS is 8 to 16 nucleotides in length.

25. The PEgRNA of any one of claims 1-23, wherein the PBS has a length of 15 nucleotides or less.

26. The PEgRNA of claim 25, wherein the PBS is 9 to 15 nucleotides in length.

27. A prime editing guide RNA (PEgRNA) comprising:
a. a spacer comprising at its 3′ end any one of a PEgRNA spacer sequence as set forth in Table 1;
b. a gRNA core capable of binding to a Cas9 protein; and
c. an extension arm comprising:
i. an editing template comprising at its 3′ end any one of a RTT sequence as set forth in Table 1; and
ii. a primer binding site (PBS) comprising at its 5′ end any one of a PBS sequence as set forth in Table 1.

28. The PEgRNA of any one of claims 1-27, wherein the spacer of the PEgRNA is from 17 to 22 nucleotides in length.

29. The PEgRNA of any one of claims 1-28, wherein the spacer of the PEgRNA is 20 nucleotides in length.

30. The PEgRNA of any one of claims 1-29, wherein the gRNA core comprises any one of SEQ ID NOs: 665-669.

31. The PEgRNA of any one of claims 1-30, comprising from 5′ to 3′, the spacer, the gRNA core, the editing template, and the PBS.

32. The PEgRNA of claim 31, wherein the spacer, the gRNA core, the editing template, and the PBS form a contiguous sequence in a single molecule.

33. The PEgRNA of any one of claims 1-32, wherein the PEgRNA further comprises a linker sequence at the 3′ end.

34. The PEgRNA of claim 33, wherein the linker sequence comprises a sequence set forth at Sequence Number 671.

35. The PEgRNA of any one of claims 1-34, wherein the PEgRNA further comprises a hairpin motif at the 3′ end.

36. The PEgRNA of claim 35, wherein the hairpin motif comprises a sequence set forth at SEQ ID NO: 672.

37. The PEgRNA of any one of claims 1-36, further comprising 3′ mN*mN*mN*N and 5′mN*mN*mN* modifications, where m indicates that the nucleotide contains a 2′-O-Me modification and a * indicates the presence of a phosphorothioate bond.

38. The PEgRNA of any one of claims 1-37, further comprising 3′ mT*mT*mT*T and 5′mN*mN*mN* modifications, where m indicates that the nucleotide contains a 2′-O-Me modification, a * indicates the presence of a phosphorothioate bond, and a T indicates the presence of an additional uridine nucleotide.

39. The PEgRNA of any one of claims 1-38, wherein the editing template encodes a PAM silencing edit.

40. The PEgRNA of any one of claims 1-36, comprising a PEgRNA sequence selected from any one of SEQ ID NOs: 195-508.

41. A prime editing system comprising:
(a) the PEgRNA or the nucleic acid encoding the PEgRNA of any one of claims 1-40, and
(b) a ngRNA, or a nucleic acid encoding the ngRNA, wherein the ngRNA comprises:
(i) a spacer comprising at its 3′ end a sequence corresponding to nucleotides 4-20 of any one of SEQ ID NO: 146-194; and
(ii) an ngRNA core capable of binding a Cas9 protein.

42. The prime editing system of claim 41, wherein the spacer of the ngRNA comprises at its 3′ end nucleotides 3-20, 2-20, or 1-20 of any one of SEQ ID NO: 146-194.

43. The prime editing system of claim 41, wherein the spacer of the ngRNA comprises at its 3′ end any one of SEQ ID NOs: 146-194.

44. A prime editing system comprising:
(a) the prime editing guide RNA (PEgRNA) of any one of claims 1-32, or a nucleic acid encoding the PEgRNA; and optionally
(b) a nick guide RNA (ngRNA), or a nucleic acid encoding the ngRNA,
wherein the ngRNA comprises a spacer comprising at its 3′ end nucleotides 4-20 of any one of a ngRNA spacer sequence set forth in Table 1 and a gRNA core capable of binding to a Cas9 protein.

45. The prime editing system of claim 44, comprising the ngRNA.

46. The prime editing system of any one of claims 44 to 45, wherein the spacer of the ngRNA comprises at its 3′ end nucleotides 3-20, 2-20, or 1-20 of the ngRNA spacer sequence.

47. The prime editing system of any one of claims 44 or 46, wherein the spacer of the ngRNA comprises at its 3′ end the ngRNA spacer sequence.

48. The prime editing system of any one of claims 41-47, wherein the spacer of the ngRNA is from 17 to 22 nucleotides in length.

49. The prime editing system of any one of claims 41-48, wherein the spacer of the ngRNA is 20 nucleotides in length.

50. The prime editing system of any one of claims 41-49, wherein the gRNA core of the ngRNA comprises any one of SEQ ID NOs: 665-669.51 The prime editing system of any of claims 41-50, wherein the ngRNA comprises any one of the SEQ ID NOs: 509-588.

52. The prime editing system of any one of claims 41-51, further comprising:
(c) a prime editor comprising:
(i) a Cas9 nickase comprising a nuclease inactivating mutation in the HNH domain, or a nucleic acid encoding the Cas9 nickase, and
(ii) a reverse transcriptase, or a nucleic acid encoding the reverse transcriptase.

53. The prime editing system of claim 52, wherein the prime editor is a fusion protein.

54. The prime editing system of any one of claims 52 or 53, further comprising:
(c) an N-terminal extein comprising an N-terminal fragment of a prime editor fusion protein and an N-intein or a polynucleotide encoding the N-terminal extein; and
(d) a C-terminal extein comprising a C-terminal fragment of the prime editor fusion protein and a C-intein, or a polynucleotide encoding the C-terminal extein; wherein the N-intein and the C-intein of the N-terminal and C-terminal exteins are capable of self-excision to join the N-terminal fragment and the C-terminal fragment to form the prime editor fusion protein, and wherein the prime editor fusion protein comprises a Cas9 nickase and a reverse transcriptase.

55. A prime editing system comprising:
(a) the PEgRNA of any one of claims 1-40, or the nucleotide encoding the PEgRNA; and
(b) a prime editor comprising a Cas9 nickase comprising a nuclease inactivating mutation in the HNH domain, or a nucleic acid encoding the Cas9 nickase, and a reverse transcriptase, or a nucleic acid encoding the reverse transcriptase.

56. A prime editing system comprising:
(a) the PEgRNA of any one of claims 1-40, or the nucleotide encoding the PEgRNA;
(b) an N-terminal extein comprising an N-terminal fragment of a prime editor fusion protein and an N-intein or a polynucleotide encoding the N-terminal extein; and
(c) a C-terminal extein comprising a C-terminal fragment of the prime editor fusion protein and a C-intein, or a polynucleotide encoding the C-terminal extein; wherein the N-intein and the C-intein of the N-terminal and C-terminal exteins are capable of self-excision to join the N-terminal fragment and the C-terminal fragment to form the prime editor fusion protein, and wherein the prime editor fusion protein comprises a Cas9 nickase and a reverse transcriptase.

57. The prime editing system of any one of claims 52-56, wherein the Cas9 nickase comprises an amino acid sequence comprising at least 80%, 85%, 90%, 95%, 96%, 97%, 98%, 99%, or 100% identity to any one of SEQ ID NOs: 593, 594, 596, 597, 599, 600, 602, 603, 605, 606, 608, 609, 611, 612, 614, 615, 617, 618, or 619.

58. The prime editing system of any one of claims 52-57, wherein the reverse transcriptase comprises an amino acid sequence comprising at least 80%, 85%, 90%, 95%, 96%, 97%, 98%, 99%, or 100% identity to any one of SEQ ID NOs: 589, 590, or 591.

59. The prime editing system of claims 57 or 58, wherein the sequence identities are determined by Needleman-Wunsch alignment of two protein sequences with Gap Costs set to Existence: 11 Extension: 1 where percent identity is calculated by dividing the number of identities by a length of the alignment.

60. A population of viral particles collectively comprising the one or more nucleic acids encoding the prime editing system of any one of claims 41-59.

61. The population of viral particle of claim 60, wherein the viral particles are AAV particles.

62. An LNP comprising the prime editing system of any one of claims 41-59.

63. The LNP of claim 62, comprising the PEgRNA, the nucleic acid encoding the Cas9 nickase, and the nucleic acid encoding the reverse transcriptase.

64. The LNP of claim 63, wherein the nucleic acid encoding the Cas9 nickase and the nucleic acid encoding the reverse transcriptase are mRNA.

65. The LNP of claims 63 or 64, wherein the nucleic acid encoding the Cas9 nickase and the nucleic acid encoding the reverse transcriptase are the same molecule.

66. A method of correcting or editing a CLRN1 gene, the method comprising contacting the CLRN1 gene with:
(a) the PEgRNA of any one of claims 1-40 and a prime editor comprising a Cas9 nickase comprising a nuclease inactivating mutation in a HNH domain, and a reverse transcriptase; or
(b) the prime editing system of any one of claims 41-59.

67. The method of claim 66, wherein the CLRN1 gene is in a cell.

68. The method of claim 67, wherein the cell is a mammalian cell.

69. The method of claim 68, wherein the cell is a human cell.

70. The method of any one of claims 66-69, wherein the cell is a primary cell.

71. The method of any one of claims 66-70, wherein the cell is in a subject.

72. The method of claim 71, wherein the subject is a human.

73. The method of any one of claims 67-72, wherein the cell is from a subject having Usher Syndrome Type 3.

74. The method of any one of claims 66-73, wherein contacting the CLRN1 gene comprises contacting the cell with (i) the population of viral particles of claims 60 or 61; or
(ii) the LNP of any one of claims 62-65.

75. A method for treating Usher Syndrome Type 3 in a subject in need thereof, the method comprising administering to the subject:
(a) the PEgRNA of any one of claims 1-40 and a prime editor comprising a Cas9 nickase comprising a nuclease inactivating mutation in the HNH domain and a reverse transcriptase;
(b) the prime editing system of any one of claims 41-59;
(c) the population of viral particles of claims 60 or 61; or
(d) the LNP of any one of claims 62-65.


SEQUENCE LISTING (681 sequences)

SEQ ID NO: 1 - RNA, 17 nt, synthetic construct
  source 1..17: mol_type = other RNA
gtccagctcctgccctg

SEQ ID NO: 2 - RNA, 18 nt, synthetic construct
  source 1..18: mol_type = other RNA
tgtccagctcctgccctg

SEQ ID NO: 3 - RNA, 19 nt, synthetic construct
  source 1..19: mol_type = other RNA
ttgtccagctcctgccctg

SEQ ID NO: 4 - RNA, 20 nt, synthetic construct
  source 1..20: mol_type = other RNA
cttgtccagctcctgccctg

SEQ ID NO: 5 - RNA, 21 nt, synthetic construct
  source 1..21: mol_type = other RNA
acttgtccagctcctgccctg

SEQ ID NO: 6 - RNA, 22 nt, synthetic construct
  source 1..22: mol_type = other RNA
aacttgtccagctcctgccctg

SEQ ID NO: 7-11 - skipped (5 entries)

SEQ ID NO: 12 - RNA, 10 nt, synthetic construct
  source 1..10: mol_type = other RNA
ggcaggagct

SEQ ID NO: 13 - RNA, 11 nt, synthetic construct
  source 1..11: mol_type = other RNA
ggcaggagctg

SEQ ID NO: 14 - RNA, 12 nt, synthetic construct
  source 1..12: mol_type = other RNA
ggcaggagctgg

SEQ ID NO: 15 - RNA, 13 nt, synthetic construct
  source 1..13: mol_type = other RNA
ggcaggagctgga

SEQ ID NO: 16 - RNA, 14 nt, synthetic construct
  source 1..14: mol_type = other RNA
ggcaggagctggac

SEQ ID NO: 17 - RNA, 15 nt, synthetic construct
  source 1..15: mol_type = other RNA
ggcaggagctggaca

SEQ ID NO: 18 - RNA, 16 nt, synthetic construct
  source 1..16: mol_type = other RNA
ggcaggagctggacaa

SEQ ID NO: 19 - RNA, 17 nt, synthetic construct
  source 1..17: mol_type = other RNA
ggcaggagctggacaag

SEQ ID NO: 20 - RNA, 18 nt, synthetic construct
  source 1..18: mol_type = other RNA
ggcaggagctggacaagt

SEQ ID NO: 21 - RNA, 19 nt, synthetic construct
  source 1..19: mol_type = other RNA
ggcaggagctggacaagtt

SEQ ID NO: 22 - RNA, 12 nt, synthetic construct
  source 1..12: mol_type = other RNA
tcaatgcatcag

SEQ ID NO: 23 - RNA, 12 nt, synthetic construct
  source 1..12: mol_type = other RNA
tcaatgcctcag

SEQ ID NO: 24 - RNA, 12 nt, synthetic construct
  source 1..12: mol_type = other RNA
tcaatgcgtcag

SEQ ID NO: 25 - RNA, 12 nt, synthetic construct
  source 1..12: mol_type = other RNA
tcaatgcttcag

SEQ ID NO: 26 - RNA, 12 nt, synthetic construct
  source 1..12: mol_type = other RNA
tcaatggctcag

SEQ ID NO: 27 - RNA, 13 nt, synthetic construct
  source 1..13: mol_type = other RNA
gtcaatgcatcag

SEQ ID NO: 28 - RNA, 13 nt, synthetic construct
  source 1..13: mol_type = other RNA
gtcaatgcctcag

SEQ ID NO: 29 - RNA, 13 nt, synthetic construct
  source 1..13: mol_type = other RNA
gtcaatgcgtcag

SEQ ID NO: 30 - RNA, 13 nt, synthetic construct
  source 1..13: mol_type = other RNA
gtcaatgcttcag

SEQ ID NO: 31 - RNA, 13 nt, synthetic construct
  source 1..13: mol_type = other RNA
ttcaatgcctcag

SEQ ID NO: 32 - RNA, 13 nt, synthetic construct
  source 1..13: mol_type = other RNA
ttcaatggctcag

SEQ ID NO: 33 - RNA, 13 nt, synthetic construct
  source 1..13: mol_type = other RNA
gtcaatggctcag

SEQ ID NO: 34 - RNA, 14 nt, synthetic construct
  source 1..14: mol_type = other RNA
cgtcaatgcatcag

SEQ ID NO: 35 - RNA, 14 nt, synthetic construct
  source 1..14: mol_type = other RNA
cgtcaatgcctcag

SEQ ID NO: 36 - RNA, 14 nt, synthetic construct
  source 1..14: mol_type = other RNA
cgtcaatgcgtcag

SEQ ID NO: 37 - RNA, 14 nt, synthetic construct
  source 1..14: mol_type = other RNA
cgtcaatgcttcag

SEQ ID NO: 38 - RNA, 15 nt, synthetic construct
  source 1..15: mol_type = other RNA
tcgtcaatgcatcag

SEQ ID NO: 39 - RNA, 15 nt, synthetic construct
  source 1..15: mol_type = other RNA
tcgtcaatgcctcag

SEQ ID NO: 40 - RNA, 15 nt, synthetic construct
  source 1..15: mol_type = other RNA
tcgtcaatgcgtcag

SEQ ID NO: 41 - RNA, 15 nt, synthetic construct
  source 1..15: mol_type = other RNA
tcgtcaatgcttcag

SEQ ID NO: 42 - RNA, 15 nt, synthetic construct
  source 1..15: mol_type = other RNA
tcgtcaatggctcag

SEQ ID NO: 43 - RNA, 16 nt, synthetic construct
  source 1..16: mol_type = other RNA
ctcgtcaatgcatcag

SEQ ID NO: 44 - RNA, 16 nt, synthetic construct
  source 1..16: mol_type = other RNA
ctcgtcaatgcctcag

SEQ ID NO: 45 - RNA, 16 nt, synthetic construct
  source 1..16: mol_type = other RNA
ctcgtcaatgcgtcag

SEQ ID NO: 46 - RNA, 16 nt, synthetic construct
  source 1..16: mol_type = other RNA
ctcgtcaatgcttcag

SEQ ID NO: 47 - RNA, 17 nt, synthetic construct
  source 1..17: mol_type = other RNA
gctcgtcaatgcatcag

SEQ ID NO: 48 - RNA, 17 nt, synthetic construct
  source 1..17: mol_type = other RNA
gctcgtcaatgcctcag

SEQ ID NO: 49 - RNA, 17 nt, synthetic construct
  source 1..17: mol_type = other RNA
gctcgtcaatgcgtcag

SEQ ID NO: 50 - RNA, 17 nt, synthetic construct
  source 1..17: mol_type = other RNA
gctcgtcaatgcttcag

SEQ ID NO: 51 - RNA, 17 nt, synthetic construct
  source 1..17: mol_type = other RNA
gctcgtcaatggctcag

SEQ ID NO: 52 - RNA, 17 nt, synthetic construct
  source 1..17: mol_type = other RNA
tctcgtcaatggctcag

SEQ ID NO: 53 - RNA, 18 nt, synthetic construct
  source 1..18: mol_type = other RNA
tgctcgtcaatgcatcag

SEQ ID NO: 54 - RNA, 18 nt, synthetic construct
  source 1..18: mol_type = other RNA
tgctcgtcaatgcctcag

SEQ ID NO: 55 - RNA, 18 nt, synthetic construct
  source 1..18: mol_type = other RNA
tgctcgtcaatgcgtcag

SEQ ID NO: 56 - RNA, 18 nt, synthetic construct
  source 1..18: mol_type = other RNA
tgctcgtcaatgcttcag

SEQ ID NO: 57 - RNA, 18 nt, synthetic construct
  source 1..18: mol_type = other RNA
tgctcgtcaatggctcag

SEQ ID NO: 58 - RNA, 19 nt, synthetic construct
  source 1..19: mol_type = other RNA
ctgctcgtcaatgcatcag

SEQ ID NO: 59 - RNA, 19 nt, synthetic construct
  source 1..19: mol_type = other RNA
ctgctcgtcaatgcctcag

SEQ ID NO: 60 - RNA, 19 nt, synthetic construct
  source 1..19: mol_type = other RNA
ctgctcgtcaatgcgtcag

SEQ ID NO: 61 - RNA, 19 nt, synthetic construct
  source 1..19: mol_type = other RNA
ctgctcgtcaatgcttcag

SEQ ID NO: 62 - RNA, 20 nt, synthetic construct
  source 1..20: mol_type = other RNA
tctgctcgtcaatgcatcag

SEQ ID NO: 63 - RNA, 20 nt, synthetic construct
  source 1..20: mol_type = other RNA
tctgctcgtcaatgcctcag

SEQ ID NO: 64 - RNA, 20 nt, synthetic construct
  source 1..20: mol_type = other RNA
tctgctcgtcaatgcgtcag

SEQ ID NO: 65 - RNA, 20 nt, synthetic construct
  source 1..20: mol_type = other RNA
tctgctcgtcaatgcttcag

SEQ ID NO: 66 - RNA, 21 nt, synthetic construct
  source 1..21: mol_type = other RNA
ctctgctcgtcaatgcatcag

SEQ ID NO: 67 - RNA, 21 nt, synthetic construct
  source 1..21: mol_type = other RNA
ctctgctcgtcaatgcctcag

SEQ ID NO: 68 - RNA, 21 nt, synthetic construct
  source 1..21: mol_type = other RNA
ctctgctcgtcaatgcgtcag

SEQ ID NO: 69 - RNA, 21 nt, synthetic construct
  source 1..21: mol_type = other RNA
ctctgctcgtcaatgcttcag

SEQ ID NO: 70 - RNA, 22 nt, synthetic construct
  source 1..22: mol_type = other RNA
gctctgctcgtcaatgcatcag

SEQ ID NO: 71 - RNA, 22 nt, synthetic construct
  source 1..22: mol_type = other RNA
gctctgctcgtcaatgcctcag

SEQ ID NO: 72 - RNA, 22 nt, synthetic construct
  source 1..22: mol_type = other RNA
gctctgctcgtcaatgcgtcag

SEQ ID NO: 73 - RNA, 22 nt, synthetic construct
  source 1..22: mol_type = other RNA
gctctgctcgtcaatgcttcag

SEQ ID NO: 74 - RNA, 23 nt, synthetic construct
  source 1..23: mol_type = other RNA
agctctgctcgtcaatgcatcag

SEQ ID NO: 75 - RNA, 23 nt, synthetic construct
  source 1..23: mol_type = other RNA
agctctgctcgtcaatgcctcag

SEQ ID NO: 76 - RNA, 23 nt, synthetic construct
  source 1..23: mol_type = other RNA
agctctgctcgtcaatgcgtcag

SEQ ID NO: 77 - RNA, 23 nt, synthetic construct
  source 1..23: mol_type = other RNA
agctctgctcgtcaatgcttcag

SEQ ID NO: 78 - RNA, 24 nt, synthetic construct
  source 1..24: mol_type = other RNA
gagctctgctcgtcaatgcatcag

SEQ ID NO: 79 - RNA, 24 nt, synthetic construct
  source 1..24: mol_type = other RNA
gagctctgctcgtcaatgcctcag

SEQ ID NO: 80 - RNA, 24 nt, synthetic construct
  source 1..24: mol_type = other RNA
gagctctgctcgtcaatgcgtcag

SEQ ID NO: 81 - RNA, 24 nt, synthetic construct
  source 1..24: mol_type = other RNA
gagctctgctcgtcaatgcttcag

SEQ ID NO: 82 - RNA, 25 nt, synthetic construct
  source 1..25: mol_type = other RNA
ggagctctgctcgtcaatgcatcag

SEQ ID NO: 83 - RNA, 25 nt, synthetic construct
  source 1..25: mol_type = other RNA
ggagctctgctcgtcaatgcctcag

SEQ ID NO: 84 - RNA, 25 nt, synthetic construct
  source 1..25: mol_type = other RNA
ggagctctgctcgtcaatgcgtcag

SEQ ID NO: 85 - RNA, 25 nt, synthetic construct
  source 1..25: mol_type = other RNA
ggagctctgctcgtcaatgcttcag

SEQ ID NO: 86 - RNA, 26 nt, synthetic construct
  source 1..26: mol_type = other RNA
gggagctctgctcgtcaatgcatcag

SEQ ID NO: 87 - RNA, 26 nt, synthetic construct
  source 1..26: mol_type = other RNA
gggagctctgctcgtcaatgcctcag

SEQ ID NO: 88 - RNA, 26 nt, synthetic construct
  source 1..26: mol_type = other RNA
gggagctctgctcgtcaatgcgtcag

SEQ ID NO: 89 - RNA, 26 nt, synthetic construct
  source 1..26: mol_type = other RNA
gggagctctgctcgtcaatgcttcag

SEQ ID NO: 90 - RNA, 27 nt, synthetic construct
  source 1..27: mol_type = other RNA
cgggagctctgctcgtcaatgcatcag

SEQ ID NO: 91 - RNA, 27 nt, synthetic construct
  source 1..27: mol_type = other RNA
cgggagctctgctcgtcaatgcctcag

SEQ ID NO: 92 - RNA, 27 nt, synthetic construct
  source 1..27: mol_type = other RNA
cgggagctctgctcgtcaatgcgtcag

SEQ ID NO: 93 - RNA, 27 nt, synthetic construct
  source 1..27: mol_type = other RNA
cgggagctctgctcgtcaatgcttcag

SEQ ID NO: 94 - RNA, 28 nt, synthetic construct
  source 1..28: mol_type = other RNA
acgggagctctgctcgtcaatgcatcag

SEQ ID NO: 95 - RNA, 28 nt, synthetic construct
  source 1..28: mol_type = other RNA
acgggagctctgctcgtcaatgcctcag

SEQ ID NO: 96 - RNA, 28 nt, synthetic construct
  source 1..28: mol_type = other RNA
acgggagctctgctcgtcaatgcgtcag

SEQ ID NO: 97 - RNA, 28 nt, synthetic construct
  source 1..28: mol_type = other RNA
acgggagctctgctcgtcaatgcttcag

SEQ ID NO: 98 - RNA, 29 nt, synthetic construct
  source 1..29: mol_type = other RNA
aacgggagctctgctcgtcaatgcatcag

SEQ ID NO: 99 - RNA, 29 nt, synthetic construct
  source 1..29: mol_type = other RNA
aacgggagctctgctcgtcaatgcctcag

SEQ ID NO: 100 - RNA, 29 nt, synthetic construct
  source 1..29: mol_type = other RNA
aacgggagctctgctcgtcaatgcgtcag

SEQ ID NO: 101 - RNA, 29 nt, synthetic construct
  source 1..29: mol_type = other RNA
aacgggagctctgctcgtcaatgcttcag

SEQ ID NO: 102 - RNA, 30 nt, synthetic construct
  source 1..30: mol_type = other RNA
aaacgggagctctgctcgtcaatgcatcag

SEQ ID NO: 103 - RNA, 30 nt, synthetic construct
  source 1..30: mol_type = other RNA
aaacgggagctctgctcgtcaatgcctcag

SEQ ID NO: 104 - RNA, 30 nt, synthetic construct
  source 1..30: mol_type = other RNA
aaacgggagctctgctcgtcaatgcgtcag

SEQ ID NO: 105 - RNA, 30 nt, synthetic construct
  source 1..30: mol_type = other RNA
aaacgggagctctgctcgtcaatgcttcag

SEQ ID NO: 106 - RNA, 31 nt, synthetic construct
  source 1..31: mol_type = other RNA
aaaacgggagctctgctcgtcaatgcatcag

SEQ ID NO: 107 - RNA, 31 nt, synthetic construct
  source 1..31: mol_type = other RNA
aaaacgggagctctgctcgtcaatgcctcag

SEQ ID NO: 108 - RNA, 31 nt, synthetic construct
  source 1..31: mol_type = other RNA
aaaacgggagctctgctcgtcaatgcgtcag

SEQ ID NO: 109 - RNA, 31 nt, synthetic construct
  source 1..31: mol_type = other RNA
aaaacgggagctctgctcgtcaatgcttcag

SEQ ID NO: 110 - RNA, 32 nt, synthetic construct
  source 1..32: mol_type = other RNA
caaaacgggagctctgctcgtcaatgcatcag

SEQ ID NO: 111 - RNA, 32 nt, synthetic construct
  source 1..32: mol_type = other RNA
caaaacgggagctctgctcgtcaatgcctcag

SEQ ID NO: 112 - RNA, 32 nt, synthetic construct
  source 1..32: mol_type = other RNA
caaaacgggagctctgctcgtcaatgcgtcag

SEQ ID NO: 113 - RNA, 32 nt, synthetic construct
  source 1..32: mol_type = other RNA
caaaacgggagctctgctcgtcaatgcttcag

SEQ ID NO: 114 - RNA, 33 nt, synthetic construct
  source 1..33: mol_type = other RNA
gcaaaacgggagctctgctcgtcaatgcatcag

SEQ ID NO: 115 - RNA, 33 nt, synthetic construct
  source 1..33: mol_type = other RNA
gcaaaacgggagctctgctcgtcaatgcctcag

SEQ ID NO: 116 - RNA, 33 nt, synthetic construct
  source 1..33: mol_type = other RNA
gcaaaacgggagctctgctcgtcaatgcgtcag

SEQ ID NO: 117 - RNA, 33 nt, synthetic construct
  source 1..33: mol_type = other RNA
gcaaaacgggagctctgctcgtcaatgcttcag

SEQ ID NO: 118 - RNA, 34 nt, synthetic construct
  source 1..34: mol_type = other RNA
tgcaaaacgggagctctgctcgtcaatgcatcag

SEQ ID NO: 119 - RNA, 34 nt, synthetic construct
  source 1..34: mol_type = other RNA
tgcaaaacgggagctctgctcgtcaatgcctcag

SEQ ID NO: 120 - RNA, 34 nt, synthetic construct
  source 1..34: mol_type = other RNA
tgcaaaacgggagctctgctcgtcaatgcgtcag

SEQ ID NO: 121 - RNA, 34 nt, synthetic construct
  source 1..34: mol_type = other RNA
tgcaaaacgggagctctgctcgtcaatgcttcag

SEQ ID NO: 122 - RNA, 35 nt, synthetic construct
  source 1..35: mol_type = other RNA
ctgcaaaacgggagctctgctcgtcaatgcatcag

SEQ ID NO: 123 - RNA, 35 nt, synthetic construct
  source 1..35: mol_type = other RNA
ctgcaaaacgggagctctgctcgtcaatgcctcag

SEQ ID NO: 124 - RNA, 35 nt, synthetic construct
  source 1..35: mol_type = other RNA
ctgcaaaacgggagctctgctcgtcaatgcgtcag

SEQ ID NO: 125 - RNA, 35 nt, synthetic construct
  source 1..35: mol_type = other RNA
ctgcaaaacgggagctctgctcgtcaatgcttcag

SEQ ID NO: 126 - RNA, 36 nt, synthetic construct
  source 1..36: mol_type = other RNA
tctgcaaaacgggagctctgctcgtcaatgcatcag

SEQ ID NO: 127 - RNA, 36 nt, synthetic construct
  source 1..36: mol_type = other RNA
tctgcaaaacgggagctctgctcgtcaatgcctcag

SEQ ID NO: 128 - RNA, 36 nt, synthetic construct
  source 1..36: mol_type = other RNA
tctgcaaaacgggagctctgctcgtcaatgcgtcag

SEQ ID NO: 129 - RNA, 36 nt, synthetic construct
  source 1..36: mol_type = other RNA
tctgcaaaacgggagctctgctcgtcaatgcttcag

SEQ ID NO: 130 - RNA, 37 nt, synthetic construct
  source 1..37: mol_type = other RNA
ctctgcaaaacgggagctctgctcgtcaatgcatcag

SEQ ID NO: 131 - RNA, 37 nt, synthetic construct
  source 1..37: mol_type = other RNA
ctctgcaaaacgggagctctgctcgtcaatgcctcag

SEQ ID NO: 132 - RNA, 37 nt, synthetic construct
  source 1..37: mol_type = other RNA
ctctgcaaaacgggagctctgctcgtcaatgcgtcag

SEQ ID NO: 133 - RNA, 37 nt, synthetic construct
  source 1..37: mol_type = other RNA
ctctgcaaaacgggagctctgctcgtcaatgcttcag

SEQ ID NO: 134 - RNA, 38 nt, synthetic construct
  source 1..38: mol_type = other RNA
cctctgcaaaacgggagctctgctcgtcaatgcatcag

SEQ ID NO: 135 - RNA, 38 nt, synthetic construct
  source 1..38: mol_type = other RNA
cctctgcaaaacgggagctctgctcgtcaatgcctcag

SEQ ID NO: 136 - RNA, 38 nt, synthetic construct
  source 1..38: mol_type = other RNA
cctctgcaaaacgggagctctgctcgtcaatgcgtcag

SEQ ID NO: 137 - RNA, 38 nt, synthetic construct
  source 1..38: mol_type = other RNA
cctctgcaaaacgggagctctgctcgtcaatgcttcag

SEQ ID NO: 138 - RNA, 39 nt, synthetic construct
  source 1..39: mol_type = other RNA
tcctctgcaaaacgggagctctgctcgtcaatgcatcag

SEQ ID NO: 139 - RNA, 39 nt, synthetic construct
  source 1..39: mol_type = other RNA
tcctctgcaaaacgggagctctgctcgtcaatgcctcag

SEQ ID NO: 140 - RNA, 39 nt, synthetic construct
  source 1..39: mol_type = other RNA
tcctctgcaaaacgggagctctgctcgtcaatgcgtcag

SEQ ID NO: 141 - RNA, 39 nt, synthetic construct
  source 1..39: mol_type = other RNA
tcctctgcaaaacgggagctctgctcgtcaatgcttcag

SEQ ID NO: 142 - RNA, 40 nt, synthetic construct
  source 1..40: mol_type = other RNA
gtcctctgcaaaacgggagctctgctcgtcaatgcatcag

SEQ ID NO: 143 - RNA, 40 nt, synthetic construct
  source 1..40: mol_type = other RNA
gtcctctgcaaaacgggagctctgctcgtcaatgcctcag

SEQ ID NO: 144 - RNA, 40 nt, synthetic construct
  source 1..40: mol_type = other RNA
gtcctctgcaaaacgggagctctgctcgtcaatgcgtcag

SEQ ID NO: 145 - RNA, 40 nt, synthetic construct
  source 1..40: mol_type = other RNA
gtcctctgcaaaacgggagctctgctcgtcaatgcttcag

SEQ ID NO: 146 - RNA, 20 nt, synthetic construct
  source 1..20: mol_type = other RNA
aacgggagctctgctcgtca

SEQ ID NO: 147 - RNA, 20 nt, synthetic construct
  source 1..20: mol_type = other RNA
agccactgtcctctgcaaaa

SEQ ID NO: 148 - RNA, 20 nt, synthetic construct
  source 1..20: mol_type = other RNA
cagccttggggacaccgttg

SEQ ID NO: 149 - RNA, 20 nt, synthetic construct
  source 1..20: mol_type = other RNA
cctcggagttgtgacagcct

SEQ ID NO: 150 - RNA, 20 nt, synthetic construct
  source 1..20: mol_type = other RNA
ctcggagttgtgacagcctt

SEQ ID NO: 151 - RNA, 20 nt, synthetic construct
  source 1..20: mol_type = other RNA
ctctgctcgtcaatgcatca

SEQ ID NO: 152 - RNA, 20 nt, synthetic construct
  source 1..20: mol_type = other RNA
ctctgctcgtcaatgcctca

SEQ ID NO: 153 - RNA, 20 nt, synthetic construct
  source 1..20: mol_type = other RNA
ctctgctcgtcaatgcgtca

SEQ ID NO: 154 - RNA, 20 nt, synthetic construct
  source 1..20: mol_type = other RNA
ctctgctcgtcaatgcttca

SEQ ID NO: 155 - RNA, 20 nt, synthetic construct
  source 1..20: mol_type = other RNA
gccactgtcctctgcaaaac

SEQ ID NO: 156 - RNA, 20 nt, synthetic construct
  source 1..20: mol_type = other RNA
gctcgtcaatgcatcagggc

SEQ ID NO: 157 - RNA, 20 nt, synthetic construct
  source 1..20: mol_type = other RNA
gctcgtcaatgcctcagggc

SEQ ID NO: 158 - RNA, 20 nt, synthetic construct
  source 1..20: mol_type = other RNA
gctcgtcaatgcgtcagggc

SEQ ID NO: 159 - RNA, 20 nt, synthetic construct
  source 1..20: mol_type = other RNA
gctcgtcaatgcttcagggc

SEQ ID NO: 160 - RNA, 20 nt, synthetic construct
  source 1..20: mol_type = other RNA
gctctgctcgtcaatgcatc

SEQ ID NO: 161 - RNA, 20 nt, synthetic construct
  source 1..20: mol_type = other RNA
gctctgctcgtcaatgcctc

SEQ ID NO: 162 - RNA, 20 nt, synthetic construct
  source 1..20: mol_type = other RNA
gctctgctcgtcaatgcgtc

SEQ ID NO: 163 - RNA, 20 nt, synthetic construct
  source 1..20: mol_type = other RNA
gctctgctcgtcaatgcttc

SEQ ID NO: 164 - RNA, 20 nt, synthetic construct
  source 1..20: mol_type = other RNA
tcggagttgtgacagccttg

SEQ ID NO: 165 - RNA, 20 nt, synthetic construct
  source 1..20: mol_type = other RNA
ttcagttttgcatgtgccct

SEQ ID NO: 166 - RNA, 20 nt, synthetic construct
  source 1..20: mol_type = other RNA
gtacgggcttttccacggag

SEQ ID NO: 167 - RNA, 20 nt, synthetic construct
  source 1..20: mol_type = other RNA
ccaaggctgtcacaactccg

SEQ ID NO: 168 - RNA, 20 nt, synthetic construct
  source 1..20: mol_type = other RNA
tacgggcttttccacggaga

SEQ ID NO: 169 - RNA, 20 nt, synthetic construct
  source 1..20: mol_type = other RNA
gctctgctcgtcaaggcctc

SEQ ID NO: 170 - RNA, 20 nt, synthetic construct
  source 1..20: mol_type = other RNA
atgcagtacgggcttttcca

SEQ ID NO: 171 - RNA, 20 nt, synthetic construct
  source 1..20: mol_type = other RNA
caggagctggacaagtttat

SEQ ID NO: 172 - RNA, 20 nt, synthetic construct
  source 1..20: mol_type = other RNA
ctacttacatgagaaccgaa

SEQ ID NO: 173 - RNA, 20 nt, synthetic construct
  source 1..20: mol_type = other RNA
caatgcctcagggcaggagc

SEQ ID NO: 174 - RNA, 20 nt, synthetic construct
  source 1..20: mol_type = other RNA
ggttgggagcaaggcccttt

SEQ ID NO: 175 - RNA, 20 nt, synthetic construct
  source 1..20: mol_type = other RNA
gagagggtgtgaggcagtgt

SEQ ID NO: 176 - RNA, 20 nt, synthetic construct
  source 1..20: mol_type = other RNA
tttatgggtgaaatgcagta

SEQ ID NO: 177 - RNA, 20 nt, synthetic construct
  source 1..20: mol_type = other RNA
gggtgtgaggcagtgtgggt

SEQ ID NO: 178 - RNA, 20 nt, synthetic construct
  source 1..20: mol_type = other RNA
tacttacatgagaaccgaaa

SEQ ID NO: 179 - RNA, 20 nt, synthetic construct
  source 1..20: mol_type = other RNA
ggagagggtgtgaggcagtg

SEQ ID NO: 180 - RNA, 20 nt, synthetic construct
  source 1..20: mol_type = other RNA
gcaggagctggacaagttta

SEQ ID NO: 181 - RNA, 20 nt, synthetic construct
  source 1..20: mol_type = other RNA
ctctgctcgtcaatggctca

SEQ ID NO: 182 - RNA, 20 nt, synthetic construct
  source 1..20: mol_type = other RNA
ttatgggtgaaatgcagtac

SEQ ID NO: 183 - RNA, 20 nt, synthetic construct
  source 1..20: mol_type = other RNA
ggcagtgtgggttgggagca

SEQ ID NO: 184 - RNA, 20 nt, synthetic construct
  source 1..20: mol_type = other RNA
gctcgtcaaggcctcagggc

SEQ ID NO: 185 - RNA, 20 nt, synthetic construct
  source 1..20: mol_type = other RNA
actgcctcacaccctctccg

SEQ ID NO: 186 - RNA, 20 nt, synthetic construct
  source 1..20: mol_type = other RNA
gctcgtcaatggctcagggc

SEQ ID NO: 187 - RNA, 20 nt, synthetic construct
  source 1..20: mol_type = other RNA
ttttccacggagagggtgtg

SEQ ID NO: 188 - RNA, 20 nt, synthetic construct
  source 1..20: mol_type = other RNA
caaggcctcagggcaggagc

SEQ ID NO: 189 - RNA, 20 nt, synthetic construct
  source 1..20: mol_type = other RNA
gatccacaacggtgtcccca

SEQ ID NO: 190 - RNA, 20 nt, synthetic construct
  source 1..20: mol_type = other RNA
acagtggctttgatccacaa

SEQ ID NO: 191 - RNA, 20 nt, synthetic construct
  source 1..20: mol_type = other RNA
acatgcaaaactgaacactc

SEQ ID NO: 192 - RNA, 20 nt, synthetic construct
  source 1..20: mol_type = other RNA
ctctgctcgtcaaggcctca

SEQ ID NO: 193 - RNA, 20 nt, synthetic construct
  source 1..20: mol_type = other RNA
caaggctgtcacaactccga

SEQ ID NO: 194 - RNA, 20 nt, synthetic construct
  source 1..20: mol_type = other RNA
ggtgtgaggcagtgtgggtt

SEQ ID NO: 195 - RNA, 117 nt, synthetic construct
  source 1..117: mol_type = other RNA
cttgtccagctcctgccctggttttagagctagaaatagcaagttaaaataaggctagtccgttatcaacttgaaaaagtggcaccgagtcggtgcgtcaatgcctcagggcaggag

SEQ ID NO: 196 - RNA, 118 nt, synthetic construct
  source 1..118: mol_type = other RNA
cttgtccagctcctgccctggttttagagctagaaatagcaagttaaaataaggctagtccgttatcaacttgaaaaagtggcaccgagtcggtgcgtcaatgcctcagggcaggagc

SEQ ID NO: 197 - RNA, 118 nt, synthetic construct
  source 1..118: mol_type = other RNA
cttgtccagctcctgccctggttttagagctagaaatagcaagttaaaataaggctagtccgttatcaacttgaaaaagtggcaccgagtcggtgctcaatgcctcagggcaggagct

SEQ ID NO: 198 - RNA, 118 nt, synthetic construct
  source 1..118: mol_type = other RNA
cttgtccagctcctgccctggttttagagctagaaatagcaagttaaaataaggctagtccgttatcaacttgaaaaagtggcaccgagtcggtgctcaatggctcagggcaggagct

SEQ ID NO: 199 - RNA, 118 nt, synthetic construct
  source 1..118: mol_type = other RNA
cttgtccagctcctgccctggttttagagctagaaatagcaagttaaaataaggctagtccgttatcaacttgaaaaagtggcaccgagtcggtgcgtcaatggctcagggcaggagc

SEQ ID NO: 200 - RNA, 118 nt, synthetic construct
  source 1..118: mol_type = other RNA
cttgtccagctcctgccctggttttagagctagaaatagcaagttaaaataaggctagtccgttatcaacttgaaaaagtggcaccgagtcggtgctcaatgcgtcagggcaggagct

SEQ ID NO: 201 - RNA, 118 nt, synthetic construct
  source 1..118: mol_type = other RNA
cttgtccagctcctgccctggttttagagctagaaatagcaagttaaaataaggctagtccgttatcaacttgaaaaagtggcaccgagtcggtgcgtcaatgcgtcagggcaggagc

SEQ ID NO: 202 - RNA, 119 nt, synthetic construct
  source 1..119: mol_type = other RNA
cttgtccagctcctgccctggttttagagctagaaatagcaagttaaaataaggctagtccgttatcaacttgaaaaagtggcaccgagtcggtgctcgtcaatgcctcagggcaggag

SEQ ID NO: 203 - RNA, 119 nt, synthetic construct
  source 1..119: mol_type = other RNA
cttgtccagctcctgccctggttttagagctagaaatagcaagttaaaataaggctagtccgttatcaacttgaaaaagtggcaccgagtcggtgcgtcaatgcctcagggcaggagct

SEQ ID NO: 204 - RNA, 119 nt, synthetic construct
  source 1..119: mol_type = other RNA
cttgtccagctcctgccctggttttagagctagaaatagcaagttaaaataaggctagtccgttatcaacttgaaaaagtggcaccgagtcggtgctcaatgcctcagggcaggagctg

SEQ ID NO: 205 - RNA, 119 nt, synthetic construct
  source 1..119: mol_type = other RNA
cttgtccagctcctgccctggttttagagctagaaatagcaagttaaaataaggctagtccgttatcaacttgaaaaagtggcaccgagtcggtgctcaatggctcagggcaggagctg

SEQ ID NO: 206 - RNA, 119 nt, synthetic construct
  source 1..119: mol_type = other RNA
cttgtccagctcctgccctggttttagagctagaaatagcaagttaaaataaggctagtccgttatcaacttgaaaaagtggcaccgagtcggtgcgtcaatggctcagggcaggagct

SEQ ID NO: 207 - RNA, 119 nt, synthetic construct
  source 1..119: mol_type = other RNA
cttgtccagctcctgccctggttttagagctagaaatagcaagttaaaataaggctagtccgttatcaacttgaaaaagtggcaccgagtcggtgctcaatgcgtcagggcaggagctg

SEQ ID NO: 208 - RNA, 119 nt, synthetic construct
  source 1..119: mol_type = other RNA
cttgtccagctcctgccctggttttagagctagaaatagcaagttaaaataaggctagtccgttatcaacttgaaaaagtggcaccgagtcggtgcgtcaatgcgtcagggcaggagct

SEQ ID NO: 209 - RNA, 120 nt, synthetic construct
  source 1..120: mol_type = other RNA
cttgtccagctcctgccctggttttagagctagaaatagcaagttaaaataaggctagtccgttatcaacttgaaaaagtggcaccgagtcggtgcgtcaatgcctcagggcaggagctg

SEQ ID NO: 210 - RNA, 120 nt, synthetic construct
  source 1..120: mol_type = other RNA
cttgtccagctcctgccctggttttagagctagaaatagcaagttaaaataaggctagtccgttatcaacttgaaaaagtggcaccgagtcggtgctcgtcaatgcctcagggcaggagc

SEQ ID NO: 211 - RNA, 120 nt, synthetic construct
  source 1..120: mol_type = other RNA
cttgtccagctcctgccctggttttagagctagaaatagcaagttaaaataaggctagtccgttatcaacttgaaaaagtggcaccgagtcggtgctcaatgcctcagggcaggagctgg

SEQ ID NO: 212 - RNA, 120 nt, synthetic construct
  source 1..120: mol_type = other RNA
cttgtccagctcctgccctggttttagagctagaaatagcaagttaaaataaggctagtccgttatcaacttgaaaaagtggcaccgagtcggtgctcaatggctcagggcaggagctgg

SEQ ID NO: 213 - RNA, 120 nt, synthetic construct
  source 1..120: mol_type = other RNA
cttgtccagctcctgccctggttttagagctagaaatagcaagttaaaataaggctagtccgttatcaacttgaaaaagtggcaccgagtcggtgcgtcaatggctcagggcaggagctg

SEQ ID NO: 214 - RNA, 120 nt, synthetic construct
  source 1..120: mol_type = other RNA
cttgtccagctcctgccctggttttagagctagaaatagcaagttaaaataaggctagtccgttatcaacttgaaaaagtggcaccgagtcggtgctcgtcaatggctcagggcaggagc

SEQ ID NO: 215 - RNA, 120 nt, synthetic construct
  source 1..120: mol_type = other RNA
cttgtccagctcctgccctggttttagagctagaaatagcaagttaaaataaggctagtccgttatcaacttgaaaaagtggcaccgagtcggtgcttcaatgcctcagggcaggagctg

SEQ ID NO: 216 - RNA, 120 nt, synthetic construct
  source 1..120: mol_type = other RNA
cttgtccagctcctgccctggttttagagctagaaatagcaagttaaaataaggctagtccgttatcaacttgaaaaagtggcaccgagtcggtgctcaatgcgtcagggcaggagctgg

SEQ ID NO: 217 - RNA, 120 nt, synthetic construct
  source 1..120: mol_type = other RNA
cttgtccagctcctgccctggttttagagctagaaatagcaagttaaaataaggctagtccgttatcaacttgaaaaagtggcaccgagtcggtgcgtcaatgcgtcagggcaggagctg

SEQ ID NO: 218 - RNA, 120 nt, synthetic construct
  source 1..120: mol_type = other RNA
cttgtccagctcctgccctggttttagagctagaaatagcaagttaaaataaggctagtccgttatcaacttgaaaaagtggcaccgagtcggtgctcgtcaatgcgtcagggcaggagc

SEQ ID NO: 219 - RNA, 121 nt, synthetic construct
  source 1..121: mol_type = other RNA
cttgtccagctcctgccctggttttagagctagaaatagcaagttaaaataaggctagtccgttatcaacttgaaaaagtggcaccgagtcggtgctcgtcaatgcctcagggcaggagct

SEQ ID NO: 220 - RNA, 121 nt, synthetic construct
  source 1..121: mol_type = other RNA
cttgtccagctcctgccctggttttagagctagaaatagcaagttaaaataaggctagtccgttatcaacttgaaaaagtggcaccgagtcggtgcgtcaatgcctcagggcaggagctgg

SEQ ID NO: 221 - RNA, 121 nt, synthetic construct
  source 1..121: mol_type = other RNA
cttgtccagctcctgccctggttttagagctagaaatagcaagttaaaataaggctagtccgttatcaacttgaaaaagtggcaccgagtcggtgcgctcgtcaatgcctcagggcaggag

SEQ ID NO: 222 - RNA, 121 nt, synthetic construct
  source 1..121: mol_type = other RNA
cttgtccagctcctgccctggttttagagctagaaatagcaagttaaaataaggctagtccgttatcaacttgaaaaagtggcaccgagtcggtgctcaatgcctcagggcaggagctgga

SEQ ID NO: 223 - RNA, 121 nt, synthetic construct
  source 1..121: mol_type = other RNA
cttgtccagctcctgccctggttttagagctagaaatagcaagttaaaataaggctagtccgttatcaacttgaaaaagtggcaccgagtcggtgctcaatggctcagggcaggagctgga

SEQ ID NO: 224 - RNA, 121 nt, synthetic construct
  source 1..121: mol_type = other RNA
cttgtccagctcctgccctggttttagagctagaaatagcaagttaaaataaggctagtccgttatcaacttgaaaaagtggcaccgagtcggtgcgtcaatggctcagggcaggagctgg

SEQ ID NO: 225 - RNA, 121 nt, synthetic construct
  source 1..121: mol_type = other RNA
cttgtccagctcctgccctggttttagagctagaaatagcaagttaaaataaggctagtccgttatcaacttgaaaaagtggcaccgagtcggtgctcgtcaatggctcagggcaggagct

SEQ ID NO: 226 - RNA, 121 nt, synthetic construct
  source 1..121: mol_type = other RNA
cttgtccagctcctgccctggttttagagctagaaatagcaagttaaaataaggctagtccgttatcaacttgaaaaagtggcaccgagtcggtgcttcaatggctcagggcaggagctgg

SEQ ID NO: 227 - RNA, 121 nt, synthetic construct
  source 1..121: mol_type = other RNA
cttgtccagctcctgccctggttttagagctagaaatagcaagttaaaataaggctagtccgttatcaacttgaaaaagtggcaccgagtcggtgctcaatgcgtcagggcaggagctgga

SEQ ID NO: 228 - RNA, 121 nt, synthetic construct
  source 1..121: mol_type = other RNA
cttgtccagctcctgccctggttttagagctagaaatagcaagttaaaataaggctagtccgttatcaacttgaaaaagtggcaccgagtcggtgcgtcaatgcgtcagggcaggagctgg

SEQ ID NO: 229 - RNA, 121 nt, synthetic construct
  source 1..121: mol_type = other RNA
cttgtccagctcctgccctggttttagagctagaaatagcaagttaaaataaggctagtccgttatcaacttgaaaaagtggcaccgagtcggtgctcgtcaatgcgtcagggcaggagct

SEQ ID NO: 230 - RNA, 122 nt, synthetic construct
  source 1..122: mol_type = other RNA
cttgtccagctcctgccctggttttagagctagaaatagcaagttaaaataaggctagtccgttatcaacttgaaaaagtggcaccgagtcggtgctgctcgtcaatgcctcagggcaggag

SEQ ID NO: 231 - RNA, 122 nt, synthetic construct
  source 1..122: mol_type = other RNA
cttgtccagctcctgccctggttttagagctagaaatagcaagttaaaataaggctagtccgttatcaacttgaaaaagtggcaccgagtcggtgcgtcaatgcctcagggcaggagctgga

SEQ ID NO: 232 - RNA, 122 nt, synthetic construct
  source 1..122: mol_type = other RNA
cttgtccagctcctgccctggttttagagctagaaatagcaagttaaaataaggctagtccgttatcaacttgaaaaagtggcaccgagtcggtgctcgtcaatgcctcagggcaggagctg

SEQ ID NO: 233 - RNA, 122 nt, synthetic construct
  source 1..122: mol_type = other RNA
cttgtccagctcctgccctggttttagagctagaaatagcaagttaaaataaggctagtccgttatcaacttgaaaaagtggcaccgagtcggtgcgctcgtcaatgcctcagggcaggagc

SEQ ID NO: 234 - RNA, 122 nt, synthetic construct
  source 1..122: mol_type = other RNA
cttgtccagctcctgccctggttttagagctagaaatagcaagttaaaataaggctagtccgttatcaacttgaaaaagtggcaccgagtcggtgctcaatgcctcagggcaggagctggac

SEQ ID NO: 235 - RNA, 122 nt, synthetic construct
  source 1..122: mol_type = other RNA
cttgtccagctcctgccctggttttagagctagaaatagcaagttaaaataaggctagtccgttatcaacttgaaaaagtggcaccgagtcggtgctcaatggctcagggcaggagctggac

SEQ ID NO: 236 - RNA, 122 nt, synthetic construct
  source 1..122: mol_type = other RNA
cttgtccagctcctgccctggttttagagctagaaatagcaagttaaaataaggctagtccgttatcaacttgaaaaagtggcaccgagtcggtgcgtcaatggctcagggcaggagctgga

SEQ ID NO: 237 - RNA, 122 nt, synthetic construct
  source 1..122: mol_type = other RNA
cttgtccagctcctgccctggttttagagctagaaatagcaagttaaaataaggctagtccgttatcaacttgaaaaagtggcaccgagtcggtgctcgtcaatggctcagggcaggagctg

SEQ ID NO: 238 - RNA, 122 nt, synthetic construct
  source 1..122: mol_type = other RNA
cttgtccagctcctgccctggttttagagctagaaatagcaagttaaaataaggctagtccgttatcaacttgaaaaagtggcaccgagtcggtgcgctcgtcaatggctcagggcaggagc

SEQ ID NO: 239 - RNA, 122 nt, synthetic construct
  source 1..122: mol_type = other RNA
cttgtccagctcctgccctggttttagagctagaaatagcaagttaaaataaggctagtccgttatcaacttgaaaaagtggcaccgagtcggtgcttcaatggctcagggcaggagctgga

SEQ ID NO: 240 - RNA, 122 nt, synthetic construct
  source 1..122: mol_type = other RNA
cttgtccagctcctgccctggttttagagctagaaatagcaagttaaaataaggctagtccgttatcaacttgaaaaagtggcaccgagtcggtgcttcaatgcctcagggcaggagctgga

SEQ ID NO: 241 - RNA, 122 nt, synthetic construct
  source 1..122: mol_type = other RNA
cttgtccagctcctgccctggttttagagctagaaatagcaagttaaaataaggctagtccgttatcaacttgaaaaagtggcaccgagtcggtgctcaatgcgtcagggcaggagctggac

SEQ ID NO: 242 - RNA, 122 nt, synthetic construct
  source 1..122: mol_type = other RNA
cttgtccagctcctgccctggttttagagctagaaatagcaagttaaaataaggctagtccgttatcaacttgaaaaagtggcaccgagtcggtgcgtcaatgcgtcagggcaggagctgga

SEQ ID NO: 243 - RNA, 122 nt, synthetic construct
  source 1..122: mol_type = other RNA
cttgtccagctcctgccctggttttagagctagaaatagcaagttaaaataaggctagtccgttatcaacttgaaaaagtggcaccgagtcggtgctcgtcaatgcgtcagggcaggagctg

SEQ ID NO: 244 - RNA, 122 nt, synthetic construct
  source 1..122: mol_type = other RNA
cttgtccagctcctgccctggttttagagctagaaatagcaagttaaaataaggctagtccgttatcaacttgaaaaagtggcaccgagtcggtgcgctcgtcaatgcgtcagggcaggagc

SEQ ID NO: 245 - RNA, 123 nt, synthetic construct
  source 1..123: mol_type = other RNA
cttgtccagctcctgccctggttttagagctagaaatagcaagttaaaataaggctagtccgttatcaacttgaaaaagtggcaccgagtcggtgctcgtcaatgcctcagggcaggagctgg

SEQ ID NO: 246 - RNA, 123 nt, synthetic construct
  source 1..123: mol_type = other RNA
cttgtccagctcctgccctggttttagagctagaaatagcaagttaaaataaggctagtccgttatcaacttgaaaaagtggcaccgagtcggtgcgtcaatgcctcagggcaggagctggac

SEQ ID NO: 247 - RNA, 123 nt, synthetic construct
  source 1..123: mol_type = other RNA
cttgtccagctcctgccctggttttagagctagaaatagcaagttaaaataaggctagtccgttatcaacttgaaaaagtggcaccgagtcggtgcgctcgtcaatgcctcagggcaggagct

SEQ ID NO: 248 - RNA, 123 nt, synthetic construct
  source 1..123: mol_type = other RNA
cttgtccagctcctgccctggttttagagctagaaatagcaagttaaaataaggctagtccgttatcaacttgaaaaagtggcaccgagtcggtgctgctcgtcaatgcctcagggcaggagc

SEQ ID NO: 249 - RNA, 123 nt, synthetic construct
  source 1..123: mol_type = other RNA
cttgtccagctcctgccctggttttagagctagaaatagcaagttaaaataaggctagtccgttatcaacttgaaaaagtggcaccgagtcggtgctcgtcaatgcgtcagggcaggagctgg

SEQ ID NO: 250 - RNA, 123 nt, synthetic construct
  source 1..123: mol_type = other RNA
cttgtccagctcctgccctggttttagagctagaaatagcaagttaaaataaggctagtccgttatcaacttgaaaaagtggcaccgagtcggtgctcgtcaatgcatcagggcaggagctgg

SEQ ID NO: 251 - RNA, 123 nt, synthetic construct
  source 1..123: mol_type = other RNA
cttgtccagctcctgccctggttttagagctagaaatagcaagttaaaataaggctagtccgttatcaacttgaaaaagtggcaccgagtcggtgctcgtcaatgcttcagggcaggagctgg

SEQ ID NO: 252 - RNA, 123 nt, synthetic construct
  source 1..123: mol_type = other RNA
cttgtccagctcctgccctggttttagagctagaaatagcaagttaaaataaggctagtccgttatcaacttgaaaaagtggcaccgagtcggtgctcaatgcctcagggcaggagctggaca

SEQ ID NO: 253 - RNA, 123 nt, synthetic construct
  source 1..123: mol_type = other RNA
cttgtccagctcctgccctggttttagagctagaaatagcaagttaaaataaggctagtccgttatcaacttgaaaaagtggcaccgagtcggtgctcaatggctcagggcaggagctggaca

SEQ ID NO: 254 - RNA, 123 nt, synthetic construct
  source 1..123: mol_type = other RNA
cttgtccagctcctgccctggttttagagctagaaatagcaagttaaaataaggctagtccgttatcaacttgaaaaagtggcaccgagtcggtgcgtcaatggctcagggcaggagctggac

SEQ ID NO: 255 - RNA, 123 nt, synthetic construct
  source 1..123: mol_type = other RNA
cttgtccagctcctgccctggttttagagctagaaatagcaagttaaaataaggctagtccgttatcaacttgaaaaagtggcaccgagtcggtgctcgtcaatggctcagggcaggagctgg

SEQ ID NO: 256 - RNA, 123 nt, synthetic construct
  source 1..123: mol_type = other RNA
cttgtccagctcctgccctggttttagagctagaaatagcaagttaaaataaggctagtccgttatcaacttgaaaaagtggcaccgagtcggtgcgctcgtcaatggctcagggcaggagct

SEQ ID NO: 257 - RNA, 123 nt, synthetic construct
  source 1..123: mol_type = other RNA
cttgtccagctcctgccctggttttagagctagaaatagcaagttaaaataaggctagtccgttatcaacttgaaaaagtggcaccgagtcggtgctgctcgtcaatggctcagggcaggagc

SEQ ID NO: 258 - RNA, 123 nt, synthetic construct
  source 1..123: mol_type = other RNA
cttgtccagctcctgccctggttttagagctagaaatagcaagttaaaataaggctagtccgttatcaacttgaaaaagtggcaccgagtcggtgctctcgtcaatggctcagggcaggagct

SEQ ID NO: 259 - RNA, 123 nt, synthetic construct
  source 1..123: mol_type = other RNA
cttgtccagctcctgccctggttttagagctagaaatagcaagttaaaataaggctagtccgttatcaacttgaaaaagtggcaccgagtcggtgcttcaatggctcagggcaggagctggac

SEQ ID NO: 260 - RNA, 123 nt, synthetic construct
  source 1..123: mol_type = other RNA
cttgtccagctcctgccctggttttagagctagaaatagcaagttaaaataaggctagtccgttatcaacttgaaaaagtggcaccgagtcggtgctcaatgcgtcagggcaggagctggaca

SEQ ID NO: 261 - RNA, 123 nt, synthetic construct
  source 1..123: mol_type = other RNA
cttgtccagctcctgccctggttttagagctagaaatagcaagttaaaataaggctagtccgttatcaacttgaaaaagtggcaccgagtcggtgcgtcaatgcgtcagggcaggagctggac

SEQ ID NO: 262 - RNA, 123 nt, synthetic construct
  source 1..123: mol_type = other RNA
cttgtccagctcctgccctggttttagagctagaaatagcaagttaaaataaggctagtccgttatcaacttgaaaaagtggcaccgagtcggtgcgctcgtcaatgcgtcagggcaggagct

SEQ ID NO: 263 - RNA, 123 nt, synthetic construct
  source 1..123: mol_type = other RNA
cttgtccagctcctgccctggttttagagctagaaatagcaagttaaaataaggctagtccgttatcaacttgaaaaagtggcaccgagtcggtgctgctcgtcaatgcgtcagggcaggagc

SEQ ID NO: 264 - RNA, 124 nt, synthetic construct
  source 1..124: mol_type = other RNA
cttgtccagctcctgccctggttttagagctagaaatagcaagttaaaataaggctagtccgttatcaacttgaaaaagtggcaccgagtcggtgctgctcgtcaatgcctcagggcaggagct

SEQ ID NO: 265 - RNA, 124 nt, synthetic construct
  source 1..124: mol_type = other RNA
cttgtccagctcctgccctggttttagagctagaaatagcaagttaaaataaggctagtccgttatcaacttgaaaaagtggcaccgagtcggtgcgtcaatgcctcagggcaggagctggaca

SEQ ID NO: 266 - RNA, 124 nt, synthetic construct
  source 1..124: mol_type = other RNA
cttgtccagctcctgccctggttttagagctagaaatagcaagttaaaataaggctagtccgttatcaacttgaaaaagtggcaccgagtcggtgctcgtcaatgcctcagggcaggagctgga

SEQ ID NO: 267 - RNA, 124 nt, synthetic construct
  source 1..124: mol_type = other RNA
cttgtccagctcctgccctggttttagagctagaaatagcaagttaaaataaggctagtccgttatcaacttgaaaaagtggcaccgagtcggtgcgctcgtcaatgcctcagggcaggagctg

SEQ ID NO: 268 - RNA, 124 nt, synthetic construct
  source 1..124: mol_type = other RNA
cttgtccagctcctgccctggttttagagctagaaatagcaagttaaaataaggctagtccgttatcaacttgaaaaagtggcaccgagtcggtgctctgctcgtcaatgcctcagggcaggag

SEQ ID NO: 269 - RNA, 124 nt, synthetic construct
  source 1..124: mol_type = other RNA
cttgtccagctcctgccctggttttagagctagaaatagcaagttaaaataaggctagtccgttatcaacttgaaaaagtggcaccgagtcggtgcgtcaatggctcagggcaggagctggaca

SEQ ID NO: 270 - RNA, 124 nt, synthetic construct
  source 1..124: mol_type = other RNA
cttgtccagctcctgccctggttttagagctagaaatagcaagttaaaataaggctagtccgttatcaacttgaaaaagtggcaccgagtcggtgctcgtcaatggctcagggcaggagctgga

SEQ ID NO: 271 - RNA, 124 nt, synthetic construct
  source 1..124: mol_type = other RNA
cttgtccagctcctgccctggttttagagctagaaatagcaagttaaaataaggctagtccgttatcaacttgaaaaagtggcaccgagtcggtgcgctcgtcaatggctcagggcaggagctg

SEQ ID NO: 272 - RNA, 124 nt, synthetic construct
  source 1..124: mol_type = other RNA
cttgtccagctcctgccctggttttagagctagaaatagcaagttaaaataaggctagtccgttatcaacttgaaaaagtggcaccgagtcggtgctgctcgtcaatggctcagggcaggagct

SEQ ID NO: 273 - RNA, 124 nt, synthetic construct
  source 1..124: mol_type = other RNA
cttgtccagctcctgccctggttttagagctagaaatagcaagttaaaataaggctagtccgttatcaacttgaaaaagtggcaccgagtcggtgctctcgtcaatggctcagggcaggagctg

SEQ ID NO: 274 - RNA, 124 nt, synthetic construct
  source 1..124: mol_type = other RNA
cttgtccagctcctgccctggttttagagctagaaatagcaagttaaaataaggctagtccgttatcaacttgaaaaagtggcaccgagtcggtgcttcaatggctcagggcaggagctggaca

SEQ ID NO: 275 - RNA, 124 nt, synthetic construct
  source 1..124: mol_type = other RNA
cttgtccagctcctgccctggttttagagctagaaatagcaagttaaaataaggctagtccgttatcaacttgaaaaagtggcaccgagtcggtgcgtcaatgcgtcagggcaggagctggaca

SEQ ID NO: 276 - RNA, 124 nt, synthetic construct
  source 1..124: mol_type = other RNA
cttgtccagctcctgccctggttttagagctagaaatagcaagttaaaataaggctagtccgttatcaacttgaaaaagtggcaccgagtcggtgctcgtcaatgcgtcagggcaggagctgga

SEQ ID NO: 277 - RNA, 124 nt, synthetic construct
  source 1..124: mol_type = other RNA
cttgtccagctcctgccctggttttagagctagaaatagcaagttaaaataaggctagtccgttatcaacttgaaaaagtggcaccgagtcggtgcgctcgtcaatgcgtcagggcaggagctg

SEQ ID NO: 278 - RNA, 124 nt, synthetic construct
  source 1..124: mol_type = other RNA
cttgtccagctcctgccctggttttagagctagaaatagcaagttaaaataaggctagtccgttatcaacttgaaaaagtggcaccgagtcggtgctgctcgtcaatgcgtcagggcaggagct

SEQ ID NO: 279 - RNA, 125 nt, synthetic construct
  source 1..125: mol_type = other RNA
cttgtccagctcctgccctggttttagagctagaaatagcaagttaaaataaggctagtccgttatcaacttgaaaaagtggcaccgagtcggtgctcgtcaatgcctcagggcaggagctggac

SEQ ID NO: 280 - RNA, 125 nt, synthetic construct
  source 1..125: mol_type = other RNA
cttgtccagctcctgccctggttttagagctagaaatagcaagttaaaataaggctagtccgttatcaacttgaaaaagtggcaccgagtcggtgcgctcgtcaatgcctcagggcaggagctgg

SEQ ID NO: 281 - RNA, 125 nt, synthetic construct
  source 1..125: mol_type = other RNA
cttgtccagctcctgccctggttttagagctagaaatagcaagttaaaataaggctagtccgttatcaacttgaaaaagtggcaccgagtcggtgctgctcgtcaatgcctcagggcaggagctg

SEQ ID NO: 282 - RNA, 125 nt, synthetic construct
  source 1..125: mol_type = other RNA
cttgtccagctcctgccctggttttagagctagaaatagcaagttaaaataaggctagtccgttatcaacttgaaaaagtggcaccgagtcggtgctctgctcgtcaatgcctcagggcaggagc

SEQ ID NO: 283 - RNA, 125 nt, synthetic construct
  source 1..125: mol_type = other RNA
cttgtccagctcctgccctggttttagagctagaaatagcaagttaaaataaggctagtccgttatcaacttgaaaaagtggcaccgagtcggtgcgtcaatgcctcagggcaggagctggacaa

SEQ ID NO: 284 - RNA, 125 nt, synthetic construct
  source 1..125: mol_type = other RNA
cttgtccagctcctgccctggttttagagctagaaatagcaagttaaaataaggctagtccgttatcaacttgaaaaagtggcaccgagtcggtgctcgtcaatggctcagggcaggagctggac

SEQ ID NO: 285 - RNA, 125 nt, synthetic construct
  source 1..125: mol_type = other RNA
cttgtccagctcctgccctggttttagagctagaaatagcaagttaaaataaggctagtccgttatcaacttgaaaaagtggcaccgagtcggtgcgctcgtcaatggctcagggcaggagctgg

SEQ ID NO: 286 - RNA, 125 nt, synthetic construct
  source 1..125: mol_type = other RNA
cttgtccagctcctgccctggttttagagctagaaatagcaagttaaaataaggctagtccgttatcaacttgaaaaagtggcaccgagtcggtgctgctcgtcaatggctcagggcaggagctg

SEQ ID NO: 287 - RNA, 125 nt, synthetic construct
  source 1..125: mol_type = other RNA
cttgtccagctcctgccctggttttagagctagaaatagcaagttaaaataaggctagtccgttatcaacttgaaaaagtggcaccgagtcggtgctctcgtcaatggctcagggcaggagctgg

SEQ ID NO: 288 - RNA, 125 nt, synthetic construct
  source 1..125: mol_type = other RNA
cttgtccagctcctgccctggttttagagctagaaatagcaagttaaaataaggctagtccgttatcaacttgaaaaagtggcaccgagtcggtgctcgtcaatgcgtcagggcaggagctggac

SEQ ID NO: 289 - RNA, 125 nt, synthetic construct
  source 1..125: mol_type = other RNA
cttgtccagctcctgccctggttttagagctagaaatagcaagttaaaataaggctagtccgttatcaacttgaaaaagtggcaccgagtcggtgcgctcgtcaatgcgtcagggcaggagctgg

SEQ ID NO: 290 - RNA, 125 nt, synthetic construct
  source 1..125: mol_type = other RNA
cttgtccagctcctgccctggttttagagctagaaatagcaagttaaaataaggctagtccgttatcaacttgaaaaagtggcaccgagtcggtgctgctcgtcaatgcgtcagggcaggagctg

SEQ ID NO: 291 - RNA, 126 nt, synthetic construct
  source 1..126: mol_type = other RNA
cttgtccagctcctgccctggttttagagctagaaatagcaagttaaaataaggctagtccgttatcaacttgaaaaagtggcaccgagtcggtgcgctctgctcgtcaatgcctcagggcaggag

SEQ ID NO: 292 - RNA, 126 nt, synthetic construct
  source 1..126: mol_type = other RNA
cttgtccagctcctgccctggttttagagctagaaatagcaagttaaaataaggctagtccgttatcaacttgaaaaagtggcaccgagtcggtgctgctcgtcaatgcctcagggcaggagctgg

SEQ ID NO: 293 - RNA, 126 nt, synthetic construct
  source 1..126: mol_type = other RNA
cttgtccagctcctgccctggttttagagctagaaatagcaagttaaaataaggctagtccgttatcaacttgaaaaagtggcaccgagtcggtgctcgtcaatgcctcagggcaggagctggaca

SEQ ID NO: 294 - RNA, 126 nt, synthetic construct
  source 1..126: mol_type = other RNA
cttgtccagctcctgccctggttttagagctagaaatagcaagttaaaataaggctagtccgttatcaacttgaaaaagtggcaccgagtcggtgcgctcgtcaatgcctcagggcaggagctgga

SEQ ID NO: 295 - RNA, 126 nt, synthetic construct
  source 1..126: mol_type = other RNA
cttgtccagctcctgccctggttttagagctagaaatagcaagttaaaataaggctagtccgttatcaacttgaaaaagtggcaccgagtcggtgctctgctcgtcaatgcctcagggcaggagct

SEQ ID NO: 296 - RNA, 126 nt, synthetic construct
  source 1..126: mol_type = other RNA
cttgtccagctcctgccctggttttagagctagaaatagcaagttaaaataaggctagtccgttatcaacttgaaaaagtggcaccgagtcggtgctcgtcaatggctcagggcaggagctggaca

SEQ ID NO: 297 - RNA, 126 nt, synthetic construct
  source 1..126: mol_type = other RNA
cttgtccagctcctgccctggttttagagctagaaatagcaagttaaaataaggctagtccgttatcaacttgaaaaagtggcaccgagtcggtgcgctcgtcaatggctcagggcaggagctgga

SEQ ID NO: 298 - RNA, 126 nt, synthetic construct
  source 1..126: mol_type = other RNA
cttgtccagctcctgccctggttttagagctagaaatagcaagttaaaataaggctagtccgttatcaacttgaaaaagtggcaccgagtcggtgctgctcgtcaatggctcagggcaggagctgg

SEQ ID NO: 299 - RNA, 126 nt, synthetic construct
  source 1..126: mol_type = other RNA
cttgtccagctcctgccctggttttagagctagaaatagcaagttaaaataaggctagtccgttatcaacttgaaaaagtggcaccgagtcggtgctctcgtcaatggctcagggcaggagctgga

SEQ ID NO: 300 - RNA, 126 nt, synthetic construct
  source 1..126: mol_type = other RNA
cttgtccagctcctgccctggttttagagctagaaatagcaagttaaaataaggctagtccgttatcaacttgaaaaagtggcaccgagtcggtgctcgtcaatgcgtcagggcaggagctggaca

SEQ ID NO: 301 - RNA, 126 nt, synthetic construct
  source 1..126: mol_type = other RNA
cttgtccagctcctgccctggttttagagctagaaatagcaagttaaaataaggctagtccgttatcaacttgaaaaagtggcaccgagtcggtgcgctcgtcaatgcgtcagggcaggagctgga

SEQ ID NO: 302 - RNA, 126 nt, synthetic construct
  source 1..126: mol_type = other RNA
cttgtccagctcctgccctggttttagagctagaaatagcaagttaaaataaggctagtccgttatcaacttgaaaaagtggcaccgagtcggtgctgctcgtcaatgcgtcagggcaggagctgg

SEQ ID NO: 303 - RNA, 127 nt, synthetic construct
  source 1..127: mol_type = other RNA
cttgtccagctcctgccctggttttagagctagaaatagcaagttaaaataaggctagtccgttatcaacttgaaaaagtggcaccgagtcggtgcgctcgtcaatgcctcagggcaggagctggac

SEQ ID NO: 304 - RNA, 127 nt, synthetic construct
  source 1..127: mol_type = other RNA
cttgtccagctcctgccctggttttagagctagaaatagcaagttaaaataaggctagtccgttatcaacttgaaaaagtggcaccgagtcggtgctgctcgtcaatgcctcagggcaggagctgga

SEQ ID NO: 305 - RNA, 127 nt, synthetic construct
  source 1..127: mol_type = other RNA
cttgtccagctcctgccctggttttagagctagaaatagcaagttaaaataaggctagtccgttatcaacttgaaaaagtggcaccgagtcggtgctctgctcgtcaatgcctcagggcaggagctg

SEQ ID NO: 306 - RNA, 127 nt, synthetic construct
  source 1..127: mol_type = other RNA
cttgtccagctcctgccctggttttagagctagaaatagcaagttaaaataaggctagtccgttatcaacttgaaaaagtggcaccgagtcggtgcgctctgctcgtcaatgcctcagggcaggagc

SEQ ID NO: 307 - RNA, 127 nt, synthetic construct
  source 1..127: mol_type = other RNA
cttgtccagctcctgccctggttttagagctagaaatagcaagttaaaataaggctagtccgttatcaacttgaaaaagtggcaccgagtcggtgctcgtcaatgcctcagggcaggagctggacaa

SEQ ID NO: 308 - RNA, 127 nt, synthetic construct
  source 1..127: mol_type = other RNA
cttgtccagctcctgccctggttttagagctagaaatagcaagttaaaataaggctagtccgttatcaacttgaaaaagtggcaccgagtcggtgcgctcgtcaatggctcagggcaggagctggac

SEQ ID NO: 309 - RNA, 127 nt, synthetic construct
  source 1..127: mol_type = other RNA
cttgtccagctcctgccctggttttagagctagaaatagcaagttaaaataaggctagtccgttatcaacttgaaaaagtggcaccgagtcggtgctgctcgtcaatggctcagggcaggagctgga

SEQ ID NO: 310 - RNA, 127 nt, synthetic construct
  source 1..127: mol_type = other RNA
cttgtccagctcctgccctggttttagagctagaaatagcaagttaaaataaggctagtccgttatcaacttgaaaaagtggcaccgagtcggtgctctcgtcaatggctcagggcaggagctggac

SEQ ID NO: 311 - RNA, 127 nt, synthetic construct
  source 1..127: mol_type = other RNA
cttgtccagctcctgccctggttttagagctagaaatagcaagttaaaataaggctagtccgttatcaacttgaaaaagtggcaccgagtcggtgcgctcgtcaatgcgtcagggcaggagctggac

SEQ ID NO: 312 - RNA, 127 nt, synthetic construct
  source 1..127: mol_type = other RNA
cttgtccagctcctgccctggttttagagctagaaatagcaagttaaaataaggctagtccgttatcaacttgaaaaagtggcaccgagtcggtgctgctcgtcaatgcgtcagggcaggagctgga

SEQ ID NO: 313 - RNA, 128 nt, synthetic construct
  source 1..128: mol_type = other RNA
cttgtccagctcctgccctggttttagagctagaaatagcaagttaaaataaggctagtccgttatcaacttgaaaaagtggcaccgagtcggtgcgagctctgctcgtcaatgcctcagggcaggag

SEQ ID NO: 314 - RNA, 128 nt, synthetic construct
  source 1..128: mol_type = other RNA
cttgtccagctcctgccctggttttagagctagaaatagcaagttaaaataaggctagtccgttatcaacttgaaaaagtggcaccgagtcggtgcgctctgctcgtcaatgcctcagggcaggagct

SEQ ID NO: 315 - RNA, 128 nt, synthetic construct
  source 1..128: mol_type = other RNA
cttgtccagctcctgccctggttttagagctagaaatagcaagttaaaataaggctagtccgttatcaacttgaaaaagtggcaccgagtcggtgcgctcgtcaatgcctcagggcaggagctggaca

SEQ ID NO: 316 - RNA, 128 nt, synthetic construct
  source 1..128: mol_type = other RNA
cttgtccagctcctgccctggttttagagctagaaatagcaagttaaaataaggctagtccgttatcaacttgaaaaagtggcaccgagtcggtgctgctcgtcaatgcctcagggcaggagctggac

SEQ ID NO: 317 - RNA, 128 nt, synthetic construct
  source 1..128: mol_type = other RNA
cttgtccagctcctgccctggttttagagctagaaatagcaagttaaaataaggctagtccgttatcaacttgaaaaagtggcaccgagtcggtgctctgctcgtcaatgcctcagggcaggagctgg

SEQ ID NO: 318 - RNA, 128 nt, synthetic construct
  source 1..128: mol_type = other RNA
cttgtccagctcctgccctggttttagagctagaaatagcaagttaaaataaggctagtccgttatcaacttgaaaaagtggcaccgagtcggtgcgctcgtcaatggctcagggcaggagctggaca

SEQ ID NO: 319 - RNA, 128 nt, synthetic construct
  source 1..128: mol_type = other RNA
cttgtccagctcctgccctggttttagagctagaaatagcaagttaaaataaggctagtccgttatcaacttgaaaaagtggcaccgagtcggtgctgctcgtcaatggctcagggcaggagctggac

SEQ ID NO: 320 - RNA, 128 nt, synthetic construct
  source 1..128: mol_type = other RNA
cttgtccagctcctgccctggttttagagctagaaatagcaagttaaaataaggctagtccgttatcaacttgaaaaagtggcaccgagtcggtgctctcgtcaatggctcagggcaggagctggaca

SEQ ID NO: 321 - RNA, 128 nt, synthetic construct
  source 1..128: mol_type = other RNA
cttgtccagctcctgccctggttttagagctagaaatagcaagttaaaataaggctagtccgttatcaacttgaaaaagtggcaccgagtcggtgcgctcgtcaatgcgtcagggcaggagctggaca

SEQ ID NO: 322 - RNA, 128 nt, synthetic construct
  source 1..128: mol_type = other RNA
cttgtccagctcctgccctggttttagagctagaaatagcaagttaaaataaggctagtccgttatcaacttgaaaaagtggcaccgagtcggtgctgctcgtcaatgcgtcagggcaggagctggac

SEQ ID NO: 323 - RNA, 129 nt, synthetic construct
  source 1..129: mol_type = other RNA
cttgtccagctcctgccctggttttagagctagaaatagcaagttaaaataaggctagtccgttatcaacttgaaaaagtggcaccgagtcggtgcgctcgtcaatgcctcagggcaggagctggacaa

SEQ ID NO: 324 - RNA, 129 nt, synthetic construct
  source 1..129: mol_type = other RNA
cttgtccagctcctgccctggttttagagctagaaatagcaagttaaaataaggctagtccgttatcaacttgaaaaagtggcaccgagtcggtgctgctcgtcaatgcctcagggcaggagctggaca

SEQ ID NO: 325 - RNA, 129 nt, synthetic construct
  source 1..129: mol_type = other RNA
cttgtccagctcctgccctggttttagagctagaaatagcaagttaaaataaggctagtccgttatcaacttgaaaaagtggcaccgagtcggtgctctgctcgtcaatgcctcagggcaggagctgga

SEQ ID NO: 326 - RNA, 129 nt, synthetic construct
  source 1..129: mol_type = other RNA
cttgtccagctcctgccctggttttagagctagaaatagcaagttaaaataaggctagtccgttatcaacttgaaaaagtggcaccgagtcggtgcgctctgctcgtcaatgcctcagggcaggagctg

SEQ ID NO: 327 - RNA, 129 nt, synthetic construct
  source 1..129: mol_type = other RNA
cttgtccagctcctgccctggttttagagctagaaatagcaagttaaaataaggctagtccgttatcaacttgaaaaagtggcaccgagtcggtgcgagctctgctcgtcaatgcctcagggcaggagc

SEQ ID NO: 328 - RNA, 129 nt, synthetic construct
  source 1..129: mol_type = other RNA
cttgtccagctcctgccctggttttagagctagaaatagcaagttaaaataaggctagtccgttatcaacttgaaaaagtggcaccgagtcggtgctgctcgtcaatggctcagggcaggagctggaca

SEQ ID NO: 329 - RNA, 129 nt, synthetic construct
  source 1..129: mol_type = other RNA
cttgtccagctcctgccctggttttagagctagaaatagcaagttaaaataaggctagtccgttatcaacttgaaaaagtggcaccgagtcggtgctgctcgtcaatgcgtcagggcaggagctggaca

SEQ ID NO: 330 - RNA, 130 nt, synthetic construct
  source 1..130: mol_type = other RNA
cttgtccagctcctgccctggttttagagctagaaatagcaagttaaaataaggctagtccgttatcaacttgaaaaagtggcaccgagtcggtgcgggagctctgctcgtcaatgcctcagggcaggag

SEQ ID NO: 331 - RNA, 130 nt, synthetic construct
  source 1..130: mol_type = other RNA
cttgtccagctcctgccctggttttagagctagaaatagcaagttaaaataaggctagtccgttatcaacttgaaaaagtggcaccgagtcggtgcgagctctgctcgtcaatgcctcagggcaggagct

SEQ ID NO: 332 - RNA, 130 nt, synthetic construct
  source 1..130: mol_type = other RNA
cttgtccagctcctgccctggttttagagctagaaatagcaagttaaaataaggctagtccgttatcaacttgaaaaagtggcaccgagtcggtgcgctctgctcgtcaatgcctcagggcaggagctgg

SEQ ID NO: 333 - RNA, 130 nt, synthetic construct
  source 1..130: mol_type = other RNA
cttgtccagctcctgccctggttttagagctagaaatagcaagttaaaataaggctagtccgttatcaacttgaaaaagtggcaccgagtcggtgctctgctcgtcaatgcctcagggcaggagctggac

SEQ ID NO: 334 - RNA, 130 nt, synthetic construct
  source 1..130: mol_type = other RNA
cttgtccagctcctgccctggttttagagctagaaatagcaagttaaaataaggctagtccgttatcaacttgaaaaagtggcaccgagtcggtgctgctcgtcaatgcctcagggcaggagctggacaa

SEQ ID NO: 335 - RNA, 131 nt, synthetic construct
  source 1..131: mol_type = other RNA
cttgtccagctcctgccctggttttagagctagaaatagcaagttaaaataaggctagtccgttatcaacttgaaaaagtggcaccgagtcggtgctctgctcgtcaatgcctcagggcaggagctggaca

SEQ ID NO: 336 - RNA, 131 nt, synthetic construct
  source 1..131: mol_type = other RNA
cttgtccagctcctgccctggttttagagctagaaatagcaagttaaaataaggctagtccgttatcaacttgaaaaagtggcaccgagtcggtgcgctctgctcgtcaatgcctcagggcaggagctgga

SEQ ID NO: 337 - RNA, 131 nt, synthetic construct
  source 1..131: mol_type = other RNA
cttgtccagctcctgccctggttttagagctagaaatagcaagttaaaataaggctagtccgttatcaacttgaaaaagtggcaccgagtcggtgcgagctctgctcgtcaatgcctcagggcaggagctg

SEQ ID NO: 338 - RNA, 131 nt, synthetic construct
  source 1..131: mol_type = other RNA
cttgtccagctcctgccctggttttagagctagaaatagcaagttaaaataaggctagtccgttatcaacttgaaaaagtggcaccgagtcggtgcgggagctctgctcgtcaatgcctcagggcaggagc

SEQ ID NO: 339 - RNA, 132 nt, synthetic construct
  source 1..132: mol_type = other RNA
cttgtccagctcctgccctggttttagagctagaaatagcaagttaaaataaggctagtccgttatcaacttgaaaaagtggcaccgagtcggtgcgggagctctgctcgtcaatgcctcagggcaggagct

SEQ ID NO: 340 - RNA, 132 nt, synthetic construct
  source 1..132: mol_type = other RNA
cttgtccagctcctgccctggttttagagctagaaatagcaagttaaaataaggctagtccgttatcaacttgaaaaagtggcaccgagtcggtgcgagctctgctcgtcaatgcctcagggcaggagctgg

SEQ ID NO: 341 - RNA, 132 nt, synthetic construct
  source 1..132: mol_type = other RNA
cttgtccagctcctgccctggttttagagctagaaatagcaagttaaaataaggctagtccgttatcaacttgaaaaagtggcaccgagtcggtgcgctctgctcgtcaatgcctcagggcaggagctggac

SEQ ID NO: 342 - RNA, 132 nt, synthetic construct
  source 1..132: mol_type = other RNA
cttgtccagctcctgccctggttttagagctagaaatagcaagttaaaataaggctagtccgttatcaacttgaaaaagtggcaccgagtcggtgctctgctcgtcaatgcctcagggcaggagctggacaa

SEQ ID NO: 343 - RNA, 133 nt, synthetic construct
  source 1..133: mol_type = other RNA
cttgtccagctcctgccctggttttagagctagaaatagcaagttaaaataaggctagtccgttatcaacttgaaaaagtggcaccgagtcggtgcgctctgctcgtcaatgcctcagggcaggagctggaca

SEQ ID NO: 344 - RNA, 133 nt, synthetic construct
  source 1..133: mol_type = other RNA
cttgtccagctcctgccctggttttagagctagaaatagcaagttaaaataaggctagtccgttatcaacttgaaaaagtggcaccgagtcggtgcgagctctgctcgtcaatgcctcagggcaggagctgga

SEQ ID NO: 345 - RNA, 134 nt, synthetic construct
  source 1..134: mol_type = other RNA
cttgtccagctcctgccctggttttagagctagaaatagcaagttaaaataaggctagtccgttatcaacttgaaaaagtggcaccgagtcggtgcgggagctctgctcgtcaatgcctcagggcaggagctgg

SEQ ID NO: 346 - RNA, 134 nt, synthetic construct
  source 1..134: mol_type = other RNA
cttgtccagctcctgccctggttttagagctagaaatagcaagttaaaataaggctagtccgttatcaacttgaaaaagtggcaccgagtcggtgcgagctctgctcgtcaatgcctcagggcaggagctggac

SEQ ID NO: 347 - RNA, 134 nt, synthetic construct
  source 1..134: mol_type = other RNA
cttgtccagctcctgccctggttttagagctagaaatagcaagttaaaataaggctagtccgttatcaacttgaaaaagtggcaccgagtcggtgcgctctgctcgtcaatgcctcagggcaggagctggacaa

SEQ ID NO: 348 - RNA, 135 nt, synthetic construct
  source 1..135: mol_type = other RNA
cttgtccagctcctgccctggttttagagctagaaatagcaagttaaaataaggctagtccgttatcaacttgaaaaagtggcaccgagtcggtgcgagctctgctcgtcaatgcctcagggcaggagctggaca

SEQ ID NO: 349 - RNA, 136 nt, synthetic construct
  source 1..136: mol_type = other RNA
cttgtccagctcctgccctggttttagagctagaaatagcaagttaaaataaggctagtccgttatcaacttgaaaaagtggcaccgagtcggtgcgggagctctgctcgtcaatgcctcagggcaggagctggac

SEQ ID NO: 350 - RNA, 136 nt, synthetic construct
  source 1..136: mol_type = other RNA
cttgtccagctcctgccctggttttagagctagaaatagcaagttaaaataaggctagtccgttatcaacttgaaaaagtggcaccgagtcggtgcgagctctgctcgtcaatgcctcagggcaggagctggacaa

SEQ ID NO: 351 - RNA, 138 nt, synthetic construct
  source 1..138: mol_type = other RNA
cttgtccagctcctgccctggttttagagctagaaatagcaagttaaaataaggctagtccgttatcaacttgaaaaagtggcaccgagtcggtgcgggagctctgctcgtcaatgcctcagggcaggagctggacaa

SEQ ID NO: 352 - RNA, 147 nt, synthetic construct
  source 1..147: mol_type = other RNA
cttgtccagctcctgccctggttttagagctagaaatagcaagttaaaataaggctagtccgttatcaacttgaaaaagtggcaccgagtcggtgcgtcaatgcctcagggcaggagaacattgacgcgtctctacgtgggggcgcg

SEQ ID NO: 353 - RNA, 148 nt, synthetic construct
  source 1..148: mol_type = other RNA
cttgtccagctcctgccctggttttagagctagaaatagcaagttaaaataaggctagtccgttatcaacttgaaaaagtggcaccgagtcggtgcgtcaatgcctcagggcaggagcaacattgacgcgtctctacgtgggggcgcg

SEQ ID NO: 354 - RNA, 148 nt, synthetic construct
  source 1..148: mol_type = other RNA
cttgtccagctcctgccctggttttagagctagaaatagcaagttaaaataaggctagtccgttatcaacttgaaaaagtggcaccgagtcggtgctcaatgcctcagggcaggagctaacattgacgcgtctctacgtgggggcgcg

SEQ ID NO: 355 - RNA, 148 nt, synthetic construct
  source 1..148: mol_type = other RNA
cttgtccagctcctgccctggttttagagctagaaatagcaagttaaaataaggctagtccgttatcaacttgaaaaagtggcaccgagtcggtgctcaatggctcagggcaggagctaacattgacgcgtctctacgtgggggcgcg

SEQ ID NO: 356 - RNA, 148 nt, synthetic construct
  source 1..148: mol_type = other RNA
cttgtccagctcctgccctggttttagagctagaaatagcaagttaaaataaggctagtccgttatcaacttgaaaaagtggcaccgagtcggtgcgtcaatggctcagggcaggagcaacattgacgcgtctctacgtgggggcgcg

SEQ ID NO: 357 - RNA, 148 nt, synthetic construct
  source 1..148: mol_type = other RNA
cttgtccagctcctgccctggttttagagctagaaatagcaagttaaaataaggctagtccgttatcaacttgaaaaagtggcaccgagtcggtgctcaatgcgtcagggcaggagctaacattgacgcgtctctacgtgggggcgcg

SEQ ID NO: 358 - RNA, 148 nt, synthetic construct
  source 1..148: mol_type = other RNA
cttgtccagctcctgccctggttttagagctagaaatagcaagttaaaataaggctagtccgttatcaacttgaaaaagtggcaccgagtcggtgcgtcaatgcgtcagggcaggagcaacattgacgcgtctctacgtgggggcgcg

SEQ ID NO: 359 - RNA, 149 nt, synthetic construct
  source 1..149: mol_type = other RNA
cttgtccagctcctgccctggttttagagctagaaatagcaagttaaaataaggctagtccgttatcaacttgaaaaagtggcaccgagtcggtgctcgtcaatgcctcagggcaggagaacattgacgcgtctctacgtgggggcgcg

SEQ ID NO: 360 - RNA, 149 nt, synthetic construct
  source 1..149: mol_type = other RNA
cttgtccagctcctgccctggttttagagctagaaatagcaagttaaaataaggctagtccgttatcaacttgaaaaagtggcaccgagtcggtgcgtcaatgcctcagggcaggagctaacattgacgcgtctctacgtgggggcgcg

SEQ ID NO: 361 - RNA, 149 nt, synthetic construct
  source 1..149: mol_type = other RNA
cttgtccagctcctgccctggttttagagctagaaatagcaagttaaaataaggctagtccgttatcaacttgaaaaagtggcaccgagtcggtgctcaatgcctcagggcaggagctgaacattgacgcgtctctacgtgggggcgcg

SEQ ID NO: 362 - RNA, 149 nt, synthetic construct
  source 1..149: mol_type = other RNA
cttgtccagctcctgccctggttttagagctagaaatagcaagttaaaataaggctagtccgttatcaacttgaaaaagtggcaccgagtcggtgctcaatggctcagggcaggagctgaacattgacgcgtctctacgtgggggcgcg

SEQ ID NO: 363 - RNA, 149 nt, synthetic construct
  source 1..149: mol_type = other RNA
cttgtccagctcctgccctggttttagagctagaaatagcaagttaaaataaggctagtccgttatcaacttgaaaaagtggcaccgagtcggtgcgtcaatggctcagggcaggagctaacattgacgcgtctctacgtgggggcgcg

SEQ ID NO: 364 - RNA, 149 nt, synthetic construct
  source 1..149: mol_type = other RNA
cttgtccagctcctgccctggttttagagctagaaatagcaagttaaaataaggctagtccgttatcaacttgaaaaagtggcaccgagtcggtgctcaatgcgtcagggcaggagctgaacattgacgcgtctctacgtgggggcgcg

SEQ ID NO: 365 - RNA, 149 nt, synthetic construct
  source 1..149: mol_type = other RNA
cttgtccagctcctgccctggttttagagctagaaatagcaagttaaaataaggctagtccgttatcaacttgaaaaagtggcaccgagtcggtgcgtcaatgcgtcagggcaggagctaacattgacgcgtctctacgtgggggcgcg

SEQ ID NO: 366 - RNA, 150 nt, synthetic construct
  source 1..150: mol_type = other RNA
cttgtccagctcctgccctggttttagagctagaaatagcaagttaaaataaggctagtccgttatcaacttgaaaaagtggcaccgagtcggtgcgtcaatgcctcagggcaggagctgaacattgacgcgtctctacgtgggggcgcg

SEQ ID NO: 367 - RNA, 150 nt, synthetic construct
  source 1..150: mol_type = other RNA
cttgtccagctcctgccctggttttagagctagaaatagcaagttaaaataaggctagtccgttatcaacttgaaaaagtggcaccgagtcggtgctcgtcaatgcctcagggcaggagcaacattgacgcgtctctacgtgggggcgcg

SEQ ID NO: 368 - RNA, 150 nt, synthetic construct
  source 1..150: mol_type = other RNA
cttgtccagctcctgccctggttttagagctagaaatagcaagttaaaataaggctagtccgttatcaacttgaaaaagtggcaccgagtcggtgctcaatgcctcagggcaggagctggaacattgacgcgtctctacgtgggggcgcg

SEQ ID NO: 369 - RNA, 150 nt, synthetic construct
  source 1..150: mol_type = other RNA
cttgtccagctcctgccctggttttagagctagaaatagcaagttaaaataaggctagtccgttatcaacttgaaaaagtggcaccgagtcggtgctcaatggctcagggcaggagctggaacattgacgcgtctctacgtgggggcgcg

SEQ ID NO: 370 - RNA, 150 nt, synthetic construct
  source 1..150: mol_type = other RNA
cttgtccagctcctgccctggttttagagctagaaatagcaagttaaaataaggctagtccgttatcaacttgaaaaagtggcaccgagtcggtgcgtcaatggctcagggcaggagctgaacattgacgcgtctctacgtgggggcgcg

SEQ ID NO: 371 - RNA, 150 nt, synthetic construct
  source 1..150: mol_type = other RNA
cttgtccagctcctgccctggttttagagctagaaatagcaagttaaaataaggctagtccgttatcaacttgaaaaagtggcaccgagtcggtgctcgtcaatggctcagggcaggagcaacattgacgcgtctctacgtgggggcgcg

SEQ ID NO: 372 - RNA, 150 nt, synthetic construct
  source 1..150: mol_type = other RNA
cttgtccagctcctgccctggttttagagctagaaatagcaagttaaaataaggctagtccgttatcaacttgaaaaagtggcaccgagtcggtgcttcaatgcctcagggcaggagctgaacattgacgcgtctctacgtgggggcgcg

SEQ ID NO: 373 - RNA, 150 nt, synthetic construct
  source 1..150: mol_type = other RNA
cttgtccagctcctgccctggttttagagctagaaatagcaagttaaaataaggctagtccgttatcaacttgaaaaagtggcaccgagtcggtgctcaatgcgtcagggcaggagctggaacattgacgcgtctctacgtgggggcgcg

SEQ ID NO: 374 - RNA, 150 nt, synthetic construct
  source 1..150: mol_type = other RNA
cttgtccagctcctgccctggttttagagctagaaatagcaagttaaaataaggctagtccgttatcaacttgaaaaagtggcaccgagtcggtgcgtcaatgcgtcagggcaggagctgaacattgacgcgtctctacgtgggggcgcg

SEQ ID NO: 375 - RNA, 150 nt, synthetic construct
  source 1..150: mol_type = other RNA
cttgtccagctcctgccctggttttagagctagaaatagcaagttaaaataaggctagtccgttatcaacttgaaaaagtggcaccgagtcggtgctcgtcaatgcgtcagggcaggagcaacattgacgcgtctctacgtgggggcgcg

SEQ ID NO: 376 - RNA, 151 nt, synthetic construct
  source 1..151: mol_type = other RNA
cttgtccagctcctgccctggttttagagctagaaatagcaagttaaaataaggctagtccgttatcaacttgaaaaagtggcaccgagtcggtgctcgtcaatgcctcagggcaggagctaacattgacgcgtctctacgtgggggcgcg

SEQ ID NO: 377 - RNA, 151 nt, synthetic construct
  source 1..151: mol_type = other RNA
cttgtccagctcctgccctggttttagagctagaaatagcaagttaaaataaggctagtccgttatcaacttgaaaaagtggcaccgagtcggtgcgtcaatgcctcagggcaggagctggaacattgacgcgtctctacgtgggggcgcg

SEQ ID NO: 378 - RNA, 151 nt, synthetic construct
  source 1..151: mol_type = other RNA
cttgtccagctcctgccctggttttagagctagaaatagcaagttaaaataaggctagtccgttatcaacttgaaaaagtggcaccgagtcggtgcgctcgtcaatgcctcagggcaggagaacattgacgcgtctctacgtgggggcgcg

SEQ ID NO: 379 - RNA, 151 nt, synthetic construct
  source 1..151: mol_type = other RNA
cttgtccagctcctgccctggttttagagctagaaatagcaagttaaaataaggctagtccgttatcaacttgaaaaagtggcaccgagtcggtgctcaatgcctcagggcaggagctggaaacattgacgcgtctctacgtgggggcgcg

SEQ ID NO: 380 - RNA, 151 nt, synthetic construct
  source 1..151: mol_type = other RNA
cttgtccagctcctgccctggttttagagctagaaatagcaagttaaaataaggctagtccgttatcaacttgaaaaagtggcaccgagtcggtgctcaatggctcagggcaggagctggaaacattgacgcgtctctacgtgggggcgcg

SEQ ID NO: 381 - RNA, 151 nt, synthetic construct
  source 1..151: mol_type = other RNA
cttgtccagctcctgccctggttttagagctagaaatagcaagttaaaataaggctagtccgttatcaacttgaaaaagtggcaccgagtcggtgcgtcaatggctcagggcaggagctggaacattgacgcgtctctacgtgggggcgcg

SEQ ID NO: 382 - RNA, 151 nt, synthetic construct
  source 1..151: mol_type = other RNA
cttgtccagctcctgccctggttttagagctagaaatagcaagttaaaataaggctagtccgttatcaacttgaaaaagtggcaccgagtcggtgctcgtcaatggctcagggcaggagctaacattgacgcgtctctacgtgggggcgcg

SEQ ID NO: 383 - RNA, 151 nt, synthetic construct
  source 1..151: mol_type = other RNA
cttgtccagctcctgccctggttttagagctagaaatagcaagttaaaataaggctagtccgttatcaacttgaaaaagtggcaccgagtcggtgcttcaatggctcagggcaggagctggaacattgacgcgtctctacgtgggggcgcg

SEQ ID NO: 384 - RNA, 151 nt, synthetic construct
  source 1..151: mol_type = other RNA
cttgtccagctcctgccctggttttagagctagaaatagcaagttaaaataaggctagtccgttatcaacttgaaaaagtggcaccgagtcggtgctcaatgcgtcagggcaggagctggaaacattgacgcgtctctacgtgggggcgcg

SEQ ID NO: 385 - RNA, 151 nt, synthetic construct
  source 1..151: mol_type = other RNA
cttgtccagctcctgccctggttttagagctagaaatagcaagttaaaataaggctagtccgttatcaacttgaaaaagtggcaccgagtcggtgcgtcaatgcgtcagggcaggagctggaacattgacgcgtctctacgtgggggcgcg

SEQ ID NO: 386 - RNA, 151 nt, synthetic construct
  source 1..151: mol_type = other RNA
cttgtccagctcctgccctggttttagagctagaaatagcaagttaaaataaggctagtccgttatcaacttgaaaaagtggcaccgagtcggtgctcgtcaatgcgtcagggcaggagctaacattgacgcgtctctacgtgggggcgcg

SEQ ID NO: 387 - RNA, 152 nt, synthetic construct
  source 1..152: mol_type = other RNA
cttgtccagctcctgccctggttttagagctagaaatagcaagttaaaataaggctagtccgttatcaacttgaaaaagtggcaccgagtcggtgctgctcgtcaatgcctcagggcaggagaacattgacgcgtctctacgtgggggcgcg

SEQ ID NO: 388 - RNA, 152 nt, synthetic construct
  source 1..152: mol_type = other RNA
cttgtccagctcctgccctggttttagagctagaaatagcaagttaaaataaggctagtccgttatcaacttgaaaaagtggcaccgagtcggtgcgtcaatgcctcagggcaggagctggaaacattgacgcgtctctacgtgggggcgcg

SEQ ID NO: 389 - RNA, 152 nt, synthetic construct
  source 1..152: mol_type = other RNA
cttgtccagctcctgccctggttttagagctagaaatagcaagttaaaataaggctagtccgttatcaacttgaaaaagtggcaccgagtcggtgctcgtcaatgcctcagggcaggagctgaacattgacgcgtctctacgtgggggcgcg

SEQ ID NO: 390 - RNA, 152 nt, synthetic construct
  source 1..152: mol_type = other RNA
cttgtccagctcctgccctggttttagagctagaaatagcaagttaaaataaggctagtccgttatcaacttgaaaaagtggcaccgagtcggtgcgctcgtcaatgcctcagggcaggagcaacattgacgcgtctctacgtgggggcgcg

SEQ ID NO: 391 - RNA, 152 nt, synthetic construct
  source 1..152: mol_type = other RNA
cttgtccagctcctgccctggttttagagctagaaatagcaagttaaaataaggctagtccgttatcaacttgaaaaagtggcaccgagtcggtgctcaatgcctcagggcaggagctggacaacattgacgcgtctctacgtgggggcgcg

SEQ ID NO: 392 - RNA, 152 nt, synthetic construct
  source 1..152: mol_type = other RNA
cttgtccagctcctgccctggttttagagctagaaatagcaagttaaaataaggctagtccgttatcaacttgaaaaagtggcaccgagtcggtgctcaatggctcagggcaggagctggacaacattgacgcgtctctacgtgggggcgcg

SEQ ID NO: 393 - RNA, 152 nt, synthetic construct
  source 1..152: mol_type = other RNA
cttgtccagctcctgccctggttttagagctagaaatagcaagttaaaataaggctagtccgttatcaacttgaaaaagtggcaccgagtcggtgcgtcaatggctcagggcaggagctggaaacattgacgcgtctctacgtgggggcgcg

SEQ ID NO: 394 - RNA, 152 nt, synthetic construct
  source 1..152: mol_type = other RNA
cttgtccagctcctgccctggttttagagctagaaatagcaagttaaaataaggctagtccgttatcaacttgaaaaagtggcaccgagtcggtgctcgtcaatggctcagggcaggagctgaacattgacgcgtctctacgtgggggcgcg

SEQ ID NO: 395 - RNA, 152 nt, synthetic construct
  source 1..152: mol_type = other RNA
cttgtccagctcctgccctggttttagagctagaaatagcaagttaaaataaggctagtccgttatcaacttgaaaaagtggcaccgagtcggtgcgctcgtcaatggctcagggcaggagcaacattgacgcgtctctacgtgggggcgcg

SEQ ID NO: 396 - RNA, 152 nt, synthetic construct
  source 1..152: mol_type = other RNA
cttgtccagctcctgccctggttttagagctagaaatagcaagttaaaataaggctagtccgttatcaacttgaaaaagtggcaccgagtcggtgcttcaatggctcagggcaggagctggaaacattgacgcgtctctacgtgggggcgcg

SEQ ID NO: 397 - RNA, 152 nt, synthetic construct
  source 1..152: mol_type = other RNA
cttgtccagctcctgccctggttttagagctagaaatagcaagttaaaataaggctagtccgttatcaacttgaaaaagtggcaccgagtcggtgcttcaatgcctcagggcaggagctggaaacattgacgcgtctctacgtgggggcgcg

SEQ ID NO: 398 - RNA, 152 nt, synthetic construct
  source 1..152: mol_type = other RNA
cttgtccagctcctgccctggttttagagctagaaatagcaagttaaaataaggctagtccgttatcaacttgaaaaagtggcaccgagtcggtgctcaatgcgtcagggcaggagctggacaacattgacgcgtctctacgtgggggcgcg

SEQ ID NO: 399 - RNA, 152 nt, synthetic construct
  source 1..152: mol_type = other RNA
cttgtccagctcctgccctggttttagagctagaaatagcaagttaaaataaggctagtccgttatcaacttgaaaaagtggcaccgagtcggtgcgtcaatgcgtcagggcaggagctggaaacattgacgcgtctctacgtgggggcgcg

SEQ ID NO: 400 - RNA, 152 nt, synthetic construct
  source 1..152: mol_type = other RNA
cttgtccagctcctgccctggttttagagctagaaatagcaagttaaaataaggctagtccgttatcaacttgaaaaagtggcaccgagtcggtgctcgtcaatgcgtcagggcaggagctgaacattgacgcgtctctacgtgggggcgcg

SEQ ID NO: 401 - RNA, 152 nt, synthetic construct
  source 1..152: mol_type = other RNA
cttgtccagctcctgccctggttttagagctagaaatagcaagttaaaataaggctagtccgttatcaacttgaaaaagtggcaccgagtcggtgcgctcgtcaatgcgtcagggcaggagcaacattgacgcgtctctacgtgggggcgcg

SEQ ID NO: 402 - RNA, 153 nt, synthetic construct
  source 1..153: mol_type = other RNA
cttgtccagctcctgccctggttttagagctagaaatagcaagttaaaataaggctagtccgttatcaacttgaaaaagtggcaccgagtcggtgctcgtcaatgcctcagggcaggagctggaacattgacgcgtctctacgtgggggcgcg

SEQ ID NO: 403 - RNA, 153 nt, synthetic construct
  source 1..153: mol_type = other RNA
cttgtccagctcctgccctggttttagagctagaaatagcaagttaaaataaggctagtccgttatcaacttgaaaaagtggcaccgagtcggtgcgtcaatgcctcagggcaggagctggacaacattgacgcgtctctacgtgggggcgcg

SEQ ID NO: 404 - RNA, 153 nt, synthetic construct
  source 1..153: mol_type = other RNA
cttgtccagctcctgccctggttttagagctagaaatagcaagttaaaataaggctagtccgttatcaacttgaaaaagtggcaccgagtcggtgcgctcgtcaatgcctcagggcaggagctaacattgacgcgtctctacgtgggggcgcg

SEQ ID NO: 405 - RNA, 153 nt, synthetic construct
  source 1..153: mol_type = other RNA
cttgtccagctcctgccctggttttagagctagaaatagcaagttaaaataaggctagtccgttatcaacttgaaaaagtggcaccgagtcggtgctgctcgtcaatgcctcagggcaggagcaacattgacgcgtctctacgtgggggcgcg

SEQ ID NO: 406 - RNA, 153 nt, synthetic construct
  source 1..153: mol_type = other RNA
cttgtccagctcctgccctggttttagagctagaaatagcaagttaaaataaggctagtccgttatcaacttgaaaaagtggcaccgagtcggtgctcgtcaatgcgtcagggcaggagctggaacattgacgcgtctctacgtgggggcgcg

SEQ ID NO: 407 - RNA, 153 nt, synthetic construct
  source 1..153: mol_type = other RNA
cttgtccagctcctgccctggttttagagctagaaatagcaagttaaaataaggctagtccgttatcaacttgaaaaagtggcaccgagtcggtgctcgtcaatgcatcagggcaggagctggaacattgacgcgtctctacgtgggggcgcg

SEQ ID NO: 408 - RNA, 153 nt, synthetic construct
  source 1..153: mol_type = other RNA
cttgtccagctcctgccctggttttagagctagaaatagcaagttaaaataaggctagtccgttatcaacttgaaaaagtggcaccgagtcggtgctcgtcaatgcttcagggcaggagctggaacattgacgcgtctctacgtgggggcgcg

SEQ ID NO: 409 - RNA, 153 nt, synthetic construct
  source 1..153: mol_type = other RNA
cttgtccagctcctgccctggttttagagctagaaatagcaagttaaaataaggctagtccgttatcaacttgaaaaagtggcaccgagtcggtgctcaatgcctcagggcaggagctggacaaacattgacgcgtctctacgtgggggcgcg

SEQ ID NO: 410 - RNA, 153 nt, synthetic construct
  source 1..153: mol_type = other RNA
cttgtccagctcctgccctggttttagagctagaaatagcaagttaaaataaggctagtccgttatcaacttgaaaaagtggcaccgagtcggtgctcaatggctcagggcaggagctggacaaacattgacgcgtctctacgtgggggcgcg

SEQ ID NO: 411 - RNA, 153 nt, synthetic construct
  source 1..153: mol_type = other RNA
cttgtccagctcctgccctggttttagagctagaaatagcaagttaaaataaggctagtccgttatcaacttgaaaaagtggcaccgagtcggtgcgtcaatggctcagggcaggagctggacaacattgacgcgtctctacgtgggggcgcg

SEQ ID NO: 412 - RNA, 153 nt, synthetic construct
  source 1..153: mol_type = other RNA
cttgtccagctcctgccctggttttagagctagaaatagcaagttaaaataaggctagtccgttatcaacttgaaaaagtggcaccgagtcggtgctcgtcaatggctcagggcaggagctggaacattgacgcgtctctacgtgggggcgcg

SEQ ID NO: 413 - RNA, 153 nt, synthetic construct
  source 1..153: mol_type = other RNA
cttgtccagctcctgccctggttttagagctagaaatagcaagttaaaataaggctagtccgttatcaacttgaaaaagtggcaccgagtcggtgcgctcgtcaatggctcagggcaggagctaacattgacgcgtctctacgtgggggcgcg

SEQ ID NO: 414 - RNA, 153 nt, synthetic construct
  source 1..153: mol_type = other RNA
cttgtccagctcctgccctggttttagagctagaaatagcaagttaaaataaggctagtccgttatcaacttgaaaaagtggcaccgagtcggtgctgctcgtcaatggctcagggcaggagcaacattgacgcgtctctacgtgggggcgcg

SEQ ID NO: 415 - RNA, 153 nt, synthetic construct
  source 1..153: mol_type = other RNA
cttgtccagctcctgccctggttttagagctagaaatagcaagttaaaataaggctagtccgttatcaacttgaaaaagtggcaccgagtcggtgctctcgtcaatggctcagggcaggagctaacattgacgcgtctctacgtgggggcgcg

SEQ ID NO: 416 - RNA, 153 nt, synthetic construct
  source 1..153: mol_type = other RNA
cttgtccagctcctgccctggttttagagctagaaatagcaagttaaaataaggctagtccgttatcaacttgaaaaagtggcaccgagtcggtgcttcaatggctcagggcaggagctggacaacattgacgcgtctctacgtgggggcgcg

SEQ ID NO: 417 - RNA, 153 nt, synthetic construct
  source 1..153: mol_type = other RNA
cttgtccagctcctgccctggttttagagctagaaatagcaagttaaaataaggctagtccgttatcaacttgaaaaagtggcaccgagtcggtgctcaatgcgtcagggcaggagctggacaaacattgacgcgtctctacgtgggggcgcg

SEQ ID NO: 418 - RNA, 153 nt, synthetic construct
  source 1..153: mol_type = other RNA
cttgtccagctcctgccctggttttagagctagaaatagcaagttaaaataaggctagtccgttatcaacttgaaaaagtggcaccgagtcggtgcgtcaatgcgtcagggcaggagctggacaacattgacgcgtctctacgtgggggcgcg

SEQ ID NO: 419 - RNA, 153 nt, synthetic construct
  source 1..153: mol_type = other RNA
cttgtccagctcctgccctggttttagagctagaaatagcaagttaaaataaggctagtccgttatcaacttgaaaaagtggcaccgagtcggtgcgctcgtcaatgcgtcagggcaggagctaacattgacgcgtctctacgtgggggcgcg

SEQ ID NO: 420 - RNA, 153 nt, synthetic construct
  source 1..153: mol_type = other RNA
cttgtccagctcctgccctggttttagagctagaaatagcaagttaaaataaggctagtccgttatcaacttgaaaaagtggcaccgagtcggtgctgctcgtcaatgcgtcagggcaggagcaacattgacgcgtctctacgtgggggcgcg

SEQ ID NO: 421 - RNA, 154 nt, synthetic construct
  source 1..154: mol_type = other RNA
cttgtccagctcctgccctggttttagagctagaaatagcaagttaaaataaggctagtccgttatcaacttgaaaaagtggcaccgagtcggtgctgctcgtcaatgcctcagggcaggagctaacattgacgcgtctctacgtgggggcgcg

SEQ ID NO: 422 - RNA, 154 nt, synthetic construct
  source 1..154: mol_type = other RNA
cttgtccagctcctgccctggttttagagctagaaatagcaagttaaaataaggctagtccgttatcaacttgaaaaagtggcaccgagtcggtgcgtcaatgcctcagggcaggagctggacaaacattgacgcgtctctacgtgggggcgcg

SEQ ID NO: 423 - RNA, 154 nt, synthetic construct
  source 1..154: mol_type = other RNA
cttgtccagctcctgccctggttttagagctagaaatagcaagttaaaataaggctagtccgttatcaacttgaaaaagtggcaccgagtcggtgctcgtcaatgcctcagggcaggagctggaaacattgacgcgtctctacgtgggggcgcg

SEQ ID NO: 424 - RNA, 154 nt, synthetic construct
  source 1..154: mol_type = other RNA
cttgtccagctcctgccctggttttagagctagaaatagcaagttaaaataaggctagtccgttatcaacttgaaaaagtggcaccgagtcggtgcgctcgtcaatgcctcagggcaggagctgaacattgacgcgtctctacgtgggggcgcg

SEQ ID NO: 425 - RNA, 154 nt, synthetic construct
  source 1..154: mol_type = other RNA
cttgtccagctcctgccctggttttagagctagaaatagcaagttaaaataaggctagtccgttatcaacttgaaaaagtggcaccgagtcggtgctctgctcgtcaatgcctcagggcaggagaacattgacgcgtctctacgtgggggcgcg

SEQ ID NO: 426 - RNA, 154 nt, synthetic construct
  source 1..154: mol_type = other RNA
cttgtccagctcctgccctggttttagagctagaaatagcaagttaaaataaggctagtccgttatcaacttgaaaaagtggcaccgagtcggtgcgtcaatggctcagggcaggagctggacaaacattgacgcgtctctacgtgggggcgcg

SEQ ID NO: 427 - RNA, 154 nt, synthetic construct
  source 1..154: mol_type = other RNA
cttgtccagctcctgccctggttttagagctagaaatagcaagttaaaataaggctagtccgttatcaacttgaaaaagtggcaccgagtcggtgctcgtcaatggctcagggcaggagctggaaacattgacgcgtctctacgtgggggcgcg

SEQ ID NO: 428 - RNA, 154 nt, synthetic construct
  source 1..154: mol_type = other RNA
cttgtccagctcctgccctggttttagagctagaaatagcaagttaaaataaggctagtccgttatcaacttgaaaaagtggcaccgagtcggtgcgctcgtcaatggctcagggcaggagctgaacattgacgcgtctctacgtgggggcgcg

SEQ ID NO: 429 - RNA, 154 nt, synthetic construct
  source 1..154: mol_type = other RNA
cttgtccagctcctgccctggttttagagctagaaatagcaagttaaaataaggctagtccgttatcaacttgaaaaagtggcaccgagtcggtgctgctcgtcaatggctcagggcaggagctaacattgacgcgtctctacgtgggggcgcg

SEQ ID NO: 430 - RNA, 154 nt, synthetic construct
  source 1..154: mol_type = other RNA
cttgtccagctcctgccctggttttagagctagaaatagcaagttaaaataaggctagtccgttatcaacttgaaaaagtggcaccgagtcggtgctctcgtcaatggctcagggcaggagctgaacattgacgcgtctctacgtgggggcgcg

SEQ ID NO: 431 - RNA, 154 nt, synthetic construct
  source 1..154: mol_type = other RNA
cttgtccagctcctgccctggttttagagctagaaatagcaagttaaaataaggctagtccgttatcaacttgaaaaagtggcaccgagtcggtgcttcaatggctcagggcaggagctggacaaacattgacgcgtctctacgtgggggcgcg

SEQ ID NO: 432 - RNA, 154 nt, synthetic construct
  source 1..154: mol_type = other RNA
cttgtccagctcctgccctggttttagagctagaaatagcaagttaaaataaggctagtccgttatcaacttgaaaaagtggcaccgagtcggtgcgtcaatgcgtcagggcaggagctggacaaacattgacgcgtctctacgtgggggcgcg

SEQ ID NO: 433 - RNA, 154 nt, synthetic construct
  source 1..154: mol_type = other RNA
cttgtccagctcctgccctggttttagagctagaaatagcaagttaaaataaggctagtccgttatcaacttgaaaaagtggcaccgagtcggtgctcgtcaatgcgtcagggcaggagctggaaacattgacgcgtctctacgtgggggcgcg

SEQ ID NO: 434 - RNA, 154 nt, synthetic construct
  source 1..154: mol_type = other RNA
cttgtccagctcctgccctggttttagagctagaaatagcaagttaaaataaggctagtccgttatcaacttgaaaaagtggcaccgagtcggtgcgctcgtcaatgcgtcagggcaggagctgaacattgacgcgtctctacgtgggggcgcg

SEQ ID NO: 435 - RNA, 154 nt, synthetic construct
  source 1..154: mol_type = other RNA
cttgtccagctcctgccctggttttagagctagaaatagcaagttaaaataaggctagtccgttatcaacttgaaaaagtggcaccgagtcggtgctgctcgtcaatgcgtcagggcaggagctaacattgacgcgtctctacgtgggggcgcg

SEQ ID NO: 436 - RNA, 155 nt, synthetic construct
  source 1..155: mol_type = other RNA
cttgtccagctcctgccctggttttagagctagaaatagcaagttaaaataaggctagtccgttatcaacttgaaaaagtggcaccgagtcggtgctcgtcaatgcctcagggcaggagctggacaacattgacgcgtctctacgtgggggcgcg

SEQ ID NO: 437 - RNA, 155 nt, synthetic construct
  source 1..155: mol_type = other RNA
cttgtccagctcctgccctggttttagagctagaaatagcaagttaaaataaggctagtccgttatcaacttgaaaaagtggcaccgagtcggtgcgctcgtcaatgcctcagggcaggagctggaacattgacgcgtctctacgtgggggcgcg

SEQ ID NO: 438 - RNA, 155 nt, synthetic construct
  source 1..155: mol_type = other RNA
cttgtccagctcctgccctggttttagagctagaaatagcaagttaaaataaggctagtccgttatcaacttgaaaaagtggcaccgagtcggtgctgctcgtcaatgcctcagggcaggagctgaacattgacgcgtctctacgtgggggcgcg

SEQ ID NO: 439 - RNA, 155 nt, synthetic construct
  source 1..155: mol_type = other RNA
cttgtccagctcctgccctggttttagagctagaaatagcaagttaaaataaggctagtccgttatcaacttgaaaaagtggcaccgagtcggtgctctgctcgtcaatgcctcagggcaggagcaacattgacgcgtctctacgtgggggcgcg

SEQ ID NO: 440 - RNA, 155 nt, synthetic construct
  source 1..155: mol_type = other RNA
cttgtccagctcctgccctggttttagagctagaaatagcaagttaaaataaggctagtccgttatcaacttgaaaaagtggcaccgagtcggtgcgtcaatgcctcagggcaggagctggacaaaacattgacgcgtctctacgtgggggcgcg

SEQ ID NO: 441 - RNA, 155 nt, synthetic construct
  source 1..155: mol_type = other RNA
cttgtccagctcctgccctggttttagagctagaaatagcaagttaaaataaggctagtccgttatcaacttgaaaaagtggcaccgagtcggtgctcgtcaatggctcagggcaggagctggacaacattgacgcgtctctacgtgggggcgcg

SEQ ID NO: 442 - RNA, 155 nt, synthetic construct
  source 1..155: mol_type = other RNA
cttgtccagctcctgccctggttttagagctagaaatagcaagttaaaataaggctagtccgttatcaacttgaaaaagtggcaccgagtcggtgcgctcgtcaatggctcagggcaggagctggaacattgacgcgtctctacgtgggggcgcg

SEQ ID NO: 443 - RNA, 155 nt, synthetic construct
  source 1..155: mol_type = other RNA
cttgtccagctcctgccctggttttagagctagaaatagcaagttaaaataaggctagtccgttatcaacttgaaaaagtggcaccgagtcggtgctgctcgtcaatggctcagggcaggagctgaacattgacgcgtctctacgtgggggcgcg

SEQ ID NO: 444 - RNA, 155 nt, synthetic construct
  source 1..155: mol_type = other RNA
cttgtccagctcctgccctggttttagagctagaaatagcaagttaaaataaggctagtccgttatcaacttgaaaaagtggcaccgagtcggtgctctcgtcaatggctcagggcaggagctggaacattgacgcgtctctacgtgggggcgcg

SEQ ID NO: 445 - RNA, 155 nt, synthetic construct
  source 1..155: mol_type = other RNA
cttgtccagctcctgccctggttttagagctagaaatagcaagttaaaataaggctagtccgttatcaacttgaaaaagtggcaccgagtcggtgctcgtcaatgcgtcagggcaggagctggacaacattgacgcgtctctacgtgggggcgcg

SEQ ID NO: 446 - RNA, 155 nt, synthetic construct
  source 1..155: mol_type = other RNA
cttgtccagctcctgccctggttttagagctagaaatagcaagttaaaataaggctagtccgttatcaacttgaaaaagtggcaccgagtcggtgcgctcgtcaatgcgtcagggcaggagctggaacattgacgcgtctctacgtgggggcgcg

SEQ ID NO: 447 - RNA, 155 nt, synthetic construct
  source 1..155: mol_type = other RNA
cttgtccagctcctgccctggttttagagctagaaatagcaagttaaaataaggctagtccgttatcaacttgaaaaagtggcaccgagtcggtgctgctcgtcaatgcgtcagggcaggagctgaacattgacgcgtctctacgtgggggcgcg

SEQ ID NO: 448 - RNA, 156 nt, synthetic construct
  source 1..156: mol_type = other RNA
cttgtccagctcctgccctggttttagagctagaaatagcaagttaaaataaggctagtccgttatcaacttgaaaaagtggcaccgagtcggtgcgctctgctcgtcaatgcctcagggcaggagaacattgacgcgtctctacgtgggggcgcg

SEQ ID NO: 449 - RNA, 156 nt, synthetic construct
  source 1..156: mol_type = other RNA
cttgtccagctcctgccctggttttagagctagaaatagcaagttaaaataaggctagtccgttatcaacttgaaaaagtggcaccgagtcggtgctgctcgtcaatgcctcagggcaggagctggaacattgacgcgtctctacgtgggggcgcg

SEQ ID NO: 450 - RNA, 156 nt, synthetic construct
  source 1..156: mol_type = other RNA
cttgtccagctcctgccctggttttagagctagaaatagcaagttaaaataaggctagtccgttatcaacttgaaaaagtggcaccgagtcggtgctcgtcaatgcctcagggcaggagctggacaaacattgacgcgtctctacgtgggggcgcg

SEQ ID NO: 451 - RNA, 156 nt, synthetic construct
  source 1..156: mol_type = other RNA
cttgtccagctcctgccctggttttagagctagaaatagcaagttaaaataaggctagtccgttatcaacttgaaaaagtggcaccgagtcggtgcgctcgtcaatgcctcagggcaggagctggaaacattgacgcgtctctacgtgggggcgcg

SEQ ID NO: 452 - RNA, 156 nt, synthetic construct
  source 1..156: mol_type = other RNA
cttgtccagctcctgccctggttttagagctagaaatagcaagttaaaataaggctagtccgttatcaacttgaaaaagtggcaccgagtcggtgctctgctcgtcaatgcctcagggcaggagctaacattgacgcgtctctacgtgggggcgcg

SEQ ID NO: 453 - RNA, 156 nt, synthetic construct
  source 1..156: mol_type = other RNA
cttgtccagctcctgccctggttttagagctagaaatagcaagttaaaataaggctagtccgttatcaacttgaaaaagtggcaccgagtcggtgctcgtcaatggctcagggcaggagctggacaaacattgacgcgtctctacgtgggggcgcg

SEQ ID NO: 454 - RNA, 156 nt, synthetic construct
  source 1..156: mol_type = other RNA
cttgtccagctcctgccctggttttagagctagaaatagcaagttaaaataaggctagtccgttatcaacttgaaaaagtggcaccgagtcggtgcgctcgtcaatggctcagggcaggagctggaaacattgacgcgtctctacgtgggggcgcg

SEQ ID NO: 455 - RNA, 156 nt, synthetic construct
  source 1..156: mol_type = other RNA
cttgtccagctcctgccctggttttagagctagaaatagcaagttaaaataaggctagtccgttatcaacttgaaaaagtggcaccgagtcggtgctgctcgtcaatggctcagggcaggagctggaacattgacgcgtctctacgtgggggcgcg

SEQ ID NO: 456 - RNA, 156 nt, synthetic construct
  source 1..156: mol_type = other RNA
cttgtccagctcctgccctggttttagagctagaaatagcaagttaaaataaggctagtccgttatcaacttgaaaaagtggcaccgagtcggtgctctcgtcaatggctcagggcaggagctggaaacattgacgcgtctctacgtgggggcgcg

SEQ ID NO: 457 - RNA, 156 nt, synthetic construct
  source 1..156: mol_type = other RNA
cttgtccagctcctgccctggttttagagctagaaatagcaagttaaaataaggctagtccgttatcaacttgaaaaagtggcaccgagtcggtgctcgtcaatgcgtcagggcaggagctggacaaacattgacgcgtctctacgtgggggcgcg

SEQ ID NO: 458 - RNA, 156 nt, synthetic construct
  source 1..156: mol_type = other RNA
cttgtccagctcctgccctggttttagagctagaaatagcaagttaaaataaggctagtccgttatcaacttgaaaaagtggcaccgagtcggtgcgctcgtcaatgcgtcagggcaggagctggaaacattgacgcgtctctacgtgggggcgcg

SEQ ID NO: 459 - RNA, 156 nt, synthetic construct
  source 1..156: mol_type = other RNA
cttgtccagctcctgccctggttttagagctagaaatagcaagttaaaataaggctagtccgttatcaacttgaaaaagtggcaccgagtcggtgctgctcgtcaatgcgtcagggcaggagctggaacattgacgcgtctctacgtgggggcgcg

SEQ ID NO: 460 - RNA, 157 nt, synthetic construct
  source 1..157: mol_type = other RNA
cttgtccagctcctgccctggttttagagctagaaatagcaagttaaaataaggctagtccgttatcaacttgaaaaagtggcaccgagtcggtgcgctcgtcaatgcctcagggcaggagctggacaacattgacgcgtctctacgtgggggcgcg

SEQ ID NO: 461 - RNA, 157 nt, synthetic construct
  source 1..157: mol_type = other RNA
cttgtccagctcctgccctggttttagagctagaaatagcaagttaaaataaggctagtccgttatcaacttgaaaaagtggcaccgagtcggtgctgctcgtcaatgcctcagggcaggagctggaaacattgacgcgtctctacgtgggggcgcg

SEQ ID NO: 462 - RNA, 157 nt, synthetic construct
  source 1..157: mol_type = other RNA
cttgtccagctcctgccctggttttagagctagaaatagcaagttaaaataaggctagtccgttatcaacttgaaaaagtggcaccgagtcggtgctctgctcgtcaatgcctcagggcaggagctgaacattgacgcgtctctacgtgggggcgcg

SEQ ID NO: 463 - RNA, 157 nt, synthetic construct
  source 1..157: mol_type = other RNA
cttgtccagctcctgccctggttttagagctagaaatagcaagttaaaataaggctagtccgttatcaacttgaaaaagtggcaccgagtcggtgcgctctgctcgtcaatgcctcagggcaggagcaacattgacgcgtctctacgtgggggcgcg

SEQ ID NO: 464 - RNA, 157 nt, synthetic construct
  source 1..157: mol_type = other RNA
cttgtccagctcctgccctggttttagagctagaaatagcaagttaaaataaggctagtccgttatcaacttgaaaaagtggcaccgagtcggtgctcgtcaatgcctcagggcaggagctggacaaaacattgacgcgtctctacgtgggggcgcg

SEQ ID NO: 465 - RNA, 157 nt, synthetic construct
  source 1..157: mol_type = other RNA
cttgtccagctcctgccctggttttagagctagaaatagcaagttaaaataaggctagtccgttatcaacttgaaaaagtggcaccgagtcggtgcgctcgtcaatggctcagggcaggagctggacaacattgacgcgtctctacgtgggggcgcg

SEQ ID NO: 466 - RNA, 157 nt, synthetic construct
  source 1..157: mol_type = other RNA
cttgtccagctcctgccctggttttagagctagaaatagcaagttaaaataaggctagtccgttatcaacttgaaaaagtggcaccgagtcggtgctgctcgtcaatggctcagggcaggagctggaaacattgacgcgtctctacgtgggggcgcg

SEQ ID NO: 467 - RNA, 157 nt, synthetic construct
  source 1..157: mol_type = other RNA
cttgtccagctcctgccctggttttagagctagaaatagcaagttaaaataaggctagtccgttatcaacttgaaaaagtggcaccgagtcggtgctctcgtcaatggctcagggcaggagctggacaacattgacgcgtctctacgtgggggcgcg

SEQ ID NO: 468 - RNA, 157 nt, synthetic construct
  source 1..157: mol_type = other RNA
cttgtccagctcctgccctggttttagagctagaaatagcaagttaaaataaggctagtccgttatcaacttgaaaaagtggcaccgagtcggtgcgctcgtcaatgcgtcagggcaggagctggacaacattgacgcgtctctacgtgggggcgcg

SEQ ID NO: 469 - RNA, 157 nt, synthetic construct
  source 1..157: mol_type = other RNA
cttgtccagctcctgccctggttttagagctagaaatagcaagttaaaataaggctagtccgttatcaacttgaaaaagtggcaccgagtcggtgctgctcgtcaatgcgtcagggcaggagctggaaacattgacgcgtctctacgtgggggcgcg

SEQ ID NO: 470 - RNA, 158 nt, synthetic construct
  source 1..158: mol_type = other RNA
cttgtccagctcctgccctggttttagagctagaaatagcaagttaaaataaggctagtccgttatcaacttgaaaaagtggcaccgagtcggtgcgagctctgctcgtcaatgcctcagggcaggagaacattgacgcgtctctacgtgggggcgcg

SEQ ID NO: 471 - RNA, 158 nt, synthetic construct
  source 1..158: mol_type = other RNA
cttgtccagctcctgccctggttttagagctagaaatagcaagttaaaataaggctagtccgttatcaacttgaaaaagtggcaccgagtcggtgcgctctgctcgtcaatgcctcagggcaggagctaacattgacgcgtctctacgtgggggcgcg

SEQ ID NO: 472 - RNA, 158 nt, synthetic construct
  source 1..158: mol_type = other RNA
cttgtccagctcctgccctggttttagagctagaaatagcaagttaaaataaggctagtccgttatcaacttgaaaaagtggcaccgagtcggtgcgctcgtcaatgcctcagggcaggagctggacaaacattgacgcgtctctacgtgggggcgcg

SEQ ID NO: 473 - RNA, 158 nt, synthetic construct
  source 1..158: mol_type = other RNA
cttgtccagctcctgccctggttttagagctagaaatagcaagttaaaataaggctagtccgttatcaacttgaaaaagtggcaccgagtcggtgctgctcgtcaatgcctcagggcaggagctggacaacattgacgcgtctctacgtgggggcgcg

SEQ ID NO: 474 - RNA, 158 nt, synthetic construct
  source 1..158: mol_type = other RNA
cttgtccagctcctgccctggttttagagctagaaatagcaagttaaaataaggctagtccgttatcaacttgaaaaagtggcaccgagtcggtgctctgctcgtcaatgcctcagggcaggagctggaacattgacgcgtctctacgtgggggcgcg

SEQ ID NO: 475 - RNA, 158 nt, synthetic construct
  source 1..158: mol_type = other RNA
cttgtccagctcctgccctggttttagagctagaaatagcaagttaaaataaggctagtccgttatcaacttgaaaaagtggcaccgagtcggtgcgctcgtcaatggctcagggcaggagctggacaaacattgacgcgtctctacgtgggggcgcg

SEQ ID NO: 476 - RNA, 158 nt, synthetic construct
  source 1..158: mol_type = other RNA
cttgtccagctcctgccctggttttagagctagaaatagcaagttaaaataaggctagtccgttatcaacttgaaaaagtggcaccgagtcggtgctgctcgtcaatggctcagggcaggagctggacaacattgacgcgtctctacgtgggggcgcg

SEQ ID NO: 477 - RNA, 158 nt, synthetic construct
  source 1..158: mol_type = other RNA
cttgtccagctcctgccctggttttagagctagaaatagcaagttaaaataaggctagtccgttatcaacttgaaaaagtggcaccgagtcggtgctctcgtcaatggctcagggcaggagctggacaaacattgacgcgtctctacgtgggggcgcg

SEQ ID NO: 478 - RNA, 158 nt, synthetic construct
  source 1..158: mol_type = other RNA
cttgtccagctcctgccctggttttagagctagaaatagcaagttaaaataaggctagtccgttatcaacttgaaaaagtggcaccgagtcggtgcgctcgtcaatgcgtcagggcaggagctggacaaacattgacgcgtctctacgtgggggcgcg

SEQ ID NO: 479 - RNA, 158 nt, synthetic construct
  source 1..158: mol_type = other RNA
cttgtccagctcctgccctggttttagagctagaaatagcaagttaaaataaggctagtccgttatcaacttgaaaaagtggcaccgagtcggtgctgctcgtcaatgcgtcagggcaggagctggacaacattgacgcgtctctacgtgggggcgcg

SEQ ID NO: 480 - RNA, 159 nt, synthetic construct
  source 1..159: mol_type = other RNA
cttgtccagctcctgccctggttttagagctagaaatagcaagttaaaataaggctagtccgttatcaacttgaaaaagtggcaccgagtcggtgcgctcgtcaatgcctcagggcaggagctggacaaaacattgacgcgtctctacgtgggggcgcg

SEQ ID NO: 481 - RNA, 159 nt, synthetic construct
  source 1..159: mol_type = other RNA
cttgtccagctcctgccctggttttagagctagaaatagcaagttaaaataaggctagtccgttatcaacttgaaaaagtggcaccgagtcggtgctgctcgtcaatgcctcagggcaggagctggacaaacattgacgcgtctctacgtgggggcgcg

SEQ ID NO: 482 - RNA, 159 nt, synthetic construct
  source 1..159: mol_type = other RNA
cttgtccagctcctgccctggttttagagctagaaatagcaagttaaaataaggctagtccgttatcaacttgaaaaagtggcaccgagtcggtgctctgctcgtcaatgcctcagggcaggagctggaaacattgacgcgtctctacgtgggggcgcg

SEQ ID NO: 483 - RNA, 159 nt, synthetic construct
  source 1..159: mol_type = other RNA
cttgtccagctcctgccctggttttagagctagaaatagcaagttaaaataaggctagtccgttatcaacttgaaaaagtggcaccgagtcggtgcgctctgctcgtcaatgcctcagggcaggagctgaacattgacgcgtctctacgtgggggcgcg

SEQ ID NO: 484 - RNA, 159 nt, synthetic construct
  source 1..159: mol_type = other RNA
cttgtccagctcctgccctggttttagagctagaaatagcaagttaaaataaggctagtccgttatcaacttgaaaaagtggcaccgagtcggtgcgagctctgctcgtcaatgcctcagggcaggagcaacattgacgcgtctctacgtgggggcgcg

SEQ ID NO: 485 - RNA, 159 nt, synthetic construct
  source 1..159: mol_type = other RNA
cttgtccagctcctgccctggttttagagctagaaatagcaagttaaaataaggctagtccgttatcaacttgaaaaagtggcaccgagtcggtgctgctcgtcaatggctcagggcaggagctggacaaacattgacgcgtctctacgtgggggcgcg

SEQ ID NO: 486 - RNA, 159 nt, synthetic construct
  source 1..159: mol_type = other RNA
cttgtccagctcctgccctggttttagagctagaaatagcaagttaaaataaggctagtccgttatcaacttgaaaaagtggcaccgagtcggtgctgctcgtcaatgcgtcagggcaggagctggacaaacattgacgcgtctctacgtgggggcgcg

SEQ ID NO: 487 - RNA, 160 nt, synthetic construct
  source 1..160: mol_type = other RNA
cttgtccagctcctgccctggttttagagctagaaatagcaagttaaaataaggctagtccgttatcaacttgaaaaagtggcaccgagtcggtgcgggagctctgctcgtcaatgcctcagggcaggagaacattgacgcgtctctacgtgggggcgcg

SEQ ID NO: 488 - RNA, 160 nt, synthetic construct
  source 1..160: mol_type = other RNA
cttgtccagctcctgccctggttttagagctagaaatagcaagttaaaataaggctagtccgttatcaacttgaaaaagtggcaccgagtcggtgcgagctctgctcgtcaatgcctcagggcaggagctaacattgacgcgtctctacgtgggggcgcg

SEQ ID NO: 489 - RNA, 160 nt, synthetic construct
  source 1..160: mol_type = other RNA
cttgtccagctcctgccctggttttagagctagaaatagcaagttaaaataaggctagtccgttatcaacttgaaaaagtggcaccgagtcggtgcgctctgctcgtcaatgcctcagggcaggagctggaacattgacgcgtctctacgtgggggcgcg

SEQ ID NO: 490 - RNA, 160 nt, synthetic construct
  source 1..160: mol_type = other RNA
cttgtccagctcctgccctggttttagagctagaaatagcaagttaaaataaggctagtccgttatcaacttgaaaaagtggcaccgagtcggtgctctgctcgtcaatgcctcagggcaggagctggacaacattgacgcgtctctacgtgggggcgcg

SEQ ID NO: 491 - RNA, 160 nt, synthetic construct
  source 1..160: mol_type = other RNA
cttgtccagctcctgccctggttttagagctagaaatagcaagttaaaataaggctagtccgttatcaacttgaaaaagtggcaccgagtcggtgctgctcgtcaatgcctcagggcaggagctggacaaaacattgacgcgtctctacgtgggggcgcg

SEQ ID NO: 492 - RNA, 161 nt, synthetic construct
  source 1..161: mol_type = other RNA
cttgtccagctcctgccctggttttagagctagaaatagcaagttaaaataaggctagtccgttatcaacttgaaaaagtggcaccgagtcggtgctctgctcgtcaatgcctcagggcaggagctggacaaacattgacgcgtctctacgtgggggcgcg

SEQ ID NO: 493 - RNA, 161 nt, synthetic construct
  source 1..161: mol_type = other RNA
cttgtccagctcctgccctggttttagagctagaaatagcaagttaaaataaggctagtccgttatcaacttgaaaaagtggcaccgagtcggtgcgctctgctcgtcaatgcctcagggcaggagctggaaacattgacgcgtctctacgtgggggcgcg

SEQ ID NO: 494 - RNA, 161 nt, synthetic construct
  source 1..161: mol_type = other RNA
cttgtccagctcctgccctggttttagagctagaaatagcaagttaaaataaggctagtccgttatcaacttgaaaaagtggcaccgagtcggtgcgagctctgctcgtcaatgcctcagggcaggagctgaacattgacgcgtctctacgtgggggcgcg

SEQ ID NO: 495 - RNA, 161 nt, synthetic construct
  source 1..161: mol_type = other RNA
cttgtccagctcctgccctggttttagagctagaaatagcaagttaaaataaggctagtccgttatcaacttgaaaaagtggcaccgagtcggtgcgggagctctgctcgtcaatgcctcagggcaggagcaacattgacgcgtctctacgtgggggcgcg

SEQ ID NO: 496 - RNA, 162 nt, synthetic construct
  source 1..162: mol_type = other RNA
cttgtccagctcctgccctggttttagagctagaaatagcaagttaaaataaggctagtccgttatcaacttgaaaaagtggcaccgagtcggtgcgggagctctgctcgtcaatgcctcagggcaggagctaacattgacgcgtctctacgtgggggcgcg

SEQ ID NO: 497 - RNA, 162 nt, synthetic construct
  source 1..162: mol_type = other RNA
cttgtccagctcctgccctggttttagagctagaaatagcaagttaaaataaggctagtccgttatcaacttgaaaaagtggcaccgagtcggtgcgagctctgctcgtcaatgcctcagggcaggagctggaacattgacgcgtctctacgtgggggcgcg

SEQ ID NO: 498 - RNA, 162 nt, synthetic construct
  source 1..162: mol_type = other RNA
cttgtccagctcctgccctggttttagagctagaaatagcaagttaaaataaggctagtccgttatcaacttgaaaaagtggcaccgagtcggtgcgctctgctcgtcaatgcctcagggcaggagctggacaacattgacgcgtctctacgtgggggcgcg

SEQ ID NO: 499 - RNA, 162 nt, synthetic construct
  source 1..162: mol_type = other RNA
cttgtccagctcctgccctggttttagagctagaaatagcaagttaaaataaggctagtccgttatcaacttgaaaaagtggcaccgagtcggtgctctgctcgtcaatgcctcagggcaggagctggacaaaacattgacgcgtctctacgtgggggcgcg

SEQ ID NO: 500 - RNA, 163 nt, synthetic construct
  source 1..163: mol_type = other RNA
cttgtccagctcctgccctggttttagagctagaaatagcaagttaaaataaggctagtccgttatcaacttgaaaaagtggcaccgagtcggtgcgctctgctcgtcaatgcctcagggcaggagctggacaaacattgacgcgtctctacgtgggggcgcg

SEQ ID NO: 501 - RNA, 163 nt, synthetic construct
  source 1..163: mol_type = other RNA
cttgtccagctcctgccctggttttagagctagaaatagcaagttaaaataaggctagtccgttatcaacttgaaaaagtggcaccgagtcggtgcgagctctgctcgtcaatgcctcagggcaggagctggaaacattgacgcgtctctacgtgggggcgcg

SEQ ID NO: 502 - RNA, 164 nt, synthetic construct
  source 1..164: mol_type = other RNA
cttgtccagctcctgccctggttttagagctagaaatagcaagttaaaataaggctagtccgttatcaacttgaaaaagtggcaccgagtcggtgcgggagctctgctcgtcaatgcctcagggcaggagctggaacattgacgcgtctctacgtgggggcgcg

SEQ ID NO: 503 - RNA, 164 nt, synthetic construct
  source 1..164: mol_type = other RNA
cttgtccagctcctgccctggttttagagctagaaatagcaagttaaaataaggctagtccgttatcaacttgaaaaagtggcaccgagtcggtgcgagctctgctcgtcaatgcctcagggcaggagctggacaacattgacgcgtctctacgtgggggcgcg

SEQ ID NO: 504 - RNA, 164 nt, synthetic construct
  source 1..164: mol_type = other RNA
cttgtccagctcctgccctggttttagagctagaaatagcaagttaaaataaggctagtccgttatcaacttgaaaaagtggcaccgagtcggtgcgctctgctcgtcaatgcctcagggcaggagctggacaaaacattgacgcgtctctacgtgggggcgcg

SEQ ID NO: 505 - RNA, 165 nt, synthetic construct
  source 1..165: mol_type = other RNA
cttgtccagctcctgccctggttttagagctagaaatagcaagttaaaataaggctagtccgttatcaacttgaaaaagtggcaccgagtcggtgcgagctctgctcgtcaatgcctcagggcaggagctggacaaacattgacgcgtctctacgtgggggcgcg

SEQ ID NO: 506 - RNA, 166 nt, synthetic construct
  source 1..166: mol_type = other RNA
cttgtccagctcctgccctggttttagagctagaaatagcaagttaaaataaggctagtccgttatcaacttgaaaaagtggcaccgagtcggtgcgggagctctgctcgtcaatgcctcagggcaggagctggacaacattgacgcgtctctacgtgggggcgcg

SEQ ID NO: 507 - RNA, 166 nt, synthetic construct
  source 1..166: mol_type = other RNA
cttgtccagctcctgccctggttttagagctagaaatagcaagttaaaataaggctagtccgttatcaacttgaaaaagtggcaccgagtcggtgcgagctctgctcgtcaatgcctcagggcaggagctggacaaaacattgacgcgtctctacgtgggggcgcg

SEQ ID NO: 508 - RNA, 168 nt, synthetic construct
  source 1..168: mol_type = other RNA
cttgtccagctcctgccctggttttagagctagaaatagcaagttaaaataaggctagtccgttatcaacttgaaaaagtggcaccgagtcggtgcgggagctctgctcgtcaatgcctcagggcaggagctggacaaaacattgacgcgtctctacgtgggggcgcg

SEQ ID NO: 509 - RNA, 96 nt, synthetic construct
  source 1..96: mol_type = other RNA
acagtggctttgatccacaagttttagagctagaaatagcaagttaaaataaggctagtccgttatcaacttgaaaaagtggcaccgagtcggtgc

SEQ ID NO: 510 - RNA, 96 nt, synthetic construct
  source 1..96: mol_type = other RNA
acatgcaaaactgaacactcgttttagagctagaaatagcaagttaaaataaggctagtccgttatcaacttgaaaaagtggcaccgagtcggtgc

SEQ ID NO: 511 - RNA, 96 nt, synthetic construct
  source 1..96: mol_type = other RNA
actgcctcacaccctctccggttttagagctagaaatagcaagttaaaataaggctagtccgttatcaacttgaaaaagtggcaccgagtcggtgc

SEQ ID NO: 512 - RNA, 96 nt, synthetic construct
  source 1..96: mol_type = other RNA
agccactgtcctctgcaaaagttttagagctagaaatagcaagttaaaataaggctagtccgttatcaacttgaaaaagtggcaccgagtcggtgc

SEQ ID NO: 513 - RNA, 96 nt, synthetic construct
  source 1..96: mol_type = other RNA
atgcagtacgggcttttccagttttagagctagaaatagcaagttaaaataaggctagtccgttatcaacttgaaaaagtggcaccgagtcggtgc

SEQ ID NO: 514 - RNA, 96 nt, synthetic construct
  source 1..96: mol_type = other RNA
caaggcctcagggcaggagcgttttagagctagaaatagcaagttaaaataaggctagtccgttatcaacttgaaaaagtggcaccgagtcggtgc

SEQ ID NO: 515 - RNA, 96 nt, synthetic construct
  source 1..96: mol_type = other RNA
caaggctgtcacaactccgagttttagagctagaaatagcaagttaaaataaggctagtccgttatcaacttgaaaaagtggcaccgagtcggtgc

SEQ ID NO: 516 - RNA, 96 nt, synthetic construct
  source 1..96: mol_type = other RNA
caatgcctcagggcaggagcgttttagagctagaaatagcaagttaaaataaggctagtccgttatcaacttgaaaaagtggcaccgagtcggtgc

SEQ ID NO: 517 - RNA, 96 nt, synthetic construct
  source 1..96: mol_type = other RNA
cagccttggggacaccgttggttttagagctagaaatagcaagttaaaataaggctagtccgttatcaacttgaaaaagtggcaccgagtcggtgc

SEQ ID NO: 518 - RNA, 96 nt, synthetic construct
  source 1..96: mol_type = other RNA
caggagctggacaagtttatgttttagagctagaaatagcaagttaaaataaggctagtccgttatcaacttgaaaaagtggcaccgagtcggtgc

SEQ ID NO: 519 - RNA, 96 nt, synthetic construct
  source 1..96: mol_type = other RNA
ccaaggctgtcacaactccggttttagagctagaaatagcaagttaaaataaggctagtccgttatcaacttgaaaaagtggcaccgagtcggtgc

SEQ ID NO: 520 - RNA, 96 nt, synthetic construct
  source 1..96: mol_type = other RNA
cctcggagttgtgacagcctgttttagagctagaaatagcaagttaaaataaggctagtccgttatcaacttgaaaaagtggcaccgagtcggtgc

SEQ ID NO: 521 - RNA, 96 nt, synthetic construct
  source 1..96: mol_type = other RNA
ctacttacatgagaaccgaagttttagagctagaaatagcaagttaaaataaggctagtccgttatcaacttgaaaaagtggcaccgagtcggtgc

SEQ ID NO: 522 - RNA, 96 nt, synthetic construct
  source 1..96: mol_type = other RNA
ctcggagttgtgacagccttgttttagagctagaaatagcaagttaaaataaggctagtccgttatcaacttgaaaaagtggcaccgagtcggtgc

SEQ ID NO: 523 - RNA, 96 nt, synthetic construct
  source 1..96: mol_type = other RNA
ctctgctcgtcaaggcctcagttttagagctagaaatagcaagttaaaataaggctagtccgttatcaacttgaaaaagtggcaccgagtcggtgc

SEQ ID NO: 524 - RNA, 96 nt, synthetic construct
  source 1..96: mol_type = other RNA
ctctgctcgtcaatgcctcagttttagagctagaaatagcaagttaaaataaggctagtccgttatcaacttgaaaaagtggcaccgagtcggtgc

SEQ ID NO: 525 - RNA, 96 nt, synthetic construct
  source 1..96: mol_type = other RNA
ctctgctcgtcaatggctcagttttagagctagaaatagcaagttaaaataaggctagtccgttatcaacttgaaaaagtggcaccgagtcggtgc

SEQ ID NO: 526 - RNA, 96 nt, synthetic construct
  source 1..96: mol_type = other RNA
gagagggtgtgaggcagtgtgttttagagctagaaatagcaagttaaaataaggctagtccgttatcaacttgaaaaagtggcaccgagtcggtgc

SEQ ID NO: 527 - RNA, 96 nt, synthetic construct
  source 1..96: mol_type = other RNA
gatccacaacggtgtccccagttttagagctagaaatagcaagttaaaataaggctagtccgttatcaacttgaaaaagtggcaccgagtcggtgc

SEQ ID NO: 528 - RNA, 96 nt, synthetic construct
  source 1..96: mol_type = other RNA
gcaggagctggacaagtttagttttagagctagaaatagcaagttaaaataaggctagtccgttatcaacttgaaaaagtggcaccgagtcggtgc

SEQ ID NO: 529 - RNA, 96 nt, synthetic construct
  source 1..96: mol_type = other RNA
gccactgtcctctgcaaaacgttttagagctagaaatagcaagttaaaataaggctagtccgttatcaacttgaaaaagtggcaccgagtcggtgc

SEQ ID NO: 530 - RNA, 96 nt, synthetic construct
  source 1..96: mol_type = other RNA
gctcgtcaaggcctcagggcgttttagagctagaaatagcaagttaaaataaggctagtccgttatcaacttgaaaaagtggcaccgagtcggtgc

SEQ ID NO: 531 - RNA, 96 nt, synthetic construct
  source 1..96: mol_type = other RNA
gctcgtcaatgcctcagggcgttttagagctagaaatagcaagttaaaataaggctagtccgttatcaacttgaaaaagtggcaccgagtcggtgc

SEQ ID NO: 532 - RNA, 96 nt, synthetic construct
  source 1..96: mol_type = other RNA
gctcgtcaatggctcagggcgttttagagctagaaatagcaagttaaaataaggctagtccgttatcaacttgaaaaagtggcaccgagtcggtgc

SEQ ID NO: 533 - RNA, 96 nt, synthetic construct
  source 1..96: mol_type = other RNA
gctctgctcgtcaaggcctcgttttagagctagaaatagcaagttaaaataaggctagtccgttatcaacttgaaaaagtggcaccgagtcggtgc

SEQ ID NO: 534 - RNA, 96 nt, synthetic construct
  source 1..96: mol_type = other RNA
gctctgctcgtcaatgcctcgttttagagctagaaatagcaagttaaaataaggctagtccgttatcaacttgaaaaagtggcaccgagtcggtgc

SEQ ID NO: 535 - RNA, 96 nt, synthetic construct
  source 1..96: mol_type = other RNA
ggagagggtgtgaggcagtggttttagagctagaaatagcaagttaaaataaggctagtccgttatcaacttgaaaaagtggcaccgagtcggtgc

SEQ ID NO: 536 - RNA, 96 nt, synthetic construct
  source 1..96: mol_type = other RNA
ggcagtgtgggttgggagcagttttagagctagaaatagcaagttaaaataaggctagtccgttatcaacttgaaaaagtggcaccgagtcggtgc

SEQ ID NO: 537 - RNA, 96 nt, synthetic construct
  source 1..96: mol_type = other RNA
gggtgtgaggcagtgtgggtgttttagagctagaaatagcaagttaaaataaggctagtccgttatcaacttgaaaaagtggcaccgagtcggtgc

SEQ ID NO: 538 - RNA, 96 nt, synthetic construct
  source 1..96: mol_type = other RNA
ggtgtgaggcagtgtgggttgttttagagctagaaatagcaagttaaaataaggctagtccgttatcaacttgaaaaagtggcaccgagtcggtgc

SEQ ID NO: 539 - RNA, 96 nt, synthetic construct
  source 1..96: mol_type = other RNA
ggttgggagcaaggccctttgttttagagctagaaatagcaagttaaaataaggctagtccgttatcaacttgaaaaagtggcaccgagtcggtgc

SEQ ID NO: 540 - RNA, 96 nt, synthetic construct
  source 1..96: mol_type = other RNA
gtacgggcttttccacggaggttttagagctagaaatagcaagttaaaataaggctagtccgttatcaacttgaaaaagtggcaccgagtcggtgc

SEQ ID NO: 541 - RNA, 96 nt, synthetic construct
  source 1..96: mol_type = other RNA
tacgggcttttccacggagagttttagagctagaaatagcaagttaaaataaggctagtccgttatcaacttgaaaaagtggcaccgagtcggtgc

SEQ ID NO: 542 - RNA, 96 nt, synthetic construct
  source 1..96: mol_type = other RNA
tacttacatgagaaccgaaagttttagagctagaaatagcaagttaaaataaggctagtccgttatcaacttgaaaaagtggcaccgagtcggtgc

SEQ ID NO: 543 - RNA, 96 nt, synthetic construct
  source 1..96: mol_type = other RNA
tcggagttgtgacagccttggttttagagctagaaatagcaagttaaaataaggctagtccgttatcaacttgaaaaagtggcaccgagtcggtgc

SEQ ID NO: 544 - RNA, 96 nt, synthetic construct
  source 1..96: mol_type = other RNA
ttatgggtgaaatgcagtacgttttagagctagaaatagcaagttaaaataaggctagtccgttatcaacttgaaaaagtggcaccgagtcggtgc

SEQ ID NO: 545 - RNA, 96 nt, synthetic construct
  source 1..96: mol_type = other RNA
tttatgggtgaaatgcagtagttttagagctagaaatagcaagttaaaataaggctagtccgttatcaacttgaaaaagtggcaccgagtcggtgc

SEQ ID NO: 546 - RNA, 96 nt, synthetic construct
  source 1..96: mol_type = other RNA
ttttccacggagagggtgtggttttagagctagaaatagcaagttaaaataaggctagtccgttatcaacttgaaaaagtggcaccgagtcggtgc

SEQ ID NO: 547 - RNA, 100 nt, synthetic construct
  source 1..100: mol_type = other RNA
agccactgtcctctgcaaaagttttagagctagaaatagcaagttaaaataaggctagtccgttatcaacttgaaaaagtggcaccgagtcggtgctttt

SEQ ID NO: 548 - RNA, 100 nt, synthetic construct
  source 1..100: mol_type = other RNA
caatgcctcagggcaggagcgttttagagctagaaatagcaagttaaaataaggctagtccgttatcaacttgaaaaagtggcaccgagtcggtgctttt

SEQ ID NO: 549 - RNA, 100 nt, synthetic construct
  source 1..100: mol_type = other RNA
cagccttggggacaccgttggttttagagctagaaatagcaagttaaaataaggctagtccgttatcaacttgaaaaagtggcaccgagtcggtgctttt

SEQ ID NO: 550 - RNA, 100 nt, synthetic construct
  source 1..100: mol_type = other RNA
caggagctggacaagtttatgttttagagctagaaatagcaagttaaaataaggctagtccgttatcaacttgaaaaagtggcaccgagtcggtgctttt

SEQ ID NO: 551 - RNA, 100 nt, synthetic construct
  source 1..100: mol_type = other RNA
cctcggagttgtgacagcctgttttagagctagaaatagcaagttaaaataaggctagtccgttatcaacttgaaaaagtggcaccgagtcggtgctttt

SEQ ID NO: 552 - RNA, 100 nt, synthetic construct
  source 1..100: mol_type = other RNA
ctcggagttgtgacagccttgttttagagctagaaatagcaagttaaaataaggctagtccgttatcaacttgaaaaagtggcaccgagtcggtgctttt

SEQ ID NO: 553 - RNA, 100 nt, synthetic construct
  source 1..100: mol_type = other RNA
ctctgctcgtcaatgcctcagttttagagctagaaatagcaagttaaaataaggctagtccgttatcaacttgaaaaagtggcaccgagtcggtgctttt

SEQ ID NO: 554 - RNA, 100 nt, synthetic construct
  source 1..100: mol_type = other RNA
ctctgctcgtcaatggctcagttttagagctagaaatagcaagttaaaataaggctagtccgttatcaacttgaaaaagtggcaccgagtcggtgctttt

SEQ ID NO: 555 - RNA, 100 nt, synthetic construct
  source 1..100: mol_type = other RNA
gcaggagctggacaagtttagttttagagctagaaatagcaagttaaaataaggctagtccgttatcaacttgaaaaagtggcaccgagtcggtgctttt

SEQ ID NO: 556 - RNA, 100 nt, synthetic construct
  source 1..100: mol_type = other RNA
gccactgtcctctgcaaaacgttttagagctagaaatagcaagttaaaataaggctagtccgttatcaacttgaaaaagtggcaccgagtcggtgctttt

SEQ ID NO: 557 - RNA, 100 nt, synthetic construct
  source 1..100: mol_type = other RNA
gctcgtcaatgcctcagggcgttttagagctagaaatagcaagttaaaataaggctagtccgttatcaacttgaaaaagtggcaccgagtcggtgctttt

SEQ ID NO: 558 - RNA, 100 nt, synthetic construct
  source 1..100: mol_type = other RNA
gctcgtcaatggctcagggcgttttagagctagaaatagcaagttaaaataaggctagtccgttatcaacttgaaaaagtggcaccgagtcggtgctttt

SEQ ID NO: 559 - RNA, 100 nt, synthetic construct
  source 1..100: mol_type = other RNA
gctctgctcgtcaatgcctcgttttagagctagaaatagcaagttaaaataaggctagtccgttatcaacttgaaaaagtggcaccgagtcggtgctttt

SEQ ID NO: 560 - RNA, 100 nt, synthetic construct
  source 1..100: mol_type = other RNA
tcggagttgtgacagccttggttttagagctagaaatagcaagttaaaataaggctagtccgttatcaacttgaaaaagtggcaccgagtcggtgctttt

SEQ ID NO: 561 - RNA, 100 nt, synthetic construct
  source 1..100: mol_type = other RNA
ttatgggtgaaatgcagtacgttttagagctagaaatagcaagttaaaataaggctagtccgttatcaacttgaaaaagtggcaccgagtcggtgctttt

SEQ ID NO: 562 - RNA, 100 nt, synthetic construct
  source 1..100: mol_type = other RNA
tttatgggtgaaatgcagtagttttagagctagaaatagcaagttaaaataaggctagtccgttatcaacttgaaaaagtggcaccgagtcggtgctttt

SEQ ID NO: 563 - RNA, 126 nt, synthetic construct
  source 1..126: mol_type = other RNA
acagtggctttgatccacaagttttagagctagaaatagcaagttaaaataaggctagtccgttatcaacttgaaaaagtggcaccgagtcggtgcaacattgacgcgtctctacgtgggggcgcg

SEQ ID NO: 564 - RNA, 126 nt, synthetic construct
  source 1..126: mol_type = other RNA
acatgcaaaactgaacactcgttttagagctagaaatagcaagttaaaataaggctagtccgttatcaacttgaaaaagtggcaccgagtcggtgcaacattgacgcgtctctacgtgggggcgcg

SEQ ID NO: 565 - RNA, 126 nt, synthetic construct
  source 1..126: mol_type = other RNA
actgcctcacaccctctccggttttagagctagaaatagcaagttaaaataaggctagtccgttatcaacttgaaaaagtggcaccgagtcggtgcaacattgacgcgtctctacgtgggggcgcg

SEQ ID NO: 566 - RNA, 126 nt, synthetic construct
  source 1..126: mol_type = other RNA
atgcagtacgggcttttccagttttagagctagaaatagcaagttaaaataaggctagtccgttatcaacttgaaaaagtggcaccgagtcggtgcaacattgacgcgtctctacgtgggggcgcg

SEQ ID NO: 567 - RNA, 126 nt, synthetic construct
  source 1..126: mol_type = other RNA
caaggcctcagggcaggagcgttttagagctagaaatagcaagttaaaataaggctagtccgttatcaacttgaaaaagtggcaccgagtcggtgcaacattgacgcgtctctacgtgggggcgcg

SEQ ID NO: 568 - RNA, 126 nt, synthetic construct
  source 1..126: mol_type = other RNA
caaggctgtcacaactccgagttttagagctagaaatagcaagttaaaataaggctagtccgttatcaacttgaaaaagtggcaccgagtcggtgcaacattgacgcgtctctacgtgggggcgcg

SEQ ID NO: 569 - RNA, 126 nt, synthetic construct
  source 1..126: mol_type = other RNA
caggagctggacaagtttatgttttagagctagaaatagcaagttaaaataaggctagtccgttatcaacttgaaaaagtggcaccgagtcggtgcaacattgacgcgtctctacgtgggggcgcg

SEQ ID NO: 570 - RNA, 126 nt, synthetic construct
  source 1..126: mol_type = other RNA
ccaaggctgtcacaactccggttttagagctagaaatagcaagttaaaataaggctagtccgttatcaacttgaaaaagtggcaccgagtcggtgcaacattgacgcgtctctacgtgggggcgcg

SEQ ID NO: 571 - RNA, 126 nt, synthetic construct
  source 1..126: mol_type = other RNA
ctacttacatgagaaccgaagttttagagctagaaatagcaagttaaaataaggctagtccgttatcaacttgaaaaagtggcaccgagtcggtgcaacattgacgcgtctctacgtgggggcgcg

SEQ ID NO: 572 - RNA, 126 nt, synthetic construct
  source 1..126: mol_type = other RNA
ctctgctcgtcaaggcctcagttttagagctagaaatagcaagttaaaataaggctagtccgttatcaacttgaaaaagtggcaccgagtcggtgcaacattgacgcgtctctacgtgggggcgcg

SEQ ID NO: 573 - RNA, 126 nt, synthetic construct
  source 1..126: mol_type = other RNA
gagagggtgtgaggcagtgtgttttagagctagaaatagcaagttaaaataaggctagtccgttatcaacttgaaaaagtggcaccgagtcggtgcaacattgacgcgtctctacgtgggggcgcg

SEQ ID NO: 574 - RNA, 126 nt, synthetic construct
  source 1..126: mol_type = other RNA
gatccacaacggtgtccccagttttagagctagaaatagcaagttaaaataaggctagtccgttatcaacttgaaaaagtggcaccgagtcggtgcaacattgacgcgtctctacgtgggggcgcg

SEQ ID NO: 575 - RNA, 126 nt, synthetic construct
  source 1..126: mol_type = other RNA
gcaggagctggacaagtttagttttagagctagaaatagcaagttaaaataaggctagtccgttatcaacttgaaaaagtggcaccgagtcggtgcaacattgacgcgtctctacgtgggggcgcg

SEQ ID NO: 576 - RNA, 126 nt, synthetic construct
  source 1..126: mol_type = other RNA
gctcgtcaaggcctcagggcgttttagagctagaaatagcaagttaaaataaggctagtccgttatcaacttgaaaaagtggcaccgagtcggtgcaacattgacgcgtctctacgtgggggcgcg

SEQ ID NO: 577 - RNA, 126 nt, synthetic construct
  source 1..126: mol_type = other RNA
gctctgctcgtcaaggcctcgttttagagctagaaatagcaagttaaaataaggctagtccgttatcaacttgaaaaagtggcaccgagtcggtgcaacattgacgcgtctctacgtgggggcgcg

SEQ ID NO: 578 - RNA, 126 nt, synthetic construct
  source 1..126: mol_type = other RNA
ggagagggtgtgaggcagtggttttagagctagaaatagcaagttaaaataaggctagtccgttatcaacttgaaaaagtggcaccgagtcggtgcaacattgacgcgtctctacgtgggggcgcg

SEQ ID NO: 579 - RNA, 126 nt, synthetic construct
  source 1..126: mol_type = other RNA
ggcagtgtgggttgggagcagttttagagctagaaatagcaagttaaaataaggctagtccgttatcaacttgaaaaagtggcaccgagtcggtgcaacattgacgcgtctctacgtgggggcgcg

SEQ ID NO: 580 - RNA, 126 nt, synthetic construct
  source 1..126: mol_type = other RNA
gggtgtgaggcagtgtgggtgttttagagctagaaatagcaagttaaaataaggctagtccgttatcaacttgaaaaagtggcaccgagtcggtgcaacattgacgcgtctctacgtgggggcgcg

SEQ ID NO: 581 - RNA, 126 nt, synthetic construct
  source 1..126: mol_type = other RNA
ggtgtgaggcagtgtgggttgttttagagctagaaatagcaagttaaaataaggctagtccgttatcaacttgaaaaagtggcaccgagtcggtgcaacattgacgcgtctctacgtgggggcgcg

SEQ ID NO: 582 - RNA, 126 nt, synthetic construct
  source 1..126: mol_type = other RNA
ggttgggagcaaggccctttgttttagagctagaaatagcaagttaaaataaggctagtccgttatcaacttgaaaaagtggcaccgagtcggtgcaacattgacgcgtctctacgtgggggcgcg

SEQ ID NO: 583 - RNA, 126 nt, synthetic construct
  source 1..126: mol_type = other RNA
gtacgggcttttccacggaggttttagagctagaaatagcaagttaaaataaggctagtccgttatcaacttgaaaaagtggcaccgagtcggtgcaacattgacgcgtctctacgtgggggcgcg

SEQ ID NO: 584 - RNA, 126 nt, synthetic construct
  source 1..126: mol_type = other RNA
tacgggcttttccacggagagttttagagctagaaatagcaagttaaaataaggctagtccgttatcaacttgaaaaagtggcaccgagtcggtgcaacattgacgcgtctctacgtgggggcgcg

SEQ ID NO: 585 - RNA, 126 nt, synthetic construct
  source 1..126: mol_type = other RNA
tacttacatgagaaccgaaagttttagagctagaaatagcaagttaaaataaggctagtccgttatcaacttgaaaaagtggcaccgagtcggtgcaacattgacgcgtctctacgtgggggcgcg

SEQ ID NO: 586 - RNA, 126 nt, synthetic construct
  source 1..126: mol_type = other RNA
ttatgggtgaaatgcagtacgttttagagctagaaatagcaagttaaaataaggctagtccgttatcaacttgaaaaagtggcaccgagtcggtgcaacattgacgcgtctctacgtgggggcgcg

SEQ ID NO: 587 - RNA, 126 nt, synthetic construct
  source 1..126: mol_type = other RNA
tttatgggtgaaatgcagtagttttagagctagaaatagcaagttaaaataaggctagtccgttatcaacttgaaaaagtggcaccgagtcggtgcaacattgacgcgtctctacgtgggggcgcg

SEQ ID NO: 588 - RNA, 126 nt, synthetic construct
  source 1..126: mol_type = other RNA
ttttccacggagagggtgtggttttagagctagaaatagcaagttaaaataaggctagtccgttatcaacttgaaaaagtggcaccgagtcggtgcaacattgacgcgtctctacgtgggggcgcg

SEQ ID NO: 589 - protein, 677 aa, Moloney murine leukemia virus
TLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 590 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 591 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 592 - protein, 1368 aa, Streptococcus pyogenes [sequence omitted]

SEQ ID NO: 593 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 594 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 595 - protein, 1054 aa, Staphylococcus lugdunensis
MNQKFILGLDIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGKGSPYGWEGDPKAWYETLMGHCTYFPDELRSVKYAYSADLFNALNDLNNLVIQRDGLSKLEYHEKYHIIENVFKQKKKPTLKQIANEINVNPEDIKGYRITKSGKPQFTEFKLYHDLKSVLFDQSILENEDVLDQIAEILTIYQDKDSIKSKLTELDILLNEEDKENIAQLTGYTGTHRLSLKCIRLVLEEQWYSSRNQMEIFTHLNIKPKKINLTAANKIPKAMIDEFILSPVVKRTFGQAINLINKIIEKYGVPEDIIIELARENNSKDKQKFINEMQKKNENTRKRINEIIGKYGNQNAKRLVEKIRLHDEQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIESKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 596 - protein, 1054 aa, synthetic construct
MNQKFILGLDIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGKGSPYGWEGDPKAWYETLMGHCTYFPDELRSVKYAYSADLFNALNDLNNLVIQRDGLSKLEYHEKYHIIENVFKQKKKPTLKQIANEINVNPEDIKGYRITKSGKPQFTEFKLYHDLKSVLFDQSILENEDVLDQIAEILTIYQDKDSIKSKLTELDILLNEEDKENIAQLTGYTGTHRLSLKCIRLVLEEQWYSSRNQMEIFTHLNIKPKKINLTAANKIPKAMIDEFILSPVVKRTFGQAINLINKIIEKYGVPEDIIIELARENNSKDKQKFINEMQKKNENTRKRINEIIGKYGNQNAKRLVEKIRLHDEQEGKCLYSLESIPLEDLLNNPNHYEVDHIIPRSVSFDNSYHNKVLVKQSEASKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIESKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 597 - protein, 1053 aa, synthetic construct
NQKFILGLDIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGKGSPYGWEGDPKAWYETLMGHCTYFPDELRSVKYAYSADLFNALNDLNNLVIQRDGLSKLEYHEKYHIIENVFKQKKKPTLKQIANEINVNPEDIKGYRITKSGKPQFTEFKLYHDLKSVLFDQSILENEDVLDQIAEILTIYQDKDSIKSKLTELDILLNEEDKENIAQLTGYTGTHRLSLKCIRLVLEEQWYSSRNQMEIFTHLNIKPKKINLTAANKIPKAMIDEFILSPVVKRTFGQAINLINKIIEKYGVPEDIIIELARENNSKDKQKFINEMQKKNENTRKRINEIIGKYGNQNAKRLVEKIRLHDEQEGKCLYSLESIPLEDLLNNPNHYEVDHIIPRSVSFDNSYHNKVLVKQSEASKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIESKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 598 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 599 - protein, 1053 aa, synthetic construct
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 600 - protein, 1052 aa, synthetic construct
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 601 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKVYRSTKEVLDATLIHQSITGLYETRIDLSQLGGD

SEQ ID NO: 602 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKVYRSTKEVLDATLIHQSITGLYETRIDLSQLGGD

SEQ ID NO: 603 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKVYRSTKEVLDATLIHQSITGLYETRIDLSQLGGD

SEQ ID NO: 604 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 605 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 606 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 607 - protein, 1368 aa, synthetic construct
MDKKYSIGLDIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGD

SEQ ID NO: 608 - protein, 1368 aa, synthetic construct
MDKKYSIGLDIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGD

SEQ ID NO: 609 - protein, 1367 aa, synthetic construct
DKKYSIGLDIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGD

SEQ ID NO: 610 - protein, 1057 aa, synthetic construct
MNQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 611 - protein, 1057 aa, synthetic construct
MNQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSEASKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 612 - protein, 1056 aa, synthetic construct
NQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSEASKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIETKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 613 - protein, 1057 aa, synthetic construct
MNQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTSYLKAYFSANNMDVKVKTINGSFTNHLRKVWRFDKYRNHGYKHHAEDALIIANADFLFKENKKLQNTNKILEKPTIENNTKKVTVEKEEDYNNVFETPKLVEDIKQYRDYKFSHRVDKKPNRQLINDTLYSTRMKDEHDYIVQTITDIYGKDNTNLKKQFNKNPEKFLMYQNDPKTFEKLSIIMKQYSDEKNPLAKYYEETGEYLTKYSKKNNGPIVKKIKLLGNKVGNHLDVTNKYENSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 614 - protein, 1057 aa, synthetic construct
MNQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSEASKKSNLTPYQYFNSGKSKLSYNQFKQHILNLSKSQDRISKKKKEYLLEERDINKFEVQKEFINRNLVDTRYATRELTSYLKAYFSANNMDVKVKTINGSFTNHLRKVWRFDKYRNHGYKHHAEDALIIANADFLFKENKKLQNTNKILEKPTIENNTKKVTVEKEEDYNNVFETPKLVEDIKQYRDYKFSHRVDKKPNRQLINDTLYSTRMKDEHDYIVQTITDIYGKDNTNLKKQFNKNPEKFLMYQNDPKTFEKLSIIMKQYSDEKNPLAKYYEETGEYLTKYSKKNNGPIVKKIKLLGNKVGNHLDVTNKYENSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 615 - protein, 1056 aa, synthetic construct
NQKFILGLDIGITSVGYGLIDYETKNIIDAGVRLFPEANVENNEGRRSKRGSRRLKRRRIHRLERVKLLLTEYDLINKEQIPTSNNPYQIRVKGLSEILSKDELAIALLHLAKRRGIHNVDVAADKEETASDSLSTKDQINKNAKFLESRYVCELQKERLENEGHVRGVENRFLTKDIVREAKKIIDTQMQYYPEIDETFKEKYISLVETRREYFEGPGQGSPFGWNGDLKKWYEMLMGHCTYFPQELRSVKYAYSADLFNALNDLNNLIIQRDNSEKLEYHEKYHIIENVFKQKKKPTLKQIAKEIGVNPEDIKGYRITKSGTPEFTSFKLFHDLKKVVKDHAILDDIDLLNQIAEILTIYQDKDSIVAELGQLEYLMSEADKQSISELTGYTGTHSLSLKCMNMIIDELWHSSMNQMEVFTYLNMRPKKYELKGYQRIPTDMIDDAILSPVVKRTFIQSINVINKVIEKYGIPEDIIIELARENNSDDRKKFINNLQKKNEATRKRINEIIGQTGNQNAKRIVEKIRLHDQQEGKCLYSLESIPLEDLLNNPNHYEVDHIIPRSVSFDNSYHNKVLVKQSEASKKSNLTPYQYFNSGKSKLSYNQFKQHILNLSKSQDRISKKKKEYLLEERDINKFEVQKEFINRNLVDTRYATRELTSYLKAYFSANNMDVKVKTINGSFTNHLRKVWRFDKYRNHGYKHHAEDALIIANADFLFKENKKLQNTNKILEKPTIENNTKKVTVEKEEDYNNVFETPKLVEDIKQYRDYKFSHRVDKKPNRQLINDTLYSTRMKDEHDYIVQTITDIYGKDNTNLKKQFNKNPEKFLMYQNDPKTFEKLSIIMKQYSDEKNPLAKYYEETGEYLTKYSKKNNGPIVKKIKLLGNKVGNHLDVTNKYENSTKKLVKLSIKNYRFDVYLTEKGYKFVTIAYLNVFKKDNYYYIPKDKYQELKEKKKIKDTDQFIASFYKNDLIKLNGDLYKIIGVNSDDRNIIELDYYDIKYKDYCEINNIKGEPRIKKTIGKKTESIEKFTTDVLGNLYLHSTEKAPQLIFKRGL

SEQ ID NO: 616 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 617 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 618 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 619 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 620 - protein, 2117 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 621 - protein, 2116 aa, synthetic construct
KRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 622 - protein, 2131 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFESPKKKRKVGSGPAAKRVKLD

SEQ ID NO: 623 - protein, 2130 aa, synthetic construct
KRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFESPKKKRKVGSGPAAKRVKLD

SEQ ID NO: 624 - protein, 7 aa, synthetic construct
PKKKRKV

SEQ ID NO: 625 - protein, 19 aa, synthetic construct
MKRTADGSEFESPKKKRKV

SEQ ID NO: 626 - protein, 30 aa, synthetic construct
MDSLLMNRRKFLYQFKNVRWAKGRRETYLC

SEQ ID NO: 627 - protein, 20 aa, synthetic construct
AVKRPAATKKAGQAKKKKLD

SEQ ID NO: 628 - protein, 25 aa, synthetic construct
MSRRRKANPTKLSENAKKLAKEVEN

SEQ ID NO: 629 - protein, 9 aa, synthetic construct
PAAKRVKLD

SEQ ID NO: 630 - protein, 9 aa, synthetic construct
KLKIKRPVK

SEQ ID NO: 631 - protein, 8 aa, synthetic construct
VSRKRPRP

SEQ ID NO: 632 - protein, 16 aa, synthetic construct
RQARRNRRRRWRERNR

SEQ ID NO: 633 - protein, 10 aa, synthetic construct
EGAPPAKRAR

SEQ ID NO: 634 - protein, 12 aa, synthetic construct
PPQPKKKPLDGE

SEQ ID NO: 635 - protein, 21 aa, synthetic construct
SGGSKRTADGSEFEPKKKRKV

SEQ ID NO: 636 - protein, 17 aa, synthetic construct
KRTADGSEFEPKKKRKV

SEQ ID NO: 637 - protein, 18 aa, synthetic construct
KRTADGSEFESPKKKRKV

SEQ ID NO: 638 - protein, 17 aa, synthetic construct
SKRPAAIKKAGQAKKKK

SEQ ID NO: 639 - protein, 19 aa, synthetic construct
NLSKRPAAIKKAGQAKKKK

SEQ ID NO: 640 - protein, 11 aa, synthetic construct
RQRRNELKRSF

SEQ ID NO: 641 - protein, 38 aa, synthetic construct
NQSSNFGPMKGGNFGGRSSGPYGGGGQYFAKPRNQGGY

SEQ ID NO: 642 - protein, 7 aa, synthetic construct
PKKKRKV

SEQ ID NO: 643 - protein, 16 aa, synthetic construct
KRXXXXXXXXXXKKKL

SEQ ID NO: 644 - protein, 16 aa, synthetic construct
KRPAATKKAGQAKKKK

SEQ ID NO: 645 - RNA, 33 nt, synthetic construct
  source 1..33: mol_type = other RNA
gccaacatgaggatcacccatgtctgcagggcc

SEQ ID NO: 646 - protein, 131 aa, synthetic construct
GSASNFTQFVLVDNGGTGDVTVAPSNFANGVAEWISSNSRSQAYKVTCSVRQSSAQNRKYTIKVEVPKVATQTVGGEELPVAGWRSYLNMELTIPIFATNSDCELIVKAMQGLLKDGNPIPSAIAANSGIY

SEQ ID NO: 647 - protein, 150 aa, synthetic construct
  SITE 1..150: note = This sequence may encompass 1-30 GGGGS repeating units
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 648 - protein, 30 aa, synthetic construct
  SITE 1..30: note = This sequence may encompass 1-30 residues
GGGGGGGGGGGGGGGGGGGGGGGGGGGGGG

SEQ ID NO: 649 - protein, 150 aa, synthetic construct
  SITE 1..150: note = This sequence may encompass 1-30 EAAAK repeating units
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 650 - protein, 90 aa, synthetic construct
  SITE 1..90: note = This sequence may encompass 1-30 GGS repeating units
GGSGGSGGSGGSGGSGGSGGSGGSGGSGGSGGSGGSGGSGGSGGSGGSGGSGGSGGSGGSGGSGGSGGSGGSGGSGGSGGSGGSGGSGGS

SEQ ID NO: 651 - protein, 120 aa, synthetic construct
  SITE 1..120: note = This sequence may encompass 1-30 SGGS repeating units
SGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGS

SEQ ID NO: 652 - protein, 60 aa, synthetic construct
  SITE 1..60: note = This sequence may encompass 1-30 XP repeating units wherein X is any amino acid
XPXPXPXPXPXPXPXPXPXPXPXPXPXPXPXPXPXPXPXPXPXPXPXPXPXPXPXPXPXP

SEQ ID NO: 653 - protein, 21 aa, synthetic construct
  SITE 1..21: note = This sequence may encompass 1, 3 or 7 GGS repeating units
GGSGGSGGSGGSGGSGGSGGS

SEQ ID NO: 654 - protein, 16 aa, synthetic construct
SGSETPGTSESATPES

SEQ ID NO: 655 - protein, 32 aa, synthetic construct
SGGSSGGSSGSETPGTSESATPESSGGSSGGS

SEQ ID NO: 656 - protein, 10 aa, synthetic construct
SGGSGGSGGS

SEQ ID NO: 657 - protein, 4 aa, synthetic construct
SGGS

SEQ ID NO: 658 - protein, 60 aa, synthetic construct
SGGSSGGSSGSETPGTSESATPESAGSYPYDVPDYAGSAAPAAKKKKLDGSGSGGSSGGS

SEQ ID NO: 659 - protein, 6 aa, synthetic construct
GGSGGS

SEQ ID NO: 660 - protein, 9 aa, synthetic construct
GGSGGSGGS

SEQ ID NO: 661 - protein, 33 aa, synthetic construct
SGGSSGGSSGSETPGTSESATPESSGGSSGGSS

SEQ ID NO: 662 - protein, 22 aa, synthetic construct
SGGSKRTADGSEFESPKKKRKV

SEQ ID NO: 663 - protein, 12 aa, synthetic construct
GSGPAAKRVKLD

SEQ ID NO: 664 - protein, 34 aa, synthetic construct
SGGSSGGSKRTADGSEFESPKKKRKVSGGSSGGS

SEQ ID NO: 665 - RNA, 76 nt, synthetic construct
  source 1..76: mol_type = other RNA
gttttagagctagaaatagcaagttaaaataaggctagtccgttatcaacttgaaaaagtggcaccgagtcggtgc

SEQ ID NO: 666 - RNA, 76 nt, synthetic construct
  source 1..76: mol_type = other RNA
gtttgagagctagaaatagcaagtttaaataaggctagtccgttatcaacttgaaaaagtgggaccgagtcggtcc

SEQ ID NO: 667 - RNA, 86 nt, synthetic construct
  source 1..86: mol_type = other RNA
gtttaagagctatgctggaaacagcatagcaagtttaaataaggctagtccgttatcaacttgaaaaagtggcaccgagtcggtgc

SEQ ID NO: 668 - RNA, 76 nt, synthetic construct
  source 1..76: mol_type = other RNA
gttttagtactctggaaacagaatctactaaaacaaggcaaaatgccgtgtttatctcgtcaacttgttggcgaga

SEQ ID NO: 669 - RNA, 76 nt, synthetic construct
  source 1..76: mol_type = other RNA
gttttagtactctggaaacagaatctactgaaacaagacaatatgtcgtgtttatcccatcaatttattggtggga

SEQ ID NO: 670 - RNA, 30 nt, synthetic construct
  source 1..30: mol_type = other RNA
aacattgacgcgtctctacgtgggggcgcg

SEQ ID NO: 671 - skipped

SEQ ID NO: 672 - RNA, 22 nt, synthetic construct
  source 1..22: mol_type = other RNA
cgcgtctctacgtgggggcgcg

SEQ ID NO: 673 - protein, 16 aa, synthetic construct
RQIKIWFQNRRMKWKK

SEQ ID NO: 674 - protein, 232 aa, Homo sapiens
MPSQQKKIIFCMAGVFSFACALGVVTALGTPLWIKATVLCKTGALLVNASGQELDKFMGEMQYGLFHGEGVRQCGLGARPFRFSFFPDLLKAIPVSIHVNVILFSAILIVLTMVGTAFFMYNAFGKPFETLHGPLGLYLLSFISGSCGCLVMILFASEVKIHHLSEKIANYKEGTYVYKTQSEKYTTSFWVIFFCFFVHFLNGLLIRLAGFQFPFAKSKDAETTNVAADLMY

SEQ ID NO: 675 - RNA, 2087 nt, Homo sapiens
  source 1..2087: mol_type = mRNA
acagaagccgtttctcatcatgccaagccaacagaagaaaatcattttttgcatggccggagtgttcagttttgcatgtgccctcggagttgtgacagccttggggacaccgttgtggatcaaagccactgtcctctgcaaaacgggagctctgctcgtcaatgcctcagggcaggagctggacaagtttatgggtgaaatgcagtacgggcttttccacggagagggtgtgaggcagtgtgggttgggagcaaggccctttcggttctcattttttccagatttgctcaaagcaatcccagtgagcatccacgtcaatgtcattctcttctctgccatccttattgtgttaaccatggtggggacagccttcttcatgtacaatgcttttggaaaaccttttgaaactctgcatggtcccctagggctgtaccttttgagcttcatttcaggctcctgtggctgtcttgtcatgatattgtttgcctctgaagtgaaaatccatcacctctcagaaaaaattgcaaattataaagaagggacttatgtctacaaaacgcaaagtgaaaaatataccacctcattctgggtcattttcttttgcttttttgttcattttctgaatgggctcctaatacgacttgctggatttcagttcccttttgcaaaatctaaagacgcagaaacaactaatgtagctgcagatctaatgtactgaaaggcaaacctttctataattttacaagggagtagacttgctttggtcacttttagatgtggttaattttgcatatccttttagtctgcatatattaaagcatcaggacccttcgtgacaatgtttacaaattacgtactaaggatacaggctggaaagtaagggaagcagaaggaaggctttgaaaagttgttttatctggtgggaaattgcttgacccaggtagtcaaaggcagttgactagaatcgacaaattgttactccatatatatatatgtgtgtgtgtgtgtgtgtgtgtgtgtgtaagatgtcttcctatcaaaaagatatcaaaggcacatggaatatattttaataaaaacaaataatatctctaatatatccacacatttgttgccagatttcagaaaactgagctgcaatcgctttcctaaaacagtagtgtattaaatgaacatctataaaatgtatcaacacacattttaaaaaatttgtttaaagtatactcttaggccaggcgtggtgactcacacctgtaattccagcacttcaggaggccaaggtgggaagatcatttgagttcaggagttcgagttacagcctgggcaataaagtgagaccctgtcactaacaaaattaaaaaataaaataaatataaaatataggctttaaaaaagcatagtcttattaaccatgtctgttggtcaaaatctgcaaactctaaaagaagaaaagaagaaaaaaccaagcttagggtatttttcctcccgtgcctgagtcccaattacattcacgacagtactttcaatgaacataattgttaggaccactgaggaatcatgaaaaatgatctctgcttagtacatttgatgcaaaatgacttattaggggctgtttttctagctatagtgtctcgagtactaatatgcaattatgaaaattatattaaatctgggattatgacggtatcactgtatcatcttggtcttgttctggctgtcaccaagcatgacccaggtcaactttttttttcccctgaattacccatcaaattgatctgcagctgactaaaggccacagctgagcctggaactgacccttccttcatcctcaacctgctgtcctccagaaagcaccaaggaaaaagcagagaatgacagcaaacagatcactaggcctctgaccacaggtgctgagtactcagcagccctcatataataggtttgaaagtactccttaaaataaaacactgtttccctttggaactatttacaaggatgaaacaaccgtatacctgagaaataacttgctctggtgtcaattcgctattcgccagcagacatcagaacacaccgagtttccagatgct

SEQ ID NO: 676 - protein, 245 aa, Homo sapiens
MPSQQKKIIFCMAGVFSFACALGVVTALGTPLWIKATVLCKTGALLVNASGQELDKFMGEMQYGLFHGEGVRQCGLGARPFRFSFFPDLLKAIPVSIHVNVILFSAILIVLTMVGTAFFMYNAFGKPFETLHGPLGLYLLSFISVALWLPATRHQAQGSCGCLVMILFASEVKIHHLSEKIANYKEGTYVYKTQSEKYTTSFWVIFFCFFVHFLNGLLIRLAGFQFPFAKSKDAETTNVAADLMY

SEQ ID NO: 677 - RNA, 2398 nt, Homo sapiens
  source 1..2398: mol_type = mRNA
aggagatacttgaaggcagtttgaaagacttgttttacagattcttagtccaaagatttccaattagggagaagaagcagcagaaaaggagaaaagccaagtatgagtgatgatgaggccttcatctactgacatttaacctggcgagaaccgtcgatggtgaagttgccttttcagctgggagctgtccgttcagcttccgtaataaatgcagtcaaagaggcagtcccttcccattgctcacaaaggtcttgtttttgaacctcgccctcacagaagccgtttctcatcatgccaagccaacagaagaaaatcattttttgcatggccggagtgttcagttttgcatgtgccctcggagttgtgacagccttggggacaccgttgtggatcaaagccactgtcctctgcaaaacgggagctctgctcgtcaatgcctcagggcaggagctggacaagtttatgggtgaaatgcagtacgggcttttccacggagagggtgtgaggcagtgtgggttgggagcaaggccctttcggttctcattttttccagatttgctcaaagcaatcccagtgagcatccacgtcaatgtcattctcttctctgccatccttattgtgttaaccatggtggggacagccttcttcatgtacaatgcttttggaaaaccttttgaaactctgcatggtcccctagggctgtaccttttgagcttcatttcagttgccctttggctgccagctaccaggcaccaggctcaaggctcctgtggctgtcttgtcatgatattgtttgcctctgaagtgaaaatccatcacctctcagaaaaaattgcaaattataaagaagggacttatgtctacaaaacgcaaagtgaaaaatataccacctcattctgggtcattttcttttgcttttttgttcattttctgaatgggctcctaatacgacttgctggatttcagttcccttttgcaaaatctaaagacgcagaaacaactaatgtagctgcagatctaatgtactgaaaggcaaacctttctataattttacaagggagtagacttgctttggtcacttttagatgtggttaattttgcatatccttttagtctgcatatattaaagcatcaggacccttcgtgacaatgtttacaaattacgtactaaggatacaggctggaaagtaagggaagcagaaggaaggctttgaaaagttgttttatctggtgggaaattgcttgacccaggtagtcaaaggcagttgactagaatcgacaaattgttactccatatatatatatgtgtgtgtgtgtgtgtgtgtgtgtgtgtaagatgtcttcctatcaaaaagatatcaaaggcacatggaatatattttaataaaaacaaataatatctctaatatatccacacatttgttgccagatttcagaaaactgagctgcaatcgctttcctaaaacagtagtgtattaaatgaacatctataaaatgtatcaacacacattttaaaaaatttgtttaaagtatactcttaggccaggcgtggtgactcacacctgtaattccagcacttcaggaggccaaggtgggaagatcatttgagttcaggagttcgagttacagcctgggcaataaagtgagaccctgtcactaacaaaattaaaaaataaaataaatataaaatataggctttaaaaaagcatagtcttattaaccatgtctgttggtcaaaatctgcaaactctaaaagaagaaaagaagaaaaaaccaagcttagggtatttttcctcccgtgcctgagtcccaattacattcacgacagtactttcaatgaacataattgttaggaccactgaggaatcatgaaaaatgatctctgcttagtacatttgatgcaaaatgacttattaggggctgtttttctagctatagtgtctcgagtactaatatgcaattatgaaaattatattaaatctgggattatgacggtatcactgtatcatcttggtcttgttctggctgtcaccaagcatgacccaggtcaactttttttttcccctgaattacccatcaaattgatctgcagctgactaaaggccacagctgagcctggaactgacccttccttcatcctcaacctgctgtcctccagaaagcaccaaggaaaaagcagagaatgacagcaaacagatcactaggcctctgaccacaggtgctgagtactcagcagccctcatataataggtttgaaagtactccttaaaataaaacactgtttccctttggaactatttacaaggatgaaacaaccgtatacctgagaaataacttgctctggtgtcaattcgctattcgccagcagacatcagaacacaccgagtttccagatgct

SEQ ID NO: 678 - protein, 185 aa, Homo sapiens
MPSQQKKIIFCMAGVFSFACALGVVTALGTPLWIKATVLCKTGALLVNASGQELDKFMGEMQYGLFHGEGVRQCGLGARPFRFSCYFLDPFMGLPTGVPHLLSLPCSTSCRREHTSERVQEPAGCFSAVRSKLHAGPAAATSFSRFAQSNPSEHPRQCHSLLCHPYCVNHGGDSLLHVQCFWKTF

SEQ ID NO: 679 - RNA, 2259 nt, Homo sapiens
  source 1..2259: mol_type = mRNA
acagaagccgtttctcatcatgccaagccaacagaagaaaatcattttttgcatggccggagtgttcagttttgcatgtgccctcggagttgtgacagccttggggacaccgttgtggatcaaagccactgtcctctgcaaaacgggagctctgctcgtcaatgcctcagggcaggagctggacaagtttatgggtgaaatgcagtacgggcttttccacggagagggtgtgaggcagtgtgggttgggagcaaggccctttcggttctcatgctattttcttgaccccttcatgggactcccaacaggggtaccccatttactcagcctgccctgctcaacctcttgcaggagggagcacacgagtgaacgagtgcaggaaccagctggctgctttagtgctgtgaggagtaaactccatgcaggccctgcagcagcaaccagtttttccagatttgctcaaagcaatcccagtgagcatccacgtcaatgtcattctcttctctgccatccttattgtgttaaccatggtggggacagccttcttcatgtacaatgcttttggaaaaccttttgaaactctgcatggtcccctagggctgtaccttttgagcttcatttcaggctcctgtggctgtcttgtcatgatattgtttgcctctgaagtgaaaatccatcacctctcagaaaaaattgcaaattataaagaagggacttatgtctacaaaacgcaaagtgaaaaatataccacctcattctgggtcattttcttttgcttttttgttcattttctgaatgggctcctaatacgacttgctggatttcagttcccttttgcaaaatctaaagacgcagaaacaactaatgtagctgcagatctaatgtactgaaaggcaaacctttctataattttacaagggagtagacttgctttggtcacttttagatgtggttaattttgcatatccttttagtctgcatatattaaagcatcaggacccttcgtgacaatgtttacaaattacgtactaaggatacaggctggaaagtaagggaagcagaaggaaggctttgaaaagttgttttatctggtgggaaattgcttgacccaggtagtcaaaggcagttgactagaatcgacaaattgttactccatatatatatatgtgtgtgtgtgtgtgtgtgtgtgtgtgtaagatgtcttcctatcaaaaagatatcaaaggcacatggaatatattttaataaaaacaaataatatctctaatatatccacacatttgttgccagatttcagaaaactgagctgcaatcgctttcctaaaacagtagtgtattaaatgaacatctataaaatgtatcaacacacattttaaaaaatttgtttaaagtatactcttaggccaggcgtggtgactcacacctgtaattccagcacttcaggaggccaaggtgggaagatcatttgagttcaggagttcgagttacagcctgggcaataaagtgagaccctgtcactaacaaaattaaaaaataaaataaatataaaatataggctttaaaaaagcatagtcttattaaccatgtctgttggtcaaaatctgcaaactctaaaagaagaaaagaagaaaaaaccaagcttagggtatttttcctcccgtgcctgagtcccaattacattcacgacagtactttcaatgaacataattgttaggaccactgaggaatcatgaaaaatgatctctgcttagtacatttgatgcaaaatgacttattaggggctgtttttctagctatagtgtctcgagtactaatatgcaattatgaaaattatattaaatctgggattatgacggtatcactgtatcatcttggtcttgttctggctgtcaccaagcatgacccaggtcaactttttttttcccctgaattacccatcaaattgatctgcagctgactaaaggccacagctgagcctggaactgacccttccttcatcctcaacctgctgtcctccagaaagcaccaaggaaaaagcagagaatgacagcaaacagatcactaggcctctgaccacaggtgctgagtactcagcagccctcatataataggtttgaaagtactccttaaaataaaacactgtttccctttggaactatttacaaggatgaaacaaccgtatacctgagaaataacttgctctggtgtcaattcgctattcgccagcagacatcagaacacaccgagtttccagatgct

SEQ ID NO: 680 - protein, 9 aa, synthetic construct
RRRRRRRRR

SEQ ID NO: 681 - protein, 8 aa, synthetic construct
RRRRRRRR